(19) United States
(12) Patent Application Publication
(10) Pub. No.: US 2026/0356830 A1
(43) Pub. Date: Nov. 12, 2026

(54) COMPOUNDS FOR MODULATING RET PROTEIN

(71) Applicant: Bristol-Myers Squibb Company, Princeton, NJ (US)

(72) Inventors: Jennifer X. Qiao, Cambridge, MA (US); David K. Williams, Delran, NJ (US); Patrice Gill, Levittown, PA (US); T. G. Murali Dhar, Newtown, PA (US); John S. Tokarski, Princeton, NJ (US); Ashvinikumar V. Gavai, Princeton Junction, NJ (US)

(21) Appl. No.: 19/100,519

(22) PCT Filed: Aug. 2, 2023
(86) PCT No.: PCT/US2023/071535

§ 371 Date: Jan. 31, 2025

Related U.S. Application Data
(60) Provisional application No. 63/370,307, filed on Aug. 3, 2022.

(51) Int. Cl. C07D 471/04; A61K 31/4545; A61K 31/496; A61K 31/513; C07D 519/00

(52) U.S. Cl. CPC C07D 471/04; A61K 31/4545; A61K 31/496; A61K 31/513; C07D 519/00

(57) ABSTRACT

The present disclosure relates to protein degradation-inducing compounds for protooncogene tyrosine-protein kinase receptor (RET), which may be either wild type RET or a mutant form of RET useful in the treatment of diseases and disorders mediated by said protein including compounds of Formula (I).

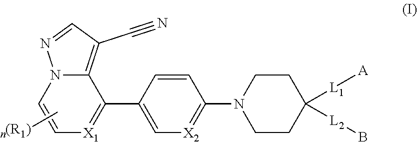

### CROSS-REFERENCE

This application claims the benefit of and priority to U.S. Provisional Patent Application No. 63/370,307, filed on Aug. 3, 2022, the entire contents of which are hereby incorporated by reference herein.

### FIELD OF INVENTION

This disclosure relates to rearranged during transfection (RET) proto-oncogene tyrosine-protein kinase receptor degrading compounds. The degraders described herein are useful in the treatment of diseases and disorders associated with the modulation of RET proteins, which may be either wild type RET or a mutant form of RET. In particular, the invention is concerned with compounds and pharmaceutical compositions that degrade the proto-oncogene tyrosine-protein kinase receptor (RET), which may be either wild type RET or a mutant form of RET, via the ubiquitin proteasome pathway (UPP), methods of treating diseases and disorders associated with said RET protein and pathway using said compounds and pharmaceutical compositions, and methods of synthesizing said compounds and compositions.

### BACKGROUND OF THE INVENTION

Protein degradation is a highly regulated and essential process that maintains cellular homeostasis. The selective identification and removal of damaged, misfolded, or excess proteins is achieved via the ubiquitin-proteasome pathway (UPP). The UPP is central to the regulation of almost all cellular processes, including antigen processing, apoptosis, biogenesis of organelles, cell cycling, DNA transcription and repair, differentiation and development, immune response and inflammation, neural and muscular degeneration, morphogenesis of neural networks, modulation of cell surface receptors, ion channels and the secretory pathway, the response to stress and extracellular modulators, ribosome biogenesis and viral infection. Covalent attachment of multiple ubiquitin molecules by an E3 ubiquitin ligase to a terminal lysine residue marks the protein for proteasome degradation, where the protein is digested into small peptides and eventually into its constituent amino acids that serve as building blocks for new proteins. Defective proteasomal degradation has been linked to a variety of disorders including cancer and others.

Cereblon forms part of an E3 ubiquitin ligase complex which interacts with damaged DNA binding protein 1, forming an E3 ubiquitin ligase complex with Cullin 4 and the E2-binding protein ROC1 (known as RBX1) where it functions as a substrate receptor to select proteins for ubiquitination. The binding of lenalidomide to cereblon facilitates subsequent binding of cereblon to Ikaros and Aiolos, leading to their ubiquitination and degradation by the proteasome (see Lu, G. et al. “The myeloma drug lenalidomide promotes the cereblon-dependent destruction of Ikaros proteins” Science, 2014, 343:305-309; Krönke, J. et al. “Lenalidomide causes selective degradation of IKZF1 and IKZF3 in multiple myeloma cells” Science, 2014, 343:301-305).

The rearranged during transfection (RET) proto-oncogene tyrosine-protein kinase receptor, a cell surface tyrosine kinase receptor, is widely known for its essential role in cell survival, differentiation, proliferation, migration and chemotaxis. RET germline missense and somatic mutations cause medullary thyroid cancer (MTC) and neuroendocrine tumors, whereas RET fusion proteins, overexpression, and copy number gains are present in a broad spectrum of additional cancers such as papillary thyroid cancer, pancreatic cancer, melanoma, leukemia, lung adenocarcinomas, and breast cancer. (Liu Xuan et al., “RET kinase alterations in targeted cancer therapy”, Cancer Drug Resist, 2020; and Mulligan L M., “RET revisited: expanding the oncogenic portfolio”, Nat Rev Cancer., 2014, 14 (3), 173-186).

The academic and clinical interest in RET has led to the identification of several RET mutations that are clinically relevant including RET G810R, RET G810S, and RET G810C. (Solomon, et al., “RET Solvent Front Mutations Mediated Acquired Resistance to Selective RET Inhibition in RET-driven malignancies”, J Thoracic Oncolog., 2020). Treatment of patients with non small cell lung cancer selpercatinib has been shown to cause RET mutations that infer resistances including the RET G810R, RET G810S, and RET G810C mutations.

Other approved tyrosine kinase inhibitors such as sunitinib, sorafenib, ponatinib and lenvatinib have also shown some RET activity in pre-clinical trials and are currently under investigation in numerous phase II clinical trials for treatment of RET fusion positive lung adenocarcinoma (LAD). (Song M., “Progress in Discovery of KIF5B-RET Kinase Inhibitors for the Treatment of Non-Small-Cell Lung Cancer”, J Med Chem., 2015, 58 (9), 3672-3681; Watson A J., et al., “Identification of selective inhibitors of RET and comparison with current clinical candidates through development and validation of a robust screening cascade”, F1000Research 2016, 5:1005).

Despite these efforts, there remains an unmet need in the field new RET modulators to treat disorders mediated by RET in hosts in need thereof, including humans.

### SUMMARY OF THE INVENTION

A first aspect of the instant disclosure is generally directed to compounds of Formula I and pharmaceutically acceptable salts, solvates, isomers, enantiomers, and tautomers described herein that include a Targeting Ligand that binds to RET, an E3 Ligase binding portion (typically via a cereblon subunit), and a Linker that covalently links the Targeting Ligand to the E3 Ligase binding portion.

A second aspect of the instant disclosure is directed to compounds of Formula I:

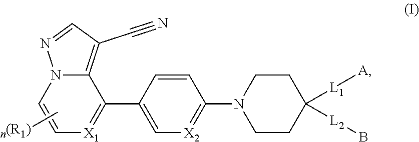

and pharmaceutically acceptable salts, solvates, isomers, enantiomers, and tautomers thereof,
wherein
R₁ is —OCH₂CH₃, —OCH₃, —OCF₃, —OCH₂CH₂OH, —OCH₂CH₂F, 1-methylpyrazol-4-yl, or halogen;
A is independently H, OH, C₁-C₆ alkyl, C₃-C₈ cycloalkyl, aryl, or heteroaryl, wherein the alkyl, cycloalkyl, aryl or heteroaryl is optionally substituted with one or more R₂; wherein R₂ is independently halogen, OH, C₁-C₃ alkyl, or C₁-C₃ alkoxy;
B is a moiety that is capable of binding a E3 ligase;
L₁ is independently a bond, —C₁-C₃ alkylenyl-, —NHCO—, —CONH—, —O—, or —NH—;
L₂ is independently —(CH₂)ₚ-(4- to 12-heterocycloalkyl)-Y₁—C(O)—Y₂—, —(CH₂)ₚ-(4- to 12-heterocycloalkyl)-C(O)—Y₁-Y₂—, —(CH₂)ₚ-(4- to 12-heterocycloalkyl)-C(O)—Y₁—C(O)—Y₂—, —(CH₂)ₚ-(4- to 12-heterocycloalkyl)-Y₁-Y₂—, —(OCH₂CH₂)ₚ-(4- to 12-heterocycloalkyl)-Y₂—, —(CH₂OCH₂C(O))ₚ-(4- to 12-heterocycloalkyl)-Y₂, —(CH₂OCH₂C(O)NH)ₚ—Y₂—, —(CH₂)ₚ-(4- to 12-heterocycloalkyl)-Y₁—(CH₂CH₂O)ₚ—C(O)—Y₂—, —(CH₂)ₚ-(4- to 12-heterocycloalkyl)-Y₁—(CH₂CH₂O)ₚ—Y₂—, —(CH₂)ₚ-(4- to 12-heterocycloalkyl)-Y₁—(OCH₂CH₂)ₚ—Y₂—, —(CH₂)ₚ-(4- to 12-heterocycloalkyl)-Y₁—(OCH₂CH₂)ₚ—C(O)—(OCH₂CH₂)ₚ—Y₂—, —(CH₂OCH₂)ₚ-(heteroaryl)-Y₂—, —NHC(O)-(4- to 12-heterocycloalkyl)-Y₁—C(O)—Y₂—, —NHC(O)—(CH₂)ₚ-(4- to 12-heterocycloalkyl)-Y₁—C(O)—Y₂—, —NHC(O)—(CH₂)ₚ-(4- to 12-heterocycloalkyl)-Y₂—, —C(O)NH—Y₁-Y₂—;
   —(CH₂)ₚ—NH—Y₁-Y₂—;
   —C(O)NH—(CH₂CH₂O)ₚ—Y₂—,
   —C(O)—(CH₂CH₂O)ₚ—Y₂—,
   —(CH₂OCH₂)ₚ—NR₃C(O)—(CH₂CH₂O)ₚ—Y₂—,
   —(CH₂)ₚ—NR₃C(O)—(CH₂CH₂O)ₚ—Y₂—,
   —(CH₂OCH₂CH₂)ₚ-(4- to 12-heterocycloalkyl)-Y₁—C(O)—(CH₂CH₂O)ₚ—Y₂—
   —(CH₂OCH₂)ₚ—C(O)NH—(CH₂CH₂O)ₚ—Y₂—,
   wherein
      R₃ is —H or —C₁-C₃ alkyl,
      Y₁ is independently a bond, —C₁₋₈ alkylenyl, 4- to 12-membered heterocycloalkyl, or —C₃-C₁₀ cycloalkyl;
      Y₂ is independently
         a bond,
         —C₁-C₃ alkyl-,
         —(CH₂)ₘ—O—,
         —O—(CH₂)ₘ-(4- to 6-membered heterocycloalkyl),
         —(CH₂)ₘ—O—(CH₂)ₘ—NH—,
         —(CH₂)ₘ—NH—,
         —(CH₂)ₘ—NHC(O)—(CH₂)ₘO—,
         —(CH₂)ₘ—C(O)NH—(CH₂)ₘ—,
         —(CH₂)ₘ—C₃-C₁₀ cycloalkyl-,
         —(CH₂)ₘ-aryl,
         —(CH₂)ₘ-heteroaryl-,
         —(CH₂)ₘ-(4- to 6-membered heterocycloalkyl)-,
         —(CH₂)ₘ-(4- to 6-membered heterocycloalkyl)-C(O)—,
         —(CH₂)ₘ-(4- to 6-membered heterocycloalkyl)-C(O)—(CH₂)ₘ—,
         —(CH₂)ₘ-(4- to 6-membered heterocycloalkyl)-C(O)—(CH₂)ₘ—O—,
         —(CH₂)ₘ—C₃-C₁₀ cycloalkyl-C(O)NH—,
         —(CH₂)ₘ-(4- to 6-membered heterocycloalkyl)-O—,
X₁ is CH or N;
X₂ is CH or N;
m is independently at each occurrence an integer from 0 to 16;
n is an integer from 0 to 2; and
p is an integer from 0 to 3.

Another aspect of the instant disclosure is directed to compounds of Formula II:

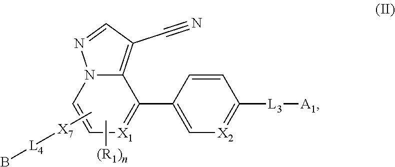

and pharmaceutically acceptable salts, solvates, isomers, enantiomers, and tautomers thereof,
wherein
R₁ is —OCH₂CH₃, —OCH₃, —OCF₃, —OCH₂CH₂OH, —OCH₂CH₂F, 1-methylpyrazol-4-yl, or halogen;
A₁ is independently aryl, or heteroaryl, wherein the aryl or heteroaryl is optionally substituted with one or more R₂; wherein R₂ is independently halogen, OH, C₁-C₃ alkyl, or C₁-C₃ alkoxy;
B is a moiety that is capable of binding a E3 ligase;
L₄ is independently —(CH₂)ₚ-(4- to 12-heterocycloalkyl)-Y₁—C(O)—Y₂—, —(CH₂)ₚ-(4- to 12-heterocycloalkyl)-C(O)—Y₁-Y₂—, —(CH₂)ₚ-(4- to 12-heterocycloalkyl)-Y₁-Y₂—,
   wherein
      Y₁ is independently —C₁₋₈ alkylenyl, 4- to 12-membered heterocycloalkyl, or —C₃-C₁₀ cycloalkyl;
      Y₂ is independently
         —C₁-C₃ alkyl-,
         —(CH₂)ₘ—O—,
         —O—(CH₂)ₘ-(4- to 6-membered heterocycloalkyl),
         —(CH₂)ₘ-(4- to 6-membered heterocycloalkyl)-,
         —(CH₂)ₘ-(4- to 6-membered heterocycloalkyl)-C(O)—, or
         —(CH₂)ₘ-(4- to 6-membered heterocycloalkyl)-O—,
L₃ is a bond, —C₁₋₈ alkylenyl, 4- to 8-membered heterocycloalkyl, —C₃-C₁₀ cycloalkyl, or -(4- to 8-membered heterocycloalkyl) (CH₂)ₘ—;
X₁ is CH or N;
X₂ is CH or N;
X₇ is O, S, NH, or a bond;
m is independently at each occurrence an integer from 0 to 16;
n is an integer from 0 to 2; and
p is an integer from 0 to 3.

Another aspect of the disclosure relates to a method of treating RET-mediated disorders and diseases in a subject in need thereof, comprising administering to the subject an effective amount of a compound of Formula I or Formula II.

Another aspect of the disclosure is directed to a method of modulating a RET protein. The method involves administering to a patient in need thereof an effective amount of a compound of Formula I or Formula II. In some embodiments, the method of treating RET mediated disorders and diseases in a subject includes modulating RET protein in the subject.

Another aspect of the disclosure is directed to pharmaceutical compositions comprising a compound of Formula I or Formula II and a pharmaceutically acceptable carrier. The pharmaceutically acceptable carrier can further include an excipient, diluent, or surfactant. The pharmaceutical composition can be effective for treating a disease or disorder associated with RET modulation in a subject in need thereof. The pharmaceutical compositions can comprise the compounds of the present invention for use in treating diseases described herein. The compositions can contain at least one compound of the invention and a pharmaceutically acceptable carrier.

Another aspect of the present disclosure relates to a compound of Formula I or Formula II, or a pharmaceutically acceptable salt, hydrate, solvate, prodrug, stereoisomer, or tautomer thereof, for use in treating or preventing a disease associated with RET modulation. The invention also provides the use of the compounds described herein in the manufacture of a medicament for the treatment of a disease associated with RET.

The present invention also provides methods for the treatment of diseases or disorders by degrading RET the using the instant compounds of Formula I or Formula II and pharmaceutically acceptable composition of the compounds of Formula I or Formula II. The present invention also provides compounds of Formula I or Formula II that bind and inhibit to E3 ligase and further degrade the RET protein. These E3 ligase-binding compounds (degraders) can also be useful in the treatment of RET mediated diseases and cancer including Hirschsprung disease, medullary thyroid cancer (MTC), thyroid carcinoma, familial medullary thyroid carcinoma, multiple endocrine neoplasia, multiple endocrine neoplasia type 2 (MEN-2, MEN-2A, MEN-2B), neuroendocrine tumors, central nervous system tumors, central hypoventilation syndrome, renal agenesis, pheochromocytoma, parathyroid hyperplasia, and non-small cell lung cancer.

The present invention further provides compounds that can degrade RET while binding E3 ligase. In some embodiments, the efficacy-safety profile of the compounds of the current disclosure can be improved relative to other known RET inhibitors. The compound of the present invention can provide multiple advantages over traditional treatments with a RET binding ligand, such as overcome resistance in certain cases and prolong the kinetics of drug effect by destroying the protein.

Additionally, the present technology also has the advantage of being able to be used for a number of different types of diseases, including disorder mediated by RET fusion proteins, overexpression, or copy number gains, such as papillary thyroid cancer, pancreatic cancer, melanoma, leukemia, acute myeloid leukemia (AML), chronic myelomonocytic leukemia, lung adenocarcinomas, lung cancer, non-small cell lung cancer (NSCLC), nonsyndromic paraganglioma, breast cancer, nonhereditary (sporadic) cancers, colorectal, or a hematologic malignancy. Additional features and advantages of the present technology will be apparent to one of skill in the art upon reading the Detailed Description of the Invention, below.

### DETAILED DESCRIPTION OF THE INVENTION

The present disclosure relates to RET-degrading compounds of Formula I or Formula II, pharmaceutically acceptable salts, solvates, hydrates, isomer, and tautomer thereof, which may inhibit E3 ligase activity and are accordingly useful in methods of treatment of the human or animal body. The present disclosure also relates to processes for the preparation of these compounds, to pharmaceutical compositions comprising them and to their use in the treatment of disorders and diseases in which RET is implicated, such as inflammation, an auto-immune disease, a cancer, an infection, a disease or disorder of the central nervous system, a metabolic disease, a cardiovascular disease, a respiratory disease, a kidney disease, a liver disease, an ocular disease, a skin disease, a lymphatic disease, a rheumatic disease, a psychological disease, graft versus host disease, allodynia, or an RET-related disease in a subject that has been determined to carry a germline or somatic mutation in RET.

### DEFINITIONS

Unless otherwise stated, the following terms used in the specification and claims have the following meanings set out below.

As used herein, “Alkyl” refers to optionally substituted, straight and branched chain aliphatic groups having from 1 to 30 carbon atoms. “C1, C2, C3, C4, C5 or C6 alkyl” or “C1-C6 alkyl” is intended to include C1, C2, C3, C4, C5 or C6 straight chain (linear) saturated aliphatic hydrocarbon groups and C3, C4, C5 or C6 branched saturated aliphatic hydrocarbon groups. For example, C1-C6 alkyl is intends to include C1, C2, C3, C4, C5 and C6 alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl, or n-hexyl. In some embodiments, a straight chain or branched alkyl has six or fewer carbon atoms (e.g., C1-C6 for straight chain, C3-C6 for branched chain), and in another embodiment, a straight chain or branched alkyl has four or fewer carbon atoms. The term “heteroalkyl” as used herein contemplates an alkyl with one or more heteroatoms.

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g., C2-C6 for straight chain, C3-C6 for branched chain). The term “C2-C6” includes alkenyl groups containing two to six carbon atoms. The term “C3-C6” includes alkenyl groups containing three to six carbon atoms.

As used herein, the term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

As used herein, the term “alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g., C2-C6 for straight chain, C3-C6 for branched chain). The term “C2-C6” includes alkynyl groups containing two to six carbon atoms. The term “C3-C6” includes alkynyl groups containing three to six carbon atoms. As used herein, “C2-C6 alkenylene linker” or “C2-C6 alkynylene linker” is intended to include C2, C3, C4, C5 or C6 chain (linear or branched) divalent unsaturated aliphatic hydrocarbon groups. For example, C2-C6 alkenylene linker is intended to include C2, C3, C4, C5 and C6 alkenylene linker groups.

As used herein, the term “optionally substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

As used herein, the term “cyano” refers to a nitrile radical (e.g., —CN).

As used herein, the term “cycloalkyl” refers to a saturated or partially unsaturated hydrocarbon monocyclic or polycyclic (e.g., fused, bridged, or spiro rings) system having 3 to 30 carbon atoms (e.g., C3-C12, C3-C10, or C3-C8). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. In the case of polycyclic cycloalkyl, only one of the rings in the cycloalkyl needs to be non-aromatic.

As used herein, the term “heterocycloalkyl” refers to a saturated or partially unsaturated 3-8 membered monocyclic or bicyclic, 7-12 membered bicyclic (fused, bridged, or spiro rings), or 11-14 membered tricyclic ring system (fused, bridged, or spiro rings) having one or more heteroatoms (such as O, N, S, P, or Se), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofurran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, 5,6-dihydro-4H-cyclopenta[b]thiophenyl, and the like. In the case of multicyclic heterocycloalkyl, only one of the rings in the heterocycloalkyl needs to be non-aromatic (e.g., 4,5,6,7-tetrahydrobenzo[c]isoxazolyl).

As used herein, the term “optionally substituted heterocycloalkyl” refers to unsubstituted heterocycloalkyl having designated substituents replacing one or more hydrogen atoms on one or more carbon or heteroatom. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Unless otherwise specifically defined, the term “aryl” refers to cyclic, aromatic hydrocarbon groups that have 1 to 3 aromatic rings, including monocyclic or bicyclic groups such as phenyl, biphenyl, or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. Exemplary substituents include, but are not limited to, —H, -halogen. —O—(C1-C6) alkyl, (C1-C6) alkyl, —O—(C2-C6) alkenyl, —O—(C2-C6)alkynyl, (C2-C6)alkenyl, (C2-C6)alkynyl, —OH, —OP(O)(OH)2, —OC(O)(C1-C6)alkyl, —C(O)(C1-C6)alkyl, —OC(O)O(C1-C6)alkyl, —NH2, NH((C1-C6) alkyl), N((C1-C6)alkyl)2, —S(O)2-(C1-C6)alkyl, —S(O)NH(C1-C6)alkyl, and —S(O)N((C1-C6)alkyl)2. The substituents can themselves be optionally substituted. Furthermore, when containing two or more fused rings, the aryl groups herein defined may have a saturated or partially unsaturated ring fused with a fully unsaturated aromatic ring Exemplary ring systems of these aryl groups include, but are not limited to, phenyl, biphenyl, naphthyl, anthracenyl, phenalenyl, phenanthrenyl, indanyl, indenyl, tetrahydronaphthalenyl, tetrahydrobenzoannulenyl, 10,11-dihydro-5H-dibenzo[a,d][7]annulenyl, and the like.

Unless otherwise specifically defined, “heteroaryl” means a monovalent monocyclic or polycyclic aromatic radical of 5 to 24 ring atoms, containing one or more ring heteroatoms selected from N, O, S, P, Se, or B, the remaining ring atoms being C. Heteroaryl as herein defined also means a bicyclic heteroaromatic group wherein the heteroatom is selected from N, O, S, P, Se, or B. Heteroaryl as herein defined also means a tricyclic heteroaromatic group containing one or more ring heteroatoms selected from N, O, S, P, Se, or B. The aromatic radical is optionally substituted independently with one or more substituents described herein. Examples include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, thiophen-2-yl, quinolinyl, benzopyranyl, isothiazolyl, thiazolyl, thiadiazole, indazole, benzimidazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydro pyrrolo[1,2-a]pyrimidinyl, 3,4-dihydro-2H-1λ2-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, benzoxazolyl, benzisoxazolyl, furo[2,3-b]pyridinyl, benzothiophenyl, 1,5-naphthyridinyl, furo[3,2-b]pyridine, [1,2,4]triazolo[1,5-a]pyridinyl, benzo[1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo[1,5-b][1,2]oxazinyl, 4,5,6,7-tetrahydropyrazolo[1,5-a]pyridinyl, thiazolo[5,4-d]thiazolyl, imidazo[2,1-b][1,3,4]thiadiazolyl, thieno[2,3-b]pyrrolyl, 3H-indolyl, and derivatives thereof. Furthermore, when containing two or more fused rings, the heteroaryl groups defined herein may have one or more saturated or partially unsaturated ring fused with a fully unsaturated aromatic ring, e.g., a 5-membered heteroaromatic ring containing 1 to 3 heteroatoms selected from N, O, S, P, Se, or B, or a 6-membered heteroaromatic ring containing 1 to 3 nitrogens, wherein the saturated or partially unsaturated ring includes 0 to 4 heteroatoms selected from N, O, S, P, Se, or B, and is optionally substituted with one or more oxo. In heteroaryl ring systems containing more than two fused rings, a saturated or partially unsaturated ring may further be fused with a saturated or partially unsaturated ring described herein. Exemplary ring systems of these heteroaryl groups include, for example, indolinyl, indolinonyl, dihydrobenzothiophenyl, dihydrobenzofuran, chromanyl, thiochromanyl, tetrahydroquinolinyl, dihydrobenzothiazine, 3,4-dihydro-11H-isoquinolinyl, 2,3-dihydrobenzofuranyl, benzofuranonyl, indolinyl, oxindolyl, indolyl, 1,6-dihydro-7H-pyrazolo[3,4-c]pyridin-7-onyl, 7,8-dihydro-6H-pyrido[3,2-b]pyrrolizinyl, 8H-pyrido[3,2-b]pyrrolizinyl, 1,5,6,7-tetrahydrocyclopenta[b]pyrazolo[4,3-e]pyridinyl, 7,8-dihydro-6H-pyrido[3,2-b]pyrrolizine, pyrazolo[1,5-a]pyrimidin-7(4H)-only, 3,4-dihydropyrazino[1,2-a]indol-1(2H)-onyl, or benzo[c][1,2]oxaborol-1(3H)-olyl.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl).

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2R moieties, then the group may optionally be substituted with up to two R moieties and R at each occurrence is selected independently from the definition of R. Also, combinations of substituents and/or variables are permissible, but only if such combinations result in stable compounds.

As used herein, the term “hydroxy” or “hydroxyl” includes groups with an —OH or —O—.

As used herein, the term “halo” or “halogen” refers to fluoro, chloro, bromo and iodo.

The term “haloalkyl” or “haloalkoxyl” refers to an alkyl or alkoxyl substituted with one or more halogen atoms.

As used herein, the term “optionally substituted haloalkyl” refers to unsubstituted haloalkyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

As used herein, the term “substituted,” means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a RM, and formulation into an efficacious therapeutic agent.

It is to be understood that the present disclosure provides methods for the synthesis of the compounds of any of the Formulae described herein. The present disclosure also provides detailed methods for the synthesis of various disclosed compounds of the present disclosure according to the following schemes as well as those shown in the Examples.

It is to be understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

It is to be understood that the synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

It is to be understood that compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5th edition, John Wiley & Sons: New York, 2001; Greene, T. W., Wuts, P. G. M., Protective Groups in Organic Synthesis, 3rd edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed, Encyclopedia of Reagents for Organic Synthesis, John Wiley and Sons (1995), incorporated by reference herein, are useful and recognised reference textbooks of organic synthesis known to those in the art.

One of ordinary skill in the art will note that, during the reaction sequences and synthetic schemes described herein, the order of certain steps may be changed, such as the introduction and removal of protecting groups. One of ordinary skill in the art will recognise that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3rd edition. John Wiley & Sons. New York, 1999.

It is to be understood that, unless otherwise stated, any description of a method of treatment or prevention includes use of the compounds to provide such treatment or prevention as is described herein. It is to be further understood, unless otherwise stated, any description of a method of treatment or prevention includes use of the compounds to prepare a medicament to treat or prevent such condition. The treatment or prevention includes treatment or prevention of human or non-human animals including rodents and other disease models.

It is to be understood that, unless otherwise stated, any description of a method of treatment includes use of the compounds to provide such treatment as is described herein. It is to be further understood, unless otherwise stated, any description of a method of treatment includes use of the compounds to prepare a medicament to treat such condition. The treatment includes treatment of human or non-human animals including rodents and other disease models. As used herein, the term “subject” is interchangeable with the term “subject in need thereof”, both of which refer to a subject having a disease or having an increased risk of developing the disease. A “subject” includes a mammal. The mammal can be e.g., a human or appropriate non-human mammal, such as primate, mouse, rat, dog, cat, cow, horse, goat, camel, sheep or a pig. In one embodiment, the mammal is a human. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model. It is to be appreciated that references to “treating” or “treatment” include the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

It is to be understood that a compound of the present disclosure, or a pharmaceutically acceptable salt, polymorph or solvate thereof, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005), Sambrook et al., Molecular Cloning, A Laboratory Manual 3rd edition), Cold Spring Harbor Press, Cold Spring Harbor, N.Y. (2000); Coligan et al., Current Protocols in Immunology, John Wiley & Sons, N Y.: Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, Pa., 18th edition (1990). These texts can, of course, also be referred to in making or using an aspect of the disclosure.

It is to be understood that the present disclosure also provides pharmaceutical compositions comprising any compound described herein in combination with at least one pharmaceutically acceptable excipient or carrier.

As used herein, the term “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

It is to be understood that a pharmaceutical composition of the disclosure is formulated to be compatible with its intended route of administration. Examples of routes of administration include parenteral, e.g., intravenous, intradermal, subcutaneous, oral (e.g., ingestion), inhalation, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens, antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

It is to be understood that a compound or pharmaceutical composition of the disclosure can be administered to a subject in many of the well-known methods currently used for chemotherapeutic treatment. For example, a compound of the disclosure may be injected into the blood stream or body cavities or taken orally or applied through the skin with patches. The dose chosen should be sufficient to constitute effective treatment but not so high as to cause unacceptable side effects. The state of the disease condition (e.g., a disease or disorder disclosed herein) and the health of the patient should preferably be closely monitored during and for a reasonable period after treatment.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

It is to be understood that, for any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilising processes Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilisation. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebuliser.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated, each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the symptoms of the disease or disorder disclosed herein and also preferably causing complete regression of the disease or disorder. Dosages can range from about 0.01 mg/kg per day to about 5000 mg/kg per day. An effective amount of a pharmaceutical agent is that which provides an objectively identifiable improvement as noted by the clinician or other qualified observer. Improvement in survival and growth indicates regression. As used herein, the term “dosage effective manner” refers to amount of an active compound to produce the desired biological effect in a subject or cell.

It is to be understood that the pharmaceutical compositions can be included in a container, pack, or dispenser together with instructions for administration.

It is to be understood that, for the compounds of the present disclosure being capable of further forming salts, all of these forms are also contemplated within the scope of the claimed disclosure.

As used herein, the term “pharmaceutically acceptable salts” refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

In some embodiments, the pharmaceutically acceptable salt is a sodium salt, a potassium salt, a calcium salt, a magnesium salt, a diethylamine salt, a choline salt, a meglumine salt, a benzathine salt, a tromethamine salt, an ammonia salt, an arginine salt, or a lysine salt.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion, or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ratio other than 1:1, e.g., 3:1, 2:1, 1:2, or 1:3.

It is to be understood that all references to pharmaceutically acceptable salts include solvent addition forms (solvates) or crystal forms (polymorphs) as defined herein, of the same salt.

The compounds, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In one embodiment, the compound is administered orally. One skilled in the art will recognise the advantages of certain routes of administration.

The dosage regimen utilising the compounds is selected in accordance with a variety of factors including type, species, age, weight, sex and medical condition of the patient; the severity of the condition to be treated, the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition. An ordinarily skilled physician or veterinarian can readily determine and prescribe the effective amount of the drug required to counter or arrest the progress of the condition.

Techniques for formulation and administration of the disclosed compounds of the disclosure can be found in Remington: the Science and Practice of Pharmacy, 19th edition, Mack Publishing Co, Easton, Pa. (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers, however, it will be understood that a given isomer, tautomer, regioisomer or stereoisomer may have a higher level of activity than another isomer, tautomer, regioisomer or stereoisomer.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

As use herein, the phrase “compound of the disclosure”, “degraders of the disclosure”, “degraders”, refers to those compounds which are disclosed herein, both generically and specifically.

### Compounds of the Disclosure

In one aspect, the present disclosure provides, inter alia, compounds of Formula (I):

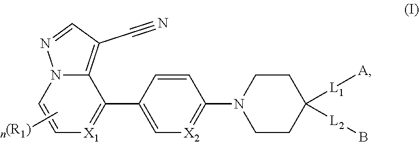

and pharmaceutically acceptable salts, solvates, hydrates, isomers or tautomers thereof, wherein A, B, L₁, L₂, R₁, X₁, X₂, and n are described herein as set forth in the disclosure.

In some embodiments, the disclosure relates to compounds of Formula I(a):

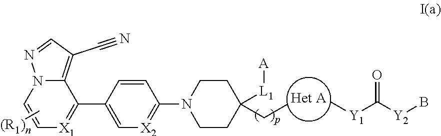

wherein Het A is 4- to 6-membered heterocycloalky.

In other embodiments, the compounds of the disclosure are of Formula I(b):

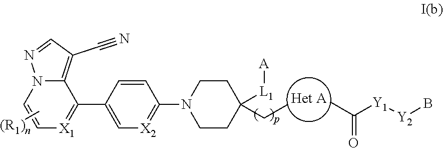

wherein Het A is 4- to 6-membered heterocycloalky.

In other embodiments, the compounds of the disclosure are of Formula I(c):

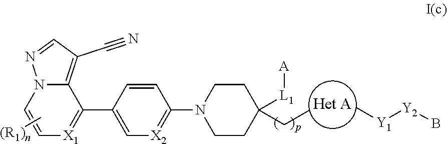

wherein Het A is 4- to 6-membered heterocycloalky.

In other embodiments, the compounds of the disclosure are of Formula I(d):

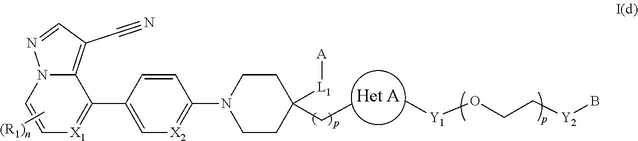

wherein Het A is 4- to 6-membered heterocycloalky.

In other embodiments, the compounds of the disclosure are of Formula I(e):

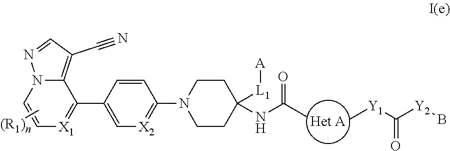

wherein Het A is 4- to 6-membered heterocycloalky.

In yet other embodiments, the compounds of the disclosure are of Formula I(f):

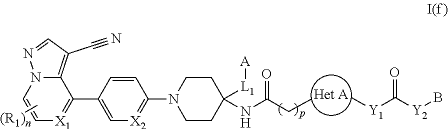

wherein Het A is 4- to 6-membered heterocycloalky.

In other embodiments, the compounds of the disclosure are of Formula I(g):

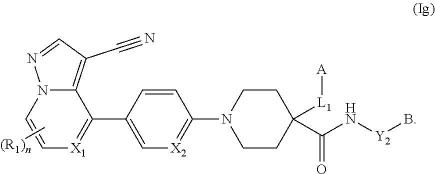

In other embodiments, the compounds of the disclosure are of Formula I(h):

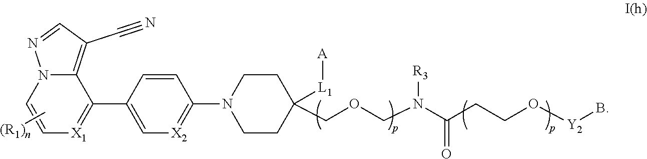

In other embodiments, the compounds of the disclosure are of Formula II:

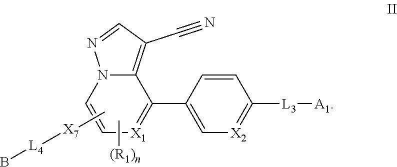

In some embodiments of the compounds of the disclosure, R₁ is halogen, —OCH₂CH₃, —OCF₃, —OCH₂CH₂OH, or —OCH₂CH₂F. In some embodiments, R₁ may be halogen, —OCH₂CH₃, —OCF₃, or —OCH₂CH₂OH. In some embodiments, R₁ may be halogen, —OCH₂CH₃, or —OCF₃. In some embodiments, R₁ may be halogen or —OCH₂CH₃. In some embodiments, R₁ is halogen. In some embodiments, R₁ is —OCH₂CH₃. In some embodiments, R₁ is —OCF₃. In some embodiments, R₁ is —OCH₂CH₂OH. In some embodiments, R₁ is —OCH₃. In some embodiments, R₁ is 1-methyl-pyrazol-4-yl.

In certain embodiments of the compounds of the disclosure, A is H, OH, C₁-C₆ alkyl, C₃-C₈ cycloalkyl, aryl, or heteroaryl. In other embodiments, A is OH, C₁-C₆ alkyl, C₃-C₈ cycloalkyl, or aryl. In other embodiments, A is OH, C₁-C₆ alkyl, or C₃-C₈ cycloalkyl. In other embodiments, A is OH or C₁-C₆ alkyl. In other embodiments, A is OH. In other embodiments, A is C₁-C₆ alkyl. In other embodiments, A is C₃-C₈ cycloalkyl. In other embodiments, A is aryl. In other embodiments, A is heteroaryl. In other embodiments, A is C₁-C₆ alkyl optionally substituted with one or more R₂. In other embodiments, A is C₃-C₈ cycloalkyl optionally substituted with one or more R₂. In other embodiments, A is aryl optionally substituted with one or more R₂. In other embodiments, A is heteroaryl optionally substituted with one or more R₂.

In certain embodiments of the compounds of the disclosure, A₁ is aryl, or heteroaryl. In other embodiments, A is aryl optionally substituted with one or more R₂. In other embodiments, A is heteroaryl optionally substituted with one or more R₂.

In some embodiments, R₂ is halogen, OH, C₁-C₃ alkyl, or C₁-C₃ alkoxy. In other embodiments, R₂ is halogen. In other embodiments, R₂ is OH. In other embodiments, R₂ is C₁-C₃ alkyl. In other embodiments, R₂ is C₁-C₃ alkoxy. In other embodiments, A is H.

In some embodiments of the compounds of the disclosure L₁ is a bond, —C₁-C₃ alkylenyl-, —NHCO—, —CONH—, —O—, or —NH—. In further embodiments, L₁ may be —C₁-C₃ alkylenyl-, —NHCO—, —CONH—, or —O—. In further embodiments, L₁ may be —C₁-C₃ alkylenyl-, —NHCO—, or —CONH—. In further embodiments, L₁ may be —C₁-C₃ alkylenyl- or —NHCO—. In further embodiments, L₁ may be —C₁-C₃ alkylenyl-. Yet in further embodiments, L₁ may be —NHCO—. Yet in further embodiments, L₁ may be —CONH—. Yet, in further embodiments, L₁ may be —O—. Yet, in further embodiments, L₁ may be —NH—. Yet, in further embodiments, L₁ may be a bond.

In certain embodiments of the compounds of the disclosure, L₂ is independently —(CH₂)ₚ-(4- to 12-heterocycloalkyl)-Y₁—C(O)—Y₂—.

In certain embodiments, L₂ is —(CH₂)ₚ-(4- to 12-heterocycloalkyl)-C(O)—Y₁-Y₂—.

In some embodiments, L₂ is —(CH₂)ₚ-(4- to 12-heterocycloalkyl)-C(O)—Y₁—C(O)—Y₂—.

In some embodiments, L₂ is —(CH₂)ₚ-(4- to 12-heterocycloalkyl)-Y₁-Y₂—.

In certain embodiments, L₂ is —(OCH₂CH₂)ₚ-(4- to 12-heterocycloalkyl)-Y₂—.

In certain embodiments, L₂ is —(CH₂OCH₂C(O))ₚ-(4- to 12-heterocycloalkyl)-Y₂—.

In certain embodiments, L₂ is —(CH₂OCH₂C(O)NH)ₚ—Y₂—.

In some embodiments, L₂ is —(CH₂)ₚ-(4- to 12-heterocycloalkyl)-Y₁—(CH₂CH₂O)ₚ—C(O)—Y₂—.

In some embodiments, L₂ is —(CH₂)ₚ-(4- to 12-heterocycloalkyl)-Y₁—(CH₂CH₂O)ₚ—Y₂—.

In some embodiments, L₂ is —(CH₂)ₚ-(4- to 12-heterocycloalkyl)-Y₁—(OCH₂CH₂)ₚ—Y₂—.

In some embodiments, L₂ is —(CH₂)ₚ-(4- to 12-heterocycloalkyl)-Y₁—(OCH₂CH₂)ₚ—C(O)—(OCH₂CH₂)ₚ—Y₂—.

In certain embodiments, L₂ is —(CH₂OCH₂)ₚ-(heteroaryl)-Y₂—.

In certain embodiments, L₂ is —NHC(O)-(4- to 12-heterocycloalkyl)-Y₁—C(O)—Y₂—.

In certain embodiments, L₂ is —NHC(O)—(CH₂)ₚ-(4- to 12-heterocycloalkyl)-Y₁—C(O)—Y₂—.

In some embodiments, L₂ is —NHC(O)—(CH₂)ₚ-(4- to 12-heterocycloalkyl)-Y₂—.

In some embodiments, L₂ is —C(O)NH—Y₁-Y₂—.

In some embodiments, L₂ is —(CH₂)ₚ—NH—Y₁-Y₂—.

In certain embodiments, L₂ is —C(O)NH—(CH₂CH₂O)ₚ—Y₂—.

In certain embodiments, L₂ is —(CH₂OCH₂)ₚ—NR₃C(O)—(CH₂CH₂O)ₚ—Y₂—.

In some embodiments, L₂ is —(CH₂)ₚ—NR₃C(O)—(CH₂CH₂O)ₚ—Y₂—.

In some embodiments, L₂ is —(CH₂OCH₂CH₂)ₚ-(4- to 12-heterocycloalkyl)-Y₁—C(O)—(CH₂CH₂O)ₚ—Y₂—.

In some embodiments, L₂ is —(CH₂OCH₂)ₚ—C(O)NH—(CH₂CH₂O)ₚ—Y₂—.

In some embodiments, L₃ is a bond, —C₁₋₈ alkylenyl, 4- to 12-membered heterocycloalkyl, —C₃-C₁₀ cycloalkyl, or -(4- to 8-membered heterocycloalkyl)-(CH₂)ₘ—. In some embodiments, L₃ is a bond. In some embodiments, L₃ is —C₁₋₈ alkylenyl. In some embodiments, L₃ is 4- to 8-membered heterocycloalkyl. In some embodiments, L₃ is —C₃-C₁₀ cycloalkyl. In some embodiments, L₃ is -(4- to 8-membered heterocycloalkyl) (CH₂)ₘ—.

In certain embodiments of the compounds of the disclosure, L₄ is —(CH₂)ₚ-(4-to 12-heterocycloalkyl)-Y₁—C(O)—Y₂—.

In certain embodiments of the compounds of the disclosure, L₄ is —(CH₂)ₚ-(4-to 12-heterocycloalkyl)-C(O)—Y₁-Y₂—.

In certain embodiments of the compounds of the disclosure, L₄ is —(CH₂)ₚ-(4-to 12-heterocycloalkyl)-Y₁-Y₂—.

In a further embodiment of the compounds of the instant disclosure, R₃ is —H or —C₁-C₃ alkyl. In yet another embodiment, R₃ is —H. In yet another embodiment, R₃ is —C₁-C₃ alkyl.

In some embodiments of the disclosure, Y₁ is a bond, —C₁₋₈ alkylenyl, 4- to 12-membered heterocycloalkyl, or —C₃-C₁₀ cycloalkyl. In some embodiments, Y₁ is a single bond. In other embodiments, Y₁ is C₁₋₈ alkylenyl. In other embodiments, Y₁ is 4- to 12-membered heterocycloalkyl. In other embodiments, Y₁ is —C₃-C₁₀ cycloalkyl.

In further embodiments of the compounds of the disclosure, Y₂ may be a single bond.

In a further embodiment of the disclosure, Y₂ is —C₁-C₃ alkyl-.

In yet another embodiment of the disclosure, Y₂ is —(CH₂)ₘ—O—.

In other embodiments of the disclosure, Y₂ is —O—(CH₂)ₘ-(4- to 6-membered heterocycloalkyl)-.

In other embodiments of the disclosure, Y₂ is —(CH₂)ₘ—O—(CH₂)ₘ—NH—.

In other embodiments of the disclosure, Y₂ is —(CH₂)ₘ—NH—.

In other embodiments of the disclosure, Y₂ is —(CH₂)ₘ—NHC(O)—(CH₂)ₘO—.

In certain embodiments of the disclosure, Y₂ is —(CH₂)ₘ—C(O)NH—(CH₂)ₘ—.

In certain embodiments of the disclosure, Y₂ is —(CH₂)ₘ—C₃-C₁₀ cycloalkyl-.

In certain embodiments of the disclosure, Y₂ is —(CH₂)ₘ-aryl-.

In certain embodiments of the disclosure, Y₂ is —(CH₂)ₘ-heteroaryl-.

In certain embodiments of the disclosure, Y₂ is —(CH₂)ₘ-(4- to 6-membered heterocycloalkyl)-.

In certain embodiments of the disclosure, Y₂ is —(CH₂)ₘ-(4- to 6-membered heterocycloalkyl)-C(O)—.

In certain embodiments of the disclosure, Y₂ is —(CH₂)ₘ-(4- to 6-membered heterocycloalkyl)-C(O)—(CH₂)ₘ—.

In other embodiments of the disclosure, Y₂ is —(CH₂)ₘ-(4- to 6-membered heterocycloalkyl) C(O)—(CH₂)ₘ—O—.

In other embodiments of the disclosure, Y₂ is —(CH₂)ₘ—C₃-C₁₀ cycloalkyl-C(O)NH—.

In other embodiments of the disclosure, Y₂ is —(CH₂)ₘ-(4- to 6-membered heterocycloalkyl)-O—.

In other embodiments of the disclosure, X₁ is CH or N. In some embodiments, X₁ is CH. In some embodiments, X₁ is N.

In other embodiments of the disclosure, X₂ is CH or N. In some embodiments, X₂ is CH. In some embodiments, X₂ is N.

In other embodiments of the disclosure, X₇ is O, S, NH, or a bond. In some embodiments, X₇ is O. In some embodiments, X₇ is S. In some embodiments, X₇ is NH. In some embodiments, X₇ is a bond.

In further embodiments of the compounds of the instant disclosure, m is at each occurrence, 0, 1, 2, 3, 4, 5, 6, 7, 8, 9, 10, 11, 12, 13, 14, 15, or 16. In some embodiments, m is independently 0. In some embodiments, m is independently 1. In some embodiments, m is independently 2. In some embodiments, m is independently 3. In some embodiments, m is independently 4. In some embodiments, m is independently 5. In some embodiments, m is independently 6. In some embodiments, m is independently 7. In some embodiments, m is independently 8. In some embodiments, m is independently 9. In some embodiments, m is independently 10. In some embodiments, m is independently 11. In some embodiments, m is independently 2. In some embodiments, m is independently 13. In some embodiments, m is independently 14. In some embodiments, m is independently 15. In some embodiments, m is independently 16.

In yet other embodiments of the disclosure, n is, independently at each occurrence, 0, 1, or 2. In yet other embodiments, n is 0 or 1. In yet other embodiments, n is 0 or 2. In yet other embodiments, n is 1 or 2. In yet other embodiments, n is 0. In yet other embodiments, n is 1. In yet other embodiments, n is 2.

In further embodiments of the disclosure, p is, independently at each occurrence, 0, 1, 2, or 3. In yet other embodiments, p is 1 or 2. In yet other embodiments, p is 1 or 3. In yet other embodiments, p is 2 or 3. In yet other embodiments, p is 1. In yet other embodiments, p is 2. In yet other embodiments, p is 3. In a further embodiment, p is 0.

In yet further embodiments of the disclosure, B represents a moiety that is capable of binding a E3 ligase. In some embodiments, the E3 ligase binding portion B is of the structure:

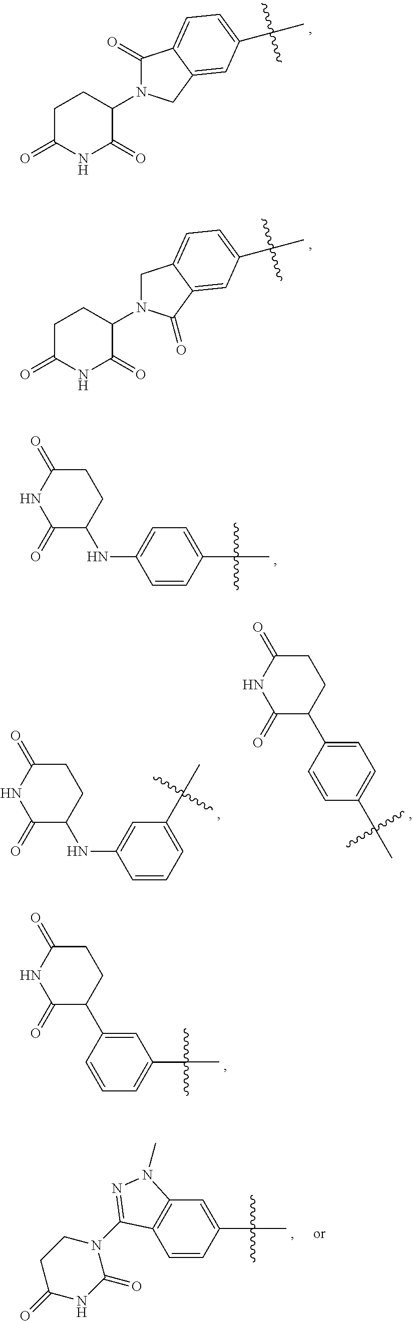

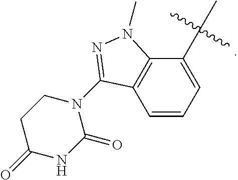 [SMILES: *c1cccc2c(N3CCC(=O)NC3=O)nn(C)c12]

In other embodiments, the E3 ligase binding portion B is of the structure:

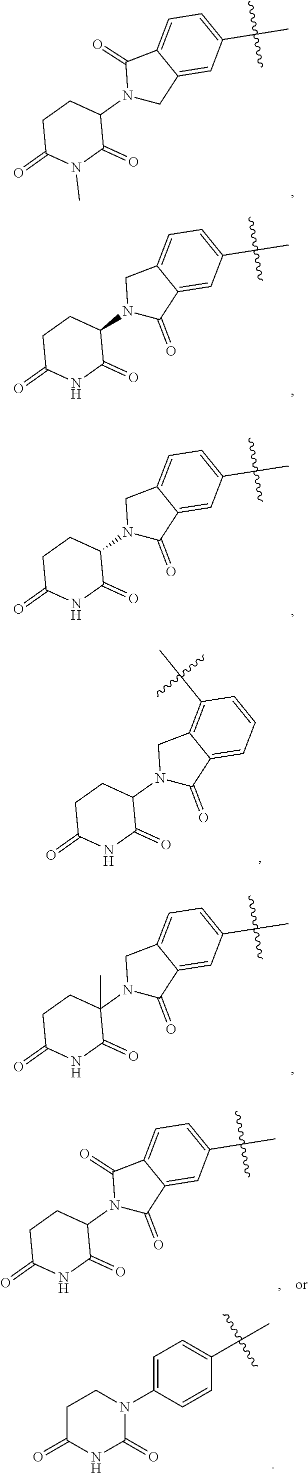

In other embodiments, E3 ligase binding portion B is cereblon subunit. In further embodiments, the cereblon subunit may be of the general structure:

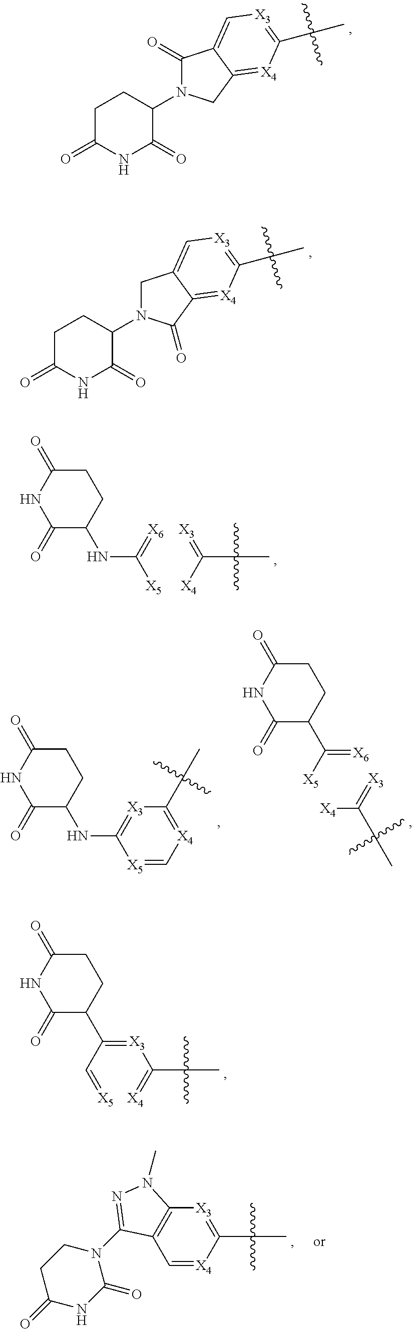

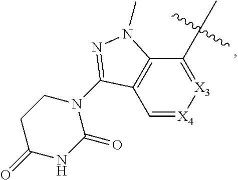

wherein X₃, X₄, X₅, and X₆ are independently N or CH.

In further embodiments, the cereblon subunit may be of the general structure:

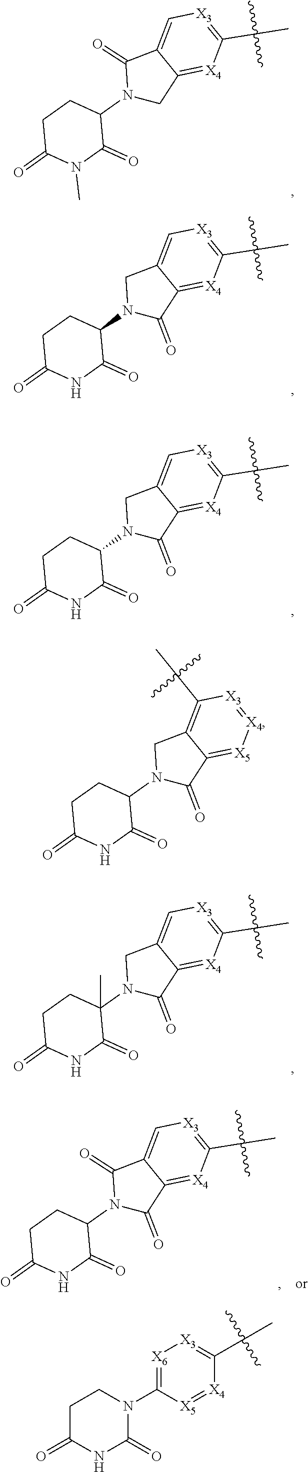

wherein X₃, X₄, X₅, and X₆ are independently N or CH.

In other embodiments, suitable compounds of the disclosure include:
N-(1-(5-(3-cyano-6-ethoxypyrazolo[1,5-a]pyridin-4-yl)pyridin-2-yl)-4-((4-(7-(4-(2-(2,6-dioxopiperidin-3-yl)-1-oxoisoindolin-5-yl)piperazin-1-yl)heptanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(7-(4-(2-(2,6-dioxopiperidin-3-yl)-1-oxoisoindolin-5-yl)piperazin-1-yl)heptanoyl)piperazin-1-yl)methyl)piperidin-4-yl)-3-fluoropicolinamide;
N-(1-(5-(3-cyano-6-ethoxypyrazolo[1,5-a]pyridin-4-yl)pyridin-2-yl)-4-((4-(7-(4-(2-(2,6-dioxopiperidin-3-yl)-1-oxoisoindolin-5-yl)piperazin-1-yl)heptanoyl)piperazin-1-yl)methyl)piperidin-4-yl)-3-methylbutanamide;
N-(1-(5-(3-cyano-6-ethoxypyrazolo[1,5-a]pyridin-4-yl)pyridin-2-yl)-4-((4-(3-(4-(2-(2,6 dioxopiperidin-3-yl)-3-oxoisoindolin-5-yl)piperidin-1-yl)propanoyl)piperazin-1-yl)methyl)piperidin-4-yl)-3-fluoropicolinamide;
N-(1-(5-(3-cyano-6-ethoxypyrazolo[1,5-a]pyridin-4-yl)pyridin-2-yl)-4-((4-(5-(4-(2-(2,6-dioxopiperidin-3-yl)-1-oxoisoindolin-5-yl)piperazin-1-yl)pentanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(2-(4-(2-(2,6-dioxopiperidin-3-yl)-1-oxoisoindolin-5-yl)piperazin-1-yl)acetyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(7-(4-(2-(2,6-dioxopiperidin-3-yl)-1-oxoisoindolin-5-yl)piperazin-1-yl)heptanoyl)piperazin-1-yl)methyl)piperidin-4-yl)cyclopropanecarboxamide;
N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)propanoyl)piperazin-1-yl)methyl)piperidin-4-yl)-3-methylbutanamide;
N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)propanoyl)piperazin-1-yl)methyl)piperidin-4-yl)cyclopropanecarboxamide;
N-(1-(5-(3-cyano-6-ethoxypyrazolo[1,5-a]pyridin-4-yl)pyridin-2-yl)-4-((4-(5-(4-(2-(2,6-dioxopiperidin-3-yl)-1-oxoisoindolin-5-yl)piperidin-1-yl)pentanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(7-(4-(2-(2,6-dioxopiperidin-3-yl)-1-oxoisoindolin-5-yl)piperidin-1-yl)heptanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperazin-1-yl)propanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(5-(4-(2-(2,6-dioxopiperidin-3-yl)-3-oxoisoindolin-5-yl)piperazin-1-yl)pentanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(4-(4-(2-(2,6-dioxopiperidin-3-yl)-1-oxoisoindolin-5-yl)piperazin-1-yl)butanoyl)piperazin-1-yl)methyl)piperidin-4-yl)-2,5-difluorobenzamide;
N-[1-(5-{3-cyano-6-ethoxypyrazolo[1,5-a]pyridin-4-yl}pyridin-2-yl)-4-{[4-(3-{4-[2-(2,6-dioxopiperidin-3-yl)-1-oxo-2,3-dihydro-1H-isoindol-5-yl]piperidin-1-yl}propanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(7-{4-[2-(2,6-dioxopiperidin-3-yl)-3-oxo-2,3-dihydro-1H-isoindol-5-yl]piperazin-1-yl}heptanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(3-{4-[2-(2,6-dioxopiperidin-3-yl)-3-oxo-2,3-dihydro-1H-isoindol-5-yl]piperidin-1-yl}propanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(5-{4-[2-(2,6-dioxopiperidin-3-yl)-3-oxo-2,3-dihydro-1H-isoindol-5-yl]piperidin-1-yl}pentanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(7-{4-[2-(2,6-dioxopiperidin-3-yl)-3-oxo-2,3-dihydro-1H-isoindol-5-yl]piperidin-1-yl}heptanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(7-{[4-(2,6-dioxopiperidin-3-yl)phenyl]amino}heptanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(9-{[4-(2,6-dioxopiperidin-3-yl)phenyl]amino}nonanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-({4-[7-({3-[(2,6-dioxopiperidin-3-yl)amino]phenyl}amino)heptanoyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[9-({3-[(2,6-dioxopiperidin-3-yl)amino]phenyl}amino) nonanoyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-{[4-(9-{[3-(2,4-dioxo-1,3-diazinan-1-yl)-1-methyl-1H-indazol-6-yl]amino}nonanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(9-{[3-(2,6-dioxopiperidin-3-yl)-1-methyl-1H-indazol-6-yl]amino}nonanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-({4-[5-(2-{[2-(2,6-dioxopiperidin-3-yl)-1-oxo-2,3-dihydro-1H-isoindol-4-yl]oxy}acetamido)pentanoyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[7-(2-{[2-(2,6-dioxopiperidin-3-yl)-1-oxo-2,3-dihydro-1H-isoindol-4-yl]oxy}acetamido)heptanoyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[11-(2-{[2-(2,6-dioxopiperidin-3-yl)-1-oxo-2,3-dihydro-1H-isoindol-4-yl]oxy}acetamido)undecanoyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[13-(2-{[2-(2,6-dioxopiperidin-3-yl)-1-oxo-2,3-dihydro-1H-isoindol-4-yl]oxy}acetamido)tridecanoyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[15-(2-{[2-(2,6-dioxopiperidin-3-yl)-1-oxo-2,3-dihydro-1H-isoindol-4-yl]oxy}acetamido)pentadecanoyl]piperazin-1-yl}methyl)piperidin-4-yl]-2,5-difluorobenzamide;
N-[1-[5-(3-cyano-6-ethoxy-pyrazolo[1,5-a]pyridin-4-yl)-2-pyridyl]-4-[[2-[4-[4-[2-(2,6-dioxo-3-piperidyl)-1-oxo-isoindolin-5-yl]piperazin-1-yl]butylamino]-2-oxo-ethoxy]methyl]-4-piperidyl]-2,5-difluoro-benzamide;
N-[1-[5-(3-cyano-6-ethoxy-pyrazolo[1,5-a]pyridin-4-yl)-2-pyridyl]-4-[2-[4-[3-[4-[2-(2,6-dioxo-3-piperidyl)-3-oxo-isoindolin-5-yl]-1-piperidyl]propanoyl]piperazin-1-yl]ethoxymethyl]-4-piperidyl]-2,5-difluoro-benzamide;
N-[1-[5-(3-cyano-6-ethoxy-pyrazolo[1,5-a]pyridin-4-yl)-2-pyridyl]-4-[2-[4-[3-[4-[2-(2,6-dioxo-3-piperidyl)-3-oxo-isoindolin-5-yl]piperazin-1-yl]propanoyl]piperazin-1-yl]ethoxymethyl]-4-piperidyl]-2,5-difluoro-benzamide;
N-[1-[5-(3-cyano-6-ethoxy-pyrazolo[1,5-a]pyridin-4-yl)-2-pyridyl]-4-[2-[4-[3-[4-[2-(2,6-dioxo-3-piperidyl)-1-oxo-isoindolin-5-yl]piperazin-1-yl]propanoyl]piperazin-1-yl]ethoxymethyl]-4-piperidyl]-2,5-difluoro-benzamide;
N-[1-[5-(3-cyano-6-ethoxy-pyrazolo[1,5-a]pyridin-4-yl)-2-pyridyl]-4-[2-[4-[5-[4-[2-(2,6-dioxo-3-piperidyl)-1-oxo-isoindolin-5-yl]piperazin-1-yl]pentanoyl]piperazin-1-yl]ethoxymethyl]-4-piperidyl]-2,5-difluoro-benzamide;
N-[1-[5-(3-cyano-6-ethoxy-pyrazolo[1,5-a]pyridin-4-yl)-2-pyridyl]-4-[[2-[4-[[4-[2-(2,6-dioxo-3-piperidyl)-1-oxo-isoindolin-5-yl]piperazin-1-yl]methyl]-1-piperidyl]-2-oxo-ethoxy]methyl]-4-piperidyl]-2,5-difluoro-benzamide;
N-[1-[5-(3-cyano-6-ethoxy-pyrazolo[1,5-a]pyridin-4-yl)-2-pyridyl]-4-[2-[4-[3-[4-[2-(2,6-dioxo-3-piperidyl)-3-oxo-isoindolin-5-yl]-1-piperidyl]propyl]piperazin-1-yl]ethoxymethyl]-4-piperidyl]-2,5-difluoro-benzamide;
N-[1-[5-(3-cyano-6-ethoxy-pyrazolo[1,5-a]pyridin-4-yl)-2-pyridyl]-4-[[2-[6-[[4-[2-(2,6-dioxo-3-piperidyl)-1-oxo-isoindolin-5-yl]piperazin-1-yl]hexylamino]-2-oxo-ethoxy]methyl]-4-piperidyl]-2,5-difluoro-benzamide;
N-(1-(5-(3-cyano-6-ethoxypyrazolo[1,5-a]pyridin-4-yl)pyridin-2-yl)-4-((4-(3-(4-(3-(2,4-dioxotetrahydropyrimidin-1(2H)-yl)-1-methyl-1H-indazol-6-yl)piperazin-1-yl)propanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(5-(4-(3-(2,4-dioxotetrahydropyrimidin-1(2H)-yl)-1-methyl-1H-indazol-6-yl)piperazin-1-yl)pentanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(7-(4-(3-(2,4-dioxotetrahydropyrimidin-1(2H)-yl)-1-methyl-1H-indazol-6-yl)piperazin-1-yl)heptanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(6-(4-(3-(2,4-dioxotetrahydropyrimidin-1(2H)-yl)-1-methyl-1H-indazol-6-yl)piperidin-1-yl)hexanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(4-(4-(3-(2,4-dioxotetrahydropyrimidin-1(2H)-yl)-1-methyl-1H-indazol-6-yl)piperazin-1-yl)butanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(2-(4-(3-(2,4-dioxotetrahydropyrimidin-1(2H)-yl)-1-methyl-1H-indazol-6-yl)piperidin-1-yl)acetyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(3-(4-(3-(2,4-dioxotetrahydropyrimidin-1(2H)-yl)-1-methyl-1H-indazol-6-yl)piperidin-1-yl)propanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(4-(4-(3-(2,4-dioxotetrahydropyrimidin-1(2H)-yl)-1-methyl-1H-indazol-6-yl)piperidin-1-yl)butanoyl)piperazin-1-yl)methyl)piperidin-4-yl)-2,5-difluorobenzamide;
N-[1-[5-(3-cyano-6-ethoxy-pyrazolo[1,5-a]pyridin-4-yl)-2-pyridyl]-4-[[1-[6-[4-[2-(2,6-dioxo-3-piperidyl)-1-oxo-isoindolin-5-yl]piperazin-1-yl]hexyl]triazol-4-yl]methoxymethyl]-4-piperidyl]-2,5-difluoro-benzamide;
N-[1-[5-(3-cyano-6-ethoxy-pyrazolo[1,5-a]pyridin-4-yl)-2-pyridyl]-4-[[1-[4-[4-[2-(2,6-dioxo-3-piperidyl)-1-oxo-isoindolin-5-yl]piperazin-1-yl]butyl]triazol-4-yl]methoxymethyl]-4-piperidyl]-2,5-difluoro-benzamide;
N-[1-[5-(3-cyano-6-ethoxy-pyrazolo[1,5-a]pyridin-4-yl)-2-pyridyl]-4-[[1-[2-[4-[2-(2,6-dioxo-3-piperidyl)-1-oxo-isoindolin-5-yl]piperazin-1-yl]ethyl]triazol-4-yl]methoxymethyl]-4-piperidyl]-2,5-difluoro-benzamide;
N-(1-(5-(3-cyano-6-ethoxypyrazolo[1,5-a]pyridin-4-yl)pyridin-2-yl)-4-(((1-(3-(4-(2-(2,6-dioxopiperidin-3-yl)-1-oxoisoindolin-5-yl)piperazin-1-yl) propyl)-1H-1,2,3-triazol-4-yl) methoxy)methyl)piperidin-4-yl)-2,5-difluorobenzamide;
N-[4-benzyl-1-[5-(3-cyano-6-ethoxy-pyrazolo[1,5-a]pyridin-4-yl)-2-pyridyl]-4-piperidyl]-4-[3-[4-[2-(2,6-dioxo-3-piperidyl)-3-oxo-isoindolin-5-yl]-1-piperidyl]propanoyl]piperazine-1-carboxamide;
N-[4-benzyl-1-[5-(3-cyano-6-ethoxy-pyrazolo[1,5-a]pyridin-4-yl)-2-pyridyl]-4-piperidyl]-1-[3-[4-[2-(2,6-dioxo-3-piperidyl)-3-oxo-isoindolin-5-yl]-1-piperidyl]propanoyl]piperidine-4-carboxamide;
N-[4-benzyl-1-[5-(3-cyano-6-ethoxy-pyrazolo[1,5-a]pyridin-4-yl)-2-pyridyl]-4-piperidyl]-2-[4-[3-[4-[2-(2,6-dioxo-3-piperidyl)-3-oxo-isoindolin-5-yl]-1-piperidyl]propanoyl]piperazin-1-yl]acetamide;
N-[4-benzyl-1-[5-(3-cyano-6-ethoxy-pyrazolo[1,5-a]pyridin-4-yl)-2-pyridyl]-4-piperidyl]-2-[1-[3-[4-[2-(2,6-dioxo-3-piperidyl)-3-oxo-isoindolin-5-yl]-1-piperidyl]propanoyl]-4-piperidyl]acetamide;
N-[4-benzyl-1-[5-(3-cyano-6-ethoxy-pyrazolo[1,5-apyridin-4-yl)-2-pyridyl]-4-piperidyl]-4-[[4-[2-(2,6-dioxo-3-piperidyl)-3-oxo-isoindolin-5-yl]-1-piperidyl]methyl]piperidine-1-carboxamide;
N-[4-benzyl-1-[5-(3-cyano-6-ethoxy-pyrazolo[1,5-a]pyridin-4-yl)-2-pyridyl]-4-piperidyl]-4-[4-[2-(2,6-dioxo-3-piperidyl)-3-oxo-isoindolin-5-yl]-1-piperidyl]piperidine-1-carboxamide;
N-[4-benzyl-1-[5-(3-cyano-6-ethoxy-pyrazolo[1,5-a]pyridin-4-yl)-2-pyridyl]-4-piperidyl]-2-[4-[4-[2-(2,6-dioxo-3-piperidyl)-3-oxo-isoindolin-5-yl]-1-piperidyl]methyl]-1-piperidyl]acetamide;
N-(1-(5-(3-cyano-6-ethoxypyrazolo[1,5-a]pyridin-4-yl)pyridin-2-yl)-4-((4-(3-(4-(4-((2,6-dioxopiperidin-3-yl)amino)phenyl)piperazin-1-yl)propanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(3-(4-(3-((2,6-dioxopiperidin-3-yl)amino)phenyl)piperazin-1-yl)propanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(3-(4-(4-(2,6-dioxopiperidin-3-yl)phenyl)piperazin-1-yl)propanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(3-(4-(3-(2,6-dioxopiperidin-3-yl)phenyl)piperazin-1-yl)propanoyl)piperazin-1-yl)methyl)piperidin-4-yl)-2,5-difluorobenzamide;
N-[1-(5-{3-cyano-6-ethoxypyrazolo[1,5-a]pyridin-4-yl}pyridin-2-yl)-4-{[4-(1-{2-[2-(2,6-dioxopiperidin-3-yl)-1-oxo-2,3-dihydro-1H-isoindol-5-yl]acetyl}azetidin-3-yl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-[(4-{1-[2-(2,6-dioxopiperidin-3-yl)-1-oxo-2,3-dihydro-1H-isoindole-5-carbonyl]azetidin-3-yl}piperazin-1-yl)methyl]piperidin-4-yl]-2,5-difluorobenzamide;
N-[1-(5-{3-cyano-6-ethoxypyrazolo[1,5-a]pyridin-4-yl}pyridin-2-yl)-4-[(4-{1-[3-(2,4-dioxo-1,3-diazinan-1-yl)-1-methyl-1H-indazole-6-carbonyl]azetidin-3-yl}piperazin-1-yl)methyl]piperidin-4-yl]-2,5-difluorobenzamide;
N-[1-(5-{3-cyano-6-ethoxypyrazolo[1,5-a]pyridin-4-yl}pyridin-2-yl)-4-({4-[1-(2-{[2-(2,6-dioxopiperidin-3-yl)-1-oxo-2,3-dihydro-1H-isoindol-5-yl]oxy}acetyl)azetidin-3-yl]piperazin-1-yl}methyl)piperidin-4-yl]-2,5-difluorobenzamide;
N-[4-benzyl-1-(5-{3-cyano-6-ethoxypyrazolo[1,5-a]pyridin-4-yl}pyridin-2-yl)piperidin-4-yl]-1-(3-{4-[2-(2,6-dioxopiperidin-3-yl)-1-oxo-2,3-dihydro-1H-isoindol-5-yl]piperidin-1-yl}propanoyl)piperidine-4-carboxamide;
N-(1-(5-(3-cyano-6-ethoxypyrazolo[1,5-a]pyridin-4-yl)pyridin-2-yl)-4-((4-((4-(2-(2,6-dioxopiperidin-3-yl)-3-oxoisoindolin-5-yl)piperazin-1-yl)methyl)piperidin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(4-(2-(2,6-dioxopiperidin-3-yl)-3-oxoisoindolin-5-yl)piperazin-1-yl)piperidin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-((4-(2-(2,6-dioxopiperidin-3-yl)-1-oxoisoindolin-5-yl)piperazin-1-yl)methyl)piperidin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)azetidine-1-carbonyl)piperidin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)azetidin-1-yl)piperidin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-((3-(4-(2-(2,6-dioxopiperidin-3-yl)-3-oxoisoindolin-5-yl)piperidin-1-yl)azetidin-1-yl)methyl)piperidin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)propanoyl)piperazin-1-yl)methyl)piperidin-4-yl)-1-fluorocyclopropane-1-carboxamide;
N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)cyclobutane-1-carbonyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(1-(1-(2-(2,6-dioxopiperidin-3-yl)-3-oxoisoindolin-5-yl)piperidin-4-yl)azetidin-3-yl)piperazin-1-yl)methyl)piperidin-4-yl)-2,5-difluorobenzamide;
1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)propanoyl)piperazin-1-yl)methyl)-N-isopropylpiperidine-4-carboxamide;
1-(5-(3-Cyano-6-ethoxypyrazolo[1,5-a]pyridin-4-yl)pyridin-2-yl)-4-((4-(3-(4-(2-(2,6-dioxopiperidin-3-yl)-1-oxoisoindolin-5-yl)piperazin-1-yl)propanoyl)piperazin-1-yl)methyl)-N-isopropylpiperidine-4-carboxamide;
1-(5-{3-cyano-6-ethoxypyrazolo[1,5-a]pyridin-4-yl}pyridin-2-yl)-4-{[4-(3-{4-[2-(2,6-dioxopiperidin-3-yl)-3-oxo-2,3-dihydro-1H-isoindol-5-yl]piperidin-1-yl}propanoyl)piperazin-1-yl]methyl}-N-(2-methylpropyl)piperidine-4-carboxamide;
N-(1-(5-(3-cyano-6-ethoxypyrazolo[1,5-a]pyridin-4-yl)pyridin-2-yl)-4-((4-(1-(3-(2,4-dioxotetrahydropyrimidin-1(2H)-yl)-1-methyl-1H-indazol-6-yl)piperidine-4-carbonyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(3-(4-(2-(3-methyl-2,6-dioxopiperidin-3-yl)-3-oxoisoindolin-5-yl)piperidin-1-yl)propanoyl)piperazin-1-yl)methyl)piperidin-4-yl)-2,5-difluorobenzamide;
(S)—N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)propanoyl)piperazin-1-yl)methyl)piperidin-4-yl)-2,5-difluorobenzamide;
(R)—N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)propanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(6-(4-(2-(2,6-dioxopiperidin-3-yl)-1-oxoisoindolin-5-yl)piperazin-1-yl)spiro[3.3]heptane-2-carbonyl)piperazin-1-yl)methyl)piperidin-4-yl)-2,5-difluorobenzamide;
6-(4-{[1-(5-{3-cyano-6-ethoxypyrazolo[1,5-a]pyridin-4-yl}pyridin-2-yl)-4-(2,5-difluorobenzamido)piperidin-4-yl]methyl}piperazine-1-carbonyl)-N-[3-(2,4-dioxo-1,3-diazinan-1-yl)-1-methyl-1H-indazol-6-yl]spiro[3.3]heptane-2-carboxamide;
N-[1-(5-{3-cyano-6-ethoxypyrazolo[1,5-a]pyridin-4-yl}pyridin-2-yl)-4-{[4-(6-{4-[2-(2,6-dioxopiperidin-3-yl)-1-oxo-2,3-dihydro-1H-isoindol-5-yl]piperazine-1-carbonyl}spiro[3.3]heptane-2-carbonyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(6-{4-[2-(2,6-dioxopiperidin-3-yl)-3-oxo-2,3-dihydro-1H-isoindol-5-yl]piperazine-1-carbonyl}spiro[3.3]heptane-2-carbonyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[9-(3-{4-[2-(2,6-dioxopiperidin-3-yl)-1-oxo-2,3-dihydro-1H-isoindol-5-yl]piperazin-1-yl}propanoyl)-3,9-diazaspiro[5.5]undecan-3-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[9-(3-{4-[2-(2,6-dioxopiperidin-3-yl)-1-oxo-2,3-dihydro-1H-isoindol-5-yl]piperidin-1-yl}propanoyl)-3,9-diazaspiro[5.5]undecan-3-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[6-(3-{4-[2-(2,6-dioxopiperidin-3-yl)-1-oxo-2,3-dihydro-1H-isoindol-5-yl]piperidin-1-yl}propanoyl)-2,6-diazaspiro[3.3]heptan-2-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(3-{9-[2-(2,6-dioxopiperidin-3-yl)-1-oxo-2,3-dihydro-1H-isoindol-5-yl]-3,9-diazaspiro[5.5]undecan-3-yl}-3-oxopropyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(3-{9-[2-(2,6-dioxopiperidin-3-yl)-1-oxo-2,3-dihydro-1H-isoindol-5-yl]-3,9-diazaspiro[5.5]undecan-3-yl}propanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(6-{4-[2-(2,6-dioxopiperidin-3-yl)-1-oxo-2,3-dihydro-1H-isoindol-5-yl]piperidin-1-yl}spiro[3.3]heptane-2-carbonyl)piperazin-1-yl]methyl}piperidin-4-yl]-2,5-difluorobenzamide
N-[1-(5-{3-cyano-6-ethoxypyrazolo[1,5-a]pyridin-4-yl}pyridin-2-yl)-4-{[4-(6-{[3-(2,4-dioxo-1,3-diazinan-1-yl)-1-methyl-1H-indazol-6-yl]amino}spiro[3.3]heptane-2-carbonyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(6-{4-[2-(2,6-dioxopiperidin-3-yl)-1-oxo-2,3-dihydro-1H-isoindol-5-yl]piperazine-1-carbonyl}spiro[3.3]heptan-2-yl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(6-{4-[2-(2,6-dioxopiperidin-3-yl)-1-oxo-2,3-dihydro-1H-isoindol-5-yl]piperidine-1-carbonyl}spiro[3.3]heptan-2-yl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-({9-[2-(2,6-dioxopiperidin-3-yl)-3-oxo-2,3-dihydro-1H-isoindole-5-carbonyl]-3,9-diazaspiro[5.5]undecan-3-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-{[6-(2-{[2-(2,6-dioxopiperidin-3-yl)-1-oxo-2,3-dihydro-1H-isoindol-5-yl]oxy}acetyl)-2,6-diazaspiro[3.3]heptan-2-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-[(9-{2-[2-(2,6-dioxopiperidin-3-yl)-1-oxo-2,3-dihydro-1H-isoindol-5-yl]acetyl}-3,9-diazaspiro[5.5]undecan-3-yl)methyl]piperidin-4-yl]-2,5-difluorobenzamide;
N-[1-(5-{3-cyano-6-ethoxypyrazolo[1,5-a]pyridin-4-yl}pyridin-2-yl)-4-{[9-(2-{[2-(2,6-dioxopiperidin-3-yl)-1-oxo-2,3-dihydro-1H-isoindol-5-yl]oxy}acetyl)-3,9-diazaspiro[5.5]undecan-3-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-({4-[2-(3-{4-[2-(2,6-dioxopiperidin-3-yl)-1-oxo-2,3-dihydro-1H-isoindol-5-yl]piperazin-1-yl}-3-oxopropoxy)ethyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[2-(3-{4-[2-(2,6-dioxopiperidin-3-yl)-3-oxo-2,3-dihydro-1H-isoindol-5-yl]piperidin-1-yl}-3-oxopropoxy)ethyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[2-(2-{[3-(2,4-dioxo-1,3-diazinan-1-yl)-1-methyl-1H-indazol-6-yl]carbamoyl}ethoxy)ethyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[2-(3-{4-[2-(2,6-dioxopiperidin-3-yl)-3-oxo-2,3-dihydro-1H-isoindol-5-yl]piperazin-1-yl}-3-oxopropoxy)ethyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[3-(2-{4-[2-(2,6-dioxopiperidin-3-yl)-1-oxo-2,3-dihydro-1H-isoindol-5-yl]piperazin-1-yl}ethoxy)propanoyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[3-(2-{[3-(2,4-dioxo-1,3-diazinan-1-yl)-1-methyl-1H-indazol-6-yl]amino}ethoxy)propanoyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-[(4-{2-[2-(2,6dioxopiperidin-3-yl)-3-oxo-2,3-dihydro-1H-isoindol-5-yl]acetyl}piperazin-1-yl)methyl]piperidin-4-yl]-2,5-difluorobenzamide;
N-[1-(5-{3-cyano-6-ethoxypyrazolo[1,5-a]pyridin-4-yl}pyridin-2-yl)-4-{[4-(2-{[2-(2,6-dioxopiperidin-3-yl)-1-oxo-2,3-dihydro-1H-isoindol-5-yl]oxy}acetyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-[(4-{2-[2-(2-{[3-(2,4-dioxo-1,3-diazinan-1-yl)-1-methyl-1H-indazol-6-yl]carbamoyl}ethoxy)ethoxy]ethyl}piperazin-1-yl)methyl]piperidin-4-yl]-2,5-difluorobenzamide;
N-[1-(5-{3-cyano-6-ethoxypyrazolo[1,5-a]pyridin-4-yl}pyridin-2-yl)-4-[(4-{2-[2-(3-{4-[2-(2,6-dioxopiperidin-3-yl)-1-oxo-2,3-dihydro-1H-isoindol-5-yl]piperazin-1-yl}-3-oxopropoxy)ethoxy]ethyl}piperazin-1-yl)methyl]piperidin-4-yl]-2,5-difluorobenzamide;
N-[1-(5-{3-cyano-6-ethoxypyrazolo[1,5-a]pyridin-4-yl}pyridin-2-yl)-4-{[4-({1-[2-(2,6-dioxopiperidin-3-yl)-3-oxo-2,3-dihydro-1H-isoindole-5-carbonyl]piperidin-4-yl}methyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-({4-[(1-{2-[2-(2,6-dioxopiperidin-3-yl)-1-oxo-2,3-dihydro-1H-isoindol-4-yl]acetyl}piperidin-4-yl)methyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[(1-{2-[2-(2,6-dioxopiperidin-3-yl)-1-oxo-2,3-dihydro-1H-isoindol-5-yl]acetyl}piperidin-4-yl)methyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[(1-{2-[2-(2,6-dioxopiperidin-3-yl)-3-oxo-2,3-dihydro-1H-isoindol-5-yl]acetyl}piperidin-4-yl)methyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-[(4-{[1-(2-{[2-(2,6-dioxopiperidin-3-yl)-1-oxo-2,3-dihydro-1H-isoindol-5-yl]oxy}acetyl)piperidin-4-yl]methyl}piperazin-1-yl)methyl]piperidin-4-yl]-2,5-difluorobenzamide;
N-[1-(5-{3-cyano-6-ethoxypyrazolo[1,5-a]pyridin-4-yl}pyridin-2-yl)-4-{[4-(3-{4-[2-(2,6-dioxopiperidin-3-yl)-3-oxo-2,3-dihydro-1H-isoindol-5-yl]piperazin-1-yl}-3-oxopropyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(3-{4-[2-(2,6-dioxopiperidin-3-yl)-3-oxo-2,3-dihydro-1H-isoindol-5-yl]piperidin-1-yl}-3-oxopropyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(3-{4-[2-(2,6-dioxopiperidin-3-yl)-1,3-dioxo-2,3-dihydro-1H-isoindol-5-yl]piperidin-1-yl}-3-oxopropyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-({4-[3-(4-{[2-(2,6-dioxopiperidin-3-yl)-1-oxo-2,3-dihydro-1H-isoindol-5-yl]oxy}piperidin-1-yl)propanoyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-{[4-(3-{3-[2-(2,6-dioxopiperidin-3-yl)-1-oxo-2,3-dihydro-1H-isoindol-5-yl]azetidin-1-yl}propanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-({4-[3-(3-{[2-(2,6-dioxopiperidin-3-yl)-1-oxo-2,3-dihydro-1H-isoindol-5-yl]oxy}azetidin-1-yl)propanoyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-{[9-(2-{4-[2-(2,6-dioxopiperidin-3-yl)-3-oxo-2,3-dihydro-1H-isoindol-5-yl]piperidin-1-yl}-2-oxoethyl)-3-azaspiro[5.5]undecan-3-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[9-(2-{4-[2-(2,6-dioxopiperidin-3-yl)-1-oxo-2,3-dihydro-1H-isoindol-5-yl]piperidin-1-yl}-2-oxoethyl)-3-azaspiro[5.5]undecan-3-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[9-(2-{4-[2-(2,6-dioxopiperidin-3-yl)-3-oxo-2,3-dihydro-1H-isoindol-5-yl]piperazin-1-yl}-2-oxoethyl)-3-azaspiro[5.5]undecan-3-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[9-(2-{4-[2-(2,6-dioxopiperidin-3-yl)-1-oxo-2,3-dihydro-1H-isoindol-5-yl]piperazin-1-yl}-2-oxoethyl)-3-azaspiro[5.5]undecan-3-yl]methyl}piperidin-4-yl]-2,5-difluorobenzamide;
4-[6-(4-{[4-(7-{4-[2-(2,6-dioxopiperidin-3-yl)-1-oxo-2,3-dihydro-1H-isoindol-5-yl]piperazin-1-yl}heptanoyl)piperazin-1-yl]methyl}piperidin-1-yl)pyridin-3-yl]-6-ethoxypyrazolo[1,5-a]pyridine-3-carbonitrile;
4-[6-(4-{[4-(3-{4-[2-(2,6-dioxopiperidin-3-yl)-3-oxo-2,3-dihydro-1H-isoindol-5-yl]piperidin-1-yl}propanoyl)piperazin-1-yl]methyl}piperidin-1-yl)pyridin-3-yl]-6-ethoxypyrazolo[1,5-a]pyridine-3-carbonitrile;
N-[1-(5-{3-cyano-6-ethoxypyrazolo[1,5-a]pyridin-4-yl}pyridin-2-yl)-4-[(methylamino)methyl]piperidin-4-yl]-2,5-difluorobenzamide;
4-[6-(4-{[4-(7-{4-[2-(2,6-dioxopiperidin-3-yl)-1-oxo-2,3-dihydro-1H-isoindol-5-yl]piperazin-1-yl}heptanoyl)piperazin-1-yl]methyl}-4-methylpiperidin-1-yl)pyridin-3-yl]-6-ethoxypyrazolo[1,5-a]pyridine-3-carbonitrile;
4-[6-(4-{[4-(3-{4-[2-(2,6-dioxopiperidin-3-yl)-1-oxo-2,3-dihydro-1H-isoindol-5-yl]piperidin-1-yl}propanoyl)piperazin-1-yl]methyl}-4-methylpiperidin-1-yl)pyridin-3-yl]-6-ethoxypyrazolo[1,5-a]pyridine-3-carbonitrile;
4-[6-(4-{[4-(7-{4-[2-(2,6-dioxopiperidin-3-yl)-1-oxo-2,3-dihydro-1H-isoindol-5-yl]piperazin-1-yl}heptanoyl)piperazin-1-yl]methyl}-4-ethylpiperidin-1-yl)pyridin-3-yl]-6-ethoxypyrazolo[1,5-a]pyridine-3-carbonitrile;
4-[6-(4-{[4-(3-{4-[2-(2,6-dioxopiperidin-3-yl)-1-oxo-2,3-dihydro-1H-isoindol-5-yl]piperidin-1-yl}propanoyl)piperazin-1-yl]methyl}-4-ethylpiperidin-1-yl)pyridin-3-yl]-6-ethoxypyrazolo[1,5-a]pyridine-3-carbonitrile;
4-[6-(4-{[4-(3-{4-[2-(2,6-dioxopiperidin-3-yl)-3-oxo-2,3-dihydro-1H-isoindol-5-yl]piperidin-1-yl}propanoyl)piperazin-1-yl]methyl}-4-ethylpiperidin-1-yl)pyridin-3-yl]-6-ethoxypyrazolo[1,5-a]pyridine-3-carbonitrile;
4-[6-(4-{[4-(2-{[2-(2,6-dioxopiperidin-3-yl)-1-oxo-2,3-dihydro-1H-isoindol-5-yl]oxy}acetyl)piperazin-1-yl]methyl}-4-ethylpiperidin-1-yl)pyridin-3-yl]-6-ethoxypyrazolo[1,5-a]pyridine-3-carbonitrile;
N-(1-(5-(3-cyano-6-ethoxypyrazolo[1,5-a]pyridin-4-yl)pyridin-2-yl)-4-(9-(2-(2,6-dioxopiperidin-3-yl)-1-oxoisoindolin-5-yl)-3,9-diazaspiro[5.5]undecane-3-carbonyl)piperidin-4-yl)-2,5-difluorobenzamide;
N-[1-(5-{3-cyano-6-ethoxypyrazolo[1,5-a]pyridin-4-yl}pyridin-2-yl)-4-{[4-(7-{4-[2-(1-methyl-2,6-dioxopiperidin-3-yl)-1-oxo-2,3-dihydro-1H-isoindol-5-yl]piperazin-1-yl}heptanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-[(5-{4-[2-(2,6-dioxopiperidin-3-yl)-1-oxo-2,3-dihydro-1H-isoindol-5-yl]piperazin-1-yl}-N-methylpentanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(7-{4-[2-(2,6-dioxopiperidin-3-yl)-1-oxo-2,3-dihydro-1H-isoindol-5-yl]piperazin-1-yl}-N-methylheptanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(3-{4-[2-(2,6-dioxopiperidin-3-yl)-1-oxo-2,3-dihydro-1H-isoindol-5-yl]piperidin-1-yl}-N-methylpropanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(5-{4-[2-(2,6-dioxopiperidin-3-yl)-1-oxo-2,3-dihydro-1H-isoindol-5-yl]piperidin-1-yl}-N-methylpentanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(7-{4-[2-(2,6-dioxopiperidin-3-yl)-1-oxo-2,3-dihydro-1H-isoindol-5-yl]piperidin-1-yl}-N-methylheptanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(3-{4-[2-(2,6-dioxopiperidin-3-yl)-3-oxo-2,3-dihydro-1H-isoindol-5-yl]piperazin-1-yl}-N-methylpropanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(5-{4-[2-(2,6-dioxopiperidin-3-yl)-3-oxo-2,3-dihydro-1H-isoindol-5-yl]piperazin-1-yl}-N-methylpentanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(7-{4-[2-(2,6-dioxopiperidin-3-yl)-3-oxo-2,3-dihydro-1H-isoindol-5-yl]piperazin-1-yl}-N-methylheptanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(3-{4-[2-(2,6-dioxopiperidin-3-yl)-3-oxo-2,3-dihydro-1H-isoindol-5-yl]piperidin-1-yl}-N-methylpropanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(5-{4-[2-(2,6-dioxopiperidin-3-yl)-3-oxo-2,3-dihydro-1H-isoindol-5-yl]piperidin-1-yl}-N-methylpentanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(7-{4-[2-(2,6-dioxopiperidin-3-yl)-3-oxo-2,3-dihydro-1H-isoindol-5-yl]piperidin-1-yl}-N-methylheptanamido)methyl]piperidin-4-yl]-2,5-difluorobenzamide;
4-[6-(4-{[4-(3-{4-[2-(2,6-dioxopiperidin-3-yl)-3-oxo-2,3-dihydro-1H-isoindol-5-yl]piperidin-1-yl}propanoyl)piperazin-1-yl]methyl}-4-methylpiperidin-1-yl)pyridin-3-yl]-6-ethoxypyrazolo[1,5-a]pyridine-3-carbonitrile;
1-(5-{3-cyano-6-ethoxypyrazolo[1,5-a]pyridin-4-yl}pyridin-2-yl)-4-(2,5-difluorobenzamido)-N-(6-{[4-(2,4-dioxo-1,3-diazinan-1-yl)phenyl]amino}hexyl)piperidine-4-carboxamide;
1-(5-{3-cyano-6-ethoxypyrazolo[1,5-a]pyridin-4-yl}pyridin-2-yl)-4-(2,5-difluorobenzamido)-N-(6-{[3-(2,4-dioxo-1,3-diazinan-1-yl)-1-methyl-1H-indazol-7-yl]amino}hexyl)piperidine-4-carboxamide;
1-(5-{3-cyano-6-ethoxypyrazolo[1,5-a]pyridin-4-yl}pyridin-2-yl)-4-(2,5-difluorobenzamido)-N-(10-{[3-(2,4-dioxo-1,3-diazinan-1-yl)-1-methyl-1H-indazol-7-yl]amino}decyl)piperidine-4-carboxamide;
1-(5-{3-cyano-6-ethoxypyrazolo[1,5-a]pyridin-4-yl}pyridin-2-yl)-4-(2,5-difluorobenzamido)-N-(6-{[3-(2,4-dioxo-1,3-diazinan-1-yl)-1-methyl-1H-indazol-6-yl]amino}hexyl)piperidine-4-carboxamide;
1-(5-{3-cyano-6-ethoxypyrazolo[1,5-a]pyridin-4-yl}pyridin-2-yl)-4-(2,5-difluorobenzamido)-N-(8-{[3-(2,4-dioxo-1,3-diazinan-1-yl)-1-methyl-1H-indazol-6-yl]amino}octyl)piperidine-4-carboxamide;
1-(5-{3-cyano-6-ethoxypyrazolo[1,5-a]pyridin-4-yl}pyridin-2-yl)-4-(2,5-difluorobenzamido)-N-(10-{[3-(2,4-dioxo-1,3-diazinan-1-yl)-1-methyl-1H-indazol-6-yl]amino}decyl)piperidine-4-carboxamide;
1-(5-{3-cyano-6-ethoxypyrazolo[1,5-a]pyridin-4-yl}pyridin-2-yl)-4-(2,5-difluorobenzamido)-N-(4-{4-[2-(2,6-dioxopiperidin-3-yl)-1-oxo-2,3-dihydro-1H-isoindol-5-yl]piperazin-1-yl}butyl)piperidine-4-carboxamide;
1-(5-{3-cyano-6-ethoxypyrazolo[1,5-a]pyridin-4-yl}pyridin-2-yl)-4-(2,5-difluorobenzamido)-N-(6-{4-[2-(2,6-dioxopiperidin-3-yl)-1-oxo-2,3-dihydro-1H-isoindol-5-yl]piperazin-1-yl}hexyl)piperidine-4-carboxamide;
1-(5-{3-cyano-6-ethoxypyrazolo[1,5-a]pyridin-4-yl}pyridin-2-yl)-4-(2,5-difluorobenzamido)-N-(2-{4-[2-(2,6-dioxopiperidin-3-yl)-1-oxo-2,3-dihydro-1H-isoindol-5-yl]piperidin-1-yl}ethyl)piperidine-4-carboxamide;
1-(5-{3-cyano-6-ethoxypyrazolo[1,5-a]pyridin-4-yl}pyridin-2-yl)-4-(2,5-difluorobenzamido)-N-(6-{4-[2-(2,6-dioxopiperidin-3-yl)-1-oxo-2,3-dihydro-1H-isoindol-5-yl]piperidin-1-yl}hexyl)piperidine-4-carboxamide;
1-(5-{3-cyano-6-ethoxypyrazolo[1,5-a]pyridin-4-yl}pyridin-2-yl)-4-(2,5-difluorobenzamido)-N-(2-{4-[2-(2,6-dioxopiperidin-3-yl)-3-oxo-2,3-dihydro-1H-isoindol-5-yl]piperazin-1-yl}ethyl)piperidine-4-carboxamide;
1-(5-{3-cyano-6-ethoxypyrazolo[1,5-a]pyridin-4-yl}pyridin-2-yl)-4-(2,5-difluorobenzamido)-N-(4-{4-[2-(2,6-dioxopiperidin-3-yl)-3-oxo-2,3-dihydro-1H-isoindol-5-yl]piperazin-1-yl}butyl)piperidine-4-carboxamide;
1-(5-{3-cyano-6-ethoxypyrazolo[1,5-a]pyridin-4-yl}pyridin-2-yl)-4-(2,5-difluorobenzamido)-N-(6-{4-[2-(2,6-dioxopiperidin-3-yl)-3-oxo-2,3-dihydro-1H-isoindol-5-yl]piperazin-1-yl}hexyl)piperidine-4-carboxamide;
1-(5-{3-cyano-6-ethoxypyrazolo[1,5-a]pyridin-4-yl}pyridin-2-yl)-4-(2,5-difluorobenzamido)-N-(2-{4-[2-(2,6-dioxopiperidin-3-yl)-3-oxo-2,3-dihydro-1H-isoindol-5-yl]piperidin-1-yl}ethyl)piperidine-4-carboxamide;
1-(5-{3-cyano-6-ethoxypyrazolo[1,5-a]pyridin-4-yl}pyridin-2-yl)-4-(2,5-difluorobenzamido)-N-(4-{4-[2-(2,6-dioxopiperidin-3-yl)-3-oxo-2,3-dihydro-1H-isoindol-5-yl]piperidin-1-yl}butyl)piperidine-4-carboxamide;
1-(5-{3-cyano-6-ethoxypyrazolo[1,5-a]pyridin-4-yl}pyridin-2-yl)-4-(2,5-difluorobenzamido)-N-(6-{4-[2-(2,6-dioxopiperidin-3-yl)-3-oxo-2,3-dihydro-1H-isoindol-5-yl]piperidin-1-yl}hexyl)piperidine-4-carboxamide;
N-[1-(5-{3-cyano-6-ethoxypyrazolo[1,5-a]pyridin-4-yl}pyridin-2-yl)-4-[(5-{4-[2-(2,6-dioxopiperidin-3-yl)-1-oxo-2,3-dihydro-1H-isoindol-5-yl]piperazin-1-yl}-N-(propan-2-yl)pentanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(7-{4-[2-(2,6-dioxopiperidin-3-yl)-1-oxo-2,3-dihydro-1H-isoindol-5-yl]piperazin-1-yl}-N-(propan-2-yl)heptanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(7-{4-[2-(2,6-dioxopiperidin-3-yl)-1-oxo-2,3-dihydro-1H-isoindol-5-yl]piperidin-1-yl}-N-(propan-2-yl)heptanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(3-{4-[2-(2,6-dioxopiperidin-3-yl)-3-oxo-2,3-dihydro-1H-isoindol-5-yl]piperazin-1-yl}-N-(propan-2-yl)propanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(5-{4-[2-(2,6-dioxopiperidin-3-yl)-3-oxo-2,3-dihydro-1H-isoindol-5-yl]piperazin-1-yl}-N-(propan-2-yl)pentanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(7-{4-[2-(2,6-dioxopiperidin-3-yl)-3-oxo-2,3-dihydro-1H-isoindol-5-yl]piperazin-1-yl}-N-(propan-2-yl)heptanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(5-{4-[2-(2,6-dioxopiperidin-3-yl)-3-oxo-2,3-dihydro-1H-isoindol-5-yl]piperidin-1-yl}-N-(propan-2-yl)pentanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[9-(2-{4-[2-(2,6-dioxopiperidin-3-yl)-3-oxo-2,3-dihydro-1H-isoindol-5-yl]piperidin-1-yl}-2-oxoethyl)-3-azaspiro[5.5]undecane-3-carbonyl]piperidin-4-yl]-2,5-difluorobenzamide;
N-[1-(5-{3-cyano-6-ethoxypyrazolo[1,5-a]pyridin-4-yl}pyridin-2-yl)-4-[9-(2-{4-[2-(2,6-dioxopiperidin-3-yl)-1-oxo-2,3-dihydro-1H-isoindol-5-yl]piperidin-1-yl}-2-oxoethyl)-3-azaspiro[5.5]undecane-3-carbonyl]piperidin-4-yl]-2,5-difluorobenzamide;
N-[1-(5-{3-cyano-6-ethoxypyrazolo[1,5-a]pyridin-4-yl}pyridin-2-yl)-4-[9-(2-{4-[2-(2,6-dioxopiperidin-3-yl)-3-oxo-2,3-dihydro-1H-isoindol-5-yl]piperazin-1-yl}-2-oxoethyl)-3-azaspiro[5.5]undecane-3-carbonyl]piperidin-4-yl]-2,5-difluorobenzamide;
N-[1-(5-{3-cyano-6-ethoxypyrazolo[1,5-a]pyridin-4-yl}pyridin-2-yl)-4-[9-(2-{4-[2-(2,6-dioxopiperidin-3-yl)-1-oxo-2,3-dihydro-1H-isoindol-5-yl]piperazin-1-yl}-2-oxoethyl)-3-azaspiro[5.5]undecane-3-carbonyl]piperidin-4-yl]-2,5-difluorobenzamide;
(S)—N-(1-(5-(3-cyano-6-ethoxypyrazolo[1,5-a]pyridin-4-yl)pyridin-2-yl)-4-((4-(7-(4-(2-(2,6-dioxopiperidin-3-yl)-3-oxoisoindolin-5-yl)piperazin-1-yl)heptanoyl)piperazin-1-yl)methyl)piperidin-4-yl)-2,5-difluorobenzamide;
(R)—N-(1-(5-(3-cyano-6-ethoxypyrazolo[1,5-a]pyridin-4-yl)pyridin-2-yl)-4-((4-(7-(4-(2-(2,6-dioxopiperidin-3-yl)-3-oxoisoindolin-5-yl)piperazin-1-yl)heptanoyl)piperazin-1-yl)methyl)piperidin-4-yl)-2,5-difluorobenzamide; or
N-[1-(5-{3-cyano-6-ethoxypyrazolo[1,5-a]pyridin-4-yl}pyridin-2-yl)-4-[(7-{4-[2-(2,6-dioxopiperidin-3-yl)-3-oxo-2,3-dihydro-1H-isoindol-5-yl]piperidin-1-yl}-N-(propan-2-yl)heptanamido)methyl]piperidin-4-yl]-2,5-difluorobenzamide.

In other embodiments, suitable compounds of the disclosure include:
6-{[1-(3-{4-[2-(2,6-dioxopiperidin-3-yl)-1-oxo-2,3-dihydro-1H-isoindol-5-yl]piperazin-1-yl}propanoyl)piperidin-4-yl]methoxy}-4-{6-[(1R,5S)-6-[(6-methoxypyridin-3-yl)methyl]-3,6-diazabicyclo[3.1.1]heptan-3-yl]pyridin-3-yl}pyrazolo[1,5-a]pyridine-3-carbonitrile;
6-{[1-(7-{4-[2-(2,6-dioxopiperidin-3-yl)-1-oxo-2,3-dihydro-1H-isoindol-5-yl]piperazin-1-yl}heptanoyl)piperidin-4-yl]methoxy}-4-{6-[(1R,5S)-6-[(6-methoxypyridin-3-yl)methyl]-3,6-diazabicyclo[3.1.1]heptan-3-yl]pyridin-3-yl}pyrazolo[1,5-a]pyridine-3-carbonitrile;
6-{[1-(3-{4-[2-(2,6-dioxopiperidin-3-yl)-1-oxo-2,3-dihydro-1H-isoindol-5-yl]piperidin-1-yl}propanoyl)piperidin-4-yl]methoxy}-4-{6-[(1R,5S)-6-[(6-methoxypyridin-3-yl)methyl]-3,6-diazabicyclo[3.1.1]heptan-3-yl]pyridin-3-yl}pyrazolo[1,5-a]pyridine-3-carbonitrile;
6-{[1-(5-{4-[2-(2,6-dioxopiperidin-3-yl)-1-oxo-2,3-dihydro-1H-isoindol-5-yl]piperidin-1-yl}pentanoyl)piperidin-4-yl]methoxy}-4-{6-[(1R,5S)-6-[(6-methoxypyridin-3-yl)methyl]-3,6-diazabicyclo[3.1.1]heptan-3-yl]pyridin-3-yl}pyrazolo[1,5-a]pyridine-3-carbonitrile;
6-{[1-(3-{4-[2-(2,6-dioxopiperidin-3-yl)-3-oxo-2,3-dihydro-1H-isoindol-5-yl]piperazin-1-yl}propanoyl)piperidin-4-yl]methoxy}-4-{6-[(1R,5S)-6-[(6-methoxypyridin-3-yl)methyl]-3,6-diazabicyclo[3.1.1]heptan-3-yl]pyridin-3-yl}pyrazolo[1,5-a]pyridine-3-carbonitrile;
6-{[1-(7-{4-[2-(2,6-dioxopiperidin-3-yl)-3-oxo-2,3-dihydro-1H-isoindol-5-yl]piperazin-1-yl}heptanoyl)piperidin-4-yl]methoxy}-4-{6-[(1R,5S)-6-[(6-methoxypyridin-3-yl)methyl]-3,6-diazabicyclo[3.1.1]heptan-3-yl]pyridin-3-yl}pyrazolo[1,5-a]pyridine-3-carbonitrile;
6-{[1-(3-{4-[2-(2,6-dioxopiperidin-3-yl)-3-oxo-2,3-dihydro-1H-isoindol-5-yl]piperidin-1-yl}propanoyl)piperidin-4-yl]methoxy}-4-{6-[(1R,5S)-6-[(6-methoxypyridin-3-yl)methyl]-3,6-diazabicyclo[3.1.1]heptan-3-yl]pyridin-3-yl}pyrazolo[1,5-a]pyridine-3-carbonitrile; or
6-{[1-(7-{4-[2-(2,6-dioxopiperidin-3-yl)-3-oxo-2,3-dihydro-1H-isoindol-5-yl]piperidin-1-yl}heptanoyl)piperidin-4-yl]methoxy}-4-{6-[(1R,5S)-6-[(6-methoxypyridin-3-yl)methyl]-3,6-diazabicyclo[3.1.1]heptan-3-yl]pyridin-3-yl}pyrazolo[1,5-a]pyridine-3-carbonitrile.

In some embodiments, the compound is a pharmaceutically acceptable salt of any one of the compounds described herein.

In some aspects, the present disclosure provides a compound being an isotopic derivative (e.g., isotopically labeled compound) of any one of the compounds of the Formulae disclosed herein.

In some embodiments, the compound is an isotopic derivative of any one of the compounds described in Table 1 and prodrugs and pharmaceutically acceptable salts thereof.

In some embodiments, the compound is an isotopic derivative of any one of the compounds described in Table 1 and pharmaceutically acceptable salts thereof.

In some embodiments, the compound is an isotopic derivative of any one of prodrugs of the compounds described in Table 1 and pharmaceutically acceptable salts thereof.

In some embodiments, the compound is an isotopic derivative of any one of the compounds described in Table 1.

It is understood that the isotopic derivative can be prepared using any of a variety of art-recognised techniques. For example, the isotopic derivative can generally be prepared by carrying out the procedures disclosed in the Schemes and/or in the Examples described herein, by substituting an isotopically labeled reagent for a non-isotopically labeled reagent.

In some embodiments, the isotopic derivative is a deuterium labeled compound.

In some embodiments, the isotopic derivative is a deuterium labeled compound of any one of the compounds of the Formulae disclosed herein.

The term “isotopic derivative”, as used herein, refers to a derivative of a compound in which one or more atoms are isotopically enriched or labelled. For example, an isotopic derivative of a compound of Formula (I) is isotopically enriched with regard to, or labelled with, one or more isotopes as compared to the corresponding compound of Formula (I). In some embodiments, the isotopic derivative is enriched with regard to, or labelled with, one or more atoms selected from ²H, ¹³C, ¹⁴C, ¹⁵N, ¹⁸O, ²⁹Si, ³¹P, and ³⁴S. In some embodiments, the isotopic derivative is a deuterium labeled compound (i.e., being enriched with ²H with regard to one or more atoms thereof). In some embodiments, the compound is a ¹⁸F labeled compound. In some embodiments, the compound is a ¹²³I labeled compound, a ¹²⁴I labeled compound, a ¹²⁵I labeled compound, a ¹²⁹I labeled compound, a ¹³¹I labeled compound, a ¹³⁵I labeled compound, or any combination thereof. In some embodiments, the compound is a ³³S labeled compound, a ³⁴S labeled compound, a ³⁵S labeled compound, a ³⁶S labeled compound, or any combination thereof.

It is understood that the ¹⁸F, ¹²³I, ¹²⁴I, ¹²⁵I, ¹²⁹I, ¹³¹I, ¹³⁵I, ³²S, ³⁴S, ³⁵S, and/or ³⁶S labeled compound, can be prepared using any of a variety of art-recognised techniques. For example, the deuterium labeled compound can generally be prepared by carrying out the procedures disclosed in the Schemes and/or in the Examples described herein, by substituting a ¹⁸F, ¹²⁵I, ¹²⁴I, ¹²⁵I, ¹²⁹I, ¹³¹I, ¹³⁵I, ³²S, ³⁴S, ³⁵S, and/or ³⁶S labeled reagent for a non-isotope labeled reagent.

A compound of the invention or a pharmaceutically acceptable salt or solvate thereof that contains one or more of the aforementioned ¹⁸F, ¹²³I, ¹²⁴I, ¹²⁵I, ¹²⁹I, ¹³¹I, ¹³⁵I, ³²S, ³⁴S, ³⁵S, and ³⁶S atom(s) is within the scope of the invention. Further, substitution with isotope (e.g., ¹⁸F, ¹²³I, ¹²⁴I, ¹²⁵I, ¹²⁹I, ¹³¹I, ¹³⁵I, ³²S, ³⁴S, ³⁵S, and/or ³⁶S) may afford certain therapeutic advantages resulting from greater metabolic stability, e.g., increased in vivo half-life or reduced dosage requirements.

For the avoidance of doubt it is to be understood that, where in this specification a group is qualified by “described herein”, the said group encompasses the first occurring and broadest definition as well as each and all of the particular definitions for that group.

A suitable pharmaceutically acceptable salt of a compound of the disclosure is, for example, an acid-addition salt of a compound of the disclosure which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the disclosure which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, diethylamine, trimethylamine, piperidine, morpholine or tris-(2-hydroxyethyl)amine.

It will be understood that the compounds of any one of the Formulae disclosed herein and any pharmaceutically acceptable salts thereof, comprise stereoisomers, mixtures of stereoisomers, polymorphs of all isomeric forms of said compounds.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral center” refers to a carbon atom bonded to four nonidentical substituents.

As used herein, the term “chiral isomer” means a compound with at least one chiral centre. Compounds with more than one chiral centre may exist either as an individual diastereomer or as a mixture of diastereomers, termed “diastereomeric mixture.” When one chiral centre is present, a stereoisomer may be characterised by the absolute configuration (R or S) of that chiral centre. Absolute configuration refers to the arrangement in space of the substituents attached to the chiral centre. The substituents attached to the chiral centre under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413. Cahn and Ingold, J. Chem Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerisation is possible, a chemical equilibrium of the tautomers will be reached. The exact ratio of the tautomers depends on several factors, including temperature, solvent and pH. The concept of tautomers that are interconvertible by tautomerisations is called tautomerism. Of the various types of tautomerism, that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterised by the absolute configuration of its asymmetric centre and is described by the R- and S-sequencing rules of Cahn and Prelog, or by the manner in which the molecule rotates the plane of polarised light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centres, such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centres (E- and Z-isomers). It is to be understood that the present disclosure encompasses all optical, diastereoisomers and geometric isomers and mixtures thereof that possess inflammasome inhibitory activity.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted compound disclosed herein. Suitable cations include sodium ion, potassium ion, magnesium ion, calcium ion, and an ammonium cation such as tetramethylammonium ion or diethylamine ion. The substituted compounds disclosed herein also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate, and if the solvent is alcohol, the solvate formed is an alcoholate. Hydrates are formed by the combination of one or more molecules of water with one molecule of the substance in which the water retains its molecular state as H2O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As used herein, the term “derivative” refers to compounds that have a common core structure and are substituted with various groups as described herein.

It is also to be understood that certain compounds of any one of the Formulae disclosed herein may exist in solvated as well as unsolvated forms such as, for example, hydrated forms. A suitable pharmaceutically acceptable solvate is, for example, a hydrate such as hemi-hydrate, a mono-hydrate, a di-hydrate or a tri-hydrate. It is to be understood that the disclosure encompasses all such solvated forms that possess inflammasome inhibitory activity.

It is also to be understood that certain compounds of any one of the Formulae disclosed herein may exhibit polymorphism, and that the disclosure encompasses all such forms, or mixtures thereof, which possess inflammasome inhibitory activity. It is generally known that crystalline materials may be analysed using conventional techniques such as X-Ray Powder Diffraction analysis, Differential Scanning calorimetry, Thermal Gravimetric Analysis, Diffuse Reflectance Infrared Fourier Transform (DRIFT) spectroscopy, Near Infrared (NIR) spectroscopy, solution and/or solid state nuclear magnetic resonance spectroscopy. The water content of such crystalline materials may be determined by Karl Fischer analysis.

Compounds of any one of the Formulae disclosed herein may exist in a number of different tautomeric forms and references to compounds of Formula (I) include all such forms. For the avoidance of doubt, where a compound can exist in one of several tautomeric forms, and only one is specifically described or shown, all others are nevertheless embraced by Formula (I). Examples of tautomeric forms include keto-, enol-, and enolate-forms, as in, for example, the following tautomeric pairs keto/enol (illustrated below), imine/enamine, amide/imino alcohol, amidine/amidine, nitroso/oxime, thioketone/enethiol, and nitro/aci-nitro.

Figure U.S. Pat. No. 11,319,319—20220503—C00141

The compounds of any one of the Formulae disclosed herein may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached Examples of prodrugs include derivatives containing in vivo cleavable alkyl or acyl substitutents at the ester or amide group in any one of the Formulae disclosed herein.

Accordingly, the present disclosure includes those compounds of any one of the Formulae disclosed herein as defined hereinbefore when made available by organic synthesis and when made available within the human or animal body by way of cleavage of a prodrug thereof. Accordingly, the present disclosure includes those compounds of any one of the Formulae disclosed herein that are produced by organic synthetic means and also such compounds that are produced in the human or animal body by way of metabolism of a precursor compound, that is a compound of any one of the Formulae disclosed herein may be a synthetically-produced compound or a metabolically-produced compound.

A suitable pharmaceutically acceptable prodrug of a compound of any one of the Formulae disclosed herein is one that is based on reasonable medical judgment as being suitable for administration to the human or animal body without undesirable pharmacological activities and without undue toxicity. Various forms of prodrug have been described, for example in the following documents a) Methods in Enzymology, Vol 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N Kakeya, et al., Chem. Pharm Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a compound of any one of the Formulae disclosed herein that possesses a hydroxy group is, for example, an in vivo cleavable ester or ether thereof. An in vivo cleavable ester or ether of a compound of any one of the Formulae disclosed herein containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C1-C10, alkanoyl groups such as acetyl, benzoyl, phenylacetyl and substituted benzoyl and phenylacetyl groups, C1-C10 alkoxycarbonyl groups such as ethoxycarbonyl, N,N—(C1-C6 alkyl) 2carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C1-C4 alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable prodrug of a compound of any one of the Formulae disclosed herein that possesses a carboxy group is, for example, an in vivo cleavable amide thereof, for example an amide formed with an amine such as ammonia, a C1-4alkylamine such as methylamine, a (C1-(C4 alkyl)2-amine such as dimethylamine, N-ethyl-N-methylamine or diethylamine, a C1-C4 alkoxy-C2-C4 alkylamine such as 2-methoxyethylamine, a phenyl-C1-C4 alkylamine such as benzylamine and amino acids such as glycine or an ester thereof.

A suitable pharmaceutically acceptable prodrug of a compound of any one of the Formulae disclosed herein that possesses an amino group is, for example, an in vivo cleavable amide derivative thereof. Suitable pharmaceutically acceptable amides from an amino group include, for example an amide formed with C1-C10 alkanoyl groups such as an acetyl, benzoyl, phenylacetyl and substituted benzoyl and phenylacetyl groups Examples of ring substituents on the phenylacetyl and benzoyl groups include aminomethyl, N-alkylaminomethyl, N,N-dialkylaminomethyl, morpholinomethyl, piperazin-1-ylmethyl and 4-(C1-C4 alkyl)piperazin-1-ylmethyl.

The in vivo effects of a compound of any one of the Formulae disclosed herein may be exerted in part by one or more metabolites that are formed within the human or animal body after administration of a compound of any one of the Formulae disclosed herein. As stated hereinbefore, the in vivo effects of a compound of any one of the Formulae disclosed herein may also be exerted by way of metabolism of a precursor compound (a prodrug).

### Methods of Use

A compound described herein can be used in an effective amount to treat a patient, typically a human, in need thereof, who have a disorder mediated by RET which can be a wild-type RET or mutant RET as described generally herein. In certain embodiments a compound of the present invention degrades an additional protein, for example an aurora kinase or VEGFR2. In certain embodiments a compound of the present invention degrades RET and aurora A kinase (AURKA).

Another aspect of the present invention provides a compound as described herein, or an enantiomer, diastereomer, or stereoisomer thereof, or pharmaceutically acceptable salt, hydrate, or solvate thereof, or a pharmaceutical composition, for use in the manufacture of a medicament for treating or preventing cancer in a patient in need thereof; wherein there is a need of RET inhibition for the treatment or prevention of cancer.

In certain embodiments, the method comprises administering an effective amount of the active compound or its salt as described herein, optionally including a pharmaceutically acceptable excipient, carrier, or adjuvant (i.e., a pharmaceutically acceptable composition), or optionally in combination or alternation with another bioactive agent or combination of agents, to a patient in need thereof.

In certain embodiments, the present invention provides a method of treating any of the disorders described herein, in a patient in need thereof.

In other embodiments, the patient is administered an additional therapeutic agent. In other embodiments, the compound as described herein, and the additional therapeutic agent are administered simultaneously or sequentially.

In certain embodiments, the application provides a method of preventing any of the disorders described herein, in a patient in need thereof.

In certain embodiments, the patient is a human.

Another aspect of the present invention provides a method of treating or preventing a proliferative disease. The method comprises administering an effective amount of a pharmaceutical composition comprising a compound as described herein, or an enantiomer, diastereomer, or stereoisomer thereof, or pharmaceutically acceptable salt, hydrate, or solvate thereof and optionally a pharmaceutically acceptable carrier to a patient in need thereof.

In some embodiments, the disease is mediated by RET, for example, RET plays a role in the initiation or development of the disease.

In certain embodiments, the RET mediated disorder is a benign growth, metastasis, neoplasm, tumor, solid tumor, rhabdoid tumor, carcinoma, leukemia, cancer, abnormal cellular proliferation, an amyloid-based proteinopathy, a proteinopathy, fibrotic disorder, inflammation, arthritis, pulmonary disorders, or immune disorders.

In certain embodiments, the RET mediated disorder is a cancer that has metastasized, for example a cancer that has metastasized to the brain. In certain embodiments the RET mediated disorder is a cancer that has metastasized to the brain, lungs bone, liver, peritoneum, adrenal gland, skin, or muscle.

In certain embodiments a compound of the present invention penetrates the blood brain barrier and can be used for the treatment of a CNS involved cancer or a cancer that has metastasized to the brain.

In certain embodiments, the disease or disorder is cancer or a proliferation disease.

In certain embodiments, the RET mediated disorder is an abnormal cell proliferation, including, but not limited to, a tumor or cancer, or a myelo- or lymphoproliferative disorder such as B- or T-cell lymphomas, multiple myeloma, Waldenstrom's macroglobulinemia, Wiskott-Aldrich syndrome, or a post-transplant lymphoproliferative disorder.

In certain embodiments, the hematological cancer is acute myelogenous leukemia (AML), acute lymphoblastic leukemia (ALL), lymphoblastic T-cell leukemia, chronic myelogenous leukemia (CML), chronic lymphocytic leukemia (CLL), hairy-cell leukemia, chronic neutrophilic leukemia (CNL), acute lymphoblastic T-cell leukemia, acute monocytic leukemia, plasmacytoma, immunoblastic large cell leukemia, mantle cell leukemia, multiple myeloma, megakaryoblastic leukemia, acute megakaryocytic leukemia, promyelocytic leukemia, mixed lineage leukemia (MLL), erythroleukemia, malignant lymphoma, Hodgkins lymphoma, non-Hodgkins lymphoma, lymphoblastic T-cell lymphoma, Burkitt's lymphoma, follicular lymphoma, B cell acute lymphoblastic leukemia, diffuse large B cell lymphoma, Myc and B-Cell Leukemia (BCL) 2 and/or BCL6 rearrangements/overexpression [double- and triple-hit lymphoma], myelodysplastic/myeloproliferative neoplasm, mantle cell lymphoma including bortezomib resistant mantle cell lymphoma.

Solid tumors that can be treated with the compounds described herein include, but are not limited to lung cancers, including small cell lung cancer (SCLC) and non-small cell lung cancer (NSCLC), breast cancers including inflammatory breast cancer, ER-positive breast cancer including tamoxifen resistant ER-positive breast cancer, and triple negative breast cancer, colon cancers, midline carcinomas, liver cancers, renal cancers, prostate cancers including castrate resistant prostate cancer (CRPC), brain cancers including gliomas, glioblastomas, neuroblastoma, and medulloblastoma including MYC-amplified medulloblastoma, colorectal cancers, Wilm's tumor, Ewing's sarcoma, rhabdomyosarcomas, ependymomas, head and neck cancers, melanomas, squamous cell carcinomas, ovarian cancers, pancreatic cancers including pancreatic ductal adenocarcinomas (PDAC) and pancreatic neuroendocrine tumors (PanNET), osteosarcomas, giant cell tumors of bone, thyroid cancers, bladder cancers, urothelial cancers, vulval cancers, cervical cancers, endometrial cancers, mesotheliomas, esophageal cancers, salivary gland cancers, gastric cancers, nasopharangeal cancers, buccal cancers, cancers of the mouth, GIST (gastrointestinal stromal tumors), NUT-midline carcinomas, testicular cancers, squamous cell carcinomas, hepatocellular carcinomas (HCC), MYCN driven solid tumors, and NUT midline carcinomas (NMC).

In further embodiments, the disease or disorder is sarcoma of the bones, muscles, tendons, cartilage, nerves, fat, or blood vessels.

In further embodiments, the disease or disorder is soft tissue sarcoma, bone sarcoma, or osteosarcoma.

In further embodiments, the disease or disorder is angiosarcoma, fibrosarcoma, liposarcoma, leiomyosarcoma, Karposi's sarcoma, osteosarcoma, gastrointestinal stromal tumor, synovial sarcoma, Pleomorphic sarcoma, chondrosarcoma, Ewing's sarcoma, reticulum cell sarcoma, meningiosarcoma, botryoid sarcoma, rhabdomyosarcoma, or embryonal rhabdomyosarcoma.

In further embodiments, the disease or disorder is multiple myeloma.

In other embodiments, the disease or disorder is inflammation, arthritis, rheumatoid arthritis, spondyiarthropathies, gouty arthritis, osteoarthritis, juvenile arthritis, and other arthritic conditions, neuroinflammation, allergy, pain, neuropathic pain, fever, pulmonary disorders, lung inflammation, adult respiratory distress chronic pulmonary inflammatory disease, and chronic obstructive pulmonary disease (COPD), liver disease and nephritis, gastrointestinal conditions, inflammatory bowel disease, Crohn's disease, gastritis, irritable bowel syndrome, ulcerative colitis, ulcerative diseases, gastric ulcers, autoimmune disease, graft vs. host reaction and allograft rejections, cancer, leukemia, lymphoma, colorectal cancer, brain cancer, bone cancer, epithelial call-derived neoplasia (epithelial carcinoma), basal cell carcinoma, adenocarcinoma, gastrointestinal cancer, lip cancer, mouth cancer, esophageal cancer, small bowel cancer, stomach cancer, colon cancer, liver cancer, bladder cancer, pancreas cancer, ovarian cancer, cervical cancer, lung cancer, breast cancer, skin cancer, squamous cell and/or basal cell cancers, prostate cancer, renal cell carcinoma, and other known cancers that affect epithelial cells throughout the body, chronic myelogenous leukemia (CML), acute myeloid leukemia (AML) and acute promyelocytic leukemia (APL), angiogenesis including neoplasia, metastasis, central nervous system disorders, central nervous system disorders having an inflammatory or apoptotic component, peripheral neuropathy, or B-Cell Lymphoma.

In other embodiments, the pharmaceutical composition comprising the compound as described herein and the additional therapeutic agent are administered simultaneously or sequentially.

In other embodiments, the disease or disorder is cancer. In further embodiments, the cancer is lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas, myelomas, solid tumors, hematological cancers or solid cancers.

In some embodiments, said method is used to treat or prevent a condition selected from autoimmune diseases, inflammatory diseases, proliferative and hyperproliferative diseases, and immunologically-mediated diseases. In other embodiments, said condition is selected from a proliferative disorder.

In certain embodiments, the RET mediated disorder is an immune disorder, including but not limited to, autoimmune disorders such as Addison disease, Celiac disease, dermatomyositis, Graves disease, thyroiditis, multiple sclerosis, pernicious anemia, reactive arthritis, lupus, or type I diabetes.

One aspect of this application provides compounds that are useful for the treatment of diseases, disorders, and conditions characterized by excessive or abnormal cell proliferation. Such diseases include, but are not limited to, a proliferative or hyperproliferative disease. Examples of proliferative and hyperproliferative diseases include, without limitation, cancer. The term “cancer” includes, but is not limited to, the following cancers: breast; ovary; cervix; prostate; testis, genitourinary tract; esophagus; larynx, glioblastoma; neuroblastoma; stomach; skin, keratoacanthoma; lung, epidermoid carcinoma, large cell carcinoma, small cell carcinoma, lung adenocarcinoma; bone; colon; colorectal; adenoma; pancreas, adenocarcinoma; thyroid, follicular carcinoma, undifferentiated carcinoma, papillary carcinoma; seminoma; melanoma; sarcoma; bladder carcinoma; liver carcinoma and biliary passages; kidney carcinoma; myeloid disorders; lymphoid disorders, Hodgkin's, hairy cells; buccal cavity and pharynx (oral), lip, tongue, mouth, pharynx; small intestine; colonrectum, large intestine, rectum, brain and central nervous system; chronic myeloid leukemia (CML), and leukemia. The term “cancer” includes, but is not limited to, the following cancers: myeloma, lymphoma, or a cancer selected from gastric, renal, or and the following cancers: head and neck, oropharangeal, non-small cell lung cancer (NSCLC), endometrial, hepatocarcinoma, Non-Hodgkins lymphoma, and pulmonary.

The term “cancer” refers to any cancer caused by the proliferation of malignant neoplastic cells, such as tumors, neoplasms, carcinomas, sarcomas, leukemias, lymphomas and the like. For example, cancers include, but are not limited to, mesothelioma, leukemias and lymphomas such as cutaneous T-cell lymphomas (CTCL), noncutaneous peripheral T-cell lymphomas, lymphomas associated with human T-cell lymphotrophic virus (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodisplastic syndrome, childhood solid tumors such as brain tumors, neuroblastoma, retinoblastoma, Wilms' tumor, bone tumors, and soft-tissue sarcomas, common solid tumors of adults such as head and neck cancers, such as oral, laryngeal, nasopharyngeal and esophageal, genitourinary cancers, such as prostate, bladder, renal, uterine, ovarian, testicular, lung cancer, such as small-cell and non-small cell, breast cancer, pancreatic cancer, melanoma and other skin cancers, stomach cancer, brain tumors, tumors related to Gorlin's syndrome, such as medulloblastoma or meningioma, and liver cancer.

Additional exemplary forms of cancer include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

Additional cancers that the compounds described herein may be useful in preventing, treating and studying are, for example, colon carcinoma, familiary adenomatous polyposis carcinoma and hereditary non-polyposis colorectal cancer, or melanoma. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma. In one aspect of the application, the present application provides for the use of one or more compound as described herein, in the manufacture of a medicament for the treatment of cancer, including without limitation the various types of cancer disclosed herein. In some embodiments, the compounds of this application are useful for treating cancer, such as colorectal, thyroid, breast, and lung cancer; and myeloproliferative disorders, such as polycythemia vera, thrombocythemia, myeloid metaplasia with myelofibrosis, chronic myelogenous leukemia, chronic myelomonocytic leukemia, hypereosinophilic syndrome, juvenile myelomonocytic leukemia, and systemic mast cell disease. In some embodiments, the compound as described herein is useful for treating hematopoietic disorders, in particular, acute-myelogenous leukemia (AML), chronic-myelogenous leukemia (CML), acute-promyelocytic leukemia, and acute lymphocytic leukemia (ALL). In one embodiment, a compound or its corresponding pharmaceutically acceptable salt, or isotopic derivative, as described herein can be used in an effective amount to treat a host, for example a human, with a lymphoma or lymphocytic or myelocytic proliferation disorder or abnormality. For example, a compound as described herein can be administered to a host suffering from a Hodgkin's Lymphoma or a Non-Hodgkin's Lymphoma. For example, the host can be suffering from a Non-Hodgkin's Lymphoma such as, but not limited to: an AIDS-Related Lymphoma; Anaplastic Large-Cell Lymphoma; Angioimmunoblastic Lymphoma; Blastic NK-Cell Lymphoma; Burkitt's Lymphoma; Burkitt-like Lymphoma (Small Non-Cleaved Cell Lymphoma); diffuse small-cleaved cell lymphoma (DSCCL); Chronic Lymphocytic Leukemia/Small Lymphocytic Lymphoma; Cutaneous T-Cell Lymphoma; Diffuse Large B-Cell Lymphoma; Enteropathy-Type T-Cell Lymphoma; Follicular Lymphoma; Hepatosplenic Gamma-Delta T-Cell Lymphoma; Lymphoblastic Lymphoma; Mantle Cell Lymphoma; Marginal Zone Lymphoma; Nasal T-Cell Lymphoma; Pediatric Lymphoma; Peripheral T-Cell Lymphomas; Primary Central Nervous System Lymphoma; T-Cell Leukemias; Transformed Lymphomas; Treatment-Related T-Cell Lymphomas; Langerhans cell histiocytosis; or Waldenstrom's Macroglobulinemi a.

In another embodiment, a compound or its corresponding pharmaceutically acceptable salt, or isotopic derivative, as described herein can be used in an effective amount to treat a patient, for example a human, with a Hodgkin's lymphoma, such as, but not limited to: Nodular Sclerosis Classical Hodgkin's Lymphoma (CHL); Mixed Cellularity CHL; Lymphocyte-depletion CHL; Lymphocyte-rich CHL; Lymphocyte Predominant Hodgkin's Lymphoma; or Nodular Lymphocyte Predominant HL. This application further embraces the treatment or prevention of cell proliferative disorders such as hyperplasias, dysplasias and pre-cancerous lesions. Dysplasia is the earliest form of pre-cancerous lesion recognizable in a biopsy by a pathologist. The compounds may be administered for the purpose of preventing said hyperplasias, dysplasias or pre-cancerous lesions from continuing to expand or from becoming cancerous. Examples of pre-cancerous lesions may occur in skin, esophageal tissue, breast and cervical intra-epithelial tissue.

In certain embodiments a compound of the present invention is used to treat an abnormal cell proliferation such as a tumor or cancer that has a RET protein with a mutation, wherein the mutation is at one of the below listed amino acid sites. The mutation may, for example, be selected from one of the listed exemplary mutations, or may be a different mutation.

| Amino Acid Site | Exemplary Mutations |
|---|---|
| G810 | G810R, G810S, G810C, G810N |
| C634 | C634W, C634R |
| M918 | M918T |
| A883 | A883F |
| E762 | E762Q |
| G691 | G691S |
| L790 | L790F |
| R749 | R749T |
| R813 | R813Q |
| S891 | S891A |
| S904 | S904A, S904F |
| V778 | V778I |
| V804 | V804L, V804M, V804E |
| Y791 | Y791F |
| Y806 | Y806H |

In certain embodiments the RET protein has two mutations selected from the table above. In other embodiments the RET protein has three mutations selected from the table above. In other embodiments the RET protein has four or more mutations, which may optionally be selected from the table above.

In certain embodiments the tumor or cancer has a mutation in a RET protein that is a substantial or partial driver of tumor of cancer cell proliferation. In another embodiment the tumor or cancer has a RET altered protein that is not acting significantly as a driver of abnormal cell proliferation but can be used therapeutically to kill the tumor cell using a selected RET degrader as described herein.

In certain embodiments, a compound of the present invention is used to treat a tumor or cancer with a RET protein V804L mutation. In certain embodiments, a compound of the present invention is used to treat a tumor or cancer with a RET protein V804M mutation. In certain embodiments, a compound of the present invention is used to treat a tumor or cancer with a RET protein M918T mutation. In certain embodiments, a compound of the present invention is used to treat a tumor or cancer with a RET protein S891 A mutation. In certain embodiments, a compound of the present invention is used to treat a tumor or cancer with a RET protein L790F mutation. In certain embodiments, a compound of the present invention is used treat a tumor or cancer with a RET protein E768D mutation. In certain embodiments, a compound of the present invention is used treat a tumor or cancer with a RET protein C618S mutation. In certain embodiments, a compound of the present invention is used treat a tumor or cancer with a RET protein C618R mutation. In certain embodiments, a compound of the present invention is used to treat a tumor or cancer with a RET protein 634 missense. In certain embodiments, a compound of the present invention is used to treat a tumor or cancer with a RET protein C634R mutation. In certain embodiments, a compound of the present invention is used to treat a tumor or cancer with a RET protein C634Y mutation. In certain embodiments, a compound of the present invention is used to treat a tumor or cancer with a RET protein C634G mutation. In certain embodiments a compound of the present invention, or a pharmaceutically acceptable salt thereof, is used to treat an abnormal cell proliferation such as a tumor or cancer that has a RET protein with a G810R mutation.

In certain embodiments a compound of the present invention, or a pharmaceutically acceptable salt thereof, is used to treat an abnormal cell proliferation such as a tumor or cancer that has a RET protein with a G810S mutation.

In certain embodiments a compound of the present invention, or a pharmaceutically acceptable salt thereof, is used to treat an abnormal cell proliferation such as a tumor or cancer that has a RET protein with a G810C mutation.

In certain embodiments a compound of the present invention, or a pharmaceutically acceptable salt thereof, is used to treat an abnormal cell proliferation such as a tumor or cancer that has a RET protein with a C634W mutation.

In certain embodiments a compound of the present invention, or a pharmaceutically acceptable salt thereof, is used to treat an abnormal cell proliferation such as a tumor or cancer that has a RET protein with a M918T mutation.

In certain embodiments a compound of the present invention, or a pharmaceutically acceptable salt thereof, is used to treat an abnormal cell proliferation such as a tumor or cancer that has a RET protein with a V804L mutation.

In certain embodiments a compound of the present invention, or a pharmaceutically acceptable salt thereof, is used to treat an abnormal cell proliferation such as a tumor or cancer that has a RET protein with a V804M mutation.

In certain embodiments a compound of the present invention, or a pharmaceutically acceptable salt thereof, is used to treat an abnormal cell proliferation such as a tumor or cancer that has a RET protein fused to another protein, for example a fusion selected from CCDC6-RET, NCOA4-RET, KIF5B-RET, PRKARIA-RET, TRIM24-RET, TRIM33-RET, GOLGA5-RET, HOOK3-RET, KTN1-RET, ERC1-RET, MBD1-RET, TRIM27-RET, BRC-RET, FGFR10P-RET, PCM1-RET, AKAP13-RET, FKBP15-RET, SPECC1L-RET, TBL1XR1-RET, CUX1-RET, KIAA1468-RET, and KIAA1217-RET.

In certain embodiments a compound of the present invention, or a pharmaceutically acceptable salt thereof, is used to treat an abnormal cell proliferation such as a tumor or cancer that has a CCDC6-RET fusion. In certain embodiments a compound of the present invention, or a pharmaceutically acceptable salt thereof, is used to treat an abnormal cell proliferation such as a tumor or cancer that has a NCOA4-RET fusion.

In certain embodiments a compound of the present invention, or a pharmaceutically acceptable salt thereof, is used to treat an abnormal cell proliferation such as a tumor or cancer that has a KIF5B-RET fusion.

In accordance with the foregoing, the present application further provides a method for preventing or treating any of the diseases or disorders described above in a patient in need of such treatment, which method comprises administering to said patient a therapeutically effective amount of a compound as described herein, or an enantiomer, diastereomer, or stereoisomer thereof, or pharmaceutically acceptable salt, hydrate, or solvate thereof. For any of the above uses, the required dosage will vary depending on the mode of administration, the particular condition to be treated and the effect desired.

### Pharmaceutical Composition and Combination Therapy

In some embodiments, a compound of Formula I or a pharmaceutically acceptable salt thereof can be used in an effective amount, either alone or in combination, to treat a patient such as a human with a disorder as described herein or a RET mediated disorder.

The disclosed compounds described herein can be used in an effective amount alone or in combination with another compound of the present invention or another bioactive agent or second therapeutic agent to treat a patient such as a human with a disorder, including but not limited to those described herein.

The term “bioactive agent” is used to describe an agent, other than the selected compound according to the present invention, which can be used in combination or alternation with a compound of the present invention to achieve a desired result of therapy. In one embodiment, the compound of the present invention and the bioactive agent are administered in a manner that they are active in vivo during overlapping time periods, for example, have time-period overlapping Cmax, Tmax, AUC or other pharmacokinetic parameter. In another embodiment, the compound of the present invention and the bioactive agent are administered to a patient in need thereof that do not have overlapping pharmacokinetic parameter, however, one has a therapeutic impact on the therapeutic efficacy of the other. In one aspect of this embodiment, the bioactive agent is an immune modulator, including but not limited to a checkpoint inhibitor, including as non-limiting examples, a PD-1 inhibitor, PD-L1 inhibitor, PD-L2 inhibitor, CTLA-4 inhibitor, LAG-3 inhibitor, TIM-3 inhibitor, V-domain Ig suppressor of T-cell activation (VISTA) inhibitors, small molecule, peptide, nucleotide, or other inhibitor. In certain aspects, the immune modulator is an antibody, such as a monoclonal antibody. PD-1 inhibitors that blocks the interaction of PD-1 and PD-L1 by binding to the PD-1 receptor, and in turn inhibit immune suppression include, for example, nivolumab (Opdivo), pembrolizumab (Keytruda), pidilizumab, AMP-224 (AstraZeneca and MedImmune), PF-06801591 (Pfizer), MEDI0680 (AstraZeneca), PDR001 (Novartis), REGN2810 (Regeneron), SHR-12-1 (Jiangsu Hengrui Medicine Company and Incyte Corporation), TSR-042 (Tesaro), and the PD-L1/VISTA inhibitor CA-170 (Curis Inc.). PD-L1 inhibitors that block the interaction of PD-1 and PD-L1 by binding to the PD-L1 receptor, and in turn inhibits immune suppression, include for example, atezolizumab (Tecentriq), durvalumab (AstraZeneca and MedImmune), KN035 (Alphamab), and BMS-936559 (Bristol-Myers Squibb). CTLA-4 checkpoint inhibitors that bind to CTLA-4 and inhibits immune suppression include, but are not limited to, ipilimumab, tremelimumab (AstraZeneca and MedImmune), AGEN1884 and AGEN2041 (Agenus). LAG-3 checkpoint inhibitors include, but are not limited to, BMS-986016 (Bristol-Myers Squibb), GSK2831781 (GlaxoSmithKline), IMP321 (Prima BioMed), LAG525 (Novartis), and the dual PD-1 and LAG-3 inhibitor MGD013 (MacroGenics). An example of a TIM-3 inhibitor is TSR-022 (Tesaro). In certain embodiments the checkpoint inhibitor is selected from nivolumab/OPDIVO®; pembrolizumab/KEYTRUDA®; and pidilizumab/CT-011, MPDL3280A/RG7446; MEDI4736; MSB0010718C; BMS 936559, a PDL2/lg fusion protein such as AMP 224 or an inhibitor of B7-H3 (e.g., MGA271), B7-H4, BTLA, HVEM, TIM3, GAL9, LAG 3, VISTA, KIR, 2B4, CD160, CGEN-15049, CHK 1, CHK 2, A2aR, B-7 family ligands, or a combination thereof. In yet another embodiment, one of the active compounds described herein can be administered in an effective amount for the treatment of abnormal tissue of the female reproductive system such as breast, ovarian, endometrial, or uterine cancer, in combination or alternation with an effective amount of an estrogen inhibitor including, but not limited to, a SERM (selective estrogen receptor modulator), a SERD (selective estrogen receptor degrader), a complete estrogen receptor degrader, or another form of partial or complete estrogen antagonist or agonist. Partial anti-estrogens like raloxifene and tamoxifen retain some estrogen-like effects, including an estrogen-like stimulation of uterine growth, and also, in some cases, an estrogen-like action during breast cancer progression which actually stimulates tumor growth. In contrast, fulvestrant, a complete anti-estrogen, is free of estrogen-like action on the uterus and is effective in tamoxifen-resistant tumors. Non-limiting examples of anti-estrogen compounds are provided in WO 2014/19176 assigned to Astra Zeneca, WO2013/090921, WO 2014/203129, WO 2014/203132, and US2013/0178445 assigned to Olema Pharmaceuticals, and U.S. Pat. Nos. 9,078,871, 8,853,423, and 8,703,810, as well as US 2015/0005286, WO 2014/205136, and WO 2014/205138. Additional non-limiting examples of anti-estrogen compounds include: SERMS such as anordrin, bazedoxifene, broparestriol, chlorotrianisene, clomiphene citrate, cyclofenil, lasofoxifene, ormeloxifene, raloxifene, tamoxifen, toremifene, and fulvestrant; aromatase inhibitors such as aminoglutethimide, testolactone, anastrozole, exemestane, fadrozole, formestane, and letrozole; and antigonadotropins such as leuprorelin, cetrorelix, allylestrenol, chloromadinone acetate, cyproterone acetate, delmadinone acetate, dydrogesterone, medroxyprogesterone acetate, megestrol acetate, nomegestrol acetate, norethisterone acetate, progesterone, and spironolactone. Other estrogenic ligands that can be used according to the present invention are described in U.S. Pat. Nos. 4,418,068; 5,478,847; 5,393,763; and 5,457,117, WO2011/156518, U.S. Pat. Nos. 8,455,534 and 8,299,112, 9,078,871; 8,853,423; 8,703,810; US 2015/0005286; and WO 2014/205138, US2016/0175289, US2015/0258080, WO 2014/191726, WO 2012/084711; WO 2002/013802; WO 2002/004418; WO 2002/003992; WO 2002/003991; WO 2002/003990; WO 2002/003989; WO 2002/003988; WO 2002/003986; WO 2002/003977; WO 2002/003976; WO 2002/003975; WO 2006/078834; U.S. Pat. No. 6,821,989; US 2002/0128276; U.S. Pat. No. 6,777,424; US 2002/0016340; U.S. Pat. Nos. 6,326,392; 6,756,401; US 2002/0013327; U.S. Pat. Nos. 6,512,002; 6,632,834; US 2001/0056099; U.S. Pat. Nos. 6,583,170; 6,479,535; WO 1999/024027; U.S. Pat. No. 6,005,102; EP 0802184; U.S. Pat. Nos. 5,998,402; 5,780,497, 5,880,137, WO 2012/048058 and WO 2007/087684. In another embodiment, an active compounds described herein can be administered in an effective amount for the treatment of abnormal tissue of the male reproductive system such as prostate or testicular cancer, in combination or alternation with an effective amount of an androgen (such as testosterone) inhibitor including, but not limited to a selective androgen receptor modulator, a selective androgen receptor degrader, a complete androgen receptor degrader, or another form of partial or complete androgen antagonist. In one embodiment, the prostate or testicular cancer is androgen-resistant. Non-limiting examples of anti-androgen compounds are provided in WO 2011/156518 and U.S. Pat. Nos. 8,455,534 and 8,299,112. Additional non-limiting examples of anti-androgen compounds include: enzalutamide, apalutamide, cyproterone acetate, chlormadinone acetate, spironolactone, canrenone, drospirenone, ketoconazole, topilutamide, abiraterone acetate, and cimetidine. In one embodiment, the bioactive agent is an ALK inhibitor. Examples of ALK inhibitors include but are not limited to Crizotinib, Alectinib, ceritinib, TAE684 (NVP-TAE684), GSK1838705A, AZD3463, ASP3026, PF-06463922, entrectinib (RXDX-101), and AP26113. In one embodiment, the bioactive agent is an EGFR inhibitor. Examples of EGFR inhibitors include erlotinib (Tarceva), gefitinib (Iressa), afatinib (Gilotrif), rociletinib (CO-1686), osimertinib (Tagrisso), olmutinib (Olita), naquotinib (ASP8273), nazartinib (EGF816), PF-06747775 (Pfizer), icotinib (BPI-2009), neratinib (HKI-272; PB272); avitinib (AC0010), EAI045, tarloxotinib (TH-4000; PR-610), PF-06459988 (Pfizer), tesevatinib (XL647; EXEL-7647; KD-019), transtinib, WZ-3146, WZ8040, CNX-2006, and dacomitinib (PF-00299804; Pfizer). In one embodiment, the bioactive agent is an HER-2 inhibitor. Examples of HER-2 inhibitors include trastuzumab, lapatinib, ado-trastuzumab emtansine, and pertuzumab. In one embodiment, the bioactive agent is a CD20 inhibitor. Examples of CD20 inhibitors include obinutuzumab, rituximab, fatumumab, ibritumomab, tositumomab, and ocrelizumab. In one embodiment, the bioactive agent is a JAK3 inhibitor. Examples of JAK3 inhibitors include tasocitinib. In one embodiment, the bioactive agent is a BCL-2 inhibitor. Examples of BCL-2 inhibitors include venetoclax, ABT-199 (4-[4-[[2-(4-Chlorophenyl)-4,4-dimethylcyclohex-1-en-1-yl]methyl]piperazin-1-yl]-N-[[3-nitro-4-[[(tetrahydro-2H-pyran-4-yl)methyl]amino]phenyl]sulfonyl]-2-[(1H-pyrrolo[2,3-b]pyridin-5-yl)oxy]benzamide), ABT-737 (4-[4-[[2-(4-chlorophenyl)phenyl]methyl]piperazin-1-yl]-N-[4-[[(2R)-4-(dimethylamino)-1-phenylsulfanylbutan-2-yl]amino]-3-nitrophenyl]sulfonylbenzamide) (navitoclax), ABT-263 ((R)-4-(4-((4′-chloro-4,4-dimethyl-3,4,5,6-tetrahydro-[1,1′-biphenyl]-2-yl)methyl)piperazin-1-yl)-N-((4-((4-morpholino-1-(phenylthio) butan-2-yl)amino)-3 ((trifluoromethyl) sulfonyl)phenyl) sulfonyl)benzamide), GX15-070 (obatoclax mesylate, (2Z)-2-[(5Z)-5-[(3,5-dimethyl-1H-pyrrol-2-yl)methylidene]-4-methoxypyrrol-2-ylidene]indole; methanesulfonic acid))), 2-methoxy-antimycin A3, YC137 (4-(4,9-dioxo-4,9-dihydronaphtho[2,3-d]thiazol-2-ylamino)-phenyl ester), pogosin, ethyl 2-amino-6-bromo-4-(1-cyano-2-ethoxy-2-oxoethyl)-4H-chromene-3-carboxylate, Nilotinib-d3, TW-37 (N-[4-[[2-(1,1-Dimethylethyl)phenyl]sulfonyl]phenyl]-2,3,4-trihydroxy-5-[[2-(1-methylethyl)phenyl]methyl]benzamide), Apogossypolone (ApoG2), HA14-1, AT101, sabutoclax, gambogic acid, or G3139 (Oblimersen). In one embodiment, the bioactive agent is a kinase inhibitor. In one embodiment, the kinase inhibitor is selected from a phosphoinositide 3-kinase (PI3K) inhibitor, a Bruton's tyrosine kinase (BTK) inhibitor, or a spleen tyrosine kinase (Syk) inhibitor, or a combination thereof. Examples of PI3 kinase inhibitors include, but are not limited to, Wortmannin, demethoxyviridin, perifosine, idelalisib, Pictilisib, Palomid 529, ZSTK474, PWT33597, CUDC-907, and AEZS-136, duvelisib, GS-9820, BKM120, GDC-0032 (Taselisib) (2-[4-[2-(2-Isopropyl-5-methyl-1,2,4-triazol-3-yl)-5,6-dihydroimidazo[1,2-d][1,4]benzoxazepin-9-yl]pyrazol-1-yl]-2-methylpropanamide), MLN-1117 ((2R)-1-Phenoxy-2-butanyl hydrogen (S)-methylphosphonate; or Methyl(oxo) {[(2R)-1-phenoxy-2-butanyl]oxy}phosphonium)), BYL-719 ((2S)—N1-[4-Methyl-5-[2-(2,2,2-trifluoro-1,1-dimethylethyl)-4-pyridinyl]-2-thiazolyl]-1,2-pyrrolidinedicarboxamide), GSK2126458 (2,4-Difluoro-N-{2-(methyloxy)-5-[4-(4-pyridazinyl)-6-quinolinyl]-3-pyridinyl}benzenesulfonamide) (omipalisib), TGX-221 ((±)-7-Methyl-2-(morpholin-4-yl)-9-(1-phenylaminoethyl)-pyrido[1,2-a]-pyrimidin-4-one), GSK2636771 (2-Methyl-1-(2-methyl-3-(trifluoromethyl)benzyl)-6-morpholino-1H-benzo[d]imidazole-4-carboxylic acid dihydrochloride), KIN-193 ((R)-2-((1-(7-methyl-2-morpholino-4-oxo-4H-pyrido[1,2-a]pyrimidin-9-yl)ethyl)amino)benzoic acid), TGR-1202/RP5264, GS-9820 ((S)-1-(4-((2-(2-aminopyrimidin-5-yl)-7-methyl-4-mohydroxypropan-1-one), GS-1101 (5-fluoro-3-phenyl-2-([S)]-1-[9H-purin-6-ylamino]-propyl)-3H-quinazolin-4-one), AMG-319, GSK-2269557, SAR245409 (N-(4-(N-(3-((3,5-dimethoxyphenyl)amino)quinoxalin-2-yl)sulfamoyl)phenyl)-3-methoxy-4 methylbenzamide), BAY80-6946 (2-amino-N-(7-methoxy-8-(3-morpholinopropoxy)-2,3-dihydroimidazo[1,2-c]quinaz), AS 252424 (5-[1-[5-(4-Fluoro-2-hydroxy-phenyl)-furan-2-yl]-meth-(Z)-ylidene]-thiazolidine-2,4-dione), CZ 24832 (5-(2-amino-8-fluoro-[1,2,4]triazolo[1,5-a]pyridin-6-yl)-N-tert-butylpyridine-3-sulfonamide), Buparlisib (5-[2,6-Di(4-morpholinyl)-4-pyrimidinyl]-4-(trifluoromethyl)-2-pyridinamine), GDC-0941 (2-(1H-Indazol-4-yl)-6-[[4-(methylsulfonyl)-1-piperazinyl]methyl]-4-(4-morpholinyl) thieno[3,2-d]pyrimidine), GDC-0980 ((S)-1-(4-((2-(2-aminopyrimidin-5-yl)-7-methyl-4-morpholinothieno[3,2-d]pyrimidin-6 yl)methyl)piperazin-1-yl)-2-hydroxypropan-1-one (also known as RG7422)), SF1126 ((8S,14S,17S)-14-(carboxymethyl)-8-(3-guanidinopropyl)-17-(hydroxymethyl)-3,6,9,12,15-pentaoxo-1-(4-(4-oxo-8-phenyl-4H-chromen-2-yl) morpholino-4-ium)-2-oxa-7,10,13,16-tetraazaoctadecan-18-oate), PF-05212384 (N-[4-[[4-(Dimethylamino)-1-piperidinyl]carbonyl]phenyl]-N′-[4-(4,6-di-4-morpholinyl-1,3,5-triazin-2-yl)phenyl]urea) (gedatolisib), LY3023414, BEZ235 (2-Methyl-2-{4-[3-methyl-2-oxo-8-(quinolin-3-yl)-2,3-dihydro-1H-imidazo[4,5-c]quinolin-1-yl]phenyl}propanenitrile) (dactolisib), XL-765 (N-(3-(N-(3-(3,5-dimethoxyphenylamino)quinoxalin-2-yl)sulfamoyl)phenyl)-3-methoxy-4-methylbenzamide), and GSK1059615 (5-[[4-(4-Pyridinyl)-6-quinolinyl]methylene]-2,4-thiazolidenedione), PX886 ([(3aR,6E,9S,9aR,10R,11aS)-6-[[bis(prop-2-enyl)amino]methylidene]-5-hydroxy-9-(methoxymethyl)-9a, 11a-dimethyl-1,4,7-trioxo-2,3,3a,9,10,11-hexahydroindeno[4.5 h]isochromen-10-yl]acetate (also known as sonolisib)), LY294002, AZD8186, PF-4989216, pilaralisib, GNE-317, PI-3065, PI-103, NU7441 (KU-57788), HS 173, VS-5584 (SB2343), CZC24832, TG100-115, A66, YM201636, CAY10505, PIK-75, PIK-93, AS-605240, BGT226 (NVP-BGT226), AZD6482, voxtalisib, alpelisib, IC-87114, TGI100713, CH5132799, PKI-402, copanlisib (BAY 80-6946), XL 147, PIK-90, PIK-293, PIK-294, 3-MA (3-methyladenine), AS-252424, AS-604850, apitolisib (GDC-0980; RG7422). Examples of BTK inhibitors include ibrutinib (also known as PCI-32765) (Imbruvica™) (1-[(3R)-3-[4-amino-3-(4-phenoxy-phenyl) pyrazolo[3,4-d]pyrimidin-1-yl]piperidin-1-yl]prop-2-en-1-one), dianilinopyrimidine-based inhibitors such as AVL-101 and AVL-291/292 (N-(3-((5-fluoro-2-((4-(2-methoxyethoxy)phenyl)amino)pyrimidin-4-yl)amino)phenyl) acrylamide) (Avila Therapeutics) (see US Patent Publication No 2011/0117073, incorporated herein in its entirety), Dasatinib ([N-(2-chloro-6-methylphenyl)-2-(6-(4-(2-hydroxyethyl)piperazin-1-yl)-2-methylpyrimidin-4-ylamino) thiazole-5-carboxamide], LFM-A13 (alpha-cyano-beta-hydroxy-beta-methyl-N-(2,5-ibromophenyl) propenamide), GDC-0834 ([R—N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide], CGI-560 4-(tert-butyl)-N-(3-(8-(phenylamino)imidazo[1,2-a]pyrazin-6-yl)phenyl)benzamide, CGI-1746 (4-(tert-butyl)-N-(2-methyl-3-(4-methyl-6-((4-(morpholine-4-carbonyl)phenyl)amino)-5-oxo-4,5-dihydropyrazin-2-yl)phenyl)benzamide), CNX-774 (4-(4-((4-((3-acrylamidophenyl)amino)-5-fluoropyrimidin-2-yl)amino) phenoxy)-N-methylpicolinamide), CTA056 (7-benzyl-1-(3-(piperidin-1-yl) propyl)-2-(4-(pyridin-4-yl)phenyl)-1H-imidazo[4,5-g]quinoxalin-6 (5H)-one), GDC-0834 ((R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), GDC-0837 ((R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), HM-71224, ACP-196, ONO-4059 (Ono Pharmaceuticals), PRT062607 (4-((3-(2H-1,2,3-triazol-2-yl)phenyl)amino)-2-(((1R,2S)-2-aminocyclohexyl)amino)pyrimidine-5-carboxamide hydrochloride), QL-47 (1-(1-acryloylindolin-6-yl)-9-(1-methyl-1H-pyrazol-4-yl)benzo[h][1,6]naphthyridin-2 (1H)-one), and RN486 (6-cyclopropyl-8-fluoro-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one), and other molecules capable of inhibiting BTK activity, for example those BTK inhibitors disclosed in Akinleye et ah, Journal of Hematology & Oncology, 2013, 6:59, the entirety of which is incorporated herein by reference. Syk inhibitors include, but are not limited to, Cerdulatinib (4-(cyclopropylamino)-2-((4-(4-(ethylsulfonyl)piperazin-1-yl)phenyl)amino)pyrimidine-5-carboxamide), entospletinib (6-(1H-indazol-6-yl)-N-(4-morpholinophenyl)imidazo[1,2-a]pyrazin-8-amine), fostamatinib ([6-({5-Fluoro-2-[(3,4,5-trimethoxyphenyl)amino]-4-pyrimidinyl}amino)-2,2-dimethyl-3-oxo-2,3-dihydro-4H-pyrido[3,2-b][1,4]oxazin-4-yl]methyl dihydrogen phosphate), fostamatinib disodium salt (sodium (6-((5-fluoro-2-((3,4,5-trimethoxyphenyl)amino)pyrimidin-4-yl)amino)-2,2-dimethyl-3-oxo-2H-pyrido[3,2-b][1,4]oxazin-4 (3H)-yl)methyl phosphate), BAY 61-3606 (2-(7-(3,4-Dimethoxyphenyl)-imidazo[1,2-c]pyrimidin-5-ylamino)-nicotinamide HCl), RO9021 (6-[(1R,2S)-2-Amino-cyclohexylamino]-4-(5,6-dimethyl-pyridin-2-ylamino)-pyridazine-3-carboxylic acid amide), imatinib (Gleevac; 4-[(4-methylpiperazin-1-yl)methyl]-N-(4-methyl-3-{[4-(pyridin-3-yl)pyrimidin-2-yl]amino}phenyl)benzamide), staurosporine, GSK143 (2-(((3R,4R)-3-aminotetrahydro-2H-pyran-4-yl)amino)-4-(p-tolylamino)pyrimidine-5-carboxamide), PP2 (1-(tert-butyl)-3-(4-chlorophenyl)-1H-pyrazolo[3,4-d]pyrimidin-4-amine), PRT-060318 (2-(((1R,2S)-2-aminocyclohexyl)amino)-4-(m-tolylamino)pyrimidine-5-carboxamide), PRT-062607 (4-((3-(2H-1,2,3-triazol-2-yl)phenyl)amino)-2-(((1R,2S)-2-aminocyclohexyl)amino)pyrimidine-5-carboxamide hydrochloride), R112 (3,3′-((5-fluoropyrimidine-2,4-diyl)bis(azanediyl))diphenol), R348 (3-Ethyl-4-methylpyridine), R406 (6-((5-fluoro-2-((3,4,5-trimethoxyphenyl)amino)pyrimidin-4-yl)amino)-2,2-dimethyl-2H-pyrido[3,2-b][1,4]oxazin-3 (4H)-one), piceatannol (3-Hydroxyresveratol), YM193306 (see Singh et al. Discovery and Development of Spleen Tyrosine Kinase (SYK) Inhibitors, J. Med. Chem. 2012, 55, 3614-3643), 7-azaindole, piceatannol, ER-27319 (see Singh et al. Discovery and Development of Spleen Tyrosine Kinase (SYK) Inhibitors, J. Med. Chem. 2012, 55, 3614-3643 incorporated in its entirety herein), Compound D (see Singh et al. Discovery and Development of Spleen Tyrosine Kinase (SYK) Inhibitors, J. Med. Chem. 2012, 55, 3614-3643 incorporated in its entirety herein), PRT060318 (see Singh et al. Discovery and Development of Spleen Tyrosine Kinase (SYK) Inhibitors, J. Med. Chem. 2012, 55, 3614-3643 incorporated in its entirety herein), luteolin (see Singh et al. Discovery and Development of Spleen Tyrosine Kinase (SYK) Inhibitors, J. Med. Chem. 2012, 55, 3614-3643 incorporated in its entirety herein), apigenin (see Singh et al. Discovery and Development of Spleen Tyrosine Kinase (SYK) Inhibitors, J. Med. Chem. 2012, 55, 3614-3643 incorporated in its entirety herein), quercetin (see Singh et al. Discovery and Development of Spleen Tyrosine Kinase (SYK) Inhibitors, J. Med. Chem. 2012, 55, 3614-3643 incorporated in its entirety herein), fisetin (see Singh et al. Discovery and Development of Spleen Tyrosine Kinase (SYK) Inhibitors, J. Med. Chem. 2012, 55, 3614-3643 incorporated in its entirety herein), myricetin (see Singh et al. Discovery and Development of Spleen Tyrosine Kinase (SYK) Inhibitors, J. Med. Chem. 2012, 55, 3614-3643 incorporated in its entirety herein), morin (see Singh et al. Discovery and Development of Spleen Tyrosine Kinase (SYK) Inhibitors, J. Med. Chem. 2012, 55, 3614-3643 incorporated in its entirety herein). In one embodiment, the bioactive agent is a MEK inhibitor. MEK inhibitors are well known, and include, for example, trametinib/GSK1120212 (N-(3-{3-Cyclopropyl-5-[(2-fluoro-4-iodophenyl)amino]-6,8-dimethyl-2,4,7-trioxo-3,4,6,7-tetrahydropyrido[4,3-d]pyrimidin-1(2H-yl}phenyl) acetamide), selumetinib (6-(4-bromo-2-chloroanilino)-7-fluoro-N-(2-hydroxyethoxy)-3-methylbenzimidazole-5-carboxamide), pimasertib/AS703026/MSC 1935369 ((S)—N-(2,3-dihydroxypropyl)-3-((2-fluoro-4-iodophenyl)amino) isonicotinamide), XL-518/GDC-0973 (1-({3,4-difluoro-2-[(2-fluoro-4-iodophenyl)amino]phenyl}carbonyl)-3-[(2S)-piperidin-2-yl]azetidin-3-ol), refametinib/BAY869766/RDEAl 19 (N-(3,4-difluoro-2-(2-fluoro-4-iodophenylamino)-6-methoxyphenyl)-1-(2,3-dihydroxypropyl)cyclopropane-1-sulfonamide), PD-0325901 (N-[(2R)-2,3-Dihydroxypropoxy]-3,4-difluoro-2-[(2-fluoro-4-iodophenyl)amino]-benzamide), TAK733 ((R)-3-(2,3-Dihydroxypropyl)-6-fluoro-5-(2-fluoro-4-iodophenylamino)-8-methylpyrido[2,3-d]pyrimidine-4,7(3H,8H)-dione), MEK162/ARRY438162 (5-[(4-Bromo-2-fluorophenyl)amino]-4-fluoro-N-(2-hydroxyethoxy)-1-methyl-1H-benzimidazole-6-carboxamide), R05126766 (3-[[3-Fluoro-2-(methylsulfamoylamino)-4-pyridyl]methyl]-4-methyl-7-pyrimidin-2-yloxychromen-2-one), WX-554, R04987655/CH4987655 (3,4-difluoro-2-((2-fluoro-4-iodophenyl)amino)-N-(2-hydroxyethoxy)-5-((3-oxo-1,2-oxazinan-2yl)methyl)benzamide), or AZD8330 (2-((2-fluoro-4-iodophenyl)amino)-N-(2 hydroxyethoxy)-1,5-dimethyl-6-oxo-1,6-dihydropyridine-3-carboxamide), U0126-EtOH, PD184352 (CI-1040), GDC-0623, BI-847325, cobimetinib, PD98059, BIX 02189, BIX 02188, binimetinib, SL-327, TAK-733, PD318088. In one embodiment, the bioactive agent is a Raf inhibitor. Raf inhibitors are known and include, for example, Vemurafinib (N-[3-[[5-(4-Chlorophenyl)-1H-pyrrolo[2,3-b]pyridin-3-yl]carbonyl]-2,4-difluorophenyl]-1-propanesulfonamide), sorafenib tosylate (4-[4-[[4-chloro-3-(trifluoromethyl)phenyl]carbamoylamino]phenoxy]-N-methylpyridine-2-carboxamide; 4-methylbenzenesulfonate), AZ628 (3-(2-cyanopropan-2-yl)-N-(4-methyl-3-(3-methyl-4-oxo-3,4-dihydroquinazolin-6-ylamino)phenyl)benzamide), NVP-BHG712 (4-methyl-3-(1-methyl-6-(pyridin-3-yl)-1H-pyrazolo[3,4-d]pyrimidin-4-ylamino)-N-(3-(trifluoromethyl)phenyl)benzamide), RAF-265 (1-methyl-5-[2-[5-(trifluoromethyl)-1H-imidazol-2-yl]pyridin-4-yl]oxy-N-[4-(trifluoromethyl)phenyl]benzimidazol-2-amine), 2-Bromoaldisine (2-Bromo-6,7-dihydro-1H,5H-pyrrolo[2,3-c]azepine-4,8-dione), Raf Kinase Inhibitor IV (2-chloro-5-(2-phenyl-5-(pyridin-4-yl)-1H-imidazol-4-yl) phenol), Sorafenib N-Oxide (4-[4-[[[[4-Chloro-3 (trifluoroMethyl)phenyl]aMino]carbonyl]aMino]phenoxy]-N-Methyl-2pyridinecarboxaMide 1-Oxide), PLX-4720, dabrafenib (GSK2118436), GDC-0879, RAF265, AZ 628, SB590885, ZM336372, GW5074, TAK-632, CEP-32496, LY3009120, and GX818 (Encorafenib). In one embodiment, the bioactive agent is an AKT inhibitor, including, but not limited to, MK-2206, GSK690693, Perifosine, (KRX-0401), GDC-0068, Triciribine, AZD5363, Honokiol, PF-04691502, and Miltefosine, a FLT-3 inhibitor, including, but not limited to, P406, Dovitinib, Quizartinib (AC220), Amuvatinib (MP-470), Tandutinib (MLN518), ENMD-2076, and KW-2449, or a combination thereof. In one embodiment, the bioactive agent is an mTOR inhibitor. Examples of mTOR inhibitors include, but are not limited to, rapamycin and its analogs, everolimus (Afinitor), temsirolimus, ridaforolimus, sirolimus, and deforolimus. Examples of MEK inhibitors include but are not limited to tametinib/GSK1120212 (N-(3-{3-Cyclopropyl-5-[(2-fluoro-4-iodophenyl)amino]-6,8-dimethyl-2,4,7-trioxo-3,4,6,7-tetrahydropyrido[4,3-d]pyrimidin-1(2H-yl}phenyl) acetamide), selumetinob (6-(4-bromo-2-chloroanilino)-7-fluoro-N-(2-hydroxyethoxy)-3-methylbenzimidazole-5-carboxamide), pimasertib/AS703026/MSC1935369 ((S)—N-(2,3-dihydroxypropyl)-3-((2-fluoro-4-iodophenyl)amino) isonicotinamide), XL-518/GDC-0973 (1-({3,4-difluoro-2-[(2-fluoro-4-iodophenyl)amino]phenyl}carbonyl)-3-[(2S)-piperidin-2-yl]azetidin-3-ol) (cobimetinib), refametinib/BAY869766/RDEA119 (N-(3,4-difluoro-2-(2-fluoro-4-iodophenylamino)-6-methoxyphenyl)-1-(2,3-dihydroxypropyl)cyclopropane-1-sulfonamide), PD-0325901 (N-[(2R)-2,3-Dihydroxypropoxy]-3,4-difluoro-2-[(2-fluoro-4-iodophenyl)amino]-benzamide), TAK733 ((R)-3-(2,3-Dihydroxypropyl)-6-fluoro-5-(2-fluoro-4-iodophenylamino)-8-methylpyrido[2,3d]pyrimidine-4,7(3H,8H)-dione), MEK162/ARRY438162 (5-[(4-Bromo-2-fluorophenyl)amino]-4-fluoro-N-(2-hydroxyethoxy)-1-methyl-1H-benzimidazole-6 carboxamide), R05126766 (3-[[3-Fluoro-2-(methylsulfamoylamino)-4-pyridyl]methyl]-4-methyl-7-pyrimidin-2-yloxychromen-2-one), WX-554, R04987655/CH4987655 (3,4-difluoro-2-((2-fluoro-4-iodophenyl)amino)-N-(2-hydroxyethoxy)-5-((3-oxo-1,2-oxazinan-2 yl)methyl)benzamide), or AZD8330 (2-((2-fluoro-4-iodophenyl)amino)-N-(2-hydroxyethoxy)-1,5-dimethyl-6-oxo-1,6-dihydropyridine-3-carboxamide). In one embodiment, the bioactive agent is a RAS inhibitor. Examples of RAS inhibitors include but are not limited to Reolysin and siG12D LODER. In one embodiment, the bioactive agent is a HSP inhibitor. HSP inhibitors include but are not limited to Geldanamycin or 17-N-Allylamino-17-demethoxygeldanamycin (17AAG), and Radicicol. Additional bioactive compounds include, for example, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an aurora kinase inhibitor, a PIK-1 modulator, an HDAC inhibitor, a c-MET inhibitor, a PARP inhibitor, a Cdk inhibitor, an IGFR-TK inhibitor, an anti-HGF antibody, a focal adhesion kinase inhibitor, a Map kinase (mek) inhibitor, a VEGF trap antibody, pemetrexed, panitumumab, amrubicin, oregovomab, Lep-etu, nolatrexed, azd2171, batabulin, of atumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR1 KRX-0402, lucanthone, LY317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258); 3-[5-(methylsulfonylpiperadinemethyl)-indolyl-quinolone, vatalanib, AG-013736, AVE-0005, goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, adriamycin, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gleevec, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonist, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof. In one embodiment, the bioactive agent is selected from, but are not limited to, Imatinib mesylate (Gleevac®), Dasatinib (Sprycel®), Nilotinib (Tasigna®), Bosutinib (Bosulif®), Trastuzumab (Herceptin®), trastuzumab-DM1, Pertuzumab (Perjeta™), Lapatinib (Tykerb®), Gefitinib (Iressa®), Erlotinib (Tarceva®), Cetuximab (Erbitux®), Panitumumab (Vectibix®), Vandetanib (Caprelsa®), Vemurafenib (Zelboraf®), Vorinostat (Zolinza®), Romidepsin (Istodax®), Bexarotene (Tagretin®), Alitretinoin (Panretin®), Tretinoin (Vesanoid®), Carfilizomib (Kyprolis™), Pralatrexate (Folotyn®), Bevacizumab (Avastin®), Ziv-aflibercept (Zaltrap®), Sorafenib (Nexavar®), Sunitinib (Sutent®), Pazopanib (Votrient®), Regorafenib (Stivarga®), and Cabozantinib (Cometriq™). In certain aspects, the bioactive agent is an anti-inflammatory agent, a chemotherapeutic agent, a radiotherapeutic, an additional therapeutic agent, or an immunosuppressive agent. Suitable chemotherapeutic bioactive agents include, but are not limited to, a radioactive molecule, a toxin, also referred to as cytotoxin or cytotoxic agent, which includes any agent that is detrimental to the viability of cells, and liposomes or other vesicles containing chemotherapeutic compounds. General anticancer pharmaceutical agents include: Vincristine (Oncovin®) or liposomal vincristine (Marqibo®), Daunorubicin (daunomycin or Cerubidine®) or doxorubicin (Adriamycin®), Cytarabine (cytosine arabinoside, ara-C, or Cytosar®), L-asparaginase (Elspar®) or PEG-L-asparaginase (pegaspargase or Oncaspar®), Etoposide (VP-16), Teniposide (Vumon®), 6-mercaptopurine (6-MP or Purinethol®), Methotrexate, Cyclophosphamide (Cytoxan®), Prednisone, Dexamethasone (Decadron), imatinib (Gleevec®), dasatinib (Sprycel®), nilotinib (Tasigna®), bosutinib (Bosulif®), and ponatinib (Iclusig™). Examples of additional suitable chemotherapeutic agents include, but are not limited to 1-dehydrotestosterone, 5-fluorouracil decarbazine, 6-mercaptopurine, 6-thioguanine, actinomycin D, adriamycin, aldesleukin, an alkylating agent, allopurinol sodium, altretamine, amifostine, anastrozole, anthramycin (AMC)), an anti-mitotic agent, cis-dichlorodiamine platinum (II) (DDP) cisplatin), diamino dichloro platinum, anthracycline, an antibiotic, an antimetabolite, asparaginase, BCG live (intravesical), betamethasone sodium phosphate and betamethasone acetate, bicalutamide, bleomycin sulfate, busulfan, calcium leucouorin, calicheamicin, capecitabine, carboplatin, lomustine (CCNU), carmustine (BSNU), Chlorambucil, Cisplatin, Cladribine, Colchicin, conjugated estrogens, Cyclophosphamide, Cyclothosphamide, Cytarabine, Cytarabine, cytochalasin B, Cytoxan, Dacarbazine, Dactinomycin, dactinomycin (formerly actinomycin), daunirubicin HCL, daunorucbicin citrate, denileukin diftitox, Dexrazoxane, Dibromomannitol, dihydroxy anthracin dione, Docetaxel, dolasetron mesylate, doxorubicin HCL, dronabinol, E. coli L-asparaginase, emetine, epoetin-α, Erwinia L-asparaginase, esterified estrogens, estradiol, estramustine phosphate sodium, ethidium bromide, ethinyl estradiol, etidronate, etoposide citrororum factor, etoposide phosphate, filgrastim, floxuridine, fluconazole, fludarabine phosphate, fluorouracil, flutamide, folinic acid, gemcitabine HCL, glucocorticoids, goserelin acetate, gramicidin D, granisetron HCL, hydroxyurea, idarubicin HCL, ifosfamide, interferon α-2b, irinotecan HCL, letrozole, leucovorin calcium, leuprolide acetate, levamisole HCL, lidocaine, lomustine, maytansinoid, mechlorethamine HCL, medroxyprogesterone acetate, megestrol acetate, melphalan HCL, mercaptipurine, mesna, methotrexate, methyltestosterone, mithramycin, mitomycin C, mitotane, mitoxantrone, nilutamide, octreotide acetate, ondansetron HCL, paclitaxel, pamidronate disodium, pentostatin, pilocarpine HCL, plimycin, polifeprosan 20 with carmustine implant, porfimer sodium, procaine, procarbazine HCL, propranolol, rituximab, sargramostim, streptozotocin, tamoxifen, taxol, teniposide, tenoposide, testolactone, tetracaine, thioepa chlorambucil, thioguanine, thiotepa, topotecan HCL, toremifene citrate, trastuzumab, tretinoin, valrubicin, vinblastine sulfate, vincristine sulfate, and vinorelbine tartrate.

In some embodiments, the compound of the present invention is administered in combination with a chemotherapeutic agent (e.g., a cytotoxic agent or other chemical compound useful in the treatment of cancer). Examples of chemotherapeutic agents include alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Also included is 5-fluorouracil (5-FU), leucovorin (LV), irenotecan, oxaliplatin, capecitabine, paclitaxel, and doxetaxel. Non-limiting examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; cally statin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancrati statin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Inti. Ed Engl. 33:183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin, including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, NJ), ABRAXANE®, cremophor-free, albumin-engineered nanoparticle formulation of paclitaxel (American Pharmaceutical Partners, Schaumberg, IL), and TAXOTERE® doxetaxel (Rhone-Poulenc Rorer, Antony, France); chloranbucil; GEMZAR® gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above. Two or more chemotherapeutic agents can be used in a cocktail to be administered in combination with the compound of the present invention. Suitable dosing regimens of combination chemotherapies are known in the ar. For example combination dosing regimes are described in Saltz et al., Proc. Am. Soc. Clin. Oncol. 18: 233a (1999) and Douillard et al., Lancet 355 (9209): 1041-1047 (2000). Additional therapeutic agents that can be administered in combination with a Compound disclosed herein can include bevacizumab, sutinib, sorafenib, 2-methoxyestradiol or 2ME2, finasunate, vatalanib, vandetanib, aflibercept, volociximab, etaracizumab (MEDI-522), cilengitide, erlotinib, cetuximab, panitumumab, gefitinib, trastuzumab, dovitinib, figitumumab, atacicept, rituximab, alemtuzumab, aldesleukine, atlizumab, tocilizumab, temsirolimus, everolimus, lucatumumab, dacetuzumab, HLL1, huN901-DM1, atiprimod, natalizumab, bortezomib, carfilzomib, marizomib, tanespimycin, saquinavir mesylate, ritonavir, nelfinavir mesylate, indinavir sulfate, belinostat, panobinostat, mapatumumab, lexatumumab, dulanermin, ABT-737, oblimersen, plitidepsin, talmapimod, P276-00, enzastaurin, tipifarnib, perifosine, imatinib, dasatinib, lenalidomide, thalidomide, simvastatin, celecoxib, bazedoxifene, AZD4547, rilotumumab, oxaliplatin (Eloxatin), PD0332991, ribociclib (LEE011), amebaciclib (LY2835219), HDM201, fulvestrant (Faslodex), exemestane (Aromasin), PIM447, ruxolitinib (INC424), BGJ398, necitumumab, pemetrexed (Alimta), and ramucirumab (IMC-1121B). In one embodiment, the additional therapy is a monoclonal antibody (MAb). Some MAbs stimulate an immune response that destroys cancer cells. Similar to the antibodies produced naturally by B cells, these MAbs may “coat” the cancer cell surface, triggering its destruction by the immune system. For example, bevacizumab targets vascular endothelial growth factor (VEGF), a protein secreted by tumor cells and other cells in the tumor's microenvironment that promotes the development of tumor blood vessels. When bound to bevacizumab, VEGF cannot interact with its cellular receptor, preventing the signaling that leads to the growth of new blood vessels. Similarly, cetuximab and panitumumab target the epidermal growth factor receptor (EGFR), and trastuzumab targets the human epidermal growth factor receptor 2 (HER-2). MAbs that bind to cell surface growth factor receptors prevent the targeted receptors from sending their normal growth-promoting signals. They may also trigger apoptosis and activate the immune system to destroy tumor cells. In one aspect of the present invention, the bioactive agent is an immunosuppressive agent. The immunosuppressive agent can be a calcineurin inhibitor, e.g. a cyclosporin or an ascomycin, e.g. Cyclosporin A (NEORAL®), FK506 (tacrolimus), pimecrolimus, a mTOR inhibitor, e.g. rapamycin or a derivative thereof, e.g. Sirolimus (RAPAMUNE®), Everolimus (Certican®), temsirolimus, zotarolimus, biolimus-7, biolimus-9, a rapalog, e.g. ridaforolimus, azathioprine, campath 1H, a SIP receptor modulator, e.g. fingolimod or an analogue thereof, an anti IL-8 antibody, mycophenolic acid or a salt thereof, e.g. sodium salt, or a prodrug thereof, e.g. Mycophenolate Mofetil (CELLCEPT®), OKT3 (ORTHOCLONE OKT3®), Prednisone, ATGAM®, THYMOGLOBULIN®, Brequinar Sodium, OKT4, T10B9.A-3A, 33B3.1, 15-deoxyspergualin, tresperimus, Leflunomide ARAVAR, CTLAI-Ig, anti-CD25, anti-IL2R, Basiliximab (SIMULECT®), Daclizumab (ZENAPAX®), mizorbine, methotrexate, dexamethasone, ISAtx-247, SDZ ASM 981 (pimecrolimus, Elidel®), CTLA41g (Abatacept), belatacept, LFA31g, etanercept (sold as Enbrel® by Immunex), adalimumab (Humira®), infliximab (Remicade®), an anti-LFA-1 antibody, natalizumab (Antegren®), Enlimomab, gavilimomab, antithymocyte immunoglobulin, siplizumab, Alefacept efalizumab, pentasa, mesalazine, asacol, codeine phosphate, benorylate, fenbufen, naprosyn, diclofenac, etodolac and indomethacin, aspirin and ibuprofen. In some embodiments, the bioactive agent is a therapeutic agent which is a biologic such a cytokine (e.g., interferon or an interleukin (e.g., IL-2)) used in cancer treatment. In some embodiments the biologic is an anti-angiogenic agent, such as an anti-VEGF agent, e.g., bevacizumab (AVASTIN®). In some embodiments the biologic is an immunoglobulin-based biologic, e.g., a monoclonal antibody (e.g., a humanized antibody, a fully human antibody, an Fc fusion protein or a functional fragment thereof) that agonizes a target to stimulate an anti-cancer response, or antagonizes an antigen important for cancer. Such agents include RITUXAN® (rituximab); ZENAPAX® (daclizumab); SIMULECT® (basiliximab); SYNAGIS® (palivizumab); REMICADE® (infliximab); HERCEPTIN® (trastuzumab); MYLOTARG® (gemtuzumab ozogamicin); CAMPATH® (alemtuzumab); ZEVALIN® (ibritumomab tiuxetan); HUMIRA® (adalimumab); XOLAIR® (omalizumab); BEXXAR® (tositumomab-1-131); RAPTIVA® (efalizumab); ERBITUX® (cetuximab); AVASTIN® (bevacizumab); TYSABRI® (natalizumab); ACTEMRA® (tocilizumab); VECTIBIX® (panitumumab); LUCENTIS® (ranibizumab); SOURIS® (eculizumab); CIMZIA® (certolizumab pegol); SIMPONI® (golimumab); ILARIS® (canakinumab); STELARA® (ustekinumab); ARZERRAR (ofatumumab); PROLIA® (denosumab); NUMAX® (motavizumab); ABTHRAX® (raxibacumab); BENLYSTA® (belimumab); YERVOY® (ipilimumab); ADCETRIS® (brentuximab vedotin); PERJETA® (pertuzumab); KADCYLA® (ado-trastuzumab emtansine); and GAZYVAR (obinutuzumab). Also included are antibody-drug conjugates. The combination therapy may include a therapeutic agent which is a non-drug treatment. For example, the compound could be administered in addition to radiation therapy, cryotherapy, hyperthermia, and/or surgical excision of tumor tissue. In certain embodiments the first and second therapeutic agents are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent. In certain embodiments the second therapeutic agent is administered on a different dosage schedule than the compound of the present invention. For example the second therapeutic agent may have a treatment holiday of 1 day, 2 days, 3 days, 4 days, 5 days, 6 days, 7 days, 8 days, 9 days, 10 days, 11 days, 12 days, 13 days, or 14 days per treatment cycle. In another embodiment the first therapeutic agent has a treatment holiday. For example the first therapeutic agent may have a treatment holiday of 1 day, 2 days, 3 days, 4 days, 5 days, 6 days, 7 days, 8 days, 9 days, 10 days, 11 days, 12 days, 13 days, or 14 days per treatment cycle. In certain embodiments both the first and second therapeutic have a treatment holiday.

### VII. Pharmaceutical Compositions

A compound of Formula I, Formula II, Formula III, Formula IV, Formula V, Formula VI, or Formula VII, or its pharmaceutically acceptable salt thereof, as described herein can be administered as the neat chemical, but is more typically administered as a pharmaceutical composition, that includes an effective amount for a patient, typically a human, in need of such treatment for any of the disorders described herein. Accordingly, the disclosure provides pharmaceutical compositions comprising an effective amount of compound or pharmaceutically acceptable salt together with at least one pharmaceutically acceptable carrier for any of the uses described herein. The pharmaceutical composition may contain a compound or salt as the only active agent, or, in an alternative embodiment, the compound and at least one additional active agent.

In general, the compositions of the disclosure will be administered in a therapeutically effective amount by any of the accepted modes of administration. Suitable dosage ranges depend upon numerous factors such as the severity of the disease to be treated, the age and relative health of the subject, the potency of the compound used, the route and form of administration, the indication towards which the administration is directed, and the preferences and experience of the medical practitioner involved. One of ordinary skill in the art of treating such diseases will be able, without undue experimentation and in reliance upon personal knowledge and the disclosure of this application, to ascertain a therapeutically effective amount of the compositions of the disclosure for a given disease.

In certain embodiments the pharmaceutical composition is in a dosage form that contains from about 0.1 mg to about 2000 mg, from about 10 mg to about 1000 mg, from about 100 mg to about 800 mg, or from about 200 mg to about 600 mg of the active compound and optionally from about 0.1 mg to about 2000 mg, from about 10 mg to about 1000 mg, from about 100 mg to about 800 mg, or from about 200 mg to about 600 mg of an additional active agent in a unit dosage form. Examples are dosage forms with at least about 0.1, 1, 5, 10, 25, 50, 100, 200, 250, 300, 400, 500, 600, 700, or 750 mg of active compound, or its salt.

In certain embodiments the patient can be treated with low dosage therapy with a compound of the present invention. For example, the pharmaceutical composition can be in a dosage form that contains from about 0.1 μg to about 2000 μg, from about 10 μg to about 1000 μg, from about 100 μg to about 800 μg, or from about 200 μg to about 600 μg of the active compound. Examples are dosage forms with at least about 0.1, 1, 5, 10, 25, 50, 100, 200, 250, 300, 400, 500, 600, 700, or 750 μg of active compound, or its salt.

In certain embodiments the dose ranges from about 0.01-100 mg/kg of patient body weight, for example at least about 0.01 mg/kg, at least about 0.05 mg/kg, at least about 0.1 mg/kg, at least about 0.5 mg/kg, at least about 1 mg/kg, at least about 1.5 mg/kg, at least about 2 mg/kg, at least about 2.5 mg/kg, at least about 3 mg/kg, at least about 3.5 mg/kg, at least about 4 mg/kg, at least about 4.5 mg/kg, at least about 5 mg/kg, at least about 10 mg/kg, at least about 15 mg/kg, at least about 20 mg/kg, at least about 25 mg/kg, at least about 30 mg/kg, at least about 35 mg/kg, at least about 40 mg/kg, at least about 45 mg/kg, at least about 50 mg/kg, at least about 55 mg/kg, at least about 60 mg/kg, at least about 65 mg/kg, at least about 70 mg/kg, at least about 75 mg/kg, at least about 80 mg/kg, at least about 85 mg/kg, at least about 90 mg/kg, at least about 95 mg/kg, or at least about 100 mg/kg.

A pharmaceutically or therapeutically effective amount of the composition will be delivered to the patient. The precise effective amount will vary from patient to patient, and will depend upon the species, age, the subject's size and health, the nature and extent of the condition being treated, recommendations of the treating physician, and the therapeutics or combination of therapeutics selected for administration. The effective amount for a given situation can be determined by routine experimentation. For purposes of the disclosure, a therapeutic amount may for example be in the range of about 0.01 mg/kg to about 250 mg/kg body weight, more typically about 0.1 mg/kg to about 10 mg/kg, in at least one dose. The subject can be administered as many doses as is required to reduce and/or alleviate the signs, symptoms, or causes of the disorder in question, or bring about any other desired alteration of a biological system. When desired, formulations can be prepared with enteric coatings adapted for sustained or controlled release administration of the active ingredient.

In some embodiments, compounds disclosed herein or used as described are administered once a day (QD), twice a day (BID), or three times a day (TID). In some embodiments, compounds disclosed herein or used as described are administered at least once a day for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 35 days, at least 45 days, at least 60 days, at least 75 days, at least 90 days, at least 120 days, at least 150 days, at least 180 days, or longer.

In certain embodiments the compound of the present invention is administered once a day, twice a day, three times a day, or four times a day. In certain embodiments the compound of the present invention is administered orally once a day. In certain embodiments the compound of the present invention is administered orally twice a day. In certain embodiments the compound of the present invention is administered orally three times a day. In certain embodiments the compound of the present invention is administered orally four times a day.

In certain embodiments the compound of the present invention is administered intravenously once a day. In certain embodiments the compound of the present invention is administered intravenously twice a day. In certain embodiments the compound of the present invention is administered intravenously three times a day. In certain embodiments the compound of the present invention is administered intravenously four times a day.

In some embodiments the compound of the present invention is administered with a treatment holiday in between treatment cycles. For example, the compound may have a treatment holiday of 1 day, 2 days, 3 days, 4 days, 5 days, 6 days, 7 days, 8 days, 9 days, 10 days, 11 days, 12 days, 13 days, or 14 days per treatment cycle.

The pharmaceutical composition may also include a molar ratio of the active compound and an additional active agent. For example, the pharmaceutical composition may contain a molar ratio of about 0.5:1, about 1:1, about 2:1, about 3:1 or from about 1.5:1 to about 4:1 of an anti-inflammatory or immunosuppressing agent.

These compositions can contain any amount of active compound that achieves the desired result, for example between 0.1 and 99 weight % (wt. %) of the compound and usually at least about 5 wt. % of the compound. Some embodiments contain from about 25 wt. % to about 50 wt. % or from about 5 wt. % to about 75 wt. % of the compound.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

In certain embodiments the compound is administered as a pharmaceutically acceptable salt. Non-limiting examples of pharmaceutically acceptable salts include: acetate, adipate, alginate, ascorbate, aspartate, benzenesulfonate, benzoate, bisulfate, borate, butyrate, camphorate, camphorsulfonate, citrate, cyclopentanepropionate, digluconate, dodecyl sulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

Thus, the composition of the disclosure can be administered as a pharmaceutical formulation including one suitable for oral (including buccal and sub-lingual), rectal, nasal, topical, transdermal, pulmonary, vaginal or parenteral (including intramuscular, intra-arterial, intrathecal, subcutaneous and intravenous), injections, inhalation or spray, intra-aortal, intracranial, subdermal, intraperitioneal, subcutaneous, or by other means of administration containing conventional pharmaceutically acceptable carriers. A typical manner of administration is oral, topical or intravenous, using a convenient daily dosage regimen which can be adjusted according to the degree of affliction.

Depending on the intended mode of administration, the pharmaceutical compositions can be in the form of solid, semi-solid or liquid dosage forms, such as, for example, tablets, suppositories, pills, capsules, powders, liquids, syrup, suspensions, creams, ointments, lotions, paste, gel, spray, aerosol, foam, or oil, injection or infusion solution, a transdermal patch, a subcutaneous patch, an inhalation formulation, in a medical device, suppository, buccal, or sublingual formulation, parenteral formulation, or an ophthalmic solution, or the like, preferably in unit dosage form suitable for single administration of a precise dosage.

Some dosage forms, such as tablets and capsules, are subdivided into suitably sized unit doses containing appropriate quantities of the active components, e.g., an effective amount to achieve the desired purpose. The compositions will include an effective amount of the selected drug in combination with a pharmaceutically acceptable carrier and, in addition, can include other pharmaceutical agents, adjuvants, diluents, buffers, and the like. Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound. Classes of carriers include, but are not limited to adjuvants, binders, buffering agents, coloring agents, diluents, disintegrants, excipients, emulsifiers, flavorants, gels, glidents, lubricants, preservatives, stabilizers, surfactants, solubilizer, tableting agents, wetting agents or solidifying material. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, petroleum jelly, lanoline, polyethylene glycols, alcohols, transdermal enhancers and vegetable oils. Optional active agents may be included in a pharmaceutical composition, which do not substantially interfere with the activity of the compound of the present invention. Some excipients include, but are not limited, to liquids such as water, saline, glycerol, polyethylene glycol, hyaluronic acid, ethanol, and the like. The compound can be provided, for example, in the form of a solid, a liquid, spray dried material, a microparticle, nanoparticle, controlled release system, etc., as desired according to the goal of the therapy. Suitable excipients for non-liquid formulations are also known to those of skill in the art. A thorough discussion of pharmaceutically acceptable excipients and salts is available in Remington's Pharmaceutical Sciences, 18th Edition (Easton, Pennsylvania: Mack Publishing Company, 1990). Additionally, auxiliary substances, such as wetting or emulsifying agents, biological buffering substances, surfactants, and the like, can be present in such vehicles. A biological buffer can be any solution which is pharmacologically acceptable, and which provides the formulation with the desired pH, i.e., a pH in the physiologically acceptable range. Examples of buffer solutions include saline, phosphate buffered saline, Tris buffered saline, Hank's buffered saline, and the like. For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, and the like, an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered can also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, and the like. Actual methods of preparing such dosage forms are known, or will be apparent, to those skilled in this art; for example, see Remington's Pharmaceutical Sciences, referenced above. In yet another embodiment provided is the use of permeation enhancer excipients including polymers such as: polycations (chitosan and its quaternary ammonium derivatives, poly-L-arginine, aminated gelatin); polyanions (N-carboxymethyl chitosan, poly-acrylic acid); and, thiolated polymers (carboxymethyl cellulose-cysteine, polycarbophil-cysteine, chitosan-thiobutylamidine, chitosan-thioglycolic acid, chitosan-glutathione conjugates). The pharmaceutical compositions/combinations can be formulated for oral administration. For oral administration, the composition will generally take the form of a tablet, capsule, a softgel capsule or can be an aqueous or nonaqueous solution, suspension or syrup. Tablets and capsules are typical oral administration forms. Tablets and capsules for oral use can include one or more commonly used carriers such as lactose and corn starch. Lubricating agents, such as magnesium stearate, are also typically added. Typically, the compositions of the disclosure can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like. When liquid suspensions are used, the active agent can be combined with any oral, nontoxic, pharmaceutically acceptable inert carrier such as ethanol, glycerol, water, and the like and with emulsifying and suspending agents. If desired, flavoring, coloring and/or sweetening agents can be added as well. Other optional components for incorporation into an oral formulation herein include, but are not limited to, preservatives, suspending agents, thickening agents, and the like.

For ocular delivery, the compound can be administered, as desired, for example, via intravitreal, intrastromal, intracameral, sub-tenon, sub-retinal, retro-bulbar, peribulbar, suprachorodial, conjunctival, subconjunctival, episcleral, periocular, transscleral, retrobulbar, posterior juxtascleral, circumcorneal, or tear duct injections, or through a mucus, mucin, or a mucosal barrier, in an immediate or controlled release fashion or via an ocular device.

Parenteral formulations can be prepared in conventional forms, either as liquid solutions or suspensions, solid forms suitable for solubilization or suspension in liquid prior to injection, or as emulsions. Typically, sterile injectable suspensions are formulated according to techniques known in the art using suitable carriers, dispersing or wetting agents and suspending agents. The sterile injectable formulation can also be a sterile injectable solution or a suspension in an acceptably nontoxic parenterally acceptable diluent or solvent. Among the acceptable vehicles and solvents that can be employed are water, Ringer's solution and isotonic sodium chloride solution. In addition, sterile, fixed oils, fatty esters or polyols are conventionally employed as solvents or suspending media. In addition, parenteral administration can involve the use of a slow release or sustained release system such that a constant level of dosage is maintained.

Parenteral administration includes intraarticular, intravenous, intramuscular, intradermal, intraperitoneal, and subcutaneous routes, and include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Administration via certain parenteral routes can involve introducing the formulations of the disclosure into the body of a patient through a needle or a catheter, propelled by a sterile syringe or some other mechanical device such as a continuous infusion system. A formulation provided by the disclosure can be administered using a syringe, injector, pump, or any other device recognized in the art for parenteral administration. Preparations according to the disclosure for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms can also contain adjuvants such as preserving, wetting, emulsifying, and dispersing agents. They can be sterilized by, for example, filtration through a bacteria retaining filter, by incorporating sterilizing agents into the compositions, by irradiating the compositions, or by heating the compositions. They can also be manufactured using sterile water, or some other sterile injectable medium, immediately before use.

Sterile injectable solutions are prepared by incorporating one or more of the compounds of the disclosure in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, typical methods of preparation are vacuum-drying and freeze-drying techniques which yield a powder of the active ingredient plus any additional desired ingredient from a previously sterile-filtered solution thereof. Thus, for example, a parenteral composition suitable for administration by injection is prepared by stirring 1.5% by weight of active ingredient in 10% by volume propylene glycol and water. The solution is made isotonic with sodium chloride and sterilized.

Alternatively, the pharmaceutical compositions of the disclosure can be administered in the form of suppositories for rectal administration. These can be prepared by mixing the agent with a suitable nonirritating excipient which is solid at room temperature but liquid at the rectal temperature and therefore will melt in the rectum to release the drug. Such materials include cocoa butter, beeswax and polyethylene glycols.

The pharmaceutical compositions of the disclosure can also be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, propellants such as fluorocarbons or nitrogen, and/or other conventional solubilizing or dispersing agents. Formulations for buccal administration include tablets, lozenges, gels and the like. Alternatively, buccal administration can be effected using a transmucosal delivery system as known to those skilled in the art. The compounds of the disclosure can also be delivered through the skin or muscosal tissue using conventional transdermal drug delivery systems, i.e., transdermal “patches” wherein the agent is typically contained within a laminated structure that serves as a drug delivery device to be affixed to the body surface. In such a structure, the drug composition is typically contained in a layer, or “reservoir,” underlying an upper backing layer. The laminated device can contain a single reservoir, or it can contain multiple reservoirs. In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like.

Alternatively, the drug-containing reservoir and skin contact adhesive are present as separate and distinct layers, with the adhesive underlying the reservoir which, in this case, can be either a polymeric matrix as described above, or it can be a liquid or gel reservoir, or can take some other form. The backing layer in these laminates, which serves as the upper surface of the device, functions as the primary structural element of the laminated structure and provides the device with much of its flexibility. The material selected for the backing layer should be substantially impermeable to the active agent and any other materials that are present.

The compositions of the disclosure can be formulated for aerosol administration, particularly to the respiratory tract and including intranasal administration. The compound may, for example generally have a small particle size for example of the order of 5 microns or less. Such a particle size can be obtained by means known in the art, for example by micronization. The active ingredient is provided in a pressurized pack with a suitable propellant such as a chlorofluorocarbon (CFC) for example dichlorodifluoromethane, tri chlorofluoromethane, or dichlorotetrafluoroethane, carbon dioxide or other suitable gas. The aerosol can conveniently also contain a surfactant such as lecithin. The dose of drug can be controlled by a metered valve.

Alternatively, the active ingredients can be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition can be presented in unit dose form for example in capsules or cartridges of e.g., gelatin or blister packs from which the powder can be administered by means of an inhaler.

Formulations suitable for rectal administration are typically presented as unit dose suppositories. These may be prepared by admixing the active compound with one or more conventional solid carriers, for example, cocoa butter, and then shaping the resulting mixture.

In certain embodiments, the pharmaceutical composition is suitable for topical application to the skin using a mode of administration and defined above.

In certain embodiments, the pharmaceutical composition is suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6): 318 (1986)) and typically take the form of an optionally buffered aqueous solution of the active compound.

In one embodiment, microneedle patches or devices are provided for delivery of drugs across or into biological tissue, particularly the skin. The microneedle patches or devices permit drug delivery at clinically relevant rates across or into skin or other tissue barriers, with minimal or no damage, pain, or irritation to the tissue.

Formulations suitable for administration to the lungs can be delivered by a wide range of passive breath driven and active power driven single/-multiple dose dry powder inhalers (DPI). The devices most commonly used for respiratory delivery include nebulizers, metered-dose inhalers, and dry powder inhalers. Several types of nebulizers are available, including jet nebulizers, ultrasonic nebulizers, and vibrating mesh nebulizers. Selection of a suitable lung delivery device depends on parameters, such as nature of the drug and its formulation, the site of action, and pathophysiology of the lung.

### Synthetic Methodology

The compounds of the present disclosure can be prepared in a number of ways well known to those skilled in the art of organic synthesis. By way of example, compounds of the present disclosure can be synthesized using the methods described below, together with synthetic methods known in the art of synthetic organic chemistry, or variations thereon as appreciated by those skilled in the art. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5th edition, John Wiley & Sons: New York, 2001; Greene, T. W., Wuts, P. G. M., Protective Groups in Organic Synthesis, 3rd edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed, Encyclopedia of Reagents for Organic Synthesis, John Wiley and Sons (1995), incorporated by reference herein, are useful and recognised reference textbooks of organic synthesis known to those in the art.

During the reaction sequences and synthetic schemes described herein, the order of certain steps may be changed, such as the introduction and removal of protecting groups. One of ordinary skill in the art will recognize that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3rd edition. John Wiley & Sons. New York, 1999.

It is to be understood that the synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers, however, it will be understood that a given isomer, tautomer, regioisomer or stereoisomer may have a higher level of activity than another isomer, tautomer, regioisomer or stereoisomer.

Methods include but are not limited to those methods described below. Compounds of the present disclosure can be synthesized by following the steps outlined in General Schemes 1, 2, and 3 which comprise different sequences of assembling intermediates or compounds. Starting materials are either commercially available or made by known procedures in the reported literature or as illustrated below.

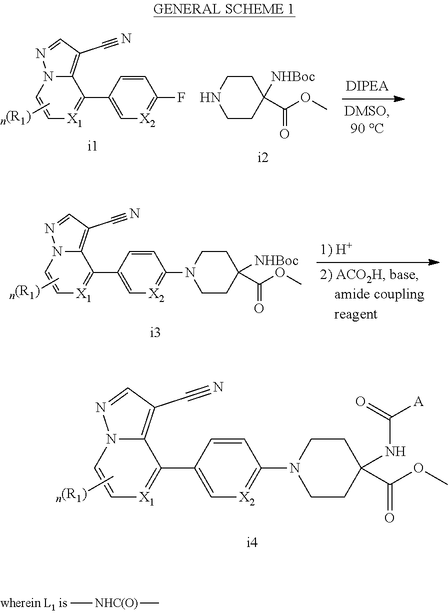

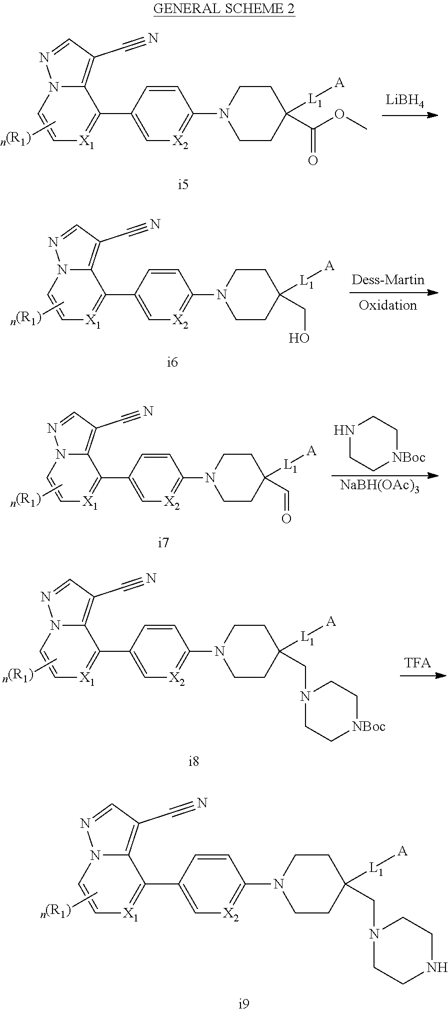

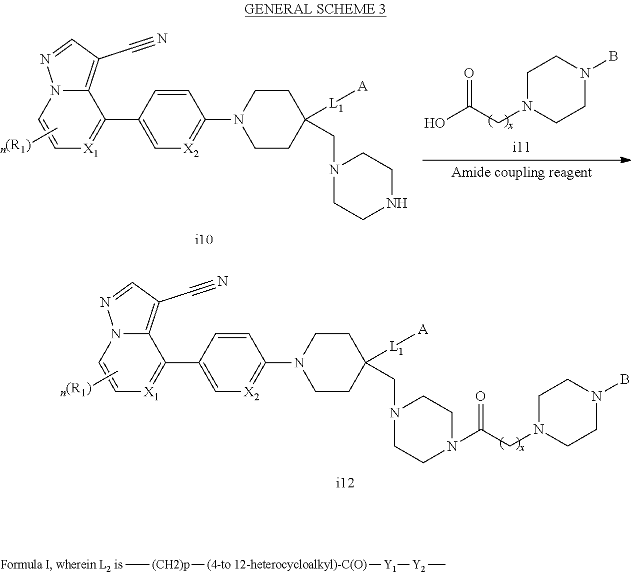

General Scheme 1: Compound i1 is coupled with compound i2 under basic conditions (e.g. DIPEA) to provide compound i3. Compound i3 is deprotected (e.g. under acidic conditions with HCl) and reacted with a carboxylic acid having moiety A, under amide coupling conditions (e.g. HATU followed by DIEA) to provide compound i4, wherein L₁ is —NHC(O).

General Scheme 2: Compound i5 is reacted with a reducing agent (e.g. LiBH₄) to provide compound i6. Compound i6 is oxidized (e.g. Dess-Martin oxidation) to provide compound i7. Compound i7 is coupled with a Boc-protected piperazine using a reductive amination reagent (e.g. sodium triacetoxyborohydride) to provide compound i8, which is deptrotected (e.g. using TFA) to provide compound i9.

General Scheme 3: Compound i10 is coupled with compound i11 using an amide coupling reagent (e.g. benzotriazol-1-yloxytris(dimethylamino)phosphonium hexafluorophosphate “BOP”) to provide compound i12, which is of Formula I, wherein L₂ is (CH₂)ₚ-(4- to 12-heterocycloalkyl)-C(O)—Y₁-Y₂—.

Various examples presented herein are synthesized according to the general procedure presented in Scheme 1, or via similar synthetic pathways.

### EXAMPLES

The compounds described herein can be made using conventional organic syntheses and commercially available starting materials, or the methods provided herein. By way of example and not limitation, compounds of formula I, can be prepared as outlined in the examples set forth herein. It should be noted that one skilled in the art would know how to modify the procedures set forth in the examples to arrive at the desired products.

### Analytical Procedures

### NMR

The following conditions were used for obtaining proton nuclear magnetic resonance (NMR) spectra: NMR spectra were taken in either 400 MHz or 500 MHz. Bruker instrument using either DMSO-d₆ or CDCl₃ as solvent and internal standard. The crude NMR data was analyzed by using either ACD Spectrus version 2015-01 by ADC Labs or MestReNova software.

Chemical shifts are reported in parts per million (ppm) downfield from internal tetramethylsilane (TMS) or from the position of TMS inferred by the deuterated NMR solvent. Apparent multiplicities are reported as: singlet—s, doublet—d, triplet—t, quartet—q, or multiplet—m. Peaks that exhibit broadening are further denoted as br. Integrations are approximate. It should be noted that integration intensities, peak shapes, chemical shifts and coupling constants can be dependent on solvent, concentration, temperature, pH, and other factors. Further, peaks that overlap with or exchange with water or solvent peaks in the NMR spectrum may not provide reliable integration intensities. In some cases, NMR spectra may be obtained using water peak suppression, which may result in overlapping peaks not being visible or having altered shape and/or integration.

### Liquid Chromatography

The following preparative and/or analytical (LC/MS) liquid chromatography methods were used.
Method A: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50 °C; Gradient: 0-100% B (0.0-3.0 min), 100% B (3.0-3.5 min); Flow: 1.0 mL/min; Detection: UV (220 nm) and MS (ESI+).
Method B: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50 °C; Gradient: 0-100% B (0.0-3.0 min), 100% B (3.0-3.5 min); Flow: 1.0 mL/min; Detection: UV (220 nm) and MS (ESI+).
Method Column 6: Column: Waters Acquity BEH C18 2.1×50 mm 1.7 μm particles;
Mobile Phase A: 95:5 acetonitrile:water with 0.05% TFA; Mobile Phase B: 95:5 acetonitrile:water with 0.05% TFA; Temperature: 50 °C; Gradient: 0% B to 100% B over 1.00 min, then a 0.50 min hold at 100% B; Flow: 1.0 mL/min; Detection: MS and UV (254 nm).
UHPLC Method D: Column: Waters Acquity BEH C18 2.1×50 mm 1.7 μm particles; Mobile Phase A: 95:5 acetonitrile:water with 0.05% TFA; Mobile Phase B: 95:5 acetonitrile:water with 0.05% TFA; Temperature: 50 °C; Gradient: 0% B to 100% B over 3.00 min, then a 0.50 min hold at 100% B; Flow: 1.0 mL/min; Detection: MS and UV (254 nm).
Method P: Mode: Binary gradient, Pump A: LC-20ADXR, Pump B: LC-20ADXR, Total Flow: 1.5000 mL/min, B Conc.: 30.0%, Oven Temperature: 40 °C, PDA Model: SPD-M20A, Lamp: D2, Start Wavelength: 190 nm, End Wavelength: 400 nm, Column Name: XBridge BEH Shield RP18, Length: 30 mm, Internal Diameter: 4.6 mm,
Description: 2.5 um particles, Mobile Phase A: Water/5 mM NH₄HCO₃, Mobile Phase B: Acetonitrile; Acquisition Mode: Scan, Polarity: Positive.
Method Q: Mode: Binary gradient, Pump A: LC-40D XR, Pump B: LC-40D XR; Oven Temperature: 40 °C; PDA Model: SPD-M20A, Lamp: D2, Start Wavelength: 190 nm, End Wavelength: 400 nm; Column Name: ACE Excel 2C18, Length: 30 mm, Internal Diameter: 3.0 mm, Column Particle Size: 2.0 μm, Mobile phase A: Water+0.05% TFA, Mobile phase B: Acetonitrile+0.05% TFA, Start Time: 0.00 min, End Time: 3.00 min, Acquisition Mode: Scan, Polarity: Positive;
LCMS5: Waters Acquity BEH C18 2.1×50 mm 1.7 μm particles; Mobile Phase A: 95:5 water:acetonitrile with 0.05% TFA; Mobile Phase B: 95:5 acetonitrile:water with 0.05% TFA; Temperature: 50 °C; Gradient: 0% B to 100% B over 2.00 min, then a 0.50 min hold at 100% B; Flow: 1.0 mL/min; Detection: MS and UV (254 nm).

### ACRONYMS AND ABBREVIATIONS

Table 1 provides a list of acronyms and abbreviations used in this specification, along with their meanings.

TABLE 1

| ACRONYM OR ABBREVIATION | MEANING OR DEFINITION |
|---|---|
| Ac | Acetyl |
| ACN, MeCN | Acetonitrile |
| Aq | Aqueous |
| Boc | t-Butyloxycarbonyl |
| BTEAC | Benzyltriethylammonium chloride |
| DBU | 1,8-Diazabicyclo[5.4.0]undec-7-ene |
| DCC | Dicyclohexyl carbodiimide |
| DCM | Dichloromethane |
| DIPEA, DIEA | N,N-diisopropylethylamine, also known as |
| | Hunig's base |
| DMA | N,N-Dimethylacetamide |
| DMAP | 4-(Dimethylamino)pyridine |
| DMF | N,N-dimethylformamide |
| DMSO | Dimethyl sulfoxide |
| EA, EtOAc | Ethyl acetate |
| h | Hour |
| HATU | Hexafluorophosphate Azabenzotriazole |
| | Tetramethyl Uronium; 1-[Bis(dimethylamino)- |
| | methylene]-1H-1,2,3-triazolo[4,5-b]pyridinium 3- |
| | oxide hexafluorophosphate |
| HPLC | High pressure liquid chromatography |
| Hunig's base | See DIPEA, DIEA |
| IBX | 2-Iodoxybenzoic acid |
| LCMS, LC-MS, LC/MS | Liquid chromatography-mass spectrometry |
| min | Minute |
| MS | Mass spectrometry |
| NMI | N-methylimidazole |
| NMM | N-methylmorpholine |
| NMO | N-Methylmorpholine N-oxide |
| NMR | Nuclear magnetic resonance |
| PE | Petroleum ether |
| Prep | Preparative |
| RT (in context of liquid | Retention time, in min |
| chromatography) | |
| rt or RT (in the context of reaction | Room (ambient) temperature, circa 25 °C |
| conditions) | |
| Sat. | Saturated |
| Soln | Solution |
| T₃P | Propanephosphonic acid anhydride |
| TCFH | N,N,N′,N-tetramethylchloroformamidinium |
| | hexafluorophosphate |
| TEA | Triethylamine |
| TFA | Trifluoroacetic acid |
| THF | Tetrahydrofuran |
| TIPT | Tetra isopropyl titanate |
| TLC | Thin layer chromatography |
| TMSCN | Trimethylsilyl cyanide |

### Example 1

### N-(1-(5-(3-cyano-6-ethoxypyrazolo[1,5-a]pyridin-4-yl)pyridin-2-yl)-4-((4-(7-(4-(2-(2,6-dioxopiperidin-3-yl)-1-oxoisoindolin-5-yl)piperazin-1-yl)heptanoyl)piperazin-1-yl)methyl)piperidin-4-yl)-2,5-difluorobenzamide

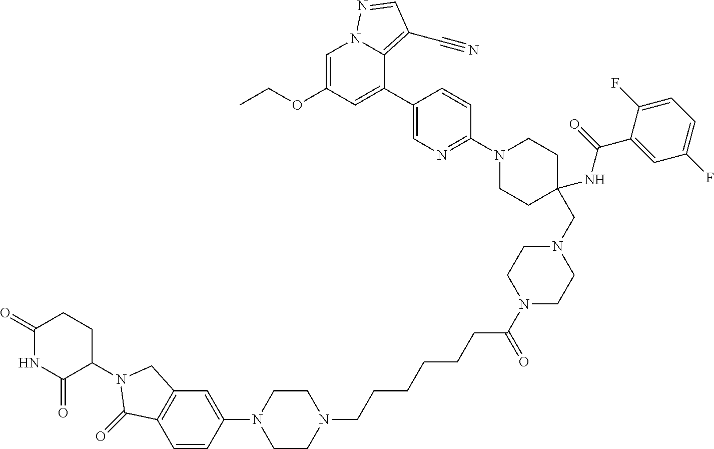 [SMILES: CCOc1cc(-c2ccc(N3CCC(CN4CCN(C(=O)CCCCCCN5CCN(c6ccc7c(c6)CN(C6CCC(=O)NC6=O)C7=O)CC5)CC4)(NC(=O)c4cc(F)ccc4F)CC3)nc2)c2c(C#N)cnn2c1]

### A. Methyl 4-((tert-butoxycarbonyl)amino)-1-(5-(3-cyano-6-ethoxypyrazolo[1,5-a]pyridin-4-yl)pyridin-2-yl)piperidine-4-carboxylate

To a sealable reaction vial containing 6-ethoxy-4-(6-fluoropyridin-3-yl) pyrazolo[1,5-a]pyridine-3-carbonitrile (1.55 g, 5.49 mmol) and methyl 4-((tert-butoxycarbonyl)amino)piperidine-4-carboxylate (2.00 g, 7.36 mmol), in anhydrous DMSO (11 ml), was added diisopropylethylamine (3.00 ml, 17.18 mmol). The vial was sealed, and the mixture was stirred at 90 °C for 16 hours. The mixture was cooled to room temperature before methyl 4-((tert-butoxycarbonyl)amino)piperidine-4-carboxylate (1.00 g, 3.68 mmol) and diisopropylethylamine (1.50 mL, 8.59 mmol) were added and the mixture was stirred at 90 °C for an additional 24 hours. After cooling to room temperature, and the mixture was partitioned between EtOAc and H₂O. The layers were separated, and the aqueous layer was extracted with EtOAc. The organic extracts were combined, washed with brine, dried (anhydrous Na₂SO₄), filtered and concentrated in vacuo to afford the crude product as an oil. Purification by flash chromatography on silica gel afforded the desired product as a pale-yellow solid (2.63 g, 90% yield). ¹H NMR (400 MHz, CHLOROFORM-d) δ 8.35-8.30 (m, 1H), 8.18 (s, 1H), 8.10 (d, J=2.1 Hz, 1H), 7.71 (dd, J=8.8, 2.5 Hz, 1H), 7.09 (d, J=2.1 Hz, 1H), 6.79 (dd, J=8.9, 0.7 Hz, 1H), 4.82 (br s, 1H), 4.13-4.03 (m, 4H), 3.76 (s, 3H), 3.38 (ddd, J=13.6, 10.6, 3.2 Hz, 2H), 2.27-2.16 (m, 2H), 2.14-2.02 (m, 2H), 1.50 (t, J=7.0 Hz, 3H), 1.45 (s, 9H). MS: m/z 521.2 [M+H]⁺ (Method Column 6).

### B. Methyl 4-amino-1-(5-(3-cyano-6-ethoxypyrazolo[1,5-a]pyridin-4-yl)pyridin-2-yl)-piperidine-4-carboxylate

To a solution of methyl 4-((tert-butoxycarbonyl)amino)-1-(5-(3-cyano-6-ethoxypyrazolo[1,5-a]pyridin-4-yl)pyridin-2-yl)piperidine-4-carboxylate (2.63 g, 5.05 mmol) in DCM (30 ml), at room temperature under nitrogen atmosphere, was added HCl (4N in dioxane, 30 ml, 120 mmol) dropwise via syringe. The resulting mixture was stirred at ambient temperature for five hours before being concentrated in vacuo to remove volatiles. The residue was treated with MeCN, frozen, then lyophilized overnight to afford the desired product as a tris HCl salt, as an off-white solid (2.83 g, 100% yield) which was used without further purification. ¹H NMR (400 MHz, DMSO-d₆) δ 8.68 (d, J=2.1 Hz, 1H), 8.58 (s, 1H), 8.35 (d, J=2.5 Hz, 1H), 7.89 (dd, J=8.9, 2.4 Hz, 1H), 7.31 (d, J=2.0 Hz, 1H), 7.11 (br d, J=9.1 Hz, 1H), 4.16 (br q, J=7.0 Hz, 2H), 4.06-3.97 (m, 2H), 3.81 (s, 3H), 3.76-3.64 (m, 3H), 3.53-3.44 (m, 1H), 2.18 (ddd, J=13.6, 6.9, 3.8 Hz, 2H), 2.00-1.89 (m, 2H), 1.38 (t, J=7.0 Hz, 3H). MS: m/z 421.1 [M+H]⁺ (Method Column 6).

### C. Methyl 1-(5-(3-cyano-6-ethoxypyrazolo[1,5-a]pyridin-4-yl)pyridin-2-yl)-4-(2,5-difluoro-benzamido)piperidine-4-carboxylate

To a solution of methyl 4-amino-1-(5-(3-cyano-6-ethoxy-pyrazolo[1,5-a]pyridin-4-yl)pyridin-2-yl)piperidine-4-carboxylate, 3 HCl (2.25 g, 4.25 mmol) in DCM (40 ml), at room temperature under nitrogen, was added 2,5-difluorobenzoic acid (1.01 g, 6.38 mmol) and HATU (2.42 g, 6.38 mmol) followed by DIEA (4.71 ml, 27.00 mmol). The resulting mixture was stirred at ambient temperature for 19 hours before being concentrated to one half of its original volume then purified directly by flash chromatography on silica gel to afford the desired product as a yellow solid (1.67 g, 70% yield). ¹H NMR (400 MHz, CHLOROFORM-d) δ 8.38-8.31 (m, 1H), 8.19 (s, 1H), 8.11 (d, J=2.1 Hz, 1H), 7.77 (ddd, J=8.8, 5.9, 3.2 Hz, 1H), 7.72 (dd, J=8.9, 2.5 Hz, 1H), 7.23-7.03 (m, 4H), 6.82 (d, J=8.9 Hz, 1H), 4.22-4.13 (m, 2H), 4.09 (q, J=7.0 Hz, 2H), 3.79 (s, 3H), 3.39 (ddd, J=13.8, 10.4, 3.3 Hz, 2H), 2.40-2.30 (m, 2H), 2.29-2.20 (m, 2H), 1.50 (t, J=7.0 Hz, 3H). Analytical LCMS: m/z 561.3 [M+H]⁺, RT=1.601 min (UHPLC Method D).

### D. N-(1-(5-(3-cyano-6-ethoxypyrazolo[1,5-a]pyridin-4-yl)pyridin-2-yl)-4-(hydroxymethyl)-piperidin-4-yl)-2,5-difluorobenzamide

To a solution of methyl 1-(5-(3-cyano-6-ethoxy-pyrazolo[1,5-a]pyridin-4-yl)pyridin-2-yl)-4-(2,5-difluorobenzamido)piperidine-4-carboxylate (1.55 g, 2.76 mmol) in THF (55 ml), at 0 °C under nitrogen atmosphere, was added lithium borohydride (0.27 g, 11.05 mmol). The reaction mixture was stirred and allowed to slowly warm to room temperature overnight. After 22 hours, the reaction mixture was diluted with EtOAc then transferred to a separatory funnel where it was carefully washed with slow addition of 10% aqueous citric acid solution (ca. 10 mL). The organic layer was washed with water then brine before being dried (anhydrous Na₂SO₄), filtered and concentrated in vacuo to afford a pale-yellow solid (1.15 g, 78%) which was used without further purification. ¹H NMR (400 MHz, DMSO-d₆) δ 8.64 (d, J=2.1 Hz, 1H), 8.57 (s, 1H), 8.32 (d, J=2.6 Hz, 1H), 7.96 (s, 1H), 7.75 (dd, J=8.9, 2.5 Hz, 1H), 7.42-7.30 (m, 3H), 7.28 (d, J=2.1 Hz, 1H), 6.97 (d, J=8.6 Hz, 1H), 4.83 (t, J=5.9 Hz, 1H), 4.23-4.12 (m, 4H), 3.62 (d, J=6.0 Hz, 2H), 3.25-3.13 (m, 2H), 2.26 (br d, J=13.4 Hz, 2H), 1.67-1.54 (m, 2H), 1.38 (t, J=7.0 Hz, 3H). Analytical LCMS: m/z 533.4 [M+H]⁺, RT=1.266 min (UHPLC Method D).

### E. N-(1-(5-(3-cyano-6-ethoxypyrazolo[1,5-a]pyridin-4-yl)pyridin-2-yl)-4-formylpiperidin-4-yl)-2,5-difluorobenzamide

To a solution of N-(1-(5-(3-cyano-6-ethoxypyrazolo[1,5-a]pyridin-4-yl)pyridin-2-yl)-4-(hydroxymethyl)piperidin-4-yl)-2,5-difluorobenzamide (1.59 g, 2.99 mmol) in DCM (20 ml), at 0 °C under nitrogen atmosphere, was added Dess-Martin periodinane (1.33 g, 3.14 mmol). The reaction mixture was stirred and allowed to warm to room temperature. After 21 hours, Dess-Martin periodinane (1.33 g, 3.14 mmol) was added and stirring was continued for an additional 5.5 hours. The reaction was quenched with water then extracted with DCM. The organic layer was washed with water before being concentrated in vacuo to afford an orange solid. Trituration with DCM, followed by removal of the solid byproduct by vacuum filtration, resulted in a filtrate which was purified by flash chromatography on silica gel to afford the desired product as a cream colored solid (711.2 mg, 45% yield). MS: m/z 531.2 [M+H]⁺ (Method Column 6).

### F. Tert-butyl 4-((1-(5-(3-cyano-6-ethoxypyrazolo[1,5-a]pyridin-4-yl)pyridin-2-yl)-4-(2,5-difluorobenzamido)piperidin-4-yl)methyl)piperazine-1-carboxylate

To a solution of N-(1-(5-(3-cyano-6-ethoxypyrazolo[1,5-a]pyridin-4-yl)pyridin-2-yl)-4-formylpiperidin-4-yl)-2,5-difluorobenzamide (0.71 g, 1.34 mmol) in DCM (7.5 mL), at room temperature in a sealable reaction vial, was added tert-butyl piperazine-1-carboxylate (0.75 g, 4.02 mmol). The mixture was stirred for one hour before sodium triacetoxyborohydride (0.43 g, 2.01 mmol) was added in one portion. The resulting mixture was stirred for 15 hours before being purified directly by flash chromatography on silica gel to afford the desired product as an off-white solid (732.8 mg, 78% yield). MS: m/z 701.4 [M+H]⁺ (Method Column 6).

### G. N-(1-(5-(3-cyano-6-ethoxypyrazolo[1,5-a]pyridin-4-yl)pyridin-2-yl)-4-(piperazin-1-ylmethyl)piperidin-4-yl)-2,5-difluorobenzamide

To a solution of tert-butyl 4-((1-(5-(3-cyano-6-ethoxypyrazolo[1,5-a]pyridin-4-yl)pyridin-2-yl)-4-(2,5-difluorobenzamido)-piperidin-4-yl)methyl)piperazine-1-carboxylate (1.70 g, 2.42 mmol) in DCM (25 ml), at 0 °C under nitrogen atmosphere, was added TFA (3.70 ml, 48.0 mmol) dropwise via syringe. The ice bath was removed, and reaction was stirred at ambient temperature for 5 hours before being concentrated in vacuo to afford the crude product as an orange residue. Purification by RP Prep-HPLC afforded the desired product as a bis TFA salt (777.8 mg, 39% yield) as a white solid. ¹H NMR (400 MHz, DMSO-d₆) δ 8.66 (d, J=2.0 Hz, 1H), 8.58 (s, 1H), 8.56-8.42 (m, 2H), 8.33 (d, J=2.5 Hz, 1H), 8.20-8.08 (m, 1H), 7.80 (dd, J=8.9, 2.6 Hz, 1H), 7.42-7.32 (m, 3H), 7.28 (d, J=2.1 Hz, 1H), 7.04 (d, J=8.9 Hz, 1H), 4.24-4.13 (m, 4H), 3.24-3.14 (m, 2H), 3.12-3.01 (m, 4H), 2.88-2.70 (m, 5H), 2.36-2.27 (m, 2H), 1.70-1.58 (m, 2H), 1.38 (t, J=7.0 Hz, 3H). MS: m/z 601.3 [M+H]⁺ (Method Column 6).

### H. Tert-butyl 7-(4-(2-(2,6-dioxopiperidin-3-yl)-1-oxoisoindolin-5-yl)piperazin-1-yl)heptanoate

To a mixture of 3-(1-oxo-5-(piperazin-1-yl) isoindolin-2-yl)piperidine-2,6-dione, HCl (320.0 mg, 0.88 mmol) in MeCN (12 mL), at room temperature in a sealable reaction vial, was added tert-butyl 7-bromoheptanoate (256.0 mg, 0.97 mmol) followed by sodium iodide (32.9 mg, 0.22 mmol) and DIEA (0.23 mL, 1.32 mmol). The vial was sealed, and the mixture was stirred at 80 °C. After 15 hours, the mixture was concentrated in vacuo then purified by flash chromatography on silica gel to afford the desired product as an off-white solid (310.4 mg, 69% yield). MS: m/z 513.3 [M+H]⁺ (Method Column 6).

### I. 7-(4-(2-(2,6-Dioxopiperidin-3-yl)-1-oxoisoindolin-5-yl)piperazin-1-yl)heptanoic acid

To a mixture of tert-butyl 7-(4-(2-(2,6-dioxopiperidin-3-yl)-1-oxoisoindolin-5-yl)piperazin-1-yl)heptanoate (310.4 mg, 0.61 mmol) in DCM (15 mL), at room temperature under nitrogen, was added TFA (1.0 mL, 12.98 mmol). The reaction was stirred at ambient temperature for four hours before being concentrated in vacuo to remove volatiles. The resultant residue was treated with MeCN, frozen, then lyophilized overnight to afford the desired product as a TFA salt, as a solid (279.0 mg, 81% yield) which was used without further purification. MS: m/z 457.2 [M+H]⁺ (Method Column 6).

### J. N-(1-(5-(3-cyano-6-ethoxypyrazolo[1,5-a]pyridin-4-yl)pyridin-2-yl)-4-((4-(7-(4-(2-(2,6-dioxopiperidin-3-yl)-1-oxoisoindolin-5-yl)piperazin-1-yl)heptanoyl)piperazin-1-yl)methyl)piperidin-4-yl)-2,5-difluorobenzamide

Nov. 12, 2021 To a sealable reaction vial containing the TFA salt of 7-(4-(2-(2,6-dioxopiperidin-3-yl)-1-oxoisoindolin-5-yl)piperazin-1-yl)heptanoic acid (191.0 mg, 0.34 mmol) in DMF (3.4 mL), was added BOP (201.0 mg, 0.45 mmol). To the resultant mixture was added a solution of the bis TFA salt of N-(1-(5-(3-cyano-6-ethoxypyrazolo[1,5-a]pyridin-4-yl)pyridin-2-yl)-4-(piperazin-1-ylmethyl)piperidin-4-yl)-2,5-difluorobenzamide (198.0 mg, 0.24 mmol) in DMF (3.4 mL), followed by DIEA (0.34 mL, 1.95 mmol). The reaction vial was capped, and the reaction mixture was stirred at room temperature. After 90 hours, the reaction mixture was diluted with DMF then purified by RP Prep-HPLC to afford the titled compound (124.9 mg, 50% yield) as a white solid. 1H NMR (400 MHz, DMSO-d6) d 10.94 (br s, 1H), 8.64 (d, J=2.0 Hz, 1H), 8.57 (s, 1H), 8.32 (d, J=2.6 Hz, 1H), 8.12 (s, 1H), 7.75 (dd, J=8.8, 2.6 Hz, 1H), 7.42 (d, J=8.5 Hz, 1H), 7.38-7.31 (m, 3H), 7.28-7.23 (m, 2H), 7.15 (d, J=2.3 Hz, 1H), 6.97 (d, J=8.8 Hz, 1H), 5.09 (dd, J=13.3, 5.0 Hz, 1H), 4.38-4.29 (m, 1H), 4.26-4.12 (m, 5H), 3.41 (br d, J=2.0 Hz, 4H), 3.22-3.09 (m, 6H), 2.97-2.84 (m, 1H), 2.72 (s, 2H), 2.64-2.55 (m, 1H), 2.48-2.23 (m, 11H), 2.03-1.94 (m, 1H), 1.67-1.55 (m, 2H), 1.53-1.41 (m, 4H), 1.38 (t, J=7.0 Hz, 3H), 1.33-1.25 (m, 4H)—Integration low due to overlap with suppressed water and obscurement by DMSO signal. Analytical LCMS: m/z 1039.3 [M+H]⁺, RT=1.273 min (UHPLC Method D). Prep-HPLC purification condition: Column: Phen Axia C18 5μ 30×100 mm; Flow rate: 40 mL/min flow rate; Mobile Phase A: 95% H2O/5% MeCN with 0.01M NH4OAc. Mobile Phase B: 5% H2O/95% MeCN with 0.01M NH4OAc; Gradient: 20-100% B (40 min); Elution: 19.9-21.7 minutes; Number of runs: 5.

### Example 2

### N-(1-(5-(3-cyano-6-ethoxypyrazolo[1,5-a]pyridin-4-yl)pyridin-2-yl)-4-((4-(7-(4-(2-(2,6-dioxopiperidin-3-yl)-1-oxoisoindolin-5-yl)piperazin-1-yl)heptanoyl)piperazin-1-yl)methyl)piperidin-4-yl)-3-fluoropicolinamide

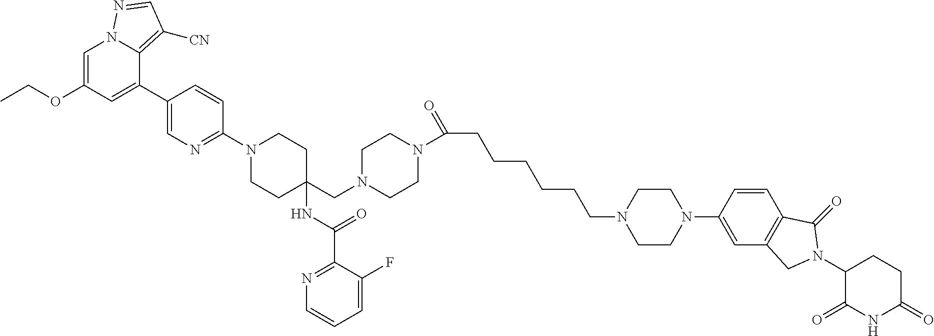 [SMILES: CCOc1cc(-c2ccc(N3CCC(CN4CCN(C(=O)CCCCCCN5CCN(c6ccc7c(c6)CN(C6CCC(=O)NC6=O)C7=O)CC5)CC4)(NC(=O)c4ncccc4F)CC3)nc2)c2c(C#N)cnn2c1]

### A. Tert-butyl oct-7-enoate

To a stirred solution of oct-7-enoic acid (1000 mg, 7.03 mmol) in DCM (20 mL) and tert-butanol (20 mL) was added DCC (1740 mg, 8.44 mmol) and DMAP (85 mg, 0.70 mmol), the mixture was stirred at rt overnight. The precipitate was filtered, and the filtrate was concentrated, then the residue was washed with Et₂O, and the filtrate was concentrated to afford the product (1.3 g, 93% yield) as a colorless oil, which was used directly in the next step without further purification.

### B. Tert-butyl 7-oxoheptanoate

To the mixture solution of tert-butyl oct-7-enoate (100 mg, 0.50 mmol) in tert-butanol (10 mL) and water (10 mL) was added potassium citrate (15 mg, 0.05 mmol), NMO (120 mg, 1.01 mmol), and citric acid (195 mg, 1.01 mmol), the resulting mixture was stirred at rt for 1 h, then reaction was cooled to 0 °C, sodium periodate (220 mg, 1.01 mmol) was added, the mixture was stirred for 0.5 h. The reaction mixture was diluted with water and extracted with ethyl acetate, the combined organic layers were washed with brine and dried over anhydrous sodium sulfate. After filtration, the filtrate was concentrated under reduced pressure. The resulting residue was purified by Prep TLC to afford tert-butyl 7-oxoheptanoate (80 mg, 79% yield) as a colorless oil. ¹H NMR (300 MHz, Chloroform-d) δ 9.77 (t, J=1.8 Hz, 1H), 2.44 (td, J=7.4, 1.8 Hz, 2H), 2.22 (t, J=7.4 Hz, 2H), 1.63 (ddt, J=15.1, 13.0, 7.4 Hz, 4H), 1.44 (s, 9H), 1.42-1.32 (m, 1H), 1.37-1.16 (m, 1H).

### C. Tert-butyl 7-(4-(2-(2,6-dioxopiperidin-3-yl)-1-oxoisoindolin-5-yl)piperazin-1-yl)heptanoate

To a stirred solution of 3-(1-oxo-5-piperazin-1-yl-isoindolin-2-yl)piperidine-2,6-dione (50 mg, 0.15 mmol) in DCM (5 mL) was added tert-butyl 7-oxoheptanoate (60 mg, 0.30 mmol), and DIEA (0.02 mL, 0.30 mmol). The mixture was stirred at rt for 5 min and then AcOH (0.02 mL, 0.30 mmol) was added, the mixture was stirred at rt for 0.5 h. The reaction was cooled to 0 °C, NaBH(OAc)₃ (65 mg, 0.30 mmol) was subsequently added, and the mixture was stirred at rt for 2 h. Upon completion, the reaction mixture was concentrated. The resulting residue was purified by Prep-HPLC to afford the desired product (50 mg, 64% yield) as an off-white semi solid. MS: m/z 513.2 [M+H]⁺.

### D. 7-(4-(2-(2,6-dioxopiperidin-3-yl)-1-oxoisoindolin-5-yl)piperazin-1-yl)heptanoic acid

To a stirred solution of tert-butyl 7-[4-[2-(2,6-dioxo-3-piperidyl)-1-oxo-isoindolin-5-yl]piperazin-1-yl]heptanoate (150 mg, 0.29 mmol) in DCM (10 mL) was added TFA (2 mL), the mixture solution was stirred at rt for 3 h. Upon completion, the reaction was concentrated to afford the desired product (120 mg, 86% yield) as an off whited semi solid. MS: m/z 457.3 [M+H]⁺.

### E. Methyl 1-(5-(3-cyano-6-ethoxypyrazolo[1,5-a]pyridin-4-yl)pyridin-2-yl)-4-(3-fluoropicolinamido)piperidine-4-carboxylate

To a mixture of methyl 4-amino-1-[5-(3-cyano-6-ethoxy-pyrazolo[1,5-a]pyridin-4-yl)-2-pyridyl]piperidine-4-carboxylate (1200 mg, 2.85 mmol) and 3-fluoropyridine-2-carboxylic acid (485 mg, 3.42 mmol) in DMF (20 mL) was added HATU (1300 mg, 3.42 mmol), DIEA (0.66 mL, 8.55 mmol), and the resulting mixture was stirred at rt for 2 h. The reaction was quenched by ice water, and extracted with ethyl acetate, and the combined organic extracts were washed with water and brine, dried over Na₂SO₄, and filtered. Concentration under reduced pressure followed by flash chromatography on silica gel afforded the desired product (1240 mg, 80% yield) as an off-white solid. MS: m/z 544 [M+H]⁺.

### F. N-(1-(5-(3-cyano-6-ethoxypyrazolo[1,5-a]pyridin-4-yl)pyridin-2-yl)-4-(hydroxymethyl)piperidin-4-yl)-3-fluoropicolinamide

To a solution of methyl 1-[5-(3-cyano-6-ethoxy-pyrazolo[1,5-a]pyridin-4-yl)-2-pyridyl]-4-[(3-fluoropyridine-2-carbonyl)amino]piperidine-4-carboxylate (1200 mg, 2.21 mmol) in THF (30 mL) and ethanol (30 mL) was added CaCl₂ (490 mg, 4.42 mmol) at rt. The resulting mixture was stirred at rt for 3 h, then the reaction mixture was cooled at 0 °C and NaBH₄ (167 mg, 4.42 mmol) was added. The mixture was stirred at ambient temperature (~15 °C) for overnight. Another batch of NaBH₄ (167 mg, 4.42 mmol) was added at 0 °C and the mixture was stirred at ambient temperature for another 8 h. A third batch of NaBH₄ (83.0 mg, 2.21 mmol) was added at 0 °C and the mixture was stirred for another 18 h. It was cooled to 0 °C in an ice/H₂O bath and quenched by dropwise addition of 10% citric acid. The resulting mixture was extracted with EtOAc, the combined organic layers were dried over sodium sulfate, concentrated in vacuo. The residue was purified by reversed phase column (eluting with 0.5% TFA in water/MeCN) to obtain the desired product (550 mg, 48.3% yield) as off-white semi-solid. MS: m/z 515.2 [M+H]⁺.

### G. N-(1-(5-(3-cyano-6-ethoxypyrazolo[1,5-a]pyridin-4-yl)pyridin-2-yl)-4-formylpiperidin-4-yl)-3-fluoropicolinamide

To a solution of N-[1-[5-(3-cyano-6-ethoxy-pyrazolo[1,5-a]pyridin-4-yl)-2-pyridyl]-4-(hydroxymethyl)-4-piperidyl]-3-fluoro-pyridine-2-carboxamide (400 mg, 0.78 mmol) in DCM (20 mL) was added Dess-Martin periodinane (410 mg, 1.16 mmol) at 0 °C. The reaction was stirred at rt for 3 h and then purified by Prep-TLC (elution with EA:DCM=2:1) to afford the desired product (220 mg, 55% yield) as an off-white solid. MS: m/z 514.1 [M+H]⁺.

### H. Tert-butyl 4-((1-(5-(3-cyano-6-ethoxypyrazolo[1,5-a]pyridin-4-yl)pyridin-2-yl)-4-(3-fluoropicolinamido)piperidin-4-yl)methyl)piperazine-1-carboxylate

To a stirred solution of N-[1-[5-(3-cyano-6-ethoxy-pyrazolo[1,5-a]pyridin-4-yl)-2-pyridyl]-4-formyl-4-piperidyl]-3-fluoro-pyridine-2-carboxamide (120 mg, 0.23 mmol) and tert-butyl piperazine-1-carboxylate (130 mg, 0.70 mmol) in DCM (10 ml) was added Ti(OiPr)₄ (133 mg, 0.47 mmol), and the resulting mixture was stirred at rt for 1 h. Then NaBH(OAc)₃ (99 mg, 0.47 mmol) was added at 0 °C and the mixture was stirred at rt overnight. The reaction was concentrated, and the residue was purified by Prep-TLC (EA:DCM=2:1) to afford the desired product (130 mg, 81% yield) as an off white solid. MS: m/z 684.5 [M+H]⁺.

### I. N-(1-(5-(3-cyano-6-ethoxypyrazolo[1,5-a]pyridin-4-yl)pyridin-2-yl)-4-(piperazin-1-ylmethyl)piperidin-4-yl)-3-fluoropicolinamide

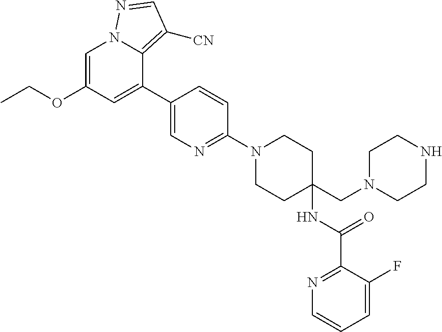 [SMILES: CCOc1cc(-c2ccc(N3CCC(CN4CCNCC4)(NC(=O)c4ncccc4F)CC3)nc2)c2c(C#N)cnn2c1]

To a mixture solution of tert-butyl 4-[[1-[5-(3-cyano-6-ethoxy-pyrazolo[1,5-a]pyridin-4-yl)-2-pyridyl]-4-[(3-fluoropyridine-2-carbonyl)amino]-4-piperidyl]methyl]piperazine-1-carboxylate (30 mg, 0.04 mmol) in DCM (5 mL) was added TFA (1 mL), the mixture solution was stirred at rt for 3 h, the reaction was concentrated and the residue was purified by Prep-HPLC to afford the desired product (11.8 mg, 0.02 mmol, 46.08% yield) as a white solid. ¹H NMR (400 MHz, DMSO-d6) δ 8.67 (d, J=2.1 Hz, 1H), 8.58 (s, 1H), 8.52-8.41 (m, 2H), 8.33 (d, J=2.5 Hz, 1H), 8.24 (brs, 1H), 7.89-7.79 (m, 2H), 7.67-7.62 (m, 1H), 7.65 (dt, J=8.5, 4.3 Hz, 1H), 7.04-7.02 (m, 1H), 4.21-4.13 (m, 4H), 3.19-3.07 (m, 6H), 2.84-2.75 (m 6), 2.39-2.35 (m, 2H), 1.71-1.64 (M, 2H), 1.38 (t, J=7.0 Hz, 3H); Analytical LCMS ESI-MS(+) m/z 584.5 [M+H]⁺, RT=1.31 min, purity: 97% (Method Q). Prep-HPLC purification condition: Column: Xselect CSH C18 OBD Column 30×150 mm 5 μm; Mobile Phase A: water (0.5% TFA), Mobile Phase B: ACN; Flow rate: 60 mL/min; Gradient: 8% B to 38% B in 10 min, 38% B; RT=7.62 min.

### J. N-(1-(5-(3-cyano-6-ethoxypyrazolo[1,5-a]pyridin-4-yl)pyridin-2-yl)-4-((4-(7-(4-(2-(2,6-dioxopiperidin-3-yl)-1-oxoisoindolin-5-yl)piperazin-1-yl)heptanoyl)piperazin-1-yl)methyl)piperidin-4-yl)-3-fluoropicolinamide

To a stirred solution of 7-[4-[2-(2,6-dioxo-3-piperidyl)-1-oxo-isoindolin-5-yl]piperazin-1-yl]heptanoic acid (20 mg, 0.04 mmol) in DMF (1 mL) were added N-(1-(5-(3-cyano-6-ethoxypyrazolo[1,5-a]pyridin-4-yl)pyridin-2-yl)-4-(piperazin-1-ylmethyl)piperidin-4-yl)-3-fluoropicolinamide (23.16 mg, 0.04 mmol), TCFH (24.99 mg, 0.07 mmol) and NMI (0.01 mL, 0.13 mmol). The mixture was stirred at rt for 2 h. The reaction was subjected to Prep-HPLC purification to afford the titled compound (21 mg, 48% yield) as an off-white solid. ¹H NMR (400 MHz, DMSO-d6) δ 10.96 (s, 1H), 9.88 (s, 1H), 8.70-8.57 (m, 2H), 8.53-8.50 (m, 1H), 8.36 (d, J=2.6 Hz, 1H), 7.94-7.87 (m, 1H), 7.83 (dd, J=8.8, 2.6 Hz, 1H), 7.73-7.67 (m, 1H), 7.60 (d, J=8.4 Hz, 1H), 7.30-7.26 (m, 1H), 7.21-7.12 (m, 2H), 7.08 (d, J=8.8 Hz, 1H), 5.07 (dd, J=13.3, 5.1 Hz, 1H), 4.39-4.20 (m, 6H), 4.19-4.14 (m, 3H), 3.80-3.47 (m, 6H), 3.25-3.07 (m, 10H), 2.98-2.84 (m, 1H), 2.64-2.52 (m, 5H), 2.46-2.39 (m, 1H), 2.38-2.30 (m, 3H), 2.03-1.92 (m, 1H), 1.82-1.64 (m, 4H), 1.57-1.45 (m, 2H), 1.43-1.27 (m, 7H); Analytical LCMS ESI-MS(+) m/z 1022.6 [M+H]⁺, RT=1.32 min, purity: 97% (Method Q). Prep-HPLC purification condition: Column: Xselect CSH C18 OBD Column 30×150 mm 5 μm; Mobile Phase A: water (0.5% TFA), Mobile Phase B: ACN; Flow rate: 60 mL/min; Gradient: 12% B to 22% B in 12 min, 22% B; RT=17.05 min.

### Example 3

### N-(1-(5-(3-cyano-6-ethoxypyrazolo[1,5-a]pyridin-4-yl)pyridin-2-yl)-4-((4-(7-(4-(2-(2,6-dioxopiperidin-3-yl)-1-oxoisoindolin-5-yl)piperazin-1-yl)heptanoyl)piperazin-1-yl)methyl)piperidin-4-yl)-3-methylbutanamide

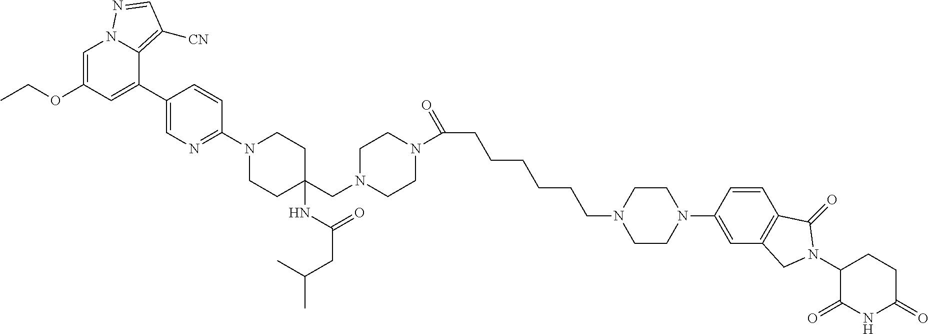 [SMILES: CCOc1cc(-c2ccc(N3CCC(CN4CCN(C(=O)CCCCCCN5CCN(c6ccc7c(c6)CN(C6CCC(=O)NC6=O)C7=O)CC5)CC4)(NC(=O)CC(C)C)CC3)nc2)c2c(C#N)cnn2c1]

The title compound was prepared in 61% yield as a white solid according to the preparation of Example 2 using N-(1-(5-(3-cyano-6-ethoxypyrazolo[1,5-a]pyridin-4-yl)pyridin-2-yl)-4-(piperazin-1-ylmethyl)piperidin-4-yl)-3-methylbutanamide in STEP E. 1H NMR (300 MHz, DMSO-d6) δ 10.96 (s, 1H), 9.86 (s, 1H), 8.67 (d, J=2.1 Hz, 1H), 8.58 (s, 1H), 8.35 (d, J=2.5 Hz, 1H), 8.02 (s, 1H), 7.83 (dd, J=9.0, 2.5 Hz, 1H), 7.60 (d, J=8.3 Hz, 1H), 7.28 (d, J=2.1 Hz, 1H), 7.21-7.13 (m, 2H), 7.06 (d, J=9.0 Hz, 1H), 5.10-5.01 (m, 1H), 4.44-4.32 (m, 2H), 4.30-4.10 (m, 6H), 4.08-3.96 (m, 3H), 3.65-3.41 (m, 7H), 3.21-3.06 (m, 12H), 2.65-2.55 (m, 1H), 2.40-2.27 (m, 4H), 2.18-2.10 (m, 2H), 2.06-1.94 (m, 2H), 1.74-1.60 (m, 4H), 1.56-1.45 (m, 2H), 1.43-1.27 (m, 7H), 0.91 (d, J=6.4 Hz, 6H); Analytical LCMS ESI-MS(+) m/z 983.5 [M+H]⁺, RT=1.71 min, purity: 99% (Method P); Prep-HPLC purification condition: Column: Xselect CSH C18 OBD Column 30×150 mm 5 μm; Mobile Phase A: water (0.5% TFA), Mobile Phase B: ACN; Flow rate: 60 mL/min; Gradient: 17% B to 30% B in 13 min, 30% B; RT=12.65 min.

### Example 4

### N-(1-(5-(3-cyano-6-ethoxypyrazolo[1,5-a]pyridin-4-yl)pyridin-2-yl)-4-((4-(3-(4-(2-(2,6 dioxopiperidin-3-yl)-3-oxoisoindolin-5-yl)piperidin-1-yl)propanoyl)piperazin-1-yl)methyl)piperidin-4-yl)-3-fluoropicolinamide

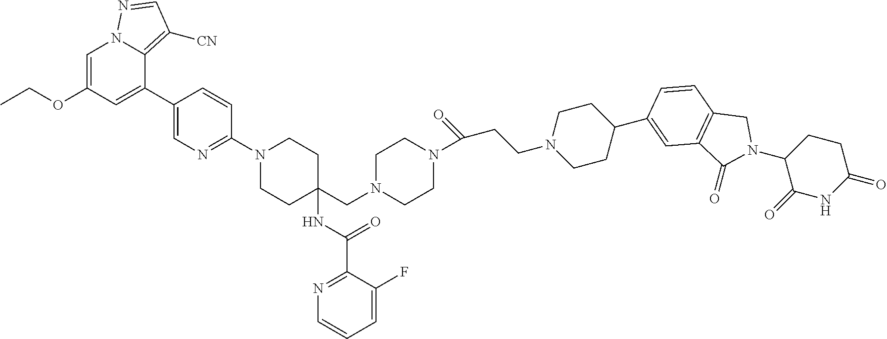 [SMILES: CCOc1cc(-c2ccc(N3CCC(CN4CCN(C(=O)CCN5CCC(c6ccc7c(c6)C(=O)N(C6CCC(=O)NC6=O)C7)CC5)CC4)(NC(=O)c4ncccc4F)CC3)nc2)c2c(C#N)cnn2c1]

### A. Tert-butyl 3-(4-(2-(2,6-dioxopiperidin-3-yl)-3-oxoisoindolin-5-yl)piperidin-1-yl)propanoate

To a stirred solution of 3-[1-oxo-6-(4-piperidyl) isoindolin-2-yl]piperidine-2,6-dione (50 mg, 0.15 mmol) in CH₃CN (5 mL) were added tert-butyl prop-2-enoate (40 mg, 0.31 mmol), DBU (0.05 mL, 0.31 mmol) and the reaction mixture was stirred at rt for overnight. The reaction was concentrated and purified by reverse-phase column chromatography to afford the desired product (60 mg, 86% yield) as an off-white semi solid. MS: m/z 456.4 [M+H]⁺.

### B. 3-(4-(2-(2,6-Dioxopiperidin-3-yl)-3-oxoisoindolin-5-yl)piperidin-1-yl)propanoic acid

To a stirred solution of tert-butyl 3-[4-[2-(2,6-dioxo-3-piperidyl)-3-oxo-isoindolin-5-yl]-1-piperidyl]propanoate (60 mg, 0.13 mmol) in DCM (10 mL) was added TFA (0.02 mL), the mixture solution was stirred at rt for 3 h. The reaction was concentrated to afford the desired product (50 mg, 91% yield) as an off-white semi solid. MS: m/z 400.3 [M+H]⁺.

### C. N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)propanoyl)piperazin-1-yl)methyl)piperidin-4-yl)-3-fluoropicolinamide

To a stirred solution of 3-[4-[2-(2,6-dioxo-3-piperidyl)-3-oxo-isoindolin-5-yl]-1-piperidyl]propanoic acid (30 mg, 0.08 mmol) in DMF (3 mL) was added N-[1-[5-(3-cyano-6-ethoxy-pyrazolo[1,5-a]pyridin-4-yl)-2-pyridyl]-4-(piperazin-1-ylmethyl)-4-piperidyl]-3-fluoro-pyridine-2-carboxamide (44 mg, 0.08 mmol), HATU (60 mg, 0.15 mmol) and DIEA (0.04 mL, 0.23 mmol), the mixture was stirred at 0 °C for 2 h. The reaction was purified by reverse-phase column chromatography to give the crude, and further purified by Prep-HPLC to afford the titled compound (25.8 mg, 35% yield) as an off-white solid. ¹H NMR (400 MHz, DMSO-d6) δ 10.99 (s, 1H), 9.80 (brs, 1H), 8.68-8.58 (m, 2H), 8.52-8.50 (m, 1H), 8.46 (s, 1H), 8.36 (d, J=2.6 Hz, 1H), 7.95-7.86 (m, 1H), 7.73-7.67 (m, 1H), 7.62-7.56 (m, 2H), 7.55-7.51 (m, 1H), 7.31-7.24 (m, 1H), 7.13-7.00 (m, 1H), 5.13-5.07 (m, 1H), 4.49-4.40 (m, 2H), 4.35-4.24 (m, 4H), 4.16-4.12 (m, 2H), 3.70-3.51 (m, 6H), 3.43-3.26 (m, 4H), 3.23-3.05 (m, 5H), 3.01-3.83 (m, 5H), 2.65-2.57 (m, 2H), 2.48-2.33 (m, 2H), 2.10-1.89 (m, 5H), 1.84-1.70 (m, 2H), 1.39 (t, J=7.0 Hz, 3H); Analytical LCMS ESI-MS(+) m/z 965.6 [M+H]⁺, RT=1.33 min, purity: 98% (Method Q); Prep-HPLC purification condition: Column: Xselect CSH C18 OBD Column 30×150 mm 5 μm; Mobile Phase A: water (0.5% TFA), Mobile Phase B: ACN; Flow rate: 60 mL/min; Gradient: 12% B to 22% B in 12 min, 22% B; RT=17.05 min.

### Example 5

### N-(1-(5-(3-cyano-6-ethoxypyrazolo[1,5-a]pyridin-4-yl)pyridin-2-yl)-4-((4-(5-(4-(2-(2,6-dioxopiperidin-3-yl)-1-oxoisoindolin-5-yl)piperazin-1-yl)pentanoyl)piperazin-1-yl)methyl)piperidin-4-yl)-2,5-difluorobenzamide

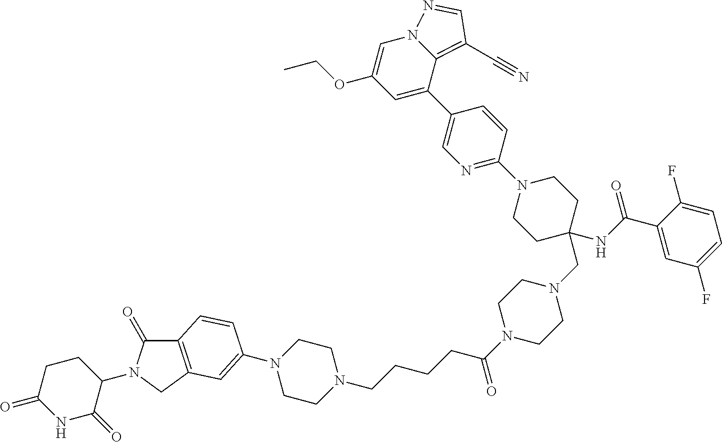 [SMILES: CCOc1cc(-c2ccc(N3CCC(CN4CCN(C(=O)CCCCN5CCN(c6ccc7c(c6)CN(C6CCC(=O)NC6=O)C7=O)CC5)CC4)(NC(=O)c4cc(F)ccc4F)CC3)nc2)c2c(C#N)cnn2c1]

### A. Tert-butyl 5-(methoxy(methyl)amino)-5-oxopentanoate

To a stirred solution of 5-tertbutoxy-5-oxo-pentanoic acid (1.0 g, 5.31 mmol) in DCM (20 mL) were added N-methoxymethanamine hydrogen hydrochloride (1.0 g, 10.63 mmol), HATU (3.0 g, 7.97 mmol), and DIEA (1.63 mL, 21.25 mmol) at −5 °C. The reaction mixture was stirred at rt for 3 h, concentrated and purified by silica gel column chromatography (elution with PE:EA=10:1) to afford the desired product (1.1 g, 89.5% yield) as a colorless oil. MS: m/z 232 [M+H]⁺.

### B. Tert-butyl 5-oxopentanoate

To a stirred solution of tert-butyl 5-[methoxy(methyl)amino]-5-oxo-pentanoate (800 mg, 3.46 mmol) in THF (10 mL) was added a solution of LiAlH₄ (1 mol/L in THF, 6 mL, 6 mmol) dropwise at 0 °C, and the mixture solution was stirred for 3 min. The reaction was quenched by slowly adding a saturated aqueous solution of sodium sulfate dropwise, the resulting mixture was filtered through Celite, and the filter cake washed thoroughly with ethyl acetate. The filtrate was concentrated under reduced pressure. Purification using flash chromatography (elution with PE:EA=3:1) to afford tert-butyl 5-oxopentanoate (300 mg, 50.4% yield) as a colorless oil. ¹H NMR (300 MHz, Chloroform-d) δ 9.78 (t, J=1.5 Hz, 1H), 2.63-2.43 (m, 2H), 2.30-2.25 (m, 2H), 1.96-1.86 (m, 2H), 1.45 (s, 9H).

### C. Tert-butyl 5-(4-(2-(2,6-dioxopiperidin-3-yl)-1-oxoisoindolin-5-yl)piperazin-1-yl)pentanoate

To a stirred solution of 3-(1-oxo-5-piperazin-1-yl-isoindolin-2-yl)piperidine-2,6-dione (100 mg, 0.30 mmol) in DCM (1 mL) was added tert-butyl 5-oxopentanoate (52.45 mg, 0.30 mmol). The mixture was stirred at rt for 1 h. NaBH(OAc)₃ (129.09 mg, 0.61 mmol) was added at 0 °C and was allowed to stir at rt for 2 h. The reaction mixture was concentrated, and the resulting crude product was purified by reverse-phase column chromatography to afford the desired product (110 mg, 74.5% yield) as a white solid. MS: m/z 485 [M+H]⁺.

### D. 5-(4-(2-(2,6-Dioxopiperidin-3-yl)-1-oxoisoindolin-5-yl)piperazin-1-yl)pentanoic acid

To a stirred solution of tert-butyl 5-[4-[2-(2,6-dioxo-3-piperidyl)-1-oxo-isoindolin-5-yl]piperazin-1-yl]pentanoate (100 mg, 0.21 mmol) in DCM (3 mL) was added TFA (1 mL). The mixture was stirred at rt for 2 h and then concentrated to afford crude product, which was used directly in the next step without further purification. MS: [M+H]⁺ m/z 429.

### E. N-(1-(5-(3-cyano-6-ethoxypyrazolo[1,5-a]pyridin-4-yl)pyridin-2-yl)-4-((4-(5-(4-(2-(2,6-dioxopiperidin-3-yl)-1-oxoisoindolin-5-yl)piperazin-1-yl)pentanoyl)piperazin-1-yl)methyl)piperidin-4-yl)-2,5-difluorobenzamide

To a stirred solution of 5-[4-[2-(2,6-dioxo-3-piperidyl)-1-oxo-isoindolin-5-yl]piperazin-1-yl]pentanoic acid (30 mg, 0.07 mmol) in DMF (3 mL) were added N-[1-[5-(3-cyano-6-ethoxy-pyrazolo[1,5-a]pyridin-4-yl)-2-pyridyl]-4-(piperazin-1-ylmethyl)-4-piperidyl]-2,5-difluoro-benzamide (43 mg, 0.07 mmol), HATU (40 mg, 0.11 mmol), DIEA (24 mg, 0.22 mmol), and the mixture was stirred at rt for 2 h. The reaction was purified by reverse-phase column chromatography to afford the crude, and further purified by Prep-HPLC to give the titled compound (27.8 mg, 38.1% yield) as an off-white solid. ¹H NMR (300 MHz, DMSO-d6) δ 10.97 (s, 1H), 9.67 (brs, 1H), 8.68 (d, J=2.1 Hz, 1H), 8.59 (s, 1H), 8.36-8.35 (m, 1H), 7.83-7.80 (m, 1H), 7.62-7.59 (m, 1H), 7.53-7.37 (m, 3H), 7.28 (d, J=2.1 Hz, 1H), 7.21-7.12 (m, 2H), 7.09-7.03 (m, 1H), 5.10-5.04 (m, 1H), 4.42-4.10 (m, 11H), 4.09-3.99 (m, 3H), 3.64-3.46 (m, 5H), 3.28-3.07 (m, 9H), 3.01-2.83 (m, 2H), 2.65-2.56 (m, 2H), 2.46-2.35 (s, 4H), 2.02-1.92 (m, 1H), 1.76-1.64 (m, 3H), 1.60-1.51 (m, 2H), 1.39 (t, J=6.9 Hz, 3H); Analytical LCMS ESI-MS(+) m/z 1011.6 [M+H]⁺, RT=1.43 min, purity: 97% (Method Q); Prep-HPLC purification conditions: Column: Xselect CSH C18 OBD Column 30*150 mm 5 μm; Mobile Phase A: water (0.5% TFA), Mobile Phase B: ACN; Flow rate: 60 mL/min; Gradient: 3% B to 33% B in 10 min, 33% B; RT=10.8 min.

### Example 6

### N-(1-(5-(3-cyano-6-ethoxypyrazolo[1,5-a]pyridin-4-yl)pyridin-2-yl)-4-((4-(2-(4-(2-(2,6-dioxopiperidin-3-yl)-1-oxoisoindolin-5-yl)piperazin-1-yl)acetyl)piperazin-1-yl)methyl)piperidin-4-yl)-2,5-difluorobenzamide

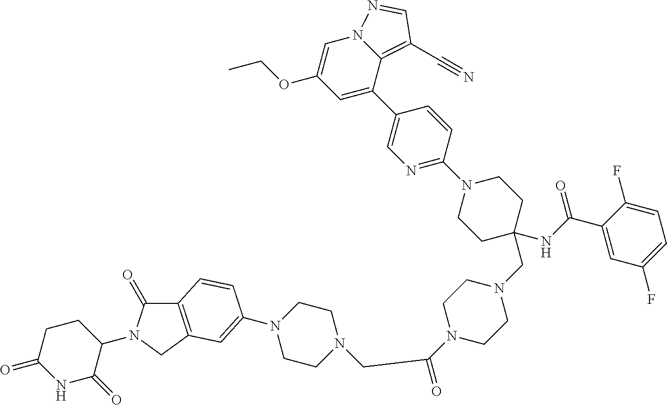 [SMILES: CCOc1cc(-c2ccc(N3CCC(CN4CCN(C(=O)CN5CCN(c6ccc7c(c6)CN(C6CCC(=O)NC6=O)C7=O)CC5)CC4)(NC(=O)c4cc(F)ccc4F)CC3)nc2)c2c(C#N)cnn2c1]

### A. Tert-butyl 2-oxoacetate

To a stirred solution of tert-butyl prop-2-enoate (1000 mg, 7.8 mmol) in water (30 mL) and tert-butanol (30 mL) was added potassium citrate (240 mg, 0.78 mmol), Citric acid (3000 mg, 0.78 mmol), and NMO (1830 mg, 15.6 mmol). The mixture was stirred at rt for 1 h, then sodium periodate (2503 mg, 11.7 mmol) was added, and the mixture was stirred at 0 °C for 0.5 h. The reaction mixture was diluted with ethyl acetate, the biphasic mixture was partitioned in a separatory funnel. The aqueous layer was extracted twice with EtOAc, and the combined organic extracts were washed with water and brine, dried over Na₂SO₄, and filtered. Concentration under reduced pressure followed by Prep-TLC afforded tert-butyl 2-oxoacetate (340 mg, 33.5% yield) as a light-yellow oil.

### B. Tert-butyl 2-(4-(2-(2,6-dioxopiperidin-3-yl)-3-oxoisoindolin-5-yl)piperazin-1-yl)acetate

To a stirred solution of 3-(1-oxo-5-piperazin-1-yl-isoindolin-2-yl)piperidine-2,6-dione (70 mg, 0.21 mmol) in DCM (5 mL) were added tert-butyl 2-oxoacetate (55 mg, 0.43 mmol), and DIEA (0.04 mL, 0.43 mmol). It was stirred at rt for 5 min, and acetic acid (0.02 mL, 0.43 mmol) was added. The reaction was stirred at rt for 0.5 h, NaBH(OAc)₃ (90 mg, 0.43 mmol) was then added at 0 °C. It was stirred at rt for 4 h, concentrated and purified by flash chromatograph to afford the desired product (80 mg, 81.5% yield) as an off-white semi-solid. MS: [M+H]⁺ m/z 443.7.

### C. 2-(4-(2-(2,6-dioxopiperidin-3-yl)-3-oxoisoindolin-5-yl)piperazin-1-yl)acetic acid

To a stirred solution of tert-butyl 2-[4-[2-(2,6-dioxo-3-piperidyl)-3-oxo-isoindolin-5-yl] piperazin-1-yl]acetate (80 mg, 0.18 mmol) in DCM (10 mL) was added TFA (0.03 mL). The reaction mixture was stirred at rt for 4 h. The reaction was concentrated to afford 2-[4-[2-(2,6-dioxo-3-piperidyl)-3-oxo-isoindolin-5-yl]piperazin-1-yl] acetic acid (65 mg, 89.4% yield), which was used for the next step without further purification. MS: m/z [M+H]⁺ 387.3.

### D. N-(1-(5-(3-cyano-6-ethoxypyrazolo[1,5-a]pyridin-4-yl)pyridin-2-yl)-4-((4-(2-(4-(2-(2,6-dioxopiperidin-3-yl)-1-oxoisoindolin-5-yl)piperazin-1-yl)acetyl)piperazin-1-yl)methyl)piperidin-4-yl)-2,5-difluorobenzamide

To a stirred solution of 2-[4-[2-(2,6-dioxo-3-piperidyl)-3-oxo-isoindolin-5-yl]piperazin-1-yl]acetic acid (30 mg, 0.08 mmol) in DMF (3 mL) was added N-[1-[5-(3-cyano-6-ethoxy-pyrazolo[1,5-a]pyridin-4-yl)-2-pyridyl]-4-(piperazin-1-ylmethyl)-4-piperidyl]-2,5-difluoro-benzamide (48 mg, 0.08 mmol), HATU (45 mg, 0.12 mmol), the mixture was cooled down to 0 °C, then DIEA (0.02 mL, 0.23 mmol) was added, the mixture was stirred at 0 °C for 2 h. The reaction was purified by pre-HPLC to afford the desired product (11.9 mg, 15.6% yield) as a white solid. ¹H NMR (400 MHz, DMSO-d6) δ 10.98 (s, 1H), 10.18 (brs, 1H), 8.68 (d, J=2.0 Hz, 1H), 8.59 (s, 1H), 8.36 (d, J=2.6 Hz, 1H), 7.84 (dd, J=8.9, 2.6 Hz, 1H), 7.61 (d, J=8.5 Hz, 1H), 7.51-7.33 (m, 3H), 7.29 (d, J=2.0 Hz, 1H), 7.20-7.11 (m, 2H), 7.08 (d, J=8.9 Hz, 1H), 5.07 (dd, J=13.3, 5.1 Hz, 1H), 4.46-4.33 (m, 3H), 4.30-4.13 (m, 5H), 4.02-3.89 (m, 2H), 3.67-3.06 (m, 15H), 2.98-2.85 (m, 2H), 2.64-2.55 (m, 3H), 2.40-2.35 (m, 3H), 2.41 (s, 1H), 1.79-1.64 (m, 2H), 1.39 (t, J=6.9 Hz, 3H); Analytical LCMS ESI-MS(+) m/z 969.3 [M+H]⁺, RT=1.63 min, purity: 99% (Method Q); Prep-HPLC purification conditions: Column: Xselect CSH C18 OBD Column 30×150 mm 5 μm; Mobile Phase A: water (0.5% TFA), Mobile Phase B: ACN; Flow rate: 60 mL/min; Gradient: 9% B to 33% B in 10 min, 33% B; RT=10.25 min.

### Example 7

### N-(1-(5-(3-cyano-6-ethoxypyrazolo[1,5-a]pyridin-4-yl)pyridin-2-yl)-4-((4-(7-(4-(2-(2,6-dioxopiperidin-3-yl)-1-oxoisoindolin-5-yl)piperazin-1-yl)heptanoyl)piperazin-1-yl)methyl)piperidin-4-yl)cyclopropanecarboxamide

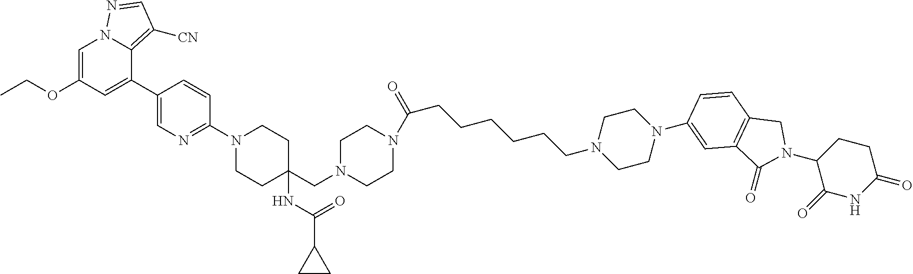 [SMILES: CCOc1cc(-c2ccc(N3CCC(CN4CCN(C(=O)CCCCCCN5CCN(c6ccc7c(c6)C(=O)N(C6CCC(=O)NC6=O)C7)CC5)CC4)(NC(=O)C4CC4)CC3)nc2)c2c(C#N)cnn2c1]

To a stirred solution of 7-[4-[2-(2,6-dioxo-3-piperidyl)-1-oxo-isoindolin-5-yl]piperazin-1-yl]heptanoic acid (20 mg, 0.04 mmol) in DMF (1 mL) were added N-[1-[5-(3-cyano-6-ethoxy-pyrazolo[1,5-a]pyridin-4-yl)-2-pyridyl]-4-(piperazin-1-ylmethyl)-4-piperidyl]cyclopropanecarboxamide (23.16 mg, 0.04 mmol), TCFH (24.99 mg, 0.07 mmol) and NMI (0.01 mL, 0.13 mmol). The mixture was stirred at rt for 2 h, and the reaction was directly subjected to Prep-HPLC purification to afford the titled compound (10.5 mg, 0.01 mmol, 24% yield) as an off-white solid. ¹H NMR (400 MHz, DMSO-d6) δ 10.96 (s, 1H), 8.73-8.53 (m, 2H), 8.35-8.14 (m, 1H), 7.83-7.70 (m, 2H), 7.56-7.53 (m, 1H), 7.28-7.26 (m, 1H), 7.11-7.08 (m, 2H), 6.97-6.94 (m, 1H), 5.06 (dd, J=13.4, 5.3 Hz, 1H), 4.40-4.08 (m, 6H), 3.54-3.28 (m, 10H), 3.13-3.06 (m, 2H), 2.95-2.86 (m, 2H), 2.68-2.60 (m, 5H), 2.47-2.40 (m, 5H), 2.32-2.16 (m, 4H), 2.02-1.70 (m, 3H), 1.64-1.46 (m, 6H), 1.40-1.29 (m, 7H), 0.64-0.59 (m, 4H); Analytical LCMS ESI-MS(+) m/z 968 [M+H]⁺, RT=1.18 min, purity: 99.8% (Method Q); Prep-HPLC purification condition: Column: Xselect CSH C18 OBD Column 30×150 mm 5 μm; Mobile Phase A: Water (0.1% FA), Mobile Phase B: ACN; Flow rate: 60 mL/min; Gradient: 2% B to 27% B in 10 min, 27% B; RT=9.85 min.

### Example 8

### N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)propanoyl)piperazin-1-yl)methyl)piperidin-4-yl)-3-methylbutanamide

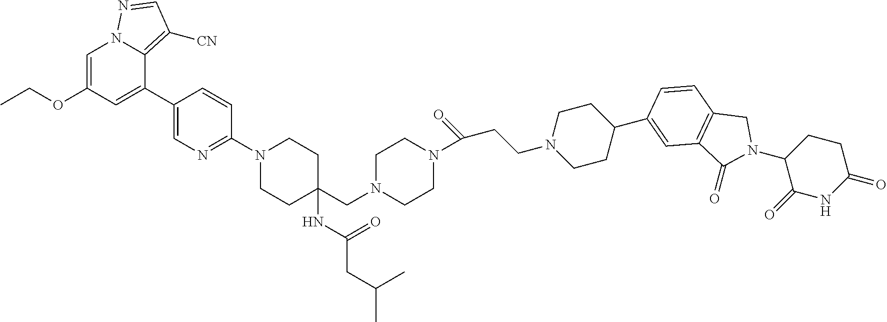 [SMILES: CCOc1cc(-c2ccc(N3CCC(CN4CCN(C(=O)CCN5CCC(c6ccc7c(c6)C(=O)N(C6CCC(=O)NC6=O)C7)CC5)CC4)(NC(=O)CC(C)C)CC3)nc2)c2c(C#N)cnn2c1]

The title compound was prepared in 18% yield as a white solid according to the preparation of Example 4 using N-(1-(5-(3-cyano-6-ethoxypyrazolo[1,5-a]pyridin-4-yl)pyridin-2-yl)-4-(piperazin-1-ylmethyl)piperidin-4-yl)-3-methylbutanamide in STEP C. ¹H NMR (400 MHz, DMSO-d6) δ 10.98 (s, 1H), 8.64 (d, J=2.1 Hz, 1H), 8.57 (s, 1H), 8.31 (d, J=2.6 Hz, 1H), 8.18 (s, 1H), 7.74 (dd, J=8.9, 2.6 Hz, 1H), 7.56-7.52 (m, 3H), 7.40 (s, 1H), 7.26 (d, J=2.1 Hz, 1H), 6.95 (d, J=8.9 Hz, 1H), 5.13-5.08 (m, 1H), 4.43-4.39 (m, 1H), 4.30-4.26 (m, 1H), 4.20-4.10 (m, 4H), 3.45-3.41 (m, 7H), 3.11-2.98 (m, 5H), 2.98-2.85 (m, 1H), 2.71-2.56 (m, 6H), 2.44-2.34 (m, 3H), 2.28-2.06 (m, 4H), 2.05-1.92 (m, 4H), 1.84-1.76 (m, 2H), 1.73-1.61 (m, 2H), 1.59-1.49 (m, 2H), 1.38 (t, J=6.9 Hz, 3H), 0.89 (d, J=6.1 Hz, 6H); Analytical LCMS ESI-MS(+) m/z 926.6 [M+H]⁺, RT=1.41 min, purity: 96.9% (Method Q); Prep-HPLC purification condition: Column: Xselect CSH C18 OBD Column 30×150 mm 5 μm; Mobile Phase A: Water (0.1% FA), Mobile Phase B: ACN; Flow rate: 60 mL/min; Gradient: 2% B to 27% B in 10 min, 27% B; RT=9.87 min.

### Example 9

### N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)propanoyl)piperazin-1-yl)methyl)piperidin-4-yl)cyclopropanecarboxamide

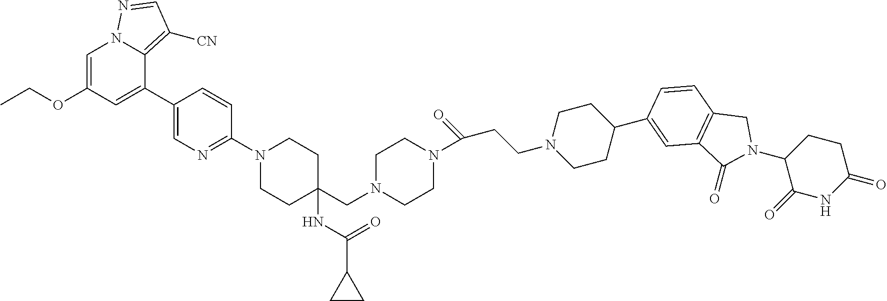 [SMILES: CCOc1cc(-c2ccc(N3CCC(CN4CCN(C(=O)CCN5CCC(c6ccc7c(c6)C(=O)N(C6CCC(=O)NC6=O)C7)CC5)CC4)(NC(=O)C4CC4)CC3)nc2)c2c(C#N)cnn2c1]

The title compound was prepared in 15% yield as a white solid according to the preparation of Example 4 (using N-(1-(5-(3-cyano-6-ethoxypyrazolo[1,5-a]pyridin-4-yl)pyridin-2-yl)-4-(piperazin-1-ylmethyl)piperidin-4-yl)cyclopropanecarboxamide in STEP C. ¹H NMR (400 MHz, DMSO-d6) δ 8.63-8.61 (m, 1H), 8.57-8.55 (m, 1H), 8.31 (d, J=2.5 Hz, 1H), 8.23 (s, 2H), 7.75 (dd, J=8.8, 2.5 Hz, 1H), 7.60-7.58 (m, 1H), 7.56-7.53 (m, 1H), 7.26-7.24 (m, 1H), 6.96 (d, J=8.8 Hz, 1H), 5.09 (dd, J=13.3, 5.1 Hz, 1H), 4.46-4.41 (m, 1H), 4.33-4.28 (m, 1H), 4.17-4.10 (m, 4H), 3.62-3.59 (m, 2H), 3.51-3.45 (m, 4H), 3.29-3.26 (m, 2H), 3.13-3.06 (m, 2H), 2.95-2.86 (m, 3H), 2.84-2.75 (m, 1H), 2.71-2.58 (m, 6H), 2.47-2.36 (m, 4H), 2.25-2.22 (m, 2H), 2.09-1.95 (m, 1H), 1.93-1.69 (m, 4H), 1.60-1.53 (m, 2H), 1.39 (t, J=6.9 Hz, 3H), 0.83-0.32 (m, 4H); Analytical LCMS ESI-MS(+) m/z 910 [M+H]⁺, RT=1.15 min, purity: 99.3% (Method Q); Prep-HPLC purification condition: Column: Xselect CSH C18 OBD Column 30×150 mm, 5 μm; Mobile Phase A: Water (0.1% FA), Mobile Phase B: ACN; Flow rate: 60 mL/min; Gradient: 2% B to 25% B in 10 min, 25% B; RT=10.15 min.

### Example 10

### N-(1-(5-(3-cyano-6-ethoxypyrazolo[1,5-a]pyridin-4-yl)pyridin-2-yl)-4-((4-(5-(4-(2-(2,6-dioxopiperidin-3-yl)-1-oxoisoindolin-5-yl)piperidin-1-yl)pentanoyl)piperazin-1-yl)methyl)piperidin-4-yl)-2,5-difluorobenzamide

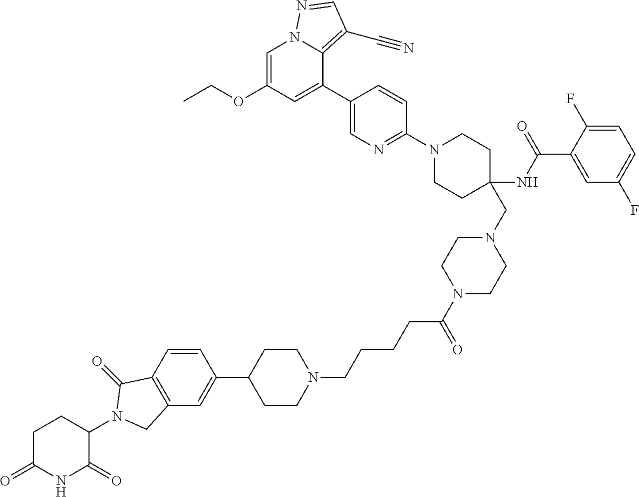 [SMILES: CCOc1cc(-c2ccc(N3CCC(CN4CCN(C(=O)CCCCN5CCC(c6ccc7c(c6)CN(C6CCC(=O)NC6=O)C7=O)CC5)CC4)(NC(=O)c4cc(F)ccc4F)CC3)nc2)c2c(C#N)cnn2c1]

The title compound was prepared in 30% overall yield as a white solid according to the preparation of Example 5 using 3-(1-oxo-5-(piperazin-1-yl) isoindolin-2-yl)piperidine-2,6-dione in STEP C. ¹H NMR (300 MHz, DMSO-d6) δ 8.68 (d, J=2.0 Hz, 1H), 8.59-8.58 (m, 1H), 8.37 (d, J=2.5 Hz, 1H), 7.85 (dd, J=8.9, 2.5 Hz, 1H), 7.72 (d, J=7.9 Hz, 1H), 7.57-7.34 (m, 5H), 7.29 (d, J=2.0 Hz, 1H), 7.09 (d, J=8.9 Hz, 1H), 5.12 (dd, J=13.2, 5.1 Hz, 1H), 4.49-4.43 (m, 1H), 4.38-4.23 (m, 3H), 4.16-4.12 (m, 2H), 3.69-3.55 (m, 4H), 3.51-3.49 (m, 4H), 3.38-2.84 (m, 13H), 2.67-2.55 (m, 1H), 2.48-2.32 (m, 4H), 2.13-1.84 (m, 5H), 1.81-1.48 (m, 6H), 1.39 (t, J=6.9 Hz, 3H). MS: m/z: [M+H]⁺ 1010.6. Prep-HPLC purification conditions: Column: Xselect CSH C18 OBD Column 30×150 mm 5 μm; Mobile Phase A: Water (0.5% TFA), Mobile Phase B: ACN; Flow rate: 60 mL/min; Gradient: 6% B to 36% B in 10 min, 36% B; RT=9.67 min.

### Example 11

### N-(1-(5-(3-cyano-6-ethoxypyrazolo[1,5-a]pyridin-4-yl)pyridin-2-yl)-4-((4-(7-(4-(2-(2,6-dioxopiperidin-3-yl)-1-oxoisoindolin-5-yl)piperidin-1-yl)heptanoyl)piperazin-1-yl)methyl)piperidin-4-yl)-2,5-difluorobenzamide

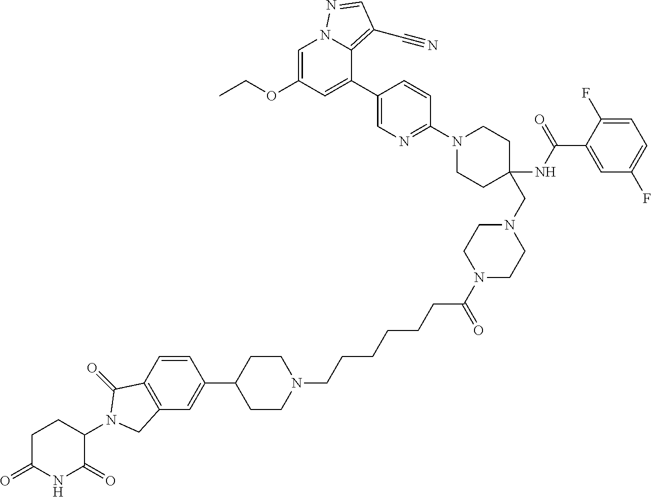 [SMILES: CCOc1cc(-c2ccc(N3CCC(CN4CCN(C(=O)CCCCCCN5CCC(c6ccc7c(c6)CN(C6CCC(=O)NC6=O)C7=O)CC5)CC4)(NC(=O)c4cc(F)ccc4F)CC3)nc2)c2c(C#N)cnn2c1]

The title compound was prepared in 17% overall yield as a white solid according to the preparation of Example 5 using tert-butyl 7-oxoheptanoate and 3-(1-oxo-5-(piperidin-4-yl) isoindolin-2-yl)piperidine-2,6-dione in STEP C. ¹H NMR (300 MHz, DMSO-d6) δ 8.68 (d, J=2.1 Hz, 1H), 8.59-8.56 (m, 1H), 8.36 (d, J=2.5 Hz, 1H), 7.85 (dd, J=8.9, 2.5 Hz, 1H), 7.72 (d, J=7.9 Hz, 1H), 7.56-7.37 (m, 5H), 7.29 (d, J=2.1 Hz, 1H), 7.09 (d, J=8.9 Hz, 1H), 5.14-5.09 (m, 1H), 4.49-4.27 (m, 3H), 4.16-4.12 (m, 3H), 3.69-3.61 (m, 4H), 3.59-3.54 (m, 7H), 3.30-2.84 (m, 10H), 2.67-2.57 (m, 1H), 2.48-2.31 (m, 4H), 2.12-2.85 (m, 5H), 1.81-1.64 (m, 4H), 1.59-1.47 (m, 2H), 1.41-1.33 (m, 7H); MS: m/z [M+H]⁺ 1038.7, 98% purity. Prep-HPLC purification conditions: Column: Xselect CSH C18 OBD Column 30×150 mm 5 μm; Mobile Phase A: water (0.5% TFA), Mobile Phase B: ACN; Flow rate: 60 mL/min; Gradient: 2% B to 32% B in 10 min, 32% B; RT=12.63 min.

### Example 12

### N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperazin-1-yl)propanoyl)piperazin-1-yl)methyl)piperidin-4-yl)-2,5-difluorobenzamide

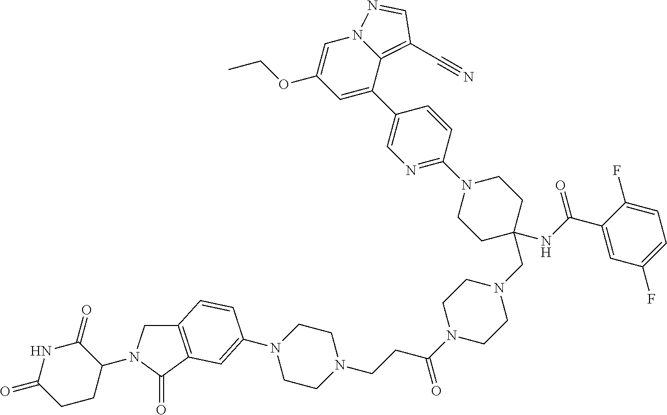 [SMILES: CCOc1cc(-c2ccc(N3CCC(CN4CCN(C(=O)CCN5CCN(c6ccc7c(c6)C(=O)N(C6CCC(=O)NC6=O)C7)CC5)CC4)(NC(=O)c4cc(F)ccc4F)CC3)nc2)c2c(C#N)cnn2c1]

To a solution of 3-[4-[2-(2,6-dioxo-3-piperidyl)-3-oxo-isoindolin-5-yl]piperazin-1-yl]propanoic acid (38 mg, 0.09 mmol), N-[1-[5-(3-cyano-6-ethoxy-pyrazolo[1,5-a]pyridin-4-yl)-2-pyridyl]-4-(piperazin-1-ylmethyl)-4-piperidyl]-2,5-difluoro-benzamide (57 mg, 0.09 mmol) in DMA (2 mL) were added T₃P (45.27 mg, 0.14 mmol), and DIEA (0.02 mL, 0.28 mmol). The mixture was stirred at rt for 2 h. Upon completion, the resulting solution was purified by Prep-HPLC to obtain the desired product (19.1 mg, 20.15% yield) as a white solid. ¹H NMR (300 MHz, DMSO-d6) δ 8.63 (d, J=2.3 Hz, 1H), 8.56 (s, 1H), 8.32 (d, J=2.3 Hz, 1H), 7.91-7.79 (m, 1H), 7.44 (d, J=8.4 Hz, 1H), 7.39-7.31 (m, 3H), 7.31-7.23 (m, 2H), 7.17 (d, J=2.3 Hz, 1H), 6.98 (d, J=8.9 Hz, 1H), 5.19-4.98 (m, 1H), 4.38-4.32 (m, 1H), 4.28-4.09 (m, 5H), 3.49-3.45 (m, 5H), 3.22-3.09 (m, 6H), 2.98-2.81 (m, 1H), 2.74-2.72 (m, 2H), 2.66-2.53 (m, 8H), 2.46-2.42 (m, 4H), 2.42-2.28 (m, 3H), 2.07-1.95 (m, 1H), 1.62-1.58 (m, 2H), 1.39 (t, J=6.9 Hz, 3H). Analytical LCMS ESI-MS(+) m/z 984 [M+H]⁺, RT=1.60 min, purity: 95% (Method Q); Prep-HPLC purification conditions: XBridge Prep OBD C18 Column, 30×150 mm, 5 μm; Mobile Phase A: Water (10 mmol/L NH₄HCO₃+0.1% NH₃·H₂O), Mobile Phase B: ACN; Flow rate: 60 mL/min; Gradient: 42% B to 52% B in 8 min, 52% B; RT=7.85 min.

### Example 13

### N-(1-(5-(3-cyano-6-ethoxypyrazolo[1,5-a]pyridin-4-yl)pyridin-2-yl)-4-((4-(5-(4-(2-(2,6-dioxopiperidin-3-yl)-3-oxoisoindolin-5-yl)piperazin-1-yl)pentanoyl)piperazin-1-yl)methyl)piperidin-4-yl)-2,5-difluorobenzamide

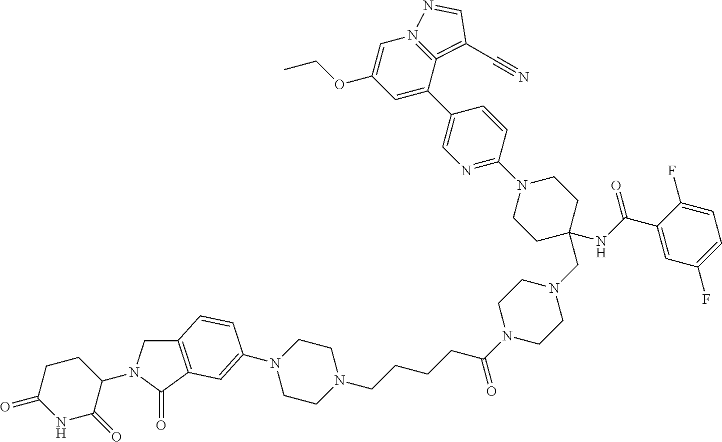

The title compound was prepared in 11.82% overall yield as a white solid according to the preparation of Example 5 using 3-(1-oxo-6-(piperazin-1-yl) isoindolin-2-yl)piperidine-2,6-dione in STEP C. ¹H NMR (400 MHz, DMSO-d6) δ 8.55-8.47 (m, 2H), 8.27 (d, J=2.6 Hz, 1H), 8.24 (s, 1H), 7.73 (dd, J=8.8, 2.6 Hz, 1H), 7.47-7.45 (m, 1H), 7.34-7.24 (m, 4H), 7.22-7.20 (m, 2H), 6.95 (d, J=9.0 Hz, 1H), 5.01 (dd, J=13.3, 5.1 Hz, 1H), 4.35 (d, J=17.1 Hz, 1H), 4.22 (d, J=17.1 Hz, 1H), 4.14-4.08 (m, 4H), 3.43-3.36 (m, 4H), 3.30-3.24 (m, 4H), 3.11 (t, J=12.4 Hz, 2H), 2.87-2.80 (m, 5H), 2.73-2.58 (m, 6H), 2.48-2.43 (m, 3H), 2.35-2.27 (m, 5H), 2.05-1.97 (m, 1H), 1.65-1.45 (m, 6H), 1.35 (t, J=6.9 Hz, 3H); Analytical LCMS ESI-MS(+) m/z 1011.6 [M+H]⁺, RT=1.43 min, purity: 95.6% (Method Q); Prep-HPLC purification conditions: Column: Xselect CSH F-Phenyl OBD Column 19×150 mm 5 μm; Mobile Phase A: water (0.1% FA), Mobile Phase B: ACN; Flow rate: 25 mL/min; Gradient: 10% B to 32% B in 8 min, 32% B; RT-7.67 min.

### Example 14

### N-(1-(5-(3-cyano-6-ethoxypyrazolo[1,5-a]pyridin-4-yl)pyridin-2-yl)-4-((4-(4-(4-(2-(2,6-dioxopiperidin-3-yl)-1-oxoisoindolin-5-yl)piperazin-1-yl)butanoyl)piperazin-1-yl)methyl)piperidin-4-yl)-2,5-difluorobenzamide

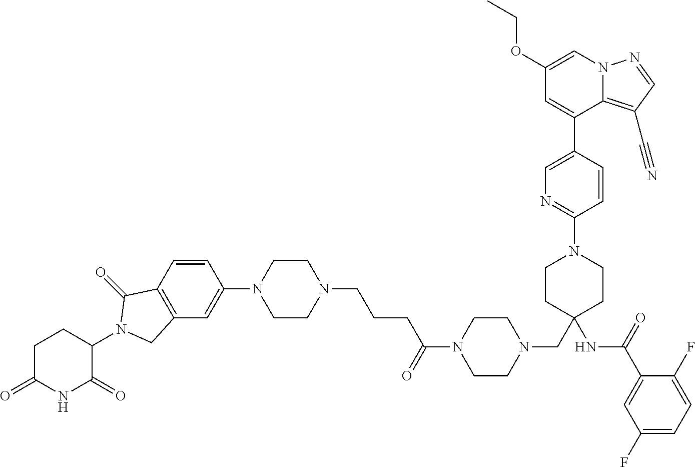 [SMILES: CCOc1cc(-c2ccc(N3CCC(CN4CCN(C(=O)CCCN5CCN(c6ccc7c(c6)CN(C6CCC(=O)NC6=O)C7=O)CC5)CC4)(NC(=O)c4cc(F)ccc4F)CC3)nc2)c2c(C#N)cnn2c1]

The title compound was prepared in 12.2% overall yield as a white solid according to the preparation of Example 6 using tert-butyl 4-oxobutanoate in STEP B. ¹H NMR (300 MHz, DMSO-d6) δ 8.64 (d, J=2.5 Hz, 1H), 8.57 (s, 1H), 8.32 (d, J=2.6 Hz, 1H), 7.81-7.72 (m, 1H), 7.52 (d, J=9.1 Hz, 1H), 7.43-7.31 (m, 3H), 7.27 (d, J=2.5 Hz, 1H), 7.05-7.03 (m, 2H), 6.97 (d, J=9.0 Hz, 1H), 5.04 (dd, J=13.2, 5.1 Hz, 1H), 4.33-4.30 (m, 1H), 4.25-4.05 (m, 5H), 3.38 (d, J=2.5 Hz, 8H), 3.26 (d, J=5.8 Hz, 4H), 3.15 (t, J=12.4 Hz, 2H), 2.98-2.79 (m, 1H), 2.73-2.71 (m, 2H), 2.61-2.58 (m, 1H), 2.47 (s, 3H), 2.41-2.24 (m, 8H), 2.09-1.92 (m, 1H), 1.78-1.57 (m, 4H), 1.38 (t, J=6.9 Hz, 3H); LCMS ESI-MS(+) m/z 998 [M+H]⁺, RT=1.44 min, purity: 97.2% (Method Q); Prep-HPLC conditions: Column: XSelect CSH Prep C18 OBD Column, 19×250 mm, 5 μm; Mobile Phase A: Water (10 mmol/L NH₄HCO₃+0.1% NH₃·H₂O), Mobile Phase B: ACN; Flow rate: 60 mL/min; Gradient: 47% B to 50% B in 12 min, 50% B; RT=10.5 min. Using similar procedures as described in Examples 1-14, the following titled examples 15-30 were obtained.

### Example 15

### N-[1-(5-{3-cyano-6-ethoxypyrazolo[1,5-a]pyridin-4-yl}pyridin-2-yl)-4-{[4-(3-{4-[2-(2,6-dioxopiperidin-3-yl)-1-oxo-2,3-dihydro-1H-isoindol-5-yl]piperidin-1-yl}propanoyl)piperazin-1-yl]methyl}piperidin-4-yl]-2,5-difluorobenzamide

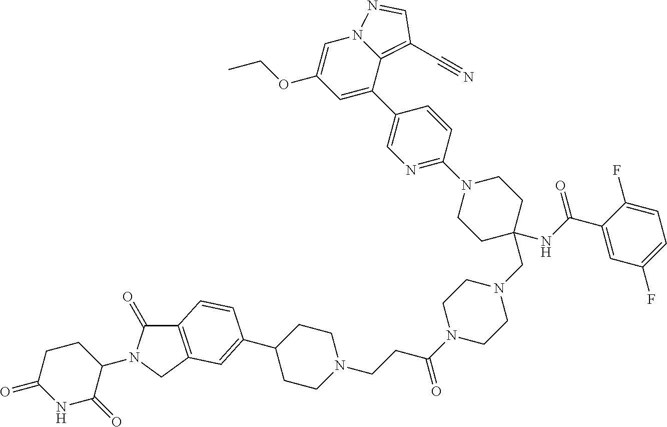 [SMILES: CCOc1cc(-c2ccc(N3CCC(CN4CCN(C(=O)CCN5CCC(c6ccc7c(c6)CN(C6CCC(=O)NC6=O)C7=O)CC5)CC4)(NC(=O)c4cc(F)ccc4F)CC3)nc2)c2c(C#N)cnn2c1]

The title compound was prepared as a white solid according to the preparation of Example 1 using 3-(1-oxo-5-(piperidin-4-yl) isoindolin-2-yl)piperidine-2,6-dione, HCl and tert-butyl 3-bromopropanoate in STEP H. ¹H NMR (400 MHz, DMSO-d₆) δ 10.96 (s, 1H), 8.65 (d, J=2.0 Hz, 1H), 8.57 (s, 1H), 8.32 (d, J=2.7 Hz, 1H), 8.19-8.08 (m, 1H), 7.75 (dd, J=8.8, 2.6 Hz, 1H), 7.64 (d, J=7.8 Hz, 1H), 7.48 (s, 1H), 7.42-7.29 (m, 4H), 7.27 (d, J=2.1 Hz, 1H), 6.97 (d, J=9.2 Hz, 1H), 5.14-5.05 (m, 1H), 4.46-4.25 (m, 2H), 4.24-4.12 (m, 4H), 3.43 (br d, J=1.1 Hz, 4H), 3.22-3.11 (m, 2H), 3.03-2.84 (m, 3H), 2.74 (s, 2H), 2.69-2.52 (m, 10H), 2.41-2.29 (m, 3H), 2.11-1.93 (m, 3H), 1.80-1.57 (m, 6H), 1.38 (t, J=7.0 Hz, 3H); Analytical LCMS: m/z 982.3 [M+H]⁺, RT=1.238 min (UHPLC Method D). Prep-HPLC purification condition: Column: Phen Axia C18 5μ 30×100 mm; Flow rate: 40 mL/min flow rate; Mobile Phase A: 95% H2O/5% MeCN with 0.01M NH4OAc. Mobile Phase B: 5% H2O/95% MeCN with 0.01M NH4OAc; Gradient: 20-80% B (40 min); Elution: 20.8-22.8 minutes; Number of runs: 2.

### Example 16

### N-[1-(5-{3-cyano-6-ethoxypyrazolo[1,5-a]pyridin-4-yl}pyridin-2-yl)-4-{[4-(7-{4-[2-(2,6-dioxopiperidin-3-yl)-3-oxo-2,3-dihydro-1H-isoindol-5-yl]piperazin-1-yl}heptanoyl)piperazin-1-yl]methyl}piperidin-4-yl]-2,5-difluorobenzamide

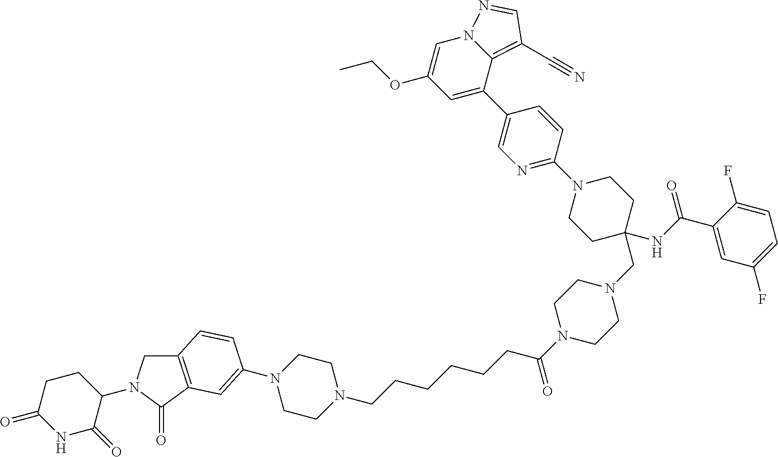 [SMILES: CCOc1cc(-c2ccc(N3CCC(CN4CCN(C(=O)CCCCCCN5CCN(c6ccc7c(c6)C(=O)N(C6CCC(=O)NC6=O)C7)CC5)CC4)(NC(=O)c4cc(F)ccc4F)CC3)nc2)c2c(C#N)cnn2c1]

The title compound was prepared as a white solid according to the preparation of Example 1 using 3-(1-oxo-6-(piperazin-1-yl) isoindolin-2-yl)piperidine-2,6-dione, HCl in STEP H. ¹H NMR (400 MHz, DMSO-d₆) δ 10.93 (s, 1H), 8.64 (d, J=2.0 Hz, 1H), 8.57 (s, 1H), 8.32 (d, J=2.7 Hz, 1H), 8.12 (s, 1H), 7.75 (dd, J=8.8, 2.6 Hz, 1H), 7.51 (d, J=8.9 Hz, 1H), 7.34 (ddq, J=7.8, 5.1, 2.6 Hz, 3H), 7.27 (d, J=2.0 Hz, 1H), 7.08-7.02 (m, 2H), 6.97 (d, J=8.8 Hz, 1H), 5.09-5.00 (m, 1H), 4.36-4.28 (m, 1H), 4.24-4.12 (m, 5H), 3.45-3.35 (m, 4H), 3.29-3.24 (m, 4H), 3.22-3.10 (m, 2H), 2.96-2.83 (m, 1H), 2.72 (s, 2H), 2.62-2.54 (m, 1H), 2.41-2.23 (m, 8H), 2.01-1.92 (m, 1H), 1.68-1.56 (m, 2H), 1.54-1.42 (m, 4H), 1.38 (t, J=6.9 Hz, 3H), 1.33-1.25 (m, 4H)—some product peaks obscured by DMSO signal; Analytical LCMS: m/z 1039.5 [M+H]⁺, RT=1.254 min (UHPLC Method D). Prep-HPLC purification condition: Column: Phen Axia C18 5μ 30×100 mm; Flow rate: 40 mL/min flow rate; Mobile Phase A: 95% H2O/5% MeCN with 0.01M NH4OAc. Mobile Phase B: 5% H2O/95% MeCN with 0.01M NH4OAc; Gradient: 10-100% B (40 min); Elution: 25.5-27.1 minutes; Number of runs: 3.

### Example 17

### N-[1-(5-{3-cyano-6-ethoxypyrazolo[1,5-a]pyridin-4-yl}pyridin-2-yl)-4-{[4-(3-{4-[2-(2,6-dioxopiperidin-3-yl)-3-oxo-2,3-dihydro-1H-isoindol-5-yl]piperidin-1-yl}propanoyl)piperazin-1-yl]methyl}piperidin-4-yl]-2,5-difluorobenzamide

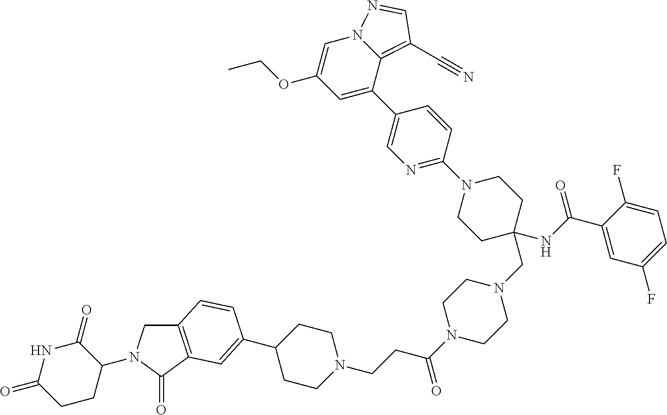 [SMILES: CCOc1cc(-c2ccc(N3CCC(CN4CCN(C(=O)CCN5CCC(c6ccc7c(c6)C(=O)N(C6CCC(=O)NC6=O)C7)CC5)CC4)(NC(=O)c4cc(F)ccc4F)CC3)nc2)c2c(C#N)cnn2c1]

The title compound was prepared as a white solid according to the preparation of Example 1 using 3-(1-oxo-6-(piperidin-4-yl) isoindolin-2-yl)piperidine-2,6-dione, HCl and tert-butyl 3-bromopropanoate in STEP H. ¹H NMR (400 MHz, DMSO-d6) δ 10.96 (br s, 1H), 8.64 (d, J=2.0 Hz, 1H), 8.57 (s, 1H), 8.32 (d, J=2.9 Hz, 1H), 8.13 (s, 1H), 7.75 (dd, J=8.9, 2.6 Hz, 1H), 7.58-7.49 (m, 3H), 7.39-7.30 (m, 3H), 7.27 (d, J=2.1 Hz, 1H), 6.97 (d, J=9.1 Hz, 1H), 5.10 (dd, J=13.3, 5.1 Hz, 1H), 4.45-4.36 (m, 1H), 4.34-4.24 (m, 1H), 4.23-4.11 (m, 4H), 3.50-3.36 (m, 4H), 3.22-3.11 (m, 2H), 3.02-2.84 (m, 3H), 2.74 (s, 2H), 2.68-2.53 (m, 6H), 2.49-2.29 (m, 7H), 2.12-1.94 (m, 3H), 1.82-1.74 (m, 2H), 1.71-1.55 (m, 4H), 1.38 (t, J=6.9 Hz, 3H); Analytical LCMS: m/z 982.3 [M+H]⁺, RT=1.309 min (UHPLC Method D). Prep-HPLC purification condition: Column: Phen Axia C18 5μ 30×100 mm; Flow rate: 40 mL/min flow rate; Mobile Phase A: 95% H2O/5% MeCN with 0.01M NH4OAc. Mobile Phase B: 5% H2O/95% MeCN with 0.01M NH4OAc; Gradient: 10-100% B (40 min); Elution: 20.1-24.5 minutes; Number of runs: 5.

### Example 18

### N-[1-(5-{3-cyano-6-ethoxypyrazolo[1,5-a]pyridin-4-yl}pyridin-2-yl)-4-{[4-(5-{4-[2-(2,6-dioxopiperidin-3-yl)-3-oxo-2,3-dihydro-1H-isoindol-5-yl]piperidin-1-yl}pentanoyl)piperazin-1-yl]methyl}piperidin-4-yl]-2,5-difluorobenzamide

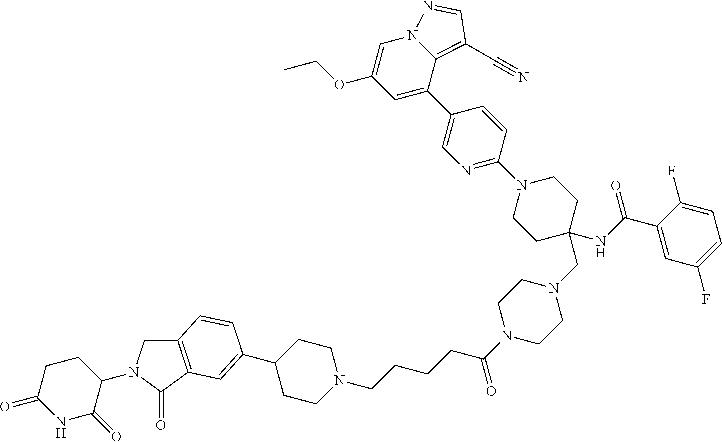 [SMILES: CCOc1cc(-c2ccc(N3CCC(CN4CCN(C(=O)CCCCN5CCC(c6ccc7c(c6)C(=O)N(C6CCC(=O)NC6=O)C7)CC5)CC4)(NC(=O)c4cc(F)ccc4F)CC3)nc2)c2c(C#N)cnn2c1]

### Example 19

### N-[1-(5-{3-cyano-6-ethoxypyrazolo[1,5-a]pyridin-4-yl}pyridin-2-yl)-4-{[4-(7-{4-[2-(2,6-dioxopiperidin-3-yl)-3-oxo-2,3-dihydro-1H-isoindol-5-yl]piperidin-1-yl}heptanoyl)piperazin-1-yl]methyl}piperidin-4-yl]-2,5-difluorobenzamide

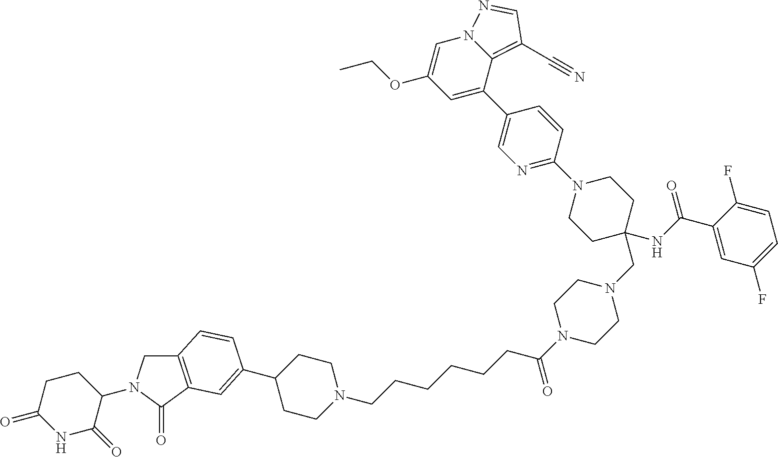 [SMILES: CCOc1cc(-c2ccc(N3CCC(CN4CCN(C(=O)CCCCCCN5CCC(c6ccc7c(c6)C(=O)N(C6CCC(=O)NC6=O)C7)CC5)CC4)(NC(=O)c4cc(F)ccc4F)CC3)nc2)c2c(C#N)cnn2c1]

### Example 20

### N-[1-(5-{3-cyano-6-ethoxypyrazolo[1,5-a]pyridin-4-yl}pyridin-2-yl)-4-{[4-(7-{[4-(2,6-dioxopiperidin-3-yl)phenyl]amino}heptanoyl)piperazin-1-yl]methyl}piperidin-4-yl]-2,5-difluorobenzamide

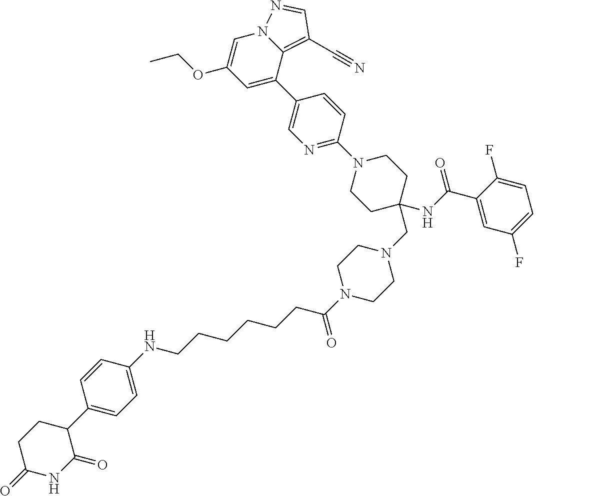 [SMILES: CCOc1cc(-c2ccc(N3CCC(CN4CCN(C(=O)CCCCCCNc5ccc(C6CCC(=O)NC6=O)cc5)CC4)(NC(=O)c4cc(F)ccc4F)CC3)nc2)c2c(C#N)cnn2c1]

### Example 21

### (N-[1-(5-{3-cyano-6-ethoxypyrazolo[1,5-a]pyridin-4-yl}pyridin-2-yl)-4-{[4-(9-{[4-(2,6-dioxopiperidin-3-yl)phenyl]amino}nonanoyl)piperazin-1-yl]methyl}piperidin-4-yl]-2,5-difluorobenzamide

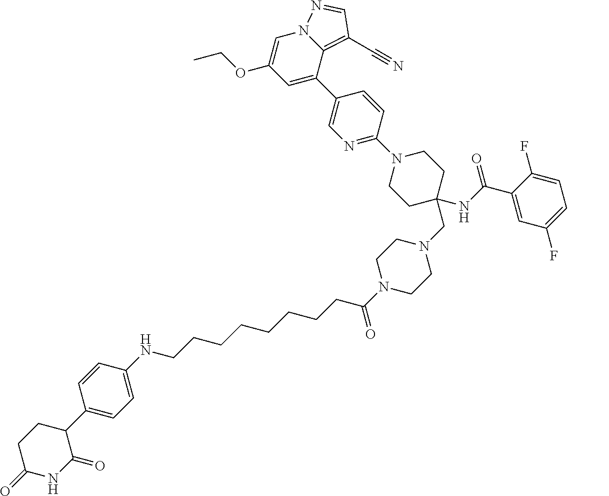 [SMILES: CCOc1cc(-c2ccc(N3CCC(CN4CCN(C(=O)CCCCCCCCNc5ccc(C6CCC(=O)NC6=O)cc5)CC4)(NC(=O)c4cc(F)ccc4F)CC3)nc2)c2c(C#N)cnn2c1]

### Example 22

### N-[1-(5-{3-cyano-6-ethoxypyrazolo[1,5-a]pyridin-4-yl}pyridin-2-yl)-4-({4-[7-({3-[(2,6-dioxopiperidin-3-yl)amino]phenyl}amino)heptanoyl]piperazin-1-yl}methyl)piperidin-4-yl]-2,5-difluorobenzamide

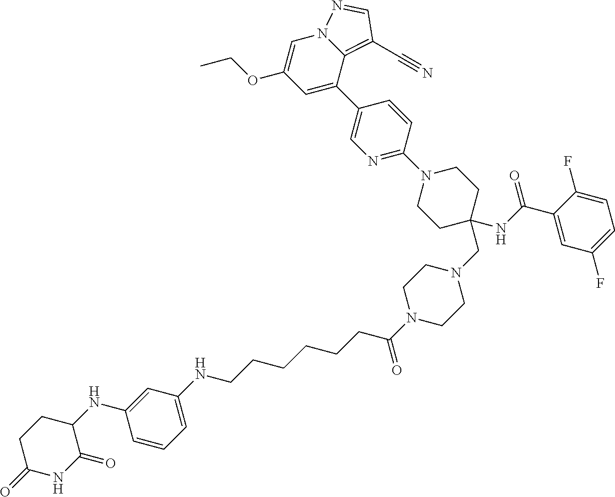 [SMILES: CCOc1cc(-c2ccc(N3CCC(CN4CCN(C(=O)CCCCCCNc5cccc(NC6CCC(=O)NC6=O)c5)CC4)(NC(=O)c4cc(F)ccc4F)CC3)nc2)c2c(C#N)cnn2c1]

### Example 23

### N-[1-(5-{3-cyano-6-ethoxypyrazolo[1,5-a]pyridin-4-yl}pyridin-2-yl)-4-({4-[9-({3-[(2,6-dioxopiperidin-3-yl)amino]phenyl}amino) nonanoyl]piperazin-1-yl}methyl)piperidin-4-yl]-2,5-difluorobenzamide

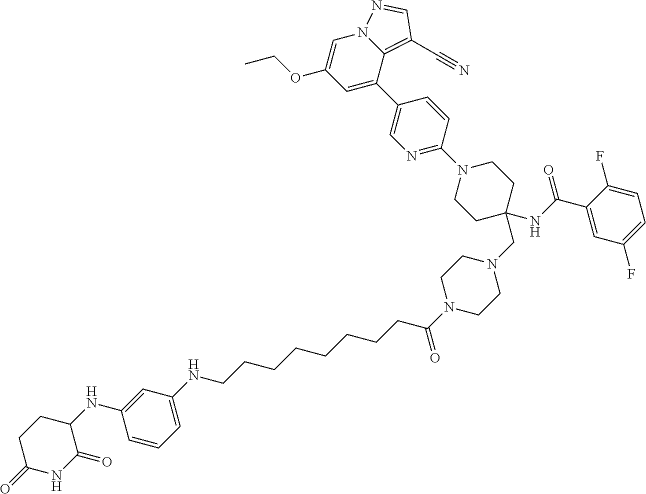 [SMILES: CCOc1cc(-c2ccc(N3CCC(CN4CCN(C(=O)CCCCCCCCNc5cccc(NC6CCC(=O)NC6=O)c5)CC4)(NC(=O)c4cc(F)ccc4F)CC3)nc2)c2c(C#N)cnn2c1]

### Example 24

### N-[1-(5-{3-cyano-6-ethoxypyrazolo[1,5-a]pyridin-4-yl}pyridin-2-yl)-4-{[4-(9-{[3-(2,4-dioxo-1,3-diazinan-1-yl)-1-methyl-1H-indazol-6-yl]amino}nonanoyl)piperazin-1-yl]methyl}piperidin-4-yl]-2,5-difluorobenzamide

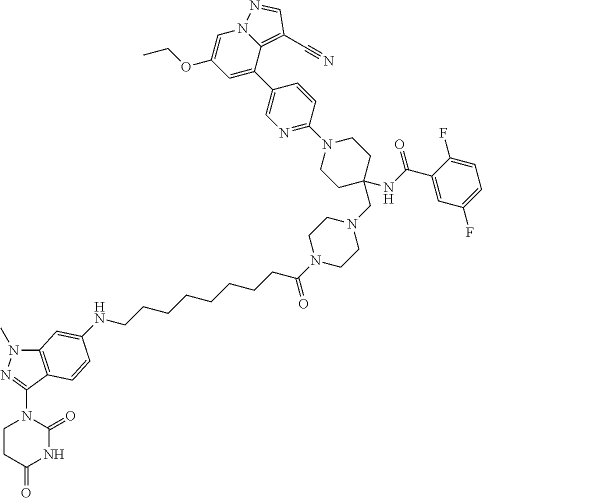 [SMILES: CCOc1cc(-c2ccc(N3CCC(CN4CCN(C(=O)CCCCCCCCNc5ccc6c(N7CCC(=O)NC7=O)nn(C)c6c5)CC4)(NC(=O)c4cc(F)ccc4F)CC3)nc2)c2c(C#N)cnn2c1]

The title compound was prepared as a solid according to the preparation of Example 1 using 1-(6-amino-1-methyl-1H-indazol-3-yl)dihydropyrimidine-2,4(1H,3H)-dione and tert-butyl 9-bromononanoate in STEP H. ¹H NMR (400 MHz, DMSO-d₆) δ 10.44 (br d, J=0.8 Hz, 1H), 8.65 (d, J=2.1 Hz, 1H), 8.57 (s, 1H), 8.32 (d, J=2.8 Hz, 1H), 8.12 (s, 1H), 7.75 (dd, J=8.9, 2.6 Hz, 1H), 7.37-7.31 (m, 3H), 7.28-7.24 (m, 2H), 6.97 (d, J=8.9 Hz, 1H), 6.51 (dd, J=8.9, 1.8 Hz, 1H), 6.29 (d, J=1.7 Hz, 1H), 5.90 (t, J=5.4 Hz, 1H), 4.23-4.12 (m, 4H), 3.85 (t, J=6.7 Hz, 2H), 3.80 (s, 3H), 3.45-3.36 (m, 4H), 3.21-3.11 (m, 2H), 3.09-3.01 (m, 2H), 2.74-2.66 (m, 4H), 2.48-2.43 (m, 3H), 2.37-2.29 (m, 2H), 2.26 (br t, J=7.5 Hz, 2H), 1.66-1.56 (m, 4H), 1.52-1.44 (m, 2H), 1.42-1.22 (m, 12H); Analytical LCMS: m/z 998.7 [M+H]⁺, RT=1.459 min (UHPLC Method D). Prep-HPLC purification condition: Column: Phen Axia C18 5μ 30×100 mm; Flow rate: 40 mL/min flow rate; Mobile Phase A: 95% H2O/5% MeCN with 0.01M NH4OAc. Mobile Phase B: 5% H2O/95% MeCN with 0.01M NH4OAc; Gradient: 20-100% B (40 min); Elution: 23.7-25.5 minutes; Number of runs: 5.

### Example 25

### N-[1-(5-{3-cyano-6-ethoxypyrazolo[1,5-a]pyridin-4-yl}pyridin-2-yl)-4-{[4-(9-{[3-(2,6-dioxopiperidin-3-yl)-1-methyl-1H-indazol-6-yl]amino}nonanoyl)piperazin-1-yl]methyl}piperidin-4-yl]-2,5-difluorobenzamide

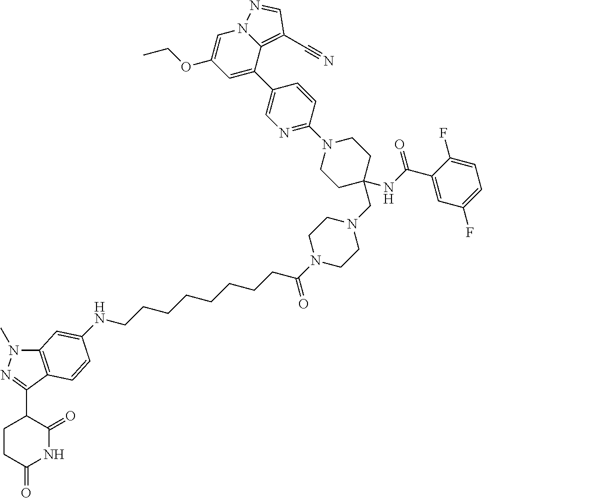 [SMILES: CCOc1cc(-c2ccc(N3CCC(CN4CCN(C(=O)CCCCCCCCNc5ccc6c(C7CCC(=O)NC7=O)nn(C)c6c5)CC4)(NC(=O)c4cc(F)ccc4F)CC3)nc2)c2c(C#N)cnn2c1]

### Example 26

### N-[1-(5-{3-cyano-6-ethoxypyrazolo[1,5-a]pyridin-4-yl}pyridin-2-yl)-4-({4-[5-(2-{[2-(2,6-dioxopiperidin-3-yl)-1-oxo-2,3-dihydro-1H-isoindol-4-yl]oxy}acetamido)pentanoyl]piperazin-1-yl}methyl)piperidin-4-yl]-2,5-difluorobenzamide

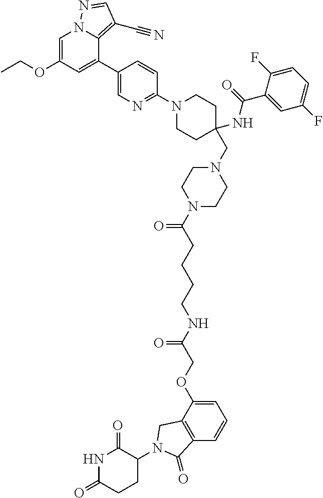 [SMILES: CCOc1cc(-c2ccc(N3CCC(CN4CCN(C(=O)CCCCNC(=O)COc5cccc6c5CN(C5CCC(=O)NC5=O)C6=O)CC4)(NC(=O)c4cc(F)ccc4F)CC3)nc2)c2c(C#N)cnn2c1]

### Example 27

### N-[1-(5-{3-cyano-6-ethoxypyrazolo[1,5-a]pyridin-4-yl}pyridin-2-yl)-4-({4-[7-(2-{[2-(2,6-dioxopiperidin-3-yl)-1-oxo-2,3-dihydro-1H-isoindol-4-yl]oxy}acetamido)heptanoyl]piperazin-1-yl}methyl)piperidin-4-yl]-2,5-difluorobenzamide

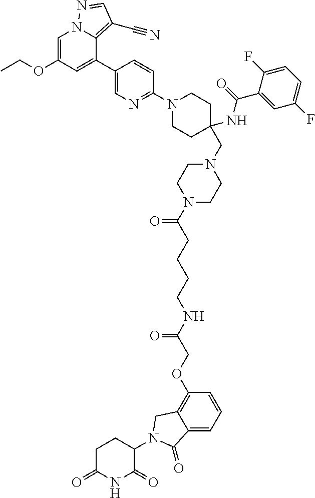 [SMILES: CCOc1cc(-c2ccc(N3CCC(CN4CCN(C(=O)CCCCNC(=O)COc5cccc6c5CN(C5CCC(=O)NC5=O)C6=O)CC4)(NC(=O)c4cc(F)ccc4F)CC3)nc2)c2c(C#N)cnn2c1]

### Example 28

### N-[1-(5-{3-cyano-6-ethoxypyrazolo[1,5-a]pyridin-4-yl}pyridin-2-yl)-4-({4-[11-(2-{[2-(2,6-dioxopiperidin-3-yl)-1-oxo-2,3-dihydro-1H-isoindol-4-yl]oxy}acetamido)undecanoyl]piperazin-1-yl}methyl)piperidin-4-yl]-2,5-difluorobenzamide

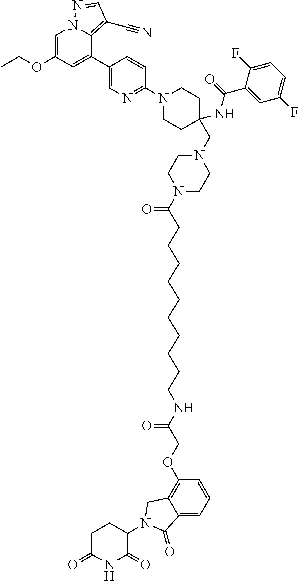 [SMILES: CCOc1cc(-c2ccc(N3CCC(CN4CCN(C(=O)CCCCCCCCCCNC(=O)COc5cccc6c5CN(C5CCC(=O)NC5=O)C6=O)CC4)(NC(=O)c4cc(F)ccc4F)CC3)nc2)c2c(C#N)cnn2c1]

### Example 29

### N-[1-(5-{3-cyano-6-ethoxypyrazolo[1,5-a]pyridin-4-yl}pyridin-2-yl)-4-({4-[13-(2-{[2-(2,6-dioxopiperidin-3-yl)-1-oxo-2,3-dihydro-1H-isoindol-4-yl]oxy}acetamido)tridecanoyl]piperazin-1-yl}methyl)piperidin-4-yl]-2,5-difluorobenzamide

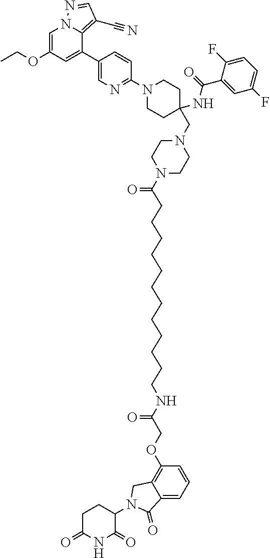 [SMILES: CCOc1cc(-c2ccc(N3CCC(CN4CCN(C(=O)CCCCCCCCCCCCNC(=O)COc5cccc6c5CN(C5CCC(=O)NC5=O)C6=O)CC4)(NC(=O)c4cc(F)ccc4F)CC3)nc2)c2c(C#N)cnn2c1]

### Example 30

### N-[1-(5-{3-cyano-6-ethoxypyrazolo[1,5-a]pyridin-4-yl}pyridin-2-yl)-4-({4-[15-(2-{[2-(2,6-dioxopiperidin-3-yl)-1-oxo-2,3-dihydro-1H-isoindol-4-yl]oxy}acetamido)pentadecanoyl]piperazin-1-yl}methyl)piperidin-4-yl]-2,5-difluorobenzamide

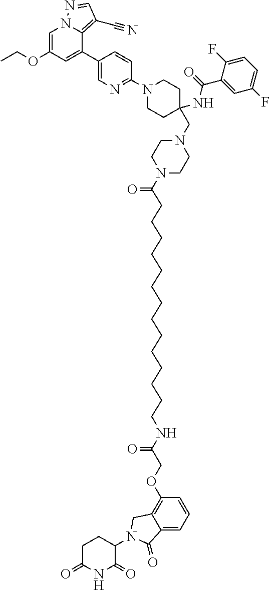 [SMILES: CCOc1cc(-c2ccc(N3CCC(CN4CCN(C(=O)CCCCCCCCCCCCCCNC(=O)COc5cccc6c5CN(C5CCC(=O)NC5=O)C6=O)CC4)(NC(=O)c4cc(F)ccc4F)CC3)nc2)c2c(C#N)cnn2c1]

| | Analytical | |
|---|---|---|
| | LCMS | |
| | ESI-MS(+) | |
| | m/z/RT | |
| | (min) | |
| Example # | (Method); | ¹H NMR |
| Example | 1010.5 | ¹H NMR (500 MHz, DMSO-d₆) δ 8.64 (s, 1H), 8.57 (s, 1H), 8.36- |
| 18. | [M + H]⁺, | 8.31 (m, 1H), 8.14 (s, 1H), 7.76 (dd, J = 8.7, 2.3 Hz, 1H), 7.59- |
| | RT = 1.85 | 7.50 (m, 3H), 7.38-7.29 (m, 3H), 7.27 (s, 1H), 6.98 (br d, J = 8.9 |
| | min (A) | Hz, 1H), 5.10 (br dd, J = 13.1, 4.9 Hz, 1H), 4.41 (br d, J = 17.1 Hz, |
| | | 1H), 4.29 (br d, J = 17.1 Hz, 1H), 4.23-4.12 (m, 3H), 3.25-3.10 |
| | | (m, 1H), 3.03-2.87 (m, 4H), 2.74 (s, 1H), 2.65-2.58 (m, 2H), |
| | | 2.55 (DMSO), 2.50-2.45 (m, 2H), 2.45-2.26 (m, 6H), 2.10- |
| | | 1.90 (m, 3H), 1.81-1.72 (m, 3H), 1.72-1.58 (m, 4H), 1.54-1.44 |
| | | (m, 4H), 1.39 (t, J = 6.9 Hz, 3H) |
| Example | 1038.3 | ¹H NMR (500 MHz, DMSO-d₆) δ 10.99 (s, 1H), 8.66 (d, J = 1.5 |
| 19. | [M + H]⁺, | Hz, 1H), 8.62-8.51 (m, 1H), 8.36 (d, J = 2.1 Hz, 1H), 7.83 (dd, |
| | RT = 1.44 | J = 9.0, 2.3 Hz, 1H), 7.63-7.56 (m, 2H), 7.56-7.47 (m, 1H), 7.40 |
| | min (B) | (dt, J = 9.5, 4.7 Hz, 2H), 7.32-7.24 (m, 1H), 7.07 (br d, J = 10.4 |
| | | Hz, 1H), 5.10 (br dd, J = 13.4, 4.9 Hz, 1H), 4.49-4.39 (m, 1H), |
| | | 4.36-4.23 (m, 2H), 4.22-4.11 (m, 2H), 3.61 (br d, J = 8.9 Hz, |
| | | 1H), 3.22 (br t, J = 12.5 Hz, 2H), 3.12-2.97 (m, 3H), 2.97-2.85 |
| | | (m, 1H), 2.66-2.58 (m, 1H), 2.55 (DMSO), 2.50-2.29 (m, 4H), |
| | | 2.10-1.88 (m, 4H), 1.80-1.61 (m, 3H), 1.53 (m, 2H), 1.42-1.30 |
| | | (m, 5H). |
| Example | 915.6 | ¹H NMR (500 MHz, DMSO-d₆) δ 10.71 (s, 1H), 8.61 (s, 1H), |
| 20. | [M + H]⁺, | 8.55 (s, 1H), 8.32 (s, 1H), 8.14 (s, 1H), 7.76 (d, J = 8.5 Hz, 1H), |
| | RT = 2.2 | 7.38-7.30 (m, 3H), 7.26 (s, 1H), 6.97 (br d, J = 9.2 Hz, 1H), 6.90 |
| | min (A) | (br d, J = 8.2 Hz, 2H), 6.50 (br d, J = 7.9 Hz, 2H), 5.46-5.42 (m, |
| | | 1H), 4.21-4.12 (m, 4H), 3.63 (br dd, J = 10.7, 4.9 Hz, 1H), 3.59- |
| | | 3.39 (m, 4H), 3.20-3.12 (m, 2H), 2.96 (q, J = 6.5 Hz, 2H), 2.73 |
| | | (br. s, 2H), 2.64-2.57 (m, 1H), 2.55 (DMSO), 2.49-2.41 (m, |
| | | 3H), 2.40-2.21 (m, 4H), 2.13-1.97 (m, 2H), 1.66-1.57 (m, 2H), |
| | | 1.55-1.44 (m, 4H), 1.41-1.23 (m, 8H) |
| Example | 943.0 | ¹H NMR (500 MHz, DMSO-d₆) δ 10.71 (s, 1H), 8.62 (s, 1H), |
| | [M + H]⁺, | 8.56 (s, 1H), 8.32 (s, 1H), 8.19-8.08 (m, 1H), 7.75 (d, J = 8.4 Hz, |
| 21 | RT = 2.34 | 1H), 7.38-7.29 (m, 3H), 7.26 (s, 1H), 6.97 (br d, J = 8.9 Hz, 1H), |
| | min (A) | 6.93-6.85 (m, J = 8.2 Hz, 2H), 6.55-6.46 (m, J = 8.2 Hz, 2H), |
| | | 4.24-4.09 (m, 4H), 3.74-3.35 (m, 3H), 3.25-3.06 (m, 2H), 2.96 |
| | | (br t, J = 5.5 Hz, 2H), 2.73 (br s, 1H), 2.66-2.57 (m, 1H), 2.55 |
| | | (DMSO), 2.49-2.41 (m, 2H), 2.40-2.21 (m, 4H), 2.17-1.95 (m, |
| | | 2H), 1.71-1.57 (m, 2H), 1.55-1.43 (m, 4H), 1.40-1.29 (m, 6H), |
| | | 1.27 (br s, 6H). |
| Example | 930.4 | ¹H NMR (500 MHz, DMSO-d₆) δ 10.75 (s, 1H), 8.63 (s, 1H), |
| 22 | [M + H]⁺, | 8.56 (s, 1H), 8.34-8.31 (m, 1H), 8.14 (s, 1H), 7.76 (d, J = 8.4 Hz, |
| | RT = 2.24 | 1H), 7.39-7.30 (m, 4H), 7.28-7.25 (m, 1H), 6.97 (br d, J = 9.2 |
| | min (A) | Hz, 1H), 6.77 (br t, J = 7.9 Hz, 1H), 5.94-5.84 (m, 3H), 5.43 (br d, |
| | | J = 7.3 Hz, 1H), 4.23-4.12 (m, 5H), 3.90 (s, 3H), 3.55-3.38 (m, |
| | | 3H), 3.20-3.12 (m, 2H), 2.97-2.88 (m, 2H), 2.77-2.68 (m, 3H), |
| | | 2.62-2.58 (m, 1H), 2.56-2.56 (m, 1H), 2.55 (DMSO), 2.49- |
| | | 2.41 (m, 2H), 2.39-2.21 (m, 4H), 2.15-2.07 (m, 1H), 1.89-1.80 |
| | | (m, 1H), 1.67-1.57 (m, 2H), 1.54-1.44 (m, 5H), 1.41-1.27 (m, |
| | | 8H) |
| Example | 958.70 | ¹H NMR (500 MHz, DMSO-d₆) δ 8.62 (s, 1H), 8.56 (s, 1H), 8.32 |
| 23 | [M + H]⁺, | (s, 1H), 8.14 (s, 1H), 7.76 (br d, J = 8.2 Hz, 1H), 7.33 (br d, J = 4.6 |
| | RT = 2.34 | Hz, 4H), 7.26 (s, 1H), 6.97 (br d, J = 8.9 Hz, 1H), 6.77 (t, J = 7.7 |
| | min (A) | Hz, 1H), 5.92-5.85 (m, 3H), 5.42 (br d, J = 7.3 Hz, 1H), 4.23- |
| | | 4.13 (m, 5H), 3.90 (s, 2H), 3.54-3.39 (m, 2H), 3.20-3.12 (m, |
| | | 2H), 2.93 (br d, J = 4.9 Hz, 2H), 2.76-2.68 (m, 3H), 2.60 (m, 1H), |
| | | 2.55 (DMSO), 2.49-2.44 (m, 2H), 2.38-2.23 (m, 4H), 2.15- |
| | | 2.07 (m, 1H), 1.89-1.80 (m, 1H), 1.66-1.57 (m, 2H), 1.54-1.43 |
| | | (m, 5H), 1.41-1.24 (m, 13H) |
| Example | 997.4 | ¹H NMR (500 MHz, DMSO-d₆) δ 10.81 (s, 1H), 8.63 (s, 1H), |
| 25 | [M + H]⁺, | 8.56 (s, 1H), 8.39-8.30 (m, 1H), 8.14 (br s, 1H), 7.86-7.70 (m, |
| | RT = 2.31 | 1H), 7.40-7.29 (m, 4H), 7.26 (s, 1H), 6.M1997 (br d, J = 8.9 Hz, |
| | min (A) | 1H), 6.53 (br d, J = 8.5 Hz, 1H), 6.31 (s, 1H), 4.26-4.08 (m, 4H), |
| | | 3.96-3.82 (m, 1H), 3.50 (br s, 1H), 3.16 (br t, J = 11.6 Hz, 2H), |
| | | 3.05 (br s, 2H), 2.73 (br s, 1H), 2.65-2.57 (m, 2H), 2.55 (s, |
| | | DMSO), 2.49-2.41 (m, 1H), 2.33 (br d, J = 11.9 Hz, 2H), 2.30- |
| | | 2.21 (m, 3H), 2.20-1.98 (m, 1H), 1.70-1.53 (m, 4H), 1.47 (br s, |
| | | 2H), 1.39 (br t, J = 6.9 Hz, 5H), 1.29 (br s, 5H) |
| Example | 1000.4 | ¹H NMR (500 MHz, DMSO-d₆) δ 8.63 (d, J = 1.9 Hz, 1H), 8.56 (s, |
| 26 | [M + H]⁺, | 1H), 8.33 (d, J = 2.4 Hz, 1H), 8.14-8.09 (m, 2H), 7.76 (dd, J = 8.9, |
| | RT = 1.41 | 2.4 Hz, 1H), 7.47 (t, J = 7.9 Hz, 1H), 7.34 (dt, J = 7.8, 3.9 Hz, 5H), |
| | min (B) | 7.26 (d, J = 2.0 Hz, 1H), 7.13 (d, J = 8.1 Hz, 1H), 6.97 (d, J = 8.9 Hz, |
| | | 1H), 5.13 (dd, J = 13.3, 5.1 Hz, 1H), 4.63-4.61 (m, 2H), 4.45 (s, |
| | | 1H), 4.37 (s, 1H), 4.21-4.13 (m, 5H), 3.19-3.11 (m, 4H), 3.00 |
| | | (s, 1H), 2.93-2.91 (m, 1H), 2.72 (s, 2H), 2.64-2.58 (m, 1H), |
| | | 2.55 (DMSO), 2.50-2.41 (m, 4H), 2.35-2.26 (m, 4H), 2.04- |
| | | 2.00 (m, 1H), 1.65-1.58 (m, 2H), 1.45 (br s, 4H), 1.39 (t, J = 6.9 |
| | | Hz, 4H) |
| Example | 1028.3 | ¹H NMR (500 MHz, DMSO-d₆) δ 10.99 (s, 1H), 8.65-8.59 (m, |
| 27 | [M + H]⁺, | 1H), 8.55 (s, 1H), 8.48 (br s, 1H), 8.34 (d, J = 2.4 Hz, 1H), 8.10 (br |
| | RT = 1.97 | t, J = 5.6 Hz, 1H), 7.80 (dd, J = 8.9, 2.4 Hz, 1H), 7.47 (br t, J = 7.7 |
| | min (A) | Hz, 2H), 7.42-7.33 (m, 3H), 7.29 (s, 1H), 7.26 (d, J = 1.9 Hz, 1H), |
| | | 7.18 (s, 1H), 7.16-7.01 (m, 3H), 5.10 (dd, J = 13.1, 5.0 Hz, 1H), |
| | | 4.62 (s, 2H), 4.47 (d, J = 17.6 Hz, 1H), 4.35 (d, J = 17.5 Hz, 1H), |
| | | 4.24 (br d, J = 12.1 Hz, 2H), 4.15 (q, J = 6.7 Hz, 2H), 3.23-3.08 |
| | | (m, 7H), 2.99-2.83 (m, 1H), 2.62 (br d, J = 17.5 Hz, 1H), 2.55 |
| | | (DMSO), 2.49-2.37 (m, 3H), 2.31 (br t, J = 7.2 Hz, 2H), 2.09- |
| | | 1.95 (m, 1H), 1.72 (br t, J = 11.2 Hz, 2H), 1.49-1.32 (m, 7H), |
| | | 1.30-1.15 (m, 4H) |
| Example | 1084.3 | ¹H NMR (500 MHz, DMSO-d₆) δ 11.00 (s, 1H), 8.64 (s, 1H), |
| 28 | [M + H]⁺, | 8.56 (s, 1H), 8.46 (br d, J = 1.9 Hz, 1H), 8.35 (d, J = 2.4 Hz, 1H), |
| | RT = 2.24 | 8.08 (t, J = 5.8 Hz, 1H), 7.80 (dd, J = 8.8, 2.4 Hz, 1H), 7.47 (br t, |
| | min (A) | J = 7.7 Hz, 2H), 7.43-7.33 (m, 3H), 7.30-7.24 (m, 2H), 7.17 (s, |
| | | 1H), 7.12 (d, J = 8.1 Hz, 1H), 7.06 (s, 1H), 7.04 (d, J = 8.9 Hz, 1H), |
| | | 5.12 (dd, J = 13.5, 5.1 Hz, 1H), 4.62 (s, 2H), 4.47 (d, J = 17.4 Hz, |
| | | 1H), 4.35 (d, J = 17.4 Hz, 1H), 4.25 (br d, J = 13.4 Hz, 2H), 4.16 (q, |
| | | J = 6.9 Hz, 2H), 3.22-3.15 (m, 4H), 3.11 (q, J = 6.7 Hz, 2H), 2.97- |
| | | 2.85 (m, 1H), 2.65-2.58 (m, 1H), 2.55 (DMSO), 2.49-2.37 (m, |
| | | 3H), 2.31 (br t, J = 7.4 Hz, 2H), 2.07-1.96 (m, 1H), 1.76-1.63 |
| | | (m, 2H), 1.52-1.44 (m, 2H), 1.44-1.34 (m, 5H), 1.29-1.16 (m, |
| | | 12H) |
| Example | 1112.6 | ¹H NMR (500 MHz, DMSO-d₆) δ 8.62 (d, J = 1.9 Hz, 1H), 8.55 (s, |
| 29 | [M + H]⁺, | 1H), 8.32 (d, J = 2.2 Hz, 1H), 8.13 (s, 1H), 8.06 (br t, J = 5.9 Hz, |
| | RT = 1.77 | 1H), 7.75 (dd, J = 8.9, 2.4 Hz, 1H), 7.46 (t, J = 7.7 Hz, 1H), 7.34 |
| | min (B) | (dt, J = 7.5, 4.0 Hz, 4H), 7.25 (d, J = 1.9 Hz, 1H), 7.12 (d, J = 8.1 Hz, |
| | | 1H), 6.97 (d, J = 8.9 Hz, 1H), 5.11 (dd, J = 13.0, 4.8 Hz, 1H), 4.61 |
| | | (s, 2H), 4.46 (d, J = 17.5 Hz, 1H), 4.35 (d, J = 17.5 Hz, 1H), 4.23- |
| | | 4.09 (m, 4H), 3.89 (s, 1H), 3.49-3.47 (m, 1H), 3.20-3.07 (m, |
| | | 4H), 2.90 (br d, J = 10.6 Hz, 1H), 2.76-2.68 (m, 2H), 2.66-2.57 |
| | | (m, 1H), 2.55 (DMSO), 2.49-2.37 (m, 3H), 2.33 (br d, J = 13.3 |
| | | Hz, 2H), 2.25 (br t, J = 7.6 Hz, 2H), 2.07-1.97 (m, 1H), 1.67- |
| | | 1.55 (m, 2H), 1.48-1.32 (m, 7H), 1.21 (br s, 16H). Proton |
| | | missing due to H2O suppression near 3.4. |
| Example | 1140.6 | ¹H NMR (500 MHz, DMSO-d₆) δ 10.99 (s, 1H), 8.60 (d, J = 1.1 |
| 30 | [M + H]⁺, | Hz, 1H), 8.54 (s, 1H), 8.31 (d, J = 2.4 Hz, 1H), 8.12 (s, 1H), 8.06 |
| | RT = 2.45 | (br d, J = 7.5 Hz, 1H), 7.74 (dd, J = 8.7, 2.6 Hz, 1H), 7.46 (t, J = 7.8 |
| | min (A) | Hz, 1H), 7.39-7.29 (m, 4H), 7.25 (d, J = 2.0 Hz, 1H), 7.11 (d, |
| | | J = 8.2 Hz, 1H), 6.96 (d, J = 8.8 Hz, 1H), 5.10 (dd, J = 13.4, 5.0 Hz, |
| | | 1H), 4.61 (s, 2H), 4.46 (d, J = 17.5 Hz, 1H), 4.34 (d, J = 17.3 Hz, |
| | | 1H), 4.24-4.11 (m, 4H), 3.77 (br s, 1H), 3.66-3.49 (m, 29H), |
| | | 3.45-3.29 (m, 1H), 3.26-3.07 (m, 5H), 2.99-2.80 (m, 1H), 2.78- |
| | | 2.67 (m, 2H), 2.66-2.53 (m, 4H), 2.48-2.30 (m, 5H), 2.25 (br |
| | | t, J = 7.4 Hz, 2H), 2.09-1.98 (m, 1H), 1.70-1.52 (m, 2H), 1.48- |
| | | 1.25 (m, 10H), 1.21 (m, 18H) |

### Example 31

### N-[1-[5-(3-cyano-6-ethoxy-pyrazolo[1,5-a]pyridin-4-yl)-2-pyridyl]-4-[[2-[4-[4-[2-(2,6-dioxo-3-piperidyl)-1-oxo-isoindolin-5-yl]piperazin-1-yl]butylamino]-2-oxo-ethoxy]methyl]-4-piperidyl]-2,5-difluoro-benzamide

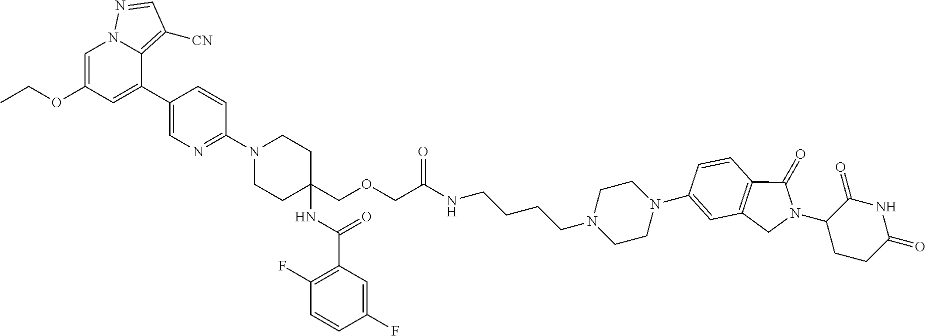 [SMILES: CCOc1cc(-c2ccc(N3CCC(COCC(=O)NCCCCN4CCN(c5ccc6c(c5)CN(C5CCC(=O)NC5=O)C6=O)CC4)(NC(=O)c4cc(F)ccc4F)CC3)nc2)c2c(C#N)cnn2c1]

### A. Tert-butyl N-(4-oxobutyl) carbamate

To a solution of tert-butyl N-(4-hydroxybutyl) carbamate (200 mg, 1.06 mmol) in DCM (10 mL) was added Dess-Martin (896.17 mg, 2.11 mmol) at 0 °C, the mixture was stirred at rt for 1 h. The reaction was purified by prep-TLC to afford tert-butyl N-(4-oxobutyl) carbamate (150 mg, 75.8% yield) as a colorless oil.

### B. Tert-butyl N-[4-[4-[2-(2,6-dioxo-3-piperidyl)-1-oxo-isoindolin-5-yl]piperazin-1-yl]butyl]carbamate

To a solution of 3-(1-oxo-5-piperazin-1-yl-isoindolin-2-yl)piperidine-2,6-dione (45 mg, 0.14 mmol) and tert-butyl N-(4-oxobutyl) carbamate (26 mg, 0.14 mmol) in DCM (10 mL) was added DIEA (0.05 mL, 0.27 mmol). The mixture was stirred at rt for 10 min. And then AcOH (0.02 mL, 0.27 mmol) was added, the mixture was stirred at rt for 0.5 h. Then NaBH(OAc)₃ (60.0 mg, 0.27 mmol) was added at 0 °C, the mixture was stirred at rt for 3 h. The reaction was concentrated and purified by Prep-HPLC to afford the desired product (60 mg, 87.6% yield) as an off-white solid. MS: m/z [M+H]⁺ 500.

### C. 3-[5-[4-(4-aminobutyl)piperazin-1-yl]-1-oxo-isoindolin-2-yl]piperidine-2,6-dione

To a solution of tert-butyl N-[4-[4-[2-(2,6-dioxo-3-piperidyl)-1-oxo-isoindolin-5-yl]piperazin-1-yl]butyl]carbamate (60 mg, 0.12 mmol) in DCM (10 mL) was added TFA (2 mL), and the mixture was stirred at rt for 4 h. It was concentrated to afford the desired product (50 mg) as an off-white solid, which was used in the next step without further purification. MS: m/z [M+H]⁺ 400.

### D. Tert-butyl 2-[[1-[5-(3-cyano-6-ethoxy-pyrazolo[1,5-a]pyridin-4-yl)-2-pyridyl]-4-[(2,5-difluorobenzoyl)amino]-4-piperidyl]methoxy]acetate

To a solution of N-[1-[5-(3-cyano-6-ethoxy-pyrazolo[1,5-a]pyridin-4-yl)-2-pyridyl]-4-(hydroxymethyl)-4-piperidyl]-2,5-difluoro-benzamide (200 mg, 0.38 mmol) in THF (5 mL) was added NaH (90.13 mg, 3.76 mmol) at 0 °C. The mixture was stirred for 0.5 h. tert-butyl 2-bromoacetate (87.9 mg, 0.45 mmol) was added at 0 °C, the reaction mixture was stirred at rt for 2 h. Water was used to quench the reaction, which was extracted with EA, combined the organic phase, dried over anhydrous sodium sulphate, filtered, and concentrated. The crude was purified by prep-TLC (DCM:MeOH=20:1) to afford the desired product (220 mg, 90.6% yield) as a yellow solid. MS: m/z [M+H]⁺ 647.

### E. 2-[[1-[5-(3-cyano-6-ethoxy-pyrazolo[1,5-a]pyridin-4-yl)-2-pyridyl]-4-[(2,5-difluorobenzoyl)amino]-4-piperidyl]methoxy]acetic acid

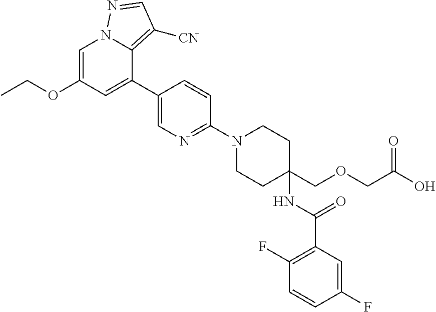 [SMILES: CCOc1cc(-c2ccc(N3CCC(COCC(=O)O)(NC(=O)c4cc(F)ccc4F)CC3)nc2)c2c(C#N)cnn2c1]

To a solution of tert-butyl 2-[[1-[5-(3-cyano-6-ethoxy-pyrazolo[1,5-a]pyridin-4-yl)-2-pyridyl]-4-[(2,5-difluorobenzoyl)amino]-4-piperidyl]methoxy]acetate (30 mg, 0.05 mmol) in DCM (2 mL) was added TFA (0.5 mL). The mixture was stirred at rt for 1 h. It was concentrated, and the crude was purified by Prep-HPLC to afford the desired product (15.1 mg, 54.5% yield) as a white solid. ¹H NMR (400 MHz, DMSO-d6) δ 12.67 (s, 1H), 8.69-8.54 (m, 2H), 8.33 (d, J=2.6 Hz, 1H), 8.17 (s, 1H), 7.76 (dd, J=8.8, 2.6 Hz, 1H), 7.47-7.22 (m, 4H), 6.99 (d, J=8.8 Hz, 1H), 4.23-4.12 (m, 4H), 4.05 (s, 2H), 3.74 (s, 2H), 3.29-3.18 (m, 2H), 2.36-2.32 (m, 2H), 1.70-1.62 (m, 2H), 1.38 (t, J=7.0 Hz, 3H); MS: m/z: [M+H]⁺ 591. Prep HPLC purification conditions: Column: Xselect CSH C18 OBD Column 30×150 mm 5 μm; Mobile Phase A: Water (0.1% FA), Mobile Phase B: ACN; Flow rate: 60 mL/min; Gradient: 21% B to 51% B in 10 min, 51% B; RT=9.8 min.

### F. N-[1-[5-(3-cyano-6-ethoxy-pyrazolo[1,5-a]pyridin-4-yl)-2-pyridyl]-4-[[2-[4-[4-[2-(2,6-dioxo-3-piperidyl)-1-oxo-isoindolin-5-yl]piperazin-1-yl]butylamino]-2-oxo-ethoxy]methyl]-4-piperidyl]-2,5-difluoro-benzamide

To a solution of 3-[5-[4-(4-aminobutyl)piperazin-1-yl]-1-oxo-isoindolin-2-yl]piperidine-2,6-dione (50 mg, 0.12 mmol), 2-[[1-[5-(3-cyano-6-ethoxy-pyrazolo[1,5-a]pyridin-4-yl)-2-pyridyl]-4-[(2,5-difluorobenzoyl)amino]-4-piperidyl]methoxy]acetic acid (40 mg, 0.07 mmol) and HATU (50 mg, 0.12 mmol) in DMF (3 mL) was added DIEA (26 mg, 0.20 mmol) at −5 °C, the mixture was stirred for 1 h and purified by Prep-HPLC to afford the titled compound (28.6 mg, 43.4% yield) as a white solid. ¹H NMR (300 MHz, DMSO-d6) δ 10.96 (s, 1H), 9.47 (brs, 1H), 8.66 (d, J=2.0 Hz, 1H), 8.58 (s, 1H), 8.34 (d, J=2.5 Hz, 1H), 8.15 (s, 1H), 7.82-7.78 (m, 1H), 7.76-7.65 (m, 1H), 7.60-7.57 (m, 1H), 7.46-7.26 (m, 4H), 7.19-7.00 (m, 3H), 5.09-5.03 (m, 1H), 4.40-4.29 (m, 1H), 4.29-4.09 (m, 5H), 4.07-3.89 (m, 5H), 3.77-3.71 (m, 3H), 3.30-3.03 (m, 10H), 2.95-2.81 (m, 1H), 2.65-2.55 (m, 1H), 2.42-2.31 (m, 3H), 2.02-1.91 (m, 1H), 1.77-1.60 (m, 4H), 1.52-1.41 (m, 2H), 1.38 (t, J=7.0 Hz, 3H); MS: m/z: [M+H]⁺ 972. Prep HPLC purification conditions: Column: Xselect CSH C18 OBD Column 30×150 mm 5 μm; Mobile Phase A: Water (0.05% TFA), Mobile Phase B: ACN; Flow rate: 60 mL/min; Gradient: 5% B to 35% B in 10 min, 35% B; RT=10.32 min.

### Example 32

### N-[1-[5-(3-cyano-6-ethoxy-pyrazolo[1,5-a]pyridin-4-yl)-2-pyridyl]-4-[2-[4-[3-[4-[2-(2,6-dioxo-3-piperidyl)-3-oxo-isoindolin-5-yl]-1-piperidyl]propanoyl]piperazin-1-yl]ethoxymethyl]-4-piperidyl]-2,5-difluoro-benzamide

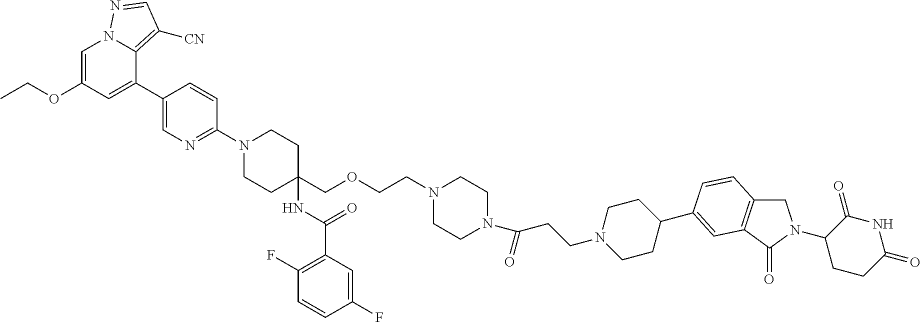 [SMILES: CCOc1cc(-c2ccc(N3CCC(COCCN4CCN(C(=O)CCN5CCC(c6ccc7c(c6)C(=O)N(C6CCC(=O)NC6=O)C7)CC5)CC4)(NC(=O)c4cc(F)ccc4F)CC3)nc2)c2c(C#N)cnn2c1]

### A. Tert-butyl 3-[4-[2-(2,6-dioxo-3-piperidyl)-3-oxo-isoindolin-5-yl]-1-piperidyl]propanoate

To a solution of 3-[1-oxo-6-(4-piperidyl) isoindolin-2-yl]piperidine-2,6-dione (50 mg, 0.15 mmol), tert-butyl prop-2-enoate (39.15 mg, 0.31 mmol) in MeCN (5 mL) was added DBU (0.07 mL, 0.46 mmol). The mixture was stirred at 70 °C for 16 h. The reaction was concentrated, and the crude was purified by reversed phase column chromatography to afford the desired product (60 mg, 86.2% yield) as a white solid. MS: m/z [M+H]⁺ 456.

### B. 3-[4-[2-(2,6-dioxo-3-piperidyl)-3-oxo-isoindolin-5-yl]-1-piperidyl]propanoic acid

To a solution of tert-butyl 3-[4-[2-(2,6-dioxo-3-piperidyl)-3-oxo-isoindolin-5-yl]-1-piperidyl]propanoate (60 mg, 0.13 mmol) in DCM (3 mL) was added TFA (1 mL). The mixture was stirred at rt for 1 h. The reaction was concentrated to afford crude product, which was used directly in the next step without further purification. MS: m/z [M+H]⁺ 400.

### C. N-[1-[5-(3-cyano-6-ethoxy-pyrazolo[1,5-a]pyridin-4-yl)-2-pyridyl]-4-(2-hydroxyethoxymethyl)-4-piperidyl]-2,5-difluoro-benzamide

To a solution of 2-[[1-[5-(3-cyano-6-ethoxy-pyrazolo[1,5-a]pyridin-4-yl)-2-pyridyl]-4-[(2,5-difluorobenzoyl)amino]-4-piperidyl]methoxy]acetic acid (600 mg, 1.02 mmol) in THF (5 mL) were added ClCO2Bu-i (167.02 mg, 1.22 mmol), NMM (102.8 mg, 1.02 mmol) at −15 °C. The mixture was stirred at −15 °C for 0.5 h, NaBH₄ (115.3 mg, 3.05 mmol) was added. The mixture was stirred for 2 h. It was concentrated purified by prep-TLC (DCM:MeOH=20:1) to afford the desired product (400 mg, 68.3% yield) as a white solid. MS: m/z [M+H]⁺ 577.

### D. N-[1-[5-(3-cyano-6-ethoxy-pyrazolo[1,5-a]pyridin-4-yl)-2-pyridyl]-4-(2-oxoethoxymethyl)-4-piperidyl]-2,5-difluoro-benzamide

To a solution of (COCl)₂ (0.11 mL, 1.39 mmol) in DCM (2 mL) was added DMSO (0.2 mL, 2.77 mmol) at −78 °C, the mixture was stirred at −78 °C for 0.5 h. N-[1-[5-(3-cyano-6-ethoxy-pyrazolo[1,5-a]pyridin-4-yl)-2-pyridyl]-4-(2-hydroxyethoxymethyl)-4-piperidyl]-2,5-difluoro-benzamide (400 mg, 0.69 mmol) in DCM (0.5 mL) was added, the mixture was stirred at −78 °C for 2 h. TEA (0.6 mL, 3.47 mmol) was added, the mixture was stirred at −78 °C for 0.5 h. Water was used to quench the reaction, extracted, dried over anhydrous sodium sulphate, filtered, concentrated to afford crude product which was used directly in the next step without further purification. MS: m/z [M+H]⁺ 575.

### E. Tert-butyl 4-[2-[[1-[5-(3-cyano-6-ethoxy-pyrazolo[1,5-a]pyridin-4-yl)-2-pyridyl]-4-[(2,5-difluorobenzoyl)amino]-4-piperidyl]methoxy]ethyl]piperazine-1-carboxylate

To a solution of N-[1-[5-(3-cyano-6-ethoxy-pyrazolo[1,5-a]pyridin-4-yl)-2-pyridyl]-4-(2-oxoethoxymethyl)-4-piperidyl]-2,5-difluoro-benzamide (30 mg, 0.05 mmol), tert-butyl piperazine-1-carboxylate (29.17 mg, 0.16 mmol), in DCM (1 mL) was added titanium (IV) isopropoxide (29.66 mg, 0.10 mmol). The mixture was stirred at rt for 1 h. NaBH(OAc)₃ (22.13 mg, 0.10 mmol) was added at 0 °C. It was stirred at rt for 6 h. The crude was purified by Prep-TLC (DCM:MeOH=20:1) to afford the desired product (28 mg, 72% yield) as a white solid. MS: m/z [M+H]⁺ 745.

### F. N-[[1-][5-(3-cyano-6-ethoxy-pyrazolo[1,5-a]pyridin-4-yl)-2-pyridyl]-4-(2-piperazin-1-ylethoxymethyl)-4-piperidyl]-2,5-difluoro-benzamide.)

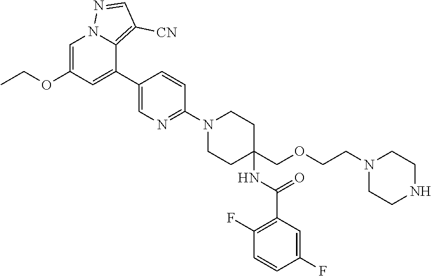 [SMILES: CCOc1cc(-c2ccc(N3CCC(COCCN4CCNCC4)(NC(=O)c4cc(F)ccc4F)CC3)nc2)c2c(C#N)cnn2c1]

To a solution of tert-butyl 4-[[2-[1-[5-(3-cyano-6-ethoxy-pyrazolo[1,5-a]pyridin-4-yl)-2-pyridyl]-4-[(2,5-difluorobenzoyl)amino]-4-piperidyl]methoxy]ethyl]piperazine-1-carboxylate (30 mg, 0.04 mmol) in DCM (3 mL) was added TFA (1 mL). The mixture was stirred at rt for 1 h. It was concentrated, and the crude was purified by prep-HPLC to afford the desired product (13.2 mg, 49.7% yield) as a white solid. ¹H NMR (400 MHz, DMSO-d6) δ 9.24 (s, 2H), 8.68 (d, J=2.5 Hz, 1H), 8.59 (s, 1H), 8.34 (d, J=2.5 Hz, 1H), 8.15 (s, 1H), 7.84 (dd, J=8.9, 2.6 Hz, 1H), 7.45-7.27 (m, 4H), 7.07 (d, J=8.9 Hz, 1H), 4.27-4.13 (m, 4H), 3.83-3.69 (m, 4H), 3.36-3.33 (m, 6H), 3.26-3.20 (m, 2H), 2.51-2.50 (m, 4H), 2.36-2.33 (m, 2H), 1.71-1.63 (m, 2H), 1.39 (t, J=6.9 Hz, 3H); MS: m/z: [M+H]⁺ 645. Prep-HPLC purification condition: Column: Xselect CSH C18 OBD Column 30×150 mm 5 μm; Mobile Phase A: Water (0.1% FA), Mobile Phase B: ACN; Flow rate: 60 mL/min; Gradient: 6% B to 36% B in 10 min, 36% B; RT=8.9 min.

### G. N-[1-[5-(3-cyano-6-ethoxy-pyrazolo[1,5-a]pyridin-4-yl)-2-pyridyl]-4-[2-[4-[3-[4-[2-(2,6-dioxo-3-piperidyl)-3-oxo-isoindolin-5-yl]-1-piperidyl]propanoyl]piperazin-1-yl]ethoxymethyl]-4-piperidyl]-2,5-difluoro-benzamide

To a solution of N-[1-[5-(3-cyano-6-ethoxy-pyrazolo[1,5-a]pyridin-4-yl)-2-pyridyl]-4-(2-piperazin-1-ylethoxymethyl)-4-piperidyl]-2,5-difluoro-benzamide (30 mg, 0.05 mmol), 3-[4-[2-(2,6-dioxo-3-piperidyl)-3-oxo-isoindolin-5-yl]-1-piperidyl]propanoic acid (18.59 mg, 0.05 mmol) in DMA (3 mL) were added HATU (26.54 mg, 0.07 mmol), DIEA (0.01 mL, 0.14 mmol). The mixture was stirred at rt for 2 h. The crude was purified by Prep-HPLC to afford the titled product (6.6 mg, 13.6% yield) as a white solid. ¹H NMR (400 MHz, DMSO-d6) δ 10.98 (s, 1H), 8.65 (d, J=2.5 Hz, 1H), 8.58 (s, 1H), 8.33 (d, J=2.5 Hz, 1H), 8.11 (s, 1H), 7.76 (dd, J=9.0, 2.5 Hz, 1H), 7.55-7.52 (m, 3H), 7.40-7.30 (m, 3H), 7.27 (d, J=2.5 Hz, 1H), 6.98 (d, J=9.0 Hz, 1H), 5.11 (dd, J=13.3, 5.1 Hz, 1H), 4.43-4.38 (m, 1H), 4.29-4.26 (m, 1H), 4.24-4.10 (m, 4H), 3.65 (s, 2H), 3.58-3.55 (m, 2H), 3.40-3.38 (m, 4H), 3.22-3.16 (m, 2H), 3.00-2.85 (m, 3H), 2.69-2.55 (m, 2H), 2.51-2.49 (m, 3H), 2.47-2.40 (m, 5H), 2.39-2.26 (m, 5H), 2.06-1.98 (m, 3H), 1.78-1.74 (m, 2H), 1.68-1.59 (m, 4H), 1.38 (t, J=7.0 Hz, 3H); Analytical LCMS ESI-MS(+) m/z 514.1 [M+2H]²⁺, RT=1.40 min, purity: 98.1% (Method Q); Prep HPLC purification conditions: Column: YMC-Actus Triart C18 E×RS, 30×150 mm, 5 μm; Mobile Phase A: Water (10 mmol/L NH₄HCO₃+0.1% NH₃·H₂O), Mobile Phase B: ACN; Flow rate: 60 mL/min; Gradient: 34% B to 64% B in 10 min, 64% B; RT=9.35 min.

### Example 33

### N-[1-[5-(3-cyano-6-ethoxy-pyrazolo[1,5-a]pyridin-4-yl)-2-pyridyl]-4-[2-[4-[3-[4-[2-(2,6-dioxo-3-piperidyl)-3-oxo-isoindolin-5-yl]piperazin-1-yl]propanoyl]piperazin-1-yl]ethoxymethyl]-4-piperidyl]-2,5-difluoro-benzamide

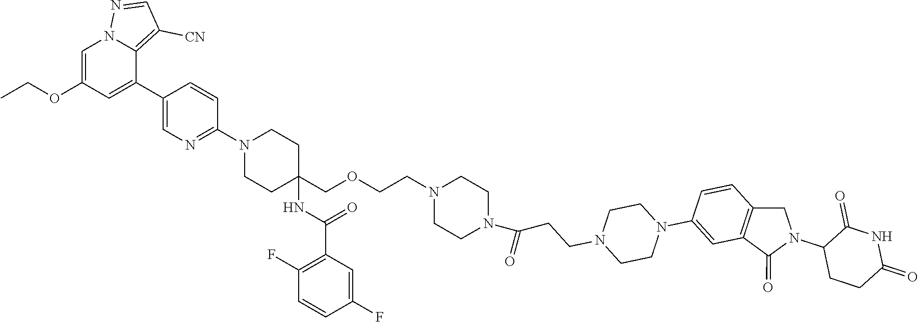 [SMILES: CCOc1cc(-c2ccc(N3CCC(COCCN4CCN(C(=O)CCN5CCN(c6ccc7c(c6)C(=O)N(C6CCC(=O)NC6=O)C7)CC5)CC4)(NC(=O)c4cc(F)ccc4F)CC3)nc2)c2c(C#N)cnn2c1]

The title compound was prepared in 21.32% overall yield as a white solid according to the preparation of Example 32 using 3-(1-oxo-6-piperazin-1-yl-isoindolin-2-yl)piperidine-2,6-dione (50 mg, 0.15 mmol), tert-butyl prop-2-enoate in STEP A. ¹H NMR (400 MHz, DMSO-d6) δ 10.98 (s, 1H), 8.65 (d, J=2.5 Hz, 1H), 8.58 (s, 1H), 8.33 (d, J=2.5 Hz, 1H), 8.11 (s, 1H), 7.76 (dd, J=8.8, 2.5 Hz, 1H), 7.42 (d, J=8.8 Hz, 1H), 7.40-7.31 (m, 3H), 7.29-7.23 (m, 2H), 7.15 (d, J=2.5 Hz, 1H), 6.98 (d, J=8.8 Hz, 1H), 5.10 (dd, J=13.3, 5.1 Hz, 1H), 4.35-4.31 (m, 1H), 4.26-4.08 (m, 5H), 3.65 (s, 2H), 3.56 (t, J=5.5 Hz, 2H), 3.43-3.39 (m, 5H), 3.26-3.10 (m, 6H), 2.96-2.87 (m, 1H), 2.64-2.53 (m, 7H), 2.46-2.26 (m, 10H), 2.01-1.97 (m, 1H), 1.70-1.55 (m, 2H), 1.38 (t, J=7.0 Hz, 3H); Analytical LCMS ESI-MS(+) m/z 514.7 [M+2H]²⁺, RT=1.38 min, purity: 98.9% (Method Q); Prep HPLC purification conditions: Column: Xselect CSH C18 OBD Column 30×150 mm 5 μm; Mobile Phase A: Water (10 mmol/L NH₄HCO₃), Mobile Phase B: ACN; Flow rate: 60 mL/min; Gradient: 23% B to 53% B in 10 min, 53% B; RT=10.7 min.

### Example 34

### N-[1-[5-(3-cyano-6-ethoxy-pyrazolo[1,5-a]pyridin-4-yl)-2-pyridyl]-4-[2-[4-[3-[4-[2-(2,6-dioxo-3-piperidyl)-1-oxo-isoindolin-5-yl]piperazin-1-yl]propanoyl]piperazin-1-yl]ethoxymethyl]-4-piperidyl]-2,5-difluoro-benzamide

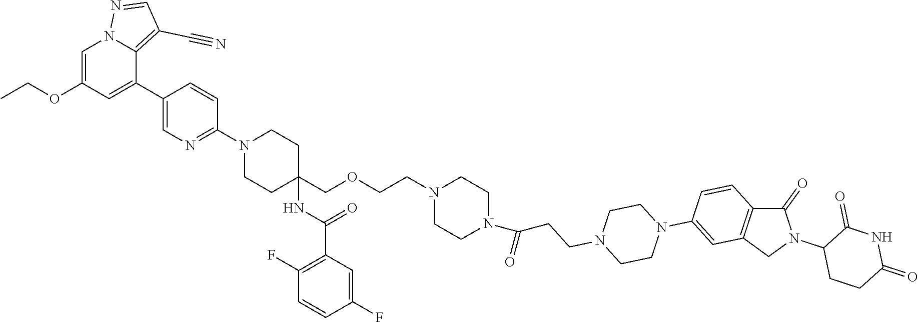 [SMILES: CCOc1cc(-c2ccc(N3CCC(COCCN4CCN(C(=O)CCN5CCN(c6ccc7c(c6)CN(C6CCC(=O)NC6=O)C7=O)CC5)CC4)(NC(=O)c4cc(F)ccc4F)CC3)nc2)c2c(C#N)cnn2c1]

The title compound was prepared in 10.81% overall yield as a white solid according to the preparation of Example 32 using 3-(1-oxo-5-piperazin-1-yl-isoindolin-2-yl)piperidine-2,6-dione in STEP A. ¹H NMR (400 MHz, DMSO-d6) δ 8.65 (d, J=2.5 Hz, 1H), 8.57 (s, 1H), 8.37-8.17 (m, 1H), 7.87 (dd, J=9.0, 2.5 Hz, 1H), 7.62 (d, J=9.0 Hz, 1H), 7.45-7.28 (m, 4H), 7.22-7.08 (m, 3H), 5.05 (dd, J=13.4, 5.1 Hz, 1H), 4.41-4.36 (m, 2H), 4.31-4.10 (m, 6H), 4.08-3.95 (m, 3H), 3.84-3.81 (m, 2H), 3.76-3.73 (m, 2H), 3.45-3.36 (m, 5H), 3.29-3.08 (m, 7H), 2.93-2.87 (m, 4H), 2.71-2.58 (m, 1H), 2.36 (d, J=7.3 Hz, 2H), 2.07-1.93 (m, 1H), 1.73-1.67 (m, 2H), 1.39 (t, J=6.9 Hz, 3H); MS: m/z [M+2H]²⁺ 514.5. Prep-HPLC purification conditions: Column: Xselect CSH C18 OBD Column 30×150 mm 5 μm; Mobile Phase A: Water (0.05% TFA), Mobile Phase B: ACN; Flow rate: 60 mL/min; Gradient: 2% B to 25% B in 15 min, 25% B; RT=16.67 min.

### Example 35

### N-[1-[5-(3-cyano-6-ethoxy-pyrazolo[1,5-a]pyridin-4-yl)-2-pyridyl]-4-[2-[4-[5-[4-[2-(2,6-dioxo-3-piperidyl)-1-oxo-isoindolin-5-yl]piperazin-1-yl]pentanoyl]piperazin-1-yl]ethoxymethyl]-4-piperidyl]-2,5-difluoro-benzamide

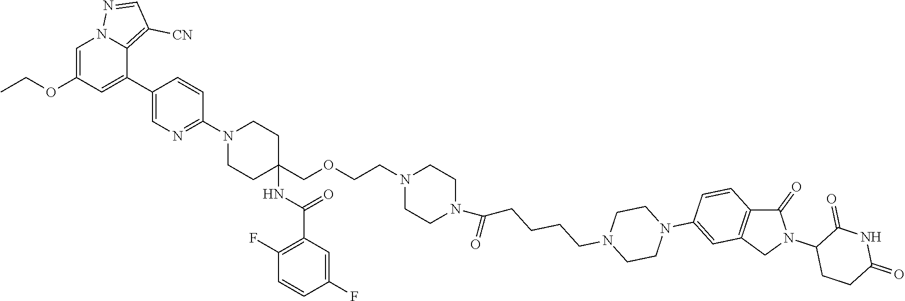 [SMILES: CCOc1cc(-c2ccc(N3CCC(COCCN4CCN(C(=O)CCCCN5CCN(c6ccc7c(c6)CN(C6CCC(=O)NC6=O)C7=O)CC5)CC4)(NC(=O)c4cc(F)ccc4F)CC3)nc2)c2c(C#N)cnn2c1]

The title compound was prepared in 29.12% overall yield as a white solid according to the preparation of Example 31 using 3-(1-oxo-5-piperazin-1-yl-isoindolin-2-yl)piperidine-2,6-dione in STEP B. ¹H NMR (400 MHz, DMSO-d6) δ 8.68 (d, J=2.5 Hz, 1H), 8.59 (s, 1H), 8.35 (d, J=2.5 Hz, 1H), 7.90 (dd, J=9.0, 2.5 Hz, 1H), 7.61 (d, J=9.0 Hz, 1H), 7.47-7.29 (m, 4H), 7.21-7.13 (m, 3H), 5.06 (dd, J=13.3, 5.1 Hz, 1H), 4.30-4.35 (m, 2H), 4.29-4.12 (m, 5H), 4.09-3.97 (m, 3H), 3.83-3.81 (m, 2H), 3.75-3.71 (m, 2H), 3.64-3.46 (m, 4H), 3.39-3.32 (m, 3H), 3.30-3.24 (m, 2H), 3.18-3.11 (m, 7H), 2.95-2.86 (m, 3H), 2.69-2.57 (m, 1H), 2.46-2.30 (m, 5H), 2.01-1.95 (m, 1H), 1.73-1.65 (m, 4H), 1.54-1.51 (m, 2H), 1.39 (t, J=6.9 Hz, 3H). Analytical LCMS ESI-MS(+) m/z 528.6 [M+2H]²⁺, RT=1.37 min, purity: 97.5% (Method Q); Prep HPLC purification conditions: Column: Xselect CSH C18 OBD Column 30×150 mm 5 μm; Mobile Phase A: Water (0.05% TFA), Mobile Phase B: ACN; Flow rate: 60 mL/min; Gradient: 2% B to 25% B in 20 min, 25% B; RT=17.38 min.

### Example 36

### N-[1-[5-(3-cyano-6-ethoxy-pyrazolo[1,5-a]pyridin-4-yl)-2-pyridyl]-4-[[2-[4-[[4-[2-(2,6-dioxo-3-piperidyl)-1-oxo-isoindolin-5-yl]piperazin-1-yl]methyl]-1-piperidyl]-2-oxo-ethoxy]methyl]-4-piperidyl]-2,5-difluoro-benzamide

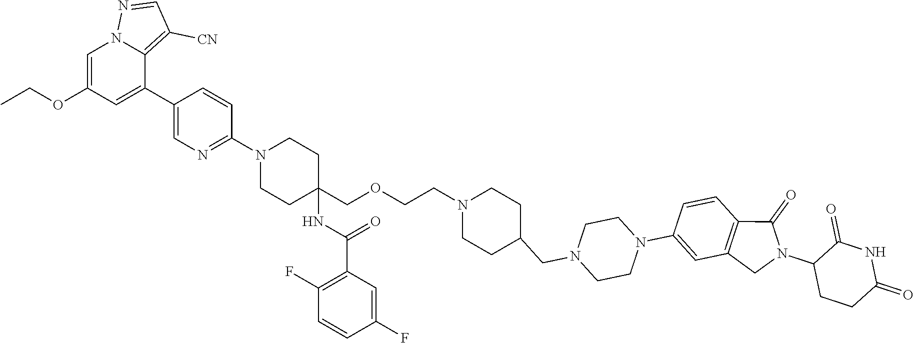 [SMILES: CCOc1cc(-c2ccc(N3CCC(COCCN4CCC(CN5CCN(c6ccc7c(c6)CN(C6CCC(=O)NC6=O)C7=O)CC5)CC4)(NC(=O)c4cc(F)ccc4F)CC3)nc2)c2c(C#N)cnn2c1]

The title compound was prepared in 11.5% overall yield as a white solid according to the preparation of Example 31 using 3-(1-oxo-5-piperazin-1-yl-isoindolin-2-yl)piperidine-2,6-dione in STEP B. ¹H NMR (300 MHz, DMSO-d6) δ 10.96 (s, 1H), 9.47 (brs, 1H), 8.66 (d, J=2.0 Hz, 1H), 8.58 (s, 1H), 8.34-8.33 (m, 1H), 8.20-8.16 (m, 1H), 7.82-7.78 (m, 1H), 7.76-7.65 (m, 1H), 7.46-7.26 (m, 4H), 7.19-7.00 (m, 3H), 5.09-5.03 (m, 1H), 4.42-4.24 (m, 3H), 4.24-4.02 (m, 7H), 4.02-3.94 (m, 2H), 3.74-3.68 (m, 2H), 3.33-3.11 (m, 7H), 3.11-3.01 (m, 3H), 2.69-2.60 (m, 1H), 2.38-2.28 (m, 4H), 2.14-2.03 (m, 1H), 2.03-1.92 (m, 1H), 1.82-1.64 (m, 4H), 1.38 (t, J=7.0 Hz, 3H), 1.23-1.06 (m, 3H); Analytical LCMS ESI-MS(+) m/z 500.1 [M+2H]²⁺, RT=1.45 min, purity: 96.9% (Method Q); Prep HPLC purification conditions: Column: XBridge Prep Phenyl OBD Column, 19×250 mm, 5 μm; Mobile Phase A: Water (10 mmol/L NH₄HCO_{3+0.1}% NH₃·H₂O), Mobile Phase B: ACN; Flow rate: 25 mL/min; Gradient: 30% B to 40% B in 8 min, 40% B; RT=7.75 min.

### Example 37

### N-[1-[5-(3-cyano-6-ethoxy-pyrazolo[1,5-a]pyridin-4-yl)-2-pyridyl]-4-[2-[4-[3-[4-[2-(2,6-dioxo-3-piperidyl)-3-oxo-isoindolin-5-yl]-1-piperidyl]propyl]piperazin-1-yl]ethoxymethyl]-4-piperidyl]-2,5-difluoro-benzamide

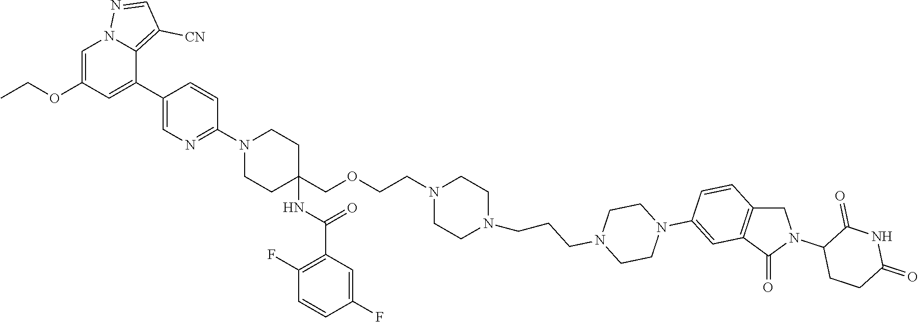 [SMILES: CCOc1cc(-c2ccc(N3CCC(COCCN4CCN(CCCN5CCN(c6ccc7c(c6)C(=O)N(C6CCC(=O)NC6=O)C7)CC5)CC4)(NC(=O)c4cc(F)ccc4F)CC3)nc2)c2c(C#N)cnn2c1]

### A. Benzyl 4-[2-(1,3-dioxolan-2-yl)ethyl]piperazine-1-carboxylate

To a solution of benzyl piperazine-1-carboxylate (1000 mg, 4.54 mmol) and 2-(2-bromoethyl)-1,3-dioxolane (822 mg, 4.54 mmol) in DMF (20 mL) was added DIEA (1757 mg, 13.62 mmol), the mixture solution was stirred at 50 °C for overnight. The reaction was diluted with water, extracted with EtOAc, washed with brine, dried, concentrated to afford the desired product (1200 mg, 81.2% yield), which was used in the next step without further purification. MS: m/z [M+H]⁺ 321.

### B. 1-[2-(1,3-dioxolan-2-yl)ethyl]piperazine

The mixture solution of benzyl 4-[2-(1,3-dioxolan-2-yl)ethyl]piperazine-1-carboxylate (1100 mg, 3.43 mmol) in Methanol (20 mL) was purged with N₂ (3×), and then Pd/C (365 mg, 3.43 mmol) was added, H₂ was introduced to above mixture and the resulting mixture was stirred for overnight. Upon completion, the reaction was filtered, and the filtrate was concentrated to afford 1-[2-(1,3-dioxolan-2-yl)ethyl]piperazine (500 mg, 76.9% yield) as an off white solid, which was used directly in the next step without further purification. ¹H NMR (300 MHz, Chloroform-d) δ 4.99-4.87 (m, 1H), 4.05-3.77 (m, 4H), 3.13-2.89 (m, 4H), 2.57-2.43 (m, 6H), 1.94-1.79 (m, 2H).

### C. N-[1-[5-(3-cyano-6-ethoxy-pyrazolo[1,5-a]pyridin-4-yl)-2-pyridyl]-4-[2-[4-[2-(1,3-dioxolan-2-yl)ethyl]piperazin-1-yl]ethoxymethyl]-4-piperidyl]-2,5-difluoro-benzamide

The mixture solution of 1-[2-(1,3-dioxolan-2-yl)ethyl]piperazine (51.86 mg, 0.2800 mmol) and N-[1-[5-(3-cyano-6-ethoxy-pyrazolo[1,5-a]pyridin-4-yl)-2-pyridyl]-4-(2-oxoethoxymethyl)-4-piperidyl]-2,5-difluoro-benzamide (160 mg, 0.2800 mmol) in DCM (40 mL) were stirred at rt for 0.5 h. The reaction was cooled down to 0 °C, then NaBH(OAc)₃ (59.02 mg, 0.28 mmol) was added, and the mixture was stirred at rt for overnight. The reaction was purified by Prep-TLC to afford the desired product (50 mg, 24.1% yield) as an off white solid. MS: m/z [M+H]⁺ 745.

### D. N-[1-[5-(3-cyano-6-ethoxy-pyrazolo[1,5-a]pyridin-4-yl)-2-pyridyl]-4-[2-[4-(3-oxopropyl)piperazin-1-yl]ethoxymethyl]-4-piperidyl]-2,5-difluoro-benzamide

To the mixture solution N-[1-[5-(3-cyano-6-ethoxy-pyrazolo[1,5-a]pyridin-4-yl)-2-pyridyl]-4-[2-[4-[2-(1,3-dioxolan-2-yl)ethyl]piperazin-1-yl]ethoxymethyl]-4-piperidyl]-2,5-difluoro-benzamide (60 mg, 0.08 mmol) in acetone (3 mL) was added water (3 mL), the reaction was cooled down to 0 °C, then HCl (3 mL, 39.18 mmol) was added, the reaction was stirred at rt for 4 h. The reaction was diluted with water (20 mL) and extracted with EA, the water layer was cooled down to −10 °C, pH was adjusted to 7-8 with sat. NaHCO₃ solution, and extracted with EA, the organic layer was washed with brine, then dried, concentrated at 10 °C to afford the desired product (50 mg, 88.6% yield) as an off-white semi-solid, which was used directly in the next step without further purification. MS: m/z [M+H]⁺ 701.

### E. N-[1-[5-(3-cyano-6-ethoxy-pyrazolo[1,5-a]pyridin-4-yl)-2-pyridyl]-4-[2-[4-[3-[4-[2-(2,6-dioxo-3-piperidyl)-3-oxo-isoindolin-5-yl]-1-piperidyl]propyl]piperazin-1-yl]ethoxymethyl]-4-piperidyl]-2,5-difluoro-benzamide

To a solution of N-[1-[5-(3-cyano-6-ethoxy-pyrazolo[1,5-a]pyridin-4-yl)-2-pyridyl]-4-[2-[4-(3-oxopropyl)piperazin-1-yl]ethoxymethyl]-4-piperidyl]-2,5-difluoro-benzamide (45 mg, 0.06 mmol) and 3-[1-oxo-6-(4-piperidyl) isoindolin-2-yl]piperidine-2,6-dione (25.23 mg, 0.08 mmol) in DCM (6 mL) was added DIEA (0.01 mL, 0.10 mmol), the mixture was stirred at rt for 0.5 h, then cooled down to 0 °C, NaBH(OAc)₃ (27.22 mg, 0.13 mmol) was added, the reaction was concentrated and the residue was purified by Prep-HPLC to afford the titled compound (29.9 mg, 44.3% yield) as a white solid. ¹H NMR (300 MHz, DMSO-d6) δ 10.93 (brs, 1H), 8.65 (d, J=2.1 Hz, 1H), 8.58 (s, 1H), 8.33 (d, J=2.6 Hz, 1H), 8.09 (s, 1H), 7.76 (dd, J=8.9, 2.6 Hz, 1H), 7.58-7.49 (m, 3H), 7.37-7.33 (m, 3H), 7.27 (d, J=2.1 Hz, 1H), 6.98 (d, J=8.9 Hz, 1H), 5.14-5.08 (m, 1H), 4.44-4.38 (m, 1H), 4.30-4.12 (m, 5H), 3.65 (s, 2H), 3.55-3.52 (m, 2H), 3.24-3.15 (m, 2H), 2.95-2.86 (m, 3H), 2.62-2.56 (m, 2H), 2.47-2.31 (m, 6H), 2.30-2.25 (m, 10H), 2.04-1.89 (m, 3H), 1.81-1.51 (m, 8H), 1.38 (t, J=6.9 Hz, 3H), 1.26-1.22 (m, 1H); Analytical LCMS ESI-MS(+) m/z 507.1 [M+2H]²⁺, RT=1.51 min, purity: 96.7% (Method Q); Prep HPLC purification conditions: Column: XBridge Prep OBD C18 Column, 30×150 mm, 5 μm; Mobile Phase A: Water (10 mmol/L NH₄HCO_{3+0.1}% NH₃·H₂O), Mobile Phase B: ACN; Flow rate: 60 mL/min; Gradient: 50% B to 65% B in 8 min, 65% B; RT=6.8 min.

### Example 38

### N-[1-[5-(3-cyano-6-ethoxy-pyrazolo[1,5-a]pyridin-4-yl)-2-pyridyl]-4-[[2-[6-[4-[2-(2,6-dioxo-3-piperidyl)-1-oxo-isoindolin-5-yl]piperazin-1-yl]hexylamino]-2-oxo-ethoxy]methyl]-4-piperidyl]-2,5-difluoro-benzamide

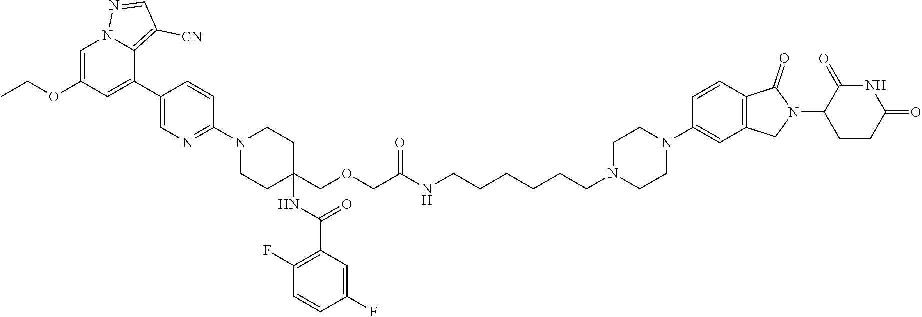 [SMILES: CCOc1cc(-c2ccc(N3CCC(COCC(=O)NCCCCCCN4CCN(c5ccc6c(c5)CN(C5CCC(=O)NC5=O)C6=O)CC4)(NC(=O)c4cc(F)ccc4F)CC3)nc2)c2c(C#N)cnn2c1]

The title compound was prepared in 10.1% overall yield as a white solid according to the preparation of Example 31 using 33-(1-oxo-5-piperazin-1-yl-isoindolin-2-yl) piperidine-2,6-dione in STEP A. ¹H NMR (400 MHz, DMSO-d6) δ 10.96 (s, 1H), 9.67 (s, 1H), 8.66 (d, J=2.1 Hz, 1H), 8.58 (s, 1H), 8.34 (d, J=2.6 Hz, 1H), 8.15 (s, 1H), 7.80 (dd, J=8.9, 2.6 Hz, 1H), 7.64-7.55 (m, 2H), 7.42-7.32 (m, 3H), 7.31-7.24 (m, 1H), 7.19-7.11 (m, 2H), 7.07-6.98 (m, 1H), 5.06 (dd, J=13.3, 5.1 Hz, 1H), 4.40-4.31 (m, 1H), 4.27-4.11 (m, 5H), 4.06-3.97 (m, 2H), 3.93-3.87 (m, 2H), 3.74-3.69 (m, 3H), 3.29-3.18 (m, 2H), 3.17-3.05 (m, 8H), 2.96-2.84 (m, 1H), 2.64-2.55 (m, 2H), 2.46-2.34 (m, 3H), 2.02-1.93 (m, 1H), 1.73-1.61 (m, 4H), 1.44-1.34 (m, 5H), 1.32-1.21 (m, 4H); MS: m/z: [M+H]⁺ 1000. Prep HPLC purification conditions: Column: XBridge Prep Phenyl OBD Column, 19×250 mm, 5 μm; Mobile Phase A: Water (0.05% TFA), Mobile Phase B: ACN; Flow rate: 25 mL/min; Gradient: 32% B to 55% B in 8 min, 55% B; RT=5.65 min.

### Example 39

### N-(1-(5-(3-cyano-6-ethoxypyrazolo[1,5-a]pyridin-4-yl)pyridin-2-yl)-4-((4-(3-(4-(3-(2,4-dioxotetrahydropyrimidin-1(2H)-yl)-1-methyl-1H-indazol-6-yl)piperazin-1-yl)propanoyl)piperazin-1-yl)methyl)piperidin-4-yl)-2,5-difluorobenzamide

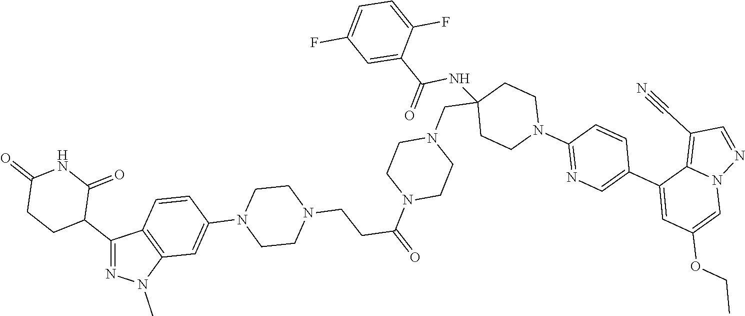 [SMILES: CCOc1cc(-c2ccc(N3CCC(CN4CCN(C(=O)CCN5CCN(c6ccc7c(C8CCC(=O)NC8=O)nn(C)c7c6)CC5)CC4)(NC(=O)c4cc(F)ccc4F)CC3)nc2)c2c(C#N)cnn2c1]

### A. 3-((6-bromo-1-methyl-1H-indazol-3-yl)amino)propanoic acid

To a solution of 6-bromo-1-methyl-indazol-3-amine (15 g, 66.35 mmol), acrylic acid (4.78 g, 66.35 mmol) in water (3 mL) was added acetic acid (2 mL). The mixture was stirred at 105 °C for 6 h. After cooling to rt, the reaction was partitioned between 1M NaOH and MBTE which dissolved all visible solid. The aqueous layer was separated and acidified with 6N HCl, concentrated, the crude was purified by reverse column chromatography to afford the desired product (7.5 g, 37.9% yield) as an off-white solid. MS: m/z [M+H]⁺ 298.

### B. 1-(6-bromo-1-methyl-1H-indazol-3-yl)dihydropyrimidine-2,4(1H,3H)-dione

To a solution of 3-[(6-bromo-1-methyl-indazol-3-yl)amino]propanoic acid (100 mg, 0.34 mmol) in acetic acid (2 mL) was added urea (72.52 mg, 1.21 mmol). The mixture was stirred at 120 °C for 16 h. The crude was purified by Prep-TLC (DCM:MeOH=20:1) to afford the desired product (30 mg, 27.7% yield) as a white solid. MS: m/z [M+H]⁺ 323.

### C. Tert-butyl 4-(3-(2,4-dioxotetrahydropyrimidin-1(2H)-yl)-1-methyl-1H-indazol-6-yl)piperazine-1-carboxylate

To 5 mL microwave vial, 1-(6-bromo-1-methyl-indazol-3-yl) hexahydropyrimidine-2,4-dione (100 mg, 0.31 mmol), tert-butyl piperazine-1-carboxylate (57.64 mg, 0.31 mmol), Ruphos Pd G3 (28.86 mg, 0.03 mmol), Ruphos (28.88 mg, 0.06 mmol) and Cs₂CO₃ (301.72 mg, 0.93 mmol) were suspended in 1,4-Dioxane (1 mL). The reaction was placed under vacuum, sonicated, and backfilled with nitrogen. The mixture was stirred at 100 °C for 16 h. It was concentrated and purified by prep-TLC (DCM:MeOH=20:1) to afford the desired product (45 mg, 33.9% yield) as a yellow solid. MS: m/z [M+H]⁺ 429.

### D. 1-(1-methyl-6-(piperazin-1-yl)-1H-indazol-3-yl)dihydropyrimidine-2,4(1H,3H)-dione

To a solution of tert-butyl 4-[3-(2,4-dioxohexahydropyrimidin-1-yl)-1-methyl-indazol-6-yl]piperazine-1-carboxylate (150 mg, 0.35 mmol) in DCM (3 mL) was added TFA (1 mL). The mixture was stirred at rt for 2 h. The reaction was concentrated to afford crude product which was used directly in the next step without further purification. MS: m/z [M+H]⁺ 329.

### E. Tert-butyl 3-(4-(3-(2,4-dioxotetrahydropyrimidin-1(2H)-yl)-1-methyl-1H-indazol-6-yl)piperazin-1-yl)propanoate

To a solution of 1-(1-methyl-6-piperazin-1-yl-indazol-3-yl) hexahydropyrimidine-2,4-dione (50 mg, 0.1500 mmol), tert-butyl prop-2-enoate (39.03 mg, 0.30 mmol) in MeCN (5 mL) was added DBU (69.54 mg, 0.46 mmol). The mixture was stirred at 40 °C for 16 h. It was concentrated and purified by reversed column chromatography to afford the desired product (60 mg, 86.3% yield) as a yellow oil. MS: m/z [M+H]⁺ 457.

### F. 3-(4-(3-(2,4-dioxotetrahydropyrimidin-1(2H)-yl)-1-methyl-1H-indazol-6-yl)piperazin-1-yl)propanoic acid

To a solution of tert-butyl 3-[4-[3-(2,4-dioxohexahydropyrimidin-1-yl)-1-methyl-indazol-6-yl]piperazin-1-yl]propanoate (60 mg, 0.13 mmol) in DCM (3 mL) was added TFA (1 mL). The mixture was stirred at rt for 2 h. It was concentrated to afford the crude product which was used directly in the next step without further purification. MS: m/z [M+H]⁺ 401.

### G. N-(1-(5-(3-cyano-6-ethoxypyrazolo[1,5-a]pyridin-4-yl)pyridin-2-yl)-4-((4-(3-(4-(3-(2,4-dioxotetrahydropyrimidin-1(2H)-yl)-1-methyl-1H-indazol-6-yl)piperazin-1-yl)propanoyl)piperazin-1-yl)methyl)piperidin-4-yl)-2,5-difluorobenzamide

To a solution of N-[1-[5-(3-cyano-6-ethoxy-pyrazolo[1,5-a]pyridin-4-yl)-2-pyridyl]-4-(piperazin-1-ylmethyl)-4-piperidyl]-2,5-difluoro-benzamide (35 mg, 0.06 mmol), 3-[4-[3-(2,4-dioxohexahydropyrimidin-1-yl)-1-methyl-indazol-6-yl]piperazin-1-yl]propanoic acid (23.33 mg, 0.06 mmol) in DMA (5 mL) were added HATU (33.23 mg, 0.09 mmol), and DIEA (0.01 mL, 0.17 mmol). The mixture was stirred at rt for 2 h. The crude was purified by Prep-HPLC to afford the titled compound (19.2 mg, 33.2% yield) as a white solid. ¹H NMR (400 MHz, DMSO-d6) δ 10.52 (s, 1H), 8.67-8.56 (m, 2H), 8.33 (d, J=2.6 Hz, 1H), 8.17 (s, 1H), 7.76 (dd, J=9.0, 2.6 Hz, 1H), 7.45 (d, J=9.0 Hz, 1H), 7.39-7.29 (m, 3H), 7.28-7.27 (m, 1H), 7.01-6.89 (m, 2H), 6.82-6.81 (m, 1H), 4.22-4.13 (m, 4H), 3.91-3.88 (m, 5H), 3.50-3.38 (m, 4H), 3.28-3.09 (m, 6H), 2.82-2.68 (m, 5H), 2.64-2.52 (m, 9H), 2.48-2.44 (m, 2H), 2.38-2.29 (m, 2H), 1.64-1.58 (m, 2H), 1.38 (t, J=6.9 Hz, 3H); Analytical LCMS ESI-MS(+) m/z 983.6 [M+H]⁺, RT=1.84 min, purity: 96.4% (Method P); Prep-HPLC purification condition: Column: XBridge Prep OBD C18 Column, 30×150 mm, 5 μm; Mobile Phase A: Water (10 mmol/L NH₄HCO_{3+0.1}% NH₃·H₂O), Mobile Phase B: ACN; Flow rate: 60 mL/min; Gradient: 40% B to 50% B in 9 min, 50% B; RT=8.2 min.

### Example 40

### N-(1-(5-(3-cyano-6-ethoxypyrazolo[1,5-a]pyridin-4-yl)pyridin-2-yl)-4-((4-(5-(4-(3-(2,4-dioxotetrahydropyrimidin-1(2H)-yl)-1-methyl-1H-indazol-6-yl)piperazin-1-yl)pentanoyl)piperazin-1-yl)methyl)piperidin-4-yl)-2,5-difluorobenzamide

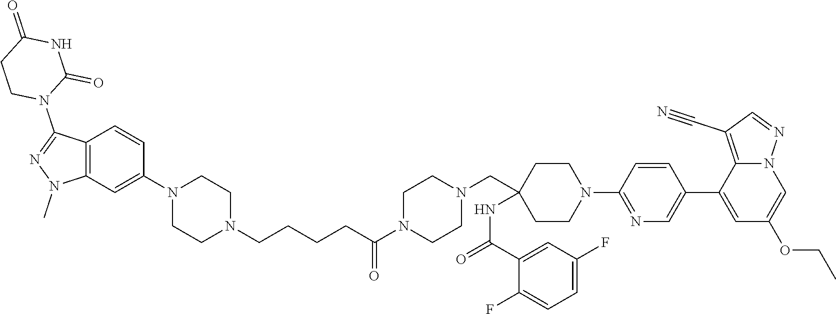 [SMILES: CCOc1cc(-c2ccc(N3CCC(CN4CCN(C(=O)CCCCN5CCN(c6ccc7c(N8CCC(=O)NC8=O)nn(C)c7c6)CC5)CC4)(NC(=O)c4cc(F)ccc4F)CC3)nc2)c2c(C#N)cnn2c1]

### A. Tert-butyl 5-(4-(3-(2,4-dioxotetrahydropyrimidin-1(2H)-yl)-1-methyl-1H-indazol-6-yl)piperazin-1-yl)pentanoate

To a solution of 1-(1-methyl-6-piperazin-1-yl-indazol-3-yl) hexahydropyrimidine-2,4-dione (50 mg, 0.15 mmol), tert-butyl 5-oxopentanoate (52.45 mg, 0.30 mmol) in DCM (5 mL) were added DIEA (39.28 mg, 0.30 mmol) and AcOH (18.29 mg, 0.30 mmol). The mixture was stirred at rt for 1 h. NaBH(OAc)₃ (64.54 mg, 0.30 mmol) was added at 0 °C, the mixture was stirred at rt for another 2 h. The solvent was evaporated to afford the crude, which was purified by reversed column chromatography to afford the desired product (43 mg, 58.3% yield) as a yellow oil. MS: m/z [M+H]⁺ 485.

### B. 5-(4-(3-(2,4-dioxotetrahydropyrimidin-1(2H)-yl)-1-methyl-1H-indazol-6-yl)piperazin-1-yl)pentanoic acid

To a solution of tert-butyl 5-[4-[3-(2,4-dioxohexahydropyrimidin-1-yl)-1-methyl-indazol-6-yl]piperazin-1-yl]pentanoate (43 mg, 0.09 mmol) in DCM (2 mL) was added TFA (1 mL), the mixture was stirred at rt for 2 h and concentrated to afford the crude product which was used directly in the next step without further purification. MS: m/z [M+H]⁺ 429.

### C. N-(1-(5-(3-cyano-6-ethoxypyrazolo[1,5-a]pyridin-4-yl)pyridin-2-yl)-4-((4-(5-(4-(3-(2,4-dioxotetrahydropyrimidin-1(2H)-yl)-1-methyl-1H-indazol-6-yl)piperazin-1-yl)pentanoyl)piperazin-1-yl)methyl)piperidin-4-yl)-2,5-difluorobenzamide

To a solution of N-[1-[5-(3-cyano-6-ethoxy-pyrazolo[1,5-a]pyridin-4-yl)-2-pyridyl]-4-(piperazin-1-ylmethyl)-4-piperidyl]-2,5-difluoro-benzamide (35 mg, 0.06 mmol), 5-[4-[3-(2,4-dioxohexahydropyrimidin-1-yl)-1-methyl-indazol-6-yl]piperazin-1-yl]pentanoic acid (24.97 mg, 0.06 mmol) in DMA (5 mL) were added HATU (33.23 mg, 0.09 mmol), DIEA (19.93 mg, 0.17 mmol). The mixture was stirred at rt for 2 h. The crude was purified by prep-HPLC to afford the titled compound (18.8 mg, 31.5% yield) as a white solid. ¹H NMR (400 MHz, DMSO-d6) δ 10.52 (s, 1H), 8.65 (d, J=2.5 Hz, 1H), 8.58 (s, 1H), 8.33 (d, J=2.5 Hz, 1H), 8.16 (s, 1H), 7.75 (dd, J=9.0, 2.5 Hz, 1H), 7.45 (d, J=9.0 Hz, 1H), 7.38-7.29 (m, 3H), 7.27 (d, J=2.5 Hz, 1H), 6.99-6.89 (m, 2H), 6.82-6.80 (m, 1H), 4.20-4.13 (m, 4H), 3.91-3.83 (m, 5H), 3.43-3.40 (m, 4H), 3.33-3.31 (m, 1H), 3.25-3.09 (m, 6H), 2.79-2.67 (m, 4H), 2.52-2.50 (m, 4H), 2.48-2.46 (m, 3H), 2.36-2.29 (m, 6H), 1.67-1.43 (m, 6H), 1.38 (t, J=6.9 Hz, 3H); Analytical LCMS ESI-MS(+) m/z 1011.4 [M+H]⁺, RT=1.84 min, purity: 97.4% (Method P); Prep HPLC purification conditions: Column: XBridge Prep OBD C18 Column, 30×150 mm, 5 μm; Mobile Phase A: Water (10 mmol/L NH₄HCO_{3+0.1}% NH₃·H₂O), Mobile Phase B: ACN; Flow rate: 60 mL/min; Gradient: 48% B to 58% B in 9 min, 58% B; RT=8.32 min.

### Example 41

### N-(1-(5-(3-cyano-6-ethoxypyrazolo[1,5-a]pyridin-4-yl)pyridin-2-yl)-4-((4-(7-(4-(3-(2,4-dioxotetrahydropyrimidin-1(2H)-yl)-1-methyl-1H-indazol-6-yl)piperazin-1-yl)heptanoyl)piperazin-1-yl)methyl)piperidin-4-yl)-2,5-difluorobenzamide

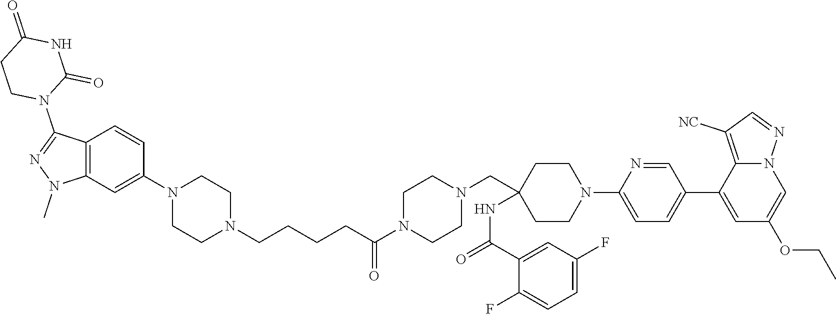 [SMILES: CCOc1cc(-c2ccc(N3CCC(CN4CCN(C(=O)CCCCN5CCN(c6ccc7c(N8CCC(=O)NC8=O)nn(C)c7c6)CC5)CC4)(NC(=O)c4cc(F)ccc4F)CC3)nc2)c2c(C#N)cnn2c1]

### A. Tert-butyl 7-(4-(3-(2,4-dioxotetrahydropyrimidin-1(2H)-yl)-1-methyl-1H-indazol-6-yl)piperazin-1-yl)heptanoate

To a solution of 1-(1-methyl-6-piperazin-1-yl-indazol-3-yl) hexahydropyrimidine-2,4-dione (60 mg, 0.18 mmol), tert-butyl 7-oxoheptanoate (73.19 mg, 0.37 mmol) in DCM (1 mL) was added DIEA (47.14 mg, 0.37 mmol) and AcOH (0.02 mL, 0.37 mmol). It was stirred at rt for 1 h, and NaBH(OAc)₃ (77.45 mg, 0.37 mmol) was added at 0 °C. The mixture was stirred at rt for another 2 h and purified by reversed column chromatography to afford the desired product (65 mg, 69.4% yield) as a yellow oil. MS: m/z [M+H]⁺ 513.

### B. 7-(4-(3-(2,4-dioxotetrahydropyrimidin-1(2H)-yl)-1-methyl-1H-indazol-6-yl)piperazin-1-yl)heptanoic acid

To a solution of tert-butyl 7-[4-[3-(2,4-dioxohexahydropyrimidin-1-yl)-1-methyl-indazol-6-yl]piperazin-1-yl]heptanoate (60 mg, 0.12 mmol) in DCM (1 mL) was added TFA (0.5 mL). The mixture was stirred at rt for 1 h. It was concentrated to afford the crude which was used directly in the next step without further purification. MS: m/z [M+H]⁺ 457.

### C. N-(1-(5-(3-cyano-6-ethoxypyrazolo[1,5-a]pyridin-4-yl)pyridin-2-yl)-4-((4-(7-(4-(3-(2,4-dioxotetrahydropyrimidin-1(2H)-yl)-1-methyl-1H-indazol-6-yl)piperazin-1-yl)heptanoyl)piperazin-1-yl)methyl)piperidin-4-yl)-2,5-difluorobenzamide

To a solution of N-[1-[5-(3-cyano-6-ethoxy-pyrazolo[1,5-a]pyridin-4-yl)-2-pyridyl]-4-(piperazin-1-ylmethyl)-4-piperidyl]-2,5-difluoro-benzamide (35 mg, 0.06 mmol), 7-[4-[3-(2,4-dioxohexahydropyrimidin-1-yl)-1-methyl-indazol-6-yl]piperazin-1-yl]heptanoic acid (26.6 mg, 0.06 mmol) in DMA (5 mL) were added HATU (33.23 mg, 0.09 mmol) and DIEA (0.01 mL, 0.17 mmol). The mixture was stirred at rt for 2 h. The crude was purified by Prep-HPLC to afford the titled compound (27.4 mg, 43.5% yield) as a white solid. ¹H NMR (400 MHz, DMSO-d6) δ 10.51 (s, 1H), 8.65 (d, J=2.5 Hz, 1H), 8.57 (s, 1H), 8.32 (d, J=2.5 Hz, 1H), 8.14 (s, 1H), 7.75 (dd, J=8.8, 2.5 Hz, 1H), 7.45 (d, J=8.8 Hz, 1H), 7.37-7.32 (m, 3H), 7.27 (d, J=2.5 Hz, 1H), 7.00-6.88 (m, 2H), 6.81 (s, 1H), 4.26-4.10 (m, 4H), 3.90-3.84 (m, 5H), 3.42-3.39 (m, 4H), 3.32-3.30 (m, 1H), 3.24-3.11 (m, 6H), 2.78-2.67 (m, 4H), 2.52-2.51 (m, 4H), 2.48-2.45 (m, 3H), 2.34-2.25 (m, 6H), 1.65-1.56 (m, 2H), 1.50-1.43 (m, 4H), 1.38 (t, J=6.9 Hz, 3H), 1.34-1.24 (m, 4H); Analytical LCMS ESI-MS(+) m/z 1039.6 [M+H]⁺, RT=1.73 min, purity: 97.3% (Method P); Prep HPLC purification conditions: Column: XBridge Prep OBD C18 Column, 30×150 mm, 5 μm; Mobile Phase A: Water (10 mmol/L NH₄HCO_{3+0.1}% NH₃·H₂O), Mobile Phase B: ACN; Flow rate: 60 mL/min; Gradient: 46% B to 60% B in 8 min, 60% B; RT=8.3 min.

### Example 42

### N-(1-(5-(3-cyano-6-ethoxypyrazolo[1,5-a]pyridin-4-yl)pyridin-2-yl)-4-((4-(6-(4-(3-(2,4-dioxotetrahydropyrimidin-1(2H)-yl)-1-methyl-1H-indazol-6-yl)piperidin-1-yl)hexanoyl)piperazin-1-yl)methyl)piperidin-4-yl)-2,5-difluorobenzamide

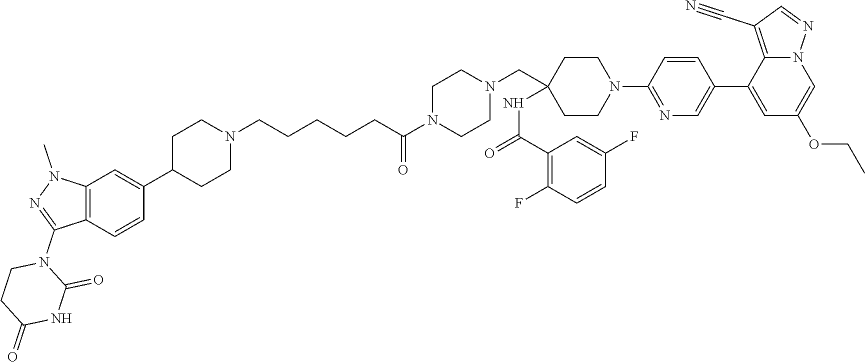 [SMILES: CCOc1cc(-c2ccc(N3CCC(CN4CCN(C(=O)CCCCCN5CCC(c6ccc7c(N8CCC(=O)NC8=O)nn(C)c7c6)CC5)CC4)(NC(=O)c4cc(F)ccc4F)CC3)nc2)c2c(C#N)cnn2c1]

### A. Tert-butyl 6-oxohexanoate

To a solution of tert-butyl 6-hydroxyhexanoate (500 mg, 2.66 mmol) in DCM (10 mL) was added Dess-Martin Periodinane (1351 mg, 3.19 mmol) at 0 °C. The mixture was stirred at rt for 2 h. The reaction was concentrated, which was purified by Prep-TLC (PE:EA=2:1) to afford product tert-butyl 6-oxohexanoate (120 mg, 24.3% yield) as a yellow oil.

### B. Tert-butyl 6-(4-(3-(2,4-dioxotetrahydropyrimidin-1(2H)-yl)-1-methyl-1H-indazol-6-yl)piperidin-1-yl) hexanoate

To a solution of 1-[1-methyl-6-(4-piperidyl) indazol-3-yl]hexahydropyrimidine-2,4-dione (60 mg, 0.18 mmol), tert-butyl 6-oxohexanoate (68.27 mg, 0.37 mmol) in DCM (10 mL) were added DIEA (0.03 mL, 0.37 mmol) and AcOH (0.02 mL, 0.37 mmol). The mixture was stirred at rt for 1 h. NaBH(OAc)₃ (77.69 mg, 0.37 mmol) was added under 0 °C. The mixture was stirred at rt for another 2 h. It was concentrated and purified by reversed column chromatography to afford the desired product (70 mg, 76.8% yield) as a yellow oil. MS: m/z [M+H]⁺ 498.

### C. 6-(4-(3-(2,4-dioxotetrahydropyrimidin-1(2H)-yl)-1-methyl-1H-indazol-6-yl)piperidin-1-yl) hexanoic acid

To a solution of tert-butyl 6-[4-[3-(2,4-dioxohexahydropyrimidin-1-yl)-1-methyl-indazol-6-yl]-1-piperidyl]hexanoate (60 mg, 0.12 mmol) in DCM (3 mL) was added TFA (1 mL). The mixture was stirred at rt for 1 h. The reaction was concentrated to afford crude product and used directly in the next step without further purification. MS: m/z [M+H]⁺ 442.

### D. N-(1-(5-(3-cyano-6-ethoxypyrazolo[1,5-a]pyridin-4-yl)pyridin-2-yl)-4-((4-(6-(4-(3-(2,4-dioxotetrahydropyrimidin-1(2H)-yl)-1-methyl-1H-indazol-6-yl)piperidin-1-yl)hexanoyl)piperazin-1-yl)methyl)piperidin-4-yl)-2,5-difluorobenzamide

To a solution of N-[1-[5-(3-cyano-6-ethoxy-pyrazolo[1,5-a]pyridin-4-yl)-2-pyridyl]-4-(piperazin-1-ylmethyl)-4-piperidyl]-2,5-difluoro-benzamide (35 mg, 0.06 mmol), 6-[4-[3-(2,4-dioxohexahydropyrimidin-1-yl)-1-methyl-indazol-6-yl]-1-piperidyl]hexanoic acid (25.73 mg, 0.06 mmol) in DMA (5 mL) were added HATU (22.16 mg, 0.06 mmol), and DIEA (6.64 mg, 0.06 mmol). The mixture was stirred at rt for 2 h. The crude was purified by prep-HPLC to afford the titled compound (19.5 mg, 32.3% yield) as a white solid. ¹H NMR (400 MHz, DMSO-d6) δ 10.54 (s, 1H), 8.69-8.53 (m, 2H), 8.32 (s, 1H), 8.14 (s, 1H), 7.75 (d, J=8.7, 1H), 7.54 (d, J=8.7 Hz, 1H), 7.44 (s, 1H), 7.39-7.30 (m, 3H), 7.27 (s, 1H), 7.04-6.96 (m, 2H), 4.21-4.13 (m, 4H), 3.96 (s, 3H), 3.90 (t, J=6.6 Hz, 2H), 3.41 (s, 4H), 3.16 (t, J=12.6 Hz, 2H), 2.99-2.96 (m, 2H), 2.76-2.73 (m, 4H), 2.66-2.56 (m, 1H), 2.48-2.45 (m, 3H), 2.35-2.27 (m, 6H), 2.04-1.90 (m, 2H), 1.80-1.73 (m, 4H), 1.67-1.55 (m, 2H), 1.52-1.44 (m, 4H), 1.38 (t, J=6.9 Hz, 3H), 1.33-1.27 (m, 3H); MS: m/z [M+H]⁺ 1025, 96.7% purity. Prep HPLC purification conditions: Column: XBridge Prep OBD C18 Column, 30×150 mm, 5 μm; Mobile Phase A: Water (10 mmol/L NH₄HCO_{3+0.1}% NH₃·H₂O), Mobile Phase B: ACN; Flow rate: 60 mL/min; Gradient: 47% B to 57% B in 10 min, 57% B; RT=9.4 min.

### Example 43

### N-(1-(5-(3-cyano-6-ethoxypyrazolo[1,5-a]pyridin-4-yl)pyridin-2-yl)-4-((4-(4-(4-(3-(2,4-dioxotetrahydropyrimidin-1(2H)-yl)-1-methyl-1H-indazol-6-yl)piperazin-1-yl)butanoyl)piperazin-1-yl)methyl)piperidin-4-yl)-2,5-difluorobenzamide

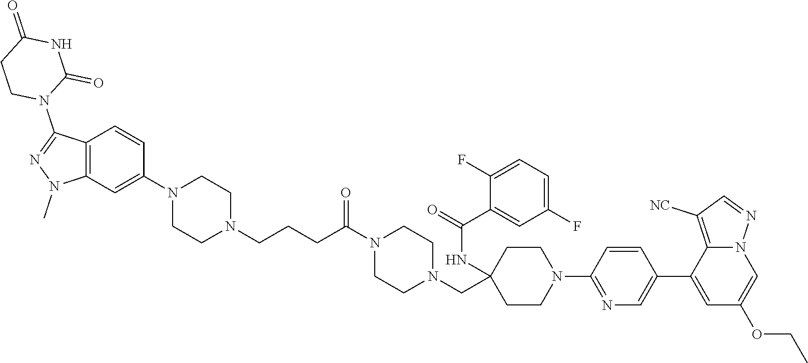 [SMILES: CCOc1cc(-c2ccc(N3CCC(CN4CCN(C(=O)CCCN5CCN(c6ccc7c(N8CCC(=O)NC8=O)nn(C)c7c6)CC5)CC4)(NC(=O)c4cc(F)ccc4F)CC3)nc2)c2c(C#N)cnn2c1]

The title compound was prepared in 21.4% overall yield as a white solid according to the preparation of Example 40 using 1-(1-methyl-6-(piperidin-4-yl)-1H-indazol-3-yl)dihydropyrimidine-2,4(1H,3H)-dione and tert-butyl 4-oxobutanoate in STEP A. 1H NMR (400 MHz, DMSO-d6) δ 8.65 (d, J=2.5 Hz, 1H), 8.57 (s, 1H), 8.32 (d, J=2.5 Hz, 1H), 8.15 (s, 1H), 7.75 (dd, J=8.9, 2.6 Hz, 1H), 7.54 (d, J=8.9 Hz, 1H), 7.43 (s, 1H), 7.37-7.31 (m, 3H), 7.27 (d, J=2.5 Hz, 1H), 7.06-6.93 (m, 2H), 4.21-4.13 (m, 4H), 3.96 (s, 3H), 3.90 (t, J=6.7 Hz, 2H), 3.45-3.41 (m, 4H), 3.16 (t, J=12.5 Hz, 2H), 2.99-2.95 (m, 2H), 2.80-2.69 (m, 4H), 2.65-2.59 (m, 1H), 2.56-2.52 (m, 1H), 2.49-2.47 (m, 2H), 2.35-2.29 (m, 6H), 2.05-1.92 (m, 2H), 1.84-1.56 (m, 8H), 1.38 (t, J=6.9 Hz, 3H); Analytical LCMS ESI-MS(+) m/z 996.6 [M+H]⁺, RT=1.65 min, purity: 95% (Method P). Prep HPLC purification conditions: Column: XBridge Prep OBD C18 Column, 30*150 mm, 5 μm; Mobile Phase A: Water (10 mmol/L NH₄HCO_{3+0.1}% NH₃·H₂O), Mobile Phase B: ACN; Flow rate: 60 mL/min; Gradient: 42% B to 52% B in 10 min, 52% B; RT=9.08 min.

### Example 44

### N-(1-(5-(3-cyano-6-ethoxypyrazolo[1,5-a]pyridin-4-yl)pyridin-2-yl)-4-((4-(2-(4-(3-(2,4-dioxotetrahydropyrimidin-1(2H)-yl)-1-methyl-1H-indazol-6-yl)piperidin-1-yl)acetyl)piperazin-1-yl)methyl)piperidin-4-yl)-2,5-difluorobenzamide

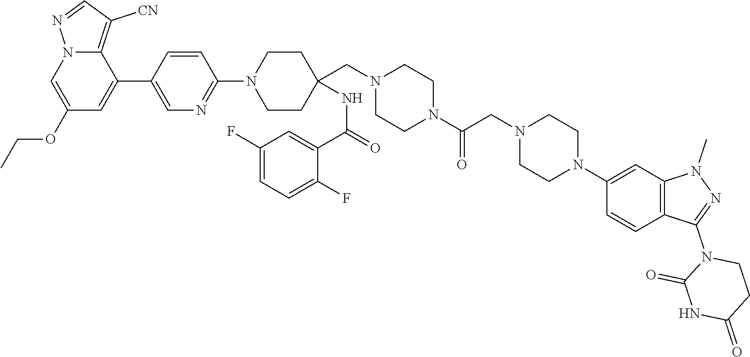 [SMILES: CCOc1cc(-c2ccc(N3CCC(CN4CCN(C(=O)CN5CCN(c6ccc7c(N8CCC(=O)NC8=O)nn(C)c7c6)CC5)CC4)(NC(=O)c4cc(F)ccc4F)CC3)nc2)c2c(C#N)cnn2c1]

### A. Tert-butyl 2-(4-(3-(2,4-dioxotetrahydropyrimidin-1(2H)-yl)-1-methyl-1H-indazol-6-yl)piperidin-1-yl)acetate

To a solution of 1-[1-methyl-6-(4-piperidyl) indazol-3-yl]hexahydropyrimidine-2,4-dione (60 mg, 0.18 mmol), tert-butyl 2-oxoacetate (47.7 mg, 0.37 mmol) in DCM (10 mL) were added DIEA (0.03 mL, 0.37 mmol), and AcOH (0.02 mL, 0.37 mmol). The mixture was stirred at rt for 1 h, NaBH(OAc)₃ (77.69 mg, 0.37 mmol) was added at 0 °C, the mixture was stirred at rt for another 1 h. After evaporation of the solvent, the crude was purified by reversed column chromatography to afford the desired product (60 mg, 74.2% yield) as a yellow oil. MS: m/z [M+H]⁺ 442.

### B. 2-(4-(3-(2,4-dioxotetrahydropyrimidin-1(2H)-yl)-1-methyl-1H-indazol-6-yl)piperidin-1-yl)acetic acid

To a solution of tert-butyl 2-[4-[3-(2,4-dioxohexahydropyrimidin-1-yl)-1-methyl-indazol-6-yl]-1-piperidyl]acetate (60 mg, 0.14 mmol) in DCM (0.50 mL) was added TFA (0.5 mL). The mixture was stirred at rt for 1 h. The reaction was concentrated to afford crude product which was used in the next step without further purification. MS: m/z [M+H]⁺ 386.

### C. N-(1-(5-(3-cyano-6-ethoxypyrazolo[1,5-a]pyridin-4-yl)pyridin-2-yl)-4-((4-(2-(4-(3-(2,4-dioxotetrahydropyrimidin-1(2H)-yl)-1-methyl-1H-indazol-6-yl)piperidin-1-yl)acetyl)piperazin-1-yl)methyl)piperidin-4-yl)-2,5-difluorobenzamide

To a solution of N-[1-[5-(3-cyano-6-ethoxy-pyrazolo[1,5-a]pyridin-4-yl)-2-pyridyl]-4-(piperazin-1-ylmethyl)-4-piperidyl]-2,5-difluoro-benzamide (35 mg, 0.06 mmol), 2-[4-[3-(2,4-dioxohexahydropyrimidin-1-yl)-1-methyl-indazol-6-yl]-1-piperidyl]acetic acid (22.46 mg, 0.06 mmol) in DMA (5 mL) were added HATU (33.23 mg, 0.09 mmol), and DIEA (0.01 mL, 0.17 mmol). The mixture was stirred at rt for 2 h. The crude was purified by prep-HPLC to afford the titled compound (17.4 mg, 30% yield) as an off-white solid. ¹H NMR (400 MHz, DMSO-d6) δ 8.66 (d, J=2.5 Hz, 1H), 8.58 (s, 1H), 8.33 (d, J=2.5 Hz, 1H), 8.17 (s, 1H), 7.76 (dd, J=8.9, 2.6 Hz, 1H), 7.55 (d, J=8.9 Hz, 1H), 7.43 (s, 1H), 7.38-7.32 (m, 3H), 7.28 (d, J=2.5 Hz, 1H), 7.05-6.95 (m, 2H), 4.26-4.11 (m, 4H), 3.96 (s, 3H), 3.90 (t, J=6.7 Hz, 2H), 3.56-3.54 (m, 2H), 3.43-3.40 (m, 2H), 3.19-3.13 (m, 4H), 2.95-2.92 (m, 2H), 2.80-2.71 (m, 4H), 2.66-2.56 (m, 3H), 2.49-2.46 (m, 2H), 2.36-2.33 (m, 2H), 2.19-2.06 (m, 2H), 1.86-1.57 (m, 6H), 1.38 (t, J=6.9 Hz, 3H); Analytical LCMS ESI-MS(+) m/z 968.6 [M+H]⁺, RT=1.36 min, purity: 96.8% (Method P); Prep HPLC purification conditions: Column: Xselect CSH C18 OBD Column 30×150 mm 5 μm; Mobile Phase A: Water (10 mmol/L NH₄HCO_{3+0.1}% NH₃·H₂O), Mobile Phase B: ACN; Flow rate: 60 mL/min; Gradient: 33% B to 63% B in 10 min, 63% B; RT1=9.85 min.

### Example 45

### N-(1-(5-(3-cyano-6-ethoxypyrazolo[1,5-a]pyridin-4-yl)pyridin-2-yl)-4-((4-(3-(4-(3-(2,4-dioxotetrahydropyrimidin-1(2H)-yl)-1-methyl-1H-indazol-6-yl)piperidin-1-yl)propanoyl)piperazin-1-yl)methyl)piperidin-4-yl)-2,5-difluorobenzamide

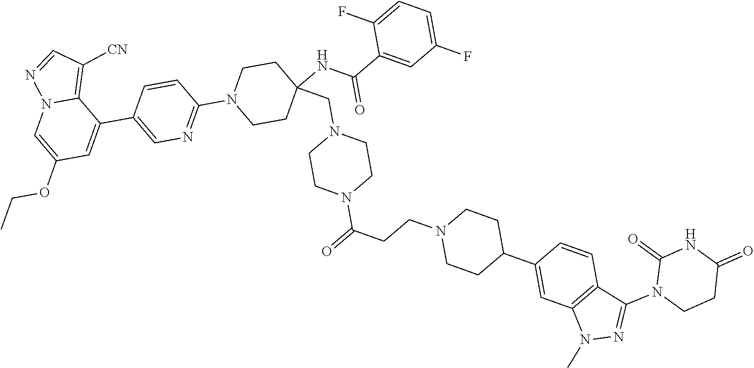 [SMILES: CCOc1cc(-c2ccc(N3CCC(CN4CCN(C(=O)CCN5CCC(c6ccc7c(N8CCC(=O)NC8=O)nn(C)c7c6)CC5)CC4)(NC(=O)c4cc(F)ccc4F)CC3)nc2)c2c(C#N)cnn2c1]

The title compound was prepared in 21.4% overall yield as a white solid according to the preparation of Example 39 using 1-(1-methyl-6-(piperidin-4-yl)-1H-indazol-3-yl)dihydropyrimidine-2,4(1H,3H)-dione in STEP A. ¹H NMR (400 MHz, DMSO-d6) δ 10.56 (s, 1H), 8.66 (d, J=2.5 Hz, 1H), 8.58 (s, 1H), 8.33 (d, J=2.5 Hz, 1H), 8.17 (s, 1H), 7.76 (dd, J=8.8, 2.6 Hz, 1H), 7.54 (d, J=8.8 Hz, 1H), 7.44 (s, 1H), 7.38-7.32 (m, 3H), 7.27 (d, J=2.5 Hz, 1H), 7.07-6.92 (m, 2H), 4.25-4.11 (m, 4H), 3.96 (s, 3H), 3.90 (t, J=6.7 Hz, 2H), 3.43-3.41 (m, 4H), 3.20-3.11 (m, 2H), 3.01-2.97 (m, 2H), 2.78-2.70 (m, 4H), 2.62-2.55 (m, 5H), 2.49-2.45 (m, 4H), 2.36-2.32 (m, 2H), 2.09-2.02 (m, 2H), 1.84-1.67 (m, 4H), 1.64-1.57 (m, 2H), 1.38 (t, J=6.9 Hz, 3H); Analytical LCMS ESI-MS(+) m/z 492.0 [M+2H]²⁺, RT=1.90 min, purity: 98.0% (Method P); Prep HPLC purification conditions: Column: Xselect CSH C18 OBD Column 30×150 mm 5 μm; Mobile Phase A: Water (10 mmol/L NH₄HCO_{3+0.1}% NH₃·H₂O), Mobile Phase B: ACN; Flow rate: 60 mL/min; Gradient: 27% B to 57% B in 10 min, 57% B; RT=10.82 min.

### Example 46

### N-(1-(5-(3-cyano-6-ethoxypyrazolo[1,5-a]pyridin-4-yl)pyridin-2-yl)-4-((4-(4-(4-(3-(2,4-dioxotetrahydropyrimidin-1(2H)-yl)-1-methyl-1H-indazol-6-yl)piperidin-1-yl)butanoyl)piperazin-1-yl)methyl)piperidin-4-yl)-2,5-difluorobenzamide

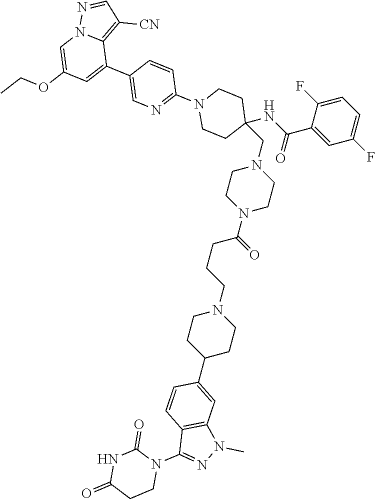 [SMILES: CCOc1cc(-c2ccc(N3CCC(CN4CCN(C(=O)CCCN5CCC(c6ccc7c(N8CCC(=O)NC8=O)nn(C)c7c6)CC5)CC4)(NC(=O)c4cc(F)ccc4F)CC3)nc2)c2c(C#N)cnn2c1]

The title compound was prepared in 40.7% overall yield as a white solid according to the preparation of Example 39 using 1-(1-methyl-6-(piperidin-4-yl)-1H-indazol-3-yl)dihydropyrimidine-2,4(1H,3H)-dione in STEP A. ¹H NMR (400 MHz, DMSO-d6) δ 8.67 (d, J=2.0 Hz, 1H), 8.59 (s, 1H), 8.36 (d, J=2.5 Hz, 1H), 7.82 (dd, J=8.9, 2.5 Hz, 1H), 7.65-7.60 (m, 1H), 7.53-7.46 (m, 1H), 7.44-7.37 (m, 3H), 7.28 (d, J=2.3 Hz, 1H), 7.10-7.01 (m, 2H), 4.33-4.23 (m, 2H), 4.16 (q, J=6.9 Hz, 2H), 3.99 (s, 3H), 3.92 (t, J=6.7 Hz, 2H), 3.68-3.60 (m, 3H), 3.54-3.49 (m, 9H), 3.27-3.17 (m, 3H), 3.16-3.06 (s, 5H), 3.05-2.96 (m, 1H), 2.76 (t, J=6.6 Hz, 2H), 2.46-2.39 (m, 2H), 2.16-2.06 (m, 2H), 2.04-1.89 (m, 4H), 1.79-1.67 (m, 2H), 1.39 (t, J=6.9 Hz, 3H). MS: m/z [M+H]⁺ 996.6, purity: 99.1%; Prep-HPLC purification condition: Column: SunFire C18 OBD Prep Column, 19×250 mm, 5 μm; Mobile Phase A: Water (0.05% TFA), Mobile Phase B: ACN; Flow rate: 25 mL/min; Gradient: 15% B to 45% B in 6.5 min, 45% B; RT=6.1 min.

### Example 47

### N-[1-[5-(3-cyano-6-ethoxy-pyrazolo[1,5-a]pyridin-4-yl)-2-pyridyl]-4-[[1-[6-[4-[2-(2,6-dioxo-3-piperidyl)-1-oxo-isoindolin-5-yl]piperazin-1-yl]hexyl]triazol-4-yl]methoxymethyl]-4-piperidyl]-2,5-difluoro-benzamide

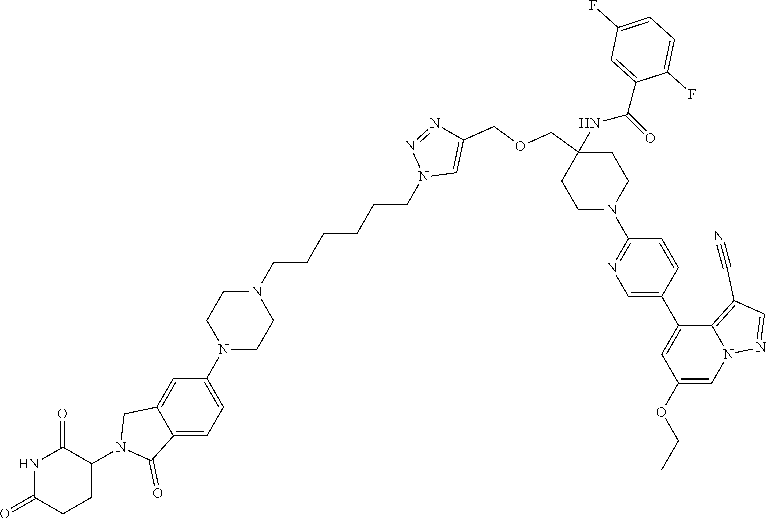 [SMILES: CCOc1cc(-c2ccc(N3CCC(COCc4cn(CCCCCCN5CCN(c6ccc7c(c6)CN(C6CCC(=O)NC6=O)C7=O)CC5)nn4)(NC(=O)c4cc(F)ccc4F)CC3)nc2)c2c(C#N)cnn2c1]

### A. 3-[5-[4-(6-azidohexyl)piperazin-1-yl]-1-oxo-isoindolin-2-yl]piperidine-2,6-dione

To the mixture of 3-(1-oxo-5-piperazin-1-yl-isoindolin-2-yl)piperidine-2,6-dione (90 mg, 0.27 mmol) in MeCN (20 mL) were added 1-azido-6-bromo-hexane (170 mg, 0.82 mmol), NaI (4.11 mg, 0.03 mmol), DIPEA (0.14 mL, 0.82 mmol), the mixture was warmed up to 80 °C for overnight. The reaction was concentrated and purified by Prep-TLC to afford the desired product (90 mg, 72.4% yield) as a yellow solid. MS: m/z [M+H]⁺ 454.

### B. N-[1-[5-(3-cyano-6-ethoxy-pyrazolo[1,5-a]pyridin-4-yl)-2-pyridyl]-4-[[1-[6-[4-[2-(2,6-dioxo-3-piperidyl)-1-oxo-isoindolin-5-yl]piperazin-1-yl]hexyl]triazol-4-yl]methoxymethyl]-4-piperidyl]-2,5-difluoro-benzamide

Under a N₂ atmosphere, to a stirred solution of 3-[5-[4-(6-azidohexyl)piperazin-1-yl]-1-oxo-isoindolin-2-yl]piperidine-2,6-dione (72 mg, 0.16 mmol) in THF (4 mL) and H₂O (2 mL) was added N-[1-[5-(3-cyano-6-ethoxy-pyrazolo[1,5-a]pyridin-4-yl)-2-pyridyl]-4-(prop-2-ynoxymethyl)-4-piperidyl]-2,5-difluoro-benzamide (90 mg, 0.16 mmol) at 0 °C, a solution of CuSO₄ (13 mg, 0.08 mmol) in H₂O (1 mL) was subsequently added followed by addition of a solution of sodium ascorbate (16 mg, 0.08 mmol) in H₂O (1 mL), the resulting mixture was stirred at rt for overnight. The reaction was concentrated and purified by reversed phase column (0.5% TFA in water/MeCN) followed by Prep-HPLC purification to afford the titled compound (23.3 mg, 14.3% yield) as a white solid. ¹H NMR (300 MHz, DMSO-d6) δ 8.64 (d, J=2.1 Hz, 1H), 8.57 (s, 1H), 8.32 (d, J=2.6 Hz, 1H), 8.11-8.09 (m, 2H), 7.75 (dd, J=8.8, 2.6 Hz, 1H), 7.52-7.49 (m, 1H), 7.42-7.29 (m, 3H), 7.27 (d, J=2.1 Hz, 1H), 7.04-7.02 (m, 2H), 6.97 (d, J=8.8 Hz, 1H), 5.04 (dd, J=13.2, 5.1 Hz, 1H), 4.56 (s, 2H), 4.38-4.25 (m, 3H), 4.21-4.12 (m, 5H), 3.70 (s, 2H), 3.24-3.13 (m, 6H), 2.97-2.84 (m, 1H), 2.64-2.57 (m, 1H), 2.57-2.56 (m, H), 2.47-2.43 (m, 3H), 2.35-2.14 (m, 5H), 2.00-1.91 (m, 1H), 1.85-1.75 (m, 2H), 1.68-1.54 (m, 2H), 1.47-1.35 (m, 5H), 1.29-1.23 (m, 4H); MS: m/z 1024.3, purity: 99.1%. Prep-HPLC purification conditions: Column: XSelect CSH Fluoro Phenyl, 30×150 mm, 5 μm; Mobile Phase A: Water (0.05% TFA), Mobile Phase B: ACN; Flow rate: 60 mL/min; Gradient: 2% B to 29% B in 10 min, 29% B; RT=8.83 min.

### Example 48

### N-[1-[5-(3-cyano-6-ethoxy-pyrazolo[1,5-a]pyridin-4-yl)-2-pyridyl]-4-[[1-[4-[4-[2-(2,6-dioxo-3-piperidyl)-1-oxo-isoindolin-5-yl]piperazin-1-yl]butyl]triazol-4-yl]methoxymethyl]-4-piperidyl]-2,5-difluoro-benzamide

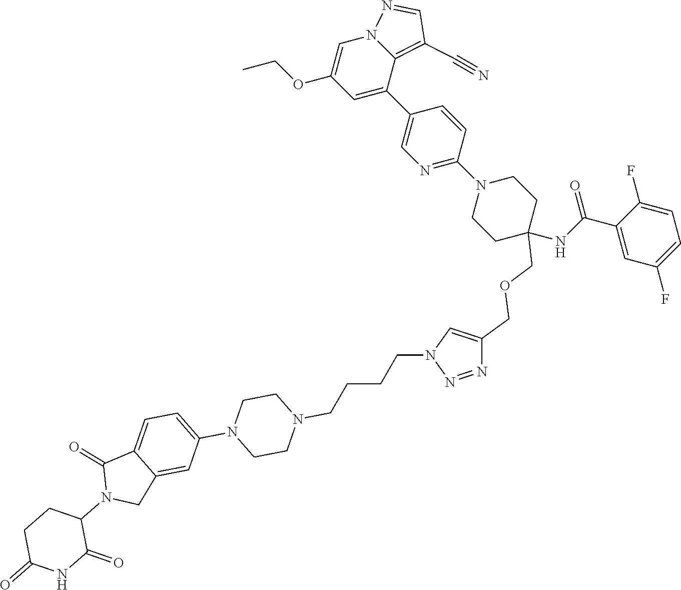 [SMILES: CCOc1cc(-c2ccc(N3CCC(COCc4cn(CCCCN5CCN(c6ccc7c(c6)CN(C6CCC(=O)NC6=O)C7=O)CC5)nn4)(NC(=O)c4cc(F)ccc4F)CC3)nc2)c2c(C#N)cnn2c1]

The title compound was prepared in 36.8% overall yield as a white solid according to the preparation of Example 47 using 1-azido-4-bromo-butane in STEP A. ¹H NMR (300 MHz, DMSO-d6) δ 10.88 (brs, 1H), 8.65 (d, J=2.1 Hz, 1H), 8.57 (s, 1H), 8.32 (d, J=2.6 Hz, 1H), 8.10-8.11 (m, 2H), 7.75 (dd, J=8.9, 2.6 Hz, 1H), 7.51-7.48 (m, 1H), 7.38-7.30 (m, 3H), 7.27 (d, J=2.1 Hz, 1H), 7.04-7.01 (m, 2H), 6.96 (d, J=8.9 Hz, 1H), 5.07-5.01 (m, 1H), 4.56 (s, 2H), 4.42-4.25 (m, 3H), 4.24-4.09 (m, 5H), 3.70 (s, 2H), 3.24-3.12 (m, 6H), 2.96-2.84 (m, 1H), 2.63-2.54 (m, 1H), 2.49-2.35 (m, 4H), 2.35-2.24 (m, 5H), 2.00-1.92 (m, 1H), 1.97-1.78 (m, 2H), 1.65-1.56 (m, 2H), 1.46-1.36 (m, 5H); Analytical LCMS ESI-MS(+) m/z 1006.2 [M+H]⁺, RT=1.13 min, purity: 98.9% (Method Q); Prep HPLC purification conditions: Column: XBridge Prep OBD C18 Column, 30×150 mm, 5 μm; Mobile Phase A: Water (10 mmol/L NH₄HCO_{3+0.1}% NH₃·H₂O), Mobile Phase B: ACN; Flow rate: 60 mL/min; Gradient: 40% B to 50% B in 9 min, 50% B; RT=8.42 min.

### Example 49. B

### N-[1-[5-(3-cyano-6-ethoxy-pyrazolo[1,5-a]pyridin-4-yl)-2-pyridyl]-4-[[1-[2-[4-[2-(2,6-dioxo-3-piperidyl)-1-oxo-isoindolin-5-yl]piperazin-1-yl]ethyl]triazol-4-yl]methoxymethyl]-4-piperidyl]-2,5-difluoro-benzamide

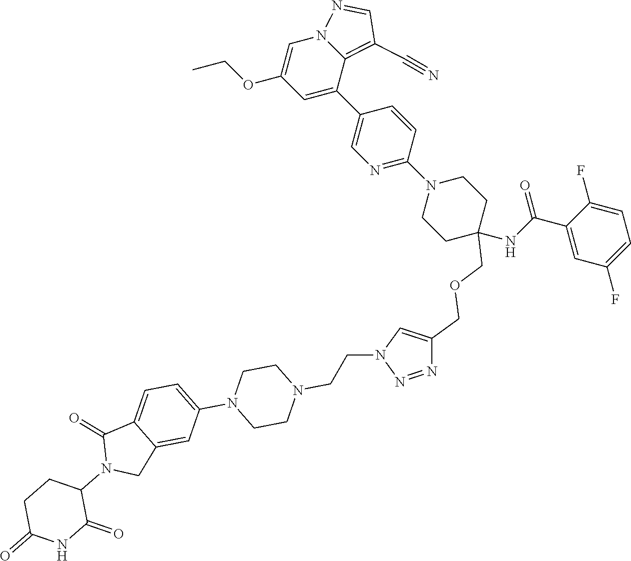 [SMILES: CCOc1cc(-c2ccc(N3CCC(COCc4cn(CCN5CCN(c6ccc7c(c6)CN(C6CCC(=O)NC6=O)C7=O)CC5)nn4)(NC(=O)c4cc(F)ccc4F)CC3)nc2)c2c(C#N)cnn2c1]

The title compound was prepared in 5.5% overall yield as a white solid according to the preparation of Example 47 using 1-azido-2-bromo-ethane in STEP A. ¹H NMR (400 MHz, DMSO-d6) δ 10.98 (s, 1H), 8.69 (s, 1H), 8.60 (s, 1H), 8.34 (s, 1H), 8.22-8.18 (m, 2H), 7.97 (s, 1H), 7.84 (d, J=9.0 Hz, 1H), 7.59 (d, J=8.4 Hz, 1H), 7.42-7.30 (m, 4H), 7.18-7.11 (m, 2H), 7.07 (d, J=9.0 Hz, 1H), 5.11-5.04 (m, 1H), 4.90-4.84 (m, 2H), 4.63 (s, 2H), 4.38-4.31 (m, 1H), 4.27-4.14 (m, 6H), 3.81-3.75 (m, 7H), 3.26-3.19 (m, 2H), 2.93-2.89 (m, 3H), 2.76-2.73 (m, 2H), 2.65-2.58 (m, 1H), 2.45-2.40 (m, 1H), 2.35-2.27 (m, 2H), 2.01-1.93 (m, 1H), 1.73-1.61 (m, 2H), 1.40 (t, J=7.1 Hz, 3H); Analytical LCMS ESI-MS(+) m/z 968.5 [M+H]⁺, RT=1.27 min, purity: 97.4% (Method Q). Prep HPLC purification conditions: Column: SunFire Prep C18 OBD Column, 19×150 mm, 5 μm; Mobile Phase A: Water (0.05% TFA), Mobile Phase B: ACN; Flow rate: 20 mL/min; Gradient: 25% B to 55% B in 6.3 min, 55% B; RT=5.53 min.

### Example 50

### N-(1-(5-(3-cyano-6-ethoxypyrazolo[1,5-a]pyridin-4-yl)pyridin-2-yl)-4-(((1-(3-(4-(2-(2,6-dioxopiperidin-3-yl)-1-oxoisoindolin-5-yl)piperazin-1-yl) propyl)-1H-1,2,3-triazol-4-yl) methoxy)methyl)piperidin-4-yl)-2,5-difluorobenzamide

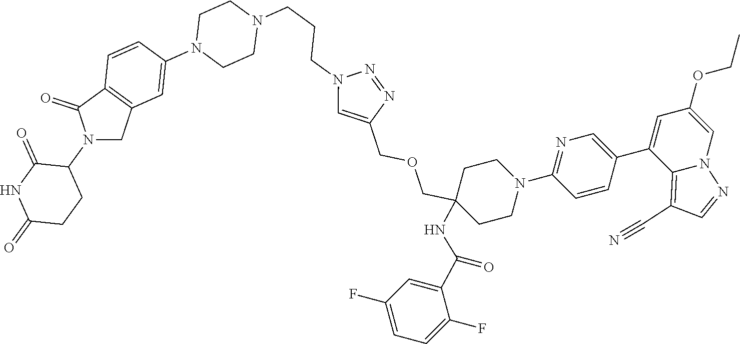 [SMILES: CCOc1cc(-c2ccc(N3CCC(COCc4cn(CCCN5CCN(c6ccc7c(c6)CN(C6CCC(=O)NC6=O)C7=O)CC5)nn4)(NC(=O)c4cc(F)ccc4F)CC3)nc2)c2c(C#N)cnn2c1]

### A. N-(1-(5-(3-cyano-6-ethoxypyrazolo[1,5-a]pyridin-4-yl)pyridin-2-yl)-4-((prop-2-yn-1-yloxy)methyl)piperidin-4-yl)-2,5-difluorobenzamide

To a stirred solution of N-[1-[5-(3-cyano-6-ethoxy-pyrazolo[1,5-a]pyridin-4-yl)-2-pyridyl]-4-(hydroxymethyl)-4-piperidyl]-2,5-difluoro-benzamide (500 mg, 0.94 mmol) in THF (30 mL) was added NaH (90.13 mg, 3.76 mmol) at 0 °C, and the resulting mixture was stirred at rt for 0.5 h, then 3-bromoprop-1-yne (335.07 mg, 2.82 mmol) was added, the mixture solution was stirred at rt for 48 h. The reaction was quenched by ice water, extracted with EtOAc, washed with brine, dried over sodium sulfate, filtered and concentrated. The residue was purified by column chromatography to afford the desired product (400 mg, 74.7% yield) as an off-white solid. MS: m/z [M+H]⁺ 571.

### B. 3-(5-(4-(3-azidopropyl)piperazin-1-yl)-1-oxoisoindolin-2-yl)piperidine-2,6-dione

To the mixture solution of 3-(1-oxo-5-piperazin-1-yl-isoindolin-2-yl)piperidine-2,6-dione (80 mg, 0.24 mmol) in ACN (8 mL) was added 1-azido-3-bromo-propane (120 mg, 0.73 mmol) and DIEA (0.07 mL, 0.9700 mmol), the mixture solution was stirred at 80 °C overnight. Then 1-azido-3-bromo-propane (80 mg, 0.49 mmol), DIEA (0.04 mL, 0.49 mmol) was added at rt and stirred at 80 °C overnight. The reaction was concentrated and purified by silica gel column to afford 3-[5-[4-(3-azidopropyl)piperazin-1-yl]-1-oxo-isoindolin-2-yl]piperidine-2,6-dione (60 mg, 0.14 mmol, 59.8% yield) as yellow solid. MS: m/z [M−H]⁺ 410.

### C. N-(1-(5-(3-cyano-6-ethoxypyrazolo[1,5-a]pyridin-4-yl)pyridin-2-yl)-4-(((1-(3-(4-(2-(2,6-dioxopiperidin-3-yl)-1-oxoisoindolin-5-yl)piperazin-1-yl) propyl)-1H-1,2,3-triazol-4-yl) methoxy)methyl)piperidin-4-yl)-2,5-difluorobenzamide

The mixture solution of 3-[5-[4-(3-azidopropyl)piperazin-1-yl]-1-oxo-isoindolin-2-yl]piperidine-2,6-dione (60 mg, 0.15 mmol) and N-[1-[5-(3-cyano-6-ethoxy-pyrazolo[1,5-a]pyridin-4-yl)-2-pyridyl]-4-(prop-2-ynoxymethyl)-4-piperidyl]-2,5-difluoro-benzamide (83 mg, 0.15 mmol) in THF (4 mL) and water (4 mL) was degassed under N₂ for 5 times, and then cooled down to 0 °C, CuSO₄ (7.0 mg, 0.04 mmol), and Sodium ascorbate (10 mg, 0.05 mmol) was added successively. The resulting mixture was stirred at rt under N₂ atmosphere for 4 h. Upon completion, the reaction was concentrated and purified by Prep-HPLC to afford the titled compound (17.3 mg, 11.7% yield) as an off-white solid. ¹H NMR (400 MHz, DMSO-d6) δ 8.66 (s, 1H), 8.58 (brs, 1H), 8.32 (s, 1H), 8.20 (s, 1H), 8.14 (brs, 1H), 7.91-7.89 (m, 1H), 7.59-7.57 (m, 1H), 7.37-7.31 (m, 4H), 7.17-7.11 (m, 3H), 5.06-5.02 (m, 1H), 4.59 (s, 2H), 4.50-4.46 (m, 2H), 4.39-3.93 (m, 8H), 3.78-3.68 (m, 2H), 3.65-3.63 (m, 2H), 3.31-3.02 (m, 8H), 2.94-2.82 (m, 1H), 2.64-2.56 (m, 1H), 2.31-3.02 (m, 5H), 2.01-1.93 (m, 1H), 1.69-1.62 (m, 2H), 1.38 (t, J=7.0 Hz, 3H); Analytical LCMS ESI-MS(+) m/z 982.5 [M+H]⁺, RT=1.27 min, purity: 100% (Method Q). Prep HPLC purification conditions: Column: SunFire Prep C18 OBD Column, 19×150 mm, 5 μm; Mobile Phase A: water (0.05% TFA), Mobile Phase B: ACN; Flow rate: 20 mL/min; Gradient: 25% B to 50% B in 5.5 min, 50% B; RT=5.53 min.

### Example 51

### N-[4-benzyl-1-[5-(3-cyano-6-ethoxy-pyrazolo[1,5-a]pyridin-4-yl)-2-pyridyl]-4-piperidyl]-4-[3-[4-[2-(2,6-dioxo-3-piperidyl)-3-oxo-isoindolin-5-yl]-1-piperidyl]propanoyl]piperazine-1-carboxamide

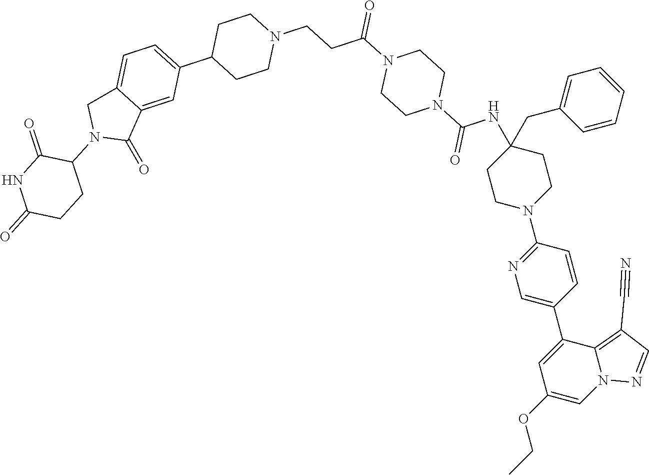 [SMILES: CCOc1cc(-c2ccc(N3CCC(Cc4ccccc4)(NC(=O)N4CCN(C(=O)CCN5CCC(c6ccc7c(c6)C(=O)N(C6CCC(=O)NC6=O)C7)CC5)CC4)CC3)nc2)c2c(C#N)cnn2c1]

### A. Tert-butyl 4-(tert-butylsulfinylamino)-4-cyano-piperidine-1-carboxylate

To a solution of tert-butyl 4-oxopiperidine-1-carboxylate (5 g, 25.09 mmol) in THF (120 mL) were added Ti(OEt)₄ (10.52 mL, 50.19 mmol), t-BuSONH₂ (3218.57 mg, 26.6 mmol). The mixture was stirred at 70 °C for 20 h under N₂. The red solution was cooled, TMSCN (3.45 mL, 27.6 mmol) was added, and the solution was refluxed for 3 h. It was concentrated and purified by column chromatography (PE:EA=1:1 to EA) to afford the desired product (5 g, 60.5% yield) as a white solid. MS: m/z [M+H]⁺ 330.

### B. Tert-butyl 4-benzyl-4-(tert-butylsulfinylamino)piperidine-1-carboxylate

To a solution of tert-butyl 4-(tert-butylsulfinylamino)-4-cyano-piperidine-1-carboxylate (3.5 g, 10.62 mmol) in THF (40 mL) was added MgBnCl (2M in THF) (13 mL, 23.9 mmol). The reaction was placed under vacuum, sonicated and backfilled with nitrogen. The mixture was stirred at 80 °C for 5 h. It was concentrated and purified by column chromatography (PE:EA=1:2 to EA) to afford the desired (2 g, 47.7% yield) as a yellow solid. MS: m/z [M+H]⁺ 395.

### C. 4-benzylpiperidin-4-amine

To the mixture solution of tert-butyl 4-benzyl-4-(tert-butylsulfinylamino)piperidine-1-carboxylate (2000 mg, 5.07 mmol) in Ethyl acetate (20 mL) was added HCl (2.0 M in EA) (539 mg, 5.07 mmol) at rt, the mixture solution was stirred at rt for 3 h. The reaction was filtered, and the filter cake was dried to afford 4-benzylpiperidin-4-amine (960 mg, 97.9% yield) as an off white solid. MS: m/z [M+H]⁺ 191.

### D. 4-[6-(4-amino-4-benzyl-1-piperidyl)-3-pyridyl]-6-ethoxy-pyrazolo[1,5-a]pyridine-3-carbonitrile

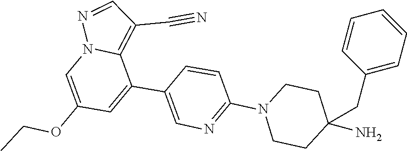 [SMILES: CCOc1cc(-c2ccc(N3CCC(N)(Cc4ccccc4)CC3)nc2)c2c(C#N)cnn2c1]

To the mixture solution of 4-benzylpiperidin-4-amine (26.29 mg, 0.14 mmol) and 6-ethoxy-4-(6-fluoro-3-pyridyl) pyrazolo[1,5-a]pyridine-3-carbonitrile (30 mg, 0.11 mmol) in DMA (2 mL) was added K₂CO₃ (44 mg, 0.32 mmol), the mixture was stirred at 100 °C for overnight. The reaction was purified by Prep-HPLC to afford the desired product (13.6 mg, 27.6% yield) as an off-white solid. ¹H NMR (300 MHz, DMSO-d6) δ 8.67 (d, J=2.1 Hz, 1H), 8.59 (s, 1H), 8.35 (d, J=2.5 Hz, 1H), 7.93 (brs, 2H), 7.81 (dd, J=8.8, 2.5 Hz, 1H), 7.43-7.27 (m, 6H), 7.02 (d, J=8.8 Hz, 1H), 4.16-4.12 (m, 2H), 3.94-3.73 (m, 4H), 3.08 (s, 2H), 1.84-1.71 (m, 4H), 1.38 (t, J=6.9 Hz, 3H); MS: m/z [M+H]⁺ 453. Prep HPLC purification conditions: Column: Xselect CSH C18 OBD Column 30×150 mm 5 μm; Mobile Phase A: Water (0.05% TFA), Mobile Phase B: ACN; Flow rate: 60 mL/min; Gradient: 8% B to 38% B in 10 min, 38% B; RT=9.35 min.

### E. 4-[6-(4-benzyl-4-isocyanato-1-piperidyl)-3-pyridyl]-6-ethoxy-pyrazolo[1,5-a]pyridine-3-carbonitrile

To a stirred solution of 4-[6-(4-amino-4-benzyl-1-piperidyl)-3-pyridyl]-6-ethoxy-pyrazolo[1,5-a]pyridine-3-carbonitrile (120.0 mg, 0.27 mmol) in DCM (10 mL) were added BST (31.5 mg, 0.11 mmol) and DIPEA (0.09 mL, 0.53 mmol) at 0 °C, the mixture solution was stirred at rt for 2 h. The reaction was used for next step directly without further work-up. MS: m/z [M+H]⁺ 479.

### F. Tert-butyl 4-[[4-benzyl-1-[5-(3-cyano-6-ethoxy-pyrazolo[1,5-a]pyridin-4-yl)-2-pyridyl]-4-piperidyl]carbamoyl]piperazine-1-carboxylate

To the mixture solution of 4-[6-(4-benzyl-4-isocyanato-1-piperidyl)-3-pyridyl]-6-ethoxy-pyrazolo[1,5-a]pyridine-3-carbonitrile (150 mg, 0.31 mmol) in DCM (5 mL) was added tert-butyl piperazine-1-carboxylate (175.14 mg, 0.94 mmol) at 0 °C, and the mixture solution was stirred at rt overnight. It was concentrated and purified by silica gel column to afford the desired product (150 mg, 72% yield) as a white solid. MS: m/z: [M+H]⁺ 665.

### G. N-[4-benzyl-1-[5-(3-cyano-6-ethoxy-pyrazolo[1,5-a]pyridin-4-yl)-2-pyridyl]-4-piperidyl]piperazine-1-carboxamide

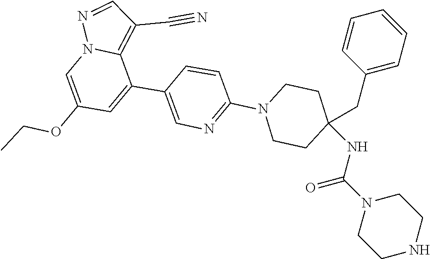 [SMILES: CCOc1cc(-c2ccc(N3CCC(Cc4ccccc4)(NC(=O)N4CCNCC4)CC3)nc2)c2c(C#N)cnn2c1]

To the mixture solution of tert-butyl 4-[[4-benzyl-1-[5-(3-cyano-6-ethoxy-pyrazolo[1,5-a] pyridin-4-yl)-2-pyridyl]-4-piperidyl]carbamoyl]piperazine-1-carboxylate (140 mg, 0.2100 mmol) in ethyl acetate (4 mL) was added HCl (g) (4 mL, 0.2100 mmol) in EtOAc, and the mixture solution was stirred at rt for 2 h. It was concentrated and purified by Prep-HPLC to afford the desired product (22 mg, yield 18.4%) as a white solid. ¹H NMR (300 MHz, DMSO-d6) δ 8.73 (brs, 2H), 8.67 (d, J=2.1 Hz, 1H), 8.58 (s, 1H), 8.31 (d, J=2.5 Hz, 1H), 7.79 (dd, J=8.9, 2.5 Hz, 1H), 7.36-7.24 (m, 3H), 7.28-7.17 (m, 1H), 7.15-7.05 (m, 2H), 7.01 (d, J=9.0 Hz, 1H), 5.84 (s, 1H), 4.20-4.10 (m, 4H), 3.54-3.51 (m, 4H), 3.14-3.05 (m, 8H), 2.20-2.17 (m, 2H), 1.60-1.51 (s, 2H), 1.38 (t, J=6.9 Hz, 3H); MS: m/z [M+H]⁺ 565.3. Prep HPLC purification conditions: Column: XSelect CSH Fluoro Phenyl, 30×150 mm, 5 μm; Mobile Phase A: Water (0.05% TFA), Mobile Phase B: ACN; Flow rate: 60 mL/min; Gradient: 2% B to 29% B in 10 min, 29% B; RT=8.83 min.

### H. N-[4-benzyl-1-[5-(3-cyano-6-ethoxy-pyrazolo[1,5-a]pyridin-4-yl)-2-pyridyl]-4-piperidyl]-4-[3-[4-[2-(2,6-dioxo-3-piperidyl)-3-oxo-isoindolin-5-yl]-1-piperidyl]propanoyl]piperazine-1-carboxamide

To the mixture solution of 3-[4-[2-(2,6-dioxo-3-piperidyl)-3-oxo-isoindolin-5-yl]-1-piperidyl]propanoic acid (40 mg, 0.10 mmol), N-[4-benzyl-1-[5-(3-cyano-6-ethoxy-pyrazolo[1,5-a]pyridin-4-yl)-2-pyridyl]-4-piperidyl]piperazine-1-carboxamide (56.55 mg, 0.10 mmol) and HATU (57.11 mg, 0.15 mmol) was added DIEA (0.02 mL, 0.30 mmol) at 0 °C, and the mixture was stirred at 0 °C for 2 h. The reaction was purified by Prep-HPLC to afford the titled compound (40.7 mg, 42.9% yield) as a white solid. ¹H NMR (300 MHz, DMSO-d6) δ 10.97 (s, 1H), 8.64 (d, J=2.1 Hz, 1H), 8.57 (s, 1H), 8.31 (d, J=2.6 Hz, 1H), 7.73 (dd, J=9.0, 2.6 Hz, 1H), 7.58-7.49 (m, 3H), 7.34-7.05 (m, 6H), 6.94 (d, J=9.0 Hz, 1H), 5.65 (s, 1H), 5.11 (dd, J=13.2, 5.1 Hz, 1H), 4.45-4.34 (m, 1H), 4.33-4.23 (m, 1H), 4.21-4.04 (m, 4H), 3.05-3.36 (m, 6H), 3.30-3.25 (m, 2H), 3.15-2.82 (m, 7H), 2.66-2.51 (m, 6H), 2.48-2.28 (m, 1H), 2.24-1.94 (m, 5H), 1.83-1.45 (m, 6H), 1.38 (t, J=6.9 Hz, 3H); MS: m/z: [M+H]⁺ 946.3, purity: 99.7%. Prep HPLC purification conditions: Column: XBridge Prep OBD C18 Column, 30×150 mm, 5 μm; Mobile Phase A: Water (10 mmol/L NH₄HCO_{3+0.1}% NH₃·H₂O), Mobile Phase B: ACN; Flow rate: 60 mL/min; Gradient: 45% B to 46% B in 15 min, 46% B; RT=12.63 min.

### Example 52

### N-[4-benzyl-1-[5-(3-cyano-6-ethoxy-pyrazolo[1,5-a]pyridin-4-yl)-2-pyridyl]-4-piperidyl]-1-[3-[4-[2-(2,6-dioxo-3-piperidyl)-3-oxo-isoindolin-5-yl]-1-piperidyl]propanoyl]piperidine-4-carboxamide

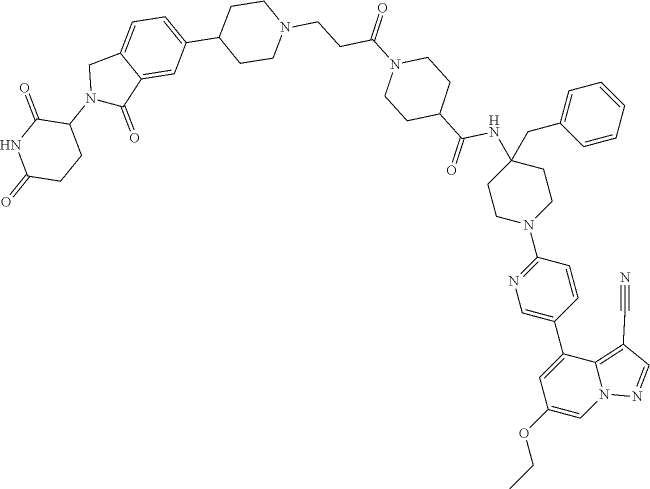 [SMILES: CCOc1cc(-c2ccc(N3CCC(Cc4ccccc4)(NC(=O)C4CCN(C(=O)CCN5CCC(c6ccc7c(c6)C(=O)N(C6CCC(=O)NC6=O)C7)CC5)CC4)CC3)nc2)c2c(C#N)cnn2c1]

### A. Tert-butyl 4-[[4-benzyl-1-[5-(3-cyano-6-ethoxy-pyrazolo[1,5-a]pyridin-4-yl)-2-pyridyl]-4-piperidyl]carbamoyl]piperidine-1-carboxylate

To a solution of 4-[6-(4-amino-4-benzyl-1-piperidyl)-3-pyridyl]-6-ethoxy-pyrazolo[1,5-a]pyridine-3-carbonitrile (150 mg, 0.33 mmol), 1-tert-butoxycarbonylpiperidine-4-carboxylic acid (151.99 mg, 0.66 mmol) in DMA (8 mL) were added HATU (378.09 mg, 0.99 mmol) and DIEA (0.15 mL, 1.99 mmol). The mixture was stirred at rt for 2 h and the crude was purified by reversed phase column chromatography to afford the desired product (155 mg, 70.4% yield) as an off-white solid. MS: m/z [M+H]⁺ 664.

### B. N-[4-benzyl-1-[5-(3-cyano-6-ethoxy-pyrazolo[1,5-a]pyridin-4-yl)-2-pyridyl]-4-piperidyl]piperidine-4-carboxamide

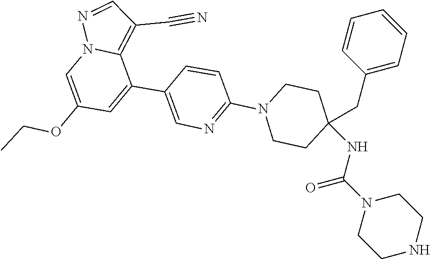 [SMILES: CCOc1cc(-c2ccc(N3CCC(Cc4ccccc4)(NC(=O)N4CCNCC4)CC3)nc2)c2c(C#N)cnn2c1]

To a solution of tert-butyl 4-[[4-benzyl-1-[5-(3-cyano-6-ethoxy-pyrazolo[1,5-a]pyridin-4-yl)-2-pyridyl]-4-piperidyl]carbamoyl]piperidine-1-carboxylate (30 mg, 0.05 mmol) in DCM (1 mL) was added TFA (0.5 mL). The mixture was stirred at rt for 1 h. It was concentrated and purified by Prep-HPLC to afford the titled compound (18.7 mg, 72.6% yield) as a white solid. ¹H NMR (400 MHz, DMSO-d6) δ 8.67 (d, J=2.0 Hz, 1H), 8.58 (s, 1H), 8.32 (d, J=2.5 Hz, 1H), 7.88 (dd, J=9.0, 2.5 Hz, 1H), 7.34-7.28 (m, 3H), 7.27-7.20 (m, 1H), 7.17-7.06 (m, 3H), 4.19-4.13 (m, 4H), 3.35-3.30 (m, 2H), 3.12-2.98 (m, 4H), 2.89-2.82 (m, 2H), 2.48-2.43 (m, 1H), 2.20-2.14 (m, 2H), 1.88-1.78 (m, 4H), 1.63-1.55 (m, 2H), 1.39 (t, J=7.0 Hz, 3H). MS: m/z: [M+H]⁺ 564, purity: 99.3%.

### C. N-[4-benzyl-1-[5-(3-cyano-6-ethoxy-pyrazolo[1,5-a]pyridin-4-yl)-2-pyridyl]-4-piperidyl]-1-[3-[4-[2-(2,6-dioxo-3-piperidyl)-3-oxo-isoindolin-5-yl]-1-piperidyl]propanoyl]piperidine-4-carboxamide

The title compound was prepared in 24.9% yield as a white solid according to the preparation of Example 51 using N-[4-benzyl-1-[5-(3-cyano-6-ethoxy-pyrazolo[1,5-a]pyridin-4-yl)-2-pyridyl]-4-piperidyl]piperidine-4-carboxamide in STEP H. ¹H NMR (400 MHz, DMSO-d6) δ 8.67 (d, J=2.1 Hz, 1H), 8.58 (s, 1H), 8.32 (d, J=2.5 Hz, 1H), 7.88-7.77 (m, 1H), 7.69-7.48 (m, 3H), 7.36-7.20 (m, 4H), 7.14-7.04 (m, 3H), 5.28-4.98 (m, 1H), 4.43-4.41 (m, 2H), 4.31-4.28 (m, 1H), 4.16-4.12 (m, 4H), 3.91-3.88 (m, 1H), 3.67-3.62 (m, 2H), 3.37-3.33 (m, 2H), 3.13-3.11 (m, 3H), 3.08-2.93 (m, 6H), 2.93-2.79 (m, 2H), 2.70-2.51 (m, 2H), 2.46-2.32 (m, 1H), 2.17-2.14 (m, 2H), 2.11-1.89 (m, 5H), 1.82-1.46 (m, 7H), 1.39 (t, J=6.9 Hz, 3H); MS: m/z [M+H]⁺ 945.3, purity: 99.7%; Prep HPLC purification conditions: Column: Xselect CSH C18 OBD Column 30×150 mm 5 μm; Mobile Phase A: Water (0.05% TFA), Mobile Phase B: ACN; Flow rate: 60 mL/min; Gradient: 8% B to 38% B in 10 min, 38% B; RT=10 min.

### Example 53

### N-[4-benzyl-1-[5-(3-cyano-6-ethoxy-pyrazolo[1,5-a]pyridin-4-yl)-2-pyridyl]-4-piperidyl]-2-[4-[3-[4-[2-(2,6-dioxo-3-piperidyl)-3-oxo-isoindolin-5-yl]-1-piperidyl]propanoyl]piperazin-1-yl]acetamide

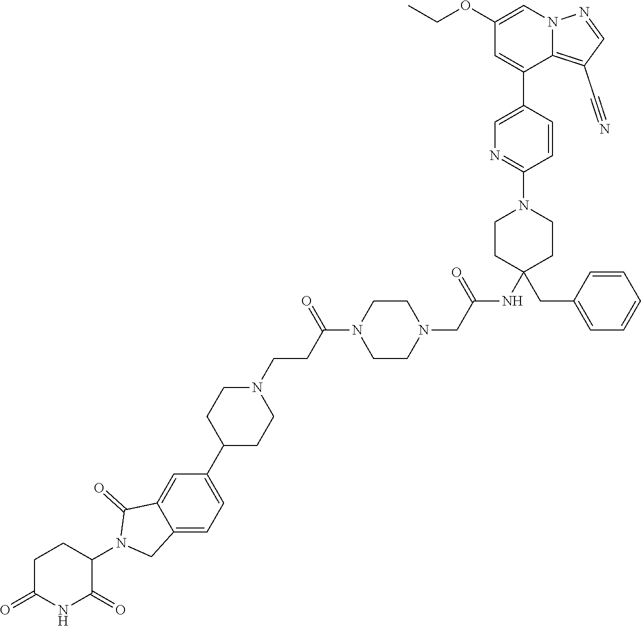 [SMILES: CCOc1cc(-c2ccc(N3CCC(Cc4ccccc4)(NC(=O)CN4CCN(C(=O)CCN5CCC(c6ccc7c(c6)C(=O)N(C6CCC(=O)NC6=O)C7)CC5)CC4)CC3)nc2)c2c(C#N)cnn2c1]

### A. N-[4-benzyl-1-[5-(3-cyano-6-ethoxy-pyrazolo[1,5-a]pyridin-4-yl)-2-pyridyl]-4-piperidyl]-2-piperazin-1-yl-acetamide

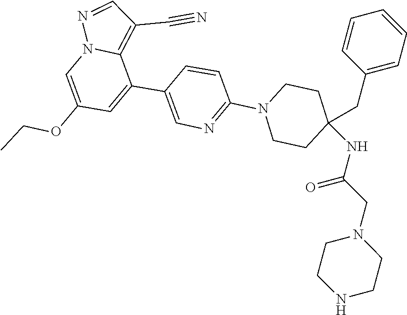 [SMILES: CCOc1cc(-c2ccc(N3CCC(Cc4ccccc4)(NC(=O)CN4CCNCC4)CC3)nc2)c2c(C#N)cnn2c1]

The title compound was prepared in 44.7% overall yield as a white solid according to the preparation of N-[4-benzyl-1-[5-(3-cyano-6-ethoxy-pyrazolo[1,5-a]pyridin-4-yl)-2-pyridyl]-4-piperidyl]piperidine-4-carboxamide (Example 52.B) using 2-(4-tert-butoxycarbonylpiperazin-1-yl)acetic acid in STEP A. ¹H NMR (400 MHz, DMSO-d6) δ 9.04 (s, 1H), 8.67 (d, J=2.5 Hz, 1H), 8.59 (s, 1H), 8.32 (d, J=2.5 Hz, 1H), 7.82-7.69 (m, 2H), 7.35-7.22 (m, 4H), 7.15-7.11 (m, 2H), 7.02 (d, J=9.0 Hz, 1H), 4.21-4.13 (m, 4H), 3.67 (s, 2H), 3.30-3.22 (m, 8H), 3.09-3.03 (m, 4H), 2.18-2.15 (m, 2H), 1.65-1.57 (m, 2H), 1.38 (t, J=7.0 Hz, 3H); MS: m/z: [M+H]⁺ 579, purity: 98.8%.

### B. N-[4-benzyl-1-[5-(3-cyano-6-ethoxy-pyrazolo[1,5-a]pyridin-4-yl)-2-pyridyl]-4-piperidyl]-2-[4-[3-[4-[2-(2,6-dioxo-3-piperidyl)-3-oxo-isoindolin-5-yl]-1-piperidyl]propanoyl]piperazin-1-yl]acetamide

The title compound was prepared in 27.7% yield as a white solid according to the preparation of Example 51 using N-[4-benzyl-1-[5-(3-cyano-6-ethoxy-pyrazolo[1,5-a]pyridin-4-yl)-2-pyridyl]-4-piperidyl]-2-piperazin-1-yl-acetamide in STEP C. ¹H NMR (400 MHz, DMSO-d6) δ 8.66-8.51 (m, 2H), 8.31 (d, J=2.6 Hz, 1H), 7.88-7.65 (m, 1H), 7.57-7.47 (m, 3H), 7.38-7.19 (m, 4H), 7.19-7.07 (m, 2H), 6.95 (d, J=8.9 Hz, 1H), 5.19-4.93 (m, 1H), 4.41-4.39 (m, 1H), 4.28-4.25 (m, 1H), 4.22-4.05 (m, 4H), 3.41-3.39 (m, 3H), 3.19-2.81 (m, 9H), 2.70-2.52 (m, 5H), 2.49-2.30 (m, 7H), 2.17-2.14 (m, 2H), 2.09-1.93 (m, 3H), 1.75-1.73 (m, 2H), 1.69-1.52 (m, 4H), 1.37 (t, J=6.9 Hz, 3H); MS: m/z [M+H]⁺ 960.4, purity: 99.4%. Prep HPLC purification conditions: Column: XBridge Prep OBD C18 Column, 30×150 mm, 5 μm; Mobile Phase A: Water (10 mmol/L NH₄HCO_{3+0.1}% NH₃·H₂O), Mobile Phase B: ACN; Flow rate: 60 mL/min; Gradient: 46% B to 48% B in 9 min, 48% B; RT=8.53 min.

### Example 54

### N-[4-benzyl-1-[5-(3-cyano-6-ethoxy-pyrazolo[1,5-a]pyridin-4-yl)-2-pyridyl]-4-piperidyl]-2-[1-[3-[4-[2-(2,6-dioxo-3-piperidyl)-3-oxo-isoindolin-5-yl]-1-piperidyl]propanoyl]-4-piperidyl]acetamide

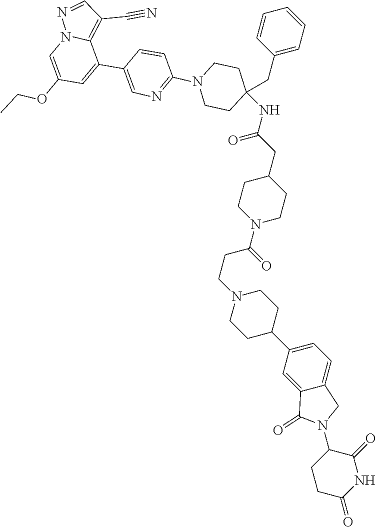 [SMILES: CCOc1cc(-c2ccc(N3CCC(Cc4ccccc4)(NC(=O)CC4CCN(C(=O)CCN5CCC(c6ccc7c(c6)C(=O)N(C6CCC(=O)NC6=O)C7)CC5)CC4)CC3)nc2)c2c(C#N)cnn2c1]

### A. N-[4-benzyl-1-[5-(3-cyano-6-ethoxy-pyrazolo[1,5-a]pyridin-4-yl)-2-pyridyl]-4-piperidyl]-2-(4-piperidyl) acetamide

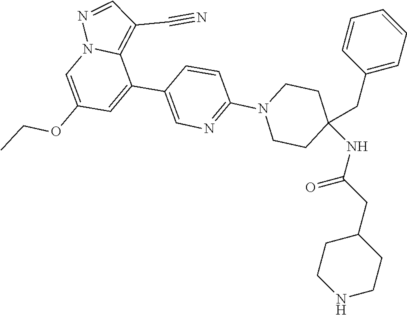 [SMILES: CCOc1cc(-c2ccc(N3CCC(Cc4ccccc4)(NC(=O)CC4CCNCC4)CC3)nc2)c2c(C#N)cnn2c1]

The title compound was prepared in 52.9% overall yield as a white solid according to the preparation of N-[4-benzyl-1-[5-(3-cyano-6-ethoxy-pyrazolo[1,5-a]pyridin-4-yl)-2-pyridyl]-4-piperidyl]piperidine-4-carboxamide (Example 52.B) using 2-(1-tert-butoxycarbonyl-4-piperidyl)acetic acid in STEP A. ¹H NMR (400 MHz, DMSO-d6) δ 8.67 (d, J=2.5 Hz, 1H), 8.58 (s, 1H), 8.31 (d, J=2.5 Hz, 1H), 7.89 (dd, J=9.0, 2.5 Hz, 1H), 7.35-7.20 (m, 4H), 7.16-7.08 (m, 3H), 4.18-4.13 (m, 4H), 3.29-3.22 (m, 2H), 3.16-2.99 (m, 4H), 2.83-2.86 (m, 2H), 2.23-2.09 (m, 4H), 2.07-1.96 (m, 1H), 1.85-1.81 (m, 2H), 1.64-1.52 (m, 2H), 1.41-1.28 (m, 5H); MS: [M+H]⁺ 578.

### B. N-[4-benzyl-1-[5-(3-cyano-6-ethoxy-pyrazolo[1,5-a]pyridin-4-yl)-2-pyridyl]-4-piperidyl]-2-[1-[3-[4-[2-(2,6-dioxo-3-piperidyl)-3-oxo-isoindolin-5-yl]-1-piperidyl]propanoyl]-4-piperidyl]acetamide

The title compound was prepared in 18.1% yield as a white solid according to the preparation of Example 51 using N-[4-benzyl-1-[5-(3-cyano-6-ethoxy-pyrazolo[1,5-a]pyridin-4-yl)-2-pyridyl]-4-piperidyl]-2-(4-piperidyl) acetamide in STEP C. ¹H NMR (400 MHz, DMSO-d6) δ 8.60 (d, J=2.1 Hz, 1H), 8.55 (s, 1H), 8.30 (d, J=2.6 Hz, 1H), 7.79-7.68 (m, 1H), 7.57-7.46 (m, 3H), 7.38-7.18 (m, 5H), 7.15-7.08 (m, 2H), 6.95 (d, J=9.0 Hz, 1H), 5.16-4.97 (m, 1H), 4.55-4.22 (m, 3H), 4.13-4.11 (m, 4H), 3.88-3.85 (m, 1H), 3.57-3.55 (m, 3H), 3.09-2.81 (m, 8H), 2.68-2.52 (m, 4H), 2.42-2.38 (m, 1H), 2.26-1.88 (m, 8H), 1.83-1.60 (m, 6H), 1.59-1.47 (m, 2H), 1.37 (t, J=7.0 Hz, 3H), 1.13-1.10 (m, 1H), 1.06-0.79 (m, 1H); MS: m/z [M+H]⁺ 960.4, purity: 96.8%. Prep HPLC purification conditions: Column: XBridge Prep OBD C18 Column, 30×150 mm, 5 μm; Mobile Phase A: Water (10 mmol/L NH₄HCO_{3+0.1}% NH₃·H₂O), Mobile Phase B: ACN; Flow rate: 60 mL/min; Gradient: 48% B to 50% B in 10 min, 50% B; RT=9.22 min.

### Example 55

### N-[4-benzyl-1-[5-(3-cyano-6-ethoxy-pyrazolo[1,5-apyridin-4-yl)-2-pyridyl]-4-piperidyl]-4-[[4-[2-(2,6-dioxo-3-piperidyl)-3-oxo-isoindolin-5-yl]-1-piperidyl]methyl]piperidine-1-carboxamide

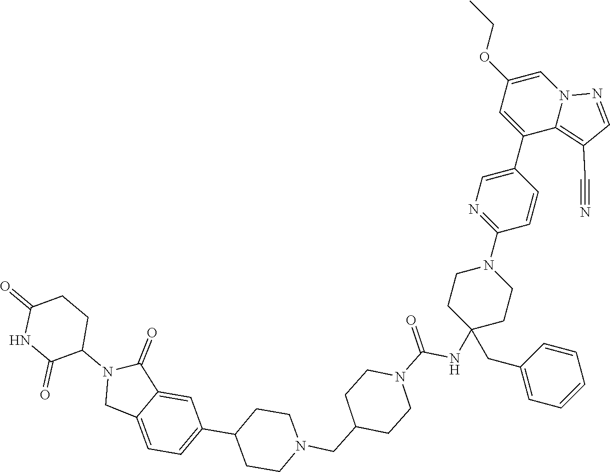 [SMILES: CCOc1cc(-c2ccc(N3CCC(Cc4ccccc4)(NC(=O)N4CCC(CN5CCC(c6ccc7c(c6)C(=O)N(C6CCC(=O)NC6=O)C7)CC5)CC4)CC3)nc2)c2c(C#N)cnn2c1]

### A. Tert-butyl 4-[[4-[2-(2,6-dioxo-3-piperidyl)-3-oxo-isoindolin-5-yl]-1-piperidyl]methyl]piperidine-1-carboxylate

To the mixture solution of 3-[1-oxo-6-(4-piperidyl) isoindolin-2-yl]piperidine-2,6-dione (60 mg, 0.18 mmol) and tert-butyl 4-formylpiperidine-1-carboxylate (80 mg, 0.37 mmol) in DCM (5 mL) was added DIEA (0.03 mL, 0.37 mmol), the mixture was stirred at rt for 5 min, AcOH (0.02 mL, 0.37 mmol) was then added, the mixture was stirred at rt for 0.5 h. The reaction mixture was cooled down to 0 °C, NaBH(OAc)₃ (80 mg, 0.37 mmol) was then added, and the resulting solution was stirred at rt overnight. The reaction was concentrated and purified by Prep-HPLC to afford the desired product (60 mg, 62.4% yield) as white solid. MS: m/z [M+H]⁺ 525.

### B. 3-[1-oxo-6-[1-(4-piperidylmethyl)-4-piperidyl]isoindolin-2-yl]piperidine-2,6-dione

To the mixture solution of tert-butyl 4-[[4-[2-(2,6-dioxo-3-piperidyl)-3-oxo-isoindolin-5-yl]-1-piperidyl]methyl]piperidine-1-carboxylate (55 mg, 0.10 mmol) in DCM (10 mL) was added TFA (2 mL), the mixture solution was stirred at rt for 5 h. The reaction was concentrated to afford the desired product (45 mg, quant.) as an off-white solid, which was used directly in the next step without further purification. MS: m/z [M+H]⁺ 425.

### C. N-[4-benzyl-1-[5-(3-cyano-6-ethoxy-pyrazolo[1,5-apyridin-4-yl)-2-pyridyl]-4-piperidyl]-4-[4-[2-(2,6-dioxo-3-piperidyl)-3-oxo-isoindolin-5-yl]-1-piperidyl]methyl]piperidine-1-carboxamide

To the mixture solution of 4-[6-(4-benzyl-4-isocyanato-1-piperidyl)-3-pyridyl]-6-ethoxy-pyrazolo[1,5-a]pyridine-3-carbonitrile (33.82 mg, 0.07 mmol) in DCM (5 mL) was added 3-[1-oxo-6-[1-(4-piperidylmethyl)-4-piperidyl]isoindolin-2-yl]piperidine-2,6-dione (30 mg, 0.07 mmol) in DMF (3 mL) and DIEA (0.02 mL, 0.21 mmol) at 0 °C, and the mixture solution was stirred at rt for 6 h. The reaction was concentrated and purified by Prep-HPLC to afford the titled product (12.4 mg, 19.2% yield) as a white solid. ¹H NMR (300 MHz, DMSO-d6) δ 10.98 (s, 1H), 8.64 (d, J=2.1 Hz, 1H), 8.57 (s, 1H), 8.31 (d, J=2.6 Hz, 1H), 7.73 (dd, J=8.9, 2.6 Hz, 1H), 7.58-7.49 (m, 3H), 7.34-7.17 (m, 4H), 7.14-7.06 (m, 2H), 6.97-6.90 (m, 1H), 5.40 (s, 1H), 5.11 (dd, J=13.2, 5.1 Hz, 1H), 4.41 (d, J=17.1 Hz, 1H), 4.27 (d, J=17.1 Hz, 1H), 4.20-3.95 (m, 6H), 3.15-3.00 (m, 4H), 3.00-2.83 (m, 3H), 2.72-2.54 (m, 4H), 2.46-2.30 (m, 1H), 2.29-2.12 (m, 4H), 2.07-1.92 (m, 3H), 1.83-1.44 (m, 9H), 1.38 (t, J=7.0 Hz, 3H), 1.06-0.88 (m, 2H); MS: m/z [M+H]⁺ 903.5, purity: 98.6%. Prep-HPLC purification conditions: Column: XBridge Prep OBD C18 Column, 30×150 mm, 5 μm; Mobile Phase A: Water (10 mmol/L NH₄HCO_{3+0.1}% NH₃·H₂O), Mobile Phase B: ACN; Flow rate: 60 mL/min; Gradient: 53% B to 63% B in 9 min, 63% B; RT=8.95 min.

### Example 56

### N-[4-benzyl-1-[5-(3-cyano-6-ethoxy-pyrazolo[1,5-a]pyridin-4-yl)-2-pyridyl]-4-piperidyl]-4-[4-[2-(2,6-dioxo-3-piperidyl)-3-oxo-isoindolin-5-yl]-1-piperidyl]piperidine-1-carboxamide

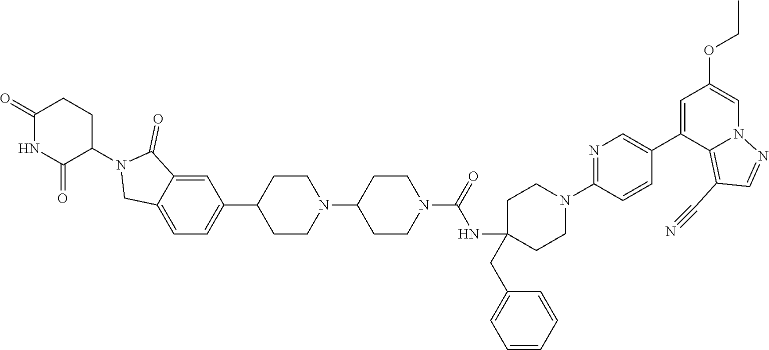 [SMILES: CCOc1cc(-c2ccc(N3CCC(Cc4ccccc4)(NC(=O)N4CCC(N5CCC(c6ccc7c(c6)C(=O)N(C6CCC(=O)NC6=O)C7)CC5)CC4)CC3)nc2)c2c(C#N)cnn2c1]

The title compound was prepared in 41.5% overall yield as a white solid according to the preparation of Example 55 using tert-butyl 4-oxopiperidine-1-carboxylate in STEP A. ¹H NMR (300 MHz, DMSO-d6) δ 11.00 (s, 1H), 9.28 (s, 1H), 8.67 (d, J=2.0 Hz, 1H), 8.58 (s, 1H), 8.31 (d, J=2.6 Hz, 1H), 7.84-7.77 (m, 1H), 7.63-7.55 (m, 2H), 7.55-7.48 (m, 1H), 7.37-7.25 (m, 3H), 7.28-7.17 (m, 1H), 7.16-7.07 (m, 2H), 7.06-6.99 (m, 1H), 5.74 (s, 1H), 5.10 (dd, J=13.2, 5.1 Hz, 1H), 4.44 (d, J=17.3 Hz, 1H), 4.31 (d, J=17.3 Hz, 1H), 4.27-4.08 (m, 6H), 3.66-3.56 (m, 2H), 3.50-3.36 (m, 1H), 3.21-2.81 (m, 8H), 2.78-2.56 (m, 3H), 2.46-2.35 (m, 1H), 2.29-1.86 (m, 9H), 1.63-1.47 (m, 4H), 1.38 (t, J=6.9 Hz, 3H); Analytical LCMS ESI-MS(+) m/z 889.4 [M+H]⁺, RT=1.88 min, purity: 99.6% (Method Q). Prep-HPLC purification conditions: Column: Xselect CSH C18 OBD Column 30×150 mm 5 μm; Mobile Phase A: Water (0.05% TFA), Mobile Phase B: ACN; Flow rate: 60 mL/min; Gradient: 4% B to 34% B in 10 min, 34% B; RT=10.93 min.

### Example 57

### N-[4-benzyl-1-[5-(3-cyano-6-ethoxy-pyrazolo[1,5-a]pyridin-4-yl)-2-pyridyl]-4-piperidyl]-2-[4-[[4-[2-(2,6-dioxo-3-piperidyl)-3-oxo-isoindolin-5-yl]-1-piperidyl]methyl]-1-piperidyl]acetamide

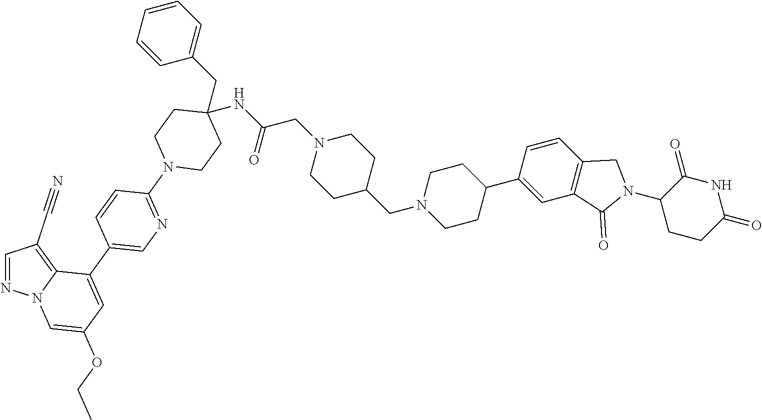 [SMILES: CCOc1cc(-c2ccc(N3CCC(Cc4ccccc4)(NC(=O)CN4CCC(CN5CCC(c6ccc7c(c6)C(=O)N(C6CCC(=O)NC6=O)C7)CC5)CC4)CC3)nc2)c2c(C#N)cnn2c1]

### A. Ethyl 1-(2-tert-butoxy-2-oxo-ethyl)piperidine-4-carboxylate

To the mixture solution of ethyl piperidine-4-carboxylate (500 mg, 3.18 mmol) and tert-butyl 2-bromoacetate (620.35 mg, 3.18 mmol) in DCM (30 mL) was added Et₃N (321.23 mg, 3.18 mmol), the mixture was stirred at 40 °C for overnight. The reaction was concentrated and purified by Prep-HPLC to afford the desired product (800 mg, 92.7% yield) as a colorless oil. MS: m/z [M+H]⁺ 272.

### B. Ethoxycarbonyl-1-piperidyl)acetic acid

To the mixture solution of ethyl 1-(2-tert-butoxy-2-oxo-ethyl)piperidine-4-carboxylate (800 mg, 2.95 mmol) in DCM (20 mL) was added TFA (4 mL). It was stirred at rt for 4 h, the reaction was concentrated to afford 2-(4-ethoxycarbonyl-1-piperidyl)acetic acid (600 mg, 94.5% yield) as a yellow solid. MS: m/z [M+H]⁺ 216.

### C. Ethyl 1-[2-[[4-benzyl-1-[5-(3-cyano-6-ethoxy-pyrazolo[1,5-a]pyridin-4-yl)-2-pyridyl]-4-piperidyl]amino]-2-oxo-ethyl]piperidine-4-carboxylate

To the mixture solution of 2-(4-ethoxycarbonyl-1-piperidyl)acetic acid (142.69 mg, 0.66 mmol), 4-[6-(4-amino-4-benzyl-1-piperidyl)-3-pyridyl]-6-ethoxy-pyrazolo[1,5-a]pyridine-3-carbonitrile (200 mg, 0.44 mmol), and HATU (0.17 mL, 0.66 mmol) in DMF (6 mL) was added DIEA (171.03 mg, 1.33 mmol) at 0 °C, the mixture solution was stirred at −5 °C for 1 h. The reaction was diluted with water, extracted with EA, washed with brine, dried, and purified by silica gel column chromatography to afford the desired product (250 mg, 87.1% yield) as a white solid. MS: m/z [M+H]⁺ 650.

### D. N-[4-benzyl-1-[5-(3-cyano-6-ethoxy-pyrazolo[1,5-a]pyridin-4-yl)-2-pyridyl]-4-piperidyl]-2-[4-(hydroxymethyl)-1-piperidyl]acetamide

To the mixture solution of ethyl 1-[2-[[4-benzyl-1-[5-(3-cyano-6-ethoxy-pyrazolo[1,5-a]pyridin-4-yl)-2-pyridyl]-4-piperidyl]amino]-2-oxo-ethyl]piperidine-4-carboxylate (150 mg, 0.23 mmol) in THF (10 mL) was added LiBH₄ (0.3 mL, 2 mmol/L, 0.6 mmol) at 0 °C, the mixture solution was stirred at 35 °C for 5 h. The reaction was quenched by dropwise addition of 10% citric acid. The resulting mixture was extracted with EtOAc, the combined organic layers were dried over sodium sulfate, concentrated in vacuo. The residue was purified by silica gel column to afford the desired product (70 mg, 49.9% yield) as a white solid. MS: m/z [M+H]⁺ 608.

### E. N-[4-benzyl-1-[5-(3-cyano-6-ethoxy-pyrazolo[1,5-a]pyridin-4-yl)-2-pyridyl]-4-piperidyl]-2-(4-formyl-1-piperidyl) acetamide

To the mixture solution of N-[4-benzyl-1-[5-(3-cyano-6-ethoxy-pyrazolo[1,5-a]pyridin-4-yl)-2-pyridyl]-4-piperidyl]-2-[4-(hydroxymethyl)-1-piperidyl]acetamide (70 mg, 0.12 mmol) in DMSO (5 mL) was added IBX (64.5 mg, 0.23 mmol), the mixture solution was stirred at 70 °C for 3 h. The reaction was diluted with water, extracted with EtOAc (5×), dried, concentrated to afford the desired product (20 mg, 20.1% yield) as a yellow solid. The crude was used without further purification in the next step. MS: m/z [M+H]⁺ 606.

### F. N-[4-benzyl-1-[5-(3-cyano-6-ethoxy-pyrazolo[1,5-a]pyridin-4-yl)-2-pyridyl]-4-piperidyl]-2-[4-[[4-[2-(2,6-dioxo-3-piperidyl)-3-oxo-isoindolin-5-yl]-1-piperidyl]methyl]-1-piperidyl]acetamide

To the mixture solution of N-[4-benzyl-1-[5-(3-cyano-6-ethoxy-pyrazolo[1,5-a]pyridin-4-yl)-2-pyridyl]-4-piperidyl]-2-(4-formyl-1-piperidyl) acetamide (17 mg, 0.03 mmol) and 3-[1-oxo-6-(4-piperidyl) isoindolin-2-yl]piperidine-2,6-dione (13.78 mg, 0.04 mmol) in DCM (5 mL) was added DIEA (0.15 mL, 0.06 mmol), the mixture solution was stirred at rt for 5 minutes, and then Ti(OiPr)₄ (15.94 mg, 0.06 mol) was added, the mixture was stirred at rt for 0.5 h, then NaBH(OAc)₃ (11.9 mg, 0.06 mmol) was added at 0 °C, the mixture solution was stirred at rt overnight. The reaction was concentrated and purified by Prep-HPLC to afford the titled compound (6.4 mg, 24.1% yield) as a white solid. ¹H NMR (300 MHz, DMSO-d6) δ 10.99 (s, 1H), 8.65 (d, J=2.1 Hz, 1H), 8.57 (s, 1H), 8.32 (d, J=2.6 Hz, 1H), 7.76 (dd, J=8.8, 2.6 Hz, 1H), 7.59-7.49 (m, 3H), 7.36-7.20 (m, 4H), 7.18-7.09 (m, 3H), 6.97 (d, J=8.8 Hz, 1H), 5.10 (dd, J=13.1, 4.9 Hz, 1H), 4.48-4.08 (m, 8H), 3.08-2.73 (m, 11H), 2.69-2.55 (m, 2H), 2.45-2.33 (m, 1H), 2.21-2.07 (m, 5H), 2.06-1.91 (m, 3H), 1.81-1.54 (m, 8H), 1.38 (t, J=6.9 Hz, 3H), 1.25-1.05 (m, 2H); MS: m/z [M+H]⁺ 917.4, purity: 97.1%. Prep HPLC purification conditions: Column: XBridge Prep OBD C18 Column, 30×150 mm, 5 μm; Mobile Phase A: Water (10 mmol/L NH₄HCO_{3+0.1}% NH₃·H₂O), Mobile Phase B: ACN; Flow rate: 60 mL/min; Gradient: 62% B to 75% B in 8 min, 75% B; RT=6.05 min.

### Example 58

### N-(1-(5-(3-cyano-6-ethoxypyrazolo[1,5-a]pyridin-4-yl)pyridin-2-yl)-4-((4-(3-(4-(4-((2,6-dioxopiperidin-3-yl)amino)phenyl)piperazin-1-yl)propanoyl)piperazin-1-yl)methyl)piperidin-4-yl)-2,5-difluorobenzamide

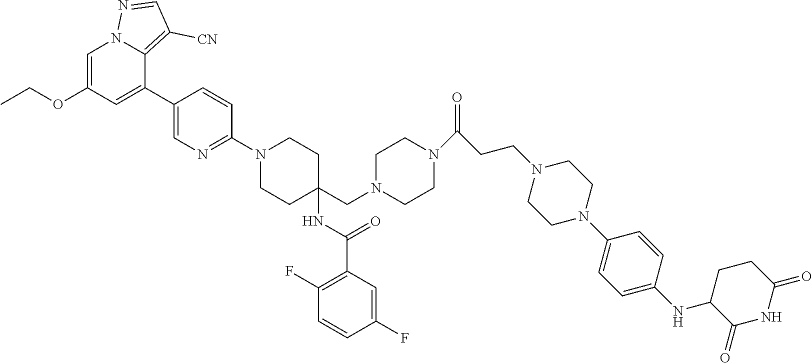 [SMILES: CCOc1cc(-c2ccc(N3CCC(CN4CCN(C(=O)CCN5CCN(c6ccc(NC7CCC(=O)NC7=O)cc6)CC5)CC4)(NC(=O)c4cc(F)ccc4F)CC3)nc2)c2c(C#N)cnn2c1]

### A. Tert-butyl 4-(4-((2,6-dioxopiperidin-3-yl)amino)phenyl)piperazine-1-carboxylate

To a solution of tert-butyl 4-(4-aminophenyl)piperazine-1-carboxylate (1 g, 3.61 mmol), 3-bromopiperidine-2,6-dione (1.38 g, 7.21 mmol) in DMF (20 mL) was added DIEA (1.94 mL, 10.82 mmol). The mixture was stirred at 80 °C for 40 h. After cooling to rt, water was added to quench the reaction and extracted with EA, the combined organic layers were washed with brine, dried over sodium sulfate, filtered and concentrated. The residue was purified by column chromatography to afford the desired product (1 g, 71.4% yield) as a yellow solid. MS: m/z [M+H]⁺ 389.

### B. 3-((4-(piperazin-1-yl)phenyl)amino)piperidine-2,6-dione

To a solution of tert-butyl 4-[4-[(2,6-dioxo-3-piperidyl)amino]phenyl]piperazine-1-carboxylate (1 g, 2.57 mmol) in 1,4-dioxane (20 mL) was added HCl (4M in dioxane) (20 mL). The mixture was stirred at rt for 4 h. It was filtered, the filter cake was dried to afford the desired product (700 mg, 94.3% yield) as a white solid. MS: m/z: [M+H]⁺ 289.

### C. Tert-butyl 3-(4-(4-((2,6-dioxopiperidin-3-yl)amino)phenyl)piperazin-1-yl)propanoate

To a solution of 3-(4-piperazin-1-ylanilino)piperidine-2,6-dione (100 mg, 0.35 mmol) and tert-butyl prop-2-enoate (88.9 mg, 0.69 mmol) in MeCN (5 mL) was added DBU (160.48 mg, 1.04 mmol). The mixture was stirred at rt for 16 h. The solvent was concentrated, and the residue was added water and extracted with EtOAc (3×). The organic layers were dried over sodium sulfate, filtered and concentrated. The residue was purified by reversed column chromatography to afford the desired product (100 mg, 69.2% yield) as a yellow oil. MS: m/z [M+H]⁺ 417.

### D. 3-(4-(4-((2,6-dioxopiperidin-3-yl)amino)phenyl)piperazin-1-yl)propanoic acid

To a solution of tert-butyl 3-[4-[4-[(2,6-dioxo-3-piperidyl)amino]phenyl]piperazin-1-yl]propanoate (95 mg, 0.23 mmol) in 1,4-Dioxane (2 mL) was added HCl (4M in dioxane) (2 mL). The mixture was stirred at rt for 2 h. The mixture was concentrated to afford crude product which was used directly in the next step without further purification. MS: m/z [M+H]⁺ 361.

### E. N-(1-(5-(3-cyano-6-ethoxypyrazolo[1,5-a]pyridin-4-yl)pyridin-2-yl)-4-((4-(3-(4-(4-((2,6-dioxopiperidin-3-yl)amino)phenyl)piperazin-1-yl)propanoyl)piperazin-1-yl)methyl)piperidin-4-yl)-2,5-difluorobenzamide

To the mixture solution of 3-[4-[4-[(2,6-dioxo-3-piperidyl)amino]phenyl]piperazin-1-yl]propanoic acid (30 mg, 0.08 mmol), N-[1-[5-(3-cyano-6-ethoxy-pyrazolo[1,5-a]pyridin-4-yl)-2-pyridyl]-4-(piperazin-1-ylmethyl)-4-piperidyl]-2,5-difluoro-benzamide (50 mg, 0.08 mmol) and HATU (50 mg, 0.12 mmol) in DMF (3 mL) was added DIEA (0.02 mL, 0.25 mmol) at −5 °C, the mixture solution was stirred at −5 °C for 2 h. The reaction was purified by Prep-HPLC to afford the titled compound (37.2 mg, 45.9% yield) as a grey solid. ¹H NMR (400 MHz, DMSO-d6) δ 10.81 (s, 1H), 9.74 (s, 1H), 8.69-8.60 (m, 3H), 8.38 (s, 1H), 7.86-7.84 (m, 1H), 7.54-7.38 (m, 3H), 7.30 (s, 1H), 7.10-7.08 (m, 1H), 6.85 (d, J=8.3 Hz, 2H), 6.68 (d, J=8.3 Hz, 2H), 4.33-4.22 (m, 4H), 4.22-4.12 (m, 3H), 3.66-3.53 (m, 7H), 3.45-3.35 (m, 3H), 3.29-3.16 (m, 6H), 2.98-2.83 (m, 5H), 2.80-2.62 (m, 2H), 2.64-2.56 (m, 1H), 1.92-1.82 (d, m, 2H), 1.93-1.68 (m, 4H), 1.39 (d, J=7.2 Hz, 3H); MS: m/z [M+H]⁺ 943.4, purity: 97.5%. Prep HPLC purification conditions: Column: SunFire Prep C18 OBD Column, 19×150 mm, 5 μm; Mobile Phase A: water (0.05% TFA), Mobile Phase B: ACN; Flow rate: 20 mL/min; Gradient: 30% B to 50% B in 6 min, 50% B; RT=5.53 min.

### Example 59

### N-(1-(5-(3-cyano-6-ethoxypyrazolo[1,5-a]pyridin-4-yl)pyridin-2-yl)-4-((4-(3-(4-(3-((2,6-dioxopiperidin-3-yl)amino)phenyl)piperazin-1-yl)propanoyl)piperazin-1-yl)methyl)piperidin-4-yl)-2,5-difluorobenzamide

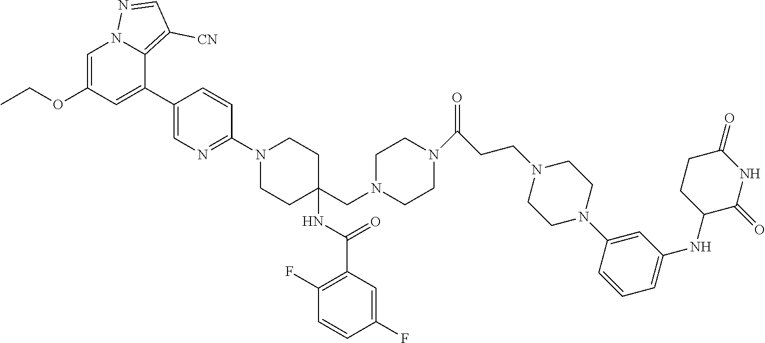 [SMILES: CCOc1cc(-c2ccc(N3CCC(CN4CCN(C(=O)CCN5CCN(c6cccc(NC7CCC(=O)NC7=O)c6)CC5)CC4)(NC(=O)c4cc(F)ccc4F)CC3)nc2)c2c(C#N)cnn2c1]

The title compound was prepared in 12.7% overall yield as an off white solid according to the preparation of Example 58 using tert-butyl 4-(3-aminophenyl)piperazine-1-carboxylate in STEP A. ¹H NMR (400 MHz, DMSO-d6) δ 10.81 (s, 1H), 9.62 (brs, 1H), 8.69-8.60 (m, 3H), 8.37 (s, 1H), 7.84-7.82 (m, 1H), 7.49-7.36 (m, 3H), 7.29 (s, 1H), 7.08-7.06 (m, 1H), 7.00-6.96 (m, 1H), 6.32 (s, 1H), 6.27-6.23 (m, 2H), 4.35-4.26 (m, 4H), 4.1-4.16 (m, 3H), 3.77-3.73 (m, 3H), 3.63-3.59 (m, 4H), 3.45-3.35 (m, 3H), 3.28-3.12 (m, 7H), 3.00-2.84 (m, 5H), 2.81-2.65 (m, 3H), 2.42-2.40 (m, 1H), 2.14-2.05 (m, 1H), 1.93-1.83 (m, 1H), 1.78-1.68 (m, 3H), 1.39 (d, J=7.0 Hz, 3H); Analytical LCMS ESI-MS(+) m/z 943.5 [M+H]⁺, RT=1.29 min, purity: 100% (Method Q). Prep HPLC purification conditions: Column: SunFire Prep C18 OBD Column, 19×150 mm, 5 μm; Mobile Phase A: water (0.5% TFA), Mobile Phase B: ACN; Flow rate: 20 mL/min; Gradient: 35% B to 45% B in 6 min, 45% B; RT=5.63 min.

### Example 60

### N-(1-(5-(3-cyano-6-ethoxypyrazolo[1,5-a]pyridin-4-yl)pyridin-2-yl)-4-((4-(3-(4-(4-(2,6-dioxopiperidin-3-yl)phenyl)piperazin-1-yl)propanoyl)piperazin-1-yl)methyl)piperidin-4-yl)-2,5-difluorobenzamide

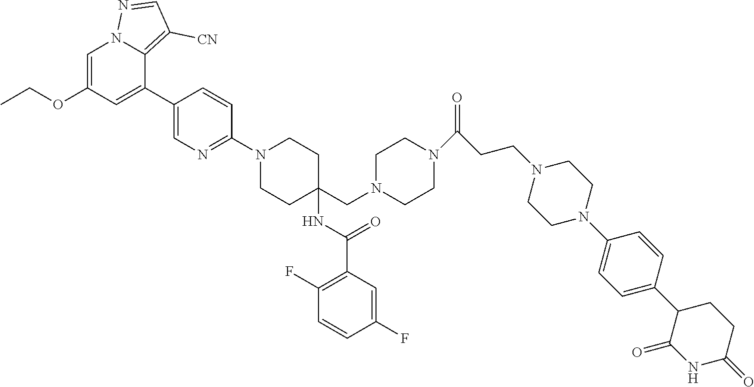 [SMILES: CCOc1cc(-c2ccc(N3CCC(CN4CCN(C(=O)CCN5CCN(c6ccc(C7CCC(=O)NC7=O)cc6)CC5)CC4)(NC(=O)c4cc(F)ccc4F)CC3)nc2)c2c(C#N)cnn2c1]

The title compound was prepared in 9.1% overall yield as a white solid according to the preparation of Example 58 using 3-(4-(piperazin-1-yl)phenyl)piperidine-2,6-dione in STEP C. ¹H NMR (400 MHz, DMSO-d6) δ 10.80 (s, 1H), 8.67 (s, 1H), 8.59 (s, 1H), 8.34 (d, J=2.6 Hz, 1H), 8.18 (s, 1H), 7.77 (dd, J=8.9, 2.6 Hz, 1H), 7.38-7.33 (m, 3H), 7.29 (d, J=2.6 Hz, 1H), 7.07-6.98 (m, 3H), 6.90 (d, J=8.9 Hz, 2H), 4.23-4.14 (m, 4H), 3.75-3.71 (m, 1H), 3.47-3.43 (m, 3H), 3.39-3.36 (m, 4H), 3.19-3.09 (m, 6H), 2.76-2.73 (m, 2H), 2.57-2.54 (m, 5H), 2.50-2.48 (m, 5H), 2.37-2.33 (m, 3H), 2.19-2.09 (m, 1H), 2.05-1.91 (m, 1H), 1.71-1.53 (m, 2H), 1.39 (t, J=6.9 Hz, 3H); Analytical LCMS ESI-MS(+) m/z 465.1 [M+2H]²⁺, RT=1.48 min, purity: 98.4% (Method Q). Prep HPLC purification conditions: Column: YMC-Actus Triart C18 E×RS, 30×250 mm, 5 μm; Mobile Phase A: Water (10 mmol/L NH₄HCO_{3+0.1}% NH₃·H₂O), Mobile Phase B: ACN; Flow rate: 60 mL/min; Gradient: 50% B to 60% B in 8 min, 60% B; RT=5.25 min.

### Example 61

### N-(1-(5-(3-cyano-6-ethoxypyrazolo[1,5-a]pyridin-4-yl)pyridin-2-yl)-4-((4-(3-(4-(3-(2,6-dioxopiperidin-3-yl)phenyl)piperazin-1-yl)propanoyl)piperazin-1-yl)methyl)piperidin-4-yl)-2,5-difluorobenzamide

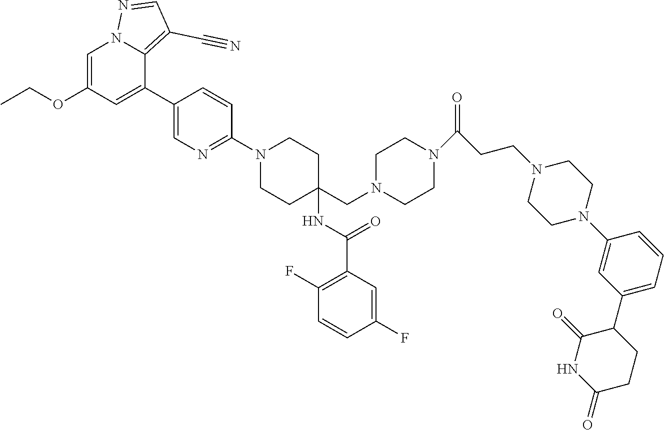 [SMILES: CCOc1cc(-c2ccc(N3CCC(CN4CCN(C(=O)CCN5CCN(c6cccc(C7CCC(=O)NC7=O)c6)CC5)CC4)(NC(=O)c4cc(F)ccc4F)CC3)nc2)c2c(C#N)cnn2c1]

### A. Tert-butyl 4-(3-bromophenyl)-4-cyanobutanoate

To a solution of 2-(3-bromophenyl) acetonitrile (2 g, 10.2 mmol), tert-butyl prop-2-enoate (1.31 g, 10.2 mmol) in Toluene (20 mL) were added K₂CO₃ (1.41 g, 10.2 mmol) and BTEAC (231.59 mg, 1.02 mmol). The mixture was stirred at 75 °C for 16 h. The reaction mixture was concentrated and purified by reversed column chromatography to afford the desired product (1.25 g, 37.8% yield) as a yellow oil. MS: m/z [M+H]⁺ 324.

### B. Tert-butyl 4-(3-(4-(tert-butoxy)-1-cyano-4-oxobutyl)phenyl)piperazine-1-carboxylate

To a 100 mL round bottle flask, tert-butyl 4-(3-bromophenyl)-4-cyanobutanoate (1.24 g, 3.82 mmol), tert-butyl piperazine-1-carboxylate (854.82 mg, 4.59 mmol), Pd₂(dba)₃ (174.98 mg, 0.19 mmol), Xphos (127.71 mg, 0.27 mmol) and Cs₂CO₃ (2.49 g, 7.65 mmol) were suspended in 1,4-Dioxane (30 mL). The mixture was placed under vacuum, sonicated and backfilled with nitrogen. The mixture was stirred at 90 °C for 16 h. It was filtered, concentrated, the crude was purified by column chromatography (PE:EA=1:10 to PE:EA-1:1) to afford the desired product (1.3 g, 79.1% yield) as a yellow solid. MS: m/z 471 [M+H+41]⁺.

### C. 3-(3-(piperazin-1-yl)phenyl)piperidine-2,6-dione

To a solution of tert-butyl 4-[3-(4-tert-butoxy-1-cyano-4-oxo-butyl)phenyl]piperazine-1-carboxylate (1.3 g, 3.03 mmol) in DCE (20 mL) was added MsOH (872.61 mg, 9.08 mmol), Tf₂O (0.16 mL, 0.92 mmol). The mixture was stirred at 90 °C for 4 h. It was concentrated was used directly in the next step without further purification. MS: m/z [M+H]⁺ 274.

### D. Tert-butyl 4-(3-(2,6-dioxopiperidin-3-yl)phenyl)piperazine-1-carboxylate

To a solution of 3-(3-piperazin-1-ylphenyl)piperidine-2,6-dione (1.5 g, 5.49 mmol) in DMF (30 mL) were added (Boc) 2O (3.78 mL, 16.46 mmol), K₂CO₃ (2271.98 mg, 16.46 mmol). The mixture was stirred at rt for 4 h. Water was used to quench the reaction. Extracted with EA, the organic phase was washed with brine, combined the organic phase, dried, filtered, concentrated, the crude was purified by column chromatography to afford the desired product (1 g, 48.8% yield) as a yellow solid. MS: m/z [M+H]⁺ 374.

### E. 3-(3-(piperazin-1-yl)phenyl)piperidine-2,6-dione

To a solution of tert-butyl 4-[3-(2,6-dioxo-3-piperidyl)phenyl]piperazine-1-carboxylate (1 g, 2.68 mmol) in 1,4-Dioxane (10 mL) was added HCl (4M in 1,4-dioxane) (10 mL). The mixture was stirred at rt for 2 h. It was filtered and dried under vacuum to afford the crude product (700 mg, 95.6% yield), which was used directly in the next step without further purification. MS: m/z [M+H]⁺ 274.

### F. Tert-butyl 3-(4-(3-(2,6-dioxopiperidin-3-yl)phenyl)piperazin-1-yl)propanoate

To a solution of 3-(3-piperazin-1-ylphenyl)piperidine-2,6-dione (100 mg, 0.37 mmol), tert-butyl prop-2-enoate (93.78 mg, 0.73 mmol) in MeCN (5 mL) was added DBU (167.09 mg, 1.1 mmol). The mixture was stirred at rt for 16 h. It was concentrated and purified by reversed phase column chromatography to afford the desired product (90 mg, 61.3% yield) as a yellow oil. MS: m/z [M+H]⁺ 402.

### G. 3-(4-(3-(2,6-dioxopiperidin-3-yl)phenyl)piperazin-1-yl)propanoic acid

To a solution of tert-butyl 3-[4-[3-(2,6-dioxo-3-piperidyl)phenyl]piperazin-1-yl]propanoate (85 mg, 0.21 mmol) in DCM (3 mL) was added TFA (3 mL). The mixture was stirred at rt for 2 h. the reaction mixture was concentrated to afford crude product, which was used directly in the next step without further purification. MS: m/z [M+H]⁺ 346.

### H. N-(1-(5-(3-cyano-6-ethoxypyrazolo[1,5-a]pyridin-4-yl)pyridin-2-yl)-4-((4-(3-(4-(3-(2,6-dioxopiperidin-3-yl)phenyl)piperazin-1-yl)propanoyl)piperazin-1-yl)methyl)piperidin-4-yl)-2,5-difluorobenzamide

To a solution of N-[1-[5-(3-cyano-6-ethoxy-pyrazolo[1,5-a]pyridin-4-yl)-2-pyridyl]-4-(piperazin-1-ylmethyl)-4-piperidyl]-2,5-difluoro-benzamide (50 mg, 0.08 mmol) in DMA (3 mL) were added 3-[4-[3-(2,6-dioxo-3-piperidyl)phenyl]piperazin-1-yl]propanoic acid (28.75 mg, 0.08 mmol), HATU (47.45 mg, 0.12 mmol) and DIEA (32.21 mg, 0.25 mmol). The mixture was stirred at rt for 4 h and purified by prep-HPLC to afford the titled product (24.7 mg, 30.7% yield) as a white solid. ¹H NMR (400 MHz, DMSO-d6) δ 10.84 (s, 1H), 9.88 (s, 1H), 8.70-8.63 (m, 1H), 8.60 (s, 1H), 8.37 (d, J=2.4 Hz, 1H), 7.89-7.85 (m, 1H), 7.53-7.48 (m, 1H), 7.46-7.35 (m, 2H), 7.30 (d, J=2.4 Hz, 1H), 7.23 (t, J=7.8 Hz, 1H), 7.12 (d, J=8.9 Hz, 1H), 6.96-6.87 (m, 2H), 6.73 (d, J=7.8 Hz, 1H), 4.31-4.26 (m, 2H), 4.20-4.14 (m, 2H), 3.92-3.76 (m, 3H), 3.67-3.61 (m, 4H), 3.44-3.40 (m, 2H), 3.35-3.12 (m, 5H), 3.04-2.92 (m, 4H), 2.72-2.62 (m, 1H), 2.52-2.50 (m, 8H), 2.46-2.44 (m, 2H), 2.28-2.17 (m, 1H), 2.10-1.93 (m, 1H), 1.84-1.67 (m, 2H), 1.39 (t, J=6.9 Hz, 3H); MS: m/z: [M+H]⁺ 464.9, purity: 96.3%. Prep HPLC purification conditions: Column: SunFire Prep C18 OBD Column, 19×150 mm, 5 μm; Mobile Phase A: Water (0.05% TFA), Mobile Phase B: ACN; Flow rate: 20 mL/min; Gradient: 35% B to 50% B in 5.1 min, 50% B; RT=5.33 min. Using procedures like Examples 1-14, the following Examples 62-66 were obtained.

### Example 62

### N-[1-(5-{3-cyano-6-ethoxypyrazolo[1,5-a]pyridin-4-yl}pyridin-2-yl)-4-{[4-(1-{2-[2-(2,6-dioxopiperidin-3-yl)-1-oxo-2,3-dihydro-1H-isoindol-5-yl]acetyl}azetidin-3-yl)piperazin-1-yl]methyl}piperidin-4-yl]-2,5-difluorobenzamide

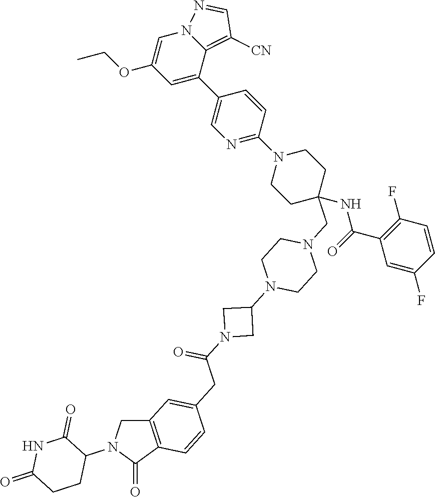 [SMILES: CCOc1cc(-c2ccc(N3CCC(CN4CCN(C5CN(C(=O)Cc6ccc7c(c6)CN(C6CCC(=O)NC6=O)C7=O)C5)CC4)(NC(=O)c4cc(F)ccc4F)CC3)nc2)c2c(C#N)cnn2c1]

### Example 63

### N-[1-(5-{3-cyano-6-ethoxypyrazolo[1,5-a]pyridin-4-yl}pyridin-2-yl)-4-[(4-{1-[2-(2,6-dioxopiperidin-3-yl)-1-oxo-2,3-dihydro-1H-isoindole-5-carbonyl]azetidin-3-yl}piperazin-1-yl)methyl]piperidin-4-yl]-2,5-difluorobenzamide

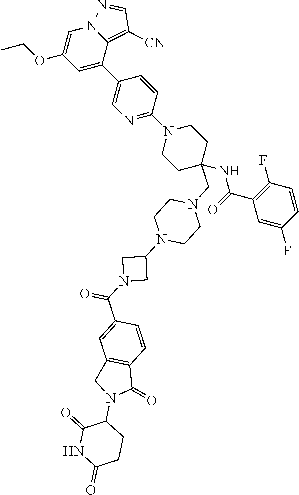 [SMILES: CCOc1cc(-c2ccc(N3CCC(CN4CCN(C5CN(C(=O)c6ccc7c(c6)CN(C6CCC(=O)NC6=O)C7=O)C5)CC4)(NC(=O)c4cc(F)ccc4F)CC3)nc2)c2c(C#N)cnn2c1]

### Example 64

### N-[1-(5-{3-cyano-6-ethoxypyrazolo[1,5-a]pyridin-4-yl}pyridin-2-yl)-4-[(4-{1-[3-(2,4-dioxo-1,3-diazinan-1-yl)-1-methyl-1H-indazole-6-carbonyl]azetidin-3-yl}piperazin-1-yl)methyl]piperidin-4-yl]-2,5-difluorobenzamide

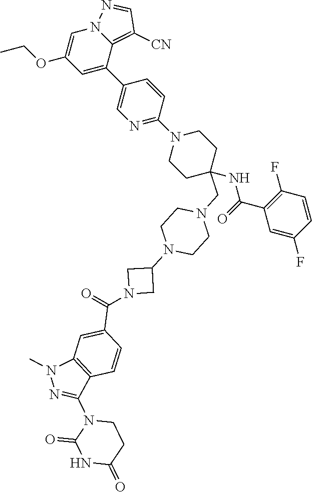 [SMILES: CCOc1cc(-c2ccc(N3CCC(CN4CCN(C5CN(C(=O)c6ccc7c(N8CCC(=O)NC8=O)nn(C)c7c6)C5)CC4)(NC(=O)c4cc(F)ccc4F)CC3)nc2)c2c(C#N)cnn2c1]

### Example 65

### N-[1-(5-{3-cyano-6-ethoxypyrazolo[1,5-a]pyridin-4-yl}pyridin-2-yl)-4-({4-[1-(2-{[2-(2,6-dioxopiperidin-3-yl)-1-oxo-2,3-dihydro-1H-isoindol-5-yl]oxy}acetyl)azetidin-3-yl]piperazin-1-yl}methyl)piperidin-4-yl]-2,5-difluorobenzamide

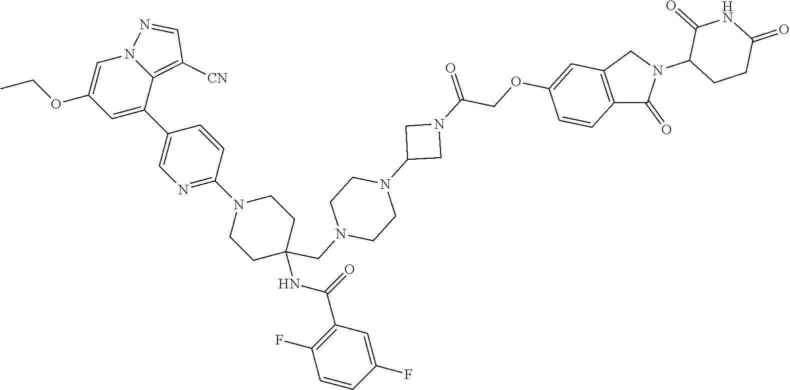 [SMILES: CCOc1cc(-c2ccc(N3CCC(CN4CCN(C5CN(C(=O)COc6ccc7c(c6)CN(C6CCC(=O)NC6=O)C7=O)C5)CC4)(NC(=O)c4cc(F)ccc4F)CC3)nc2)c2c(C#N)cnn2c1]

### Example 66

### N-[4-benzyl-1-(5-{3-cyano-6-ethoxypyrazolo[1,5-a]pyridin-4-yl}pyridin-2-yl)piperidin-4-yl]-1-(3-{4-[2-(2,6-dioxopiperidin-3-yl)-1-oxo-2,3-dihydro-1H-isoindol-5-yl]piperidin-1-yl}propanoyl)piperidine-4-carboxamide

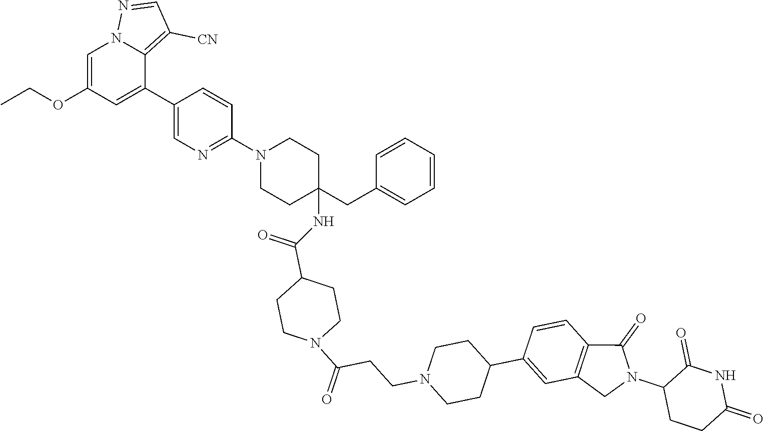 [SMILES: CCOc1cc(-c2ccc(N3CCC(Cc4ccccc4)(NC(=O)C4CCN(C(=O)CCN5CCC(c6ccc7c(c6)CN(C6CCC(=O)NC6=O)C7=O)CC5)CC4)CC3)nc2)c2c(C#N)cnn2c1]

| | Analytical | |
|---|---|---|
| | LCMS | |
| | ESI-MS(+) | |
| | m/z/RT | |
| | (min) | |
| Example # | (Method); | ¹H NMR |
| 62 | 940.2 | ¹H NMR (500 MHz, DMSO-d₆) δ 10.98 (s, 1H), 8.63 (s, 1H), |
| | [M + H]⁺/ | 8.56 (s, 1H), 8.32 (d, J = 2.0 Hz, 1H), 8.09 (s, 1H), 7.75 (dd, |
| | 1.49 min | J = 8.6, 2.3 Hz, 1H), 7.66 (d, J = 7.8 Hz, 1H), 7.46 (s, 1H), 7.39- |
| | (A) | 7.30 (m, 4H), 7.26 (d, J = 1.8 Hz, 1H), 6.97 (d, J = 9.1 Hz, |
| | | 1H), 5.10 (dd, J = 13.2, 5.0 Hz, 1H), 4.44 (br d, J = 17.4 Hz, |
| | | 1H), 4.31 (d, J = 17.4 Hz, 1H), 4.23-4.12 (m, 5H), 4.05-3.99 |
| | | (m, 1H), 3.84 (br t, J = 8.0 Hz, 1H), 3.69-3.57 (m, 1H), 3.55- |
| | | 3.35 (m, 3H), 3.16 (br t, J = 11.9 Hz, 2H), 3.11-3.03 (m, 1H), |
| | | 2.91 (br s, 1H), 2.72 (s, 2H), 2.61 (br d, J = 19.1 Hz, 2H), 2.55 |
| | | (DMSO), 2.44-2.23 (m, 7H), 2.05-1.97 (m, 1H), 1.65-1.55 |
| | | (m, 2H), 1.38 (t, J = 6.9 Hz, 3H) ppm |
| 63 | 926.2 | ¹H NMR (500 MHz, DMSO-d₆) δ 8.63 (d, J = 1.5 Hz, 1H), |
| | [M + H]⁺/ | 8.56 (s, 1H), 8.32 (d, J = 2.4 Hz, 1H), 8.09 (s, 1H), 7.90 (d, |
| | 1.93 min | J = 6.9 Hz, 2H), 7.89 (s, 1H), 7.75 (dd, J = 8.8, 2.4 Hz, 1H), |
| | (A); | 7.70 (d, J = 8.3 Hz, 1H), 7.39-7.29 (m, 3H), 7.26 (d, J = 1.8 |
| | | Hz, 1H), 6.96 (d, J = 9.1 Hz, 1H), 5.13 (dd, J = 13.5, 5.1 Hz, |
| | | 1H), 4.53 (d, J = 17.9 Hz, 1H), 4.39 (d, J = 18.0 Hz, 1H), 4.35- |
| | | 4.27 (m, 1H), 4.22-4.06 (m, 6H), 3.93-3.83 (m, 1H), 3.38 |
| | | (br d, J = 4.6 Hz, 1H), 3.21-3.09 (m, 3H), 2.96-2.87 (m, 1H), |
| | | 2.71 (s, 2H), 2.66-2.58 (m, 2H), 2.55 (DMSO), 2.46-2.25 |
| | | (m, 7H), 2.10-1.98 (m, 1H), 1.60 (td, J = 10.4, 1.7 Hz, 2H), |
| | | 1.38 (t, J = 6.9 Hz, 3H) |
| 64 | 926.3 | ¹H NMR (500 MHz, DMSO-d₆) δ 8.63 (d, J = 2.1 Hz, 1H), |
| | [M + H]⁺/ | 8.57 (s, 1H), 8.32 (d, J = 2.4 Hz, 1H), 8.09 (s, 1H), 7.87 (s, |
| | 1.72 min | 1H), 7.75 (dd, J = 8.8, 2.5 Hz, 1H), 7.70 (d, J = 8.3 Hz, 1H), |
| | (A); | 7.37-7.29 (m, 4H), 7.26 (d, J = 1.8 Hz, 1H), 6.97 (d, J = 9.0 |
| | | Hz, 1H), 4.32 (br s, 1H), 4.21-4.03 (m, 9H), 3.97-3.84 (m, |
| | | 3H), 3.21-3.09 (m, 3H), 3.00 (s, 1H), 2.78 (t, J = 6.6 Hz, 2H), |
| | | 2.72 (s, 2H), 2.57 (br d, J = 2.0 Hz, 2H), 2.55 (DMSO), 2.45- |
| | | 2.25 (m, 6H), 1.65-1.53 (m, 2H), 1.38 (t, J = 6.9 Hz, 3H) |
| 65 | 956.2 | ¹H NMR (500 MHz, DMSO-d₆) δ 8.63 (d, J = 1.9 Hz, 1H), |
| | [M + H]⁺/ | 8.56 (s, 1H), 8.32 (d, J = 2.3 Hz, 1H), 8.09 (s, 1H), 7.75 (dd, |
| | 1.93 min | J = 8.8, 2.5 Hz, 1H), 7.64 (d, J = 8.4 Hz, 1H), 7.37-7.30 (m, |
| | (A); | 3H), 7.26 (d, J = 2.0 Hz, 1H), 7.14 (s, 1H), 7.06 (br d, J = 8.3 |
| | | Hz, 1H), 6.97 (d, J = 8.9 Hz, 1H), 5.07 (dd, J = 13.3, 5.1 Hz, |
| | | 1H), 4.72 (s, 2H), 4.42-4.37 (m, 1H), 4.30-4.13 (m, 6H), |
| | | 4.07 (br dd, J = 8.2, 4.0 Hz, 1H), 3.94-3.89 (m, 1H), 3.72 (br |
| | | dd, J = 9.7, 4.2 Hz, 1H), 3.42-3.35 (m, 1H), 3.19-3.09 (m, |
| | | 2H), 2.94-2.86 (m, 1H), 2.75-2.68 (m, 2H), 2.65-2.57 (m, |
| | | 2H), 2.55 (DMSO), 2.43-2.28 (m, 6H), 2.25 (br d, J = 4.8 Hz, |
| | | 1H), 2.02-1.97 (m, 1H), 1.64-1.56 (m, 2H), 1.38 (t, J = 6.9 |
| | | Hz, 3H) |
| 66 | 945.4 | ¹H NMR (500 MHz, DMSO-d₆) δ 10.98 (s, 1H), 8.59 (d, |
| | [M + H]⁺/ | J = 1.8 Hz, 1H), 8.53 (s, 1H), 8.29 (d, J = 2.1 Hz, 1H), 7.76- |
| | 1.45 min | 7.70 (m, 2H), 7.50 (s, 1H), 7.42 (br d, J = 7.9 Hz, 1H), 7.32- |
| | (B); | 7.27 (m, 4H), 7.25-7.21 (m, 2H), 7.17 (s, 1H), 7.11-7.06 |
| | | (m, 3H), 6.96 (d, J = 9.0 Hz, 1H), 5.07 (dd, J = 13.2, 5.2 Hz, |
| | | 1H), 4.45-4.39 (m, 2H), 4.34 (s, 1H), 4.14 (q, J = 6.5 Hz, 4H), |
| | | 3.92-3.88 (m, 1H), 3.61 (br dd, J = 9.9, 5.1 Hz, 3H), 3.35 (br |
| | | s, 1H), 3.15-3.08 (m, 2H), 3.04-2.96 (m, 6H), 2.93-2.83 |
| | | (m, 3H), 2.67-2.57 (m, 2H), 2.55 (DMSO), 2.49-2.45 (m, |
| | | 1H), 2.39 (br dd, J = 12.9, 4.2 Hz, 1H), 2.20-2.10 (m, 2H), |
| | | 2.07-1.90 (m, 5H), 1.77-1.69 (m, 2H), 1.64-1.60 (m, 1H), |
| | | 1.58-1.46 (m, 3H), 1.37 (t, J = 7.0 Hz, 3H) |

### Example 67

### N-(1-(5-(3-cyano-6-ethoxypyrazolo[1,5-a]pyridin-4-yl)pyridin-2-yl)-4-((4-((4-(2-(2,6-dioxopiperidin-3-yl)-3-oxoisoindolin-5-yl)piperazin-1-yl)methyl)piperidin-1-yl)methyl)piperidin-4-yl)-2,5-difluorobenzamide

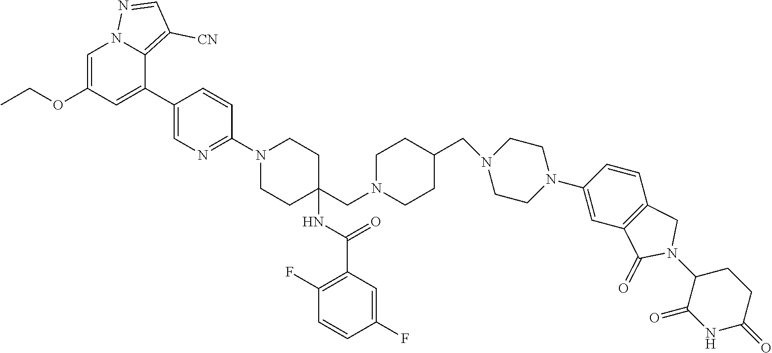 [SMILES: CCOc1cc(-c2ccc(N3CCC(CN4CCC(CN5CCN(c6ccc7c(c6)C(=O)N(C6CCC(=O)NC6=O)C7)CC5)CC4)(NC(=O)c4cc(F)ccc4F)CC3)nc2)c2c(C#N)cnn2c1]

### A. Tert-butyl 4-((4-(2-(2,6-dioxopiperidin-3-yl)-3-oxoisoindolin-5-yl)piperazin-1-yl)methyl)piperidine-1-carboxylate

To the mixture solution of 3-(1-oxo-6-piperazin-1-yl-isoindolin-2-yl)piperidine-2,6-dione (60 mg, 0.18 mmol) and tert-butyl 4-formylpiperidine-1-carboxylate (60 mg, 0.27 mmol) in DCM (5 mL) was added DIEA (50 mg, 0.37 mmol), the mixture solution was stirred at rt for 20 minutes, and then AcOH (25 mg, 0.37 mmol) was added and the mixture solution was stirred at rt for 0.5 h, then the reaction mixture was cooled down to 0 °C, NaBH(OAc)₃ (80 mg, 0.37 mmol) was added and the mixture was stirred at rt overnight. The reaction was concentrated and purified by flash to afford the desired product (80 mg, 83.3% yield) as an off-white solid. MS: m/z [M+H]⁺ 526.

### B. 3-(1-oxo-6-(4-(piperidin-4-ylmethyl)piperazin-1-yl) isoindolin-2-yl)piperidine-2,6-dione

To the mixture solution of tert-butyl 4-[[4-[2-(2,6-dioxo-3-piperidyl)-3-oxo-isoindolin-5-yl]piperazin-1-yl]methyl]piperidine-1-carboxylate (80 mg, 0.1500 mmol) in DCM (10 mL) was added TFA (3 mL), the mixture solution was stirred at rt for 4 h. The reaction was concentrated to afford the desired product (70 mg, quant.), which was used in next step without further purification. MS: m/z [M+H]⁺ 426.

### C. N-(1-(5-(3-cyano-6-ethoxypyrazolo[1,5-a]pyridin-4-yl)pyridin-2-yl)-4-((4-((4-(2-(2,6-dioxopiperidin-3-yl)-3-oxoisoindolin-5-yl)piperazin-1-yl)methyl)piperidin-1-yl)methyl)piperidin-4-yl)-2,5-difluorobenzamide

To the mixture solution of 3-[1-oxo-6-[4-(4-piperidylmethyl)piperazin-1-yl]isoindolin-2-yl]piperidine-2,6-dione (40 mg, 0.09 mmol) and N-[1-[5-(3-cyano-6-ethoxy-pyrazolo[1,5-a]pyridin-4-yl)-2-pyridyl]-4-formyl-4-piperidyl]-2,5-difluoro-benzamide (50 mg, 0.0900 mmol) in DCM (5 mL) was added DIEA (0.01 mL, 0.19 mmol), and the mixture solution was stirred at rt for 2 h, then TIPT (55 mg, 0.1900 mmol) was added and the mixture solution was stirred at rt for 3 days. After cooling the mixture to 0 °C, NaBH(OAc)₃ (40 mg, 0.19 mmol) was added, and the mixture was stirred at rt overnight. The reaction was purified by Prep-HPLC to afford the titled compound (6.9 mg, 7.8% yield) as an off-white solid. ¹H NMR (400 MHz, DMSO-d6) δ 8.63 (s, 1H), 8.57-8.54 (m, 1H), 8.35 (s, 1H), 7.86-7.80 (m, 1H), 7.55-7.46 (m, 2H), 7.45-7.32 (m, 3H), 7.32-7.25 (m, 2H), 7.10-7.04 (m, 1H), 5.12-5.03 (m, 1H), 4.42-4.33 (m, 1H), 4.32-4.19 (m, 3H), 4.18-4.09 (m, 2H), 3.98-3.84 (m, 2H), 3.76-3.69 (m, 5H), 3.31-3.06 (m, 11H), 2.95-2.84 (m, 1H), 2.66-2.57 (m, 1H), 2.46-2.36 (m, 3H), 2.21-2.12 (m, 1H), 2.05-1.92 (m, 3H), 1.77-1.69 (m, 2H), 1.63-1.52 (m, 2H), 1.38 (t, J=7.1 Hz, 3H). MS: m/z [M+H]⁺ 940.4, purity: 99.0%. Prep-HPLC purification condition: Column: SunFire Prep C18 OBD Column, 19×150 mm, 5 μm; Mobile Phase A: Water (0.05% TFA), Mobile Phase B: ACN; Flow rate: 20 mL/min; Gradient: 35% B to 60% B in 5.5 min, 60% B; RT=5.54 min.

### Example 68

### N-(1-(5-(3-cyano-6-ethoxypyrazolo[1,5-a]pyridin-4-yl)pyridin-2-yl)-4-((4-(4-(2-(2,6-dioxopiperidin-3-yl)-3-oxoisoindolin-5-yl)piperazin-1-yl)piperidin-1-yl)methyl)piperidin-4-yl)-2,5-difluorobenzamide

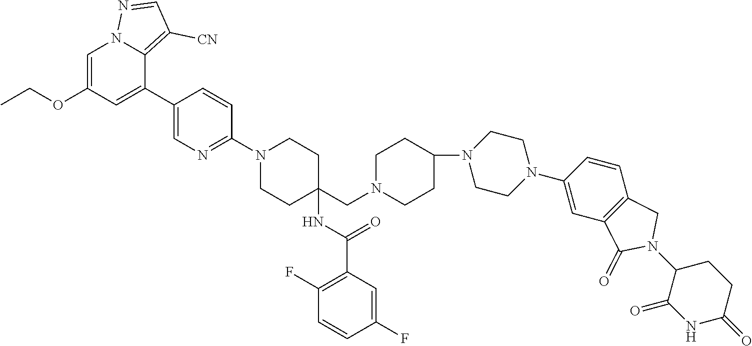 [SMILES: CCOc1cc(-c2ccc(N3CCC(CN4CCC(N5CCN(c6ccc7c(c6)C(=O)N(C6CCC(=O)NC6=O)C7)CC5)CC4)(NC(=O)c4cc(F)ccc4F)CC3)nc2)c2c(C#N)cnn2c1]

### A. N-(4-((1,4-dioxa-8-azaspiro[4.5]decan-8-yl)methyl)-1-(5-(3-cyano-6-ethoxypyrazolo[1,5-a]pyridin-4-yl)pyridin-2-yl)piperidin-4-yl)-2,5-difluorobenzamide

To a stirred solution of N-[1-[5-(3-cyano-6-ethoxy-pyrazolo[1,5-a]pyridin-4-yl)-2-pyridyl]-4-formyl-4-piperidyl]-2,5-difluoro-benzamide (100 mg, 0.19 mmol) in DCM (5 mL) were added 1,4-dioxa-8-azaspiro[4.5]decane (55 mg, 0.3800 mmol) and Titanium (IV) isopropoxide (145 mg, 0.38 mmol) at rt, and the resulting mixture was stirred for 1 h. NaBH(OAc)₃ (80 mg, 0.38 mmol) was added at 0 °C and the mixture was stirred at rt for 2 h. The crude was purified by prep-TLC to afford the desired product (80 mg, 64.5% yield) as a colorless oil. MS: m/z [M+H]⁺ 658.

### B. N-(1-(5-(3-cyano-6-ethoxypyrazolo[1,5-a]pyridin-4-yl)pyridin-2-yl)-4-((4-oxopiperidin-1-yl)methyl)piperidin-4-yl)-2,5-difluorobenzamide

The mixture solution of N-[1-[5-(3-cyano-6-ethoxy-pyrazolo[1,5-a]pyridin-4-yl)-2-pyridyl]-4-(1,4-dioxa-8-azaspiro[4.5]decan-8-ylmethyl)-4-piperidyl]-2,5-difluoro-benzamide (35 mg, 0.05 mmol) in FA (10 mL, 0.05 mmol) was stirred at 80 °C overnight. The reaction was concentrated to afford the desired product (30 mg, 91.9% yield) as a light brown solid, which was used in next step without further purification. MS: m/z [M+H]⁺ 614.

### C. N-(1-(5-(3-cyano-6-ethoxypyrazolo[1,5-a]pyridin-4-yl)pyridin-2-yl)-4-((4-(4-(2-(2,6-dioxopiperidin-3-yl)-3-oxoisoindolin-5-yl)piperazin-1-yl)piperidin-1-yl)methyl)piperidin-4-yl)-2,5-difluorobenzamide

To the mixture solution of N-[1-[5-(3-cyano-6-ethoxy-pyrazolo[1,5-a]pyridin-4-yl)-2-pyridyl]-4-[(4-oxo-1-piperidyl)methyl]-4-piperidyl]-2,5-difluoro-benzamide (30 mg, 0.0500 mmol) and 3-(1-oxo-6-piperazin-1-yl-isoindolin-2-yl)piperidine-2,6-dione (16 mg, 0.0500 mmol) in DCM (4 mL) was added DIEA (0.01 mL, 0.10 mmol), the mixture solution was stirred at rt for 2 h, then TIPT (30 mg, 0.10 mmol) was added, the mixture solution was stirred at rt for 3 days, then the mixture was cooled down to 0 °C, NaBH(OAc)₃ (20 mg, 0.10 mmol) was added, the mixture solution was stirred at rt overnight. The reaction was purified by flash to afford 27 mg crude, then the mixture was purified by Prep-HPLC to afford the titled compound (6.5 mg, 14.2% yield). 1H NMR (400 MHz, DMSO-d6) δ 8.63-8.60 (m, 1H), 8.56-8.53 (m, 1H), 8.36-8.33 (m, 1H), 7.87-7.80 (m, 1H), 7.55-7.47 (m, 2H), 7.45-7.32 (m, 3H), 7.31-7.26 (m, 1H), 7.11-7.05 (m, 1H), 7.08 (d, J=9.1 Hz, 1H), 5.11-5.01 (m, 1H), 4.42-4.34 (m, 1H), 4.31-4.21 (m, 3H), 4.14 (q, J=6.8 Hz, 2H), 3.84-3.72 (m, 13H), 3.28-3.15 (m, 4H), 2.92-2.81 (m, 1H), 2.66-2.57 (m, 1H), 2.46-2.35 (m, 3H), 2.33-2.25 (m, 2H), 2.14-1.97 (m, 3H), 1.80-1.60 (m, 2H), 1.38 (t, J=6.9 Hz, 3H). MS: m/z [M+H]⁺ 926.3, purity: 100%. Prep-HPLC purification condition: Column: SunFire Prep C18 OBD Column, 19×150 mm, 5 μm; Mobile Phase A: Water (0.05% TFA), Mobile Phase B: ACN; Flow rate: 20 mL/min; Gradient: 25% B to 40% B in 5.5 min, 40% B; RT=5.52 min.

### Example 69

### N-(1-(5-(3-cyano-6-ethoxypyrazolo[1,5-a]pyridin-4-yl)pyridin-2-yl)-4-((4-((4-(2-(2,6-dioxopiperidin-3-yl)-1-oxoisoindolin-5-yl)piperazin-1-yl)methyl)piperidin-1-yl)methyl)piperidin-4-yl)-2,5-difluorobenzamide

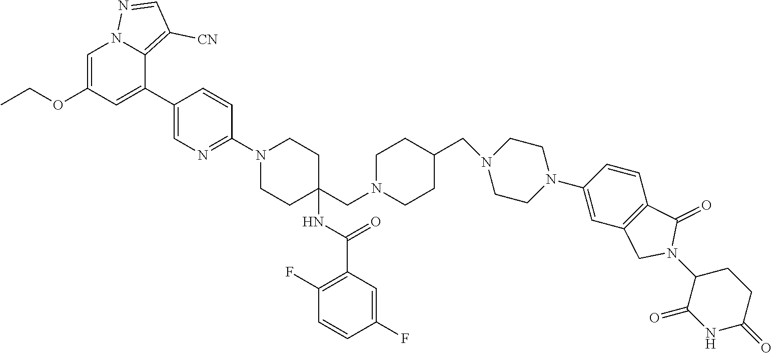 [SMILES: CCOc1cc(-c2ccc(N3CCC(CN4CCC(CN5CCN(c6ccc7c(c6)CN(C6CCC(=O)NC6=O)C7=O)CC5)CC4)(NC(=O)c4cc(F)ccc4F)CC3)nc2)c2c(C#N)cnn2c1]

The title compound was prepared in 9.4% overall yield as a white solid according to the preparation of Example 67 using 3-(1-oxo-5-(piperazin-1-yl) isoindolin-2-yl)piperidine-2,6-dione in STEP A. ¹H NMR (400 MHz, DMSO-d6) δ 8.59 (d, J=2.1 Hz, 1H), 8.53 (s, 1H), 8.33 (d, J=2.5 Hz, 1H), 7.81 (dd, J=8.9, 2.5 Hz, 1H), 7.62 (d, J=8.9 Hz, 1H), 7.49-7.34 (m, 3H), 7.25 (d, J=2.1 Hz, 1H), 7.17-7.14 (m, 2H), 7.04 (d, J=8.9 Hz, 1H), 5.01 (dd, J=13.3, 5.1 Hz, 1H), 4.40-4.35 (m, 1H), 4.27-4.22 (m, 3H), 4.15-4.10 (m, 2H), 3.98-3.89 (m, 4H), 3.66-3.61 (m, 4H), 3.24-3.07 (m, 10H), 2.92-2.82 (m, 1H), 2.66-2.58 (m, 1H), 2.41-2.35 (m, 3H), 2.25-2.09 (m, 1H), 2.04-1.93 (m, 3H), 1.83-1.69 (m, 2H), 1.61-1.57 (m, 2H), 1.37 (t, J=6.9 Hz, 3H). MS: m/z [M+H]⁺ 940.5. Prep-HPLC purification condition: Column: SunFire Prep C18 OBD Column, 19×150 mm, 5 μm; Mobile Phase A: Water (0.05% TFA), Mobile Phase B: ACN; Flow rate: 20 mL/min; Gradient: 30% B to 55% B in 5.3 min, 55% B; RT=5.63 min.

### Example 70

### N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)azetidine-1-carbonyl)piperidin-1-yl)methyl)piperidin-4-yl)-2,5-difluorobenzamide

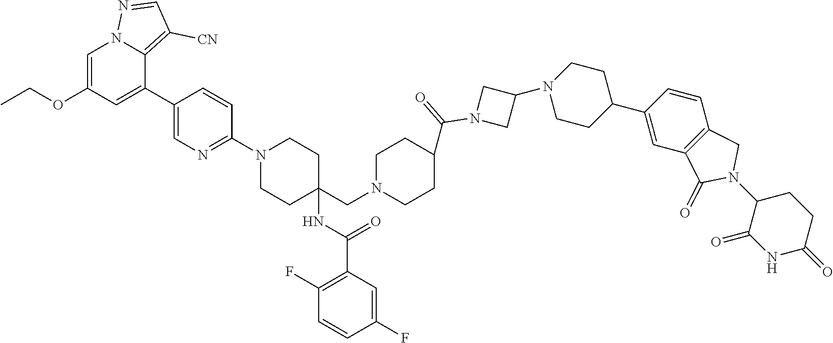 [SMILES: CCOc1cc(-c2ccc(N3CCC(CN4CCC(C(=O)N5CC(N6CCC(c7ccc8c(c7)C(=O)N(C7CCC(=O)NC7=O)C8)CC6)C5)CC4)(NC(=O)c4cc(F)ccc4F)CC3)nc2)c2c(C#N)cnn2c1]

### A. Tert-butyl 3-(4-(2-(2,6-dioxopiperidin-3-yl)-3-oxoisoindolin-5-yl)piperidin-1-yl)azetidine-1-carboxylate

To a solution of 3-[1-oxo-6-(4-piperidyl) isoindolin-2-yl]piperidine-2,6-dione (100 mg, 0.31 mmol) and tert-butyl 3-oxoazetidine-1-carboxylate (104.58 mg, 0.61 mmol) in DCM (10 mL) were added DIEA (78.81 mg, 0.61 mmol), Titanium (IV) isopropoxide (173.5 mg, 0.61 mmol). The mixture was stirred at rt for 1 h. NaBH(OAc)₃ (129.48 mg, 0.61 mmol) was added at 0 °C and the resulting mixture was stirred at rt for another 2 h. It was concentrated, the crude was purified by reversed phase column chromatography to afford the desired product (100 mg, 67.8% yield) as a yellow oil. MS: m/z [M+H]⁺ 483.

### B. 3-(6-(1-(Azetidin-3-yl)piperidin-4-yl)-1-oxoisoindolin-2-yl)piperidine-2,6-dione

To a solution of tert-butyl 3-[4-[2-(2,6-dioxo-3-piperidyl)-3-oxo-isoindolin-5-yl]-1-piperidyl]azetidine-1-carboxylate (300 mg, 0.62 mmol) in DCM (5 mL) was added TFA (2 mL). The mixture was stirred at rt for 1 h. The reaction was concentrated to afford product, which was used directly in the next step without further purification.

### C. Tert-butyl 4-(3-(4-(2-(2,6-dioxopiperidin-3-yl)-3-oxoisoindolin-5-yl)piperidin-1-yl)azetidine-1-carbonyl)piperidine-1-carboxylate

To a solution of 3-[6-[1-(azetidin-3-yl)-4-piperidyl]-1-oxo-isoindolin-2-yl]piperidine-2,6-dione (80 mg, 0.21 mmol), 1-tert-butoxycarbonylpiperidine-4-carboxylic acid (71.94 mg, 0.31 mmol) in DMA (1 mL) were added HATU (119.3 mg, 0.31 mmol), DIEA (48 mg, 0.37 mmol). The mixture was stirred at rt for 2 h. LCMS showed the reaction was complete, the crude was purified by reversed phase column chromatography to afford the desired product (80 mg, 64.4% yield) as a yellow oil. MS: m/z: [M+H]⁺ 594.

### D. 3-(Oxo-6-(1-(1-(piperidine-4-carbonyl)azetidin-3-yl)piperidin-4-yl) isoindolin-2-yl)piperidine-2,6-dione

To a solution of tert-butyl 4-[3-[4-[2-(2,6-dioxo-3-piperidyl)-3-oxo-isoindolin-5-yl]-1-piperidyl]azetidine-1-carbonyl]piperidine-1-carboxylate (95 mg, 0.16 mmol) in 1,4-Dioxane (2 mL) was added HCl (4 M in dioxane) (2 mL). The mixture was stirred at rt for 2 h. It was concentrated to afford crude product, which was used directly in the next step without further purification. MS: m/z [M+H]⁺ 494.

### E. N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)azetidine-1-carbonyl)piperidin-1-yl)methyl)piperidin-4-yl)-2,5-difluorobenzamide

To a solution of N-[1-[5-(3-cyano-6-ethoxy-pyrazolo[1,5-a]pyridin-4-yl)-2-pyridyl]-4-formyl-4-piperidyl]-2,5-difluoro-benzamide (40 mg, 0.08 mmol), 3-[1-oxo-6-[1-[1-(piperidine-4-carbonyl)azetidin-3-yl]-4-piperidyl]isoindolin-2-yl]piperidine-2,6-dione (37.22 mg, 0.08 mmol) in DCM (2 mL) were added Titanium (IV) isopropoxide (42.83 mg, 0.15 mmol), DIEA (19.45 mg, 0.15 mmol). The mixture was stirred at rt for 3 days. NaBH(OAc)₃ (31.97 mg, 0.15 mmol) was added at 0 °C. The mixture was stirred at rt for another 16 h. It was concentrated and purified by prep-HPLC to afford the titled product (8.4 mg, 11.0% yield) as a white solid. ¹H NMR (400 MHz, DMSO-d6) δ 11.00 (s, 1H), 9.07 (s, 1H), 8.68-8.67 (m, 1H), 8.62-8.59 (m, 1H), 8.54 (s, 1H), 8.37 (d, J=2.6 Hz, 1H), 7.82 (dd, J=8.7, 2.6 Hz, 1H), 7.61-7.40 (m, 5H), 7.28-7.26 (m, 1H), 7.06 (d, J=8.7 Hz, 1H), 5.11 (dd, J=13.2, 5.1 Hz, 1H), 4.49-4.43 (m, 3H), 4.36-4.23 (m, 3H), 4.19-4.12 (m, 4H), 3.76-3.65 (m, 8H), 3.26-3.13 (m, 4H), 3.07-2.86 (m, 4H), 2.68-2.59 (m, 2H), 2.47-2.34 (m, 3H), 2.17-2.11 (m, 2H), 2.05-1.83 (m, 6H), 1.79-1.71 (m, 2H), 1.39 (t, J=6.9 Hz, 3H). MS: m/z [M+H]⁺ 1008. Prep-HPLC purification condition: Column: Column: SunFire Prep C18 OBD Column, 19×150 mm, 5 μm; Mobile Phase A: Water (0.05% TFA), Mobile Phase B: ACN; Flow rate: 20 mL/min; Gradient: 35% B to 50% B in 5.3 min, 50% B; RT=5.52 min.

### Example 71

### N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)azetidin-1-yl)piperidin-1-yl)methyl)piperidin-4-yl)-2,5-difluorobenzamide

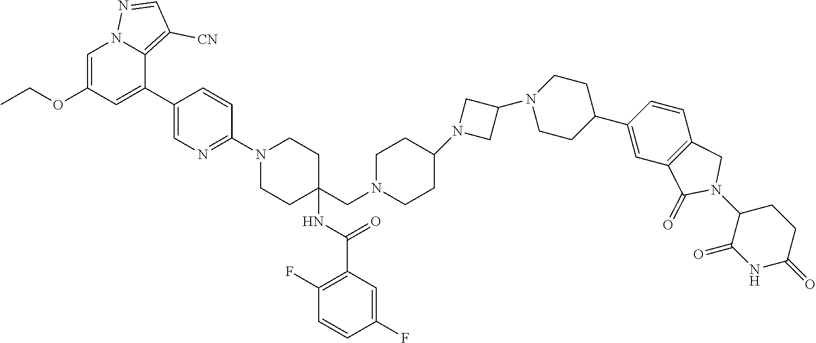 [SMILES: CCOc1cc(-c2ccc(N3CCC(CN4CCC(N5CC(N6CCC(c7ccc8c(c7)C(=O)N(C7CCC(=O)NC7=O)C8)CC6)C5)CC4)(NC(=O)c4cc(F)ccc4F)CC3)nc2)c2c(C#N)cnn2c1]

The title compound was prepared in 5.1% overall yield as a white solid according to the preparation of Example 67 using 3-(1-oxo-5-(piperazin-1-yl) isoindolin-2-yl)piperidine-2,6-dione in STEP A. ¹H NMR (400 MHz, DMSO-d6) δ 11.00 (s, 1H), 8.67 (d, J=2.0 Hz, 1H), 8.59 (s, 2H), 8.36 (d, J=2.4 Hz, 1H), 7.82 (dd, J=8.8, 2.4 Hz, 1H), 7.63-7.52 (m, 3H), 7.49-7.36 (m, 3H), 7.28 (d, J=2.0 Hz, 1H), 7.06 (d, J=8.8 Hz, 1H), 5.11 (dd, J=13.3, 5.1 Hz, 1H), 4.47-4.42 (m, 1H), 4.37-4.23 (m, 4H), 4.19-4.14 (m, 3H), 3.94-3.87 (m, 3H), 3.79-3.69 (m, 7H), 3.35-3.30 (m, 3H), 3.24-3.18 (m, 3H), 2.96-2.87 (m, 2H), 2.79-2.71 (m, 1H), 2.67-2.55 (m, 1H), 2.47-2.33 (m, 3H), 2.11-1.96 (m, 5H), 1.91-1.84 (m, 2H), 1.75-1.67 (m, 3H), 1.39 (t, J=7.0 Hz, 3H). MS: m/z [M+H]⁺ 980. Prep-HPLC purification condition: Column: SunFire Prep C18 OBD Column, 19×150 mm, 5 μm; Mobile Phase A: Water (0.05% TFA), Mobile Phase B: ACN; Flow rate: 20 mL/min; Gradient: 20% B to 30% B in 7 min, 30% B; RT=6.57 min.

### Example 72

### N-(1-(5-(3-cyano-6-ethoxypyrazolo[1,5-a]pyridin-4-yl)pyridin-2-yl)-4-((4-((3-(4-(2-(2,6-dioxopiperidin-3-yl)-3-oxoisoindolin-5-yl)piperidin-1-yl)azetidin-1-yl)methyl)piperidin-1-yl)methyl)piperidin-4-yl)-2,5-difluorobenzamide

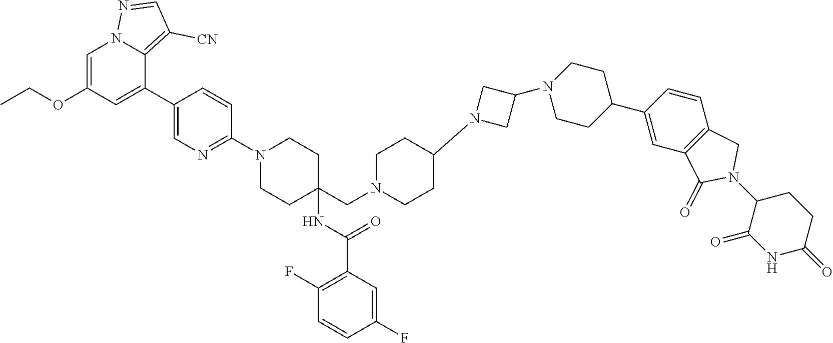 [SMILES: CCOc1cc(-c2ccc(N3CCC(CN4CCC(N5CC(N6CCC(c7ccc8c(c7)C(=O)N(C7CCC(=O)NC7=O)C8)CC6)C5)CC4)(NC(=O)c4cc(F)ccc4F)CC3)nc2)c2c(C#N)cnn2c1]

The title compound was prepared in 3.65% overall yield as a white solid according to the preparation of Example 70 using tert-butyl 4-formylpiperidine-1-carboxylate in STEP A. ¹H NMR (400 MHz, DMSO-d6) δ 11.00 (s, 1H), 8.67 (d, J=2.1 Hz, 1H), 8.59 (s, 2H), 8.37 (d, J=2.5 Hz, 1H), 7.82 (dd, J=8.8, 2.5 Hz, 1H), 7.64-7.52 (m, 3H), 7.51-7.46 (m, 1H), 7.44-7.36 (m, 2H), 7.28 (d, J=2.1 Hz, 1H), 7.06 (d, J=8.8 Hz, 1H), 5.11 (dd, J=13.2, 5.1 Hz, 1H), 4.47-4.42 (m, 2H), 4.34-4.23 (m, 5H), 4.19-4.14 (m, 2H), 34.01-3.88 (m, 1H), 3.77-3.68 (m, 7H), 3.34-3.27 (m, 3H), 3.24-3.20 (m, 2H), 3.17-3.06 (m, 3H), 2.96-2.87 (m, 2H), 2.70-2.57 (m, 2H), 2.45-2.32 (m, 3H), 2.10-1.96 (m, 3H), 1.90-1.84 (m, 4H), 1.77-1.67 (m, 2H), 1.62-1.54 (m, 2H), 1.39 (t, J=6.9 Hz, 3H). MS: m/z [M+H]⁺ 994. Prep-HPLC purification condition: Column: SunFire Prep C18 OBD Column, 19×150 mm, 5 μm; Mobile Phase A: Water (0.05% TFA), Mobile Phase B: ACN; Flow rate: 20 mL/min; Gradient: 20% B to 28% B in 7 min, 28% B; RT=6.75 min.

### Example 73

### N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)propanoyl)piperazin-1-yl)methyl)piperidin-4-yl)-1-fluorocyclopropane-1-carboxamide

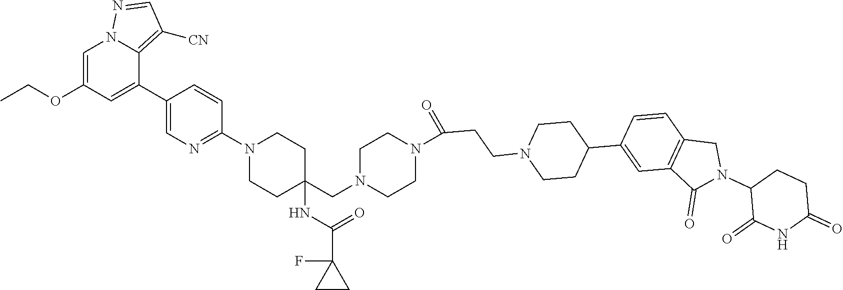 [SMILES: CCOc1cc(-c2ccc(N3CCC(CN4CCN(C(=O)CCN5CCC(c6ccc7c(c6)C(=O)N(C6CCC(=O)NC6=O)C7)CC5)CC4)(NC(=O)C4(F)CC4)CC3)nc2)c2c(C#N)cnn2c1]

The title compound was prepared in 21.5% yield as a white solid according to the preparation of Example 2 using N-[1-[5-(3-cyano-6-ethoxy-pyrazolo[1,5-a]pyridin-4-yl)-2-pyridyl]-4-(piperazin-1-ylmethyl)-4-piperidyl]-1-fluoro-cyclopropanecarboxamide and 3-[4-[2-(2,6-dioxo-3-piperidyl)-3-oxo-isoindolin-5-yl]-1-piperidyl]propanoic acid. ¹H NMR (400 MHz, DMSO-d6) δ 11.01 (s, 1H), 8.66 (s, 1H), 8.59 (s, 1H), 8.33 (s, 1H), 7.80-7.74 (m, 1H), 7.61-7.50 (m, 4H), 7.28 (s, 1H), 7.02-6.93 (m, 1H), 5.17-5.08 (m, 1H), 4.42 (d, J=17.0 Hz, 1H), 4.28 (d, J=17.0 Hz, 1H), 4.21-4.05 (m, 4H), 3.51-3.40 (m, 4H), 3.20-2.85 (m, 6H), 2.73-2.55 (m, 6H), 2.47-2.32 (m, 7H), 2.19-1.96 (m, 3H), 1.87-1.55 (m, 7H), 1.45-1.34 (m, 3H), 1.34-1.22 (m, 2H), 1.22-1.13 (m, 2H). MS: m/z [M+H]⁺ 928.5. Prep-HPLC purification condition: Column: XBridge BEH C18 OBD Prep Column, 19×250 mm, 5 μm; Mobile Phase A: Water (10 mmol/L NH₄HCO₃), Mobile Phase B: ACN; Flow rate: 25 mL/min; Gradient: 47% B to 57% B in 6 min, 57% B; RT=5.62 min.

### Example 74

### N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)cyclobutane-1-carbonyl)piperazin-1-yl)methyl)piperidin-4-yl)-2,5-difluorobenzamide

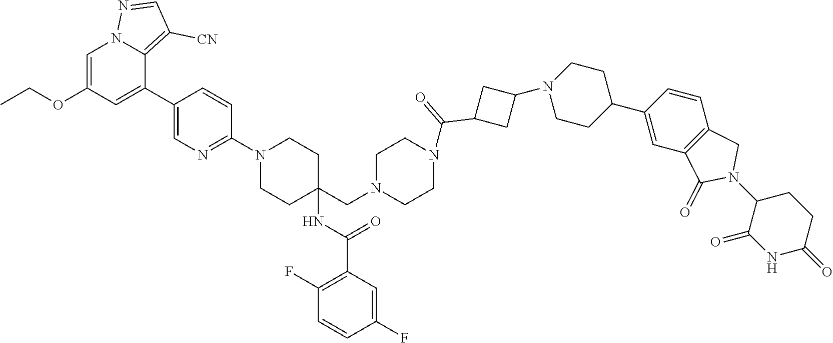 [SMILES: CCOc1cc(-c2ccc(N3CCC(CN4CCN(C(=O)C5CC(N6CCC(c7ccc8c(c7)C(=O)N(C7CCC(=O)NC7=O)C8)CC6)C5)CC4)(NC(=O)c4cc(F)ccc4F)CC3)nc2)c2c(C#N)cnn2c1]

### A. Tert-butyl 3-(4-(2-(2,6-dioxopiperidin-3-yl)-3-oxoisoindolin-5-yl)piperidin-1-yl)cyclobutane-1-carboxylate

To the mixture solution of 3-[1-oxo-6-(4-piperidyl) isoindolin-2-yl]piperidine-2,6-dione (60 mg, 0.18 mmol) and tert-butyl 3-oxocyclobutanecarboxylate (50 mg, 0.27 mmol) in DCM (5 mL) was added DIEA (0.03 mL, 0.37 mmol), the mixture solution was stirred at rt for 5 minutes, and then TIPT (105 mg, 0.37 mmol) was added, and the mixture solution was stirred at rt for 0.5 h. After cooling to 0 °C, NaBH(OAc)₃ (80 mg, 0.37 mmol) was added, and the mixture solution was stirred at rt for 5 h, the reaction was purified by Prep-HPLC to afford the desired product (60 mg, 68% yield) as an off-white solid. MS: m/z [M+H]⁺ 482.

### B. 3-(4-(2-(2,6-Dioxopiperidin-3-yl)-3-oxoisoindolin-5-yl)piperidin-1-yl)cyclobutane-1-carboxylic acid

To the mixture solution of tert-butyl 3-[4-[2-(2,6-dioxo-3-piperidyl)-3-oxo-isoindolin-5-yl]-1-piperidyl]cyclobutanecarboxylate (55 mg, 0.11 mmol) in DCM (10 mL) was added TFA (3 mL), the mixture solution was stirred at rt for 4 h. The reaction was concentrated to afford the desired product (45 mg, 92.6% yield), which was used in next step without further purification. MS: m/z [M+H]⁺ 426.

### C. N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)cyclobutane-1-carbonyl)piperazin-1-yl)methyl)piperidin-4-yl)-2,5-difluorobenzamide

To the mixture solution of 3-[4-[2-(2,6-dioxo-3-piperidyl)-3-oxo-isoindolin-5-yl]-1-piperidyl]cyclobutanecarboxylic acid (35 mg, 0.08 mmol), N-[1-[5-(3-cyano-6-ethoxy-pyrazolo[1,5-a]pyridin-4-yl)-2-pyridyl]-4-(piperazin-1-ylmethyl)-4-piperidyl]-2,5-difluoro-benzamide (50 mg, 0.08 mmol) and DIEA (0.02 mL, 0.25 mmol) in DMF, was added HATU (50 mg, 0.12 mmol) at −5 °C, the mixture solution was stirred at 0 °C for 2 h. The reaction was purified by Prep-HPLC to afford the titled compound (28.1 mg, 33.4% yield) as a white solid. ¹H NMR (300 MHz, DMSO-d6) δ 10.98 (s, 1H), 8.65 (d, J=2.1 Hz, 1H), 8.58 (s, 1H), 8.33 (d, J=2.5 Hz, 1H), 8.14 (s, 1H), 7.76 (dd, J=8.9, 2.6 Hz, 1H), 7.59-7.50 (m, 3H), 7.41-7.31 (m, 3H), 7.27 (d, J=2.1 Hz, 1H), 7.01-6.94 (m, 1H), 5.11 (dd, J=13.2, 5.1 Hz, 1H), 4.41 (d, J=17.1 Hz, 1H), 4.34-4.10 (m, 5H), 3.47-3.39 (m, 2H), 3.31-3.27 (m, 1H), 3.24-3.07 (m, 2H), 3.03-2.83 (m, 4H), 2.78-2.68 (m, 2H), 2.67-2.53 (m, 4H), 2.47-2.39 (m, 4H), 2.40-2.29 (m, 3H), 2.25-2.14 (m, 2H), 2.05-1.89 (m, 3H), 1.86-1.73 (m, 4H), 1.70-1.52 (m, 4H), 1.38 (t, J=6.9 Hz, 3H). MS: m/z [M+H]⁺ 1009.4. Prep-HPLC purification condition: Column: XBridge Prep OBD C18 Column, 30×150 mm, 5 μm; Mobile Phase A: Water (10 mmol/L NH₄HCO_{3+0.1}% NH₃·H₂O), Mobile Phase B: ACN; Flow rate: 60 mL/min; Gradient: 47% B to 57% B in 12 min, 57% B; RT=10.22 min.

### Example 75

### N-(1-(5-(3-cyano-6-ethoxypyrazolo[1,5-a]pyridin-4-yl)pyridin-2-yl)-4-(4-(1-(1-(2-(2,6-dioxopiperidin-3-yl)-3-oxoisoindolin-5-yl)piperidin-4-yl)azetidin-3-yl)piperazin-1-yl)methyl)piperidin-4-yl)-2,5-difluorobenzamide

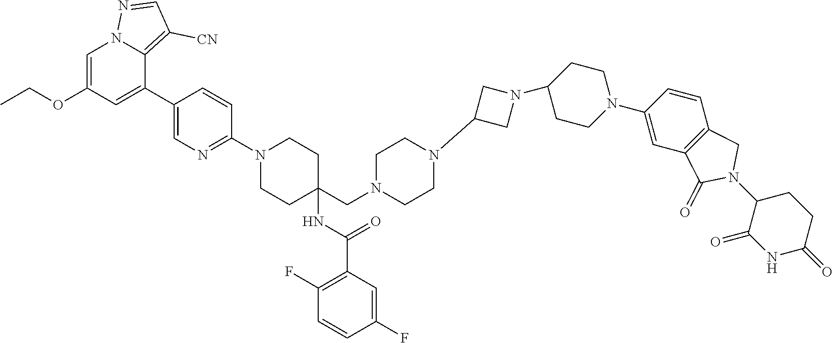 [SMILES: CCOc1cc(-c2ccc(N3CCC(CN4CCN(C5CN(C6CCN(c7ccc8c(c7)C(=O)N(C7CCC(=O)NC7=O)C8)CC6)C5)CC4)(NC(=O)c4cc(F)ccc4F)CC3)nc2)c2c(C#N)cnn2c1]

### A. 3-[6-(1,4-Dioxa-8-azaspiro[4.5]decan-8-yl)-1-oxo-isoindolin-2-yl]piperidine-2,6-dione

To a 5 microwave vial, 3-(6-bromo-1-oxo-isoindolin-2-yl)piperidine-2,6-dione (500 mg, 1.55 mmol), 1,4-dioxa-8-azaspiro[4.5]decane (443 mg, 3.09 mmol), (DiMelHeptCl)Pd(cinnamyl) C1 (130 mg, 0.12 mmol), Cs₂CO₃ (2012 mg, 6.19 mmol) were suspended in 1,4-dioxane (18 mL). The reaction was placed under vacuum, sonicated and backfilled with nitrogen. The mixture was stirred at rt for 16 h. It was concentrated and purified by column chromatography to afford the desired product (430 mg, 72.1% yield) as a white solid. MS: m/z [M+H]⁺ 386.

### B. 3-(Oxo-6-(4-oxopiperidin-1-yl) isoindolin-2-yl)piperidine-2,6-dione

A mixture solution of 3-[6-(1,4-dioxa-8-azaspiro[4.5]decan-8-yl)-1-oxo-isoindolin-2-yl]piperidine-2,6-dione (200 mg, 0.52 mmol) in formic acid (8 mL) was heated at 60 °C for 20 h. It was concentrated to afford crude product which was used in the next step without further purification. MS: m/z: [M+H]⁺ 342.

### C. tert-butyl 3-(4-((1-(5-(3-cyano-6-ethoxypyrazolo[1,5-a]pyridin-4-yl)pyridin-2-yl)-4-(2,5-difluorobenzamido)piperidin-4-yl)methyl)piperazin-1-yl)azetidine-1-carboxylate

To a solution of N-[1-[5-(3-cyano-6-ethoxy-pyrazolo[1,5-a]pyridin-4-yl)-2-pyridyl]-4-formyl-4-piperidyl]-2,5-difluoro-benzamide (80 mg, 0.15 mmol), tert-butyl 3-piperazin-1-ylazetidine-1-carboxylate (54.59 mg, 0.23 mmol) in DCM (5 mL) was added TIPT (85.65 mg, 0.30 mmol). The mixture was stirred at rt for 16 h. NaBH(OAc)₃ (63.94 mg, 0.30 mmol) was added at 0 °C. The mixture was stirred at rt for another 4 h. It was purified by Prep-TLC (EA as eluent) to afford the desired product (70 mg, 61.4% yield) as a light-yellow solid. MS: m/z [M+H]⁺ 756.

### D. N-(4-((4-(azetidin-3-yl)piperazin-1-yl)methyl)-1-(5-(3-cyano-6-ethoxypyrazolo[1,5-a]pyridin-4-yl)pyridin-2-yl)piperidin-4-yl)-2,5-difluorobenzamide

To a solution of tert-butyl 3-[4-[[1-[5-(3-cyano-6-ethoxy-pyrazolo[1,5-a]pyridin-4-yl)-2-pyridyl]-4-[(2,5-difluorobenzoyl)amino]-4-piperidyl]methyl]piperazin-1-yl]azetidine-1-carboxylate (70 mg, 0.09 mmol) in 1,4-dioxane (2 mL) was added HCl (4 M in dioxane) (2 mL). The mixture was stirred at rt for 2 h. It was concentrated and used directly in the next step without further purification. MS: m/z [M+H]⁺ 656.

### E. N-(1-(5-(3-cyano-6-ethoxypyrazolo[1,5-a]pyridin-4-yl)pyridin-2-yl)-4-((4-(1-(1-(2-(2,6-dioxopiperidin-3-yl)-3-oxoisoindolin-5-yl)piperidin-4-yl)azetidin-3-yl)piperazin-1-yl)methyl)piperidin-4-yl)-2,5-difluorobenzamide

To a solution of N-[4-[[4-(azetidin-3-yl)piperazin-1-yl]methyl]-1-[5-(3-cyano-6-ethoxy-pyrazolo[1,5-a]pyridin-4-yl)-2-pyridyl]-4-piperidyl]-2,5-difluoro-benzamide (40 mg, 0.06 mmol), 3-[1-oxo-6-(4-oxo-1-piperidyl) isoindolin-2-yl]piperidine-2,6-dione (20.82 mg, 0.06 mmol) in DCM (3 mL) were added TIPT (34.65 mg, 0.12 mmol), DIEA (15.74 mg, 0.12 mmol). The mixture was stirred at rt for 3 days, then NaBH(OAc)₃ (25.86 mg, 0.12 mmol) was added at 0 °C. The mixture was stirred at rt for another 16 h. It was concentrated, the crude was purified by Prep-HPLC to afford the titled product (1.2 mg, 1.9% yield) as a white solid. ¹H NMR (400 MHz, DMSO-d6) δ 10.99 (s, 1H), 10.12-9.50 (m, 1H), 8.68 (d, J=2.5 Hz, 1H), 8.59 (s, 1H), 8.36 (d, J=2.5 Hz, 1H), 7.84-7.77 (m, 1H), 7.47-7.40 (m, 4H), 7.29-7.21 (m, 3H), 7.06-7.03 (m, 1H), 5.10 (dd, J=13.3, 5.1 Hz, 1H), 4.39-3.99 (m, 10H), 3.93-3.88 (m, 2H), 3.67-3.51 (m, 5H), 3.26-3.09 (m, 3H), 3.06-2.81 (m, 3H), 2.79-2.70 (m, 3H), 2.64-2.55 (m, 2H), 2.43-2.29 (m, 4H), 2.04-1.90 (m, 3H), 1.74-1.66 (m, 2H), 1.51-1.33 (m, 6H). MS: m/z [M+H]⁺ 981. Prep-HPLC purification condition: Column: Welch Utimate HS—C18, 21.2×250 mm, 7 μm; Mobile Phase A: Water (0.05% TFA), Mobile Phase B: ACN (0.1% TFA); Flow rate: 30 mL/min; Gradient: 25% B to 35% B in 8 min, 35% B; RT=8.05 min.

### Example 76

### 1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)propanoyl)piperazin-1-yl)methyl)-N-isopropylpiperidine-4-carboxamide

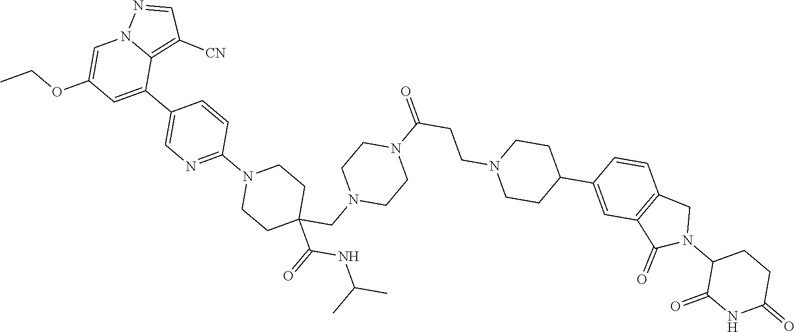 [SMILES: CCOc1cc(-c2ccc(N3CCC(CN4CCN(C(=O)CCN5CCC(c6ccc7c(c6)C(=O)N(C6CCC(=O)NC6=O)C7)CC5)CC4)(C(=O)NC(C)C)CC3)nc2)c2c(C#N)cnn2c1]

To the mixture solution of 3-[4-[2-(2,6-dioxo-3-piperidyl)-3-oxo-isoindolin-5-yl]-1-piperidyl]propanoic acid (20 mg, 0.05 mmol) and 1-[5-(3-cyano-6-ethoxy-pyrazolo[1,5-a]pyridin-4-yl)-2-pyridyl]-N-isopropyl-4-(piperazin-1-ylmethyl)piperidine-4-carboxamide (27 mg, 0.05 mmol) in DMF (3 mL) was added T₃P (46 mg, 0.08 mmol) and DIEA (0.01 mL, 0.15 mmol), and stirred at −5 °C for 1 h, the reaction was purified by Prep-HPLC to afford the titled compound (16.9 mg, 36.6% yield) as a white solid. ¹H NMR (400 MHz, DMSO-d6) δ 11.00 (s, 1H), 8.69-8.63 (m, 1H), 8.59-8.55 (m, 1H), 8.35-8.31 (m, 1H), 7.88-7.76 (m, 2H), 7.64-7.51 (m, 3H), 7.29-7.25 (m, 1H), 7.05-7.00 (m, 1H), 5.15-5.05 (m, 1H), 4.44 (d, J=17.0 Hz, 1H), 4.31 (d, J=17.0 Hz, 1H), 4.16 (q, J=7.0 Hz, 2H), 4.05-3.90 (m, 3H), 3.78-3.61 (m, 4H), 3.39-3.30 (m, 5H), 3.17-3.06 (m, 2H), 3.04-2.83 (s, 11H), 2.67-2.57 (m, 1H), 2.45-2.35 (m, 1H), 2.26-2.15 (m, 2H), 2.12-1.85 (m, 5H), 1.70-1.56 (m, 2H), 1.39 (t, J=6.8 Hz, 3H), 1.12 (d, J=6.5 Hz, 6H). Analytical LCMS ESI-MS(+) m/z 912.6 [M+H]⁺, RT=1.13 min, purity: 95% (Method Q). Prep-HPLC purification condition: Column: SunFire Prep C18 OBD Column, 19×150 mm, 5 μm; Mobile Phase A: Water (0.05% TFA), Mobile Phase B: ACN; Flow rate: 25 mL/min; Gradient: 20% B to 60% B in 6 min, 60% B; RT=5.78 min.

### Example 77

### 1-(5-(3-Cyano-6-ethoxypyrazolo[1,5-a]pyridin-4-yl)pyridin-2-yl)-4-((4-(3-(4-(2-(2,6-dioxopiperidin-3-yl)-1-oxoisoindolin-5-yl)piperazin-1-yl)propanoyl)piperazin-1-yl)methyl)-N-isopropylpiperidine-4-carboxamide

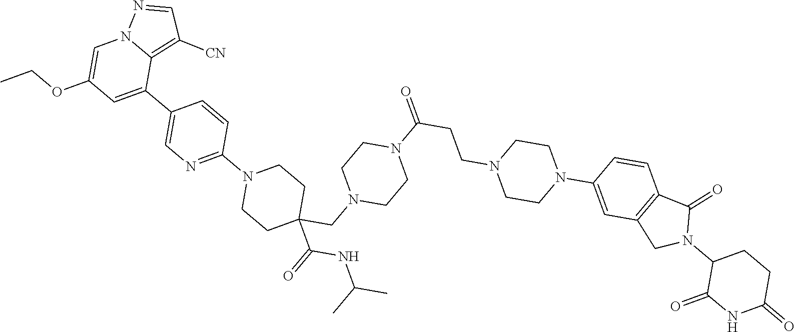 [SMILES: CCOc1cc(-c2ccc(N3CCC(CN4CCN(C(=O)CCN5CCN(c6ccc7c(c6)CN(C6CCC(=O)NC6=O)C7=O)CC5)CC4)(C(=O)NC(C)C)CC3)nc2)c2c(C#N)cnn2c1]

The title compound was prepared in 17.4% overall yield as a white solid according to the preparation of Example 76 using 3-(1-oxo-5-(piperazin-1-yl) isoindolin-2-yl)piperidine-2,6-dione in STEP A. ¹H NMR (400 MHz, DMSO-d6) δ 10.97 (s, 1H), 9.77 (s, 1H), 8.67 (d, J=2.1 Hz, 1H), 8.58 (s, 1H), 8.33 (d, J=2.6 Hz, 1H), 7.79 (dd, J=9.0, 2.6 Hz, 1H), 7.60 (d, J=8.4 Hz, 1H), 7.28 (d, J=2.1 Hz, 1H), 7.22-7.13 (m, 2H), 6.99 (d, J=9.0 Hz, 1H), 5.07 (dd, J=13.3, 5.1 Hz, 1H), 4.37 (d, J=17.2 Hz, 1H), 4.24 (d, J=17.0 Hz, 1H), 4.16 (q, J=7.0 Hz, 2H), 4.09-3.93 (m, 5H), 3.72-3.59 (m, 4H), 3.44-3.34 (m, 8H), 3.34-3.11 (m, 8H), 2.98-2.86 (m, 3H), 2.64-2.56 (m, 1H), 2.47-2.35 (m, 1H), 2.24-2.13 (m, 2H), 2.03-1.94 (m, 1H), 1.58 (s, 2H), 1.39 (t, J=6.9 Hz, 3H), 1.11 (d, J=6.6 Hz, 6H). MS: m/z [M+H]⁺ 913.6, purity: 99.0%. Prep-HPLC purification condition: Column: SunFire C18 OBD Prep Column, 19×250 mm, 5 μm; Mobile Phase A: Water (0.05% TFA), Mobile Phase B: ACN; Flow rate: 25 mL/min; Gradient: 20% B to 23% B in 8 min, 23% B; RT=6.87 min.

### Example 78

### 1-(5-{3-cyano-6-ethoxypyrazolo[1,5-a]pyridin-4-yl}pyridin-2-yl)-4-{[4-(3-{4-[2-(2,6-dioxopiperidin-3-yl)-3-oxo-2,3-dihydro-1H-isoindol-5-yl]piperidin-1-yl}propanoyl)piperazin-1-yl]methyl}-N-(2-methylpropyl)piperidine-4-carboxamide

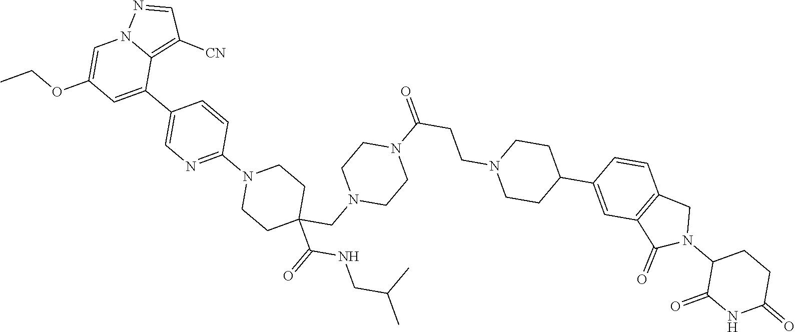 [SMILES: CCOc1cc(-c2ccc(N3CCC(CN4CCN(C(=O)CCN5CCC(c6ccc7c(c6)C(=O)N(C6CCC(=O)NC6=O)C7)CC5)CC4)(C(=O)NCC(C)C)CC3)nc2)c2c(C#N)cnn2c1]

To a stirred solution of 3-[4-[2-(2,6-dioxo-3-piperidyl)-3-oxo-isoindolin-5-yl]-1-piperidyl]propanoic acid (40 mg, 0.10 mmol) in DMF (2 mL) were added 1-[5-(3-cyano-6-ethoxy-pyrazolo[1,5-a]pyridin-4-yl)-2-pyridyl]-N-isobutyl-4-(piperaz-1-ylmethyl)piperidine-4-carboxamide (81.82 mg, 0.15 mmol), T₃P (63.69 mg, 0.20 mmol) and DIEA (0.02 mL, 0.3000 mmol) at 0 °C. The reaction mixture was stirred at room temperature for 2 h. LCMS showed the starting material was consumed completely. The mixture was directly purified by reversed phase column to obtain the titled compound (20.1 mg, 21.63% yield) as a white solid. ¹H NMR (400 MHz, DMSO-d6) δ 10.99 (s, 1H), 8.65 (d, J=2.1 Hz, 1H), 8.57 (s, 1H), 8.31 (d, J=2.6 Hz, 1H), 7.85-7.70 (m, 2H), 7.55 (d, J=12.6 Hz, 3H), 7.27 (d, J=2.1 Hz, 1H), 6.93 (d, J=9.0 Hz, 1H), 5.11 (dd, J=13.3, 5.1 Hz, 1H), 4.42 (d, J=17.1 Hz, 1H), 4.29 (d, J=17.1 Hz, 1H), 4.16 (q, J=6.9 Hz, 2H), 4.07 (d, J=12.8 Hz, 2H), 3.16-2.85 (m, 8H), 2.71-2.53 (m, 7H), 2.49-2.33 (m, 7H), 2.25-2.07 (m, 4H), 2.05-1.95 (m, 1H), 1.87-1.65 (m, 5H), 1.57-1.46 (m, 3H), 1.38 (t, J=6.9 Hz, 3H), 0.92 (t, J=7.1 Hz, 1H), 0.86 (d, J=6.6 Hz, 6H). MS: m/z [M+H]⁺ 927. Prep-HPLC purification conditions: Column: SunFire Prep C18 OBD Column, 19×150 mm, 5 μm; Mobile Phase A: Water (0.1% formic acid), Mobile Phase B: ACN; Flow rate: 25 mL/min; Gradient: 30% B to 55% B in 5.5 min, 55% B; RT=5.24 min.

### Example 79

### N-(1-(5-(3-cyano-6-ethoxypyrazolo[1,5-a]pyridin-4-yl)pyridin-2-yl)-4-((4-(1-(3-(2,4-dioxotetrahydropyrimidin-1(2H)-yl)-1-methyl-1H-indazol-6-yl)piperidine-4-carbonyl)piperazin-1-yl)methyl)piperidin-4-yl)-2,5-difluorobenzamide

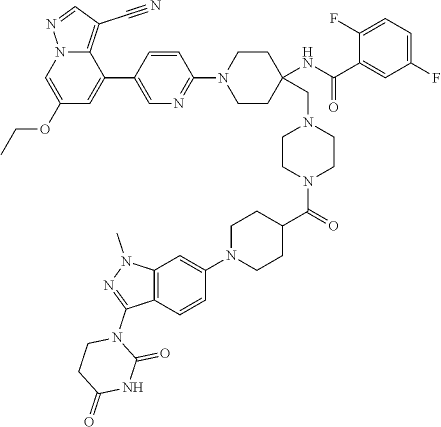 [SMILES: CCOc1cc(-c2ccc(N3CCC(CN4CCN(C(=O)C5CCN(c6ccc7c(N8CCC(=O)NC8=O)nn(C)c7c6)CC5)CC4)(NC(=O)c4cc(F)ccc4F)CC3)nc2)c2c(C#N)cnn2c1]

### A. Tert-butyl 1-[3-(2,4-dioxohexahydropyrimidin-1-yl)-1-methyl-indazol-6-yl]piperidine-4-carboxylate

The mixture solution of 1-(6-bromo-1-methyl-indazol-3-yl) hexahydropyrimidine-2,4-dione (300 mg, 0.93 mmol), tert-butyl piperidine-4-carboxylate (260 mg, 1.39 mmol), Ruphos (45 mg, 0.09 mmol), Ruphos Pd G3 (80 mg, 0.09 mmol) and Cs₂CO₃ (300 mg, 0.9300 mmol) in 1,4-Dioxane (8 mL) was purged with N₂ for 5 times, and the resulting mixture solution was warmed up to 100 °C and stirred overnight under N₂ atmosphere. The reaction was concentrated and purified by silica gel column to afford the desired product (210 mg, 52.9% yield) as a white solid. MS: m/z [M+H]⁺ 428.

### B. 1-[3-(2,4-dioxohexahydropyrimidin-1-yl)-1-methyl-indazol-6-yl]piperidine-4-carboxylic acid

To the mixture solution of tert-butyl 1-[3-(2,4-dioxohexahydropyrimidin-1-yl)-1-methyl-indazol-6-yl]piperidine-4-carboxylate (200 mg, 0.47 mmol) in DCM (10 mL) was added TFA (0.04 mL, 0.47 mmol), and stirred at rt for 4 h, the reaction was concentrated to afford the desired product (200 mg, quant.). MS: m/z [M+H]⁺ 372.

### C. N-[1-[5-(3-cyano-6-ethoxy-pyrazolo[1,5-a]pyridin-4-yl)-2-pyridyl]-4-[[4-[1-[3-(2,4-dioxohexahydropyrimidin-1-yl)-1-methyl-indazol-6-yl]piperidine-4-carbonyl]piperazin-1-yl]methyl]-4-piperidyl]-2,5-difluoro-benzamide

To a solution of 1-[3-(2,4-dioxohexahydropyrimidin-1-yl)-1-methyl-indazol-6-yl]piperidine-4-carboxylic acid (30 mg, 0.08 mmol) in DMF (3 mL) were added N-[1-[5-(3-cyano-6-ethoxy-pyrazolo[1,5-a]pyridin-4-yl)-2-pyridyl]-4-(piperazin-1-ylmethyl)-4-piperidyl]-2,5-difluoro-benzamide (50 mg, 0.08 mmol), DIEA (0.02 mL, 0.24 mmol), and HATU (50 mg, 0.12 mmol). The resulting solution was stirred for 1 h and purified by Prep-HPLC to afford the titled compound (19.2 mg, 24.8% yield) as a white solid. ¹H NMR (400 MHz, DMSO-d6) δ 10.51 (s, 1H), 8.66 (d, J=2.1 Hz, 1H), 8.58 (s, 1H), 8.34 (d, J=2.6 Hz, 1H), 8.16 (s, 1H), 7.77 (dd, J=8.9, 2.6 Hz, 1H), 7.48-7.42 (m, 1H), 7.40-7.32 (m, 3H), 7.28 (d, J=2.1 Hz, 1H), 7.02-2.95 (m, 1H), 6.93-6.89 (m, 1H), 6.84-6.80 (m, 1H), 4.25-4.13 (m, 4H), 3.93-3.87 (m, 5H), 3.86-3.79 (m, 2H), 3.56-3.50 (m, 2H), 3.48-3.41 (m, 2H), 3.23-3.11 (m, 2H), 2.88-2.79 (m, 3H), 2.78-2.70 (m, 4H), 2.60-2.53 (m, 5H), 2.40-2.35 (m, 1H), 1.75-1.57 (m, 6H), 1.39 (t, J=6.9 Hz, 3H). Analytical LCMS ESI-MS(+) m/z 954.6 [M+H]⁺, RT=1.53 min, purity: 99.7% (Method P). Prep-HPLC purification condition: Column: Xselect CSH C18 OBD Column 30×150 mm 5 μm; Mobile Phase A: Water (10 mmol/L NH₄HCO₃), Mobile Phase B: ACN; Flow rate: 60 mL/min; Gradient: 42% B to 58% B in 7 min, 58% B to 71% B in 8.5 min, 71% B; RT=7.58 min.

### Example 80

### N-(1-(5-(3-cyano-6-ethoxypyrazolo[1,5-a]pyridin-4-yl)pyridin-2-yl)-4-((4-(3-(4-(2-(3-methyl-2,6-dioxopiperidin-3-yl)-3-oxoisoindolin-5-yl)piperidin-1-yl)propanoyl)piperazin-1-yl)methyl)piperidin-4-yl)-2,5-difluorobenzamide

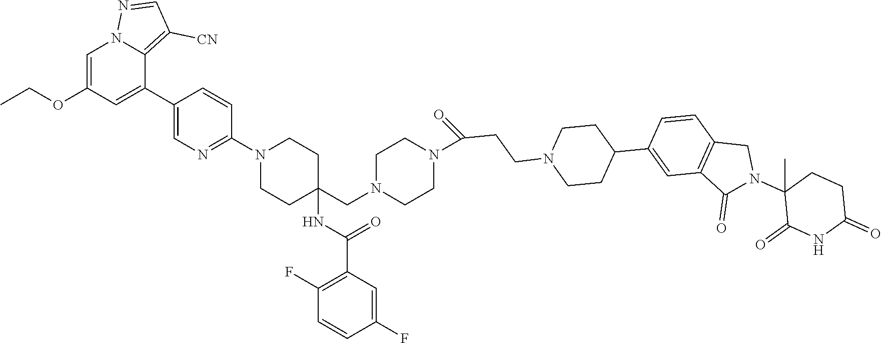 [SMILES: CCOc1cc(-c2ccc(N3CCC(CN4CCN(C(=O)CCN5CCC(c6ccc7c(c6)C(=O)N(C6(C)CCC(=O)NC6=O)C7)CC5)CC4)(NC(=O)c4cc(F)ccc4F)CC3)nc2)c2c(C#N)cnn2c1]

### A. 3-(6-Bromo-1-oxoisoindolin-2-yl)-3-methylpiperidine-2,6-dione

To a stirred mixture of methyl 5-bromo-2-(bromomethyl)benzoate (600 mg, 1.95 mmol) and 3-amino-3-methyl-piperidine-2,6-dione (332.35 mg, 2.34 mmol) in MeCN (8 mL) was added DIEA (0.2 mL, 7.79 mmol), and the resulting mixture was stirred at 60 °C for overnight. AcOH (0 mL, 0 mmol) was added to the reaction mixture, and the above mixture was stirred for 1 h at 80 °C. The suspension was filtrated, and the filter cake was dried to obtain the desired product (450 mg, 68.5% yield) as a white solid. MS: m/z [M+H]⁺ 337.

### B. Tert-butyl 4-(2-(3-methyl-2,6-dioxopiperidin-3-yl)-3-oxoisoindolin-5-yl)-3,6-dihydropyridine-1(2H)-carboxylate

To a solution of 3-(6-bromo-1-oxo-isoindolin-2-yl)-3-methyl-piperidine-2,6-dione (430 mg, 1.28 mmol), tert-butyl 4-(4,4,5,5-tetramethyl-1,3,2-dioxaborolan-2-yl)-3,6-dihydro-2H-pyridine-1-carboxylate (591.51 mg, 1.91 mmol) and Na₂CO₃ (405.55 mg, 3.83 mmol) in 1,4-dioxane (10 mL) and Water (1 mL) was added Pd(dppf)Cl₂ (208.3 mg, 0.26 mmol). The resulting mixture was evacuated and backfilled with N₂ for 3 times and stirred at 80 °C for 3 h. The suspension was filtrated, and the filtrate was concentrated under vacuum. The resulting residue was purified by Prep-TLC (PE/EA=0/1) to obtain the desired product (120 mg, 21.4% yield) as a tan solid. MS: m/z [M+H]⁺ 441.

### C. Tert-butyl 4-(2-(3-methyl-2,6-dioxopiperidin-3-yl)-3-oxoisoindolin-5-yl)piperidine-1-carboxylate

To a stirred solution of tert-butyl 4-[2-(3-methyl-2,6-dioxo-3-piperidyl)-3-oxo-isoindolin-5-yl]-3,6-dihydro-2H-pyridine-1-carboxylate (330 mg, 0.7500 mmol) in ethanol (3 mL) was added Pd/C (330 mg, 3.1 mmol) under N₂. H₂ was subsequently introduced into the reaction system, and the resulting mixture was stirred at ambient temperature for 1 h. The suspension was filtrated, and the filtrate was concentrated under vacuum. The resulting residue was dried to obtain the desired product (300 mg) as a white solid. MS: m/z [M−H]⁻ 440.

### D. 3-Methyl-3-(1-oxo-6-(piperidin-4-yl) isoindolin-2-yl)piperidine-2,6-dione

To a stirred solution of tert-butyl 4-[2-(3-methyl-2,6-dioxo-3-piperidyl)-3-oxo-isoindolin-5-yl]piperidine-1-carboxylate (290 mg, 0.66 mmol) in 1,4-dioxane (10 mL) was added a solution of 4 M HCl in 1,4-Dioxane (10 mL), the reaction mixture was stirred for 2 h at rt. The mixture was concentrated under vacuum to afford the desired product (290 mg, 0.66 mmol) as a white solid. MS: m/z [M+H]⁺ 342.

### E. Tert-butyl 3-(4-(2-(3-methyl-2,6-dioxopiperidin-3-yl)-3-oxoisoindolin-5-yl)piperidin-1-yl)propanoate

To a stirred mixture of 3-methyl-3-[1-oxo-6-(4-piperidyl) isoindolin-2-yl]piperidine-2,6-dione (100 mg, 0.29 mmol) and tert-butyl prop-2-enoate (75.08 mg, 0.59 mmol) in MeCN (3 mL) was added DBU (178.37 mg, 1.17 mmol) at rt, the reaction mixture was stirred for 4 h. It was concentrated and purified by reversed-phase column to obtain the desired product (90 mg, 65.4% yield) as a white solid.

### F. 3-(4-(2-(3-Methyl-2,6-dioxopiperidin-3-yl)-3-oxoisoindolin-5-yl)piperidin-1-yl)propanoic acid

To a stirred solution of tert-butyl 3-[4-[2-(3-methyl-2,6-dioxo-3-piperidyl)-3-oxo-isoindolin-5-yl]-1-piperidyl]propanoate (85 mg, 0.18 mmol) in DCM (5 mL) was added TFA (5 mL) at rt and stirred for 2 h. The mixture was concentrated under vacuum to obtain a crude product, which was not purified and used directly in the next step. MS: m/z [M+H]⁺ 414.

### G. N-(1-(5-(3-cyano-6-ethoxypyrazolo[1,5-a]pyridin-4-yl)pyridin-2-yl)-4-((4-(3-(4-(2-(3-methyl-2,6-dioxopiperidin-3-yl)-3-oxoisoindolin-5-yl)piperidin-1-yl)propanoyl)piperazin-1-yl)methyl)piperidin-4-yl)-2,5-difluorobenzamide

To a solution of N-[1-[5-(3-cyano-6-ethoxy-pyrazolo[1,5-a]pyridin-4-yl)-2-pyridyl]-4-(piperazin-1-ylmethyl)-4-piperidyl]-2,5-difluoro-benzamide (50 mg, 0.08 mmol) and 3-[4-[2-(3-methyl-2,6-dioxo-3-piperidyl)-3-oxo-isoindolin-5-yl]-1-piperidyl]propanoic acid (51.63 mg, 0.12 mmol) in DMF (2 mL) were added T₃P (52.94 mg, 0.17 mmol), and DIEA (0.02 mL, 0.25 mmol) at 0 °C. The reaction mixture was stirred at rt for 2 h. The mixture was purified by reversed phase column to obtain the titled product (18.4 mg, 22.1% yield) as a white solid. ¹H NMR (400 MHz, DMSO-d6) δ 10.87 (s, 1H), 9.35 (s, 1H), 8.67 (d, J=2.1 Hz, 1H), 8.58 (s, 1H), 8.35 (d, J=2.6 Hz, 1H), 7.81 (dd, J=8.9, 2.6 Hz, 1H), 7.69-7.35 (m, 6H), 7.28 (d, J=2.1 Hz, 1H), 7.05 (d, J=9.0 Hz, 1H), 4.80-4.60 (m, 2H), 4.26 (d, J=13.2 Hz, 2H), 4.16 (q, J=6.9 Hz, 2H), 3.50-3.45 (m, 2H), 3.36 (s, 4H), 3.21 (t, J=12.4 Hz, 3H), 3.11 (d, J=12.0 Hz, 3H), 2.98-2.82 (m, 5H), 2.81-2.60 (m, 4H), 2.55 (t, J=4.3 Hz, 2H), 2.42 (d, J=12.7 Hz, 2H), 2.04 (d, J=13.4 Hz, 2H), 2.00-1.84 (m, 3H), 1.83-1.60 (m, 5H), 1.39 (t, J=6.9 Hz, 3H). MS: m/z [M+H]⁺ 996. Prep-HPLC purification condition: Column: Aeris PEPTIDE 5 μm XB—C18 Axia, 21.2 mm×250 mm, 5 μm; Mobile Phase A: Water (0.05% TFA), Mobile Phase B: ACN; Flow rate: 25 mL/min; Gradient: 25% B to 50% B in 6.5 min, 50% B; RT=6.75 min.

### Example 81

### (S)—N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)propanoyl)piperazin-1-yl)methyl)piperidin-4-yl)-2,5-difluorobenzamide

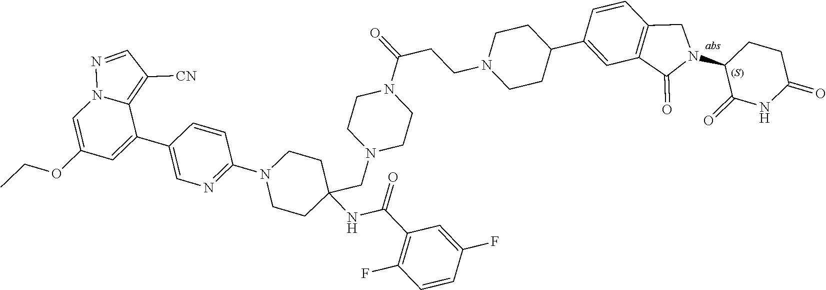

### A. Tert-butyl (4S)-5-amino-4-(6-bromo-1-oxo-isoindolin-2-yl)-5-oxo-pentanoate

To a solution of methyl 5-bromo-2-(bromomethyl)benzoate (1 g, 3.25 mmol), tert-butyl (4S)-4,5-diamino-5-oxo-pentanoate, hydrochloride (775.11 mg, 3.25 mmol) in DMF (30 mL) was added DIEA (1.74 mL, 9.74 mmol). The mixture was stirred at 60 °C for 16 h. It was cooled to rt, water was used to quench the reaction, extracted with EtOAc, washed with brine, combined the organic phase, dried over anhydrous sodium sulphate, filtered, concentrated, and purified by column chromatography to afford the desired product (1.2 g, 93% yield) as a white solid. MS: m/z [M+H]⁺ 397.

### B. Benzyl 4-[2-[(1S)-4-tert-butoxy-1-carbamoyl-4-oxo-butyl]-3-oxo-isoindolin-5-yl]-3,6-dihydro-2H-pyridine-1-carboxylate

To a 30 mL microwave vial, tert-butyl (4S)-5-amino-4-(6-bromo-1-oxo-isoindolin-2-yl)-5-oxo-pentanoate (330 mg, 0.83 mmol), benzyl 4-(4,4,5,5-tetramethyl-1,3,2-dioxaborolan-2-yl)-3,6-dihydro-2H-pyridine-1-carboxylate (570.24 mg, 1.66 mmol), Pd(dppf)Cl₂ (67.84 mg, 0.08 mmol), Na₂CO₃ (264.16 mg, 2.49 mmol) were suspended in 1,4-dioxane (20 mL) and Water (2 mL). The reaction was placed under vacuum, sonicated and backfilled with nitrogen. The resulting mixture was stirred at 100 °C for 4 h. It was concentrated, the crude was purified by column chromatography (PE:EtOAc=1:1 to EtOAc) to afford the desired product (400 mg, 90% yield) as a yellow solid. MS: m/z [M+H]⁺ 534.

### C. Tert-butyl (4S)-5-amino-5-oxo-4-[1-oxo-6-(4-piperidyl) isoindolin-2-yl]pentanoate

A solution of benzyl 4-[2-[(1S)-4-tert-butoxy-1-carbamoyl-4-oxo-butyl]-3-oxo-isoindolin-5-yl]-3,6-dihydro-2H-pyridine-1-carboxylate (600 mg, 1.12 mmol) in Ethanol (20 mL) was placed under vacuum, sonicated and backfilled with nitrogen. Pd/C (100 mg) was added under N₂, and the reaction was placed under vacuum, sonicated and backfilled with H₂. The mixture was stirred at rt for 4 h. It was filtered, washed with EtOH, concentrated to afford crude product (400 mg, 88.6% yield) as a yellow solid. MS: m/z [M+H]⁺ 402.

### D. Tert-butyl (4S)-5-amino-4-[6-[1-(3-benzyloxy-3-oxo-propyl)-4-piperidyl]-1-oxo-isoindolin-2-yl]-5-oxo-pentanoate

To a solution of tert-butyl (4S)-5-amino-5-oxo-4-[1-oxo-6-(4-piperidyl) isoindolin-2-yl]pentanoate (200 mg, 0.50 mmol), benzyl prop-2-enoate (161.58 mg, 1 mmol) in MeCN (1 mL) was added DBU (0.22 mL, 1.49 mmol). The mixture was stirred at rt for 4 h. It was concentrated and purified by reversed phase column chromatography to afford the desired product (180 mg, 64% yield) as a yellow oil. MS: m/z [M+H]⁺ 564.

### E. (S)-3-(4-(2-(1-Amino-5-(tert-butoxy)-1,5-dioxopentan-2-yl)-3-oxoisoindolin-5-yl)piperidin-1-yl)propanoic acid

To a solution of tert-butyl (4S)-5-amino-4-[6-[1-(3-benzyloxy-3-oxo-propyl)-4-piperidyl]-1-oxo-isoindolin-2-yl]-5-oxo-pentanoate (175 mg, 0.31 mmol) in Ethanol (6 mL) was added Pd/C (30 mg) under N₂. The reaction was placed under vacuum, sonicated and backfilled with H₂. The mixture was stirred at rt for 6 h. It was filtered and concentrated to afford the crude product, which was used directly in the next step without further purification. MS: m/z [M+H]⁺ 474.

### F. Tert-butyl (4S)-5-amino-4-[6-[1-[3-[4-[[1-[5-(3-cyano-6-ethoxy-pyrazolo[1,5-a]pyridin-4-yl)-2-pyridyl]-4-[(2,5-difluorobenzoyl)amino]-4-piperidyl]methyl]piperazin-1-yl]-3-oxo-propyl]-4-piperidyl]-1-oxo-isoindolin-2-yl]-5-oxo-pentanoate

To a solution of N-[1-[5-(1-cyano-6-ethoxy-indolizin-8-yl)-2-pyridyl]-4-(piperazin-1-ylmethyl)-4-piperidyl]-2,5-difluoro-benzamide (80 mg, 0.13 mmol), 3-[4-[2-[(1S)-4-tert-butoxy-1-carbamoyl-4-oxo-butyl]-3-oxo-isoindolin-5-yl]-1-piperidyl]propanoic acid (63.18 mg, 0.13 mmol) in DMA (1 mL) were added HATU (76.09 mg, 0.20 mmol), and DIEA (51.63 mg, 0.40 mmol). The mixture was stirred at rt for 2 h. The crude was purified by reversed phase column chromatography to afford the desired product (80 mg, 56.8% yield) as an off-white solid. MS: m/z [M+H]⁺ 1057.

### G. N-[1-[5-(3-cyano-6-ethoxy-pyrazolo[1,5-a]pyridin-4-yl)-2-pyridyl]-4-[[4-[3-[4-[2-[(3S)-2,6-dioxo-3-piperidyl]-3-oxo-isoindolin-5-yl]-1-piperidyl]propanoyl]piperazin-1-yl]methyl]-4-piperidyl]-2,5-difluoro-benzamide

To a solution of tert-butyl (4S)-5-amino-4-[6-[1-[3-[4-[[1-[5-(3-cyano-6-ethoxy-pyrazolo[1,5-a]pyridin-4-yl)-2-pyridyl]-4-[(2,5-difluorobenzoyl)amino]-4-piperidyl]methyl]piperazin-1-yl]-3-oxo-propyl]-4-piperidyl]-1-oxo-isoindolin-2-yl]-5-oxo-pentanoate (70 mg, 0.07 mmol) in MeCN (6 mL) was added triphosgene (62.9 mg, 0.40 mmol). The mixture was stirred at 60 °C for 20 h. The reaction was concentrated to afford the crude, which was purified by Prep-HPLC to afford the titled compound (49.4 mg, 74.6% yield) as a white solid. ¹H NMR (400 MHz, DMSO-d6) δ 11.00 (s, 1H), 9.45 (s, 1H), 8.67 (d, J=2.1 Hz, 1H), 8.59 (s, 1H), 8.36 (d, J=2.5 Hz, 1H), 7.83 (dd, J=8.9, 2.5 Hz, 1H), 7.61-7.59 (m, 2H), 7.55-7.47 (m, 2H), 7.43-7.37 (m, 2H), 7.29 (d, J=2.1 Hz, 1H), 7.07 (d, J=8.9 Hz, 1H), 5.01 (dd, J=13.3, 5.1 Hz, 1H), 4.51-4.42 (m, 1H), 4.43-4.41 (m, 1H), 4.34-4.32 (m, 1H), 4.29-4.24 (m, 2H), 3.92-3.71 (m, 3H), 3.68-3.61 (m, 6H), 3.40-3.35 (m, 3H), 3.24-3.09 (m, 6H), 2.99-2.88 (m, 5H), 2.65-2.57 (m, 1H), 2.45-2.38 (m, 3H), 2.11-1.93 (m, 5H), 1.79-1.68 (m, 2H), 1.37 (t, J=6.9 Hz, 3H). Analytical LCMS ESI-MS(+) m/z 982.5 [M+H]⁺, RT=1.28 min, purity: 100% (Method Q). Prep-HPLC purification condition: Column: SunFire Prep C18 OBD Column, 19×150 mm, 5 μm; Mobile Phase A: Water (0.05% TFA), Mobile Phase B: ACN; Flow rate: 25 mL/min; Gradient: 20% B to 50% B in 6.5 min, 50% B; RT=5.68 min. ee %=96.6%, Analytical SFC: CHIRALPAK IA-3 3.0×50 mm; 3 μm, Co-Solvent: EtOH:DCM=1: 1 (0.5% 2M NH₃-MeOH). Alternatively, the titled compound was obtained by chiral separation of Example 17.

### Example 82

### (R)—N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)propanoyl)piperazin-1-yl)methyl)piperidin-4-yl)-2,5-difluorobenzamide

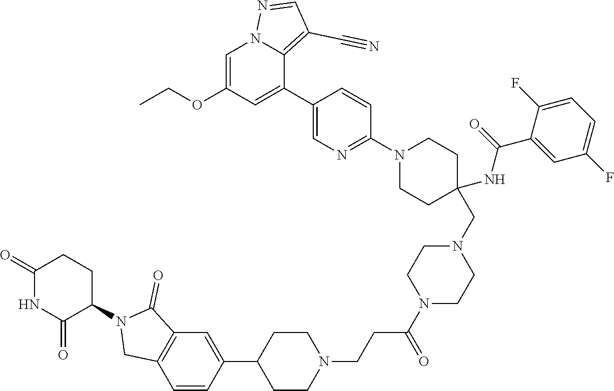 [SMILES: CCOc1cc(-c2ccc(N3CCC(CN4CCN(C(=O)CCN5CCC(c6ccc7c(c6)C(=O)N([C@@H]6CCC(=O)NC6=O)C7)CC5)CC4)(NC(=O)c4cc(F)ccc4F)CC3)nc2)c2c(C#N)cnn2c1]

and

### Example 81

### (S)—N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)propanoyl)piperazin-1-yl)methyl)piperidin-4-yl)-2,5-difluorobenzamide

Racemic Example 17 (50.0 mg) was purified via chiral preparative SFC chromatography with the following conditions: Column: Chiralcel OJ-H, 30 mm×250 mm, 5 μm particles; Mobile Phase A: CO2; Mobile Phase B: EtOH with 0.1% NH4OH/0.1% formic acid; Isocratic elution at 38% B over 42 min; Flow Rate: 85 mL/min; Column Temperature: 45 °C. Fraction collection was triggered by UV (220 nm). Fractions containing the desired product were combined and dried via centrifugal evaporation. Chiral analytical SFC was used to determine the final purity. Conditions: Column: Chiralcel OJ-H, 4.6 mm×150 mm, 5 μm particles; Mobile Phase A: CO2; Mobile Phase B: EtOH; Temperature: 45 °C; Isocratic elution at 40% B over 20 min; Flow: 2 mL/min; Detection: UV (220 nm) and MS (ESI+).

Concentration of the appropriate (earlier eluting) fractions afforded Example 82 (16.5 mg) assigned as (R)—N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)propanoyl)piperazin-1-yl)methyl)piperidin-4-yl)-2,5-difluorobenzamide (Enantiomer 1). Chiral analytical SFC Tᵣ=8.347 min; Purity=99% (% ee=100%). Analytical LCMS: m/z 982.3 [M+H]⁺, RT=1.232 min (UHPLC Method D). Concentration of the appropriate (later eluting) fractions afforded Example 81 (20.9 mg) assigned as(S)—N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)propanoyl)-piperazin-1-yl)methyl)piperidin-4-yl)-2,5-difluorobenzamide (Enantiomer 2). Chiral analytical SFC Tᵣ=9.583 min; Purity=95.2% (% ee=90.3%). Analytical LCMS: m/z 982.6 [M+H]⁺, RT=1.239 min (UHPLC Method D).

### Example 83

### N-(1-(5-(3-cyano-6-ethoxypyrazolo[1,5-a]pyridin-4-yl)pyridin-2-yl)-4-((4-(6-(4-(2-(2,6-dioxopiperidin-3-yl)-1-oxoisoindolin-5-yl)piperazin-1-yl)spiro[3.3]heptane-2-carbonyl)piperazin-1-yl)methyl)piperidin-4-yl)-2,5-difluorobenzamide

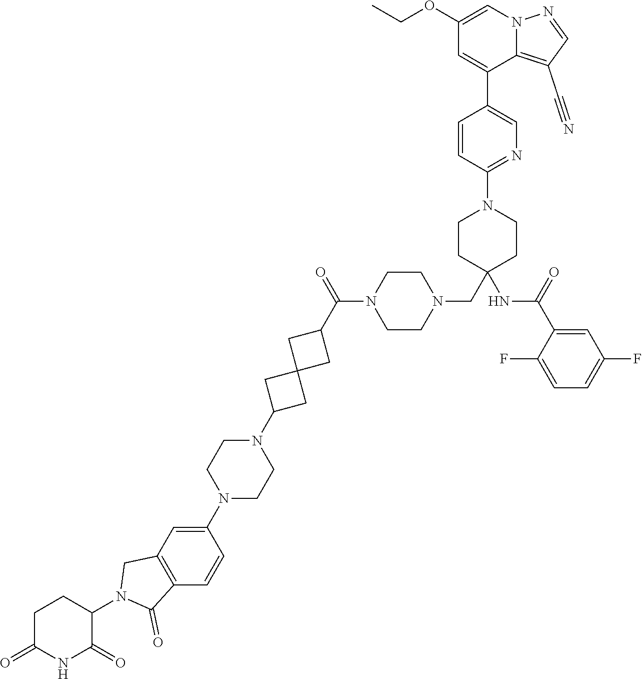 [SMILES: CCOc1cc(-c2ccc(N3CCC(CN4CCN(C(=O)C5CC6(C5)CC(N5CCN(c7ccc8c(c7)CN(C7CCC(=O)NC7=O)C8=O)CC5)C6)CC4)(NC(=O)c4cc(F)ccc4F)CC3)nc2)c2c(C#N)cnn2c1]

A solution of 3-(1-oxo-5-(piperazin-1-yl) isoindolin-2-yl)piperidine-2,6-dione (20.05 mg, 0.061 mmol), N-(1-(5-(3-cyano-6-ethoxypyrazolo[1,5-a]pyridin-4-yl)pyridin-2-yl)-4-((4-(6-oxospiro[3.3]heptane-2-carbonyl)piperazin-1-yl)methyl)piperidin-4-yl)-2,5-difluorobenzamide (45 mg, 0.061 mmol), and AcOH (6.99 μl, 0.122 mmol) in DCE (2 mL) is heated to 80 °C with stirring. After 6 h the reaction is cooled to rt and sodium triacetoxyborohydride (38.8 mg, 0.183 mmol) was added. After 16 h the reaction is evaporated under reduced pressure and dried under high vacuum. The reaction mixture is diluted with DMF:Acetic acid (1:12 mL). The crude material was purified via preparative Reverse Phase chromatography to yield the titled compound (28 mg, 40% yield). Analytical LCMS ESI-MS(+) m/z 1049.6 [M+H]⁺, RT=1.31 min, purity: 100% (Method Q). ¹H NMR (500 MHz, DMSO-d6) δ 10.98-10.90 (m, 1H), 8.67-8.61 (m, 1H), 8.60-8.54 (m, 1H), 8.35-8.29 (m, 1H), 8.15-8.08 (m, 1H), 7.79-7.73 (m, 1H), 7.56-7.48 (m, 1H), 7.39-7.30 (m, 3H), 7.30-7.24 (m, 1H), 7.10-7.03 (m, 1H), 7.01-6.92 (m, 1H), 5.11-4.98 (m, 1H), 4.38-4.29 (m, 1H), 4.45-4.06 (m, 5H), 3.24-3.11 (m, 2H), 2.96-2.84 (m, 1H), 2.75-2.69 (m, 2H), 2.64-2.56 (m, 1H), 2.56-2.53 (m, 3H), 2.50-2.42 (m, 4H), 2.44-2.37 (m, 2H), 2.37-2.30 (m, 2H), 2.30-2.22 (m, 1H), 2.21-2.16 (m, 1H), 2.15-2.07 (m, 1H), 2.07-1.93 (m, 3H), 1.92-1.83 (m, 1H), 1.82-1.72 (m, 1H), 1.68-1.56 (m, 2H), 1.43-1.35 (m, 3H) 6 methylenes are missing due to water suppression.

Examples 84-99 were prepared using procedures similar to the ones described in the aforementioned examples.

### Example 84

### 6-(4-{[1-(5-{3-cyano-6-ethoxypyrazolo[1,5-a]pyridin-4-yl}pyridin-2-yl)-4-(2,5-difluorobenzamido)piperidin-4-yl]methyl}piperazine-1-carbonyl)-N-[3-(2,4-dioxo-1,3-diazinan-1-yl)-1-methyl-1H-indazol-6-yl]spiro[3.3]heptane-2-carboxamide

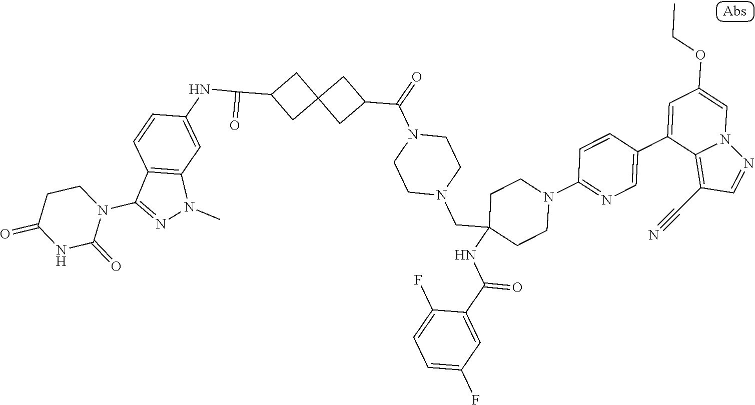 [SMILES: CCOc1cc(-c2ccc(N3CCC(CN4CCN(C(=O)C5CC6(CC(C(=O)Nc7ccc8c(N9CCC(=O)NC9=O)nn(C)c8c7)C6)C5)CC4)(NC(=O)c4cc(F)ccc4F)CC3)nc2)c2c(C#N)cnn2c1]

### Example 85

### N-[1-(5-{3-cyano-6-ethoxypyrazolo[1,5-a]pyridin-4-yl}pyridin-2-yl)-4-{[4-(6-{4-[2-(2,6-dioxopiperidin-3-yl)-1-oxo-2,3-dihydro-1H-isoindol-5-yl]piperazine-1-carbonyl}spiro[3.3]heptane-2-carbonyl)piperazin-1-yl]methyl}piperidin-4-yl]-2,5-difluorobenzamide

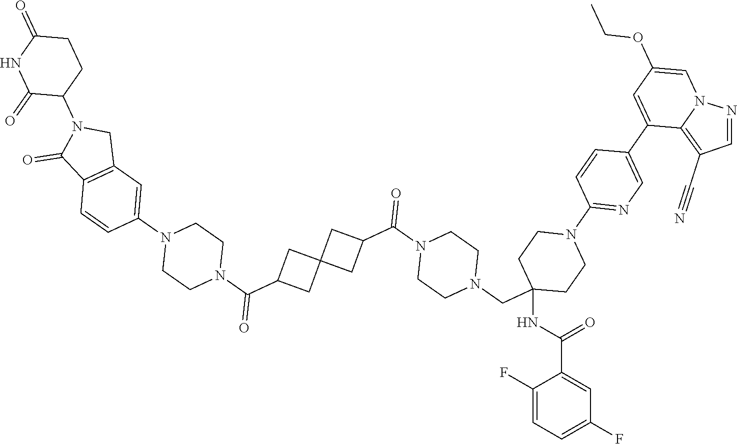 [SMILES: CCOc1cc(-c2ccc(N3CCC(CN4CCN(C(=O)C5CC6(C5)CC(C(=O)N5CCN(c7ccc8c(c7)CN(C7CCC(=O)NC7=O)C8=O)CC5)C6)CC4)(NC(=O)c4cc(F)ccc4F)CC3)nc2)c2c(C#N)cnn2c1]

### Example 86

### N-[1-(5-{3-cyano-6-ethoxypyrazolo[1,5-a]pyridin-4-yl}pyridin-2-yl)-4-{[4-(6-{4-[2-(2,6-dioxopiperidin-3-yl)-3-oxo-2,3-dihydro-1H-isoindol-5-yl]piperazine-1-carbonyl}spiro[3.3]heptane-2-carbonyl)piperazin-1-yl]methyl}piperidin-4-yl]-2,5-difluorobenzamide

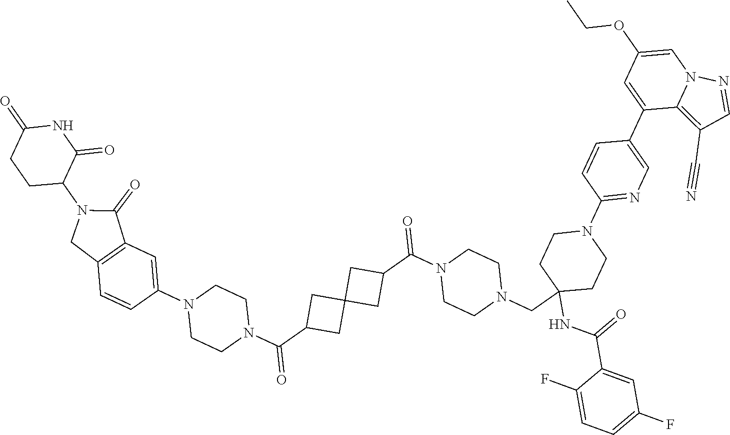 [SMILES: CCOc1cc(-c2ccc(N3CCC(CN4CCN(C(=O)C5CC6(C5)CC(C(=O)N5CCN(c7ccc8c(c7)C(=O)N(C7CCC(=O)NC7=O)C8)CC5)C6)CC4)(NC(=O)c4cc(F)ccc4F)CC3)nc2)c2c(C#N)cnn2c1]

### Example 87

### N-[1-(5-{3-cyano-6-ethoxypyrazolo[1,5-a]pyridin-4-yl}pyridin-2-yl)-4-{[9-(3-{4-[2-(2,6-dioxopiperidin-3-yl)-1-oxo-2,3-dihydro-1H-isoindol-5-yl]piperazin-1-yl}propanoyl)-3,9-diazaspiro[5.5]undecan-3-yl]methyl}piperidin-4-yl]-2,5-difluorobenzamide

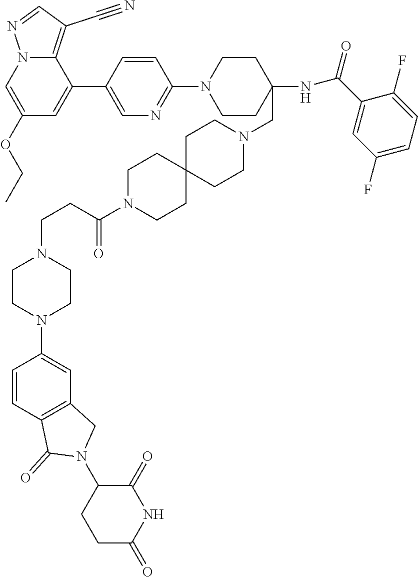 [SMILES: CCOc1cc(-c2ccc(N3CCC(CN4CCC5(CC4)CCN(C(=O)CCN4CCN(c6ccc7c(c6)CN(C6CCC(=O)NC6=O)C7=O)CC4)CC5)(NC(=O)c4cc(F)ccc4F)CC3)nc2)c2c(C#N)cnn2c1]

### Example 88

### N-[1-(5-{3-cyano-6-ethoxypyrazolo[1,5-a]pyridin-4-yl}pyridin-2-yl)-4-{[9-(3-{4-[2-(2,6-dioxopiperidin-3-yl)-1-oxo-2,3-dihydro-1H-isoindol-5-yl]piperidin-1-yl}propanoyl)-3,9-diazaspiro[5.5]undecan-3-yl]methyl}piperidin-4-yl]-2,5-difluorobenzamide

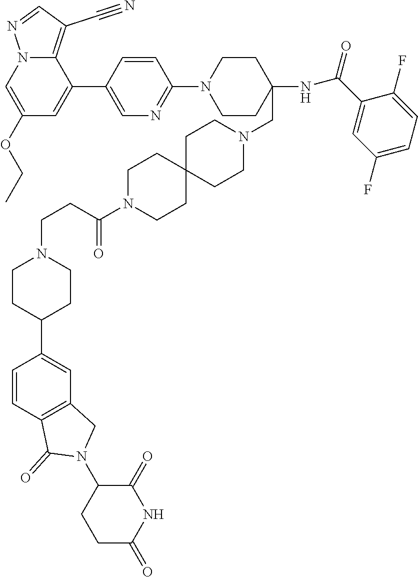 [SMILES: CCOc1cc(-c2ccc(N3CCC(CN4CCC5(CC4)CCN(C(=O)CCN4CCC(c6ccc7c(c6)CN(C6CCC(=O)NC6=O)C7=O)CC4)CC5)(NC(=O)c4cc(F)ccc4F)CC3)nc2)c2c(C#N)cnn2c1]

### Example 89

### N-[1-(5-{3-cyano-6-ethoxypyrazolo[1,5-a]pyridin-4-yl}pyridin-2-yl)-4-{[6-(3-{4-[2-(2,6-dioxopiperidin-3-yl)-1-oxo-2,3-dihydro-1H-isoindol-5-yl]piperidin-1-yl}propanoyl)-2,6-diazaspiro[3.3]heptan-2-yl]methyl}piperidin-4-yl]-2,5-difluorobenzamide

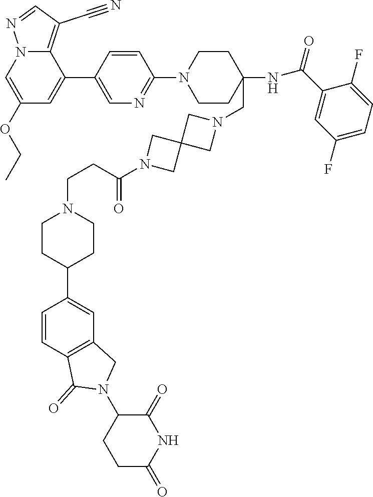 [SMILES: CCOc1cc(-c2ccc(N3CCC(CN4CC5(C4)CN(C(=O)CCN4CCC(c6ccc7c(c6)CN(C6CCC(=O)NC6=O)C7=O)CC4)C5)(NC(=O)c4cc(F)ccc4F)CC3)nc2)c2c(C#N)cnn2c1]

### Example 90

### N-[1-(5-{3-cyano-6-ethoxypyrazolo[1,5-a]pyridin-4-yl}pyridin-2-yl)-4-{[4-(3-{9-[2-(2,6-dioxopiperidin-3-yl)-1-oxo-2,3-dihydro-1H-isoindol-5-yl]-3,9-diazaspiro[5.5]undecan-3-yl}-3-oxopropyl)piperazin-1-yl]methyl}piperidin-4-yl]-2,5-difluorobenzamide

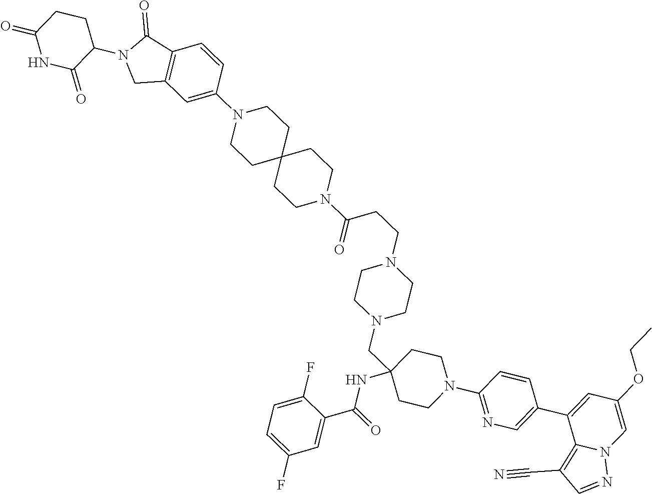 [SMILES: CCOc1cc(-c2ccc(N3CCC(CN4CCN(CCC(=O)N5CCC6(CC5)CCN(c5ccc7c(c5)CN(C5CCC(=O)NC5=O)C7=O)CC6)CC4)(NC(=O)c4cc(F)ccc4F)CC3)nc2)c2c(C#N)cnn2c1]

### Example 91

### N-[1-(5-{3-cyano-6-ethoxypyrazolo[1,5-a]pyridin-4-yl}pyridin-2-yl)-4-{[4-(3-{9-[2-(2,6-dioxopiperidin-3-yl)-1-oxo-2,3-dihydro-1H-isoindol-5-yl]-3,9-diazaspiro[5.5]undecan-3-yl}propanoyl)piperazin-1-yl]methyl}piperidin-4-yl]-2,5-difluorobenzamide

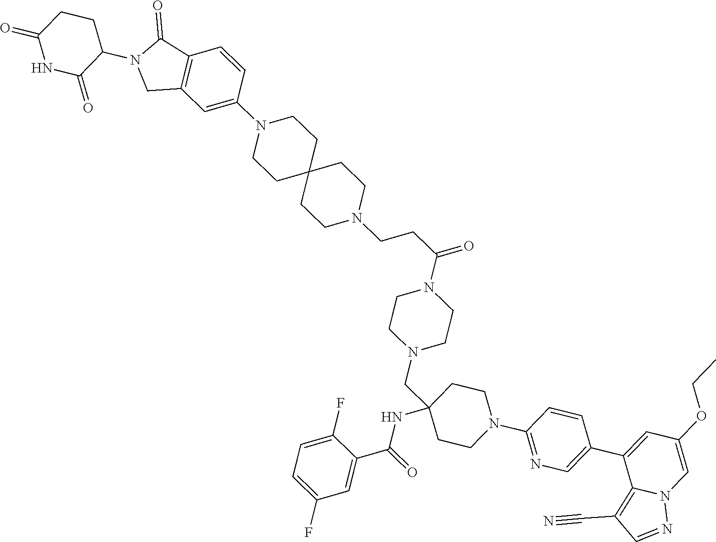 [SMILES: CCOc1cc(-c2ccc(N3CCC(CN4CCN(C(=O)CCN5CCC6(CC5)CCN(c5ccc7c(c5)CN(C5CCC(=O)NC5=O)C7=O)CC6)CC4)(NC(=O)c4cc(F)ccc4F)CC3)nc2)c2c(C#N)cnn2c1]

### Example 92

### N-[1-(5-{3-cyano-6-ethoxypyrazolo[1,5-a]pyridin-4-yl}pyridin-2-yl)-4-{[4-(6-{4-[2-(2,6-dioxopiperidin-3-yl)-1-oxo-2,3-dihydro-1H-isoindol-5-yl]piperidin-1-yl}spiro[3.3]heptane-2-carbonyl)piperazin-1-yl]methyl}piperidin-4-yl]-2,5-difluorobenzamide

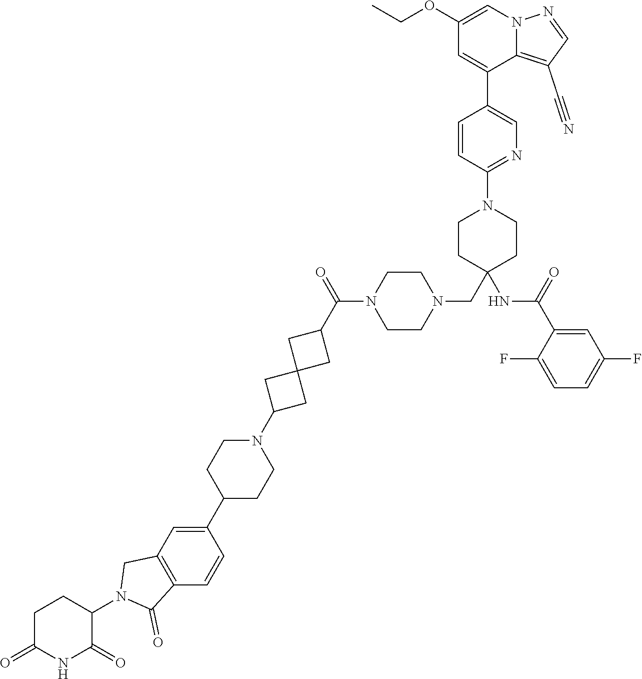 [SMILES: CCOc1cc(-c2ccc(N3CCC(CN4CCN(C(=O)C5CC6(C5)CC(N5CCC(c7ccc8c(c7)CN(C7CCC(=O)NC7=O)C8=O)CC5)C6)CC4)(NC(=O)c4cc(F)ccc4F)CC3)nc2)c2c(C#N)cnn2c1]

### Example 93

### N-[1-(5-{3-cyano-6-ethoxypyrazolo[1,5-a]pyridin-4-yl}pyridin-2-yl)-4-{[4-(6-{[3-(2,4-dioxo-1,3-diazinan-1-yl)-1-methyl-1H-indazol-6-yl]amino}spiro[3.3]heptane-2-carbonyl)piperazin-1-yl]methyl}piperidin-4-yl]-2,5-difluorobenzamide

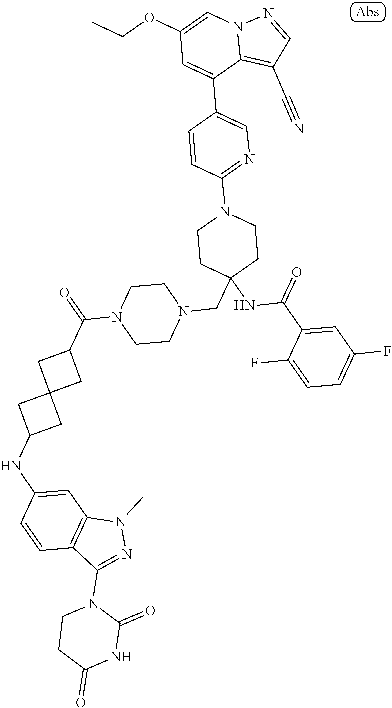 [SMILES: CCOc1cc(-c2ccc(N3CCC(CN4CCN(C(=O)C5CC6(CC(Nc7ccc8c(N9CCC(=O)NC9=O)nn(C)c8c7)C6)C5)CC4)(NC(=O)c4cc(F)ccc4F)CC3)nc2)c2c(C#N)cnn2c1]

### Example 94

### N-[1-(5-{3-cyano-6-ethoxypyrazolo[1,5-a]pyridin-4-yl}pyridin-2-yl)-4-{[4-(6-{4-[2-(2,6-dioxopiperidin-3-yl)-1-oxo-2,3-dihydro-1H-isoindol-5-yl]piperazine-1-carbonyl}spiro[3.3]heptan-2-yl)piperazin-1-yl]methyl}piperidin-4-yl]-2,5-difluorobenzamide

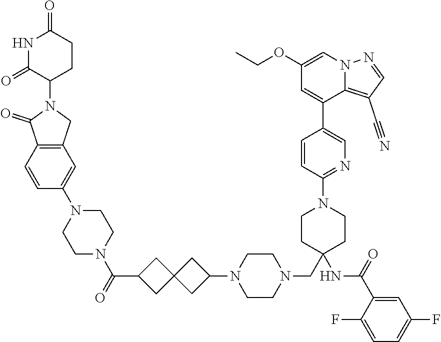 [SMILES: CCOc1cc(-c2ccc(N3CCC(CN4CCN(C5CC6(CC(C(=O)N7CCN(c8ccc9c(c8)CN(C8CCC(=O)NC8=O)C9=O)CC7)C6)C5)CC4)(NC(=O)c4cc(F)ccc4F)CC3)nc2)c2c(C#N)cnn2c1]

### Example 95

### N-[1-(5-{3-cyano-6-ethoxypyrazolo[1,5-a]pyridin-4-yl}pyridin-2-yl)-4-{[4-(6-{4-[2-(2,6-dioxopiperidin-3-yl)-1-oxo-2,3-dihydro-1H-isoindol-5-yl]piperidine-1-carbonyl}spiro[3.3]heptan-2-yl)piperazin-1-yl]methyl}piperidin-4-yl]-2,5-difluorobenzamide

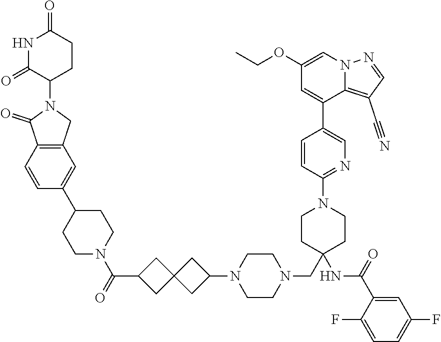 [SMILES: CCOc1cc(-c2ccc(N3CCC(CN4CCN(C5CC6(CC(C(=O)N7CCC(c8ccc9c(c8)CN(C8CCC(=O)NC8=O)C9=O)CC7)C6)C5)CC4)(NC(=O)c4cc(F)ccc4F)CC3)nc2)c2c(C#N)cnn2c1]

### Example 96

### N-[1-(5-{3-cyano-6-ethoxypyrazolo[1,5-a]pyridin-4-yl}pyridin-2-yl)-4-({9-[2-(2,6-dioxopiperidin-3-yl)-3-oxo-2,3-dihydro-1H-isoindole-5-carbonyl]-3,9-diazaspiro[5.5]undecan-3-yl}methyl)piperidin-4-yl]-2,5-difluorobenzamide

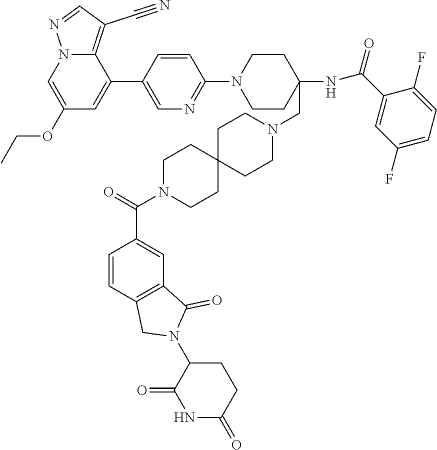 [SMILES: CCOc1cc(-c2ccc(N3CCC(CN4CCC5(CC4)CCN(C(=O)c4ccc6c(c4)C(=O)N(C4CCC(=O)NC4=O)C6)CC5)(NC(=O)c4cc(F)ccc4F)CC3)nc2)c2c(C#N)cnn2c1]

### Example 97

### N-[1-(5-{3-cyano-6-ethoxypyrazolo[1,5-a]pyridin-4-yl}pyridin-2-yl)-4-{[6-(2-{[2-(2,6-dioxopiperidin-3-yl)-1-oxo-2,3-dihydro-1H-isoindol-5-yl]oxy}acetyl)-2,6-diazaspiro[3.3]heptan-2-yl]methyl}piperidin-4-yl]-2,5-difluorobenzamide

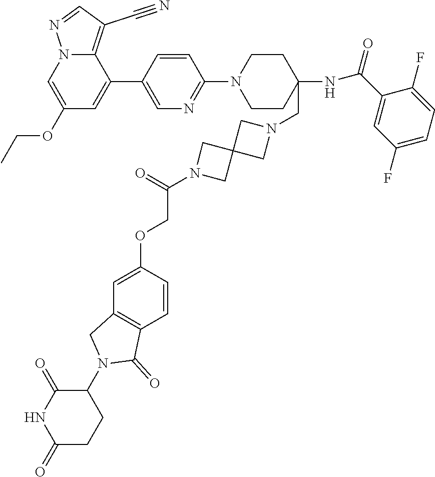 [SMILES: CCOc1cc(-c2ccc(N3CCC(CN4CC5(C4)CN(C(=O)COc4ccc6c(c4)CN(C4CCC(=O)NC4=O)C6=O)C5)(NC(=O)c4cc(F)ccc4F)CC3)nc2)c2c(C#N)cnn2c1]

### Example 98

### N-[1-(5-{3-cyano-6-ethoxypyrazolo[1,5-a]pyridin-4-yl}pyridin-2-yl)-4-[(9-{2-[2-(2,6-dioxopiperidin-3-yl)-1-oxo-2,3-dihydro-1H-isoindol-5-yl]acetyl}-3,9-diazaspiro[5.5]undecan-3-yl)methyl]piperidin-4-yl]-2,5-difluorobenzamide

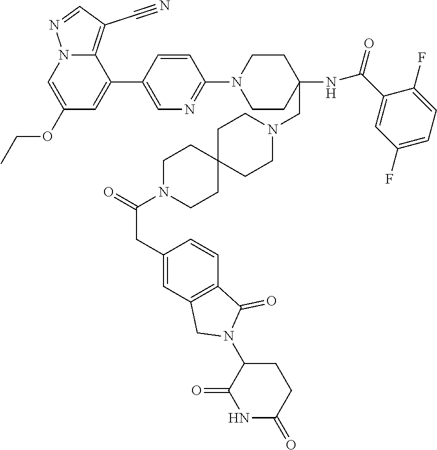 [SMILES: CCOc1cc(-c2ccc(N3CCC(CN4CCC5(CC4)CCN(C(=O)Cc4ccc6c(c4)CN(C4CCC(=O)NC4=O)C6=O)CC5)(NC(=O)c4cc(F)ccc4F)CC3)nc2)c2c(C#N)cnn2c1]

### Example 99

### N-[1-(5-{3-cyano-6-ethoxypyrazolo[1,5-a]pyridin-4-yl}pyridin-2-yl)-4-{[9-(2-{[2-(2,6-dioxopiperidin-3-yl)-1-oxo-2,3-dihydro-1H-isoindol-5-yl]oxy}acetyl)-3,9-diazaspiro[5.5]undecan-3-yl]methyl}piperidin-4-yl]-2,5-difluorobenzamide

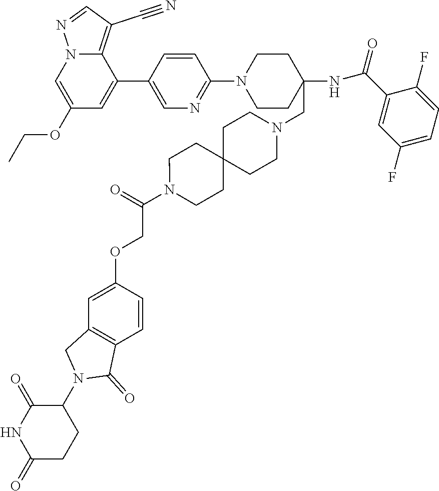 [SMILES: CCOc1cc(-c2ccc(N3CCC(CN4CCC5(CC4)CCN(C(=O)COc4ccc6c(c4)CN(C4CCC(=O)NC4=O)C6=O)CC5)(NC(=O)c4cc(F)ccc4F)CC3)nc2)c2c(C#N)cnn2c1]

| | Analytical | |
|---|---|---|
| | LCMS | |
| | ESI-MS(+) | |
| | m/z/RT | |
| | (min) | |
| Example # | (Method); | ¹H NMR |
| 84 | 1008 | ¹H NMR (500 MHz, DMSO-d₆) δ 10.59-10.47 (m, 1H), 10.03- |
| | [M + H]⁺, | 9.95 (m, 1H), 8.65-8.60 (m, 1H), 8.59-8.53 (m, 1H), 8.36-8.29 |
| | RT = 1.40 | (m, 1H), 8.17-8.07 (m, 2H), 7.80-7.71 (m, 1H), 7.58-7.51 (m, |
| | min (A) | 1H), 7.39-7.31 (m, 3H), 7.29-7.22 (m, 1H), 7.10-7.02 (m, 1H), |
| | | 7.00-6.92 (m, 1H), 4.24-4.10 (m, 4H), 3.96-3.87 (m, 5H), 3.23- |
| | | 3.07 (m, 3H), 2.78-2.70 (m, 4H), 2.56-2.53 (m, 6H), 2.50- |
| | | 2.44 (m, 3H), 2.39-2.21 (m, 6H), 2.19-2.02 (m, 4H), 1.68-1.56 |
| | | (m, 2H), 1.44-1.34 (m, 3H) |
| 85 | 177 | 1H NMR (500 MHz, DMSO-d6) Shift 11.03-10.81 (m, 1H), 8.69- |
| | [M + H]⁺, | 8.61 (m, 1H), 8.57 (s, 1H), 8.48 (br s, 1H), 8.36 (d, J = 2.3 Hz, |
| | RT = 1.98 | 1H), 7.83 (dd, J = 8.9, 2.3 Hz, 1H), 7.55 (d, J = 8.4 Hz, 1H), 7.53- |
| | min (A) | 7.46 (m, 1H), 7.46-7.35 (m, 2H), 7.27 (d, J = 1.8 Hz, 1H), 7.11- |
| | | 7.00 (m, 3H), 5.04 (dd, J = 13.2, 5.1 Hz, 1H), 4.42-4.10 (m, 6H), |
| | | 3.33-3.13 (m, 5H), 2.97-2.83 (m, 1H), 2.66-2.56 (m, 1H), 2.48- |
| | | 2.31 (m, 3H), 2.31-2.18 (m, 4H), 2.16-2.02 (m, 4H), 2.01- |
| | | 1.92 (m, 1H), 1.82-1.63 (m, 2H), 1.45-1.34 (m, 3H) 6 |
| | | methylenes missing due to water suppression and DMSO |
| | | overlapping |
| 86 | 1077 | 1H NMR (500 MHz, DMSO-d6) Shift 10.97 (s, 1H), 8.67 (d, |
| | [M + H]⁺, | J = 1.9 Hz, 1H), 8.58 (s, 1H), 8.50 (br s, 1H), 8.37 (d, J = 2.3 Hz, |
| | RT = 2.03 | 1H), 7.84 (dd, J = 8.7, 2.3 Hz, 1H), 7.55-7.49 (m, 1H), 7.49-7.36 |
| | min (A) | (m, 3H), 7.32-7.26 (m, 2H), 7.20 (d, J = 1.8 Hz, 1H), 7.09 (d, |
| | | J = 8.8 Hz, 1H), 5.09 (dd, J = 13.2, 5.0 Hz, 1H), 4.41-4.11 (m, 5H), |
| | | 3.31-3.10 (m, 6H), 2.97-2.86 (m, 1H), 2.67-2.57 (m, 1H), 2.49- |
| | | 2.34 (m, 3H), 2.31-2.20 (m, 4H), 2.16-2.04 (m, 3H), 2.04- |
| | | 1.95 (m, 1H), 1.82-1.67 (m, 1H), 1.47-1.27 (m, 3H) 4 |
| | | methylene missing due to water suppression and DMSO |
| | | overlapping |
| 87 | 1051 | NA |
| | [M + H]⁺, | |
| | RT = 1.32 | |
| | min (A) | |
| 88 | 1050 | 1H NMR (500 MHz, DMSO-d6) Shift 8.67-8.60 (m, 1H), 8.59- |
| | [M + H]⁺, | 8.53 (m, 1H), 8.35-8.28 (m, 1H), 8.12-8.00 (m, 1H), 7.83-7.69 |
| | RT = 1.33 | (m, 1H), 7.68-7.62 (m, 1H), 7.51-7.45 (m, 1H), 7.43-7.37 (m, |
| | min (A) | 1H), 7.37-7.28 (m, 3H), 7.28-7.23 (m, 1H), 7.01-6.92 (m, 1H), |
| | | 5.15-5.02 (m, 1H), 4.49-4.36 (m, 1H), 4.35-4.26 (m, 1H), 4.23- |
| | | 4.08 (m, 4H), 3.22-3.09 (m, 2H), 3.07-2.98 (m, 2H), 2.96- |
| | | 2.86 (m, 1H), 2.75-2.57 (m, 6H), 2.45-2.23 (m, 3H), 2.18-2.07 |
| | | (m, 2H), 2.05-1.95 (m, 1H), 1.82-1.54 (m, 6H), 1.49-1.35 (m, |
| | | 8H), 1.35-1.26 (m, 2H) 6 methylene missing due to water |
| | | suppression and DMSO overlapping |
| 89 | 994 | 1H NMR (500 MHz, DMSO-d6) Shift 8.65-8.59 (m, 1H), 8.57- |
| | [M + H]⁺, | 8.52 (m, 1H), 8.35-8.28 (m, 1H), 8.07-7.97 (m, 1H), 7.81-7.70 |
| | RT = 1.27 | (m, 1H), 7.70-7.60 (m, 1H), 7.51-7.44 (m, 1H), 7.42-7.28 (m, |
| | min (A) | 4H), 7.28-7.23 (m, 1H), 7.03-6.84 (m, 1H), 5.18-5.00 (m, 1H), |
| | | 4.53-4.37 (m, 1H), 4.35-4.25 (m, 1H), 4.21-4.06 (m, 5H), 3.94- |
| | | 3.81 (m, 2H), 3.42-3.31 (m, 1H), 3.21-3.08 (m, 2H), 3.01- |
| | | 2.82 (m, 3H), 2.81-2.70 (m, 2H), 2.68-2.56 (m, 2H), 2.45-2.35 |
| | | (m, 1H), 2.35-2.26 (m, 2H), 2.26-2.17 (m, 2H), 2.13-2.04 (m, |
| | | 2H), 2.03-1.95 (m, 1H), 1.82-1.73 (m, 2H), 1.73-1.63 (m, 2H), |
| | | 1.57-1.43 (m, 2H), 1.42-1.33 (m, 3H) 3 methylenes missing due |
| | | to water suppression and DMSO overlapping |
| 90 | 1051 | 1H NMR (500 MHz, DMSO-d6) Shift 8.66-8.59 (m, 1H), 8.58- |
| | [M + H]⁺, | 8.51 (m, 1H), 8.35-8.29 (m, 1H), 8.13-8.05 (m, 1H), 7.81-7.71 |
| | RT = 1.37 | (m, 1H), 7.59-7.46 (m, 1H), 7.39-7.28 (m, 3H), 7.28-7.22 (m, |
| | min (A) | 1H), 7.09-7.00 (m, 2H), 7.00-6.88 (m, 1H), 5.09-4.91 (m, 1H), |
| | | 4.40-4.27 (m, 1H), 4.24-4.04 (m, 5H), 3.39-3.26 (m, 2H), 3.21- |
| | | 3.09 (m, 2H), 2.95-2.82 (m, 1H), 2.75-2.65 (m, 2H), 2.48- |
| | | 2.27 (m, 6H), 2.02-1.94 (m, 1H), 1.94-1.87 (m, 2H), 1.67-1.52 |
| | | (m, 5H), 1.51-1.42 (m, 2H), 1.42-1.32 (m, 5H) 8 methylenes |
| | | missing due to water suppression and DMSO overlapping |
| 91 | 1051 | 1H NMR (500 MHz, DMSO-d6) δ 8.69-8.62 (m, 1H), 8.61-8.54 |
| | [M + H]⁺, | (m, 1H), 8.37-8.28 (m, 1H), 8.17-8.10 (m, 1H), 7.79-7.71 (m, |
| | RT = 1.82 | 1H), 7.55-7.46 (m, 1H), 7.40-7.30 (m, 3H), 7.30-7.24 (m, 1H), |
| | min (A) | 7.07-6.94 (m, 2H), 5.09-4.96 (m, 1H), 4.37-4.26 (m, 1H), 4.25- |
| | | 4.09 (m, 4H), 3.95-3.86 (m, 1H), 3.47-3.36 (m, 1H), 3.33- |
| | | 3.25 (m, 1H), 3.22-3.12 (m, 1H), 2.95-2.83 (m, 1H), 2.78-2.70 |
| | | (m, 2H), 2.66-2.56 (m, 3H), 2.49-2.42 (m, 3H), 2.42-2.28 (m, |
| | | 4H), 2.01-1.94 (m, 1H), 1.94-1.88 (m, 3H), 1.68-1.57 (m, 2H), |
| | | 1.57-1.44 (m, 6H), 1.42-1.36 (m, 3H) 7 methylene missing due |
| | | to water suppression and DMSO overlapping |
| 92 | 1048 | ¹H NMR (500 MHz, DMSO-d₆) δ 8.67-8.61 (m, 1H), 8.59-8.53 |
| | [M + H]⁺, | (m, 1H), 8.35-8.29 (m, 1H), 8.17-8.09 (m, 1H), 7.80-7.72 (m, |
| | RT = 1.88 | 1H), 7.68-7.62 (m, 1H), 7.51-7.45 (m, 1H), 7.42-7.30 (m, 4H), |
| | min (A) | 7.29-7.23 (m, 1H), 7.01-6.93 (m, 1H), 5.18-5.00 (m, 1H), 4.48- |
| | | 4.38 (m, 1H), 4.34-4.25 (m, 1H), 4.23-4.10 (m, 4H), 2.75- |
| | | 2.59 (m, 5H), 2.49-2.29 (m, 11H), 2.29-2.07 (m, 6H), 2.06- |
| | | 1.95 (m, 3H), 1.94-1.89 (m, 2H), 1.89-1.72 (m, 5H), 1.71-1.56 |
| | | (m, 4H), 1.43-1.35 (m, 3H) 2 methylene missing due to water |
| | | suppression and DMSO overlapping. |
| 93 | 980 | 1H NMR (500 MHz, DMSO-d6) Shift 10.54-10.36 (m, 1H), 8.67- |
| | [M + H]⁺, | 8.61 (m, 1H), 8.60-8.54 (m, 1H), 8.35-8.29 (m, 1H), 8.15- |
| | RT = 2.12 | 8.07 (m, 1H), 7.79-7.69 (m, 1H), 7.40-7.30 (m, 3H), 7.30-7.24 |
| | min (A) | (m, 2H), 7.01-6.93 (m, 1H), 6.50-6.40 (m, 1H), 6.22-6.10 (m, |
| | | 2H), 4.25-4.11 (m, 4H), 3.88-3.83 (m, 2H), 3.82-3.72 (m, 3H), |
| | | 3.26-3.11 (m, 2H), 2.77-2.69 (m, 4H), 2.69-2.58 (m, 1H), 2.50- |
| | | 2.43 (m, 4H), 2.43-2.22 (m, 5H), 2.21-2.12 (m, 1H), 2.12- |
| | | 2.02 (m, 1H), 1.95-1.84 (m, 1H), 1.83-1.73 (m, 1H), 1.68-1.56 |
| | | (m, 2H), 1.43-1.33 (m, 3H) 3 methylenes missing due to water |
| | | suppression and DMSO overlapping |
| 94 | 1049 | ¹H NMR (500 MHz, DMSO-d₆) δ 8.64-8.59 (m, 1H), 8.58-8.54 |
| | [M + H]⁺, | (m, 1H), 8.34-8.29 (m, 1H), 8.13-8.06 (m, 1H), 7.78-7.71 (m, |
| | RT = 1.85 | 1H), 7.58-7.50 (m, 1H), 7.37-7.28 (m, 3H), 7.27-7.22 (m, 1H), |
| | min (A) | 7.10-7.03 (m, 2H), 6.99-6.92 (m, 1H), 5.05-4.98 (m, 1H), 4.39- |
| | | 4.31 (m, 1H), 4.25-4.19 (m, 1H), 4.19-4.09 (m, 3H), 3.92- |
| | | 3.86 (m, 1H), 3.68-3.54 (m, 2H), 3.55-3.47 (m, 3H), 3.32-3.22 |
| | | (m, 2H), 3.19-3.10 (m, 3H), 2.95-2.83 (m, 1H), 2.75-2.67 (m, |
| | | 1H), 2.66-2.57 (m, 2H), 2.43-2.25 (m, 5H), 2.23-2.16 (m, 3H), |
| | | 2.16-2.01 (m, 3H), 2.01-1.92 (m, 2H), 1.88-1.78 (m, 1H), 1.76- |
| | | 1.66 (m, 1H), 1.64-1.52 (m, 2H), 1.42-1.34 (m, 3H) 4 |
| | | methylenes missing due to water suppression and DMSO |
| | | overlapping |
| 95 | 1048 | ¹H NMR (500 MHz, DMSO-d₆) δ 8.65-8.58 (m, 1H), 8.58-8.52 |
| | [M + H]⁺, | (m, 1H), 8.35-8.27 (m, 1H), 8.12-8.04 (m, 1H), 7.81-7.70 (m, |
| | RT = 1.89 | 1H), 7.70-7.60 (m, 1H), 7.52-7.45 (m, 1H), 7.43-7.37 (m, 1H), |
| | min (A) | 7.36-7.28 (m, 3H), 7.27-7.22 (m, 1H), 7.00-6.91 (m, 1H), 5.13- |
| | | 4.99 (m, 1H), 4.54-4.46 (m, 1H), 4.45-4.37 (m, 1H), 4.34- |
| | | 4.25 (m, 1H), 4.21-4.08 (m, 3H), 3.92-3.87 (m, 1H), 3.84-3.55 |
| | | (m, 5H), 3.56-3.47 (m, 5H), 3.20-3.12 (m, 2H), 3.08-2.97 (m, |
| | | 1H), 2.94-2.83 (m, 2H), 2.74-2.57 (m, 4H), 2.44-2.34 (m, 2H), |
| | | 2.34-2.25 (m, 3H), 2.25-2.13 (m, 4H), 2.08-1.93 (m, 3H), 1.86- |
| | | 1.75 (m, 3H), 1.76-1.66 (m, 1H), 1.63-1.53 (m, 2H), 1.54- |
| | | 1.42 (m, 2H), 1.42-1.33 (m, 3H) |
| 96 | 939 | 1H NMR (500 MHz, DMSO-d6) Shift 11.06-10.89 (m, 1H), 8.65- |
| | [M + H]⁺, | 8.58 (m, 1H), 8.58-8.51 (m, 1H), 8.39-8.28 (m, 1H), 8.19- |
| | RT = 1.92 | 7.94 (m, 1H), 7.80-7.72 (m, 1H), 7.72-7.59 (m, 3H), 7.42-7.28 |
| | min (A) | (m, 3H), 7.28-7.20 (m, 1H), 7.04-6.90 (m, 1H), 5.22-4.99 (m, |
| | | 1H), 4.60-4.45 (m, 1H), 4.44-4.30 (m, 1H), 4.24-4.07 (m, 4H), |
| | | 3.22-3.07 (m, 2H), 2.97-2.84 (m, 1H), 2.68-2.57 (m, 1H), 2.47- |
| | | 2.25 (m, 3H), 2.09-1.96 (m, 1H), 1.70-1.44 (m, 5H), 1.69- |
| | | 1.42 (m, 6H), 1.41-1.34 (m, 4H) 4 methylenes missing due to |
| | | water suppression and DMSO overlapping |
| 97 | 913 | 1H NMR (500 MHz, DMSO-d6) Shift 11.06-10.82 (m, 1H), 8.65- |
| | [M + H]⁺, | 8.61 (m, 1H), 8.58-8.54 (m, 1H), 8.36-8.27 (m, 1H), 8.08- |
| | RT = 1.24 | 7.95 (m, 1H), 7.81-7.71 (m, 1H), 7.67-7.59 (m, 1H), 7.40-7.29 |
| | min (A) | (m, 3H), 7.29-7.23 (m, 1H), 7.16-7.11 (m, 1H), 7.08-7.02 (m, |
| | | 1H), 7.00-6.92 (m, 1H), 5.11-5.01 (m, 1H), 4.73-4.61 (m, 2H), |
| | | 4.44-4.34 (m, 1H), 4.34-4.22 (m, 3H), 4.20-4.09 (m, 4H), 4.03- |
| | | 3.93 (m, 2H), 3.22-3.09 (m, 1H), 2.96-2.84 (m, 1H), 2.82- |
| | | 2.73 (m, 1H), 2.66-2.57 (m, 1H), 2.46-2.25 (m, 3H), 2.05-1.94 |
| | | (m, 1H), 1.57-1.45 (m, 2H), 1.42-1.34 (m, 3H) 2 methylene |
| | | missing due to water suppression and DMSO overlapping |
| 98 | 953 | 1H NMR (500 MHz, DMSO-d6) Shift 11.02-10.95 (m, 1H), 8.68- |
| | [M + H]⁺, | 8.63 (m, 1H), 8.59-8.55 (m, 1H), 8.53-8.48 (m, 1H), 8.38- |
| | RT = 1.93 | 8.33 (m, 1H), 7.87-7.80 (m, 1H), 7.72-7.62 (m, 1H), 7.54-7.48 |
| | min (A) | (m, 1H), 7.48-7.34 (m, 4H), 7.30-7.25 (m, 1H), 7.11-7.04 (m, |
| | | 1H), 5.19-5.01 (m, 1H), 4.51-4.38 (m, 1H), 4.35-4.21 (m, 3H), |
| | | 4.20-4.10 (m, 2H), 3.93-3.83 (m, 2H), 3.71-3.60 (m, 1H), 3.27- |
| | | 3.15 (m, 2H), 2.98-2.84 (m, 1H), 2.66-2.57 (m, 1H), 2.48- |
| | | 2.30 (m, 3H), 2.07-1.96 (m, 1H), 1.86-1.62 (m, 5H), 1.60-1.51 |
| | | (m, 2H), 1.42-1.35 (m, 3H), 1.35-1.26 (m, 2H) 5 Methylene |
| | | missing due to water suppression and DMSO overlapping |
| 99 | 970 | 1H NMR (500 MHz, DMSO-d6) Shift 8.65-8.60 (m, 1H), 8.59- |
| | [M + H]⁺, | 8.53 (m, 1H), 8.34-8.29 (m, 1H), 8.12-8.02 (m, 1H), 7.80-7.71 |
| | RT = 1.94 | (m, 1H), 7.66-7.59 (m, 1H), 7.40-7.29 (m, 3H), 7.29-7.21 (m, |
| | min (A) | 1H), 7.16-7.09 (m, 1H), 7.08-7.01 (m, 1H), 7.00-6.90 (m, 1H), |
| | | 5.11-5.01 (m, 1H), 4.95-4.87 (m, 2H), 4.44-4.34 (m, 1H), 4.30- |
| | | 4.23 (m, 1H), 4.21-4.10 (m, 4H), 3.20-3.11 (m, 2H), 2.96- |
| | | 2.84 (m, 1H), 2.74-2.67 (m, 1H), 2.64-2.57 (m, 1H), 2.42-2.26 |
| | | (m, 3H), 2.05-1.94 (m, 1H), 1.68-1.55 (m, 2H), 1.52-1.42 (m, |
| | | 5H), 1.41-1.28 (m, 5H) 5 methylene missing due to water |
| | | suppression and DMSO overlapping |

Examples 100-135 were prepared using procedures similar to the ones described in the aforementioned examples.

### Example 100

### N-[1-(5-{3-cyano-6-ethoxypyrazolo[1,5-a]pyridin-4-yl}pyridin-2-yl)-4-({4-[2-(3-{4-[2-(2,6-dioxopiperidin-3-yl)-1-oxo-2,3-dihydro-1H-isoindol-5-yl]piperazin-1-yl}-3-oxopropoxy)ethyl]piperazin-1-yl}methyl)piperidin-4-yl]-2,5-difluorobenzamide

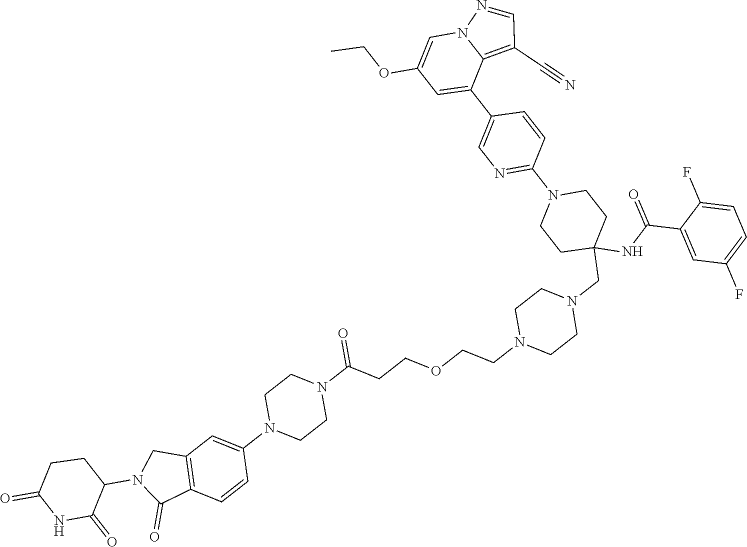 [SMILES: CCOc1cc(-c2ccc(N3CCC(CN4CCN(CCOCCC(=O)N5CCN(c6ccc7c(c6)CN(C6CCC(=O)NC6=O)C7=O)CC5)CC4)(NC(=O)c4cc(F)ccc4F)CC3)nc2)c2c(C#N)cnn2c1]

### Example 101

### N-[1-(5-{3-cyano-6-ethoxypyrazolo[1,5-a]pyridin-4-yl}pyridin-2-yl)-4-({4-[2-(3-{4-[2-(2,6-dioxopiperidin-3-yl)-3-oxo-2,3-dihydro-1H-isoindol-5-yl]piperidin-1-yl}-3-oxopropoxy)ethyl]piperazin-1-yl}methyl)piperidin-4-yl]-2,5-difluorobenzamide

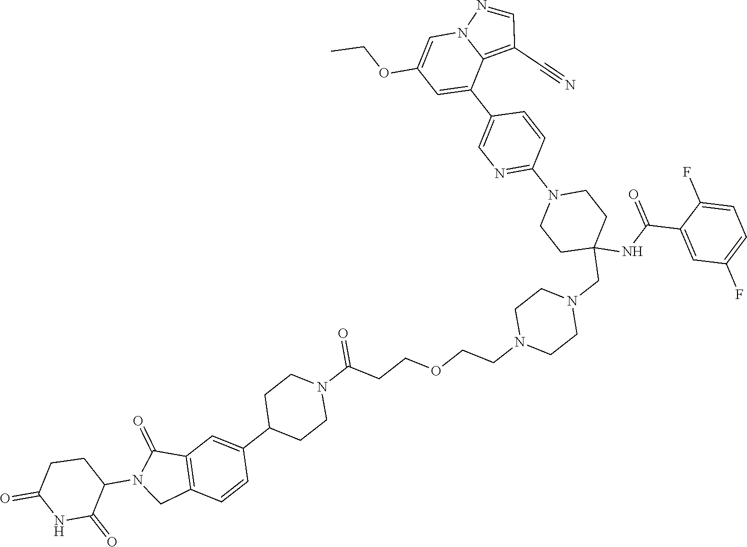 [SMILES: CCOc1cc(-c2ccc(N3CCC(CN4CCN(CCOCCC(=O)N5CCC(c6ccc7c(c6)C(=O)N(C6CCC(=O)NC6=O)C7)CC5)CC4)(NC(=O)c4cc(F)ccc4F)CC3)nc2)c2c(C#N)cnn2c1]

### Example 102

### N-[1-(5-{3-cyano-6-ethoxypyrazolo[1,5-a]pyridin-4-yl}pyridin-2-yl)-4-({4-[2-(2-{[3-(2,4-dioxo-1,3-diazinan-1-yl)-1-methyl-1H-indazol-6-yl]carbamoyl}ethoxy)ethyl]piperazin-1-yl}methyl)piperidin-4-yl]-2,5-difluorobenzamide

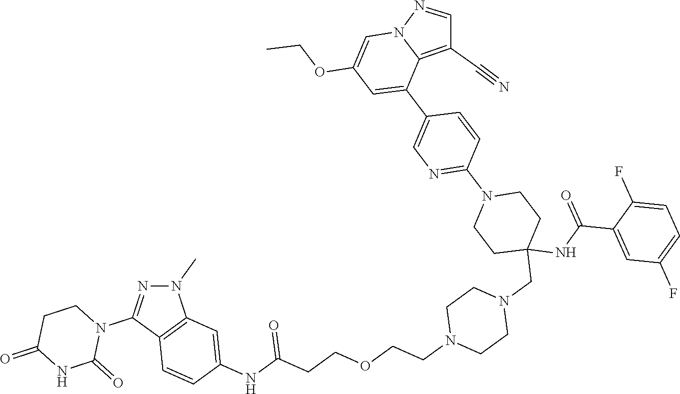 [SMILES: CCOc1cc(-c2ccc(N3CCC(CN4CCN(CCOCCC(=O)Nc5ccc6c(N7CCC(=O)NC7=O)nn(C)c6c5)CC4)(NC(=O)c4cc(F)ccc4F)CC3)nc2)c2c(C#N)cnn2c1]

### Example 103

### N-[1-(5-{3-cyano-6-ethoxypyrazolo[1,5-a]pyridin-4-yl}pyridin-2-yl)-4-({4-[2-(3-{4-[2-(2,6-dioxopiperidin-3-yl)-3-oxo-2,3-dihydro-1H-isoindol-5-yl]piperazin-1-yl}-3-oxopropoxy)ethyl]piperazin-1-yl}methyl)piperidin-4-yl]-2,5-difluorobenzamide

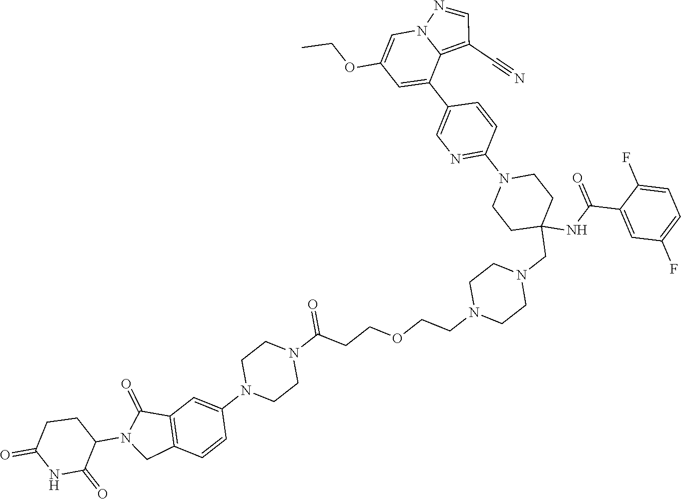 [SMILES: CCOc1cc(-c2ccc(N3CCC(CN4CCN(CCOCCC(=O)N5CCN(c6ccc7c(c6)C(=O)N(C6CCC(=O)NC6=O)C7)CC5)CC4)(NC(=O)c4cc(F)ccc4F)CC3)nc2)c2c(C#N)cnn2c1]

### Example 104

### N-[1-(5-{3-cyano-6-ethoxypyrazolo[1,5-a]pyridin-4-yl}pyridin-2-yl)-4-({4-[3-(2-{4-[2-(2,6-dioxopiperidin-3-yl)-1-oxo-2,3-dihydro-1H-isoindol-5-yl]piperazin-1-yl}ethoxy)propanoyl]piperazin-1-yl}methyl)piperidin-4-yl]-2,5-difluorobenzamide

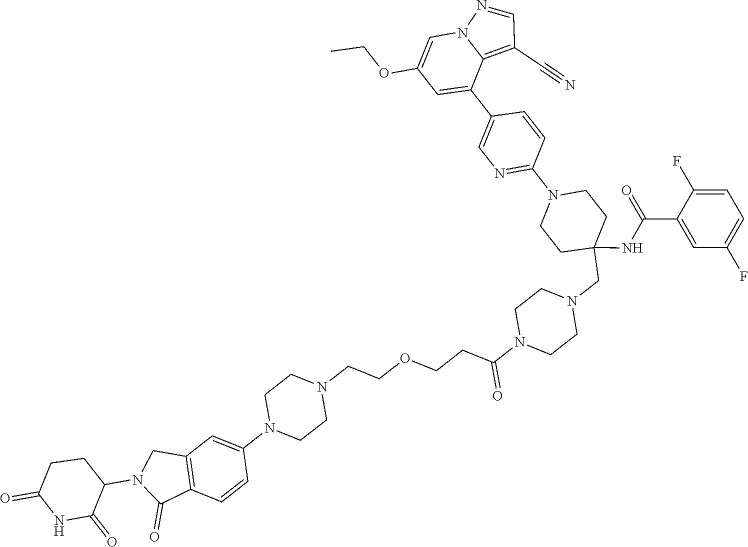 [SMILES: CCOc1cc(-c2ccc(N3CCC(CN4CCN(C(=O)CCOCCN5CCN(c6ccc7c(c6)CN(C6CCC(=O)NC6=O)C7=O)CC5)CC4)(NC(=O)c4cc(F)ccc4F)CC3)nc2)c2c(C#N)cnn2c1]

### Example 105

### N-[1-(5-{3-cyano-6-ethoxypyrazolo[1,5-a]pyridin-4-yl}pyridin-2-yl)-4-({4-[3-(2-{[3-(2,4-dioxo-1,3-diazinan-1-yl)-1-methyl-1H-indazol-6-yl]amino}ethoxy)propanoyl]piperazin-1-yl}methyl)piperidin-4-yl]-2,5-difluorobenzamide

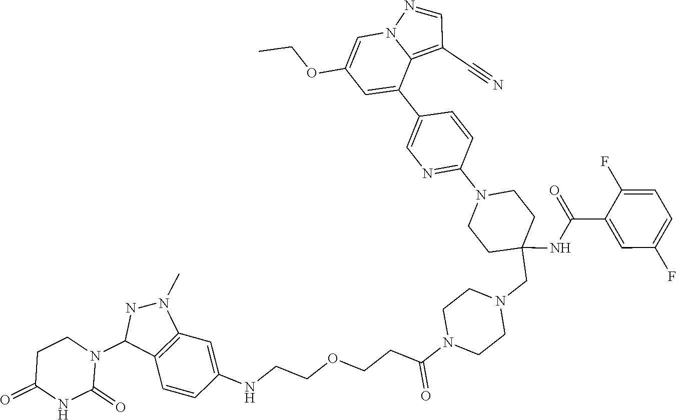 [SMILES: CCOc1cc(-c2ccc(N3CCC(CN4CCN(C(=O)CCOCCNc5ccc6c(c5)N(C)NC6N5CCC(=O)NC5=O)CC4)(NC(=O)c4cc(F)ccc4F)CC3)nc2)c2c(C#N)cnn2c1]

### Example 106

### N-[1-(5-{3-cyano-6-ethoxypyrazolo[1,5-a]pyridin-4-yl}pyridin-2-yl)-4-[(4-{2-[2-(2,6-dioxopiperidin-3-yl)-3-oxo-2,3-dihydro-1H-isoindol-5-yl]acetyl}piperazin-1-yl)methyl]piperidin-4-yl]-2,5-difluorobenzamide

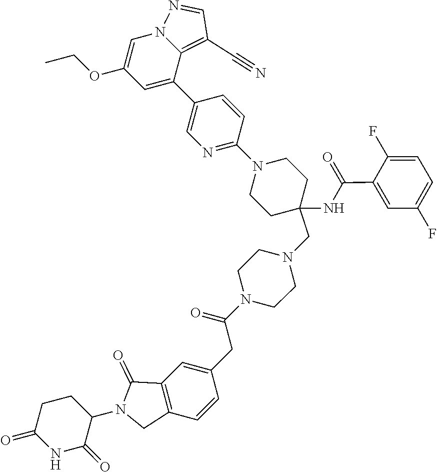 [SMILES: CCOc1cc(-c2ccc(N3CCC(CN4CCN(C(=O)Cc5ccc6c(c5)C(=O)N(C5CCC(=O)NC5=O)C6)CC4)(NC(=O)c4cc(F)ccc4F)CC3)nc2)c2c(C#N)cnn2c1]

### Example 107

### N-[1-(5-{3-cyano-6-ethoxypyrazolo[1,5-a]pyridin-4-yl}pyridin-2-yl)-4-{[4-(2-{[2-(2,6-dioxopiperidin-3-yl)-1-oxo-2,3-dihydro-1H-isoindol-5-yl]oxy}acetyl)piperazin-1-yl]methyl}piperidin-4-yl]-2,5-difluorobenzamide

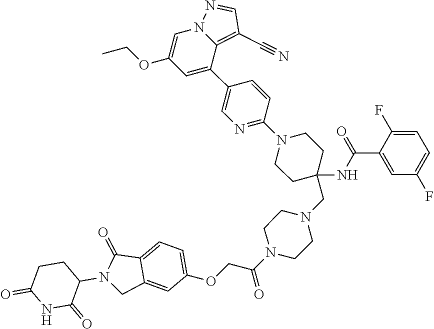 [SMILES: CCOc1cc(-c2ccc(N3CCC(CN4CCN(C(=O)COc5ccc6c(c5)CN(C5CCC(=O)NC5=O)C6=O)CC4)(NC(=O)c4cc(F)ccc4F)CC3)nc2)c2c(C#N)cnn2c1]

### Example 108

### N-[1-(5-{3-cyano-6-ethoxypyrazolo[1,5-a]pyridin-4-yl}pyridin-2-yl)-4-[(4-{2-[2-(2-{[3-(2,4-dioxo-1,3-diazinan-1-yl)-1-methyl-1H-indazol-6-yl]carbamoyl}ethoxy)ethoxy]ethyl}piperazin-1-yl)methyl]piperidin-4-yl]-2,5-difluorobenzamide

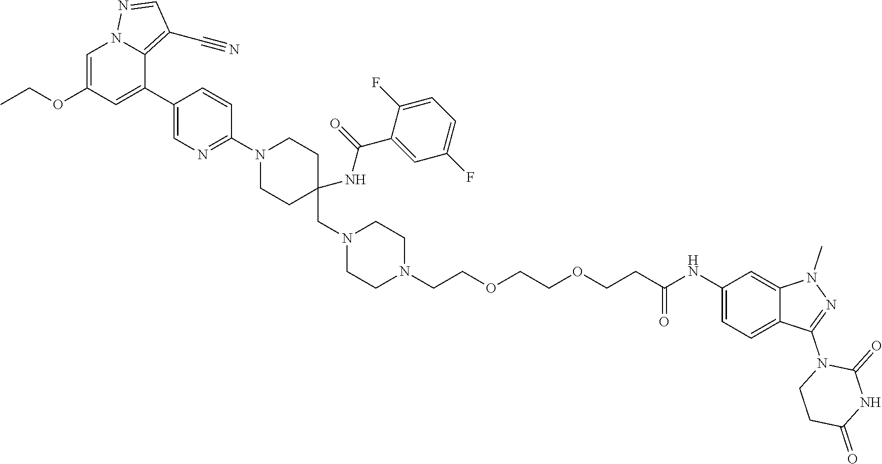 [SMILES: CCOc1cc(-c2ccc(N3CCC(CN4CCN(CCOCCOCCC(=O)Nc5ccc6c(N7CCC(=O)NC7=O)nn(C)c6c5)CC4)(NC(=O)c4cc(F)ccc4F)CC3)nc2)c2c(C#N)cnn2c1]

### Example 109

### N-[1-(5-{3-cyano-6-ethoxypyrazolo[1,5-a]pyridin-4-yl}pyridin-2-yl)-4-[(4-{2-[2-(3-{4-[2-(2,6-dioxopiperidin-3-yl)-1-oxo-2,3-dihydro-1H-isoindol-5-yl]piperazin-1-yl}-3-oxopropoxy)ethoxy]ethyl}piperazin-1-yl)methyl]piperidin-4-yl]-2,5-difluorobenzamide

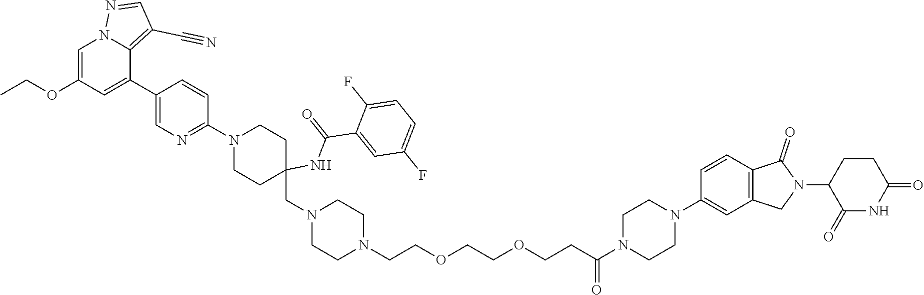 [SMILES: CCOc1cc(-c2ccc(N3CCC(CN4CCN(CCOCCOCCC(=O)N5CCN(c6ccc7c(c6)CN(C6CCC(=O)NC6=O)C7=O)CC5)CC4)(NC(=O)c4cc(F)ccc4F)CC3)nc2)c2c(C#N)cnn2c1]

### Example 110

### N-[1-(5-{3-cyano-6-ethoxypyrazolo[1,5-a]pyridin-4-yl}pyridin-2-yl)-4-{[4-({1-[2-(2,6-dioxopiperidin-3-yl)-3-oxo-2,3-dihydro-1H-isoindole-5-carbonyl]piperidin-4-yl}methyl)piperazin-1-yl]methyl}piperidin-4-yl]-2,5-difluorobenzamide

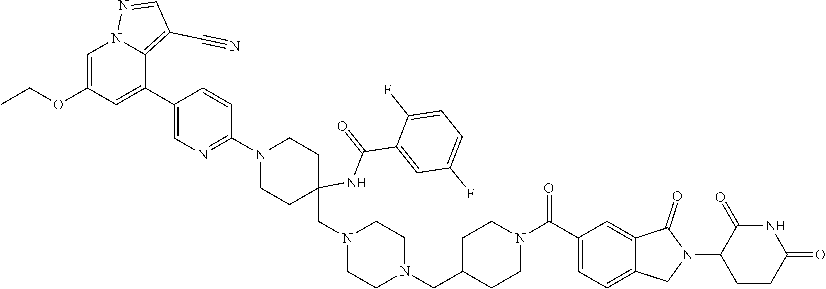 [SMILES: CCOc1cc(-c2ccc(N3CCC(CN4CCN(CC5CCN(C(=O)c6ccc7c(c6)C(=O)N(C6CCC(=O)NC6=O)C7)CC5)CC4)(NC(=O)c4cc(F)ccc4F)CC3)nc2)c2c(C#N)cnn2c1]

### Example 111

### N-[1-(5-{3-cyano-6-ethoxypyrazolo[1,5-a]pyridin-4-yl}pyridin-2-yl)-4-({4-[(1-{2-[2-(2,6-dioxopiperidin-3-yl)-1-oxo-2,3-dihydro-1H-isoindol-4-yl]acetyl}piperidin-4-yl)methyl]piperazin-1-yl}methyl)piperidin-4-yl]-2,5-difluorobenzamide

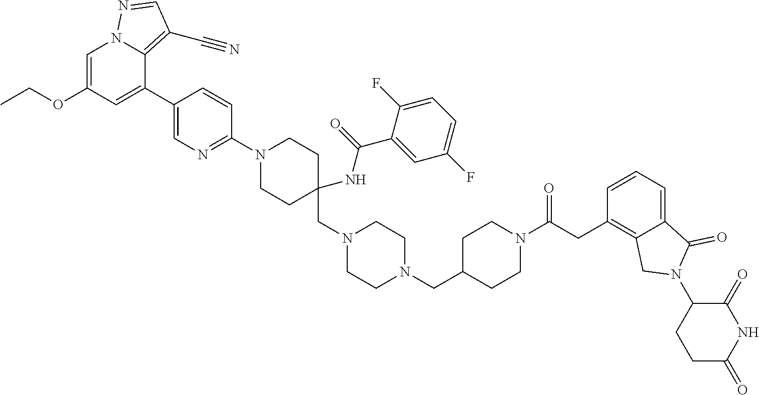 [SMILES: CCOc1cc(-c2ccc(N3CCC(CN4CCN(CC5CCN(C(=O)Cc6cccc7c6CN(C6CCC(=O)NC6=O)C7=O)CC5)CC4)(NC(=O)c4cc(F)ccc4F)CC3)nc2)c2c(C#N)cnn2c1]

### Example 112

### N-[1-(5-{3-cyano-6-ethoxypyrazolo[1,5-a]pyridin-4-yl}pyridin-2-yl)-4-({4-[(1-{2-[2-(2,6-dioxopiperidin-3-yl)-1-oxo-2,3-dihydro-1H-isoindol-5-yl]acetyl}piperidin-4-yl)methyl]piperazin-1-yl}methyl)piperidin-4-yl]-2,5-difluorobenzamide

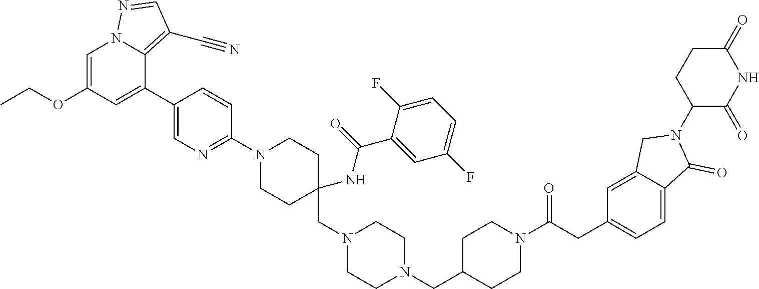 [SMILES: CCOc1cc(-c2ccc(N3CCC(CN4CCN(CC5CCN(C(=O)Cc6ccc7c(c6)CN(C6CCC(=O)NC6=O)C7=O)CC5)CC4)(NC(=O)c4cc(F)ccc4F)CC3)nc2)c2c(C#N)cnn2c1]

### Example 113

### N-[1-(5-{3-cyano-6-ethoxypyrazolo[1,5-a]pyridin-4-yl}pyridin-2-yl)-4-({4-[(1-{2-[2-(2,6-dioxopiperidin-3-yl)-3-oxo-2,3-dihydro-1H-isoindol-5-yl]acetyl}piperidin-4-yl)methyl]piperazin-1-yl}methyl)piperidin-4-yl]-2,5-difluorobenzamide

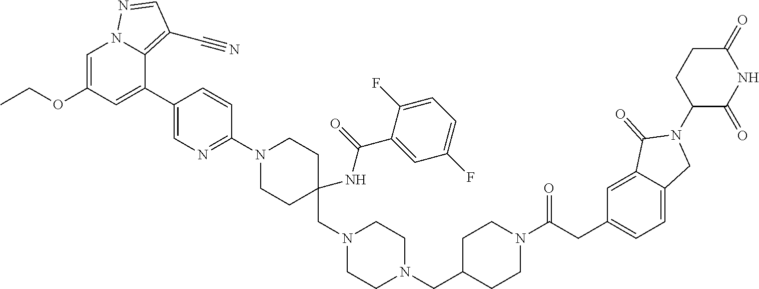 [SMILES: CCOc1cc(-c2ccc(N3CCC(CN4CCN(CC5CCN(C(=O)Cc6ccc7c(c6)C(=O)N(C6CCC(=O)NC6=O)C7)CC5)CC4)(NC(=O)c4cc(F)ccc4F)CC3)nc2)c2c(C#N)cnn2c1]

### Example 114

### N-[1-(5-{3-cyano-6-ethoxypyrazolo[1,5-a]pyridin-4-yl}pyridin-2-yl)-4-[(4-{[1-(2-{[2-(2,6-dioxopiperidin-3-yl)-1-oxo-2,3-dihydro-1H-isoindol-5-yl]oxy}acetyl)piperidin-4-yl]methyl}piperazin-1-yl)methyl]piperidin-4-yl]-2,5-difluorobenzamide

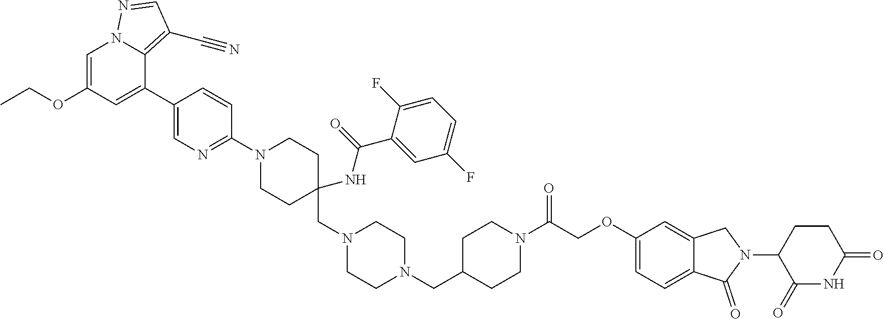 [SMILES: CCOc1cc(-c2ccc(N3CCC(CN4CCN(CC5CCN(C(=O)COc6ccc7c(c6)CN(C6CCC(=O)NC6=O)C7=O)CC5)CC4)(NC(=O)c4cc(F)ccc4F)CC3)nc2)c2c(C#N)cnn2c1]

### Example 115

### N-[1-(5-{3-cyano-6-ethoxypyrazolo[1,5-a]pyridin-4-yl}pyridin-2-yl)-4-{[4-(3-{4-[2-(2,6-dioxopiperidin-3-yl)-3-oxo-2,3-dihydro-1H-isoindol-5-yl]piperazin-1-yl}-3-oxopropyl)piperazin-1-yl]methyl}piperidin-4-yl]-2,5-difluorobenzamide

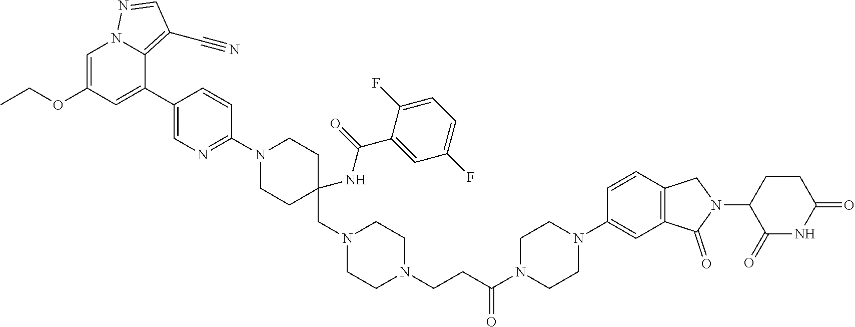 [SMILES: CCOc1cc(-c2ccc(N3CCC(CN4CCN(CCC(=O)N5CCN(c6ccc7c(c6)C(=O)N(C6CCC(=O)NC6=O)C7)CC5)CC4)(NC(=O)c4cc(F)ccc4F)CC3)nc2)c2c(C#N)cnn2c1]

### Example 116

### N-[1-(5-{3-cyano-6-ethoxypyrazolo[1,5-a]pyridin-4-yl}pyridin-2-yl)-4-{[4-(3-{4-[2-(2,6-dioxopiperidin-3-yl)-3-oxo-2,3-dihydro-1H-isoindol-5-yl]piperidin-1-yl}-3-oxopropyl)piperazin-1-yl]methyl}piperidin-4-yl]-2,5-difluorobenzamide

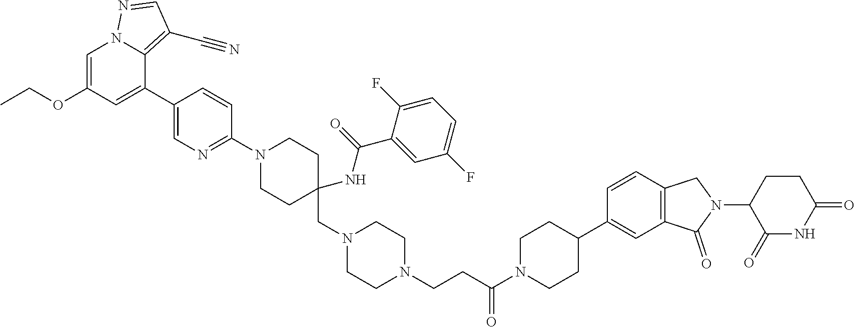 [SMILES: CCOc1cc(-c2ccc(N3CCC(CN4CCN(CCC(=O)N5CCC(c6ccc7c(c6)C(=O)N(C6CCC(=O)NC6=O)C7)CC5)CC4)(NC(=O)c4cc(F)ccc4F)CC3)nc2)c2c(C#N)cnn2c1]

### Example 117

### N-[1-(5-{3-cyano-6-ethoxypyrazolo[1,5-a]pyridin-4-yl}pyridin-2-yl)-4-{[4-(3-{4-[2-(2,6-dioxopiperidin-3-yl)-1,3-dioxo-2,3-dihydro-1H-isoindol-5-yl]piperidin-1-yl}-3-oxopropyl)piperazin-1-yl]methyl}piperidin-4-yl]-2,5-difluorobenzamide

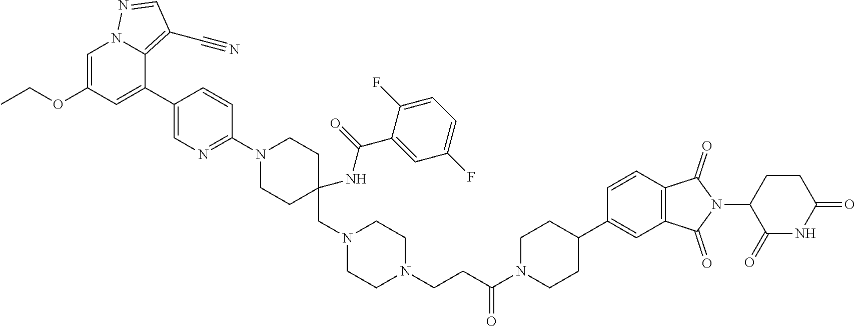 [SMILES: CCOc1cc(-c2ccc(N3CCC(CN4CCN(CCC(=O)N5CCC(c6ccc7c(c6)C(=O)N(C6CCC(=O)NC6=O)C7=O)CC5)CC4)(NC(=O)c4cc(F)ccc4F)CC3)nc2)c2c(C#N)cnn2c1]

### Example 118

### N-[1-(5-{3-cyano-6-ethoxypyrazolo[1,5-a]pyridin-4-yl}pyridin-2-yl)-4-({4-[3-(4-{[2-(2,6-dioxopiperidin-3-yl)-1-oxo-2,3-dihydro-1H-isoindol-5-yl]oxy}piperidin-1-yl)propanoyl]piperazin-1-yl}methyl)piperidin-4-yl]-2,5-difluorobenzamide

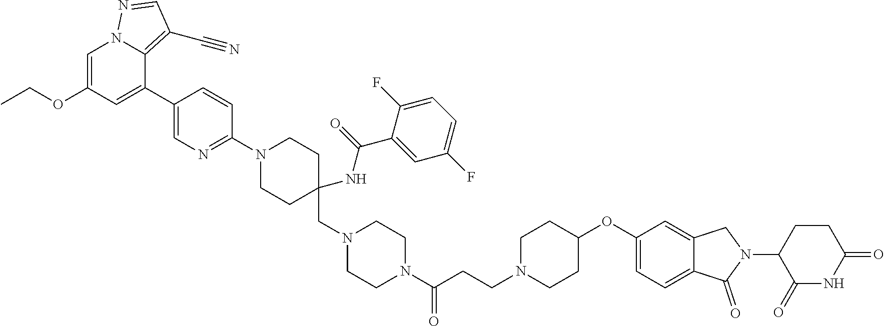 [SMILES: CCOc1cc(-c2ccc(N3CCC(CN4CCN(C(=O)CCN5CCC(Oc6ccc7c(c6)CN(C6CCC(=O)NC6=O)C7=O)CC5)CC4)(NC(=O)c4cc(F)ccc4F)CC3)nc2)c2c(C#N)cnn2c1]

### Example 119

### N-[1-(5-{3-cyano-6-ethoxypyrazolo[1,5-a]pyridin-4-yl}pyridin-2-yl)-4-{[4-(3-{3-[2-(2,6-dioxopiperidin-3-yl)-1-oxo-2,3-dihydro-1H-isoindol-5-yl]azetidin-1-yl}propanoyl)piperazin-1-yl]methyl}piperidin-4-yl]-2,5-difluorobenzamide

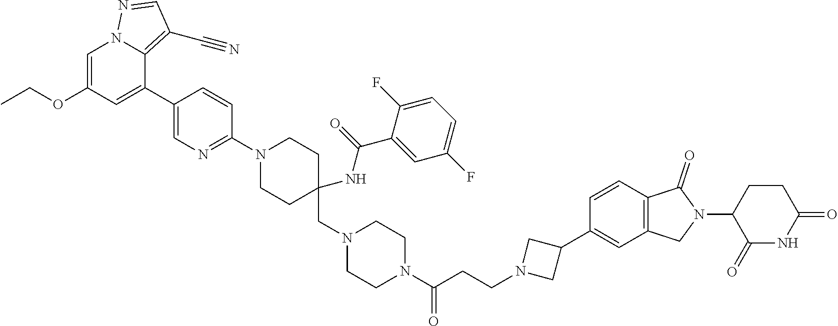 [SMILES: CCOc1cc(-c2ccc(N3CCC(CN4CCN(C(=O)CCN5CC(c6ccc7c(c6)CN(C6CCC(=O)NC6=O)C7=O)C5)CC4)(NC(=O)c4cc(F)ccc4F)CC3)nc2)c2c(C#N)cnn2c1]

### Example 120

### N-[1-(5-{3-cyano-6-ethoxypyrazolo[1,5-a]pyridin-4-yl}pyridin-2-yl)-4-({4-[3-(3-{[2-(2,6-dioxopiperidin-3-yl)-1-oxo-2,3-dihydro-1H-isoindol-5-yl]oxy}azetidin-1-yl)propanoyl]piperazin-1-yl}methyl)piperidin-4-yl]-2,5-difluorobenzamide

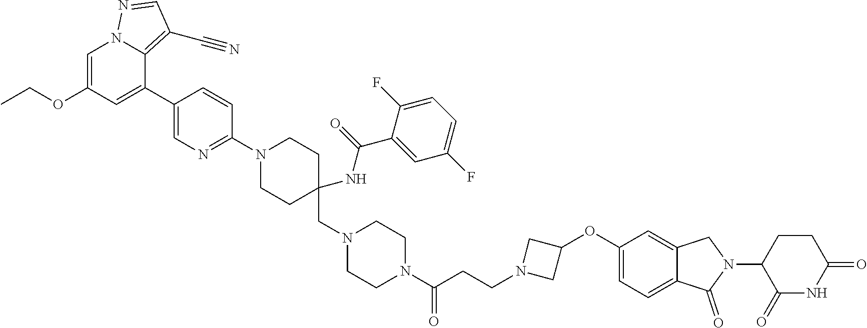 [SMILES: CCOc1cc(-c2ccc(N3CCC(CN4CCN(C(=O)CCN5CC(Oc6ccc7c(c6)CN(C6CCC(=O)NC6=O)C7=O)C5)CC4)(NC(=O)c4cc(F)ccc4F)CC3)nc2)c2c(C#N)cnn2c1]

### Example 121

### N-[1-(5-{3-cyano-6-ethoxypyrazolo[1,5-a]pyridin-4-yl}pyridin-2-yl)-4-{[9-(2-{4-[2-(2,6-dioxopiperidin-3-yl)-3-oxo-2,3-dihydro-1H-isoindol-5-yl]piperidin-1-yl}-2-oxoethyl)-3-azaspiro[5.5]undecan-3-yl]methyl}piperidin-4-yl]-2,5-difluorobenzamide

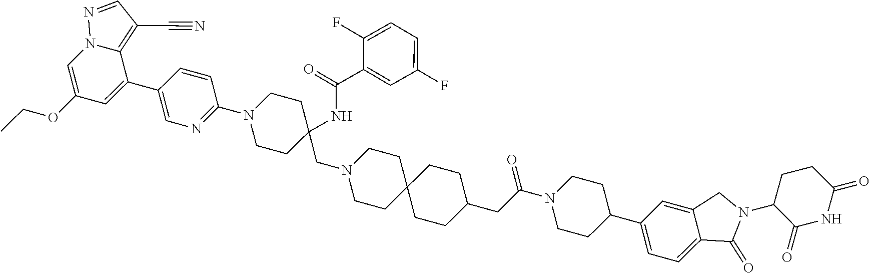 [SMILES: CCOc1cc(-c2ccc(N3CCC(CN4CCC5(CCC(CC(=O)N6CCC(c7ccc8c(c7)CN(C7CCC(=O)NC7=O)C8=O)CC6)CC5)CC4)(NC(=O)c4cc(F)ccc4F)CC3)nc2)c2c(C#N)cnn2c1]

### Example 122

### N-[1-(5-{3-cyano-6-ethoxypyrazolo[1,5-a]pyridin-4-yl}pyridin-2-yl)-4-{[9-(2-{4-[2-(2,6-dioxopiperidin-3-yl)-1-oxo-2,3-dihydro-1H-isoindol-5-yl]piperidin-1-yl}-2-oxoethyl)-3-azaspiro[5.5]undecan-3-yl]methyl}piperidin-4-yl]-2,5-difluorobenzamide

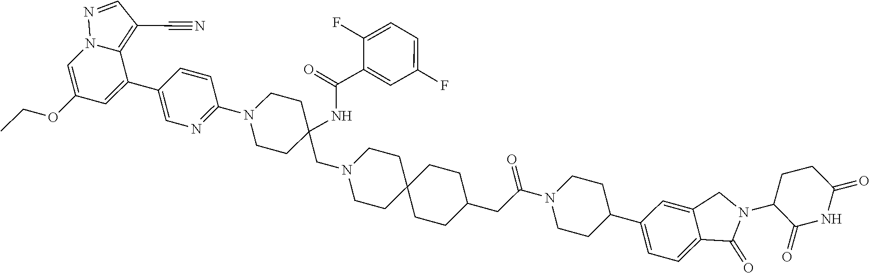 [SMILES: CCOc1cc(-c2ccc(N3CCC(CN4CCC5(CCC(CC(=O)N6CCC(c7ccc8c(c7)CN(C7CCC(=O)NC7=O)C8=O)CC6)CC5)CC4)(NC(=O)c4cc(F)ccc4F)CC3)nc2)c2c(C#N)cnn2c1]

### Example 123

### N-[1-(5-{3-cyano-6-ethoxypyrazolo[1,5-a]pyridin-4-yl}pyridin-2-yl)-4-{[9-(2-{4-[2-(2,6-dioxopiperidin-3-yl)-3-oxo-2,3-dihydro-1H-isoindol-5-yl]piperazin-1-yl}-2-oxoethyl)-3-azaspiro[5.5]undecan-3-yl]methyl}piperidin-4-yl]-2,5-difluorobenzamide

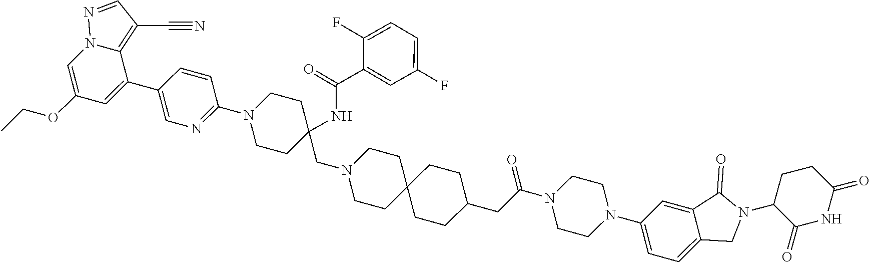 [SMILES: CCOc1cc(-c2ccc(N3CCC(CN4CCC5(CCC(CC(=O)N6CCN(c7ccc8c(c7)C(=O)N(C7CCC(=O)NC7=O)C8)CC6)CC5)CC4)(NC(=O)c4cc(F)ccc4F)CC3)nc2)c2c(C#N)cnn2c1]

### Example 124

### N-[1-(5-{3-cyano-6-ethoxypyrazolo[1,5-a]pyridin-4-yl}pyridin-2-yl)-4-{[9-(2-{4-[2-(2,6-dioxopiperidin-3-yl)-1-oxo-2,3-dihydro-1H-isoindol-5-yl]piperazin-1-yl}-2-oxoethyl)-3-azaspiro[5.5]undecan-3-yl]methyl}piperidin-4-yl]-2,5-difluorobenzamide

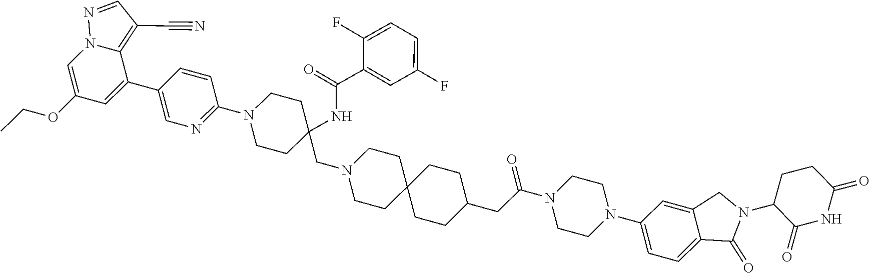 [SMILES: CCOc1cc(-c2ccc(N3CCC(CN4CCC5(CCC(CC(=O)N6CCN(c7ccc8c(c7)CN(C7CCC(=O)NC7=O)C8=O)CC6)CC5)CC4)(NC(=O)c4cc(F)ccc4F)CC3)nc2)c2c(C#N)cnn2c1]

### Example 125

### 4-[6-(4-{[4-(7-{4-[2-(2,6-dioxopiperidin-3-yl)-1-oxo-2,3-dihydro-1H-isoindol-5-yl]piperazin-1-yl}heptanoyl)piperazin-1-yl]methyl}piperidin-1-yl)pyridin-3-yl]-6-ethoxypyrazolo[1,5-a]pyridine-3-carbonitrile

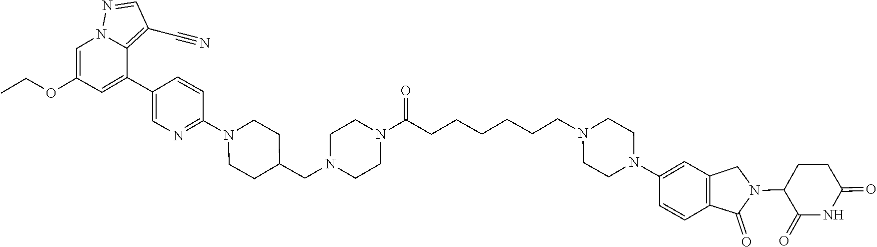 [SMILES: CCOc1cc(-c2ccc(N3CCC(CN4CCN(C(=O)CCCCCCN5CCN(c6ccc7c(c6)CN(C6CCC(=O)NC6=O)C7=O)CC5)CC4)CC3)nc2)c2c(C#N)cnn2c1]

### Example 126

### 4-[6-(4-{[4-(3-{4-[2-(2,6-dioxopiperidin-3-yl)-3-oxo-2,3-dihydro-1H-isoindol-5-yl]piperidin-1-yl}propanoyl)piperazin-1-yl]methyl}piperidin-1-yl)pyridin-3-yl]-6-ethoxypyrazolo[1,5-a]pyridine-3-carbonitrile

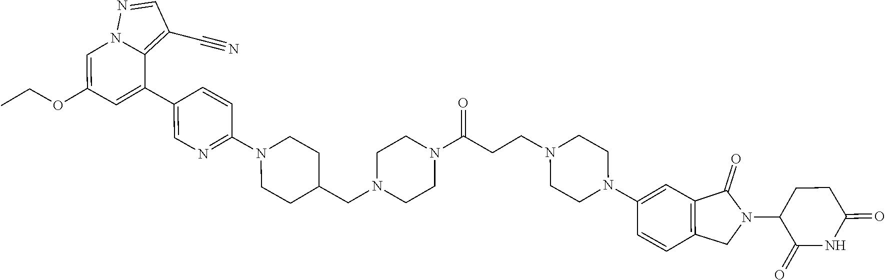 [SMILES: CCOc1cc(-c2ccc(N3CCC(CN4CCN(C(=O)CCN5CCN(c6ccc7c(c6)C(=O)N(C6CCC(=O)NC6=O)C7)CC5)CC4)CC3)nc2)c2c(C#N)cnn2c1]

### Example 127

### N-[1-(5-{3-cyano-6-ethoxypyrazolo[1,5-a]pyridin-4-yl}pyridin-2-yl)-4-[(methylamino)methyl]piperidin-4-yl]-2,5-difluorobenzamide

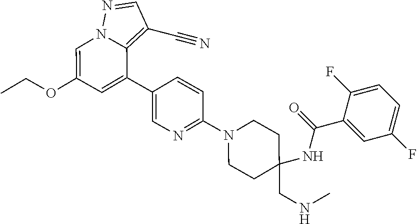 [SMILES: CCOc1cc(-c2ccc(N3CCC(CNC)(NC(=O)c4cc(F)ccc4F)CC3)nc2)c2c(C#N)cnn2c1]

### Example 128

### 4-[6-(4-{[4-(7-{4-[2-(2,6-dioxopiperidin-3-yl)-1-oxo-2,3-dihydro-1H-isoindol-5-yl]piperazin-1-yl}heptanoyl)piperazin-1-yl]methyl}-4-methylpiperidin-1-yl)pyridin-3-yl]-6-ethoxypyrazolo[1,5-a]pyridine-3-carbonitrile

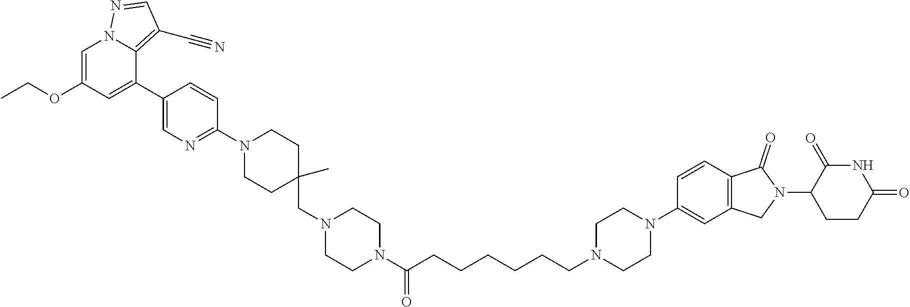 [SMILES: CCOc1cc(-c2ccc(N3CCC(C)(CN4CCN(C(=O)CCCCCCN5CCN(c6ccc7c(c6)CN(C6CCC(=O)NC6=O)C7=O)CC5)CC4)CC3)nc2)c2c(C#N)cnn2c1]

### Example 129

### 4-[6-(4-{[4-(3-{4-[2-(2,6-dioxopiperidin-3-yl)-1-oxo-2,3-dihydro-1H-isoindol-5-yl]piperidin-1-yl}propanoyl)piperazin-1-yl]methyl}-4-methylpiperidin-1-yl)pyridin-3-yl]-6-ethoxypyrazolo[1,5-a]pyridine-3-carbonitrile

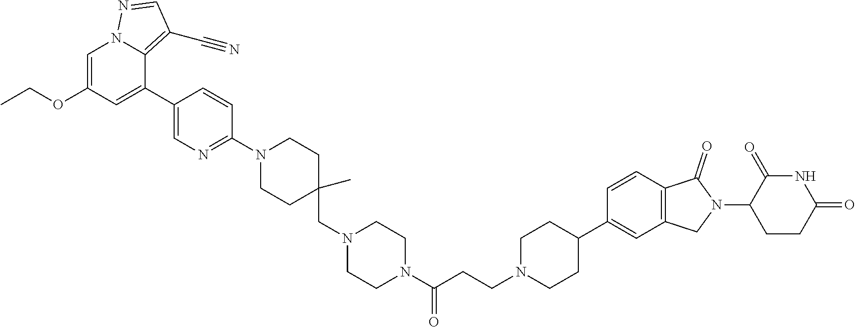 [SMILES: CCOc1cc(-c2ccc(N3CCC(C)(CN4CCN(C(=O)CCN5CCC(c6ccc7c(c6)CN(C6CCC(=O)NC6=O)C7=O)CC5)CC4)CC3)nc2)c2c(C#N)cnn2c1]

### Example 130

### 4-[6-(4-{[4-(7-{4-[2-(2,6-dioxopiperidin-3-yl)-1-oxo-2,3-dihydro-1H-isoindol-5-yl]piperazin-1-yl}heptanoyl)piperazin-1-yl]methyl}-4-ethylpiperidin-1-yl)pyridin-3-yl]-6-ethoxypyrazolo[1,5-a]pyridine-3-carbonitrile

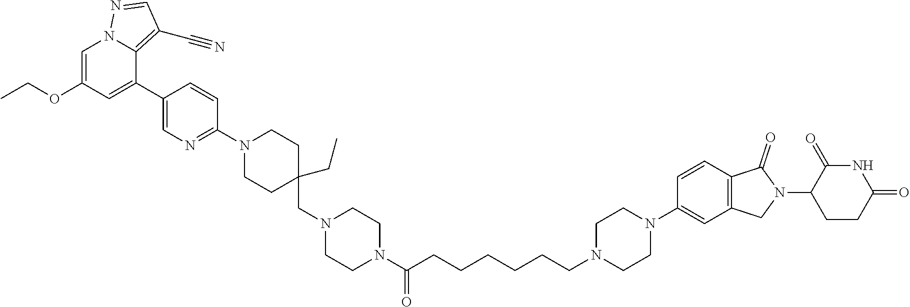 [SMILES: CCOc1cc(-c2ccc(N3CCC(CC)(CN4CCN(C(=O)CCCCCCN5CCN(c6ccc7c(c6)CN(C6CCC(=O)NC6=O)C7=O)CC5)CC4)CC3)nc2)c2c(C#N)cnn2c1]

### Example 131

### 4-[6-(4-{[4-(3-{4-[2-(2,6-dioxopiperidin-3-yl)-1-oxo-2,3-dihydro-1H-isoindol-5-yl]piperidin-1-yl}propanoyl)piperazin-1-yl]methyl}-4-ethylpiperidin-1-yl)pyridin-3-yl]-6-ethoxypyrazolo[1,5-a]pyridine-3-carbonitrile

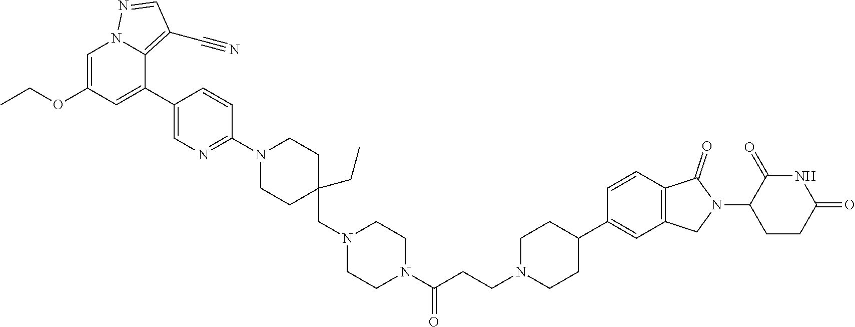 [SMILES: CCOc1cc(-c2ccc(N3CCC(CC)(CN4CCN(C(=O)CCN5CCC(c6ccc7c(c6)CN(C6CCC(=O)NC6=O)C7=O)CC5)CC4)CC3)nc2)c2c(C#N)cnn2c1]

### Example 132

### 4-[6-(4-{[4-(3-{4-[2-(2,6-dioxopiperidin-3-yl)-3-oxo-2,3-dihydro-1H-isoindol-5-yl]piperidin-1-yl}propanoyl)piperazin-1-yl]methyl}-4-ethylpiperidin-1-yl)pyridin-3-yl]-6-ethoxypyrazolo[1,5-a]pyridine-3-carbonitrile

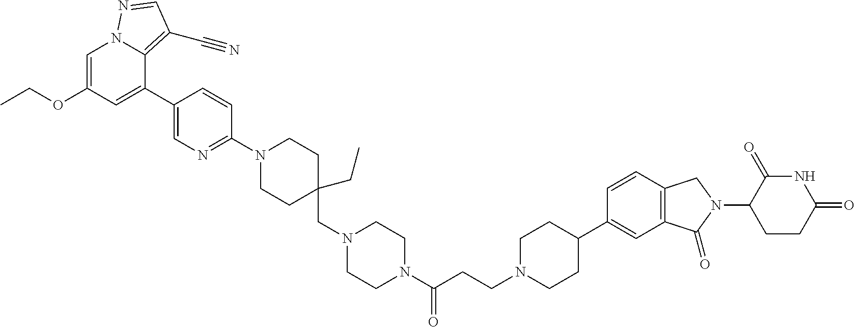 [SMILES: CCOc1cc(-c2ccc(N3CCC(CC)(CN4CCN(C(=O)CCN5CCC(c6ccc7c(c6)C(=O)N(C6CCC(=O)NC6=O)C7)CC5)CC4)CC3)nc2)c2c(C#N)cnn2c1]

### Example 133

### 4-[6-(4-{[4-(2-{[2-(2,6-dioxopiperidin-3-yl)-1-oxo-2,3-dihydro-1H-isoindol-5-yl]oxy}acetyl)piperazin-1-yl]methyl}-4-ethylpiperidin-1-yl)pyridin-3-yl]-6-ethoxypyrazolo[1,5-a]pyridine-3-carbonitrile

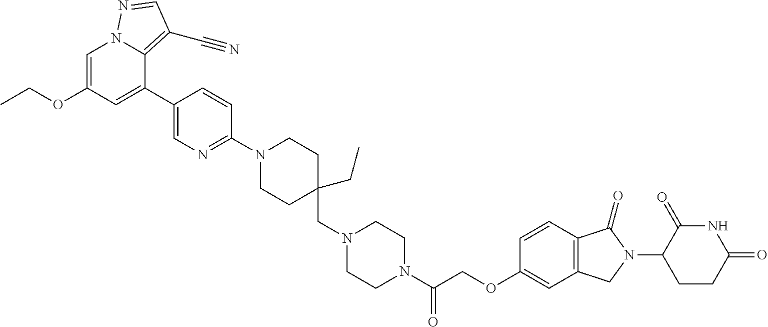 [SMILES: CCOc1cc(-c2ccc(N3CCC(CC)(CN4CCN(C(=O)COc5ccc6c(c5)CN(C5CCC(=O)NC5=O)C6=O)CC4)CC3)nc2)c2c(C#N)cnn2c1]

### Example 134

### N-(1-(5-(3-cyano-6-ethoxypyrazolo[1,5-a]pyridin-4-yl)pyridin-2-yl)-4-(9-(2-(2,6-dioxopiperidin-3-yl)-1-oxoisoindolin-5-yl)-3,9-diazaspiro[5.5]undecane-3-carbonyl)piperidin-4-yl)-2,5-difluorobenzamide

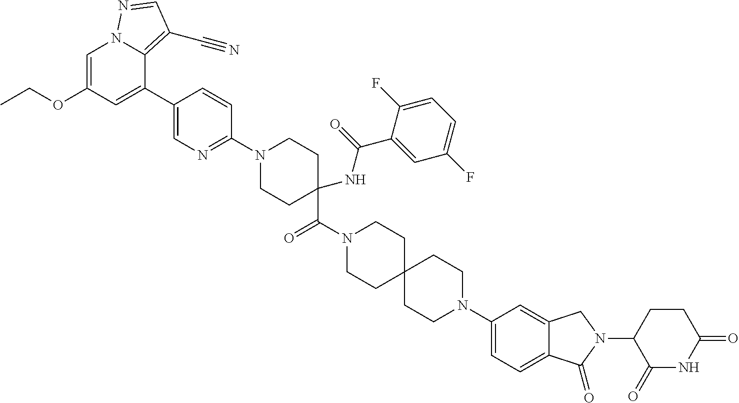 [SMILES: CCOc1cc(-c2ccc(N3CCC(NC(=O)c4cc(F)ccc4F)(C(=O)N4CCC5(CC4)CCN(c4ccc6c(c4)CN(C4CCC(=O)NC4=O)C6=O)CC5)CC3)nc2)c2c(C#N)cnn2c1]

### Example 135

### N-[1-(5-{3-cyano-6-ethoxypyrazolo[1,5-a]pyridin-4-yl}pyridin-2-yl)-4-{[4-(7-{4-[2-(1-methyl-2,6-dioxopiperidin-3-yl)-1-oxo-2,3-dihydro-1H-isoindol-5-yl]piperazin-1-yl}heptanoyl)piperazin-1-yl]methyl}piperidin-4-yl]-2,5-difluorobenzamide

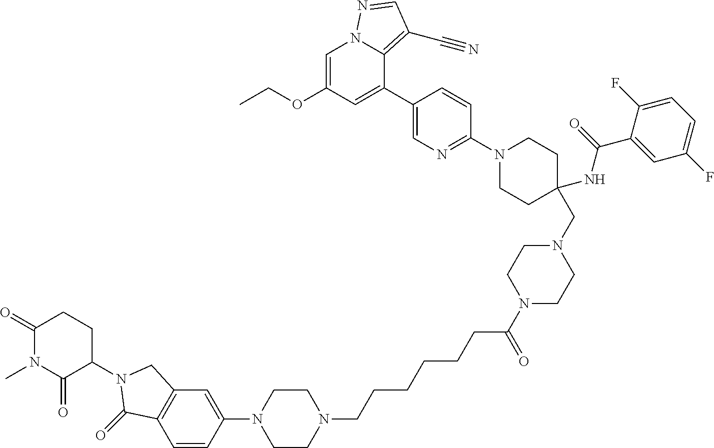 [SMILES: CCOc1cc(-c2ccc(N3CCC(CN4CCN(C(=O)CCCCCCN5CCN(c6ccc7c(c6)CN(C6CCC(=O)N(C)C6=O)C7=O)CC5)CC4)(NC(=O)c4cc(F)ccc4F)CC3)nc2)c2c(C#N)cnn2c1]

| | Analytical | |
|---|---|---|
| | LCMS | |
| | ESI- | |
| | MS(+) | |
| | m/z/RT | |
| Example | (min) | |
| # | (Method); | ¹H NMR |
| 100 | 1027.4 | ¹H NMR (500 MHz, DMSO-d6) δ 10.93 (s, 1H), 8.62 (d, J = 2.0 |
| | [M + H]⁺, | Hz, 1H), 8.55 (s, 1H), 8.31 (d, J = 2.6 Hz, 1H), 8.13 (s, 1H), 7.75 |
| | RT = 1.38 | (dd, J = 8.9, 2.6 Hz, 1H), 7.54 (d, J = 8.5 Hz, 1H), 7.38-7.30 (m, |
| | min (B)) | 3H), 7.25 (d, J = 2.1 Hz, 1H), 7.07-7.03 (m, 2H), 6.97 (d, J = 8.7 |
| | | Hz, 1H), 5.03 (dd, J = 13.2, 5.0 Hz, 1H), 4.36-4.29 (m, 1H), 4.24- |
| | | 4.11 (m, 5H), 3.75-3.66 (m, 3H), 3.65-3.60 (m, 2H), 3.37-3.24 |
| | | (m, 2H), 3.22-3.10 (m, 2H), 3.00-2.79 (m, 5H), 2.71-2.64 (m, |
| | | 3H), 2.62-2.54 (m, 13H), 2.41-2.26 (m, 3H), 2.00-1.91 (m, |
| | | 1H), 1.70-1.58 (m, 2H), 1.37 (t, J = 6.9 Hz, 3H) |
| 101 | 1026.6 | ¹H NMR (500 MHz, DMSO-d6) δ 10.97 (s, 1H), 8.62 (d, J = 1.8 |
| | [M + H]⁺, | Hz, 1H), 8.55 (s, 1H), 8.31 (d, J = 2.6 Hz, 1H), 8.13 (s, 1H), 7.76 |
| | RT = 1.42 | (dd, J = 8.7, 2.6 Hz, 1H), 7.60-7.48 (m, 3H), 7.38-7.31 (m, 3H), |
| | min (B) | 7.25 (d, J = 2.1 Hz, 1H), 7.01-6.93 (m, 1H), 5.08 (dd, J = 13.3, 5.1 |
| | | Hz, 1H), 4.60-4.52 (m, 1H), 4.44-4.37 (m, 1H), 4.31-4.25 (m, |
| | | 1H), 4.21-4.11 (m, 4H), 4.04-3.96 (m, 1H), 3.77-3.66 (m, 2H), |
| | | 3.34-3.26 (m, 1H), 3.21-3.08 (m, 3H), 3.00-2.79 (m, 5H), 2.70- |
| | | 2.60 (m, 4H), 2.60-2.54 (m, 10H), 2.41-2.26 (m, 3H), 2.04-1.94 |
| | | (m, 1H), 1.89-1.78 (m, 2H), 1.70-1.58 (m, 3H), 1.52-1.41 |
| | | (m, 1H), 1.37 (t, J = 6.9 Hz, 3H) |
| 102 | 958.3 | ¹H NMR (500 MHz, DMSO-d6) δ 10.52 (s, 1H), 10.28 (s, 1H), |
| | [M + H]⁺, | 8.62 (d, J = 2.1 Hz, 1H), 8.55 (s, 1H), 8.31 (d, J = 2.4 Hz, 1H), 8.17- |
| | RT = 1.36 | 8.01 (m, 2H), 7.76 (dd, J = 8.9, 2.6 Hz, 1H), 7.57 (d, J = 8.9 Hz, |
| | min (B) | 1H), 7.38-7.29 (m, 3H), 7.25 (d, J = 2.1 Hz, 1H), 7.08-7.06 (m, |
| | | 1H), 6.96 (d, J = 9.2 Hz, 1H), 4.18-4.10 (m, 4H), 3.92-3.87 (m, |
| | | 4H), 3.80-3.72 (m, 2H), 3.31-3.23 (m, 1H), 3.19-3.07 (m, 2H), |
| | | 2.76-2.69 (m, 4H), 2.68-2.54 (m, 14H), 2.34-2.17 (m, 2H), |
| | | 1.69-1.49 (m, 2H), 1.37 (t, J = 6.9 Hz, 3H) |
| 103 | 1027.6 | ¹H NMR (500 MHz, DMSO-d6) δ 10.96 (s, 1H), 8.69-8.61 (m, |
| | [M + H]⁺, | 1H), 8.60-8.53 (m, 1H), 8.32 (s, 1H), 8.16 (s, 1H), 7.89-7.76 |
| | RT = 1.38 | (m, 1H), 7.45 (d, J = 8.6 Hz, 1H), 7.41-7.31 (m, 3H), 7.30-7.25 |
| | min (B) | (m, 2H), 7.22-7.18 (m, 1H), 7.09-7.02 (m, 1H), 5.11-5.04 (m, |
| | | 1H), 4.34 (br d, J = 17.0 Hz, 1H), 4.28-4.01 (m, 5H), 3.78-3.57 |
| | | (m, 2H), 3.33-3.28 (m, 1H), 3.21-3.21 (m, 1H), 3.27-3.11 (m, |
| | | 4H), 2.84-2.84 (m, 1H), 3.04-2.75 (m, 4H), 2.72-2.64 (m, 2H), |
| | | 2.54 (s, 15H), 2.45-2.27 (m, 3H), 2.04-1.94 (m, 1H), 1.71- |
| | | 1.61 (m, 2H), 1.38 (t, J = 6.9 Hz, 3H) |
| 104 | 1027.2 | ¹H NMR (400 MHz, DMSO-d6) d 10.93 (br s, 1H), 8.64 (d, J = 2.0 |
| | [M + H]⁺, | Hz, 1H), 8.57 (s, 1H), 8.32 (d, J = 3.0 Hz, 1H), 8.12 (s, 1H), 7.75 |
| | RT = 1.213 | (dd, J = 8.9, 2.5 Hz, 1H), 7.55-7.47 (m, 1H), 7.38-7.30 (m, 3H), |
| | min (B) | 7.26 (d, J = 2.0 Hz, 1H), 7.08-7.02 (m, 2H), 6.96 (d, J = 8.8 Hz, |
| | | 1H), 5.04 (dd, J = 13.3, 5.2 Hz, 1H), 4.36-4.11 (m, 6H), 3.61 (t, |
| | | J = 6.6 Hz, 2H), 3.56-3.49 (m, 2H), 3.42 (br d, J = 1.7 Hz, 4H), |
| | | 3.29-3.24 (m, 4H), 3.22-3.11 (m, 2H), 2.97-2.82 (m, 1H), 2.73 |
| | | (s, 2H), 2.61-2.52 (m, 9H), 2.48-2.43 (m, 3H), 2.39-2.27 (m, |
| | | 4H), 2.00-1.91 (m, 1H), 1.67-1.55 (m, 2H), 1.38 (t, J = 7.0 Hz, |
| | | 3H) |
| 105 | 958.3 | ¹H NMR (400 MHz, DMSO-d6) δ 10.44 (br s, 1H), 8.64 (d, J = 2.0 |
| | [M + H]⁺, | Hz, 1H), 8.57 (s, 1H), 8.32 (d, J = 2.9 Hz, 1H), 8.10 (s, 1H), 7.75 |
| | RT = | (dd, J = 8.8, 2.6 Hz, 1H), 7.39-7.24 (m, 5H), 6.97 (d, J = 8.8 Hz, |
| | 1.319 min | 1H), 6.54 (dd, J = 8.9, 1.9 Hz, 1H), 6.37 (d, J = 1.8 Hz, 1H), 5.90 (t, |
| | (B) | J = 5.6 Hz, 1H), 4.23-4.12 (m, 4H), 3.89-3.83 (m, 2H), 3.81 (s, |
| | | 3H), 3.67 (t, J = 6.5 Hz, 2H), 3.61 (t, J = 5.7 Hz, 2H), 3.47-3.37 |
| | | (m, 4H), 3.24 (q, J = 5.4 Hz, 2H), 3.20-3.10 (m, 2H), 2.74-2.65 |
| | | (m, 4H), 2.58 (t, J = 6.5 Hz, 2H), 2.49-2.42 (m, 4H), 2.37-2.28 |
| | | (m, 2H), 1.65-1.54 (m, 2H), 1.38 (t, J = 7.0 Hz, 3H) |
| 106 | 885.4 | ¹H NMR (500 MHz, DMSO-d6) δ 10.97 (s, 1H), 8.63 (d, J = 2.0 |
| | [M + H]⁺, | Hz, 1H), 8.56 (s, 1H), 8.31 (d, J = 2.4 Hz, 1H), 8.11 (s, 1H), 7.74 |
| | RT = 1.35 | (dd, J = 8.7, 2.6 Hz, 1H), 7.58 (s, 1H), 7.55-7.51 (m, 1H), 7.49- |
| | min (B) | 7.45 (m, 1H), 7.37-7.28 (m, 3H), 7.26 (d, J = 2.1 Hz, 1H), 6.96 (d, |
| | | J = 9.1 Hz, 1H), 5.09 (dd, J = 13.4, 5.1 Hz, 1H), 4.47-4.26 (m, |
| | | 2H), 4.21-4.12 (m, 4H), 3.83 (s, 2H), 3.19-3.10 (m, 1H), 2.96-2.84 |
| | | (m, 1H), 2.74-2.54 (m, 7H), 2.45-2.27 (m, 4H), 2.05-1.96 (m, 1H), |
| | | 1.64-1.55 (m, 2H), 1.38 (t, J = 6.9 Hz, 3H). Integration low |
| | | due to overlap with suppressed water. |
| 107 | 901.2 | ¹H NMR (500 MHz, DMSO-d6) δ 10.96 (s, 1H), 8.66 (d, J = 1.9 |
| | [M + H]⁺, | Hz, 1H), 8.58 (s, 1H), 8.54-8.42 (m, 1H), 8.36 (d, J = 2.3 Hz, 1H), |
| | RT = 1.42 | 7.84 (dd, J = 8.6, 2.3 Hz, 1H), 7.64 (d, J = 8.4 Hz, 1H), 7.56-7.47 |
| | min (B) | (m, 1H), 7.47-7.36 (m, 2H), 7.28 (d, J = 1.9 Hz, 1H), 7.17-7.14 |
| | | (m, 1H), 7.10-7.05 (m, 2H), 5.07 (dd, J = 13.5, 5.0 Hz, 1H), 5.00 |
| | | (br s, 2H), 4.42-4.35 (m, 1H), 4.31-4.23 (m, 3H), 4.16 (q, J = 6.9 |
| | | Hz, 2H), 3.28-3.17 (m, 2H), 2.97-2.84 (m, 1H), 2.64-2.54 (m, |
| | | 10H), 2.49-2.32 (m, 4H), 2.04-1.93 (m, 1H), 1.78-1.69 (m, |
| | | 2H), 1.39 (t, J = 7.0 Hz, 3H) |
| 108 | 1002.2 | ¹H NMR (500 MHz, DMSO-d6) δ 10.16 (s, 1H), 8.60 (d, J = 1.9 |
| | [M + H]⁺, | Hz, 1H), 8.54 (s, 1H), 8.31 (d, J = 2.4 Hz, 1H), 8.08 (s, 1H), 8.04 |
| | RT = 1.40 | (s, 1H), 7.73 (dd, J = 8.8, 2.5 Hz, 1H), 7.55 (d, J = 8.9 Hz, 1H), |
| | min (B) | 7.35-7.27 (m, 3H), 7.24 (d, J = 1.9 Hz, 1H), 7.06 (d, J = 9.0 Hz, 1H), |
| | | 6.98-6.90 (m, 1H), 4.14 (q, J = 6.9 Hz, 4H), 3.93-3.86 (m, 5H), |
| | | 3.71 (br t, J = 6.0 Hz, 2H), 3.19-3.07 (m, 2H), 2.73 (t, J = 6.6 Hz, |
| | | 2H), 2.66-2.61 (m, 2H), 2.61-2.56 (m, 2H), 2.48-2.43 (m, 3H), |
| | | 2.39-2.24 (m, 7H), 1.85 (s, 1H), 1.59-1.50 (m, 2H), 1.37 (t, |
| | | J = 6.9 Hz, 3H). Integration low due to overlap with suppressed |
| | | water and obscurement by DMSO signal. |
| 109 | 1071.3 | ¹H NMR (500 MHz, DMSO-d6) δ 10.93 (s, 1H), 8.62 (d, J = 1.8 |
| | [M + H]⁺, | Hz, 1H), 8.56 (s, 1H), 8.31 (d, J = 2.4 Hz, 1H), 8.12-8.02 (m, 1H), |
| | RT = 1.40 | 7.74 (dd, J = 8.7, 2.3 Hz, 1H), 7.53 (d, J = 8.3 Hz, 1H), 7.37-7.28 |
| | min (B) | (m, 3H), 7.25 (d, J = 2.0 Hz, 1H), 7.09-7.02 (m, 2H), 6.99-6.91 |
| | | (m, 1H), 5.03 (dd, J = 13.2, 4.8 Hz, 1H), 4.36-4.18 (m, 2H), 4.18-4.10 |
| | | (m, 4H), 3.68-3.57 (m, 5H), 3.53-3.44 (m, 2H), 3.37-3.23 (m, 1H), |
| | | 3.20-3.10 (m, 2H), 2.95-2.83 (m, 1H), 2.70-2.56 (m, 5H), 2.46-2.25 |
| | | (m, 8H), 2.00-1.91 (m, 1H), 1.65-1.50 (m, 2H), 1.38 (t, J = 6.9 Hz, |
| | | 3H). Integration low due to overlap with suppressed water and |
| | | obscurement by DMSO signal. |
| 110 | 968.3 | ¹H NMR (500 MHz, DMSO-d6) δ 11.00 (s, 1H), 8.66 (d, J = 1.9 |
| | [M + H]⁺, | Hz, 1H), 8.57 (s, 1H), 8.33 (d, J = 2.5 Hz, 1H), 8.23-8.13 (m, 1H), |
| | RT = 1.41 | 7.86-7.80 (m, 1H), 7.72-7.67 (m, 2H), 7.64 (d, J = 7.6 Hz, 1H), |
| | min (B) | 7.43-7.31 (m, 3H), 7.28 (d, J = 2.0 Hz, 1H), 7.10-7.05 (m, 1H), |
| | | 5.12 (dd, J = 13.6, 4.9 Hz, 1H), 4.55-4.35 (m, 2H), 4.25-4.11 (m, |
| | | 4H), 3.28-3.15 (m, 2H), 3.04-2.97 (m, 2H), 2.96-2.85 (m, 4H), |
| | | 2.75-2.57 (m, 2H), 2.47-2.28 (m, 4H), 2.16-1.97 (m, 2H), 1.93- |
| | | 1.61 (m, 4H), 1.38 (t, J = 7.0 Hz, 3H), 1.32-1.16 (m, 2H). |
| | | Integration low due to overlap with suppressed water and |
| | | obscurement by DMSO signal. |
| 111 | 982.3 | ¹H NMR (500 MHz, DMSO-d6) δ 10.99 (s, 1H), 8.64 (d, J = 2.0 |
| | [M + H]⁺, | Hz, 1H), 8.56 (s, 1H), 8.32 (d, J = 2.4 Hz, 1H), 8.17 (s, 1H), 7.83 |
| | RT = 1.30 | (dd, J = 8.8, 2.5 Hz, 1H), 7.62 (d, J = 7.8 Hz, 1H), 7.50-7.40 (m, |
| | min (B) | 2H), 7.39-7.32 (m, 3H), 7.28 (d, J = 2.0 Hz, 1H), 7.09-7.05 (m, |
| | | 1H), 5.15-5.08 (m, 1H), 4.46-4.26 (m, 3H), 4.23-4.10 (m, 4H), |
| | | 4.04-3.94 (m, 1H), 3.91-3.79 (m, 2H), 3.26-3.16 (m, 2H), 3.10- |
| | | 3.01 (m, 1H), 3.00-2.85 (m, 6H), 2.66-2.57 (m, 3H), 2.43- |
| | | 2.28 (m, 3H), 2.10-1.96 (m, 2H), 1.79-1.60 (m, 4H), 1.38 (t, |
| | | J = 7.0 Hz, 3H), 1.11-0.97 (m, 2H). Integration low due to |
| | | overlap with suppressed water and obscurement by DMSO signal. |
| 112 | 982.3 | ¹H NMR (500 MHz, DMSO-d6) δ 10.98 (s, 1H), 8.66 (d, J = 1.9 |
| | [M + H]⁺, | Hz, 1H), 8.57 (s, 1H), 8.33 (d, J = 2.2 Hz, 1H), 8.16 (s, 1H), 7.86- |
| | RT = 1.39 | 7.81 (m, 1H), 7.67 (d, J = 7.8 Hz, 1H), 7.46 (s, 1H), 7.39-7.34 (m, |
| | min (B) | 4H), 7.29 (d, J = 1.8 Hz, 1H), 7.08 (br d, J = 9.3 Hz, 1H), 5.10 (dd, |
| | | J = 13.2, 4.9 Hz, 1H), 4.46-4.41 (m, 1H), 4.40-4.34 (m, 1H), |
| | | 4.32-4.28 (m, 1H), 4.20-4.13 (m, 4H), 4.02-3.94 (m, 1H), 3.87-3.82 |
| | | (m, 2H), 3.25-3.19 (m, 2H), 3.07-2.86 (m, 8H), 2.64-2.56 (m, 3H), |
| | | 2.45-2.29 (m, 4H), 2.05-1.97 (m, 2H), 1.74-1.63 (m, 4H), 1.38 |
| | | (t, J = 6.9 Hz, 3H), 1.05-0.94 (m, 2H). Integration low due |
| | | to overlap with suppressed water and obscurement by |
| | | DMSO signal. |
| 113 | 982.3 | ¹H NMR (500 MHz, DMSO-d6) δ 10.98 (s, 1H), 8.66 (d, J = 1.8 |
| | [M + H]⁺, | Hz, 1H), 8.57 (s, 1H), 8.33 (d, J = 2.3 Hz, 1H), 8.17 (s, 1H), 7.85 |
| | RT = 1.29 | (dd, J = 9.0, 1.7 Hz, 1H), 7.61 (s, 1H), 7.56-7.52 (m, 1H), 7.51- |
| | min (B) | 7.47 (m, 1H), 7.41-7.33 (m, 3H), 7.29 (d, J = 1.2 Hz, 1H), 7.14- |
| | | 7.07 (m, 1H), 5.09 (br dd, J = 12.9, 4.8 Hz, 1H), 4.47-4.40 (m, |
| | | 1H), 4.40-4.34 (m, 1H), 4.33-4.27 (m, 1H), 4.22-4.13 (m, 4H), |
| | | 4.06-3.97 (m, 1H), 3.84 (br s, 2H), 3.28-3.18 (m, 2H), 3.06- |
| | | 2.87 (m, 7H), 2.65-2.56 (m, 3H), 2.45-2.30 (m, 3H), 2.05-1.98 |
| | | (m, 2H), 1.75-1.63 (m, 4H), 1.38 (t, J = 6.9 Hz, 3H), 1.05-0.95 |
| | | (m, 2H). Integration low due to overlap with suppressed water |
| | | and obscurement by DMSO signal. |
| 114 | 998.7 | ¹H NMR (500 MHz, DMSO-d6) δ 10.96 (s, 1H), 8.64 (d, J = 2.1 |
| | [M + H]⁺, | Hz, 1H), 8.56 (s, 1H), 8.32 (d, J = 2.6 Hz, 1H), 8.18-8.12 (m, 1H), |
| | RT = 1.34 | 7.80 (dd, J = 9.0, 2.4 Hz, 1H), 7.63 (d, J = 8.4 Hz, 1H), 7.39-7.33 |
| | min (B) | (m, 3H), 7.27 (d, J = 2.1 Hz, 1H), 7.13 (s, 1H), 7.04-7.00 (m, 2H), |
| | | 5.06 (dd, J = 13.2, 5.0 Hz, 1H), 4.99-4.85 (m, 2H), 4.42-4.35 (m, |
| | | 1H), 4.34-4.23 (m, 2H), 4.22-4.12 (m, 4H), 3.87-3.79 (m, 1H), |
| | | 3.24-3.14 (m, 2H), 3.10-3.02 (m, 1H), 3.01-2.83 (m, 6H), 2.70- |
| | | 2.56 (m, 3H), 2.43-2.28 (m, 3H), 2.11-1.93 (m, 2H), 1.80- |
| | | 1.72 (m, 2H), 1.70-1.60 (m, 2H), 1.38 (s, 3H), 1.28-0.98 (m, |
| | | 2H). Integration low due to overlap with suppressed water and |
| | | obscurement by DMSO signal. |
| 115 | 983.1 | ¹H NMR (500 MHz, DMSO-d₆) δ 10.96 (s, 1H), 8.69-8.61 (m, |
| | [M + H]⁺, | 1H), 8.56 (s, 1H), 8.32 (d, J = 2.5 Hz, 1H), 8.18-8.07 (m, 1H), |
| | RT = 1.36 | 7.76 (dd, J = 8.7, 2.5 Hz, 1H), 7.46 (d, J = 8.6 Hz, 1H), 7.39-7.28 |
| | min (B) | (m, 4H), 7.26 (d, J = 2.1 Hz, 1H), 7.23-7.18 (m, 1H), 6.97 (d, |
| | | J = 8.9 Hz, 1H), 5.11-5.05 (m, 1H), 4.35 (d, J = 16.7 Hz, 1H), |
| | | 4.26-4.11 (m, 5H), 3.89 (s, 1H), 3.66-3.60 (m, 2H), 3.38-3.12 (m, |
| | | 4H), 3.00-2.77 (m, 5H), 2.66-2.53 (m, 3H), 2.44-2.27 (m, 3H), |
| | | 2.04-1.95 (m, 1H), 1.69-1.58 (m, 2H), 1.38 (t, J = 7.0 Hz, 3H), |
| | | 1.32-1.20 (m, 2H), 0.90-0.83 (m, 1H). Integration low due to |
| | | overlap with suppressed water and obscurement by DMSO signal. |
| 116 | 982.5 | ¹H NMR (500 MHz, DMSO-d₆) δ 8.61 (d, J = 1.8 Hz, 1H), 8.54 (s, |
| | [M + H]⁺, | 1H), 8.31 (d, J = 2.2 Hz, 1H), 8.15-8.03 (m, 1H), 7.74 (dd, J = 8.7, |
| | RT = 1.32 | 1.9 Hz, 1H), 7.60-7.48 (m, 3H), 7.36-7.27 (m, 3H), 7.25 (d, |
| | min (B) | J = 2.0 Hz, 1H), 6.95 (br d, J = 9.3 Hz, 1H), 5.08 (dd, J = 13.3, 4.7 |
| | | Hz, 1H), 4.57-4.50 (m, 1H), 4.44-4.24 (m, 2H), 4.21-4.10 (m, |
| | | 4H), 4.05-3.95 (m, 1H), 3.20-3.08 (m, 2H), 2.98-2.84 (m, 2H), |
| | | 2.76-2.66 (m, 2H), 2.65-2.57 (m, 2H), 2.43-2.27 (m, 5H), 2.04- |
| | | 1.95 (m, 1H), 1.85-1.73 (m, 3H), 1.71-1.54 (m, 3H), 1.50- |
| | | 1.41 (m, 1H), 1.37 (t, J = 7.0 Hz, 3H). Integration low due to |
| | | overlap with suppressed water and obscurement by DMSO signal. |
| 117 | 996.5 | ¹H NMR (500 MHz, DMSO-d₆) δ 11.11 (s, 1H), 8.64 (d, J = 2.0 |
| | [M + H]⁺, | Hz, 1H), 8.56 (s, 1H), 8.32 (d, J = 2.4 Hz, 1H), 8.14 (s, 1H), 7.87 |
| | RT = 1.45 | (d, J = 7.7 Hz, 1H), 7.81 (s, 1H), 7.79-7.75 (m, 2H), 7.39-7.32 |
| | min (B) | (m, 3H), 7.26 (d, J = 2.0 Hz, 1H), 7.02-6.97 (m, 1H), 5.13 (dd, |
| | | J = 13.4, 5.4 Hz, 1H), 4.59-4.50 (m, 1H), 4.24-4.12 (m, 4H), |
| | | 4.04-3.95 (m, 1H), 3.24-2.81 (m, 11H), 2.75-2.58 (m, 4H), |
| | | 2.37-2.28 (m, 2H), 2.10-2.00 (m, 1H), 1.92-1.82 (m, 2H), 1.73- |
| | | 1.61 (m, 3H), 1.60-1.47 (m, 1H), 1.38 (t, J = 7.0 Hz, 3H), 1.31- |
| | | 1.21 (m, 1H), 1.16 (t, J = 7.4 Hz, 1H). Integration low due to |
| | | overlap with suppressed water and obscurement by DMSO signal. |
| 118 | 998.3 | ¹H NMR (500 MHz, DMSO-d₆) δ 10.96 (s, 1H), 8.69-8.64 (m, |
| | [M + H]⁺, | 1H), 8.61-8.47 (m, 2H), 8.36 (d, J = 2.5 Hz, 1H), 7.85 (dd, J = 8.9, |
| | RT = 1.20 | 2.6 Hz, 1H), 7.66 (dd, J = 11.2, 8.5 Hz, 1H), 7.53-7.47 (m, 1H), |
| | min (B) | 7.45-7.37 (m, 2H), 7.30-7.22 (m, 2H), 7.19-7.08 (m, 2H), 5.11- |
| | | 5.04 (m, 1H), 4.43-4.36 (m, 1H), 4.34-4.23 (m, 3H), 4.16 (q, |
| | | J = 7.1 Hz, 2H), 3.47-3.32 (m, 2H), 3.29-3.20 (m, 2H), 3.18- |
| | | 3.10 (m, 1H), 2.95-2.86 (m, 2H), 2.65-2.57 (m, 2H), 2.48-2.29 |
| | | (m, 4H), 2.16-1.95 (m, 3H), 1.90-1.70 (m, 3H), 1.38 (t, J = 7.0 |
| | | Hz, 3H). Integration low due to overlap with suppressed water |
| | | and obscurement by DMSO signal. |
| 119 | 954.1 | ¹H NMR (500 MHz, DMSO-d₆) δ 8.60 (d, J = 1.9 Hz, 1H), 8.54 (s, |
| | [M + H]⁺, | 1H), 8.31 (d, J = 2.4 Hz, 1H), 8.12 (s, 1H), 7.74 (dd, J = 8.9, 2.4 Hz, |
| | RT = 1.29 | 1H), 7.66 (d, J = 7.9 Hz, 1H), 7.56 (s, 1H), 7.51-7.42 (m, 1H), |
| | min (B) | 7.38-7.28 (m, 3H), 7.24 (d, J = 2.0 Hz, 1H), 6.96 (d, J = 9.0 Hz, |
| | | 1H), 5.12-5.03 (m, 1H), 4.47-4.26 (m, 2H), 4.21-4.11 (m, 4H), |
| | | 3.75-3.58 (m, 2H), 3.57-3.35 (m, 1H), 3.19-3.07 (m, 4H), 2.95- |
| | | 2.83 (m, 1H), 2.72 (s, 2H), 2.65-2.56 (m, 3H), 2.48-2.42 (m, |
| | | 2H), 2.41-2.35 (m, 1H), 2.35-2.29 (m, 4H), 2.04-1.95 (m, 1H), |
| | | 1.89-1.83 (m, 4H), 1.66-1.55 (m, 2H), 1.37 (t, J = 6.9 Hz, 3H). |
| | | Integration low due to overlap with suppressed water and |
| | | obscurement by DMSO signal. |
| 120 | 970.3 | ¹H NMR (500 MHz, DMSO-d₆) δ 10.96 (s, 1H), 8.63 (d, J = 2.0 |
| | [M + H]⁺, | Hz, 1H), 8.59-8.47 (m, 2H), 8.39-8.30 (m, 1H), 7.83 (dd, J = 8.8, |
| | RT = 1.19 | 2.4 Hz, 1H), 7.73-7.66 (m, 1H), 7.51-7.44 (m, 1H), 7.42-7.32 |
| | min (B) | (m, 2H), 7.27 (d, J = 1.9 Hz, 1H), 7.12-7.05 (m, 2H), 7.03-7.00 |
| | | (m, 1H), 5.06 (dd, J = 13.5, 5.1 Hz, 1H), 4.77-4.47 (m, 2H), 4.45- |
| | | 4.36 (m, 1H), 4.32-4.19 (m, 4H), 4.15 (q, J = 7.0 Hz, 2H), 3.26- |
| | | 3.17 (m, 2H), 2.95-2.84 (m, 1H), 2.82-2.70 (m, 2H), 2.64-2.52 |
| | | (m, 12H), 2.46-2.30 (m, 3H), 2.02-1.95 (m, 1H), 1.79-1.66 |
| | | (m, 2H). Integration low due to overlap with suppressed water |
| | | and obscurement by DMSO signal. |
| 121 | 1035.4 | ¹H NMR (500 MHz, DMSO-d₆) δ 10.97 (s, 1H), 8.65 (d, J = 1.9 |
| | [M + H]⁺, | Hz, 1H), 8.59-8.51 (m, 2H), 8.35 (d, J = 2.3 Hz, 1H), 7.82 (dd, |
| | RT = 1.66 | J = 8.8, 2.2 Hz, 1H), 7.57 (s, 1H), 7.54-7.49 (m, 3H), 7.44-7.36 |
| | min (B) | (m, 2H), 7.27 (d, J = 1.8 Hz, 1H), 7.07 (br d, J = 9.0 Hz, 1H), |
| | | 5.13-5.06 (m, 1H), 4.61-4.55 (m, 1H), 4.45-4.39 (m, 1H), 4.32-4.24 |
| | | (m, 3H), 4.16 (q, J = 7.1 Hz, 2H), 4.06-3.99 (m, 1H), 3.27-3.06 |
| | | (m, 4H), 2.97-2.85 (m, 2H), 2.70-2.54 (m, 4H), 2.47-2.34 (m, |
| | | 4H), 2.27 (br dd, J = 7.1, 1.1 Hz, 2H), 2.07-1.93 (m, 3H), 1.89- |
| | | 1.79 (m, 2H), 1.78-1.64 (m, 4H), 1.60-1.34 (m, 11H), 1.30- |
| | | 0.99 (m, 5H). |
| 122 | 1035.5 | ¹H NMR (500 MHz, DMSO-d₆) δ 10.97 (s, 1H), 8.65 (d, J = 1.9 |
| | [M + H]⁺, | Hz, 1H), 8.61-8.52 (m, 2H), 8.35 (d, J = 2.6 Hz, 1H), 7.81 (dd, |
| | RT = 1.64 | J = 8.8, 2.6 Hz, 1H), 7.66 (d, J = 7.9 Hz, 1H), 7.54-7.46 (m, 2H), |
| | min (B) | 7.46-7.35 (m, 3H), 7.27 (d, J = 2.1 Hz, 1H), 7.04 (d, J = 5.2 Hz, |
| | | 1H), 5.09 (dd, J = 13.2, 5.1 Hz, 1H), 4.62-4.54 (m, 1H), 4.45- |
| | | 4.38 (m, 1H), 4.33-4.23 (m, 3H), 4.16 (q, J = 6.9 Hz, 2H), 4.07- |
| | | 3.97 (m, 1H), 3.67-3.59 (m, 1H), 3.26-3.07 (m, 4H), 2.97-2.85 |
| | | (m, 2H), 2.65-2.57 (m, 3H), 2.47-2.32 (m, 4H), 2.27 (br d, |
| | | J = 6.7 Hz, 2H), 2.06-1.92 (m, 3H), 1.88-1.78 (m, 2H), 1.77- |
| | | 1.64 (m, 4H), 1.62-1.32 (m, 11H), 1.30-0.97 (m, 5H). |
| 123 | 1036.4 | ¹H NMR (500 MHz, DMSO-d₆) δ 10.96 (s, 1H), 8.65 (d, J = 2.0 |
| | [M + H]⁺, | Hz, 1H), 8.59-8.51 (m, 2H), 8.35 (d, J = 2.4 Hz, 1H), 7.83 (dd, |
| | RT = 1.47 | J = 8.9, 2.6 Hz, 1H), 7.53-7.49 (m, 1H), 7.46 (d, J = 8.3 Hz, 1H), |
| | min (B) | 7.44-7.36 (m, 2H), 7.30-7.26 (m, 2H), 7.20 (d, J = 2.0 Hz, 1H), |
| | | 7.08 (d, J = 8.9 Hz, 1H), 5.08 (dd, J = 13.3, 5.1 Hz, 1H), 4.39-4.31 |
| | | (m, 1H), 4.30-4.20 (m, 3H), 4.16 (q, J = 7.0 Hz, 2H), 3.66-3.60 |
| | | (m, 1H), 3.44-3.35 (m, 1H), 3.26-3.13 (m, 6H), 2.96-2.85 (m, |
| | | 1H), 2.64-2.54 (m, 6H), 2.48-2.32 (m, 4H), 2.28 (br d, J = 6.2 |
| | | Hz, 2H), 2.05-1.92 (m, 3H), 1.79-1.62 (m, 4H), 1.60-1.32 (m, |
| | | 9H), 1.30-0.98 (m, 5H). |
| 124 | 1036.4 | ¹H NMR (500 MHz, DMSO-d₆) δ 10.94 (s, 1H), 8.65 (d, J = 1.8 |
| | [M + H]⁺, | Hz, 1H), 8.58-8.52 (m, 2H), 8.35 (d, J = 2.4 Hz, 1H), 7.82 (dd, |
| | RT = 1.45 | J = 9.0, 2.5 Hz, 1H), 7.57-7.48 (m, 2H), 7.45-7.36 (m, 2H), 7.27 |
| | min (B) | (d, J = 2.0 Hz, 1H), 7.10-7.04 (m, 3H), 5.04 (dd, J = 13.2, 5.2 Hz, |
| | | 1H), 4.37-4.31 (m, 1H), 4.31-4.19 (m, 3H), 4.16 (q, J = 6.9 Hz, |
| | | 2H), 3.66-3.58 (m, 2H), 3.49-3.09 (m, 6H), 2.96-2.84 (m, 1H), |
| | | 2.63-2.54 (m, 6H), 2.48-2.31 (m, 4H), 2.28 (br d, J = 6.8 Hz, |
| | | 2H), 2.06-1.92 (m, 3H), 1.80-1.62 (m, 4H), 1.60-1.32 (m, 9H), |
| | | 1.31-0.97 (m, 5H). |
| 125 | 884.3 | ¹H NMR (500 MHz, DMSO-d₆) δ 10.95 (s, 1H), 8.63 (s, 1H), |
| | [M + H]⁺, | 8.56 (s, 1H), 8.32 (d, J = 2.1 Hz, 1H), 7.82-7.75 (m, 1H), 7.59 (br |
| | RT = 1.01 | d, J = 8.5 Hz, 1H), 7.26 (s, 1H), 7.20-7.11 (m, 2H), 7.00 (br d, |
| | min (B) | J = 8.8 Hz, 1H), 5.09-5.00 (m, 1H), 4.45-4.32 (m, 3H), 4.27- |
| | | 4.20 (m, 1H), 4.19-4.10 (m, 2H), 3.19-3.03 (m, 5H), 3.01-2.83 |
| | | (m, 4H), 2.65-2.54 (m, 3H), 2.42-2.31 (m, 3H), 2.20-2.07 (m, |
| | | 1H), 2.01-1.92 (m, 1H), 1.89-1.79 (m, 2H), 1.72-1.62 (m, 2H), |
| | | 1.57-1.46 (m, 2H), 1.41-1.17 (m, 11H). Integration low due to |
| | | overlap with suppressed water and obscurement by DMSO signal. |
| 126 | 827.3 | ¹H NMR (500 MHz, DMSO-d₆) δ 10.98 (s, 1H), 8.64 (d, J = 2.1 |
| | [M + H]⁺, | Hz, 1H), 8.56 (s, 1H), 8.32 (d, J = 2.4 Hz, 1H), 7.82-7.74 (m, 1H), |
| | RT = 1.09 | 7.63-7.56 (m, 2H), 7.55-7.50 (m, 1H), 7.27 (d, J = 2.1 Hz, 1H), |
| | min (B) | 7.00 (d, J = 8.8 Hz, 1H), 5.12-5.06 (m, 1H), 4.47-4.38 (m, 3H), |
| | | 4.34-4.27 (m, 1H), 4.15 (q, J = 7.3 Hz, 2H), 3.69-3.61 (m, 1H), |
| | | 3.20-3.05 (m, 4H), 3.05-2.83 (m, 6H), 2.65-2.54 (m, 2H), 2.47- |
| | | 2.34 (m, 1H), 2.21-2.11 (m, 1H), 2.10-1.91 (m, 5H), 1.89-1.82 |
| | | (m, 2H), 1.38 (t, J = 6.9 Hz, 3H), 1.30-1.19 (m, 2H). |
| | | Integration low due to overlap with suppressed water and |
| | | obscurement by DMSO signal. |
| 127 | 546.3 | ¹H NMR (400 MHz, DMSO-d₆) δ 8.64 (d, J = 2.1 Hz, 1H), 8.57 (s, |
| | [M + H]⁺, | 1H), 8.32 (d, J = 2.6 Hz, 1H), 7.99 (s, 1H), 7.75 (dd, J = 8.9, 2.6 Hz, |
| | RT = | 1H), 7.45-7.29 (m, 3H), 7.27 (d, J = 2.0 Hz, 1H), 6.97 (d, J = 8.9 |
| | 1.223 min | Hz, 1H), 4.21-4.07 (m, 4H), 3.28-3.14 (m, 2H), 2.83 (s, 2H), |
| | (UHPLC | 2.39-2.29 (m, 5H), 1.67-1.53 (m, 2H), 1.38 (t, J = 7.0 Hz, 3H). |
| | Method D) | A proton appears to be buried by water peak. |
| 128 | 898.4 | ¹H NMR (500 MHz, DMSO-d₆) δ 10.94 (s, 1H), 8.61 (d, J = 1.8 |
| | [M + H]⁺, | Hz, 1H), 8.54 (s, 1H), 8.30 (d, J = 2.5 Hz, 1H), 7.82-7.74 (m, 1H), |
| | RT = 1.16 | 7.62-7.55 (m, 1H), 7.24 (d, J = 1.6 Hz, 1H), 7.17-7.11 (m, 2H), |
| | min (B) | 6.99 (br d, J = 9.1 Hz, 1H), 5.02 (br dd, J = 13.3, 5.0 Hz, 1H), |
| | | 4.39-4.20 (m, 2H), 4.18-4.10 (m, 2H), 4.04-3.93 (m, 2H), 3.62-3.51 |
| | | (m, 1H), 3.40-3.27 (m, 2H), 3.19-3.06 (m, 4H), 2.93-2.82 (m, |
| | | 1H), 2.65-2.54 (m, 15H), 2.41-2.29 (m, 3H), 2.02-1.93 (m, |
| | | 1H), 1.72-1.45 (m, 9H), 1.37 (t, J = 6.9 Hz, 3H), 1.34-1.28 (m, |
| | | 4H), 1.22 (s, 3H) |
| 129 | 841.3 | ¹H NMR (500 MHz, DMSO-d₆) δ 10.98 (s, 1H), 8.65 (s, 1H), |
| | [M + H]⁺, | 8.57 (s, 1H), 8.33 (d, J = 2.3 Hz, 1H), 7.82 (dd, J = 8.5, 2.1 Hz, 1H), |
| | RT = 1.26 | 7.74-7.69 (m, 1H), 7.48 (s, 1H), 7.41 (br d, J = 8.0 Hz, 1H), 7.27 |
| | min (B) | (d, J = 1.4 Hz, 1H), 7.03 (s, 1H), 5.10 (dd, J = 13.0, 5.0 Hz, 1H), |
| | | 4.49-4.28 (m, 2H), 4.15 (q, J = 7.0 Hz, 2H), 4.06-3.98 (m, 2H), |
| | | 3.68-3.61 (m, 1H), 3.40-3.32 (m, 2H), 3.21-3.06 (m, 3H), 3.03- |
| | | 2.86 (m, 4H), 2.65-2.52 (m, 6H), 2.45-2.33 (m, 1H), 2.10-1.89 |
| | | (m, 5H), 1.69-1.55 (m, 4H), 1.38 (t, J = 6.9 Hz, 3H), 1.24 (s, |
| | | 3H). Integration low due to overlap with suppressed water and |
| | | obscurement by DMSO signal. |
| 130 | 912.4 | ¹H NMR (500 MHz, DMSO-d₆) δ 10.94 (s, 1H), 8.61 (d, J = 1.9 |
| | [M + H]⁺, | Hz, 1H), 8.56-8.50 (m, 1H), 8.30 (d, J = 2.4 Hz, 1H), 7.77 (dd, |
| | RT = 1.19 | J = 8.9, 2.5 Hz, 1H), 7.62-7.57 (m, 1H), 7.24 (d, J = 2.0 Hz, 1H), |
| | min (B) | 7.17-7.12 (m, 2H), 7.00-6.93 (m, 1H), 5.06-4.99 (m, 1H), 4.39- |
| | | 4.21 (m, 2H), 4.14 (q, J = 6.8 Hz, 2H), 3.89-3.80 (m, 2H), 3.56- |
| | | 3.39 (m, 2H), 3.17-3.07 (m, 4H), 2.93-2.84 (m, 1H), 2.63-2.54 |
| | | (m, 12H), 2.41-2.30 (m, 3H), 2.01-1.94 (m, 1H), 1.71-1.57 |
| | | (m, 8H), 1.53-1.46 (m, 2H), 1.39-1.22 (m, 12H), 0.86 (br t, |
| | | J = 7.6 Hz, 3H) |
| 131 | 855.3 | ¹H NMR (500 MHz, DMSO-d₆) δ 10.97 (s, 1H), 8.61 (d, J = 2.1 |
| | [M + H]⁺, | Hz, 1H), 8.54 (s, 1H), 8.30 (d, J = 2.4 Hz, 1H), 7.79 (dd, J = 9.0, 2.6 |
| | RT = 1.24 | Hz, 1H), 7.71 (d, J = 7.8 Hz, 1H), 7.48 (s, 1H), 7.41 (br d, J = 8.2 |
| | min (B) | Hz, 1H), 7.25 (d, J = 2.1 Hz, 1H), 7.01-6.95 (m, 1H), 5.10-5.05 |
| | | (m, 1H), 4.47-4.28 (m, 2H), 4.14 (q, J = 7.0 Hz, 2H), 3.89-3.82 |
| | | (m, 2H), 3.67-3.51 (m, 2H), 3.49-3.30 (m, 2H), 3.18-3.06 (m, |
| | | 4H), 3.03-2.83 (m, 4H), 2.65-2.54 (m, 10H), 2.45-2.32 (m, |
| | | 2H), 2.09-1.89 (m, 5H), 1.68-1.59 (m, 6H), 1.37 (t, J = 7.0 Hz, |
| | | 3H), 0.87 (br t, J = 7.5 Hz, 3H) |
| 132 | 855.4 | ¹H NMR (500 MHz, DMSO-d₆) δ 10.97 (s, 1H), 8.61 (d, J = 2.0 |
| | [M + H]⁺, | Hz, 1H), 8.54 (s, 1H), 8.31 (d, J = 2.4 Hz, 1H), 7.79 (dd, J = 9.0, 2.5 |
| | RT = 1.24 | Hz, 1H), 7.63-7.56 (m, 2H), 7.55-7.48 (m, 1H), 7.25 (d, J = 2.1 |
| | min (B) | Hz, 1H), 6.98 (d, J = 9.2 Hz, 1H), 5.10-5.03 (m, 1H), 4.46-4.28 |
| | | (m, 2H), 4.14 (q, J = 7.1 Hz, 2H), 3.90-3.82 (m, 2H), 3.68-3.30 |
| | | (m, 4H), 3.21-3.03 (m, 4H), 3.03-2.84 (m, 4H), 2.65-2.54 (m, |
| | | 11H), 2.46-2.33 (m, 1H), 2.09-1.88 (m, 5H), 1.69-1.59 (m, |
| | | 6H), 1.37 (t, J = 7.0 Hz, 3H), 0.87 (br t, J = 7.6 Hz, 3H) |
| 133 | 774.2 | ¹H NMR (500 MHz, DMSO-d₆) δ 10.96 (s, 1H), 8.64 (d, J = 2.0 |
| | [M + H]⁺, | Hz, 1H), 8.56 (s, 1H), 8.35-8.29 (m, 1H), 7.80 (dd, J = 8.9, 2.4 |
| | RT = 1.28 | Hz, 1H), 7.65 (d, J = 8.5 Hz, 1H), 7.26 (d, J = 2.1 Hz, 1H), 7.16 (s, |
| | min (B) | 1H), 7.07 (dd, J = 8.5, 2.1 Hz, 1H), 7.00 (d, J = 9.0 Hz, 1H), 5.06 |
| | | (dd, J = 13.3, 5.1 Hz, 1H), 5.01 (br s, 2H), 4.43-4.24 (m, 2H), |
| | | 4.15 (q, J = 6.9 Hz, 2H), 3.91-3.83 (m, 2H), 3.23-3.13 (m, 1H), |
| | | 2.95-2.85 (m, 1H), 2.64-2.54 (m, 12H), 2.45-2.32 (m, 1H), |
| | | 2.03-1.95 (m, 1H), 1.71-1.60 (m, 6H), 1.38 (t, J = 7.0 Hz, 3H), |
| | | 0.88 (br t, J = 7.4 Hz, 3H) |
| 134 | 925.3 | ¹H NMR (500 MHz, DMSO-d₆) δ 10.93 (s, 1H), 8.93 (s, 1H), |
| | [M + H]⁺, | 8.66-8.61 (m, 1H), 8.58-8.53 (m, 1H), 8.35-8.31 (m, 1H), 7.81 |
| | RT = 1.94 | (dd, J = 8.7, 2.7 Hz, 1H), 7.50 (d, J = 9.1 Hz, 1H), 7.46-7.36 (m, |
| | min (B) | 3H), 7.30-7.27 (m, 1H), 7.06-7.04 (m, 3H), 5.07-4.97 (m, 1H), |
| | | 4.35-4.28 (m, 1H), 4.22-4.12 (m, 3H), 4.11-4.02 (m, 2H), 3.51- |
| | | 3.39 (m, 8H), 3.36-3.27 (m, 4H), 2.43-2.27 (m, 2H), 2.26- |
| | | 2.04 (m, 4H), 1.60-1.52 (m, 4H), 1.46-1.34 (m, 7H) |
| 135 | 1053.4 | ¹H NMR (400 MHz, DMSO-d₆) δ 8.64 (d, J = 2.0 Hz, 1H), 8.57 (s, |
| (NMe) | [M + H]⁺, | 1H), 8.32 (d, J = 2.9 Hz, 1H), 8.12 (s, 1H), 7.75 (dd, J = 8.8, 2.6 Hz, |
| | RT = | 1H), 7.52 (d, J = 9.3 Hz, 1H), 7.38-7.31 (m, 3H), 7.27 (d, J = 2.0 |
| | 1.301 min | Hz, 1H), 7.08-7.02 (m, 2H), 6.97 (d, J = 8.7 Hz, 1H), 5.11 (dd, |
| | (UHPLC | J = 13.4, 5.1 Hz, 1H), 4.35-4.28 (m, 1H), 4.24-4.12 (m, 5H), |
| | Method D) | 3.40 (br d, J = 2.6 Hz, 4H), 3.28 (br d, J = 2.9 Hz, 4H), 3.16 (br t, |
| | | J = 12.1 Hz, 2H), 2.99 (s, 3H), 2.98-2.91 (m, 1H), 2.79-2.66 (m, |
| | | 3H), 2.49-2.20 (m, 12H), 2.02-1.92 (m, 1H), 1.68-1.55 (m, |
| | | 2H), 1.54-1.41 (m, 4H), 1.38 (t, J = 7.0 Hz, 3H), 1.33-1.26 (m, |
| | | 4H). Integration low due to obscurement by DMSO signal. |

Examples 136-171 were prepared using procedures similar to the ones described in the aforementioned examples.

| | | | Analytical |
|---|---|---|---|
| | | | LCMS |
| | | | ESI-MS(+) |
| Ex- | | | m/z/RT |
| ample | | | (min) |
| # | Structure | Name | (Method); |
| Ex- ample 136 | 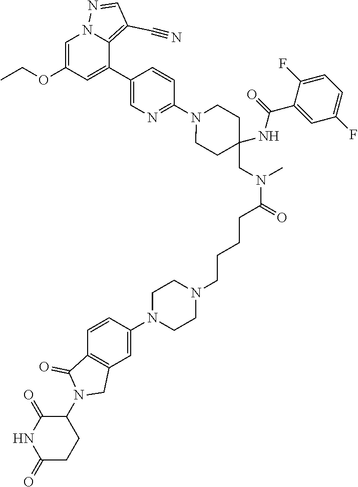 [SMILES: CCOc1cc(-c2ccc(N3CCC(CN(C)C(=O)CCCCN4CCN(c5ccc6c(c5)CN(C5CCC(=O)NC5=O)C6=O)CC4)(NC(=O)c4cc(F)ccc4F)CC3)nc2)c2c(C#N)cnn2c1] | N-[1-(5-{3-cyano-6- ethoxypyrazolo[1,5- a]pyridin-4-yl}pyridin-2-yl)- 4-[(5-{4-[2-(2,6- dioxopiperidin-3-yl)-1-oxo- 2,3-dihydro-1H-isoindol-5- yl]piperazin-1-yl}-N- methylpentanamido)methyl] piperidin-4-yl]-2,5- difluorobenzamide | 956 [M + H]⁺, RT = 1.79 min (A) |
| Ex- ample 137 | 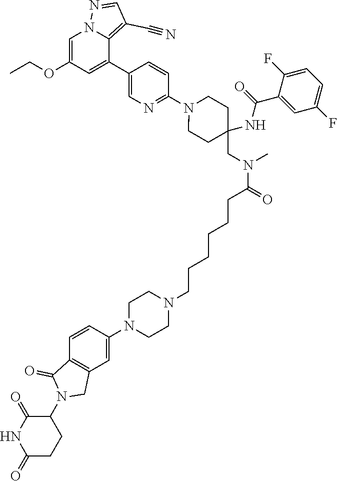 [SMILES: CCOc1cc(-c2ccc(N3CCC(CN(C)C(=O)CCCCCCN4CCN(c5ccc6c(c5)CN(C5CCC(=O)NC5=O)C6=O)CC4)(NC(=O)c4cc(F)ccc4F)CC3)nc2)c2c(C#N)cnn2c1] | N-[1-(5-{3-cyano-6- ethoxypyrazolo[1,5- a]pyridin-4-yl}pyridin-2-yl)- 4-[(7-{4-[2-(2,6- dioxopiperidin-3-yl)-1-oxo- 2,3-dihydro-1H-isoindol-5- yl]piperazin-1-yl}-N- methylheptanamido)methyl] piperidin-4-yl]-2,5- difluorobenzamide | 984 [M + H]⁺, RT = 1.99 min (A) |
| Ex- ample 138 | 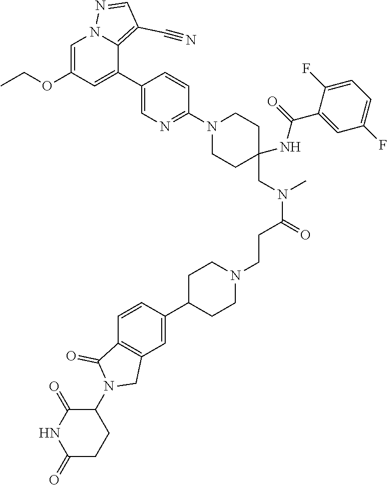 [SMILES: CCOc1cc(-c2ccc(N3CCC(CN(C)C(=O)CCN4CCC(c5ccc6c(c5)CN(C5CCC(=O)NC5=O)C6=O)CC4)(NC(=O)c4cc(F)ccc4F)CC3)nc2)c2c(C#N)cnn2c1] | N-[1-(5-{3-cyano-6- ethoxypyrazolo[1,5- a]pyridin-4-yl}pyridin-2-yl)- 4-[(3-{4-[2-(2,6- dioxopiperidin-3-yl)-1-oxo- 2,3-dihydro-1H-isoindol-5- yl]piperidin-1-yl}-N- methylpropanamido)methyl] piperidin-4-yl]-2,5- difluorobenzamide | 927 [M + H]⁺, RT = 1.96 min (A) |
| Ex- ample 139 | 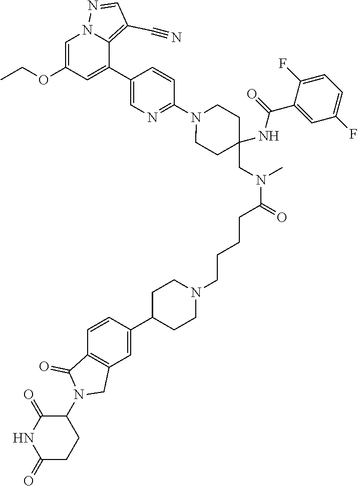 [SMILES: CCOc1cc(-c2ccc(N3CCC(CN(C)C(=O)CCCCN4CCC(c5ccc6c(c5)CN(C5CCC(=O)NC5=O)C6=O)CC4)(NC(=O)c4cc(F)ccc4F)CC3)nc2)c2c(C#N)cnn2c1] | N-[1-(5-{3-cyano-6- ethoxypyrazolo[1,5- a]pyridin-4-yl}pyridin-2-yl)- 4-[(5-{4-[2-(2,6- dioxopiperidin-3-yl)-1-oxo- 2,3-dihydro-1H-isoindol-5- yl]piperidin-1-yl}-N- methylpentanamido)methyl] piperidin-4-yl]-2,5- difluorobenzamide | 955 [M + H]⁺, RT = 1.72 min (A) |
| Ex- ample 140 | 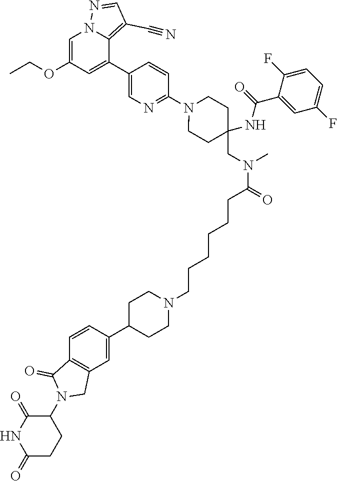 [SMILES: CCOc1cc(-c2ccc(N3CCC(CN(C)C(=O)CCCCCCN4CCC(c5ccc6c(c5)CN(C5CCC(=O)NC5=O)C6=O)CC4)(NC(=O)c4cc(F)ccc4F)CC3)nc2)c2c(C#N)cnn2c1] | N-[1-(5-{3-cyano-6- ethoxypyrazolo[1,5- a]pyridin-4-yl}pyridin-2-yl)- 4-[(7-{4-[2-(2,6- dioxopiperidin-3-yl)-1-oxo- 2,3-dihydro-1H-isoindol-5- yl]piperidin-1-yl}-N- methylheptanamido)methyl] piperidin-4-yl]-2,5- difluorobenzamide | 983 [M + H]⁺, RT = 1.82 min (A) |
| Ex- ample 141 | 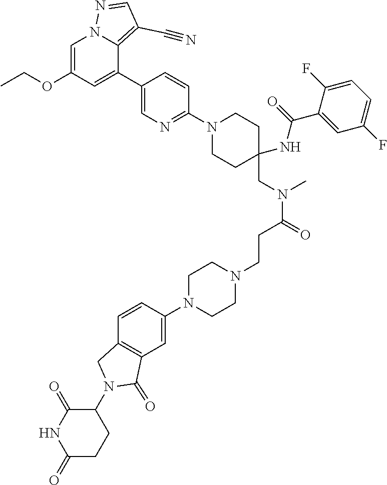 [SMILES: CCOc1cc(-c2ccc(N3CCC(CN(C)C(=O)CCN4CCN(c5ccc6c(c5)C(=O)N(C5CCC(=O)NC5=O)C6)CC4)(NC(=O)c4cc(F)ccc4F)CC3)nc2)c2c(C#N)cnn2c1] | N-[1-(5-{3-cyano-6- ethoxypyrazolo[1,5- a]pyridin-4-yl}pyridin-2-yl)- 4-[(3-{4-[2-(2,6- dioxopiperidin-3-yl)-3-oxo- 2,3-dihydro-1H-isoindol-5- yl]piperazin-1-yl}-N- methylpropanamido)methyl] piperidin-4-yl]-2,5- difluorobenzamide | 928 [M + H]⁺, RT = 1.88 min (A) |
| Ex- ample 142 | 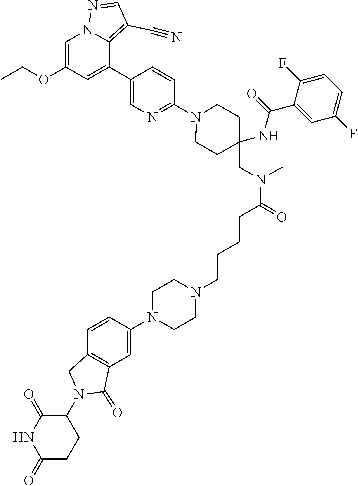 [SMILES: CCOc1cc(-c2ccc(N3CCC(CN(C)C(=O)CCCCN4CCN(c5ccc6c(c5)C(=O)N(C5CCC(=O)NC5=O)C6)CC4)(NC(=O)c4cc(F)ccc4F)CC3)nc2)c2c(C#N)cnn2c1] | N-[1-(5-{3-cyano-6- ethoxypyrazolo[1,5- a]pyridin-4-yl}pyridin-2-yl)- 4-[(5-{4-[2-(2,6- dioxopiperidin-3-yl)-3-oxo- 2,3-dihydro-1H-isoindol-5- yl]piperazin-1-yl}-N- methylpentanamido)methyl] piperidin-4-yl]-2,5- difluorobenzamide | 956 [M + H]⁺, RT = 1.82 min (A) |
| Ex- ample 143 | 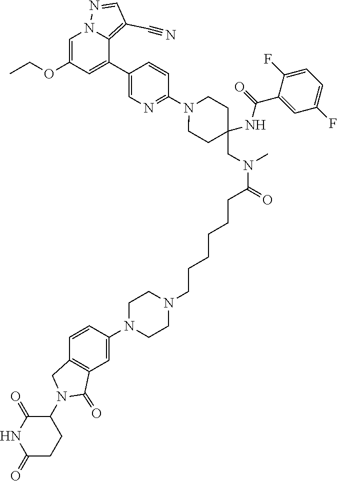 [SMILES: CCOc1cc(-c2ccc(N3CCC(CN(C)C(=O)CCCCCCN4CCN(c5ccc6c(c5)C(=O)N(C5CCC(=O)NC5=O)C6)CC4)(NC(=O)c4cc(F)ccc4F)CC3)nc2)c2c(C#N)cnn2c1] | N-[1-(5-{3-cyano-6- ethoxypyrazolo[1,5- a]pyridin-4-yl}pyridin-2-yl)- 4-[(7-{4-[2-(2,6- dioxopiperidin-3-yl)-3-oxo- 2,3-dihydro-1H-isoindol-5- yl]piperazin-1-yl}-N- methylheptanamido)methyl] piperidin-4-yl]-2,5- difluorobenzamide | 984 [M + H]⁺, RT = 1.88 min (A) |
| Ex- ample 144 | 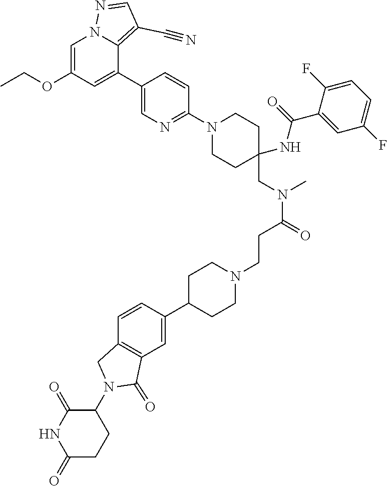 [SMILES: CCOc1cc(-c2ccc(N3CCC(CN(C)C(=O)CCN4CCC(c5ccc6c(c5)C(=O)N(C5CCC(=O)NC5=O)C6)CC4)(NC(=O)c4cc(F)ccc4F)CC3)nc2)c2c(C#N)cnn2c1] | N-[1-(5-{3-cyano-6- ethoxypyrazolo[1,5- a]pyridin-4-yl}pyridin-2-yl)- 4-[(3-{4-[2-(2,6- dioxopiperidin-3-yl)-3-oxo- 2,3-dihydro-1H-isoindol-5- yl]piperidin-1-yl}-N- methylpropanamido)methyl] piperidin-4-yl]-2,5- difluorobenzamide | 927 [M + H]⁺, RT = 1.73 min (A) |
| Ex- ample 145 | 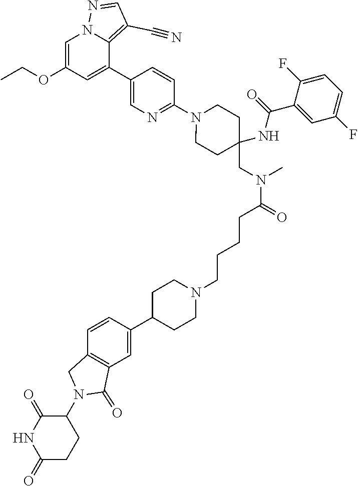 [SMILES: CCOc1cc(-c2ccc(N3CCC(CN(C)C(=O)CCCCN4CCC(c5ccc6c(c5)C(=O)N(C5CCC(=O)NC5=O)C6)CC4)(NC(=O)c4cc(F)ccc4F)CC3)nc2)c2c(C#N)cnn2c1] | N-[1-(5-{3-cyano-6- ethoxypyrazolo[1,5- a]pyridin-4-yl}pyridin-2-yl)- 4-[(5-{4-[2-(2,6- dioxopiperidin-3-yl)-3-oxo- 2,3-dihydro-1H-isoindol-5- yl]piperidin-1-yl}-N- methylpentanamido)methyl] piperidin-4-yl]-2,5- difluorobenzamide | 955 [M + H]⁺, RT = 1.72 min (A) |
| Ex- ample 146 | 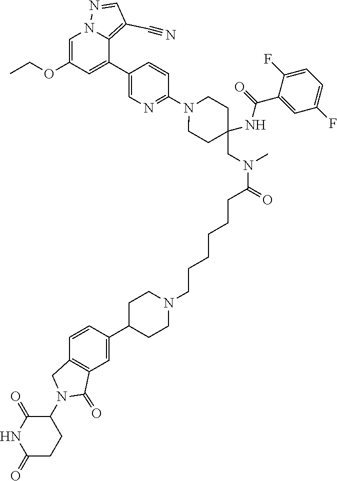 [SMILES: CCOc1cc(-c2ccc(N3CCC(CN(C)C(=O)CCCCCCN4CCC(c5ccc6c(c5)C(=O)N(C5CCC(=O)NC5=O)C6)CC4)(NC(=O)c4cc(F)ccc4F)CC3)nc2)c2c(C#N)cnn2c1] | N-[1-(5-{3-cyano-6- ethoxypyrazolo[1,5- a]pyridin-4-yl}pyridin-2-yl)- 4-[(7-{4-[2-(2,6- dioxopiperidin-3-yl)-3-oxo- 2,3-dihydro-1H-isoindol-5- yl]piperidin-1-yl}-N- methylheptanamido)methyl] piperidin-4-yl]-2,5- difluorobenzamide | 983 [M + H]⁺, RT = 1.83 min (A) |
| Ex- ample 147 | 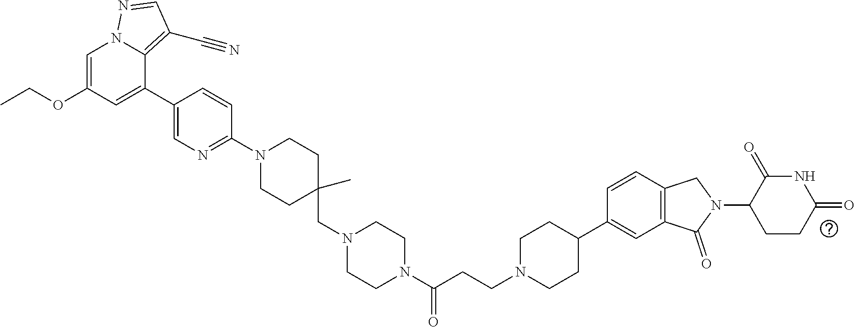 [SMILES: CCOc1cc(-c2ccc(N3CCC(C)(CN4CCN(C(=O)CCN5CCC(c6ccc7c(c6)C(=O)N(C6CCC(=O)NC6=O)C7)CC5)CC4)CC3)nc2)c2c(C#N)cnn2c1] | 4-[6-(4-{[4-(3-{4-[2-(2,6- dioxopiperidin-3-yl)-3-oxo- 2,3-dihydro-1H-isoindol-5- yl]piperidin-1- yl}propanoyl)piperazin-1- yl]methyl}-4- methylpiperidin-1-yl)pyridin- 3-yl]-6-ethoxypyrazolo[1,5- a]pyridine-3-carbonitrile | |
| Ex- ample 148 | 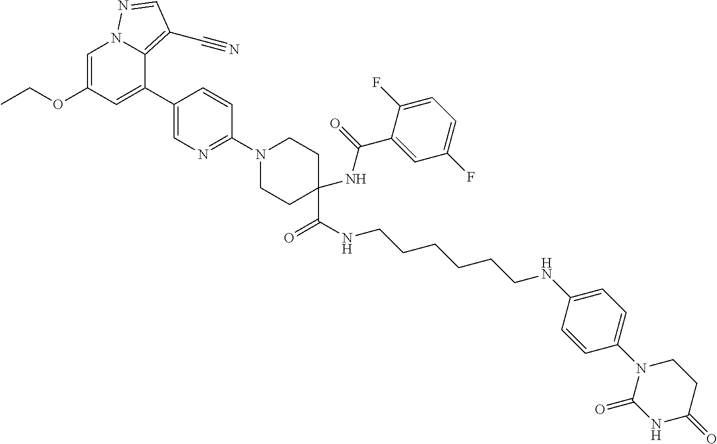 [SMILES: CCOc1cc(-c2ccc(N3CCC(NC(=O)c4cc(F)ccc4F)(C(=O)NCCCCCCNc4ccc(N5CCC(=O)NC5=O)cc4)CC3)nc2)c2c(C#N)cnn2c1] | 1-(5-{3-cyano-6- ethoxypyrazolo[1,5- a]pyridin-4-yl}pyridin-2-yl)- 4-(2,5-difluorobenzamido)-N- (6-{[4-(2,4-dioxo-1,3- diazinan-1- yl)phenyl]amino}hexyl) piperidine-4-carboxamide | 834 [M + H]⁺, RT = 1.98 min (A) |
| Ex- ample 149 | 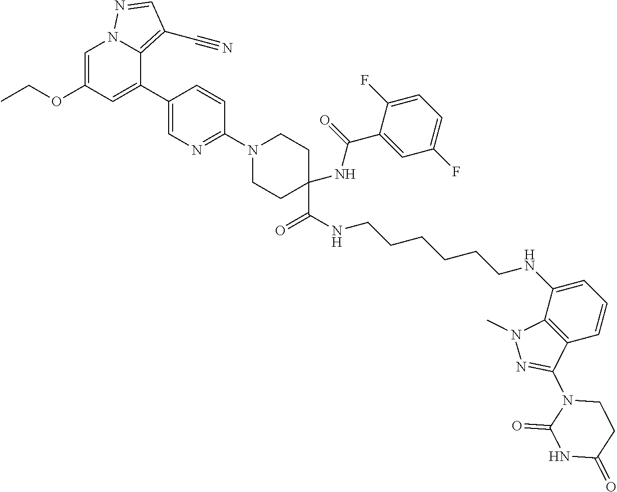 [SMILES: CCOc1cc(-c2ccc(N3CCC(NC(=O)c4cc(F)ccc4F)(C(=O)NCCCCCCNc4cccc5c(N6CCC(=O)NC6=O)nn(C)c45)CC3)nc2)c2c(C#N)cnn2c1] | 1-(5-{3-cyano-6- ethoxypyrazolo[1,5- a]pyridin-4-yl}pyridin-2-yl)- 4-(2,5-difluorobenzamido)-N- (6-{[3-(2,4-dioxo-1,3- diazinan-1-yl)-1-methyl-1H- indazol-7- yl]amino}hexyl)piperidine-4- carboxamide | 887 [M + H]⁺, RT = 2.08 min (A) |
| Ex- ample 150 | 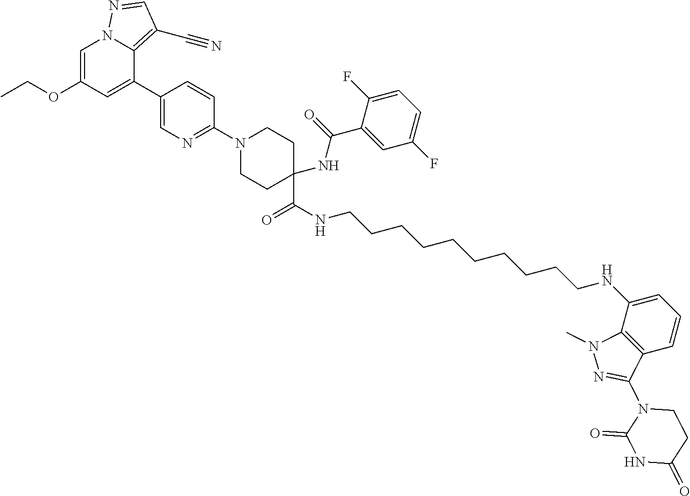 [SMILES: CCOc1cc(-c2ccc(N3CCC(NC(=O)c4cc(F)ccc4F)(C(=O)NCCCCCCCCCCNc4cccc5c(N6CCC(=O)NC6=O)nn(C)c45)CC3)nc2)c2c(C#N)cnn2c1] | 1-(5-{3-cyano-6- ethoxypyrazolo[1,5- a]pyridin-4-yl}pyridin-2-yl)- 4-(2,5-difluorobenzamido)-N- (10-{[3-(2,4-dioxo-1,3- diazinan-1-yl)-1-methyl-1H- indazol-7- yl]amino}decyl)piperidine-4- carboxamide | 943 [M + H]⁺, RT = 2.33 min (A) |
| Ex- ample 151 | 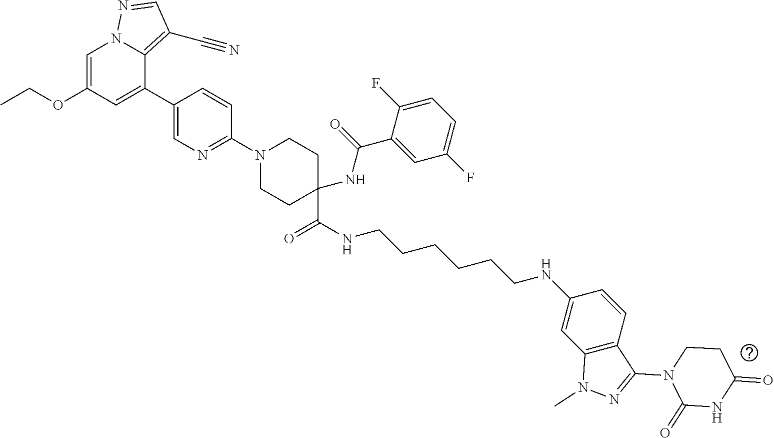 [SMILES: CCOc1cc(-c2ccc(N3CCC(NC(=O)c4cc(F)ccc4F)(C(=O)NCCCCCCNc4ccc5c(N6CCC(=O)NC6=O)nn(C)c5c4)CC3)nc2)c2c(C#N)cnn2c1] | 1-(5-{3-cyano-6- ethoxypyrazolo[1,5- a]pyridin-4-yl}pyridin-2-yl)- 4-(2,5-difluorobenzamido)-N- (6-{[3-(2,4-dioxo-1,3- diazinan-1-yl)-1-methyl-1H- indazol-6- yl]amino}hexyl)piperidine-4- carboxamide | 887 [M + H]⁺, RT = 2.02 min (A) |
| Ex- ample 152 | 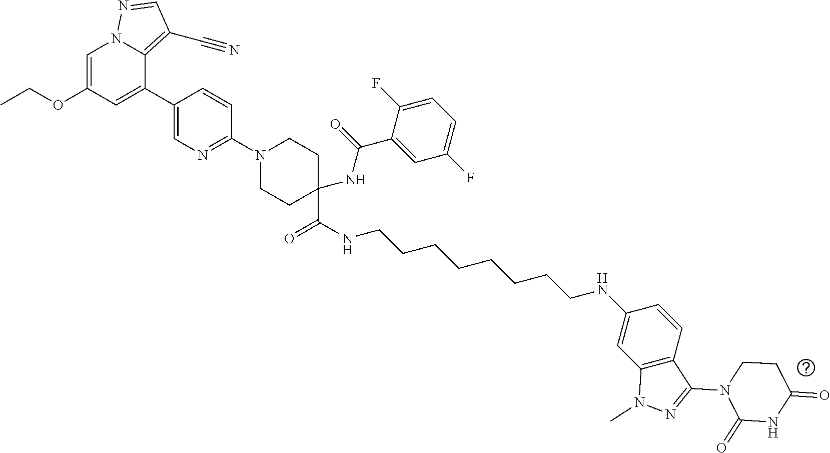 [SMILES: CCOc1cc(-c2ccc(N3CCC(NC(=O)c4cc(F)ccc4F)(C(=O)NCCCCCCCCNc4ccc5c(N6CCC(=O)NC6=O)nn(C)c5c4)CC3)nc2)c2c(C#N)cnn2c1] | 1-(5-{3-cyano-6- ethoxypyrazolo[1,5- a]pyridin-4-yl}pyridin-2-yl)- 4-(2,5-difluorobenzamido)-N- (8-{[3-(2,4-dioxo-1,3- diazinan-1-yl)-1-methyl-1H- indazol-6- yl]amino}octyl)piperidine-4- carboxamide | 915 [M + H]⁺, RT = 2.15 min (A) |
| Ex- ample 153 | 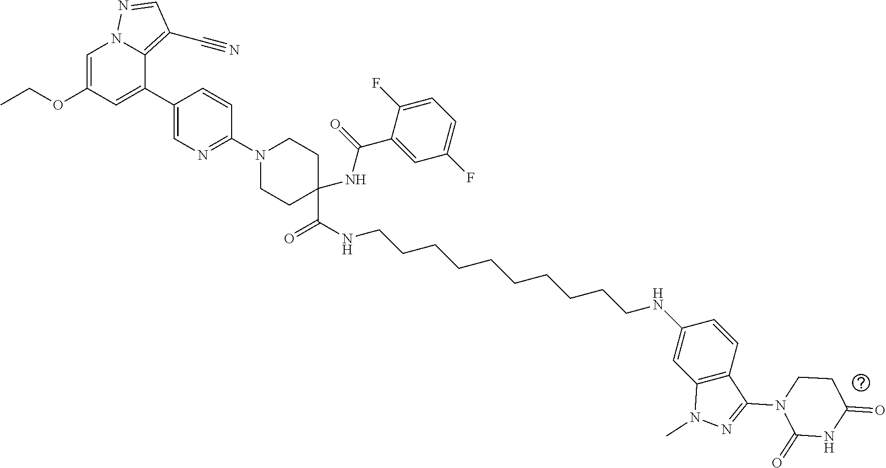 [SMILES: CCOc1cc(-c2ccc(N3CCC(NC(=O)c4cc(F)ccc4F)(C(=O)NCCCCCCCCCCNc4ccc5c(N6CCC(=O)NC6=O)nn(C)c5c4)CC3)nc2)c2c(C#N)cnn2c1] | 1-(5-{3-cyano-6- ethoxypyrazolo[1,5- a]pyridin-4-yl}pyridin-2-yl)- 4-(2,5-difluorobenzamido)-N- (10-{[3-(2,4-dioxo-1,3- diazinan-1-yl)-1-methyl-1H- indazol-6- yl]amino}decyl)piperidine-4- carboxamide | 943 [M + H]⁺, RT = 2.33 min (A) |
| Ex- ample 154 | 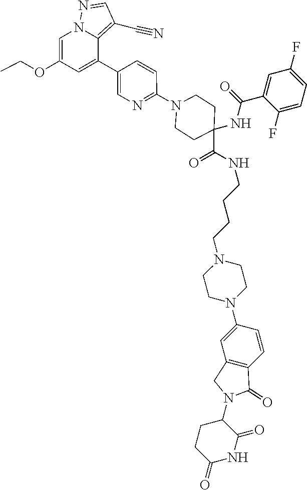 [SMILES: CCOc1cc(-c2ccc(N3CCC(NC(=O)c4cc(F)ccc4F)(C(=O)NCCCCN4CCN(c5ccc6c(c5)CN(C5CCC(=O)NC5=O)C6=O)CC4)CC3)nc2)c2c(C#N)cnn2c1] | 1-(5-{3-cyano-6- ethoxypyrazolo[1,5- a]pyridin-4-yl}pyridin-2-yl)- 4-(2,5-difluorobenzamido)-N- (4-{4-[2-(2,6-dioxopiperidin- 3-yl)-1-oxo-2,3-dihydro-1H- isoindol-5-yl]piperazin-1- yl}butyl)piperidine-4- carboxamide | 928 [M + H]⁺, RT = 1.76 min (A) |
| Ex- ample 155 | 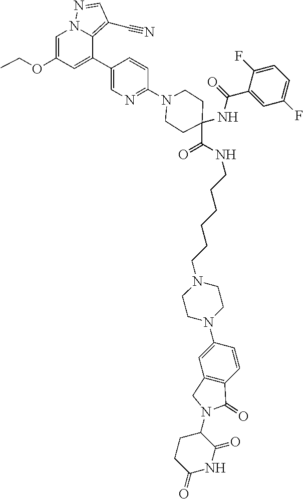 [SMILES: CCOc1cc(-c2ccc(N3CCC(NC(=O)c4cc(F)ccc4F)(C(=O)NCCCCCCN4CCN(c5ccc6c(c5)CN(C5CCC(=O)NC5=O)C6=O)CC4)CC3)nc2)c2c(C#N)cnn2c1] | 1-(5-{3-cyano-6- ethoxypyrazolo[1,5- a]pyridin-4-yl}pyridin-2-yl)- 4-(2,5-difluorobenzamido)-N- (6-{4-[2-(2,6-dioxopiperidin- 3-yl)-1-oxo-2,3-dihydro-1H- isoindol-5-yl]piperazin-1- yl}hexyl)piperidine-4- carboxamide | 956 [M + H]⁺, RT = 1.85 min (A) |
| Ex- ample 156 | 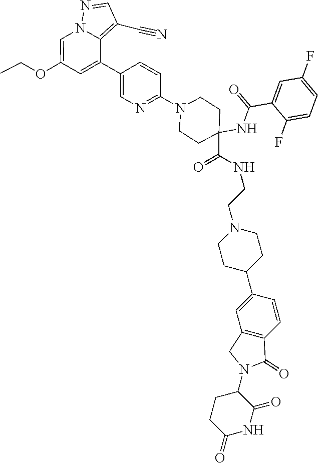 [SMILES: CCOc1cc(-c2ccc(N3CCC(NC(=O)c4cc(F)ccc4F)(C(=O)NCCN4CCC(c5ccc6c(c5)CN(C5CCC(=O)NC5=O)C6=O)CC4)CC3)nc2)c2c(C#N)cnn2c1] | 1-(5-{3-cyano-6- ethoxypyrazolo[1,5- a]pyridin-4-yl}pyridin-2-yl)- 4-(2,5-difluorobenzamido)-N- (2-{4-[2-(2,6-dioxopiperidin- 3-yl)-1-oxo-2,3-dihydro-1H- isoindol-5-yl]piperidin-1- yl}ethyl)piperidine-4- carboxamide | 899 [M + H]⁺, RT = 1.78 min (A) |
| Ex- ample 157 | 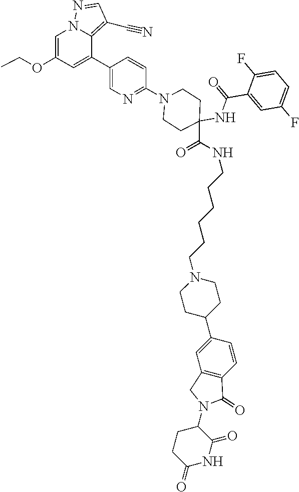 [SMILES: CCOc1cc(-c2ccc(N3CCC(NC(=O)c4cc(F)ccc4F)(C(=O)NCCCCCCN4CCC(c5ccc6c(c5)CN(C5CCC(=O)NC5=O)C6=O)CC4)CC3)nc2)c2c(C#N)cnn2c1] | 1-(5-{3-cyano-6- ethoxypyrazolo[1,5- a]pyridin-4-yl}pyridin-2-yl)- 4-(2,5-difluorobenzamido)-N- (6-{4-[2-(2,6-dioxopiperidin- 3-yl)-1-oxo-2,3-dihydro-1H- isoindol-5-yl]piperidin-1- yl}hexyl)piperidine-4- carboxamide | 955 [M + H]⁺, RT = 1.55 min (B) |
| Ex- ample 158 | 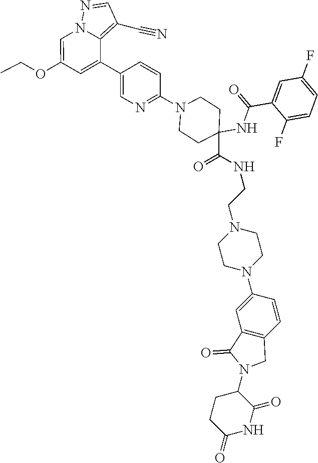 [SMILES: CCOc1cc(-c2ccc(N3CCC(NC(=O)c4cc(F)ccc4F)(C(=O)NCCN4CCN(c5ccc6c(c5)C(=O)N(C5CCC(=O)NC5=O)C6)CC4)CC3)nc2)c2c(C#N)cnn2c1] | 1-(5-{3-cyano-6- ethoxypyrazolo[1,5- a]pyridin-4-yl}pyridin-2-yl)- 4-(2,5-difluorobenzamido)-N- (2-{4-[2-(2,6-dioxopiperidin- 3-yl)-3-oxo-2,3-dihydro-1H- isoindol-5-yl]piperazin-1- yl}ethyl)piperidine-4- carboxamide | 900 [M + H]⁺, RT = 1.76 min (A) |
| Ex- ample 159 | 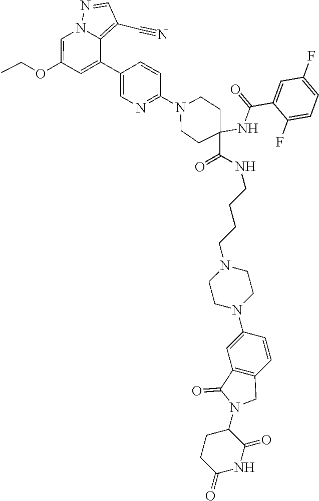 [SMILES: CCOc1cc(-c2ccc(N3CCC(NC(=O)c4cc(F)ccc4F)(C(=O)NCCCCN4CCN(c5ccc6c(c5)C(=O)N(C5CCC(=O)NC5=O)C6)CC4)CC3)nc2)c2c(C#N)cnn2c1] | 1-(5-{3-cyano-6- ethoxypyrazolo[1,5- a]pyridin-4-yl}pyridin-2-yl)- 4-(2,5-difluorobenzamido)-N- (4-{4-[2-(2,6-dioxopiperidin- 3-yl)-3-oxo-2,3-dihydro-1H- isoindol-5-yl]piperazin-1- yl}butyl)piperidine-4- carboxamide | 928.4 [M + H]⁺, RT = 1.5 min (B) |
| Ex- ample 160 | 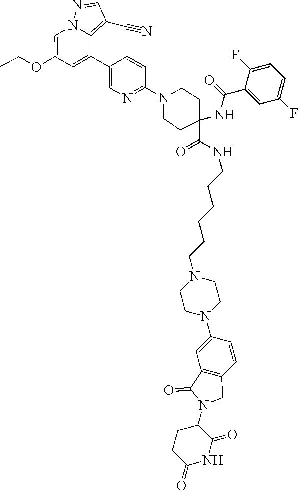 [SMILES: CCOc1cc(-c2ccc(N3CCC(NC(=O)c4cc(F)ccc4F)(C(=O)NCCCCCCN4CCN(c5ccc6c(c5)C(=O)N(C5CCC(=O)NC5=O)C6)CC4)CC3)nc2)c2c(C#N)cnn2c1] | 1-(5-{3-cyano-6- ethoxypyrazolo[1,5- a]pyridin-4-yl}pyridin-2-yl)- 4-(2,5-difluorobenzamido)-N- (6-{4-[2-(2,6-dioxopiperidin- 3-yl)-3-oxo-2,3-dihydro-1H- isoindol-5-yl]piperazin-1- yl}hexyl)piperidine-4- carboxamide | 956 [M + H]⁺, RT = 1.39 min (B) |
| Ex- ample 161 | 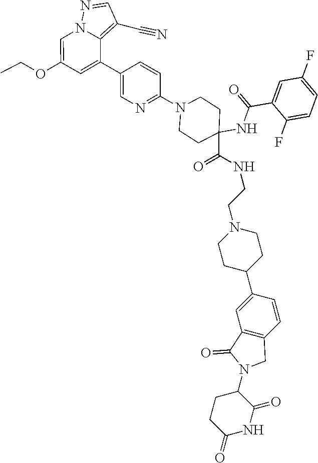 [SMILES: CCOc1cc(-c2ccc(N3CCC(NC(=O)c4cc(F)ccc4F)(C(=O)NCCN4CCC(c5ccc6c(c5)C(=O)N(C5CCC(=O)NC5=O)C6)CC4)CC3)nc2)c2c(C#N)cnn2c1] | 1-(5-{3-cyano-6- ethoxypyrazolo[1,5- a]pyridin-4-yl}pyridin-2-yl)- 4-(2,5-difluorobenzamido)-N- (2-{4-[2-(2,6-dioxopiperidin- 3-yl)-3-oxo-2,3-dihydro-1H- isoindol-5-yl]piperidin-1- yl}ethyl)piperidine-4- carboxamide | 899 [M + H]⁺, RT = 1.68 min (A) |
| Ex- ample 162 | 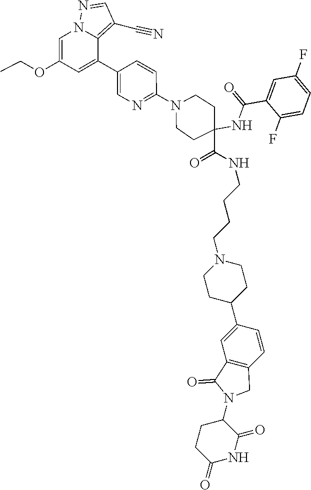 [SMILES: CCOc1cc(-c2ccc(N3CCC(NC(=O)c4cc(F)ccc4F)(C(=O)NCCCCN4CCC(c5ccc6c(c5)C(=O)N(C5CCC(=O)NC5=O)C6)CC4)CC3)nc2)c2c(C#N)cnn2c1] | 1-(5-{3-cyano-6- ethoxypyrazolo[1,5- a]pyridin-4-yl}pyridin-2-yl)- 4-(2,5-difluorobenzamido)-N- (4-{4-[2-(2,6-dioxopiperidin- 3-yl)-3-oxo-2,3-dihydro-1H- isoindol-5-yl]piperidin-1- yl}butyl)piperidine-4- carboxamide | 927 [M + H]⁺, RT = 1.75 min (A) |
| Ex- ample 163 | 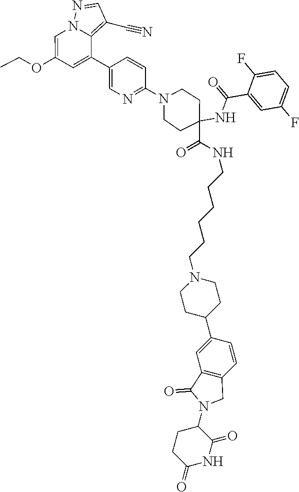 [SMILES: CCOc1cc(-c2ccc(N3CCC(NC(=O)c4cc(F)ccc4F)(C(=O)NCCCCCCN4CCC(c5ccc6c(c5)C(=O)N(C5CCC(=O)NC5=O)C6)CC4)CC3)nc2)c2c(C#N)cnn2c1] | 1-(5-{3-cyano-6- ethoxypyrazolo[1,5- a]pyridin-4-yl}pyridin-2-yl)- 4-(2,5-difluorobenzamido)-N- (6-{4-[2-(2,6-dioxopiperidin- 3-yl)-3-oxo-2,3-dihydro-1H- isoindol-5-yl]piperidin-1- yl}hexyl)piperidine-4- carboxamide | 955 [M + H]⁺, RT = 1.76 min (A) |
| Ex- ample 164 | 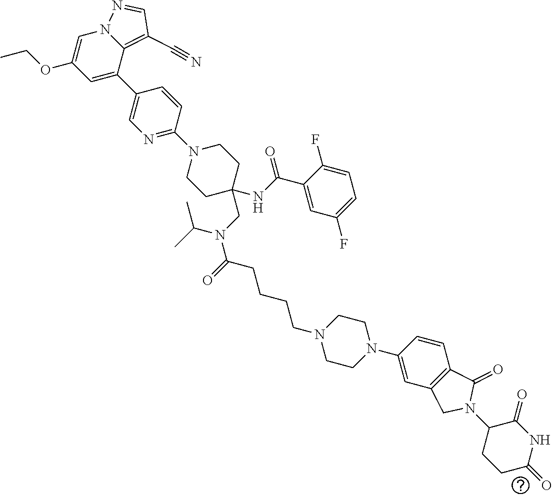 [SMILES: CCOc1cc(-c2ccc(N3CCC(CN(C(=O)CCCCN4CCN(c5ccc6c(c5)CN(C5CCC(=O)NC5=O)C6=O)CC4)C(C)C)(NC(=O)c4cc(F)ccc4F)CC3)nc2)c2c(C#N)cnn2c1] | N-[1-(5-{3-cyano-6- ethoxypyrazolo[1,5- a]pyridin-4-yl}pyridin-2-yl)- 4-[(5-{4-[2-(2,6- dioxopiperidin-3-yl)-1-oxo- 2,3-dihydro-1H-isoindol-5- yl]piperazin-1-yl}-N- (propan-2- yl)pentanamido)methyl] piperidin-4-yl]-2,5- difluorobenzamide | 984 [M + H]⁺, RT = 1.99 min (A) |
| Ex- ample 165 | 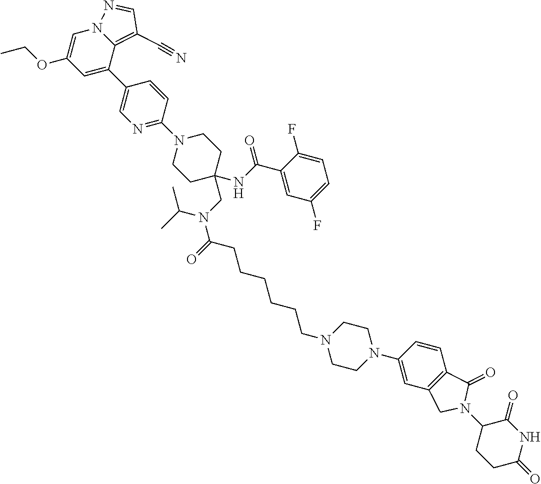 [SMILES: CCOc1cc(-c2ccc(N3CCC(CN(C(=O)CCCCCCN4CCN(c5ccc6c(c5)CN(C5CCC(=O)NC5=O)C6=O)CC4)C(C)C)(NC(=O)c4cc(F)ccc4F)CC3)nc2)c2c(C#N)cnn2c1] | N-[1-(5-{3-cyano-6- ethoxypyrazolo[1,5- a]pyridin-4-yl}pyridin-2-yl)- 4-[(7-{4-[2-(2,6- dioxopiperidin-3-yl)-1-oxo- 2,3-dihydro-1H-isoindol-5- yl]piperazin-1-yl}-N- (propan-2- yl)heptanamido)methyl] piperidin-4-yl]-2,5- difluorobenzamide | 1012 [M + H]⁺, RT = 2.18 min (A) |
| Ex- ample 166 | 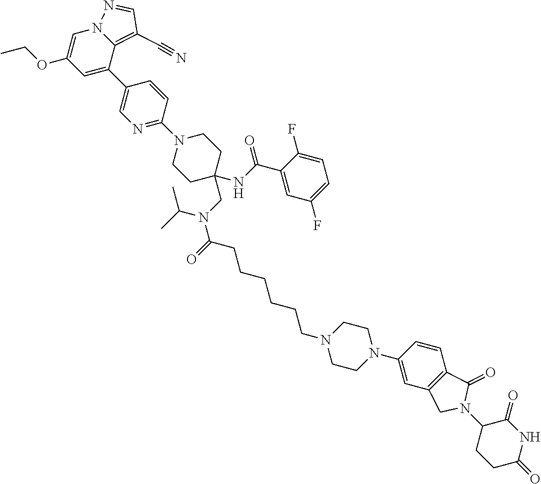 [SMILES: CCOc1cc(-c2ccc(N3CCC(CN(C(=O)CCCCCCN4CCN(c5ccc6c(c5)CN(C5CCC(=O)NC5=O)C6=O)CC4)C(C)C)(NC(=O)c4cc(F)ccc4F)CC3)nc2)c2c(C#N)cnn2c1] | N-[1-(5-{3-cyano-6- ethoxypyrazolo[1,5- a]pyridin-4-yl}pyridin-2-yl)- 4-[(7-{4-[2-(2,6- dioxopiperidin-3-yl)-1-oxo- 2,3-dihydro-1H-isoindol-5- yl]piperidin-1-yl}-N- (propan-2- yl)heptanamido)methyl] piperidin-4-yl]-2,5- difluorobenzamide | 1011 [M + H]⁺, RT = 2.10 min (A) |
| Ex- ample 167 | 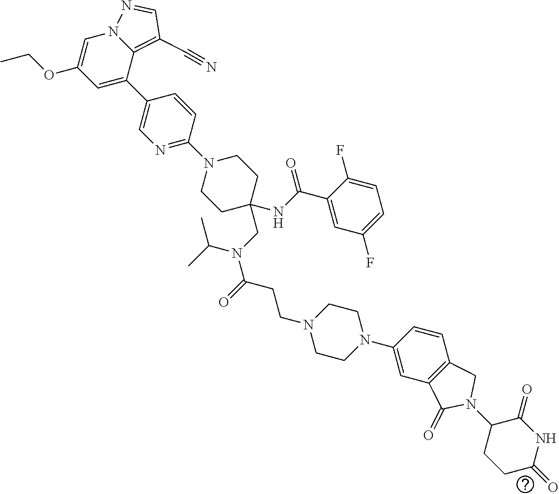 [SMILES: CCOc1cc(-c2ccc(N3CCC(CN(C(=O)CCN4CCN(c5ccc6c(c5)C(=O)N(C5CCC(=O)NC5=O)C6)CC4)C(C)C)(NC(=O)c4cc(F)ccc4F)CC3)nc2)c2c(C#N)cnn2c1] | N-[1-(5-{3-cyano-6- ethoxypyrazolo[1,5- a]pyridin-4-yl}pyridin-2-yl)- 4-[(3-{4-[2-(2,6- dioxopiperidin-3-yl)-3-oxo- 2,3-dihydro-1H-isoindol-5- yl]piperazin-1-yl}-N- (propan-2- yl)propanamido)methyl] piperidin-4-yl]-2,5- difluorobenzamide | 956 [M + H]⁺, RT = 2.03 min (A) |
| Ex- ample 168 | 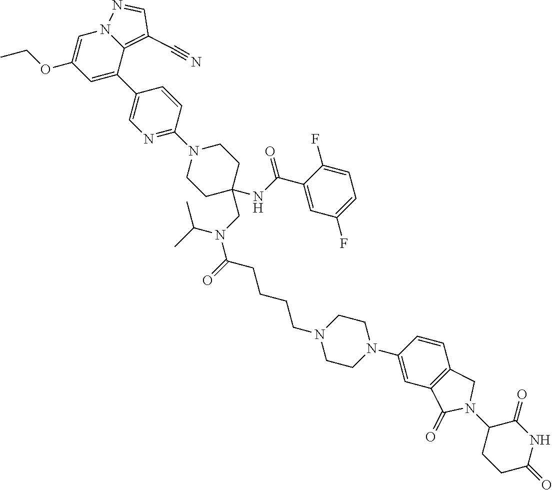 [SMILES: CCOc1cc(-c2ccc(N3CCC(CN(C(=O)CCCCN4CCN(c5ccc6c(c5)C(=O)N(C5CCC(=O)NC5=O)C6)CC4)C(C)C)(NC(=O)c4cc(F)ccc4F)CC3)nc2)c2c(C#N)cnn2c1] | N-[1-(5-{3-cyano-6- ethoxypyrazolo[1,5- a]pyridin-4-yl}pyridin-2-yl)- 4-[(5-{4-[2-(2,6- dioxopiperidin-3-yl)-3-oxo- 2,3-dihydro-1H-isoindol-5- yl]piperazin-1-yl}-N- (propan-2- yl)pentanamido)methyl] piperidin-4-yl]-2,5- difluorobenzamide | 984 [M + H]⁺, RT = 1.99 min (A) |
| Ex- ample 169 | 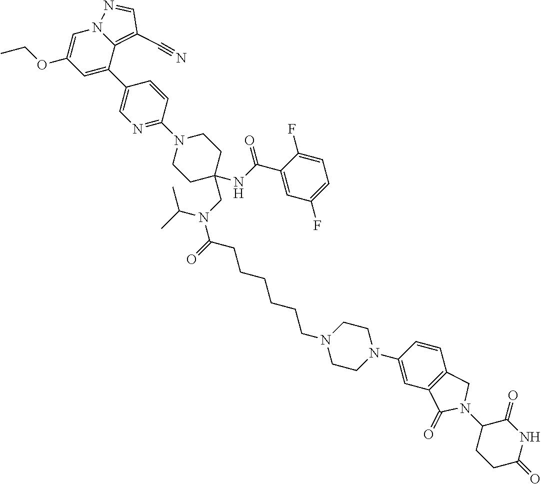 [SMILES: CCOc1cc(-c2ccc(N3CCC(CN(C(=O)CCCCCCN4CCN(c5ccc6c(c5)C(=O)N(C5CCC(=O)NC5=O)C6)CC4)C(C)C)(NC(=O)c4cc(F)ccc4F)CC3)nc2)c2c(C#N)cnn2c1] | N-[1-(5-{3-cyano-6- ethoxypyrazolo[1,5- a]pyridin-4-yl}pyridin-2-yl)- 4-[(7-{4-[2-(2,6- dioxopiperidin-3-yl)-3-oxo- 2,3-dihydro-1H-isoindol-5- yl]piperazin-1-yl}-N- (propan-2- yl)heptanamido)methyl] piperidin-4-yl]-2,5- difluorobenzamide | 1012 [M + H]⁺, RT = 2.11 min (A) |
| Ex- ample 170 | 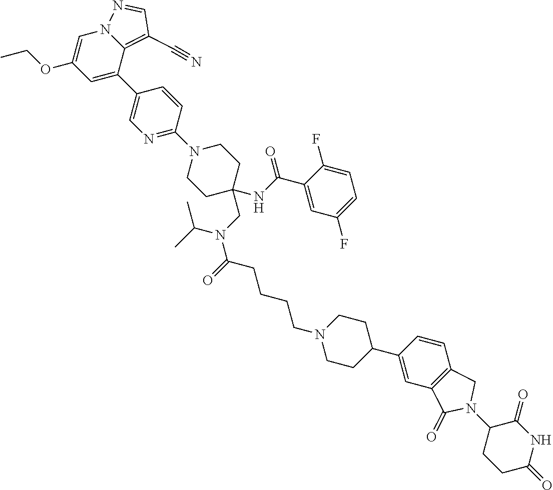 [SMILES: CCOc1cc(-c2ccc(N3CCC(CN(C(=O)CCCCN4CCC(c5ccc6c(c5)C(=O)N(C5CCC(=O)NC5=O)C6)CC4)C(C)C)(NC(=O)c4cc(F)ccc4F)CC3)nc2)c2c(C#N)cnn2c1] | N-[1-(5-{3-cyano-6- ethoxypyrazolo[1,5- a]pyridin-4-yl}pyridin-2-yl)- 4-[(5-{4-[2-(2,6- dioxopiperidin-3-yl)-3-oxo- 2,3-dihydro-1H-isoindol-5- yl]piperidin-1-yl}-N- (propan-2- yl)pentanamido)methyl] piperidin-4-yl]-2,5- difluorobenzamide | 983 [M + H]⁺, RT = 1.95 min (A) |
| Ex- ample 171 | 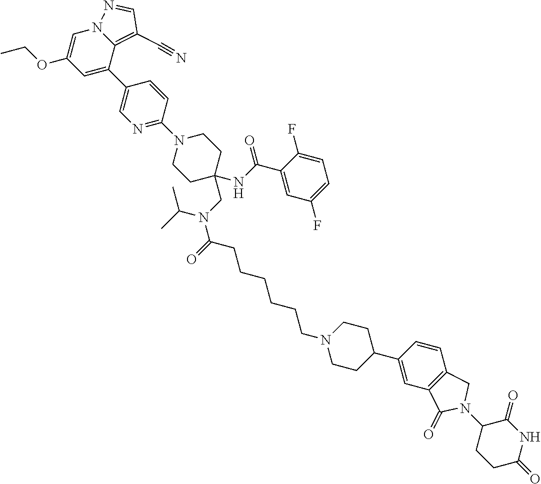 [SMILES: CCOc1cc(-c2ccc(N3CCC(CN(C(=O)CCCCCCN4CCC(c5ccc6c(c5)C(=O)N(C5CCC(=O)NC5=O)C6)CC4)C(C)C)(NC(=O)c4cc(F)ccc4F)CC3)nc2)c2c(C#N)cnn2c1] | N-[1-(5-{3-cyano-6- ethoxypyrazolo[1,5- a]pyridin-4-yl}pyridin-2-yl)- 4-[(7-{4-[2-(2,6- dioxopiperidin-3-yl)-3-oxo- 2,3-dihydro-1H-isoindol-5- yl]piperidin-1-yl}-N- (propan-2- yl)heptanamido)methyl] piperidin-4-yl]-2,5- difluorobenzamide | 1011 [M + H]⁺, RT = 2.04 min (A) |
| Ex- ample 172 | 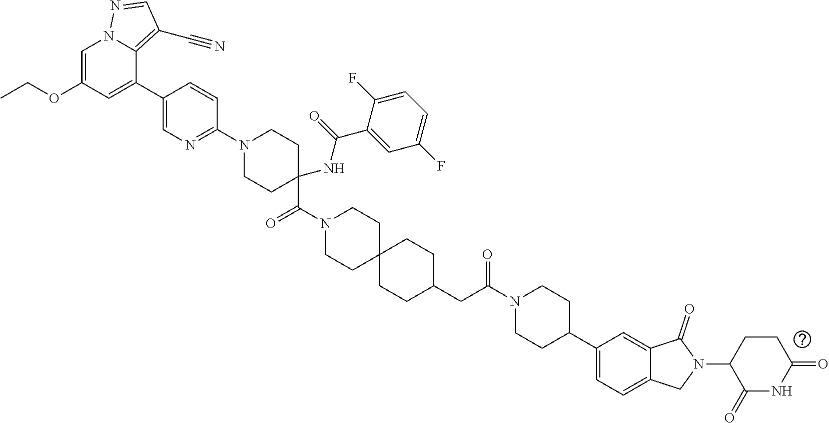 [SMILES: CCOc1cc(-c2ccc(N3CCC(NC(=O)c4cc(F)ccc4F)(C(=O)N4CCC5(CCC(CC(=O)N6CCC(c7ccc8c(c7)C(=O)N(C7CCC(=O)NC7=O)C8)CC6)CC5)CC4)CC3)nc2)c2c(C#N)cnn2c1] | N-[1-(5-{3-cyano-6- ethoxypyrazolo[1,5- a]pyridin-4-yl}pyridin-2-yl)- 4-[9-(2-{4-[2-(2,6- dioxopiperidin-3-yl)-3-oxo- 2,3-dihydro-1H-isoindol-5- yl]piperidin-1-yl}-2- oxoethyl)-3- azaspiro[5.5]undecane-3- carbonyl]piperidin-4-yl]-2,5- difluorobenzamide | 1049.2 [M + H]⁺, RT = 2.02 min (A) |
| Ex- ample 173 | 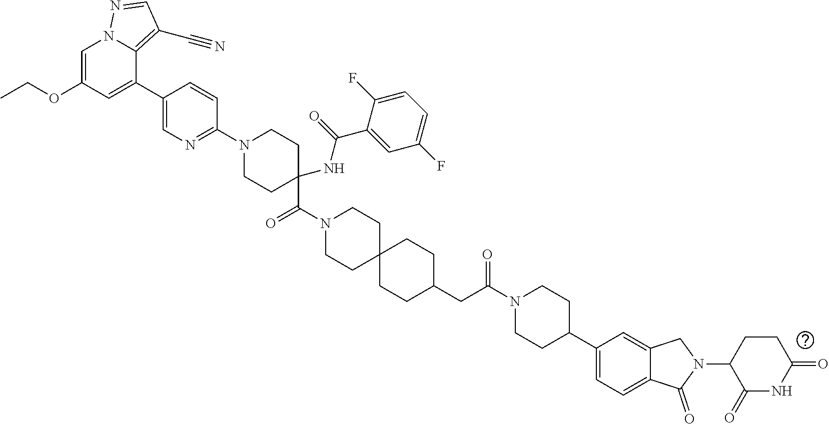 [SMILES: CCOc1cc(-c2ccc(N3CCC(NC(=O)c4cc(F)ccc4F)(C(=O)N4CCC5(CCC(CC(=O)N6CCC(c7ccc8c(c7)CN(C7CCC(=O)NC7=O)C8=O)CC6)CC5)CC4)CC3)nc2)c2c(C#N)cnn2c1] | N-[1-(5-{3-cyano-6- ethoxypyrazolo[1,5- a]pyridin-4-yl}pyridin-2-yl)- 4-[9-(2-{4-[2-(2,6- dioxopiperidin-3-yl)-1-oxo- 2,3-dihydro-1H-isoindol-5- yl]piperidin-1-yl}-2- oxoethyl)-3- azaspiro[5.5]undecane-3- carbonyl]piperidin-4-yl]-2,5- difluorobenzamide | 1049.2 [M + H]⁺, RT = 1.99 min (A) |
| Ex- ample 174 | 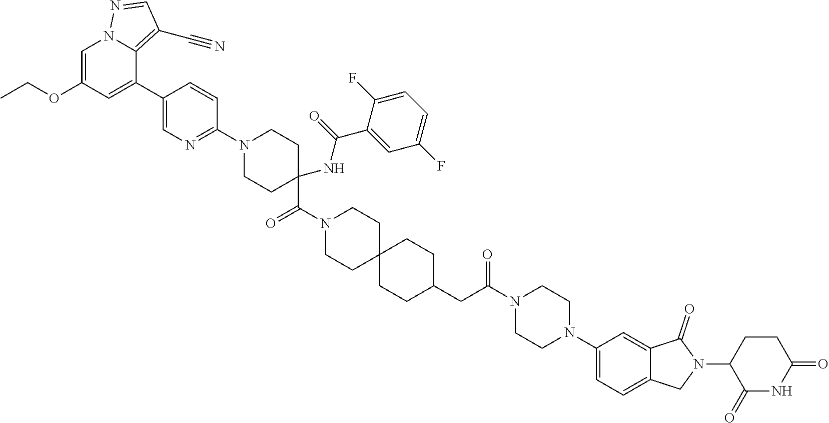 [SMILES: CCOc1cc(-c2ccc(N3CCC(NC(=O)c4cc(F)ccc4F)(C(=O)N4CCC5(CCC(CC(=O)N6CCN(c7ccc8c(c7)C(=O)N(C7CCC(=O)NC7=O)C8)CC6)CC5)CC4)CC3)nc2)c2c(C#N)cnn2c1] | N-[1-(5-{3-cyano-6- ethoxypyrazolo[1,5- a]pyridin-4-yl}pyridin-2-yl)- 4-[9-(2-{4-[2-(2,6- dioxopiperidin-3-yl)-3-oxo- 2,3-dihydro-1H-isoindol-5- yl]piperazin-1-yl}-2- oxoethyl)-3- azaspiro[5.5]undecane-3- carbonyl]piperidin-4-yl]-2,5- difluorobenzamide | 1050.7 [M + H]⁺, RT = 1.97 min (A) |
| Ex- ample 175 | 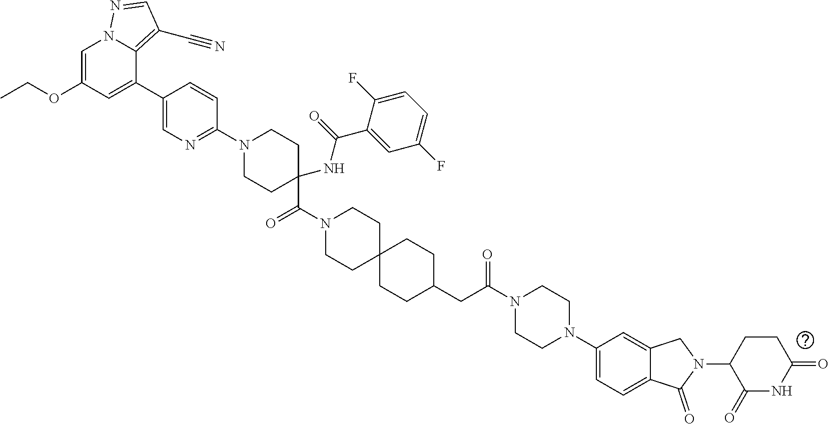 [SMILES: CCOc1cc(-c2ccc(N3CCC(NC(=O)c4cc(F)ccc4F)(C(=O)N4CCC5(CCC(CC(=O)N6CCN(c7ccc8c(c7)CN(C7CCC(=O)NC7=O)C8=O)CC6)CC5)CC4)CC3)nc2)c2c(C#N)cnn2c1] | N-[1-(5-{3-cyano-6- ethoxypyrazolo[1,5- a]pyridin-4-yl}pyridin-2-yl)- 4-[9-(2-{4-[2-(2,6- dioxopiperidin-3-yl)-1-oxo- 2,3-dihydro-1H-isoindol-5- yl]piperazin-1-yl}-2- oxoethyl)-3- azaspiro[5.5]undecane-3- carbonyl]piperidin-4-yl]-2,5- difluorobenzamide | 1050.3 [M + H]⁺, RT = 2.01 min (A) |
| Ex- ample 176 | 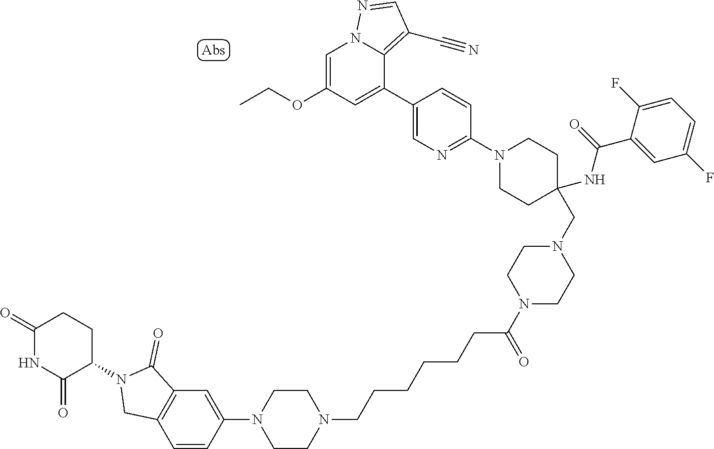 [SMILES: CCOc1cc(-c2ccc(N3CCC(CN4CCN(C(=O)CCCCCCN5CCN(c6ccc7c(c6)C(=O)N([C@H]6CCC(=O)NC6=O)C7)CC5)CC4)(NC(=O)c4cc(F)ccc4F)CC3)nc2)c2c(C#N)cnn2c1] | (S)-N-(1-(5-(3-cyano-6- ethoxypyrazolo[1,5- a]pyridin-4-yl)pyridin-2-yl)- 4-((4-(7-(4-(2-(2,6- dioxopiperidin-3-yl)-3- oxoisoindolin-5-yl)piperazin- 1-yl)heptanoyl)piperazin-1- yl)methyl)piperidin-4-yl)-2,5- difluorobenzamide | 1039 [M + H]⁺, RT = 0.73 min (Method Column 6) |
| Ex- ample 177 | 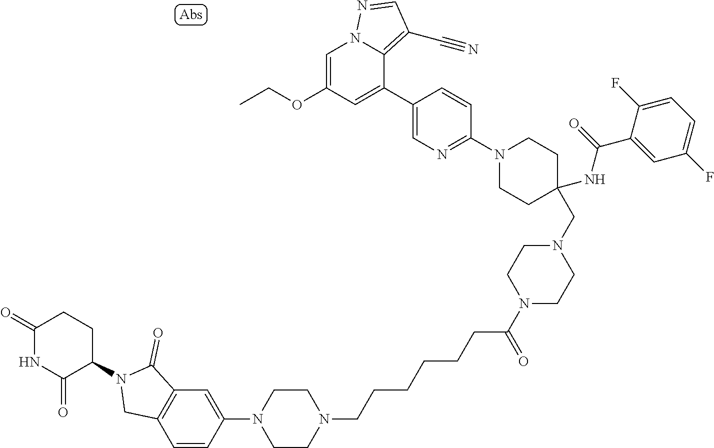 [SMILES: CCOc1cc(-c2ccc(N3CCC(CN4CCN(C(=O)CCCCCCN5CCN(c6ccc7c(c6)C(=O)N([C@@H]6CCC(=O)NC6=O)C7)CC5)CC4)(NC(=O)c4cc(F)ccc4F)CC3)nc2)c2c(C#N)cnn2c1] | (R)-N-(1-(5-(3-cyano-6- ethoxypyrazolo[1,5- a]pyridin-4-yl)pyridin-2-yl)- 4-((4-(7-(4-(2-(2,6- dioxopiperidin-3-yl)-3- oxoisoindolin-5-yl)piperazin- 1-yl)heptanoyl)piperazin-1- yl)methyl)piperidin-4-yl)-2,5- difluorobenzamide | 1039 [M + H]⁺, RT = 0.77 min (Method Column 6) |
| Ex- ample 178 | 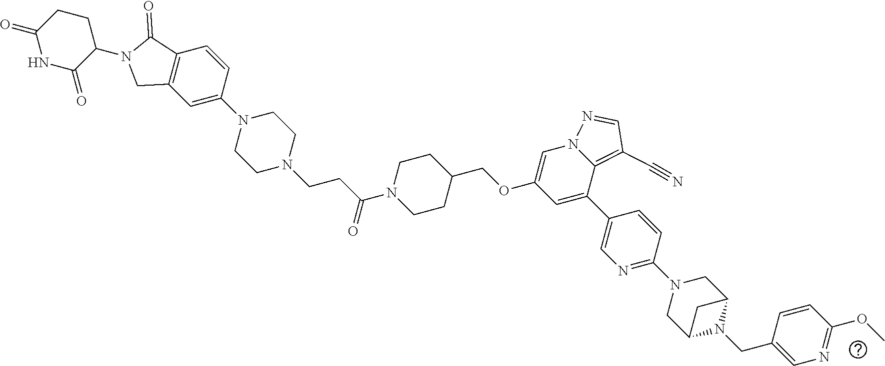 [SMILES: COc1ccc(CN2[C@@H]3C[C@H]2CN(c2ccc(-c4cc(OCC5CCN(C(=O)CCN6CCN(c7ccc8c(c7)CN(C7CCC(=O)NC7=O)C8=O)CC6)CC5)cn5ncc(C#N)c45)cn2)C3)cn1] | 6-{[1-(3-{4-[2-(2,6- dioxopiperidin-3-yl)- 1-oxo-2,3- dihydro-1H-isoindol-5- yl]piperazin-1- yl}propanoyl)piperidin-4- yl]methoxy}-4-{6- [(1R,5S)-6- [(6-methoxypyridin-3- yl)methyl]-3,6- diazabicyclo[3.1.1] heptan-3- yl]pyridin-3- yl}pyrazolo[1,5- a]pyridine-3- carbonitrile | 933.3 [M + H]⁺, RT = 1.63 min (Method A) |
| Ex- ample 179 | 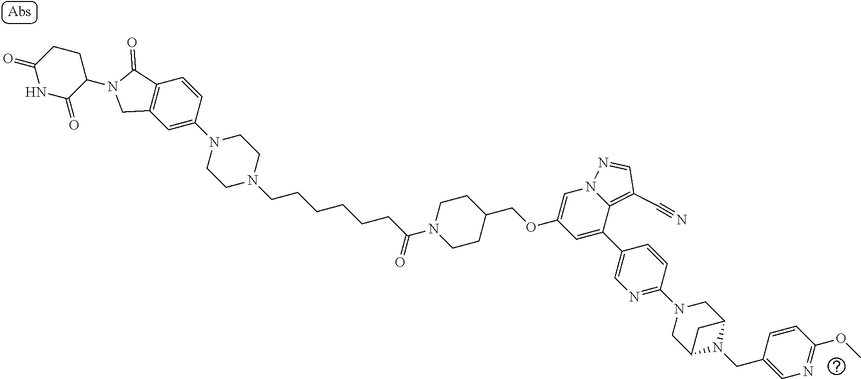 [SMILES: COc1ccc(CN2[C@@H]3C[C@H]2CN(c2ccc(-c4cc(OCC5CCN(C(=O)CCCCCCN6CCN(c7ccc8c(c7)CN(C7CCC(=O)NC7=O)C8=O)CC6)CC5)cn5ncc(C#N)c45)cn2)C3)cn1] | 6-{[1-(7-{4-[2-(2,6- dioxopiperidin-3-yl)- 1-oxo-2,3- dihydro-1H-isoindol-5- yl]piperazin-1- yl}heptanoyl)piperidin-4- yl]methoxy}-4-{6- [(1R,5S)-6- [(6-methoxypyridin-3- yl)methyl]-3,6- diazabicyclo[3.1.1] heptan-3- yl]pyridin-3- yl}pyrazolo[1,5- a]pyridine-3- carbonitrile | 989.4 [M + H]⁺, RT = 1.73 min (Method A) |
| Examle 180 | 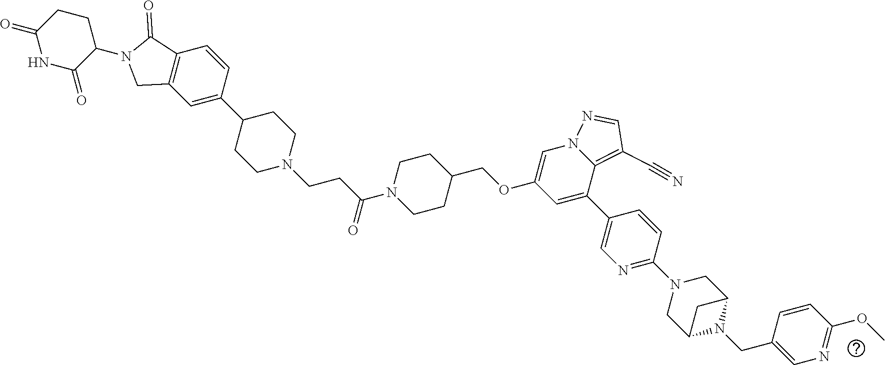 [SMILES: COc1ccc(CN2[C@@H]3C[C@H]2CN(c2ccc(-c4cc(OCC5CCN(C(=O)CCN6CCC(c7ccc8c(c7)CN(C7CCC(=O)NC7=O)C8=O)CC6)CC5)cn5ncc(C#N)c45)cn2)C3)cn1] | 6-{[1-(3-{4-[2-(2,6- dioxopiperidin-3-yl)- 1-oxo-2,3- dihydro-1H-isoindol-5- yl]piperidin-1- yl}propanoyl)piperidin-4- yl]methoxy}-4-{6- [(1R,5S)-6- [(6-methoxypyridin-3- yl)methyl]-3,6- diazabicyclo[3.1.1] heptan-3- yl]pyridin-3- yl}pyrazolo[1,5- a]pyridine-3- carbonitrile | 932.2 [M + H]⁺, RT = 1.59 min (Method B) |
| Ex- ample 181 | 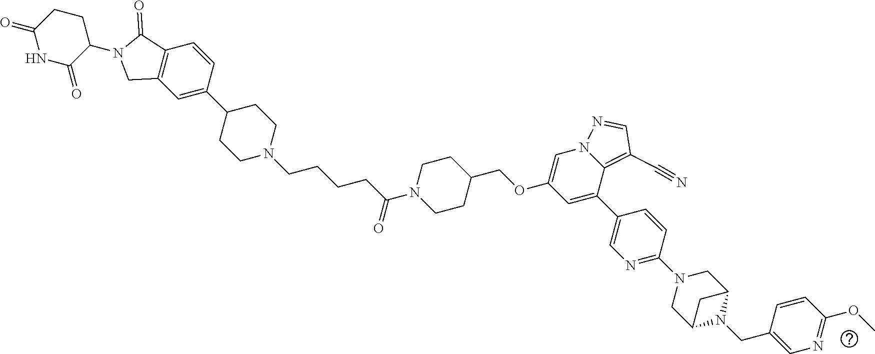 [SMILES: COc1ccc(CN2[C@@H]3C[C@H]2CN(c2ccc(-c4cc(OCC5CCN(C(=O)CCCCN6CCC(c7ccc8c(c7)CN(C7CCC(=O)NC7=O)C8=O)CC6)CC5)cn5ncc(C#N)c45)cn2)C3)cn1] | 6-{[1-(5-{4-[2-(2,6- dioxopiperidin-3-yl)- 1-oxo-2,3- dihydro-1H-isoindol-5- yl]piperidin-1- yl}pentanoyl)piperidin-4- yl]methoxy}-4-{6- [(1R,5S)-6- [(6-methoxypyridin-3- yl)methyl]-3,6- diazabicyclo[3.1.1] heptan-3- yl]pyridin-3- yl}pyrazolo[1,5- a]pyridine-3- carbonitrile | 960.4 [M + H]⁺, RT = 1.53 min (Method A) |
| Ex- ample 182 | 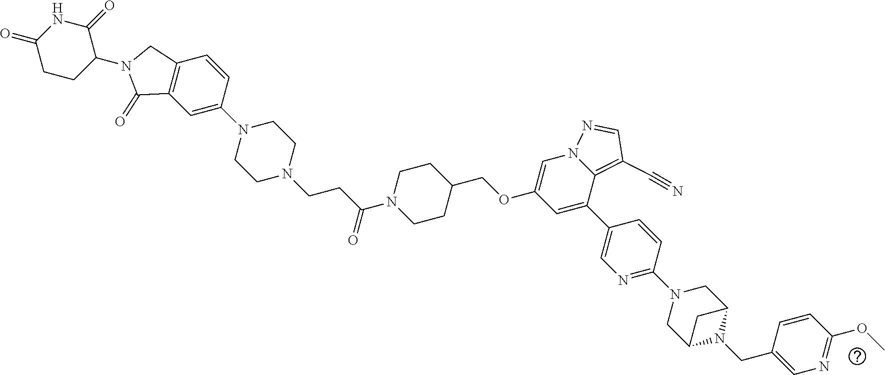 [SMILES: COc1ccc(CN2[C@@H]3C[C@H]2CN(c2ccc(-c4cc(OCC5CCN(C(=O)CCN6CCN(c7ccc8c(c7)C(=O)N(C7CCC(=O)NC7=O)C8)CC6)CC5)cn5ncc(C#N)c45)cn2)C3)cn1] | 6-{[1-(3-{4-[2-(2,6- dioxopiperidin-3-yl)- 3-oxo-2,3- dihydro-1H-isoindol-5- yl]piperazin-1- yl}propanoyl)piperidin-4- yl]methoxy}-4-{6- [(1R,5S)-6- [(6-methoxypyridin-3- yl)methyl]-3,6- diazabicyclo[3.1.1] heptan-3- yl]pyridin-3- yl}pyrazolo[1,5- a]pyridine-3- carbonitrile | 933.3 [M + H]⁺, RT = 1.65 min (Method A) |
| Ex- ample 183 | 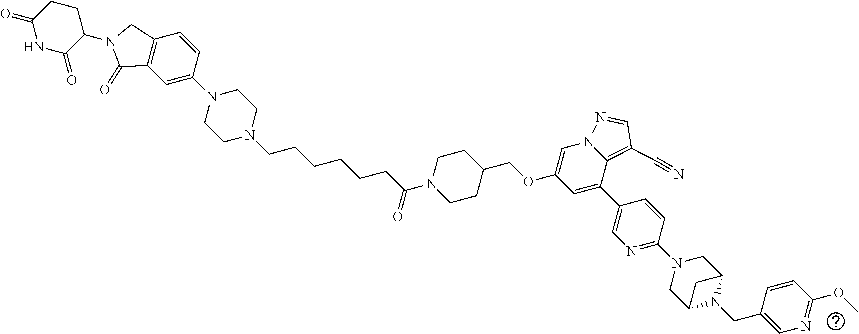 [SMILES: COc1ccc(CN2[C@@H]3C[C@H]2CN(c2ccc(-c4cc(OCC5CCN(C(=O)CCCCCCN6CCN(c7ccc8c(c7)C(=O)N(C7CCC(=O)NC7=O)C8)CC6)CC5)cn5ncc(C#N)c45)cn2)C3)cn1] | 6-{[1-(7-{4-[2-(2,6- dioxopiperidin-3-yl)- 3-oxo-2,3- dihydro-1H-isoindol-5- yl]piperazin-1- yl}heptanoyl)piperidin-4- yl]methoxy}-4-{6- [(1R,5S)-6- [(6-methoxypyridin-3- yl)methyl]-3,6- diazabicyclo[3.1.1] heptan-3- yl]pyridin-3- yl}pyrazolo[1,5- a]pyridine-3- carbonitrile | 989.4 [M + H]⁺, RT = 1.77 min (Method A) |
| Ex- ample 184 | 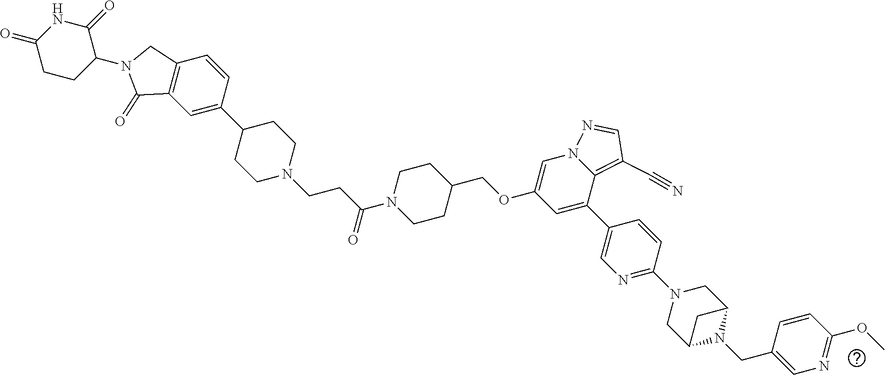 [SMILES: COc1ccc(CN2[C@@H]3C[C@H]2CN(c2ccc(-c4cc(OCC5CCN(C(=O)CCN6CCC(c7ccc8c(c7)C(=O)N(C7CCC(=O)NC7=O)C8)CC6)CC5)cn5ncc(C#N)c45)cn2)C3)cn1] | 6-{[1-(3-{4-[2-(2,6- dioxopiperidin-3-yl)- 3-oxo-2,3- dihydro-1H-isoindol-5- yl]piperidin-1- yl}propanoyl)piperidin-4- yl]methoxy}-4-{6- [(1R,5S)-6- [(6-methoxypyridin-3- yl)methyl]-3,6- diazabicyclo[3.1.1] heptan-3- yl]pyridin-3- yl}pyrazolo[1,5- a]pyridine-3- carbonitrile | 932.4 [M + H]⁺, RT = 1.59 min (Method A) |
| Ex- ample 185 | 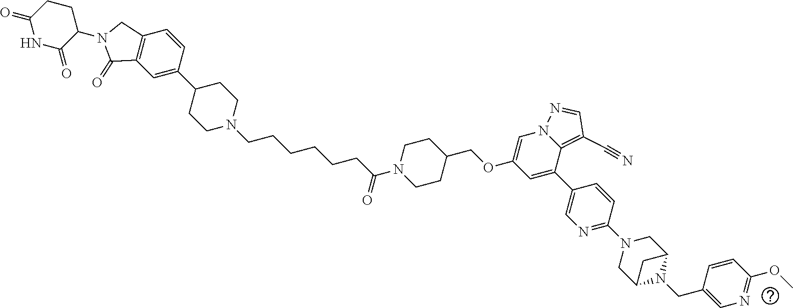 [SMILES: COc1ccc(CN2[C@@H]3C[C@H]2CN(c2ccc(-c4cc(OCC5CCN(C(=O)CCCCCCN6CCC(c7ccc8c(c7)C(=O)N(C7CCC(=O)NC7=O)C8)CC6)CC5)cn5ncc(C#N)c45)cn2)C3)cn1] | 6-{[1-(7-{4-[2-(2,6- dioxopiperidin-3-yl)- 3-oxo-2,3- dihydro-1H-isoindol-5- yl]piperidin-1- yl}heptanoyl)piperidin-4- yl]methoxy}-4-{6- [(1R,5S)-6- [(6-methoxypyridin-3- yl)methyl]-3,6- diazabicyclo[3.1.1] heptan-3- yl]pyridin-3- yl}pyrazolo[1,5- a]pyridine-3- carbonitrile | 988.3 [M + H]⁺, RT = 1.3 min (Method B) |

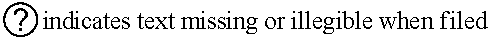

### Cellular RET Degradation Assay

Cellular RET degradation compound potency was determined using the bead-based luminescent amplification assay-AlphaLISA Surefire Ultra Detection Kit (Perkin-Elmer). The CCDC6-RET fusion expressing papillary thyroid carcinoma (TPC-1) cell line was selected for screening of potential degraders: Parental (TPC-1/CCDC6-RET). Cells were plated in 384 well culture plates (Greiner Bio-One) at 10,000 cells/well in 50 ul/well DMEM (Fisher Scientific) with 2% FBS serum (Gibco). After cellular adhesion (4 hrs), cells were treated with compound by dispensing 25 nl of serially diluted compound (1:3 dilution: 11point) directly into the cell culture wells using an Echo-655 acoustic liquid dispenser (Beckman). Low normalization wells were media only and high normalization wells were cells treated with DMSO only. Treated cell plates were placed in an incubator at 37 °C and 5% CO₂ for 24 hrs. At 24 hrs, cell media was removed by a FELIX automated liquid hander (Analytik Jena) and 25 ul of cell lysis buffer (provided in AlphaLisa kit) was added to each well. Sul cell lysate were transferred into a 384-well microplate (Perkin Elmer) before addition of 2.5 ul AlphaLISA Acceptor Bead mix. The plates were sealed and kept at room temperature for 1 hr before addition of donor bead mixture. Donor bead addition was performed in the dark and incubated for >1 hr before AlphaLISA luminescence was measured on an Envision plate reader with an Alphascreen aperture at RT (excitation 680 nm, emission 615 nm). RET protein abundance (and therefore degradation) directly corresponds to luminescence intensity.

Intensities were normalized relative to the average values from 16 wells containing media alone (blank-low) and 16 wells containing cells +DMSO (high). % Degradation for each well was determined using the standard equation, where

$$\% Degradation = \frac{total - well count}{total - blank} \times 100.$$

DC₅₀ (IC50) values for each compound tested are calculated by analyzing plots of % degradation versus compound concentration, and fitting to the standard 4-parameter logistic curve where,

$$\% Degradation = Bottom + \frac{{[ Compound ]}^{N} \times ( Top - Bottom )}{{Halfmax}^{N} + {[ Compound ]}^{N}} .$$

Top and bottom are the % degradation at infinitely large and infinitely small compound concentrations, respectively, while Halfmax is the compound concentration yielding % degradation corresponding to ½ Top-Bottom, and N is the Hill coefficient. DC₅₀ values are calculated according to the relationship,

$${DC}_{50} = Halfmax ( \sqrt[N]{\frac{50 - Bottom}{Top - 50}} ) .$$

Compound replicates were performed in independent assays. Maximum degradation (YMax) was defined as the maximal % degradation value from the 4-parameter logistic equation, as previously described.

### Activity Table.

Each of the compounds in Table 2, was tested in one or more of the degradation assays shown above, for example, the tRET AlphaLISA Degradation Assay with the parental RET TPC-1 cell line, and was found to have activity therein. The half-maximal degradation concentration (DC₅₀) and % degradation response (Ymax) are included in Table 2. All of the compounds in Table 2 were shown to have a DC₅₀<1 μM and Ymax>60%, with some compounds having a DC₅₀ value D: DC₅₀≤2 nM, some a DC₅₀ value C: 2 nM<DC₅₀≤10 nM, and some a DC₅₀ value B: 10 nM<DC₅₀≤100 nM and some a DC₅₀ value A: 100 nM<DC₅₀≤2000 nM. Additionally the compounds were shown to have a RET degradation efficiency Y value>60%, with some compounds having 60%<Y≤90% (shown as *), some compounds having Y>90% (shown as **).

TABLE 2

| | Cell Density TPC- | |
|---|---|---|
| | 1/CCDC6-RET DC₅₀ | Cell Density TPC- |
| Example # | (nM) | 1/CCDC6-RET Ymax % |
| 2 | C | * |
| 3 | C | ** |
| 4 | C | ** |
| 5 | C | ** |
| 6 | C | ** |
| 7 | C | ** |
| 8 | C | ** |
| 9 | C | ** |
| 10 | D | * |
| 11 | D | * |
| 12 | D | ** |
| 13 | D | ** |
| 14 | C | * |
| 15 | C | ** |
| 16 | C | ** |
| 17 | C | ** |
| 18 | D | ** |
| 19 | C | ** |
| 20 | C | ** |
| 21 | B | * |
| 22 | B | * |
| 23 | B | ** |
| 24 | B | * |
| 25 | C | ** |
| 26 | C | ** |
| 27 | C | * |
| 28 | B | * |
| 29 | B | ** |
| 30 | A | * |
| 31 | C | * |
| 32 | B | * |
| 33 | C | * |
| 34 | B | ** |
| 35 | B | ** |
| 36 | C | * |
| 37 | B | * |
| 38 | D | ** |
| 39 | D | * |
| 40 | C | ** |
| 41 | D | ** |
| 42 | D | |
| 43 | D | ** |
| 44 | D | * |
| 45 | D | |
| 47 | C | ** |
| 48 | C | ** |
| 49 | C | ** |
| 50 | C | * |
| 51 | B | * |
| 52 | C | ** |
| 53 | B | * |
| 54 | A | * |
| 55 | C | * |
| 56 | C | * |
| 57 | A | * |
| 58 | D | ** |
| 59 | D | ** |
| 60 | D | ** |
| 61 | B | * |
| 62 | C | * |
| 63 | C | * |
| 64 | D | ** |
| 65 | C | * |
| 66 | D | * |
| 67 | C | * |
| 69 | D | ** |
| 70 | D | ** |
| 71 | D | ** |
| 72 | B | ** |
| 74 | D | ** |
| 76 | B | * |
| 77 | D | * |
| 78 | D | ** |
| 81 | D | ** |
| 82 | D | ** |
| 83 | D | ** |
| 84 | D | ** |
| 85 | D | * |
| 86 | D | ** |
| 87 | C | ** |
| 88 | B | ** |
| 89 | B | ** |
| 90 | B | ** |
| 91 | D | * |
| 92 | C | ** |
| 93 | C | ** |
| 94 | C | * |
| 95 | C | * |
| 96 | B | * |
| 97 | C | * |
| 98 | C | ** |
| 99 | D | * |
| 100 | C | ** |
| 101 | B | ** |
| 103 | B | ** |
| 104 | C | * |
| 105 | C | * |
| 108 | B | * |
| 109 | B | * |
| 110 | C | * |
| 111 | C | * |
| 112 | C | ** |
| 113 | D | ** |
| 114 | D | * |
| 115 | B | * |
| 116 | C | ** |
| 117 | C | ** |
| 118 | C | * |
| 119 | C | ** |
| 120 | D | ** |
| 121 | B | ** |
| 122 | C | ** |
| 123 | C | ** |
| 124 | D | ** |
| 125 | B | ** |
| 126 | A | * |
| 128 | A | * |
| 129 | A | * |
| 137 | C | ** |
| 138 | NA | NA |
| 139 | B | ** |
| 140 | C | ** |
| 142 | A | NA |
| 143 | C | ** |
| 145 | C | * |
| 146 | B | ** |
| 147 | B | * |
| 148 | C | * |
| 149 | D | ** |
| 150 | B | ** |
| 152 | C | * |
| 153 | C | ** |
| 154 | B | * |
| 155 | D | * |
| 156 | C | * |
| 157 | C | ** |
| 158 | D | * |
| 159 | C | * |
| 160 | C | ** |
| 161 | D | * |
| 162 | B | * |
| 163 | C | * |
| 164 | C | ** |
| 165 | D | ** |
| 166 | C | * |
| 167 | C | * |
| 168 | C | ** |
| 169 | B | * |
| 170 | A | * |
| 171 | C | ** |

### EMBODIMENTS

### Embodiment 1

A compound of Formula I:

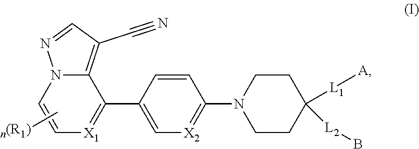

or a pharmaceutically acceptable salt, solvate, isomer, enantiomer, or tautomer thereof, wherein
R₁ is —OCH₃, —OCH₂CH₃, —OCF₃, —OCH₂CH₂OH, —OCH₂CH₂F, halogen, or 1-methyl-pyrazol-4-yl;
A is independently H, OH, C₁-C₆ alkyl, C₃-C₈ cycloalkyl, aryl, or heteroaryl, wherein the alkyl, cycloalkyl, aryl or heteroaryl is optionally substituted with one or more R₂; wherein R₂ is independently halogen, OH, C₁-C₃ alkyl, or C₁-C₃ alkoxy;
B is a moiety that is capable of binding a E3 ligase;
L₁ is independently a bond, —C₁-C₃ alkylenyl-, —NHCO—, —CONH—, —O—, or —NH—;
L₂ is independently —(CH₂)ₚ-(4- to 12-heterocycloalkyl)-Y₁—C(O)—Y₂—, —(CH₂)ₚ-(4- to 12-heterocycloalkyl)-C(O)—Y₁-Y₂—, —(CH₂)ₚ-(4- to 12-heterocycloalkyl)-C(O)—Y₁—C(O)—Y₂—, —(CH₂)ₚ-(4- to 12-heterocycloalkyl)-Y₁-Y₂—, —(OCH₂CH₂)ₚ-(4- to 12-heterocycloalkyl)-Y₂—, —(CH₂OCH₂C(O))ₚ-(4- to 12-heterocycloalkyl)-Y₂—, —(CH₂OCH₂C(O)NH)ₚ—Y₂—, —(CH₂)ₚ-(4- to 12-heterocycloalkyl)-Y₁—(CH₂CH₂O)ₚ—C(O)—Y₂—, —(CH₂)ₚ-(4- to 12-heterocycloalkyl)-Y₁—(CH₂CH₂O)ₚ—Y₂—, —(CH₂)ₚ-(4- to 12-heterocycloalkyl)-Y₁—(OCH₂CH₂)ₚ—Y₂—, —(CH₂)ₚ-(4- to 12-heterocycloalkyl)-Y₁—(OCH₂CH₂)ₚ—C(O)—(OCH₂CH₂)ₚ—Y₂, —(CH₂OCH₂)ₚ-(heteroaryl)-Y₂—, —NHC(O)-(4- to 12-heterocycloalkyl)-Y₁—C(O)—Y₂—, —NHC(O)—(CH₂)ₚ-(4- to 12-heterocycloalkyl)-Y₁—C(O)—Y₂—, —NHC(O)—(CH₂)ₚ-(4- to 12-heterocycloalkyl)-Y₂—, —C(O)NH—Y₁-Y₂—;
   —(CH₂)ₚ—NH—Y₁-Y₂—;
   —C(O)NH—(CH₂CH₂O)ₚ—Y₂—, —C(O)—(CH₂CH₂O)ₚ—Y₂—, —(CH₂OCH₂)ₚ—NR₃C(O)—(CH₂CH₂O)ₚ—Y₂—, —(CH₂)ₚ—NR₃C(O)—(CH₂CH₂O)ₚ—Y₂—, —(CH₂OCH₂CH₂)ₚ-(4- to 12-heterocycloalkyl)-Y₁—C(O)—(CH₂CH₂O)ₚ—Y₂—, or
   —(CH₂OCH₂)ₚ—C(O)NH—(CH₂CH₂O)ₚ—Y₂—,
   wherein
      R₃ is —H or —C₁-C₃ alkyl,
      Y₁ is independently a bond, —C₁₋₈ alkylenyl, 4- to 12-membered heterocycloalkyl, or —C₃-C₁₀ cycloalkyl;
      Y₂ is independently
         a bond,
         —C₁-C₃ alkyl-,
         —(CH₂)ₘ—O—,
         —O—(CH₂)ₘ-(4- to 6-membered heterocycloalkyl),
         —(CH₂)ₘ—O—(CH₂)ₘ—NH—,
         —(CH₂)ₘ—NH—,
         —(CH₂)ₘ—NHC(O)—(CH₂)ₘO—,
         —(CH₂)ₘ—C(O)NH—(CH₂)ₘ—,
         —(CH₂)ₘ—C₃-C₁₀ cycloalkyl-,
         —(CH₂)ₘ-aryl,
         —(CH₂)ₘ-heteroaryl-,
         —(CH₂)ₘ-(4- to 6-membered heterocycloalkyl)-,
         —(CH₂)ₘ-(4- to 6-membered heterocycloalkyl)-C(O)—,
         —(CH₂)ₘ-(4- to 6-membered heterocycloalkyl)-C(O)—(CH₂)ₘ—,
         —(CH₂)ₘ-(4- to 6-membered heterocycloalkyl)-C(O)—(CH₂)ₘ—O—,
         —(CH₂)ₘ—C₃-C₁₀ cycloalkyl-C(O)NH—, or
         —(CH₂)ₘ-(4- to 6-membered heterocycloalkyl)-O—,
X₁ is CH or N;
X₂ is CH or N;
m is independently at each occurrence an integer from 0 to 16;
n is an integer from 0 to 2; and p is an integer from 0 to 3.

### Embodiment 2

The compound according to Embodiment 1, wherein the compound has Formula I(a):

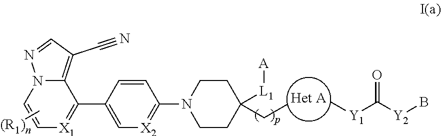

wherein Het A is 4- to 6-membered heterocycloalkyl.

### Embodiment 3

The compound according to any one of the preceding embodiments, wherein the compound has Formula I(b):

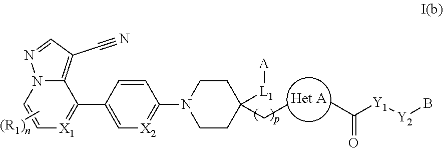

wherein Het A is 4- to 6-membered heterocycloalkyl.

### Embodiment 4

The compound according to any one of the preceding embodiments, wherein the compound has Formula I(c):

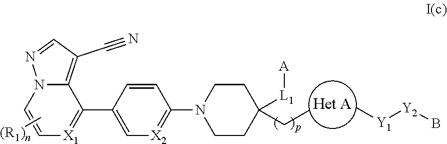

wherein Het A is 4- to 6-membered heterocycloalkyl.

### Embodiment 5

The compound according to any one of the preceding embodiments, wherein the compound has Formula I(d):

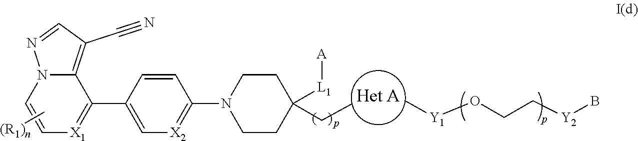

wherein Het A is 4- to 6-membered heterocycloalkyl.

### Embodiment 6

The compound according to any one of the preceding embodiments, wherein the compound has Formula I(e):

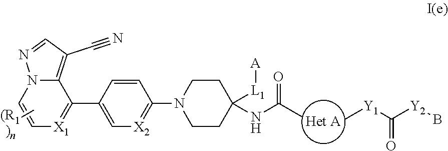

wherein Het A is 4- to 6-membered heterocycloalkyl.

### Embodiment 7

The compound according to any one of the preceding embodiments, wherein the compound has Formula I(f):

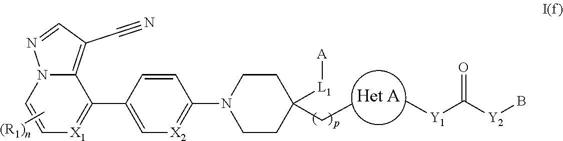

wherein Het A is 4- to 6-membered heterocycloalkyl.

### Embodiment 8

The compound according to any one of the preceding embodiments, wherein the compound has Formula I(g):

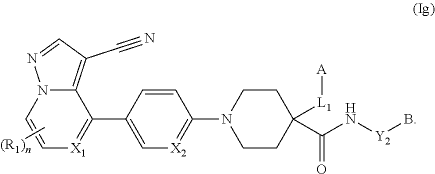

### Embodiment 9

The compound according to any one of the preceding embodiments, wherein the compound has Formula I(h):

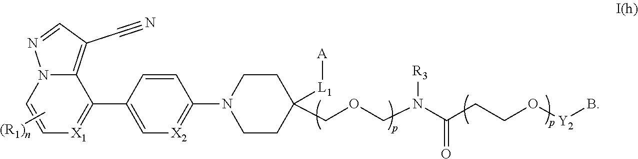

### Embodiment 10

The compound according to any one of the preceding embodiments, wherein L₁ is —CONH—.

### Embodiment 11

The compound according to any one of the preceding embodiments, wherein L₁ is —NHC(O)—.

### Embodiment 12

The compound according to any one of the preceding embodiments, wherein X₁ is CH.

### Embodiment 13

The compound according to any one of the preceding embodiments, wherein X₂ is N.

### Embodiment 14

The compound according to any one of the preceding embodiments, wherein R₁ is —OCH₂CH₃.

### Embodiment 15

The compound according to any one of the preceding embodiments, wherein moiety B capable of binding a E3 ligase is of the structure:

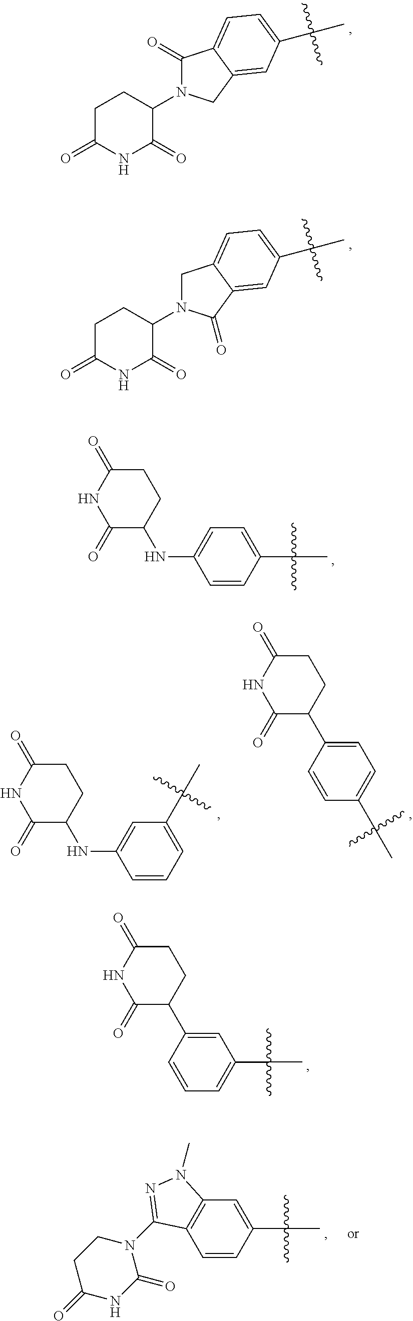

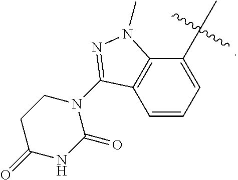 [SMILES: *c1cccc2c(N3CCC(=O)NC3=O)nn(C)c12]

### Embodiment 16

The compound according to any one of the preceding embodiments, wherein moiety B capable of binding a E3 ligase is of the structure:

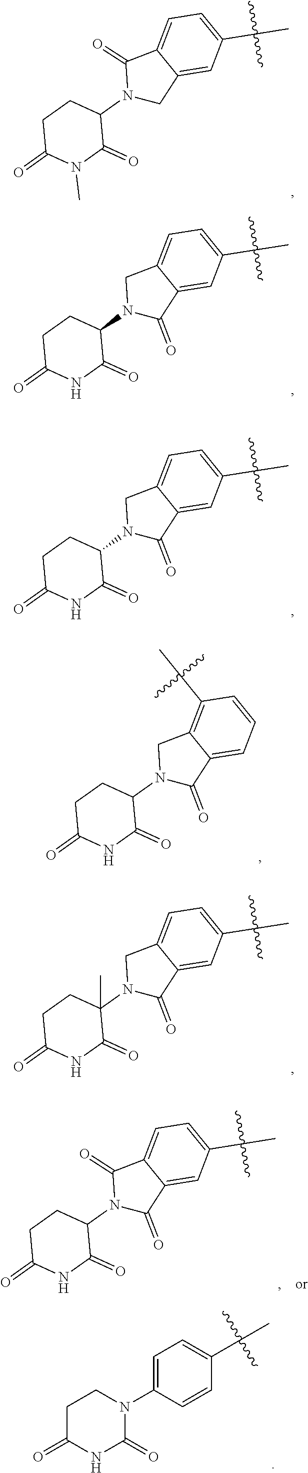

### Embodiment 17

The compound according to any one of the preceding embodiments, wherein A is of the structure:

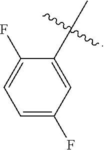 [SMILES: *c1cc(F)ccc1F]

### Embodiment 18

The compound according to any one of the preceding embodiments, wherein the compound is selected from:
N-(1-(5-(3-cyano-6-ethoxypyrazolo[1,5-a]pyridin-4-yl)pyridin-2-yl)-4-((4-(7-(4-(2-(2,6-dioxopiperidin-3-yl)-1-oxoisoindolin-5-yl)piperazin-1-yl)heptanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(7-(4-(2-(2,6-dioxopiperidin-3-yl)-1-oxoisoindolin-5-yl)piperazin-1-yl)heptanoyl)piperazin-1-yl)methyl)piperidin-4-yl)-3-fluoropicolinamide;
N-(1-(5-(3-cyano-6-ethoxypyrazolo[1,5-a]pyridin-4-yl)pyridin-2-yl)-4-((4-(7-(4-(2-(2,6-dioxopiperidin-3-yl)-1-oxoisoindolin-5-yl)piperazin-1-yl)heptanoyl)piperazin-1-yl)methyl)piperidin-4-yl)-3-methylbutanamide;
N-(1-(5-(3-cyano-6-ethoxypyrazolo[1,5-a]pyridin-4-yl)pyridin-2-yl)-4-((4-(3-(4-(2-(2,6 dioxopiperidin-3-yl)-3-oxoisoindolin-5-yl)piperidin-1-yl)propanoyl)piperazin-1-yl)methyl)piperidin-4-yl)-3-fluoropicolinamide;
N-(1-(5-(3-cyano-6-ethoxypyrazolo[1,5-a]pyridin-4-yl)pyridin-2-yl)-4-((4-(5-(4-(2-(2,6-dioxopiperidin-3-yl)-1-oxoisoindolin-5-yl)piperazin-1-yl)pentanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(2-(4-(2-(2,6-dioxopiperidin-3-yl)-1-oxoisoindolin-5-yl)piperazin-1-yl)acetyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(7-(4-(2-(2,6-dioxopiperidin-3-yl)-1-oxoisoindolin-5-yl)piperazin-1-yl)heptanoyl)piperazin-1-yl)methyl)piperidin-4-yl)cyclopropanecarboxamide;
N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)propanoyl)piperazin-1-yl)methyl)piperidin-4-yl)-3-methylbutanamide;
N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)propanoyl)piperazin-1-yl)methyl)piperidin-4-yl)cyclopropanecarboxamide;
N-(1-(5-(3-cyano-6-ethoxypyrazolo[1,5-a]pyridin-4-yl)pyridin-2-yl)-4-((4-(5-(4-(2-(2,6-dioxopiperidin-3-yl)-1-oxoisoindolin-5-yl)piperidin-1-yl)pentanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(7-(4-(2-(2,6-dioxopiperidin-3-yl)-1-oxoisoindolin-5-yl)piperidin-1-yl)heptanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperazin-1-yl)propanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(5-(4-(2-(2,6-dioxopiperidin-3-yl)-3-oxoisoindolin-5-yl)piperazin-1-yl)pentanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(4-(4-(2-(2,6-dioxopiperidin-3-yl)-1-oxoisoindolin-5-yl)piperazin-1-yl)butanoyl)piperazin-1-yl)methyl)piperidin-4-yl)-2,5-difluorobenzamide;
N-[1-(5-{3-cyano-6-ethoxypyrazolo[1,5-a]pyridin-4-yl}pyridin-2-yl)-4-{[4-(3-{4-[2-(2,6-dioxopiperidin-3-yl)-1-oxo-2,3-dihydro-1H-isoindol-5-yl]piperidin-1-yl}propanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(7-{4-[2-(2,6-dioxopiperidin-3-yl)-3-oxo-2,3-dihydro-1H-isoindol-5-yl]piperazin-1-yl}heptanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(3-{4-[2-(2,6-dioxopiperidin-3-yl)-3-oxo-2,3-dihydro-1H-isoindol-5-yl]piperidin-1-yl}propanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(5-{4-[2-(2,6-dioxopiperidin-3-yl)-3-oxo-2,3-dihydro-1H-isoindol-5-yl]piperidin-1-yl}pentanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(7-{4-[2-(2,6-dioxopiperidin-3-yl)-3-oxo-2,3-dihydro-1H-isoindol-5-yl]piperidin-1-yl}heptanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(7-{[4-(2,6-dioxopiperidin-3-yl)phenyl]amino}heptanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(9-{[4-(2,6-dioxopiperidin-3-yl)phenyl]amino}nonanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-({4-[7-({3-[(2,6-dioxopiperidin-3-yl)amino]phenyl}amino)heptanoyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[9-({3-[(2,6-dioxopiperidin-3-yl)amino]phenyl}amino) nonanoyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-{[4-(9-{[3-(2,4-dioxo-1,3-diazinan-1-yl)-1-methyl-1H-indazol-6-yl]amino}nonanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(9-{[3-(2,6-dioxopiperidin-3-yl)-1-methyl-1H-indazol-6-yl]amino}nonanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-({4-[5-(2-{[2-(2,6-dioxopiperidin-3-yl)-1-oxo-2,3-dihydro-1H-isoindol-4-yl]oxy}acetamido)pentanoyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[7-(2-{[2-(2,6-dioxopiperidin-3-yl)-1-oxo-2,3-dihydro-1H-isoindol-4-yl]oxy}acetamido)heptanoyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[11-(2-{[2-(2,6-dioxopiperidin-3-yl)-1-oxo-2,3-dihydro-1H-isoindol-4-yl]oxy}acetamido)undecanoyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[13-(2-{[2-(2,6-dioxopiperidin-3-yl)-1-oxo-2,3-dihydro-1H-isoindol-4-yl]oxy}acetamido)tridecanoyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[15-(2-{[2-(2,6-dioxopiperidin-3-yl)-1-oxo-2,3-dihydro-1H-isoindol-4-yl]oxy}acetamido)pentadecanoyl]piperazin-1-yl}methyl)piperidin-4-yl]-2,5-difluorobenzamide;
N-[1-[5-(3-cyano-6-ethoxy-pyrazolo[1,5-a]pyridin-4-yl)-2-pyridyl]-4-[[2-[4-[4-[2-(2,6-dioxo-3-piperidyl)-1-oxo-isoindolin-5-yl]piperazin-1-yl]butylamino]-2-oxo-ethoxy]methyl]-4-piperidyl]-2,5-difluoro-benzamide;
N-[1-[5-(3-cyano-6-ethoxy-pyrazolo[1,5-a]pyridin-4-yl)-2-pyridyl]-4-[2-[4-[3-[4-[2-(2,6-dioxo-3-piperidyl)-3-oxo-isoindolin-5-yl]-1-piperidyl]propanoyl]piperazin-1-yl]ethoxymethyl]-4-piperidyl]-2,5-difluoro-benzamide;
N-[1-[5-(3-cyano-6-ethoxy-pyrazolo[1,5-a]pyridin-4-yl)-2-pyridyl]-4-[2-[4-[3-[4-[2-(2,6-dioxo-3-piperidyl)-3-oxo-isoindolin-5-yl]piperazin-1-yl]propanoyl]piperazin-1-yl]ethoxymethyl]-4-piperidyl]-2,5-difluoro-benzamide;
N-[1-[5-(3-cyano-6-ethoxy-pyrazolo[1,5-a]pyridin-4-yl)-2-pyridyl]-4-[2-[4-[3-[4-[2-(2,6-dioxo-3-piperidyl)-1-oxo-isoindolin-5-yl]piperazin-1-yl]propanoyl]piperazin-1-yl]ethoxymethyl]-4-piperidyl]-2,5-difluoro-benzamide;
N-[1-[5-(3-cyano-6-ethoxy-pyrazolo[1,5-a]pyridin-4-yl)-2-pyridyl]-4-[2-[4-[5-[4-[2-(2,6-dioxo-3-piperidyl)-1-oxo-isoindolin-5-yl]piperazin-1-yl]pentanoyl]piperazin-1-yl]ethoxymethyl]-4-piperidyl]-2,5-difluoro-benzamide;
N-[1-[5-(3-cyano-6-ethoxy-pyrazolo[1,5-a]pyridin-4-yl)-2-pyridyl]-4-[[2-[4-[4-[2-(2,6-dioxo-3-piperidyl)-1-oxo-isoindolin-5-yl]piperazin-1-yl]methyl]-1-piperidyl]-2-oxo-ethoxy]methyl]-4-piperidyl]-2,5-difluoro-benzamide;
N-[1-[5-(3-cyano-6-ethoxy-pyrazolo[1,5-a]pyridin-4-yl)-2-pyridyl]-4-[2-[4-[3-[4-[2-(2,6-dioxo-3-piperidyl)-3-oxo-isoindolin-5-yl]-1-piperidyl]propyl]piperazin-1-yl]ethoxymethyl]-4-piperidyl]-2,5-difluoro-benzamide;
N-[1-[5-(3-cyano-6-ethoxy-pyrazolo[1,5-a]pyridin-4-yl)-2-pyridyl]-4-[[2-[6-[4-[2-(2,6-dioxo-3-piperidyl)-1-oxo-isoindolin-5-yl]piperazin-1-yl]hexylamino]-2-oxo-ethoxy]methyl]-4-piperidyl]-2,5-difluoro-benzamide;
N-(1-(5-(3-cyano-6-ethoxypyrazolo[1,5-a]pyridin-4-yl)pyridin-2-yl)-4-((4-(3-(4-(3-(2,4-dioxotetrahydropyrimidin-1(2H)-yl)-1-methyl-1H-indazol-6-yl)piperazin-1-yl)propanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(5-(4-(3-(2,4-dioxotetrahydropyrimidin-1(2H)-yl)-1-methyl-1H-indazol-6-yl)piperazin-1-yl)pentanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(7-(4-(3-(2,4-dioxotetrahydropyrimidin-1(2H)-yl)-1-methyl-1H-indazol-6-yl)piperazin-1-yl)heptanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(6-(4-(3-(2,4-dioxotetrahydropyrimidin-1(2H)-yl)-1-methyl-1H-indazol-6-yl)piperidin-1-yl)hexanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(4-(4-(3-(2,4-dioxotetrahydropyrimidin-1(2H)-yl)-1-methyl-1H-indazol-6-yl)piperazin-1-yl)butanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(2-(4-(3-(2,4-dioxotetrahydropyrimidin-1(2H)-yl)-1-methyl-1H-indazol-6-yl)piperidin-1-yl)acetyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(3-(4-(3-(2,4-dioxotetrahydropyrimidin-1(2H)-yl)-1-methyl-1H-indazol-6-yl)piperidin-1-yl)propanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(4-(4-(3-(2,4-dioxotetrahydropyrimidin-1(2H)-yl)-1-methyl-1H-indazol-6-yl)piperidin-1-yl)butanoyl)piperazin-1-yl)methyl)piperidin-4-yl)-2,5-difluorobenzamide;
N-[1-[5-(3-cyano-6-ethoxy-pyrazolo[1,5-a]pyridin-4-yl)-2-pyridyl]-4-[[1-[6-[4-[2-(2,6-dioxo-3-piperidyl)-1-oxo-isoindolin-5-yl]piperazin-1-yl]hexyl]triazol-4-yl]methoxymethyl]-4-piperidyl]-2,5-difluoro-benzamide;
N-[1-[5-(3-cyano-6-ethoxy-pyrazolo[1,5-a]pyridin-4-yl)-2-pyridyl]-4-[1-[4-[4-[2-(2,6-dioxo-3-piperidyl)-1-oxo-isoindolin-5-yl]piperazin-1-yl]butyl]triazol-4-yl]methoxymethyl]-4-piperidyl]-2,5-difluoro-benzamide;
N-[1-[5-(3-cyano-6-ethoxy-pyrazolo[1,5-a]pyridin-4-yl)-2-pyridyl]-4-[[1-[2-[4-[2-(2,6-dioxo-3-piperidyl)-1-oxo-isoindolin-5-yl]piperazin-1-yl]ethyl]triazol-4-yl]methoxymethyl]-4-piperidyl]-2,5-difluoro-benzamide;
N-(1-(5-(3-cyano-6-ethoxypyrazolo[1,5-a]pyridin-4-yl)pyridin-2-yl)-4-(((1-(3-(4-(2-(2,6-dioxopiperidin-3-yl)-1-oxoisoindolin-5-yl)piperazin-1-yl) propyl)-1H-1,2,3-triazol-4-yl) methoxy)methyl)piperidin-4-yl)-2,5-difluorobenzamide;
N-[4-benzyl-1-[5-(3-cyano-6-ethoxy-pyrazolo[1,5-a]pyridin-4-yl)-2-pyridyl]-4-piperidyl]-4-[3-[4-[2-(2,6-dioxo-3-piperidyl)-3-oxo-isoindolin-5-yl]-1-piperidyl]propanoyl]piperazine-1-carboxamide;
N-[4-benzyl-1-[5-(3-cyano-6-ethoxy-pyrazolo[1,5-a]pyridin-4-yl)-2-pyridyl]-4-piperidyl]-1-[3-[4-[2-(2,6-dioxo-3-piperidyl)-3-oxo-isoindolin-5-yl]-1-piperidyl]propanoyl]piperidine-4-carboxamide;
N-[4-benzyl-1-[5-(3-cyano-6-ethoxy-pyrazolo[1,5-a]pyridin-4-yl)-2-pyridyl]-4-piperidyl]-2-[4-[3-[4-[2-(2,6-dioxo-3-piperidyl)-3-oxo-isoindolin-5-yl]-1-piperidyl]propanoyl]piperazin-1-yl]acetamide;
N-[4-benzyl-1-[5-(3-cyano-6-ethoxy-pyrazolo[1,5-a]pyridin-4-yl)-2-pyridyl]-4-piperidyl]-2-[1-[3-[4-[2-(2,6-dioxo-3-piperidyl)-3-oxo-isoindolin-5-yl]-1-piperidyl]propanoyl]-4-piperidyl]acetamide;
N-[4-benzyl-1-[5-(3-cyano-6-ethoxy-pyrazolo[1,5-apyridin-4-yl)-2-pyridyl]-4-piperidyl]-4-[[4-[2-(2,6-dioxo-3-piperidyl)-3-oxo-isoindolin-5-yl]-1-piperidyl]methyl]piperidine-1-carboxamide;
N-[4-benzyl-1-[5-(3-cyano-6-ethoxy-pyrazolo[1,5-a]pyridin-4-yl)-2-pyridyl]-4-piperidyl]-4-[4-[2-(2,6-dioxo-3-piperidyl)-3-oxo-isoindolin-5-yl]-1-piperidyl]piperidine-1-carboxamide;
N-[4-benzyl-1-[5-(3-cyano-6-ethoxy-pyrazolo[1,5-a]pyridin-4-yl)-2-pyridyl]-4-piperidyl]-2-[4-[[4-[2-(2,6-dioxo-3-piperidyl)-3-oxo-isoindolin-5-yl]-1-piperidyl]methyl]-1-piperidyl]acetamide;
N-(1-(5-(3-cyano-6-ethoxypyrazolo[1,5-a]pyridin-4-yl)pyridin-2-yl)-4-((4-(3-(4-(4-((2,6-dioxopiperidin-3-yl)amino)phenyl)piperazin-1-yl)propanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(3-(4-(3-((2,6-dioxopiperidin-3-yl)amino)phenyl)piperazin-1-yl)propanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(3-(4-(4-(2,6-dioxopiperidin-3-yl)phenyl)piperazin-1-yl)propanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(3-(4-(3-(2,6-dioxopiperidin-3-yl)phenyl)piperazin-1-yl)propanoyl)piperazin-1-yl)methyl)piperidin-4-yl)-2,5-difluorobenzamide;
N-[1-(5-{3-cyano-6-ethoxypyrazolo[1,5-a]pyridin-4-yl}pyridin-2-yl)-4-{[4-(1-{2-[2-(2,6-dioxopiperidin-3-yl)-1-oxo-2,3-dihydro-1H-isoindol-5-yl]acetyl}azetidin-3-yl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-[(4-{1-[2-(2,6-dioxopiperidin-3-yl)-1-oxo-2,3-dihydro-1H-isoindole-5-carbonyl]azetidin-3-yl}piperazin-1-yl)methyl]piperidin-4-yl]-2,5-difluorobenzamide;
N-[1-(5-{3-cyano-6-ethoxypyrazolo[1,5-a]pyridin-4-yl}pyridin-2-yl)-4-[(4-{1-[3-(2,4-dioxo-1,3-diazinan-1-yl)-1-methyl-1H-indazole-6-carbonyl]azetidin-3-yl}piperazin-1-yl)methyl]piperidin-4-yl]-2,5-difluorobenzamide;
N-[1-(5-{3-cyano-6-ethoxypyrazolo[1,5-a]pyridin-4-yl}pyridin-2-yl)-4-({4-[1-(2-{[2-(2,6-dioxopiperidin-3-yl)-1-oxo-2,3-dihydro-1H-isoindol-5-yl]oxy}acetyl)azetidin-3-yl]piperazin-1-yl}methyl)piperidin-4-yl]-2,5-difluorobenzamide;
N-[4-benzyl-1-(5-{3-cyano-6-ethoxypyrazolo[1,5-a]pyridin-4-yl}pyridin-2-yl)piperidin-4-yl]-1-(3-{4-[2-(2,6-dioxopiperidin-3-yl)-1-oxo-2,3-dihydro-1H-isoindol-5-yl]piperidin-1-yl}propanoyl)piperidine-4-carboxamide;
N-(1-(5-(3-cyano-6-ethoxypyrazolo[1,5-a]pyridin-4-yl)pyridin-2-yl)-4-((4-((4-(2-(2,6-dioxopiperidin-3-yl)-3-oxoisoindolin-5-yl)piperazin-1-yl)methyl)piperidin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(4-(2-(2,6-dioxopiperidin-3-yl)-3-oxoisoindolin-5-yl)piperazin-1-yl)piperidin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-((4-(2-(2,6-dioxopiperidin-3-yl)-1-oxoisoindolin-5-yl)piperazin-1-yl)methyl)piperidin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)azetidine-1-carbonyl)piperidin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)azetidin-1-yl)piperidin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-((3-(4-(2-(2,6-dioxopiperidin-3-yl)-3-oxoisoindolin-5-yl)piperidin-1-yl)azetidin-1-yl)methyl)piperidin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)propanoyl)piperazin-1-yl)methyl)piperidin-4-yl)-1-fluorocyclopropane-1-carboxamide;
N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)cyclobutane-1-carbonyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(1-(1-(2-(2,6-dioxopiperidin-3-yl)-3-oxoisoindolin-5-yl)piperidin-4-yl)azetidin-3-yl)piperazin-1-yl)methyl)piperidin-4-yl)-2,5-difluorobenzamide;
1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)propanoyl)piperazin-1-yl)methyl)-N-isopropylpiperidine-4-carboxamide;
1-(5-(3-Cyano-6-ethoxypyrazolo[1,5-a]pyridin-4-yl)pyridin-2-yl)-4-((4-(3-(4-(2-(2,6-dioxopiperidin-3-yl)-1-oxoisoindolin-5-yl)piperazin-1-yl)propanoyl)piperazin-1-yl)methyl)-N-isopropylpiperidine-4-carboxamide;
1-(5-{3-cyano-6-ethoxypyrazolo[1,5-a]pyridin-4-yl}pyridin-2-yl)-4-{[4-(3-{4-[2-(2,6-dioxopiperidin-3-yl)-3-oxo-2,3-dihydro-1H-isoindol-5-yl]piperidin-1-yl}propanoyl)piperazin-1-yl]methyl}-N-(2-methylpropyl)piperidine-4-carboxamide;
N-(1-(5-(3-cyano-6-ethoxypyrazolo[1,5-a]pyridin-4-yl)pyridin-2-yl)-4-((4-(1-(3-(2,4-dioxotetrahydropyrimidin-1(2H)-yl)-1-methyl-1H-indazol-6-yl)piperidine-4-carbonyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(3-(4-(2-(3-methyl-2,6-dioxopiperidin-3-yl)-3-oxoisoindolin-5-yl)piperidin-1-yl)propanoyl)piperazin-1-yl)methyl)piperidin-4-yl)-2,5-difluorobenzamide;
(S)—N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)propanoyl)piperazin-1-yl)methyl)piperidin-4-yl)-2,5-difluorobenzamide;
(R)—N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)propanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(6-(4-(2-(2,6-dioxopiperidin-3-yl)-1-oxoisoindolin-5-yl)piperazin-1-yl)spiro[3.3]heptane-2-carbonyl)piperazin-1-yl)methyl)piperidin-4-yl)-2,5-difluorobenzamide;
6-(4-{[1-(5-{3-cyano-6-ethoxypyrazolo[1,5-a]pyridin-4-yl}pyridin-2-yl)-4-(2,5-difluorobenzamido)piperidin-4-yl]methyl}piperazine-1-carbonyl)-N-[3-(2,4-dioxo-1,3-diazinan-1-yl)-1-methyl-1H-indazol-6-yl]spiro[3.3]heptane-2-carboxamide;
N-[1-(5-{3-cyano-6-ethoxypyrazolo[1,5-a]pyridin-4-yl}pyridin-2-yl)-4-{[4-(6-{4-[2-(2,6-dioxopiperidin-3-yl)-1-oxo-2,3-dihydro-1H-isoindol-5-yl]piperazine-1-carbonyl}spiro[3.3]heptane-2-carbonyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(6-{4-[2-(2,6-dioxopiperidin-3-yl)-3-oxo-2,3-dihydro-1H-isoindol-5-yl]piperazine-1-carbonyl}spiro[3.3]heptane-2-carbonyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[9-(3-{4-[2-(2,6-dioxopiperidin-3-yl)-1-oxo-2,3-dihydro-1H-isoindol-5-yl]piperazin-1-yl}propanoyl)-3,9-diazaspiro[5.5]undecan-3-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[9-(3-{4-[2-(2,6-dioxopiperidin-3-yl)-1-oxo-2,3-dihydro-1H-isoindol-5-yl]piperidin-1-yl}propanoyl)-3,9-diazaspiro[5.5]undecan-3-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[6-(3-{4-[2-(2,6-dioxopiperidin-3-yl)-1-oxo-2,3-dihydro-1H-isoindol-5-yl]piperidin-1-yl}propanoyl)-2,6-diazaspiro[3.3]heptan-2-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(3-{9-[2-(2,6-dioxopiperidin-3-yl)-1-oxo-2,3-dihydro-1H-isoindol-5-yl]-3,9-diazaspiro[5.5]undecan-3-yl}-3-oxopropyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(3-{9-[2-(2,6-dioxopiperidin-3-yl)-1-oxo-2,3-dihydro-1H-isoindol-5-yl]-3,9-diazaspiro[5.5]undecan-3-yl}propanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(6-{4-[2-(2,6-dioxopiperidin-3-yl)-1-oxo-2,3-dihydro-1H-isoindol-5-yl]piperidin-1-yl}spiro[3.3]heptane-2-carbonyl)piperazin-1-yl]methyl}piperidin-4-yl]-2,5-difluorobenzamide
N-[1-(5-{3-cyano-6-ethoxypyrazolo[1,5-a]pyridin-4-yl}pyridin-2-yl)-4-{[4-(6-{[3-(2,4-dioxo-1,3-diazinan-1-yl)-1-methyl-1H-indazol-6-yl]amino}spiro[3.3]heptane-2-carbonyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(6-{4-[2-(2,6-dioxopiperidin-3-yl)-1-oxo-2,3-dihydro-1H-isoindol-5-yl]piperazine-1-carbonyl}spiro[3.3]heptan-2-yl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(6-{4-[2-(2,6-dioxopiperidin-3-yl)-1-oxo-2,3-dihydro-1H-isoindol-5-yl]piperidine-1-carbonyl}spiro[3.3]heptan-2-yl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-({9-[2-(2,6-dioxopiperidin-3-yl)-3-oxo-2,3-dihydro-1H-isoindole-5-carbonyl]-3,9-diazaspiro[5.5]undecan-3-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-{[6-(2-{[2-(2,6-dioxopiperidin-3-yl)-1-oxo-2,3-dihydro-1H-isoindol-5-yl]oxy}acetyl)-2,6-diazaspiro[3.3]heptan-2-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-[(9-{2-[2-(2,6-dioxopiperidin-3-yl)-1-oxo-2,3-dihydro-1H-isoindol-5-yl]acetyl}-3,9-diazaspiro[5.5]undecan-3-yl)methyl]piperidin-4-yl]-2,5-difluorobenzamide;
N-[1-(5-{3-cyano-6-ethoxypyrazolo[1,5-a]pyridin-4-yl}pyridin-2-yl)-4-{[9-(2-{[2-(2,6-dioxopiperidin-3-yl)-1-oxo-2,3-dihydro-1H-isoindol-5-yl]oxy}acetyl)-3,9-diazaspiro[5.5]undecan-3-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-({4-[2-(3-{4-[2-(2,6-dioxopiperidin-3-yl)-1-oxo-2,3-dihydro-1H-isoindol-5-yl]piperazin-1-yl}-3-oxopropoxy)ethyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[2-(3-{4-[2-(2,6-dioxopiperidin-3-yl)-3-oxo-2,3-dihydro-1H-isoindol-5-yl]piperidin-1-yl}-3-oxopropoxy)ethyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[2-(2-{[3-(2,4-dioxo-1,3-diazinan-1-yl)-1-methyl-1H-indazol-6-yl]carbamoyl}ethoxy)ethyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[2-(3-{4-[2-(2,6-dioxopiperidin-3-yl)-3-oxo-2,3-dihydro-1H-isoindol-5-yl]piperazin-1-yl}-3-oxopropoxy)ethyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[3-(2-{4-[2-(2,6-dioxopiperidin-3-yl)-1-oxo-2,3-dihydro-1H-isoindol-5-yl]piperazin-1-yl}ethoxy)propanoyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[3-(2-{[3-(2,4-dioxo-1,3-diazinan-1-yl)-1-methyl-1H-indazol-6-yl]amino}ethoxy)propanoyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-[(4-{2-[2-(2,6dioxopiperidin-3-yl)-3-oxo-2,3-dihydro-1H-isoindol-5-yl]acetyl}piperazin-1-yl)methyl]piperidin-4-yl]-2,5-difluorobenzamide;
N-[1-(5-{3-cyano-6-ethoxypyrazolo[1,5-a]pyridin-4-yl}pyridin-2-yl)-4-{[4-(2-{[2-(2,6-dioxopiperidin-3-yl)-1-oxo-2,3-dihydro-1H-isoindol-5-yl]oxy}acetyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-[(4-{2-[2-(2-{[3-(2,4-dioxo-1,3-diazinan-1-yl)-1-methyl-1H-indazol-6-yl]carbamoyl}ethoxy)ethoxy]ethyl}piperazin-1-yl)methyl]piperidin-4-yl]-2,5-difluorobenzamide;
N-[1-(5-{3-cyano-6-ethoxypyrazolo[1,5-a]pyridin-4-yl}pyridin-2-yl)-4-[(4-{2-[2-(3-{4-[2-(2,6-dioxopiperidin-3-yl)-1-oxo-2,3-dihydro-1H-isoindol-5-yl]piperazin-1-yl}-3-oxopropoxy)ethoxy]ethyl}piperazin-1-yl)methyl]piperidin-4-yl]-2,5-difluorobenzamide;
N-[1-(5-{3-cyano-6-ethoxypyrazolo[1,5-a]pyridin-4-yl}pyridin-2-yl)-4-{[4-({1-[2-(2,6-dioxopiperidin-3-yl)-3-oxo-2,3-dihydro-1H-isoindole-5-carbonyl]piperidin-4-yl}methyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-({4-[(1-{2-[2-(2,6-dioxopiperidin-3-yl)-1-oxo-2,3-dihydro-1H-isoindol-4-yl]acetyl}piperidin-4-yl)methyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[(1-{2-[2-(2,6-dioxopiperidin-3-yl)-1-oxo-2,3-dihydro-1H-isoindol-5-yl]acetyl}piperidin-4-yl)methyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[(1-{2-[2-(2,6-dioxopiperidin-3-yl)-3-oxo-2,3-dihydro-1H-isoindol-5-yl]acetyl}piperidin-4-yl)methyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-[(4-{[1-(2-{[2-(2,6-dioxopiperidin-3-yl)-1-oxo-2,3-dihydro-1H-isoindol-5-yl]oxy}acetyl)piperidin-4-yl]methyl}piperazin-1-yl)methyl]piperidin-4-yl]-2,5-difluorobenzamide;
N-[1-(5-{3-cyano-6-ethoxypyrazolo[1,5-a]pyridin-4-yl}pyridin-2-yl)-4-{[4-(3-{4-[2-(2,6-dioxopiperidin-3-yl)-3-oxo-2,3-dihydro-1H-isoindol-5-yl]piperazin-1-yl}-3-oxopropyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(3-{4-[2-(2,6-dioxopiperidin-3-yl)-3-oxo-2,3-dihydro-1H-isoindol-5-yl]piperidin-1-yl}-3-oxopropyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(3-{4-[2-(2,6-dioxopiperidin-3-yl)-1,3-dioxo-2,3-dihydro-1H-isoindol-5-yl]piperidin-1-yl}-3-oxopropyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-({4-[3-(4-{[2-(2,6-dioxopiperidin-3-yl)-1-oxo-2,3-dihydro-1H-isoindol-5-yl]oxy}piperidin-1-yl)propanoyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-{[4-(3-{3-[2-(2,6-dioxopiperidin-3-yl)-1-oxo-2,3-dihydro-1H-isoindol-5-yl]azetidin-1-yl}propanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-({4-[3-(3-{[2-(2,6-dioxopiperidin-3-yl)-1-oxo-2,3-dihydro-1H-isoindol-5-yl]oxy}azetidin-1-yl)propanoyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-{[9-(2-{4-[2-(2,6-dioxopiperidin-3-yl)-3-oxo-2,3-dihydro-1H-isoindol-5-yl]piperidin-1-yl}-2-oxoethyl)-3-azaspiro[5.5]undecan-3-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[9-(2-{4-[2-(2,6-dioxopiperidin-3-yl)-1-oxo-2,3-dihydro-1H-isoindol-5-yl]piperidin-1-yl}-2-oxoethyl)-3-azaspiro[5.5]undecan-3-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[9-(2-{4-[2-(2,6-dioxopiperidin-3-yl)-3-oxo-2,3-dihydro-1H-isoindol-5-yl]piperazin-1-yl}-2-oxoethyl)-3-azaspiro[5.5]undecan-3-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[9-(2-{4-[2-(2,6-dioxopiperidin-3-yl)-1-oxo-2,3-dihydro-1H-isoindol-5-yl]piperazin-1-yl}-2-oxoethyl)-3-azaspiro[5.5]undecan-3-yl]methyl}piperidin-4-yl]-2,5-difluorobenzamide; 4-[6-(4-{[4-(7-{4-[2-(2,6-dioxopiperidin-3-yl)-1-oxo-2,3-dihydro-1H-isoindol-5-yl]piperazin-1-yl}heptanoyl)piperazin-1-yl]methyl}piperidin-1-yl)pyridin-3-yl]-6-ethoxypyrazolo[1,5-a]pyridine-3-carbonitrile;
4-[6-(4-{[4-(3-{4-[2-(2,6-dioxopiperidin-3-yl)-3-oxo-2,3-dihydro-1H-isoindol-5-yl]piperidin-1-yl}propanoyl)piperazin-1-yl]methyl}piperidin-1-yl)pyridin-3-yl]-6-ethoxypyrazolo[1,5-a]pyridine-3-carbonitrile;
N-[1-(5-{3-cyano-6-ethoxypyrazolo[1,5-a]pyridin-4-yl}pyridin-2-yl)-4-[(methylamino)methyl]piperidin-4-yl]-2,5-difluorobenzamide;
4-[6-(4-{[4-(7-{4-[2-(2,6-dioxopiperidin-3-yl)-1-oxo-2,3-dihydro-1H-isoindol-5-yl]piperazin-1-yl}heptanoyl)piperazin-1-yl]methyl}-4-methylpiperidin-1-yl)pyridin-3-yl]-6-ethoxypyrazolo[1,5-a]pyridine-3-carbonitrile;
4-[6-(4-{[4-(3-{4-[2-(2,6-dioxopiperidin-3-yl)-1-oxo-2,3-dihydro-1H-isoindol-5-yl]piperidin-1-yl}propanoyl)piperazin-1-yl]methyl}-4-methylpiperidin-1-yl)pyridin-3-yl]-6-ethoxypyrazolo[1,5-a]pyridine-3-carbonitrile;
4-[6-(4-{[4-(7-{4-[2-(2,6-dioxopiperidin-3-yl)-1-oxo-2,3-dihydro-1H-isoindol-5-yl]piperazin-1-yl}heptanoyl)piperazin-1-yl]methyl}-4-ethylpiperidin-1-yl)pyridin-3-yl]-6-ethoxypyrazolo[1,5-a]pyridine-3-carbonitrile;
4-[6-(4-{[4-(3-{4-[2-(2,6-dioxopiperidin-3-yl)-1-oxo-2,3-dihydro-1H-isoindol-5-yl]piperidin-1-yl}propanoyl)piperazin-1-yl]methyl}-4-ethylpiperidin-1-yl)pyridin-3-yl]-6-ethoxypyrazolo[1,5-a]pyridine-3-carbonitrile;
4-[6-(4-{[4-(3-{4-[2-(2,6-dioxopiperidin-3-yl)-3-oxo-2,3-dihydro-1H-isoindol-5-yl]piperidin-1-yl}propanoyl)piperazin-1-yl]methyl}-4-ethylpiperidin-1-yl)pyridin-3-yl]-6-ethoxypyrazolo[1,5-a]pyridine-3-carbonitrile;
4-[6-(4-{[4-(2-{[2-(2,6-dioxopiperidin-3-yl)-1-oxo-2,3-dihydro-1H-isoindol-5-yl]oxy}acetyl)piperazin-1-yl]methyl}-4-ethylpiperidin-1-yl)pyridin-3-yl]-6-ethoxypyrazolo[1,5-a]pyridine-3-carbonitrile;
N-(1-(5-(3-cyano-6-ethoxypyrazolo[1,5-a]pyridin-4-yl)pyridin-2-yl)-4-(9-(2-(2,6-dioxopiperidin-3-yl)-1-oxoisoindolin-5-yl)-3,9-diazaspiro[5.5]undecane-3-carbonyl)piperidin-4-yl)-2,5-difluorobenzamide;
N-[1-(5-{3-cyano-6-ethoxypyrazolo[1,5-a]pyridin-4-yl}pyridin-2-yl)-4-{[4-(7-{4-[2-(1-methyl-2,6-dioxopiperidin-3-yl)-1-oxo-2,3-dihydro-1H-isoindol-5-yl]piperazin-1-yl}heptanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-[(5-{4-[2-(2,6-dioxopiperidin-3-yl)-1-oxo-2,3-dihydro-1H-isoindol-5-yl]piperazin-1-yl}-N-methylpentanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(7-{4-[2-(2,6-dioxopiperidin-3-yl)-1-oxo-2,3-dihydro-1H-isoindol-5-yl]piperazin-1-yl}-N-methylheptanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(3-{4-[2-(2,6-dioxopiperidin-3-yl)-1-oxo-2,3-dihydro-1H-isoindol-5-yl]piperidin-1-yl}-N-methylpropanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(5-{4-[2-(2,6-dioxopiperidin-3-yl)-1-oxo-2,3-dihydro-1H-isoindol-5-yl]piperidin-1-yl}-N-methylpentanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(7-{4-[2-(2,6-dioxopiperidin-3-yl)-1-oxo-2,3-dihydro-1H-isoindol-5-yl]piperidin-1-yl}-N-methylheptanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(3-{4-[2-(2,6-dioxopiperidin-3-yl)-3-oxo-2,3-dihydro-1H-isoindol-5-yl]piperazin-1-yl}-N-methylpropanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(5-{4-[2-(2,6-dioxopiperidin-3-yl)-3-oxo-2,3-dihydro-1H-isoindol-5-yl]piperazin-1-yl}-N-methylpentanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(7-{4-[2-(2,6-dioxopiperidin-3-yl)-3-oxo-2,3-dihydro-1H-isoindol-5-yl]piperazin-1-yl}-N-methylheptanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(3-{4-[2-(2,6-dioxopiperidin-3-yl)-3-oxo-2,3-dihydro-1H-isoindol-5-yl]piperidin-1-yl}-N-methylpropanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(5-{4-[2-(2,6-dioxopiperidin-3-yl)-3-oxo-2,3-dihydro-1H-isoindol-5-yl]piperidin-1-yl}-N-methylpentanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(7-{4-[2-(2,6-dioxopiperidin-3-yl)-3-oxo-2,3-dihydro-1H-isoindol-5-yl]piperidin-1-yl}-N-methylheptanamido)methyl]piperidin-4-yl]-2,5-difluorobenzamide;
4-[6-(4-{[4-(3-{4-[2-(2,6-dioxopiperidin-3-yl)-3-oxo-2,3-dihydro-1H-isoindol-5-yl]piperidin-1-yl}propanoyl)piperazin-1-yl]methyl}-4-methylpiperidin-1-yl)pyridin-3-yl]-6-ethoxypyrazolo[1,5-a]pyridine-3-carbonitrile;
1-(5-{3-cyano-6-ethoxypyrazolo[1,5-a]pyridin-4-yl}pyridin-2-yl)-4-(2,5-difluorobenzamido)-N-(6-{[4-(2,4-dioxo-1,3-diazinan-1-yl)phenyl]amino}hexyl)piperidine-4-carboxamide;
1-(5-{3-cyano-6-ethoxypyrazolo[1,5-a]pyridin-4-yl}pyridin-2-yl)-4-(2,5-difluorobenzamido)-N-(6-{[3-(2,4-dioxo-1,3-diazinan-1-yl)-1-methyl-1H-indazol-7-yl]amino}hexyl)piperidine-4-carboxamide;
1-(5-{3-cyano-6-ethoxypyrazolo[1,5-a]pyridin-4-yl}pyridin-2-yl)-4-(2,5-difluorobenzamido)-N-(10-{[3-(2,4-dioxo-1,3-diazinan-1-yl)-1-methyl-1H-indazol-7-yl]amino}decyl)piperidine-4-carboxamide;
1-(5-{3-cyano-6-ethoxypyrazolo[1,5-a]pyridin-4-yl}pyridin-2-yl)-4-(2,5-difluorobenzamido)-N-(6-{[3-(2,4-dioxo-1,3-diazinan-1-yl)-1-methyl-1H-indazol-6-yl]amino}hexyl)piperidine-4-carboxamide;
1-(5-{3-cyano-6-ethoxypyrazolo[1,5-a]pyridin-4-yl}pyridin-2-yl)-4-(2,5-difluorobenzamido)-N-(8-{[3-(2,4-dioxo-1,3-diazinan-1-yl)-1-methyl-1H-indazol-6-yl]amino}octyl)piperidine-4-carboxamide;
1-(5-{3-cyano-6-ethoxypyrazolo[1,5-a]pyridin-4-yl}pyridin-2-yl)-4-(2,5-difluorobenzamido)-N-(10-{[3-(2,4-dioxo-1,3-diazinan-1-yl)-1-methyl-1H-indazol-6-yl]amino}decyl)piperidine-4-carboxamide;
1-(5-{3-cyano-6-ethoxypyrazolo[1,5-a]pyridin-4-yl}pyridin-2-yl)-4-(2,5-difluorobenzamido)-N-(4-{4-[2-(2,6-dioxopiperidin-3-yl)-1-oxo-2,3-dihydro-1H-isoindol-5-yl]piperazin-1-yl}butyl)piperidine-4-carboxamide;
1-(5-{3-cyano-6-ethoxypyrazolo[1,5-a]pyridin-4-yl}pyridin-2-yl)-4-(2,5-difluorobenzamido)-N-(6-{4-[2-(2,6-dioxopiperidin-3-yl)-1-oxo-2,3-dihydro-1H-isoindol-5-yl]piperazin-1-yl}hexyl)piperidine-4-carboxamide;
1-(5-{3-cyano-6-ethoxypyrazolo[1,5-a]pyridin-4-yl}pyridin-2-yl)-4-(2,5-difluorobenzamido)-N-(2-{4-[2-(2,6-dioxopiperidin-3-yl)-1-oxo-2,3-dihydro-1H-isoindol-5-yl]piperidin-1-yl}ethyl)piperidine-4-carboxamide;
1-(5-{3-cyano-6-ethoxypyrazolo[1,5-a]pyridin-4-yl}pyridin-2-yl)-4-(2,5-difluorobenzamido)-N-(6-{4-[2-(2,6-dioxopiperidin-3-yl)-1-oxo-2,3-dihydro-1H-isoindol-5-yl]piperidin-1-yl}hexyl)piperidine-4-carboxamide;
1-(5-{3-cyano-6-ethoxypyrazolo[1,5-a]pyridin-4-yl}pyridin-2-yl)-4-(2,5-difluorobenzamido)-N-(2-{4-[2-(2,6-dioxopiperidin-3-yl)-3-oxo-2,3-dihydro-1H-isoindol-5-yl]piperazin-1-yl}ethyl)piperidine-4-carboxamide;
1-(5-{3-cyano-6-ethoxypyrazolo[1,5-a]pyridin-4-yl}pyridin-2-yl)-4-(2,5-difluorobenzamido)-N-(4-{4-[2-(2,6-dioxopiperidin-3-yl)-3-oxo-2,3-dihydro-1H-isoindol-5-yl]piperazin-1-yl}butyl)piperidine-4-carboxamide;
1-(5-{3-cyano-6-ethoxypyrazolo[1,5-a]pyridin-4-yl}pyridin-2-yl)-4-(2,5-difluorobenzamido)-N-(6-{4-[2-(2,6-dioxopiperidin-3-yl)-3-oxo-2,3-dihydro-1H-isoindol-5-yl]piperazin-1-yl}hexyl)piperidine-4-carboxamide;
1-(5-{3-cyano-6-ethoxypyrazolo[1,5-a]pyridin-4-yl}pyridin-2-yl)-4-(2,5-difluorobenzamido)-N-(2-{4-[2-(2,6-dioxopiperidin-3-yl)-3-oxo-2,3-dihydro-1H-isoindol-5-yl]piperidin-1-yl}ethyl)piperidine-4-carboxamide;
1-(5-{3-cyano-6-ethoxypyrazolo[1,5-a]pyridin-4-yl}pyridin-2-yl)-4-(2,5-difluorobenzamido)-N-(4-{4-[2-(2,6-dioxopiperidin-3-yl)-3-oxo-2,3-dihydro-1H-isoindol-5-yl]piperidin-1-yl}butyl)piperidine-4-carboxamide;
1-(5-{3-cyano-6-ethoxypyrazolo[1,5-a]pyridin-4-yl}pyridin-2-yl)-4-(2,5-difluorobenzamido)-N-(6-{4-[2-(2,6-dioxopiperidin-3-yl)-3-oxo-2,3-dihydro-1H-isoindol-5-yl]piperidin-1-yl}hexyl)piperidine-4-carboxamide;
N-[1-(5-{3-cyano-6-ethoxypyrazolo[1,5-a]pyridin-4-yl}pyridin-2-yl)-4-[(5-{4-[2-(2,6-dioxopiperidin-3-yl)-1-oxo-2,3-dihydro-1H-isoindol-5-yl]piperazin-1-yl}-N-(propan-2-yl)pentanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(7-{4-[2-(2,6-dioxopiperidin-3-yl)-1-oxo-2,3-dihydro-1H-isoindol-5-yl]piperazin-1-yl}-N-(propan-2-yl)heptanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(7-{4-[2-(2,6-dioxopiperidin-3-yl)-1-oxo-2,3-dihydro-1H-isoindol-5-yl]piperidin-1-yl}-N-(propan-2-yl)heptanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(3-{4-[2-(2,6-dioxopiperidin-3-yl)-3-oxo-2,3-dihydro-1H-isoindol-5-yl]piperazin-1-yl}-N-(propan-2-yl)propanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(5-{4-[2-(2,6-dioxopiperidin-3-yl)-3-oxo-2,3-dihydro-1H-isoindol-5-yl]piperazin-1-yl}-N-(propan-2-yl)pentanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(7-{4-[2-(2,6-dioxopiperidin-3-yl)-3-oxo-2,3-dihydro-1H-isoindol-5-yl]piperazin-1-yl}-N-(propan-2-yl)heptanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(5-{4-[2-(2,6-dioxopiperidin-3-yl)-3-oxo-2,3-dihydro-1H-isoindol-5-yl]piperidin-1-yl}-N-(propan-2-yl)pentanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[9-(2-{4-[2-(2,6-dioxopiperidin-3-yl)-3-oxo-2,3-dihydro-1H-isoindol-5-yl]piperidin-1-yl}-2-oxoethyl)-3-azaspiro[5.5]undecane-3-carbonyl]piperidin-4-yl]-2,5-difluorobenzamide;
N-[1-(5-{3-cyano-6-ethoxypyrazolo[1,5-a]pyridin-4-yl}pyridin-2-yl)-4-[9-(2-{4-[2-(2,6-dioxopiperidin-3-yl)-1-oxo-2,3-dihydro-1H-isoindol-5-yl]piperidin-1-yl}-2-oxoethyl)-3-azaspiro[5.5]undecane-3-carbonyl]piperidin-4-yl]-2,5-difluorobenzamide;
N-[1-(5-{3-cyano-6-ethoxypyrazolo[1,5-a]pyridin-4-yl}pyridin-2-yl)-4-[9-(2-{4-[2-(2,6-dioxopiperidin-3-yl)-3-oxo-2,3-dihydro-1H-isoindol-5-yl]piperazin-1-yl}-2-oxoethyl)-3-azaspiro[5.5]undecane-3-carbonyl]piperidin-4-yl]-2,5-difluorobenzamide;
N-[1-(5-{3-cyano-6-ethoxypyrazolo[1,5-a]pyridin-4-yl}pyridin-2-yl)-4-[9-(2-{4-[2-(2,6-dioxopiperidin-3-yl)-1-oxo-2,3-dihydro-1H-isoindol-5-yl]piperazin-1-yl}-2-oxoethyl)-3-azaspiro[5.5]undecane-3-carbonyl]piperidin-4-yl]-2,5-difluorobenzamide;
(S)—N-(1-(5-(3-cyano-6-ethoxypyrazolo[1,5-a]pyridin-4-yl)pyridin-2-yl)-4-((4-(7-(4-(2-(2,6-dioxopiperidin-3-yl)-3-oxoisoindolin-5-yl)piperazin-1-yl)heptanoyl)piperazin-1-yl)methyl)piperidin-4-yl)-2,5-difluorobenzamide;
(R)—N-(1-(5-(3-cyano-6-ethoxypyrazolo[1,5-a]pyridin-4-yl)pyridin-2-yl)-4-((4-(7-(4-(2-(2,6-dioxopiperidin-3-yl)-3-oxoisoindolin-5-yl)piperazin-1-yl)heptanoyl)piperazin-1-yl)methyl)piperidin-4-yl)-2,5-difluorobenzamide; or
N-[1-(5-{3-cyano-6-ethoxypyrazolo[1,5-a]pyridin-4-yl}pyridin-2-yl)-4-[(7-{4-[2-(2,6-dioxopiperidin-3-yl)-3-oxo-2,3-dihydro-1H-isoindol-5-yl]piperidin-1-yl}-N-(propan-2-yl)heptanamido)methyl]piperidin-4-yl]-2,5-difluorobenzamide.

### Embodiment 19

A compound of Formula II:

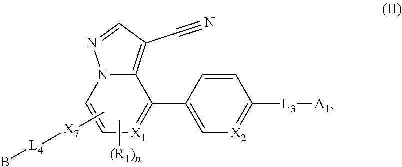

and pharmaceutically acceptable salts, solvates, isomers, enantiomers, and tautomers thereof,
wherein
R₁ is —OCH₂CH₃, —OCH₃, —OCF₃, —OCH₂CH₂OH, —OCH₂CH₂F, 1-methylpyrazol-4-yl, or halogen;
A₁ is independently aryl, or heteroaryl, wherein the aryl or heteroaryl is optionally substituted with one or more R₂; wherein R₂ is independently halogen, OH, C₁-C₃ alkyl, or C₁-C₃ alkoxy;
B is a moiety that is capable of binding a E3 ligase;
L₄ is independently —(CH₂)ₚ-(4- to 12-heterocycloalkyl)-Y₁—C(O)—Y₂—, —(CH₂)ₚ-(4- to 12-heterocycloalkyl)-C(O)—Y₁-Y₂—, —(CH₂)ₚ-(4- to 12-heterocycloalkyl)-Y₁-Y₂—,
   wherein
      Y₁ is independently —C₁₋₈ alkylenyl, 4- to 12-membered heterocycloalkyl, or —C₃-C₁₀ cycloalkyl;
      Y₂ is independently
         —C₁-C₃ alkyl-,
         —(CH₂)ₘ—O—,
         —O—(CH₂)ₘ-(4- to 6-membered heterocycloalkyl),
         —(CH₂)ₘ-(4- to 6-membered heterocycloalkyl)-,
         —(CH₂)ₘ-(4- to 6-membered heterocycloalkyl)-C(O)—, or
         —(CH₂)ₘ-(4- to 6-membered heterocycloalkyl)-O—,
L₃ is a bond, —C₁₋₈ alkylenyl, 4- to 8-membered heterocycloalkyl, —C₃-C₁₀ cycloalkyl, or (4- to 8-membered heterocycloalkyl)-(CH₂)ₘ—;
X₁ is CH or N;
X₂ is CH or N;
X₇ is O, S, NH, or a bond;
m is independently at each occurrence an integer from 0 to 16;
n is an integer from 0 to 2; and
p is an integer from 0 to 3.

### Embodiment 20

The compound according to any one of the preceding embodiments, wherein the compound is selected from:
6-{[1-(3-{4-[2-(2,6-dioxopiperidin-3-yl)-1-oxo-2,3-dihydro-1H-isoindol-5-yl]piperazin-1-yl}propanoyl)piperidin-4-yl]methoxy}-4-{6-[(1R,5S)-6-[(6-methoxypyridin-3-yl)methyl]-3,6-diazabicyclo[3.1.1]heptan-3-yl]pyridin-3-yl}pyrazolo[1,5-a]pyridine-3-carbonitrile;
6-{[1-(7-{4-[2-(2,6-dioxopiperidin-3-yl)-1-oxo-2,3-dihydro-1H-isoindol-5-yl]piperazin-1-yl}heptanoyl)piperidin-4-yl]methoxy}-4-{6-[(1R,5S)-6-[(6-methoxypyridin-3-yl)methyl]-3,6-diazabicyclo[3.1.1]heptan-3-yl]pyridin-3-yl}pyrazolo[1,5-a]pyridine-3-carbonitrile;
6-{[1-(3-{4-[2-(2,6-dioxopiperidin-3-yl)-1-oxo-2,3-dihydro-1H-isoindol-5-yl]piperidin-1-yl}propanoyl)piperidin-4-yl]methoxy}-4-{6-[(1R,5S)-6-[(6-methoxypyridin-3-yl)methyl]-3,6-diazabicyclo[3.1.1]heptan-3-yl]pyridin-3-yl}pyrazolo[1,5-a]pyridine-3-carbonitrile;
6-{[1-(5-{4-[2-(2,6-dioxopiperidin-3-yl)-1-oxo-2,3-dihydro-1H-isoindol-5-yl]piperidin-1-yl}pentanoyl)piperidin-4-yl]methoxy}-4-{6-[(1R,5S)-6-[(6-methoxypyridin-3-yl)methyl]-3,6-diazabicyclo[3.1.1]heptan-3-yl]pyridin-3-yl}pyrazolo[1,5-a]pyridine-3-carbonitrile;
6-{[1-(3-{4-[2-(2,6-dioxopiperidin-3-yl)-3-oxo-2,3-dihydro-1H-isoindol-5-yl]piperazin-1-yl}propanoyl)piperidin-4-yl]methoxy}-4-{6-[(1R,5S)-6-[(6-methoxypyridin-3-yl)methyl]-3,6-diazabicyclo[3.1.1]heptan-3-yl]pyridin-3-yl}pyrazolo[1,5-a]pyridine-3-carbonitrile;
6-{[1-(7-{4-[2-(2,6-dioxopiperidin-3-yl)-3-oxo-2,3-dihydro-1H-isoindol-5-yl]piperazin-1-yl}heptanoyl)piperidin-4-yl]methoxy}-4-{6-[(1R,5S)-6-[(6-methoxypyridin-3-yl)methyl]-3,6-diazabicyclo[3.1.1]heptan-3-yl]pyridin-3-yl}pyrazolo[1,5-a]pyridine-3-carbonitrile;
6-{[1-(3-{4-[2-(2,6-dioxopiperidin-3-yl)-3-oxo-2,3-dihydro-1H-isoindol-5-yl]piperidin-1-yl}propanoyl)piperidin-4-yl]methoxy}-4-{6-[(1R,5S)-6-[(6-methoxypyridin-3-yl)methyl]-3,6-diazabicyclo[3.1.1]heptan-3-yl]pyridin-3-yl}pyrazolo[1,5-a]pyridine-3-carbonitrile; or
6-{[1-(7-{4-[2-(2,6-dioxopiperidin-3-yl)-3-oxo-2,3-dihydro-1H-isoindol-5-yl]piperidin-1-yl}heptanoyl)piperidin-4-yl]methoxy}-4-{6-[(1R,5S)-6-[(6-methoxypyridin-3-yl)methyl]-3,6-diazabicyclo[3.1.1]heptan-3-yl]pyridin-3-yl}pyrazolo[1,5-a]pyridine-3-carbonitrile.

### Embodiment 21

A pharmaceutical composition comprising a compound of any one of the preceding embodiments or a pharmaceutically acceptable salt thereof, and a pharmaceutically acceptable excipient.

### Embodiment 22

A method of treating a RET mediated disorder comprising administering an effective amount of a compound of any one of the preceding embodiments or a pharmaceutically acceptable salt thereof, optionally in a pharmaceutical composition, to a patient in need thereof.

### Embodiment 23

The method according to any one of the preceding embodiments, wherein the patient is a human.

### Embodiment 24

The method according to any one of the preceding embodiments, wherein the RET mediated disorder is cancer.

### Embodiment 25

The method according to any one of the preceding embodiments, wherein the cancer is non-small cell lung cancer.

### Embodiment 26

The method according to any one of the preceding embodiments, wherein the cancer has metastasized to the brain.

### Embodiment 27

The method according to any one of the preceding embodiments, wherein the RET mediated disorder is mediated by a mutant RET.

### Embodiment 28

The method according to any one of the preceding embodiments, wherein the RET mediated disorder is a relapsed or refractory cancer.

### Embodiment 29

The compound according to any one of the preceding embodiments, or a pharmaceutically acceptable salt thereof, optionally in a pharmaceutical composition, for use in the treatment of a RET mediated disorder.

### Embodiment 30

The compound according to any one of the preceding embodiments, wherein the RET mediated disorder is cancer.

### Embodiment 31

The compound according to any one of the preceding embodiments, wherein the cancer is non-small cell lung cancer.

### Embodiment 32

The compound according to any one of the preceding embodiments, wherein the cancer has metastasized to the brain.

### Embodiment 33

The compound according to any one of the preceding embodiments, wherein the RET mediated disorder is mediated by a mutant RET.

### Embodiment 34

The compound according to any one of the preceding embodiments, wherein the RET mediated disorder is a relapsed or refractory cancer.

### Embodiment 35

The use of a compound according to any one of the preceding embodiments, or a pharmaceutically acceptable salt thereof, optionally in a pharmaceutical composition, in the treatment of a RET mediated disorder.

### Embodiment 36

The use according to any one of the preceding embodiments, wherein the RET mediated disorder is cancer.

### Embodiment 37

The use according to any one of the preceding embodiments, wherein the cancer is non-small cell lung cancer.

### Embodiment 38

The use according to any one of the preceding embodiments, wherein the cancer has metastasized to the brain.

### Embodiment 39

The use according to any one of the preceding embodiments, wherein the RET mediated disorder is mediated by a mutant RET.

### Embodiment 40

The use according to any one of the preceding embodiments, wherein the RET mediated disorder is a relapsed or refractory cancer.

### Embodiment 41

The use of a compound according to any one of the preceding embodiments, a pharmaceutically acceptable salt thereof, optionally in a pharmaceutical composition, in the manufacture of a medicament for the treatment of a RET mediated disorder.

### Embodiment 42

The use according to any one of the preceding embodiments, wherein the RET mediated disorder is cancer.

### Embodiment 43

The use according to any one of the preceding embodiments wherein the cancer is non-small cell lung cancer.

### Embodiment 44

The use according to any one of the preceding embodiments, wherein the cancer has metastasized to the brain.

### Embodiment 45

The use according to any one of the preceding embodiments, wherein the RET mediated disorder is mediated by a mutant RET.

### Embodiment 46

The use according to any one of the preceding embodiments, wherein the RET mediated disorder is a relapsed or refractory cancer.

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

What is claimed is:

1. A compound of Formula I: 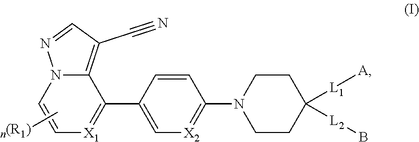
or a pharmaceutically acceptable salt, solvate, isomer, enantiomer, or tautomer thereof,
wherein
R₁ is —OCH₃, —OCH₂CH₃, —OCF₃, —OCH₂CH₂OH, —OCH₂CH₂F, halogen, or 1-methyl-pyrazol-4-yl;
A is independently H, OH, C₁-C₆ alkyl, C₃-C₈ cycloalkyl, aryl, or heteroaryl, wherein the alkyl, cycloalkyl, aryl or heteroaryl is optionally substituted with one or more R₂, wherein R₂ is independently halogen, OH, C₁-C₃ alkyl, or C₁-C₃ alkoxy;
B is a moiety that is capable of binding a E3 ligase;
L₁ is independently a bond, —C₁-C₃ alkylenyl-, —NHCO—, —CONH—, —O—, or —NH—;
L₂ is independently —(CH₂)ₚ-(4- to 12-heterocycloalkyl)-Y₁—C(O)—Y₂—, —(CH₂)ₚ-(4- to 12-heterocycloalkyl)-C(O)—Y₁-Y₂—, —(CH₂)ₚ-(4- to 12-heterocycloalkyl)-C(O)—Y₁—C(O)—Y₂—, —(CH₂)ₚ-(4- to 12-heterocycloalkyl)-Y₁-Y₂—, —(OCH₂CH₂)ₚ-(4- to 12-heterocycloalkyl)-Y₂—, —(CH₂OCH₂C(O))ₚ-(4- to 12-heterocycloalkyl)-Y₂—, —(CH₂OCH₂C(O)NH)ₚ—Y₂—, —(CH₂)ₚ-(4- to 12-heterocycloalkyl)-Y₁—(CH₂CH₂O)ₚ—C(O)—Y₂—, —(CH₂)ₚ-(4- to 12-heterocycloalkyl)-Y₁—(CH₂CH₂O)ₚ—Y₂—, —(CH₂)ₚ-(4- to 12-heterocycloalkyl)-Y₁—(OCH₂CH₂)ₚ—Y₂—, —(CH₂)ₚ-(4- to 12-heterocycloalkyl)-Y₁—(OCH₂CH₂)ₚ—C(O)—(OCH₂CH₂)ₚ—Y₂, —(CH₂OCH₂)ₚ-(heteroaryl)-Y₂—, —NHC(O)-(4- to 12-heterocycloalkyl)-Y₁—C(O)—Y₂—, —NHC(O)—(CH₂)ₚ-(4- to 12-heterocycloalkyl)-Y₁—C(O)—Y₂—, —NHC(O)—(CH₂)ₚ-(4- to 12-heterocycloalkyl)-Y₂—, —C(O)NH—Y₁-Y₂—;
—(CH₂)ₚ—NH—Y₁-Y₂—;
—C(O)NH—(CH₂CH₂O)ₚ—Y₂—,
—C(O)—(CH₂CH₂O)ₚ—Y₂—,
—(CH₂OCH₂)ₚ—NR₃C(O)—(CH₂CH₂O)ₚ—Y₂—,
—(CH₂)ₚ—NR₃C(O)—(CH₂CH₂O)ₚ—Y₂—,
—(CH₂OCH₂CH₂)ₚ-(4- to 12-heterocycloalkyl)-Y₁—C(O)—(CH₂CH₂O)ₚ—Y₂—, or —(CH₂OCH₂)ₚ—C(O)NH—(CH₂CH₂O)ₚ—Y₂—,
wherein
R₃ is —H or —C₁-C₃ alkyl,
Y₁ is independently a bond, —C₁₋₈ alkylenyl, 4- to 12-membered heterocycloalkyl, or —C₃-C₁₀ cycloalkyl;
Y₂ is independently
a bond,
—C₁-C₃ alkyl-,
—(CH₂)ₘ—O—,
—O—(CH₂)ₘ-(4- to 6-membered heterocycloalkyl),
—(CH₂)ₘ—O—(CH₂)ₘ—NH—,
—(CH₂)ₘ—NH—,
—(CH₂)ₘ—NHC(O)—(CH₂)ₘO—,
—(CH₂)ₘ—C(O)NH—(CH₂)ₘ—,
—(CH₂)ₘ—C₃-C₁₀ cycloalkyl-,
—(CH₂)ₘ-aryl,
—(CH₂)ₘ-heteroaryl-,
—(CH₂)ₘ-(4- to 6-membered heterocycloalkyl)-,
—(CH₂)ₘ-(4- to 6-membered heterocycloalkyl)-C(O)—,
—(CH₂)ₘ-(4- to 6-membered heterocycloalkyl)-C(O)—(CH₂)ₘ—,
—(CH₂)ₘ-(4- to 6-membered heterocycloalkyl)-C(O)—(CH₂)ₘ—O—,
—(CH₂)ₘ—C₃-C₁₀ cycloalkyl-C(O)NH—, or
—(CH₂)ₘ-(4- to 6-membered heterocycloalkyl)-O—,
X₁ is CH or N;
X₂ is CH or N;
m is independently at each occurrence an integer from 0 to 16;
n is an integer from 0 to 2; and
p is an integer from 0 to 3.

2. The compound of claim 1, wherein the compound has Formula I(a): 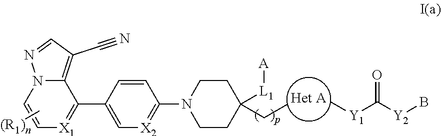
wherein Het A is 4- to 6-membered heterocycloalkyl.

3. The compound of claim 1, wherein the compound has Formula I(b): 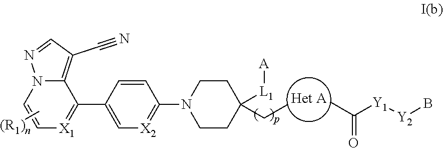
wherein Het A is 4- to 6-membered heterocycloalkyl.

4. The compound of claim 1, wherein the compound has Formula I(c): 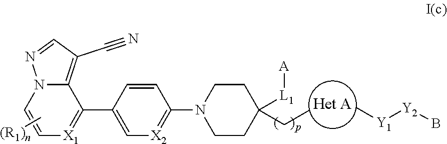
wherein Het A is 4- to 6-membered heterocycloalkoxy.

5. The compound of claim 1, wherein the compound has Formula I(d): 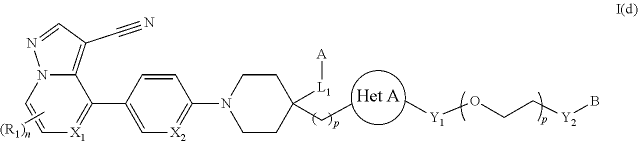
wherein Het A is 4- to 6-membered heterocycloalkyl.

6. The compound of claim 1, wherein the compound has Formula I(e): 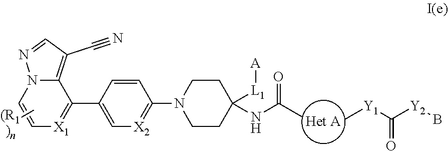
wherein Het A is 4- to 6-membered heterocycloalkyl.

7. The compound of claim 1, wherein the compound has Formula I(f): 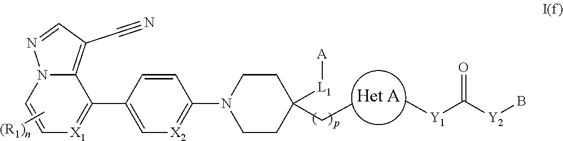
wherein Het A is 4- to 6-membered heterocycloalkyl.

8. The compound of claim 1, wherein the compound has Formula I(g):

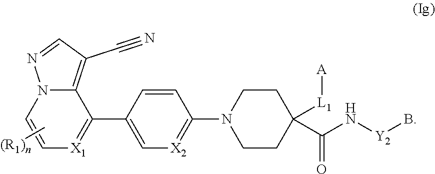

9. The compound of claim 1, wherein the compound has Formula I(h):

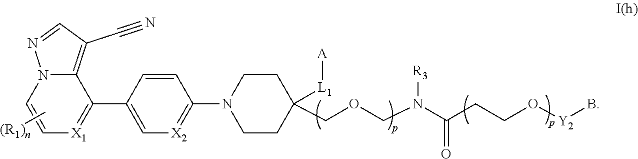

10. The compound of claim 1, wherein moiety B capable of binding a E3 ligase is of the structure:

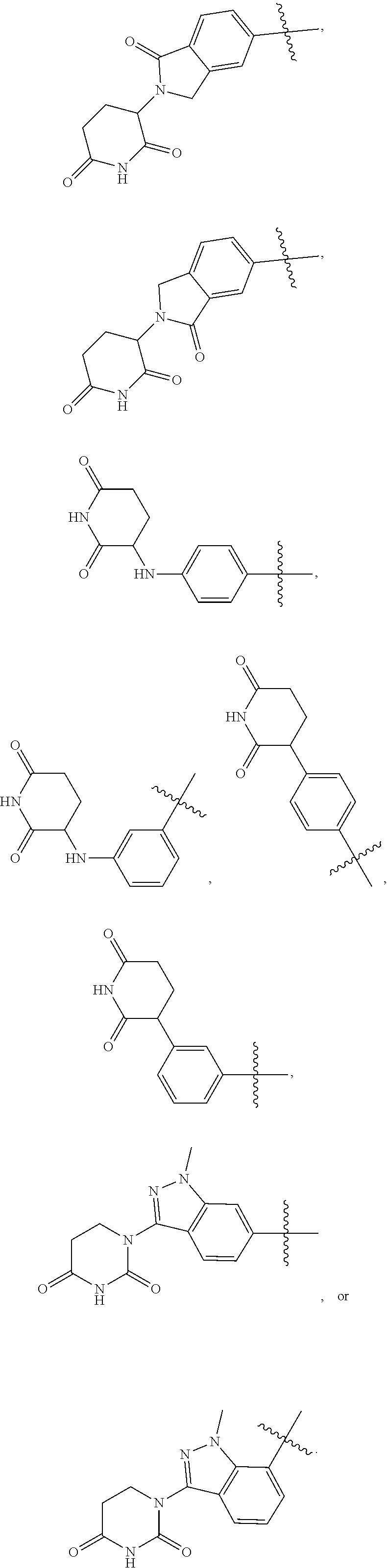

11. The compound of claim 1, wherein moiety B capable of binding a E3 ligase is of the structure:

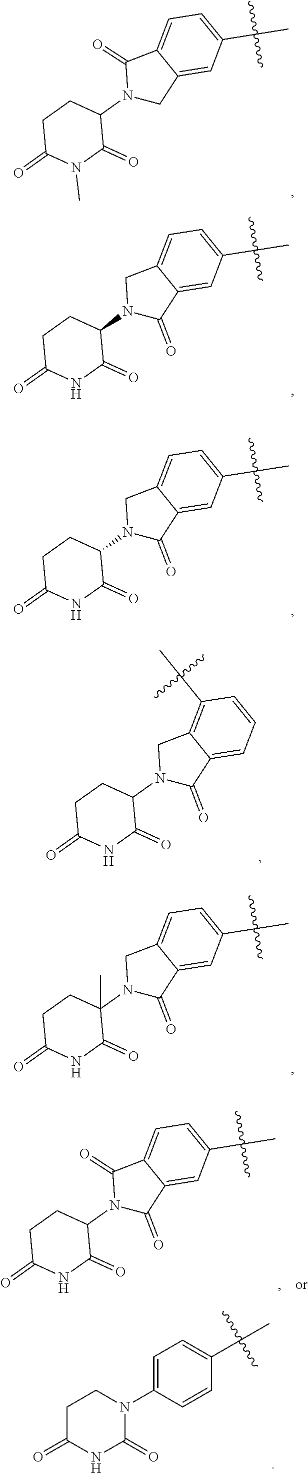

12. The compound of claim 1, wherein A is of the structure:

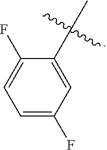 [SMILES: *c1cc(F)ccc1F]

13. The compound of claim 1, wherein the compound is selected from:
N-(1-(5-(3-cyano-6-ethoxypyrazolo[1,5-a]pyridin-4-yl)pyridin-2-yl)-4-((4-(7-(4-(2-(2,6-dioxopiperidin-3-yl)-1-oxoisoindolin-5-yl)piperazin-1-yl)heptanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(7-(4-(2-(2,6-dioxopiperidin-3-yl)-1-oxoisoindolin-5-yl)piperazin-1-yl)heptanoyl)piperazin-1-yl)methyl)piperidin-4-yl)-3-fluoropicolinamide;
N-(1-(5-(3-cyano-6-ethoxypyrazolo[1,5-a]pyridin-4-yl)pyridin-2-yl)-4-((4-(7-(4-(2-(2,6-dioxopiperidin-3-yl)-1-oxoisoindolin-5-yl)piperazin-1-yl)heptanoyl)piperazin-1-yl)methyl)piperidin-4-yl)-3-methylbutanamide;
N-(1-(5-(3-cyano-6-ethoxypyrazolo[1,5-a]pyridin-4-yl)pyridin-2-yl)-4-((4-(3-(4-(2-(2,6 dioxopiperidin-3-yl)-3-oxoisoindolin-5-yl)piperidin-1-yl)propanoyl)piperazin-1-yl)methyl)piperidin-4-yl)-3-fluoropicolinamide;
N-(1-(5-(3-cyano-6-ethoxypyrazolo[1,5-a]pyridin-4-yl)pyridin-2-yl)-4-((4-(5-(4-(2-(2,6-dioxopiperidin-3-yl)-1-oxoisoindolin-5-yl)piperazin-1-yl)pentanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(2-(4-(2-(2,6-dioxopiperidin-3-yl)-1-oxoisoindolin-5-yl)piperazin-1-yl)acetyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(7-(4-(2-(2,6-dioxopiperidin-3-yl)-1-oxoisoindolin-5-yl)piperazin-1-yl)heptanoyl)piperazin-1-yl)methyl)piperidin-4-yl)cyclopropanecarboxamide;
N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)propanoyl)piperazin-1-yl)methyl)piperidin-4-yl)-3-methylbutanamide;
N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)propanoyl)piperazin-1-yl)methyl)piperidin-4-yl)cyclopropanecarboxamide;
N-(1-(5-(3-cyano-6-ethoxypyrazolo[1,5-a]pyridin-4-yl)pyridin-2-yl)-4-((4-(5-(4-(2-(2,6-dioxopiperidin-3-yl)-1-oxoisoindolin-5-yl)piperidin-1-yl)pentanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(7-(4-(2-(2,6-dioxopiperidin-3-yl)-1-oxoisoindolin-5-yl)piperidin-1-yl)heptanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperazin-1-yl)propanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(5-(4-(2-(2,6-dioxopiperidin-3-yl)-3-oxoisoindolin-5-yl)piperazin-1-yl)pentanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(4-(4-(2-(2,6-dioxopiperidin-3-yl)-1-oxoisoindolin-5-yl)piperazin-1-yl)butanoyl)piperazin-1-yl)methyl)piperidin-4-yl)-2,5-difluorobenzamide;
N-[1-(5-{3-cyano-6-ethoxypyrazolo[1,5-a]pyridin-4-yl}pyridin-2-yl)-4-{[4-(3-{4-[2-(2,6-dioxopiperidin-3-yl)-1-oxo-2,3-dihydro-1H-isoindol-5-yl]piperidin-1-yl}propanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(7-{4-[2-(2,6-dioxopiperidin-3-yl)-3-oxo-2,3-dihydro-1H-isoindol-5-yl]piperazin-1-yl}heptanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(3-{4-[2-(2,6-dioxopiperidin-3-yl)-3-oxo-2,3-dihydro-1H-isoindol-5-yl]piperidin-1-yl}propanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(5-{4-[2-(2,6-dioxopiperidin-3-yl)-3-oxo-2,3-dihydro-1H-isoindol-5-yl]piperidin-1-yl}pentanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(7-{4-[2-(2,6-dioxopiperidin-3-yl)-3-oxo-2,3-dihydro-1H-isoindol-5-yl]piperidin-1-yl}heptanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(7-{[4-(2,6-dioxopiperidin-3-yl)phenyl]amino}heptanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(9-{[4-(2,6-dioxopiperidin-3-yl)phenyl]amino}nonanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-({4-[7-({3-[(2,6-dioxopiperidin-3-yl)amino]phenyl}amino)heptanoyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[9-({3-[(2,6-dioxopiperidin-3-yl)amino]phenyl}amino) nonanoyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-{[4-(9-{[3-(2,4-dioxo-1,3-diazinan-1-yl)-1-methyl-1H-indazol-6-yl]amino}nonanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(9-{[3-(2,6-dioxopiperidin-3-yl)-1-methyl-1H-indazol-6-yl]amino}nonanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-({4-[5-(2-{[2-(2,6-dioxopiperidin-3-yl)-1-oxo-2,3-dihydro-1H-isoindol-4-yl]oxy}acetamido)pentanoyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[7-(2-{[2-(2,6-dioxopiperidin-3-yl)-1-oxo-2,3-dihydro-1H-isoindol-4-yl]oxy}acetamido)heptanoyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[11-(2-{[2-(2,6-dioxopiperidin-3-yl)-1-oxo-2,3-dihydro-1H-isoindol-4-yl]oxy}acetamido)undecanoyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[13-(2-{[2-(2,6-dioxopiperidin-3-yl)-1-oxo-2,3-dihydro-1H-isoindol-4-yl]oxy}acetamido)tridecanoyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[15-(2-{[2-(2,6-dioxopiperidin-3-yl)-1-oxo-2,3-dihydro-1H-isoindol-4-yl]oxy}acetamido)pentadecanoyl]piperazin-1-yl}methyl)piperidin-4-yl]-2,5-difluorobenzamide;
N-[1-[5-(3-cyano-6-ethoxy-pyrazolo[1,5-a]pyridin-4-yl)-2-pyridyl]-4-[[2-[4-[4-[2-(2,6-dioxo-3-piperidyl)-1-oxo-isoindolin-5-yl]piperazin-1-yl]butylamino]-2-oxo-ethoxy]methyl]-4-piperidyl]-2,5-difluoro-benzamide;
N-[1-[5-(3-cyano-6-ethoxy-pyrazolo[1,5-a]pyridin-4-yl)-2-pyridyl]-4-[2-[4-[3-[4-[2-(2,6-dioxo-3-piperidyl)-3-oxo-isoindolin-5-yl]-1-piperidyl]propanoyl]piperazin-1-yl]ethoxymethyl]-4-piperidyl]-2,5-difluoro-benzamide;
N-[1-[5-(3-cyano-6-ethoxy-pyrazolo[1,5-a]pyridin-4-yl)-2-pyridyl]-4-[2-[4-[3-[4-[2-(2,6-dioxo-3-piperidyl)-3-oxo-isoindolin-5-yl]piperazin-1-yl]propanoyl]piperazin-1-yl]ethoxymethyl]-4-piperidyl]-2,5-difluoro-benzamide;
N-[1-[5-(3-cyano-6-ethoxy-pyrazolo[1,5-a]pyridin-4-yl)-2-pyridyl]-4-[2-[4-[3-[4-[2-(2,6-dioxo-3-piperidyl)-1-oxo-isoindolin-5-yl]piperazin-1-yl]propanoyl]piperazin-1-yl]ethoxymethyl]-4-piperidyl]-2,5-difluoro-benzamide;
N-[1-[5-(3-cyano-6-ethoxy-pyrazolo[1,5-a]pyridin-4-yl)-2-pyridyl]-4-[2-[4-[5-[4-[2-(2,6-dioxo-3-piperidyl)-1-oxo-isoindolin-5-yl]piperazin-1-yl]pentanoyl]piperazin-1-yl]ethoxymethyl]-4-piperidyl]-2,5-difluoro-benzamide;
N-[1-[5-(3-cyano-6-ethoxy-pyrazolo[1,5-a]pyridin-4-yl)-2-pyridyl]-4-[[2-[4-[[4-[2-(2,6-dioxo-3-piperidyl)-1-oxo-isoindolin-5-yl]piperazin-1-yl]methyl]-1-piperidyl]-2-oxo-ethoxy]methyl]-4-piperidyl]-2,5-difluoro-benzamide;
N-[1-[5-(3-cyano-6-ethoxy-pyrazolo[1,5-a]pyridin-4-yl)-2-pyridyl]-4-[2-[4-[3-[4-[2-(2,6-dioxo-3-piperidyl)-3-oxo-isoindolin-5-yl]-1-piperidyl]propyl]piperazin-1-yl]ethoxymethyl]-4-piperidyl]-2,5-difluoro-benzamide;
N-[1-[5-(3-cyano-6-ethoxy-pyrazolo[1,5-a]pyridin-4-yl)-2-pyridyl]-4-[2-[6-[4-[2-(2,6-dioxo-3-piperidyl)-1-oxo-isoindolin-5-yl]piperazin-1-yl]hexylamino]-2-oxo-ethoxy]methyl]-4-piperidyl]-2,5-difluoro-benzamide;
N-(1-(5-(3-cyano-6-ethoxypyrazolo[1,5-a]pyridin-4-yl)pyridin-2-yl)-4-((4-(3-(4-(3-(2,4-dioxotetrahydropyrimidin-1(2H)-yl)-1-methyl-1H-indazol-6-yl)piperazin-1-yl)propanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(5-(4-(3-(2,4-dioxotetrahydropyrimidin-1(2H)-yl)-1-methyl-1H-indazol-6-yl)piperazin-1-yl)pentanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(7-(4-(3-(2,4-dioxotetrahydropyrimidin-1(2H)-yl)-1-methyl-1H-indazol-6-yl)piperazin-1-yl)heptanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(6-(4-(3-(2,4-dioxotetrahydropyrimidin-1(2H)-yl)-1-methyl-1H-indazol-6-yl)piperidin-1-yl)hexanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(4-(4-(3-(2,4-dioxotetrahydropyrimidin-1(2H)-yl)-1-methyl-1H-indazol-6-yl)piperazin-1-yl)butanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(2-(4-(3-(2,4-dioxotetrahydropyrimidin-1(2H)-yl)-1-methyl-1H-indazol-6-yl)piperidin-1-yl)acetyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(3-(4-(3-(2,4-dioxotetrahydropyrimidin-1(2H)-yl)-1-methyl-1H-indazol-6-yl)piperidin-1-yl)propanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(4-(4-(3-(2,4-dioxotetrahydropyrimidin-1(2H)-yl)-1-methyl-1H-indazol-6-yl)piperidin-1-yl)butanoyl)piperazin-1-yl)methyl)piperidin-4-yl)-2,5-difluorobenzamide;
N-[1-[5-(3-cyano-6-ethoxy-pyrazolo[1,5-a]pyridin-4-yl)-2-pyridyl]-4-[[1-[6-[4-[2-(2,6-dioxo-3-piperidyl)-1-oxo-isoindolin-5-yl]piperazin-1-yl]hexyl]triazol-4-yl]methoxymethyl]-4-piperidyl]-2,5-difluoro-benzamide;
N-[1-[5-(3-cyano-6-ethoxy-pyrazolo[1,5-a]pyridin-4-yl)-2-pyridyl]-4-[[1-[4-[4-[2-(2,6-dioxo-3-piperidyl)-1-oxo-isoindolin-5-yl]piperazin-1-yl]butyl]triazol-4-yl]methoxymethyl]-4-piperidyl]-2,5-difluoro-benzamide;
N-[1-[5-(3-cyano-6-ethoxy-pyrazolo[1,5-a]pyridin-4-yl)-2-pyridyl]-4-[[1-[2-[4-[2-(2,6-dioxo-3-piperidyl)-1-oxo-isoindolin-5-yl]piperazin-1-yl]ethyl]triazol-4-yl]methoxymethyl]-4-piperidyl]-2,5-difluoro-benzamide;
N-(1-(5-(3-cyano-6-ethoxypyrazolo[1,5-a]pyridin-4-yl)pyridin-2-yl)-4-(((1-(3-(4-(2-(2,6-dioxopiperidin-3-yl)-1-oxoisoindolin-5-yl)piperazin-1-yl) propyl)-1H-1,2,3-triazol-4-yl) methoxy)methyl)piperidin-4-yl)-2,5-difluorobenzamide;
N-[4-benzyl-1-[5-(3-cyano-6-ethoxy-pyrazolo[1,5-a]pyridin-4-yl)-2-pyridyl]-4-piperidyl]-4-[3-[4-[2-(2,6-dioxo-3-piperidyl)-3-oxo-isoindolin-5-yl]-1-piperidyl]propanoyl]piperazine-1-carboxamide;
N-[4-benzyl-1-[5-(3-cyano-6-ethoxy-pyrazolo[1,5-a]pyridin-4-yl)-2-pyridyl]-4-piperidyl]-1-[3-[4-[2-(2,6-dioxo-3-piperidyl)-3-oxo-isoindolin-5-yl]-1-piperidyl]propanoyl]piperidine-4-carboxamide;
N-[4-benzyl-1-[5-(3-cyano-6-ethoxy-pyrazolo[1,5-a]pyridin-4-yl)-2-pyridyl]-4-piperidyl]-2-[4-[3-[4-[2-(2,6-dioxo-3-piperidyl)-3-oxo-isoindolin-5-yl]-1-piperidyl]propanoyl]piperazin-1-yl]acetamide;
N-[4-benzyl-1-[5-(3-cyano-6-ethoxy-pyrazolo[1,5-a]pyridin-4-yl)-2-pyridyl]-4-piperidyl]-2-[1-[3-[4-[2-(2,6-dioxo-3-piperidyl)-3-oxo-isoindolin-5-yl]-1-piperidyl]propanoyl]-4-piperidyl]acetamide;
N-[4-benzyl-1-[5-(3-cyano-6-ethoxy-pyrazolo[1,5-apyridin-4-yl)-2-pyridyl]-4-piperidyl]-4-[[4-[2-(2,6-dioxo-3-piperidyl)-3-oxo-isoindolin-5-yl]-1-piperidyl]methyl]piperidine-1-carboxamide;
N-[4-benzyl-1-[5-(3-cyano-6-ethoxy-pyrazolo[1,5-a]pyridin-4-yl)-2-pyridyl]-4-piperidyl]-4-[4-[2-(2,6-dioxo-3-piperidyl)-3-oxo-isoindolin-5-yl]-1-piperidyl]piperidine-1-carboxamide;
N-[4-benzyl-1-[5-(3-cyano-6-ethoxy-pyrazolo[1,5-a]pyridin-4-yl)-2-pyridyl]-4-piperidyl]-2-[4-[[4-[2-(2,6-dioxo-3-piperidyl)-3-oxo-isoindolin-5-yl]-1-piperidyl]methyl]-1-piperidyl]acetamide;
N-(1-(5-(3-cyano-6-ethoxypyrazolo[1,5-a]pyridin-4-yl)pyridin-2-yl)-4-((4-(3-(4-(4-((2,6-dioxopiperidin-3-yl)amino)phenyl)piperazin-1-yl)propanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(3-(4-(3-((2,6-dioxopiperidin-3-yl)amino)phenyl)piperazin-1-yl)propanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(3-(4-(4-(2,6-dioxopiperidin-3-yl)phenyl)piperazin-1-yl)propanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(3-(4-(3-(2,6-dioxopiperidin-3-yl)phenyl)piperazin-1-yl)propanoyl)piperazin-1-yl)methyl)piperidin-4-yl)-2,5-difluorobenzamide;
N-[1-(5-{3-cyano-6-ethoxypyrazolo[1,5-a]pyridin-4-yl}pyridin-2-yl)-4-{[4-(1-{2-[2-(2,6-dioxopiperidin-3-yl)-1-oxo-2,3-dihydro-1H-isoindol-5-yl]acetyl}azetidin-3-yl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-[(4-{1-[2-(2,6-dioxopiperidin-3-yl)-1-oxo-2,3-dihydro-1H-isoindole-5-carbonyl]azetidin-3-yl}piperazin-1-yl)methyl]piperidin-4-yl]-2,5-difluorobenzamide;
N-[1-(5-{3-cyano-6-ethoxypyrazolo[1,5-a]pyridin-4-yl}pyridin-2-yl)-4-[(4-{1-[3-(2,4-dioxo-1,3-diazinan-1-yl)-1-methyl-1H-indazole-6-carbonyl]azetidin-3-yl}piperazin-1-yl)methyl]piperidin-4-yl]-2,5-difluorobenzamide;
N-[1-(5-{3-cyano-6-ethoxypyrazolo[1,5-a]pyridin-4-yl}pyridin-2-yl)-4-({4-[1-(2-{[2-(2,6-dioxopiperidin-3-yl)-1-oxo-2,3-dihydro-1H-isoindol-5-yl]oxy}acetyl)azetidin-3-yl]piperazin-1-yl}methyl)piperidin-4-yl]-2,5-difluorobenzamide;
N-[4-benzyl-1-(5-{3-cyano-6-ethoxypyrazolo[1,5-a]pyridin-4-yl}pyridin-2-yl)piperidin-4-yl]-1-(3-{4-[2-(2,6-dioxopiperidin-3-yl)-1-oxo-2,3-dihydro-1H-isoindol-5-yl]piperidin-1-yl}propanoyl)piperidine-4-carboxamide;
N-(1-(5-(3-cyano-6-ethoxypyrazolo[1,5-a]pyridin-4-yl)pyridin-2-yl)-4-((4-((4-(2-(2,6-dioxopiperidin-3-yl)-3-oxoisoindolin-5-yl)piperazin-1-yl)methyl)piperidin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(4-(2-(2,6-dioxopiperidin-3-yl)-3-oxoisoindolin-5-yl)piperazin-1-yl)piperidin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-((4-(2-(2,6-dioxopiperidin-3-yl)-1-oxoisoindolin-5-yl)piperazin-1-yl)methyl)piperidin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)azetidine-1-carbonyl)piperidin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)azetidin-1-yl)piperidin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-((3-(4-(2-(2,6-dioxopiperidin-3-yl)-3-oxoisoindolin-5-yl)piperidin-1-yl)azetidin-1-yl)methyl)piperidin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)propanoyl)piperazin-1-yl)methyl)piperidin-4-yl)-1-fluorocyclopropane-1-carboxamide;
N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)cyclobutane-1-carbonyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(1-(1-(2-(2,6-dioxopiperidin-3-yl)-3-oxoisoindolin-5-yl)piperidin-4-yl)azetidin-3-yl)piperazin-1-yl)methyl)piperidin-4-yl)-2,5-difluorobenzamide;
1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)propanoyl)piperazin-1-yl)methyl)-N-isopropylpiperidine-4-carboxamide;
1-(5-(3-Cyano-6-ethoxypyrazolo[1,5-a]pyridin-4-yl)pyridin-2-yl)-4-((4-(3-(4-(2-(2,6-dioxopiperidin-3-yl)-1-oxoisoindolin-5-yl)piperazin-1-yl)propanoyl)piperazin-1-yl)methyl)-N-isopropylpiperidine-4-carboxamide;
1-(5-{3-cyano-6-ethoxypyrazolo[1,5-a]pyridin-4-yl}pyridin-2-yl)-4-{[4-(3-{4-[2-(2,6-dioxopiperidin-3-yl)-3-oxo-2,3-dihydro-1H-isoindol-5-yl]piperidin-1-yl}propanoyl)piperazin-1-yl]methyl}-N-(2-methylpropyl)piperidine-4-carboxamide;
N-(1-(5-(3-cyano-6-ethoxypyrazolo[1,5-a]pyridin-4-yl)pyridin-2-yl)-4-((4-(1-(3-(2,4-dioxotetrahydropyrimidin-1(2H)-yl)-1-methyl-1H-indazol-6-yl)piperidine-4-carbonyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(3-(4-(2-(3-methyl-2,6-dioxopiperidin-3-yl)-3-oxoisoindolin-5-yl)piperidin-1-yl)propanoyl)piperazin-1-yl)methyl)piperidin-4-yl)-2,5-difluorobenzamide;
(S)—N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)propanoyl)piperazin-1-yl)methyl)piperidin-4-yl)-2,5-difluorobenzamide;
(R)—N-(1-(5-(3-cyano-6-ethoxypyrazolo[1,5-a]pyridin-4-yl)pyridin-2-yl)-4-((4-(3-(4-(2-(2,6-dioxopiperidin-3-yl)-3-oxoisoindolin-5-yl)piperidin-1-yl)propanoyl)piperazin-1-yl)methyl)piperidin-4-yl)-2,5-difluorobenzamide;
N-(1-(5-(3-cyano-6-ethoxypyrazolo[1,5-a]pyridin-4-yl)pyridin-2-yl)-4-((4-(6-(4-(2-(2,6-dioxopiperidin-3-yl)-1-oxoisoindolin-5-yl)piperazin-1-yl)spiro[3.3]heptane-2-carbonyl)piperazin-1-yl)methyl)piperidin-4-yl)-2,5-difluorobenzamide;
6-(4-{[1-(5-{3-cyano-6-ethoxypyrazolo[1,5-a]pyridin-4-yl}pyridin-2-yl)-4-(2,5-difluorobenzamido)piperidin-4-yl]methyl}piperazine-1-carbonyl)-N-[3-(2,4-dioxo-1,3-diazinan-1-yl)-1-methyl-1H-indazol-6-yl]spiro[3.3]heptane-2-carboxamide;
N-[1-(5-{3-cyano-6-ethoxypyrazolo[1,5-a]pyridin-4-yl}pyridin-2-yl)-4-{[4-(6-{4-[2-(2,6-dioxopiperidin-3-yl)-1-oxo-2,3-dihydro-1H-isoindol-5-yl]piperazine-1-carbonyl}spiro[3.3]heptane-2-carbonyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(6-{4-[2-(2,6-dioxopiperidin-3-yl)-3-oxo-2,3-dihydro-1H-isoindol-5-yl]piperazine-1-carbonyl}spiro[3.3]heptane-2-carbonyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[9-(3-{4-[2-(2,6-dioxopiperidin-3-yl)-1-oxo-2,3-dihydro-1H-isoindol-5-yl]piperazin-1-yl}propanoyl)-3,9-diazaspiro[5.5]undecan-3-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[9-(3-{4-[2-(2,6-dioxopiperidin-3-yl)-1-oxo-2,3-dihydro-1H-isoindol-5-yl]piperidin-1-yl}propanoyl)-3,9-diazaspiro[5.5]undecan-3-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[6-(3-{4-[2-(2,6-dioxopiperidin-3-yl)-1-oxo-2,3-dihydro-1H-isoindol-5-yl]piperidin-1-yl}propanoyl)-2,6-diazaspiro[3.3]heptan-2-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(3-{9-[2-(2,6-dioxopiperidin-3-yl)-1-oxo-2,3-dihydro-1H-isoindol-5-yl]-3,9-diazaspiro[5.5]undecan-3-yl}-3-oxopropyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(3-{9-[2-(2,6-dioxopiperidin-3-yl)-1-oxo-2,3-dihydro-1H-isoindol-5-yl]-3,9-diazaspiro[5.5]undecan-3-yl}propanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(6-{4-[2-(2,6-dioxopiperidin-3-yl)-1-oxo-2,3-dihydro-1H-isoindol-5-yl]piperidin-1-yl}spiro[3.3]heptane-2-carbonyl)piperazin-1-yl]methyl}piperidin-4-yl]-2,5-difluorobenzamide
N-[1-(5-{3-cyano-6-ethoxypyrazolo[1,5-a]pyridin-4-yl}pyridin-2-yl)-4-{[4-(6-{[3-(2,4-dioxo-1,3-diazinan-1-yl)-1-methyl-1H-indazol-6-yl]amino}spiro[3.3]heptane-2-carbonyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(6-{4-[2-(2,6-dioxopiperidin-3-yl)-1-oxo-2,3-dihydro-1H-isoindol-5-yl]piperazine-1-carbonyl}spiro[3.3]heptan-2-yl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(6-{4-[2-(2,6-dioxopiperidin-3-yl)-1-oxo-2,3-dihydro-1H-isoindol-5-yl]piperidine-1-carbonyl}spiro[3.3]heptan-2-yl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-({9-[2-(2,6-dioxopiperidin-3-yl)-3-oxo-2,3-dihydro-1H-isoindole-5-carbonyl]-3,9-diazaspiro[5.5]undecan-3-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-{[6-(2-{[2-(2,6-dioxopiperidin-3-yl)-1-oxo-2,3-dihydro-1H-isoindol-5-yl]oxy}acetyl)-2,6-diazaspiro[3.3]heptan-2-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-[(9-{2-[2-(2,6-dioxopiperidin-3-yl)-1-oxo-2,3-dihydro-1H-isoindol-5-yl]acetyl}-3,9-diazaspiro[5.5]undecan-3-yl)methyl]piperidin-4-yl]-2,5-difluorobenzamide;
N-[1-(5-{3-cyano-6-ethoxypyrazolo[1,5-a]pyridin-4-yl}pyridin-2-yl)-4-{[9-(2-{[2-(2,6-dioxopiperidin-3-yl)-1-oxo-2,3-dihydro-1H-isoindol-5-yl]oxy}acetyl)-3,9-diazaspiro[5.5]undecan-3-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-({4-[2-(3-{4-[2-(2,6-dioxopiperidin-3-yl)-1-oxo-2,3-dihydro-1H-isoindol-5-yl]piperazin-1-yl}-3-oxopropoxy)ethyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[2-(3-{4-[2-(2,6-dioxopiperidin-3-yl)-3-oxo-2,3-dihydro-1H-isoindol-5-yl]piperidin-1-yl}-3-oxopropoxy)ethyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[2-(2-{[3-(2,4-dioxo-1,3-diazinan-1-yl)-1-methyl-1H-indazol-6-yl]carbamoyl}ethoxy)ethyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[2-(3-{4-[2-(2,6-dioxopiperidin-3-yl)-3-oxo-2,3-dihydro-1H-isoindol-5-yl]piperazin-1-yl}-3-oxopropoxy)ethyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[3-(2-{4-[2-(2,6-dioxopiperidin-3-yl)-1-oxo-2,3-dihydro-1H-isoindol-5-yl]piperazin-1-yl}ethoxy)propanoyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[3-(2-{[3-(2,4-dioxo-1,3-diazinan-1-yl)-1-methyl-1H-indazol-6-yl]amino}ethoxy)propanoyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-[(4-{2-[2-(2,6-dioxopiperidin-3-yl)-3-oxo-2,3-dihydro-1H-isoindol-5-yl]acetyl}piperazin-1-yl)methyl]piperidin-4-yl]-2,5-difluorobenzamide;
N-[1-(5-{3-cyano-6-ethoxypyrazolo[1,5-a]pyridin-4-yl}pyridin-2-yl)-4-{[4-(2-{[2-(2,6-dioxopiperidin-3-yl)-1-oxo-2,3-dihydro-1H-isoindol-5-yl]oxy}acetyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-[(4-{2-[2-(2-{[3-(2,4-dioxo-1,3-diazinan-1-yl)-1-methyl-1H-indazol-6-yl]carbamoyl}ethoxy)ethoxy]ethyl}piperazin-1-yl)methyl]piperidin-4-yl]-2,5-difluorobenzamide;
N-[1-(5-{3-cyano-6-ethoxypyrazolo[1,5-a]pyridin-4-yl}pyridin-2-yl)-4-[(4-{2-[2-(3-{4-[2-(2,6-dioxopiperidin-3-yl)-1-oxo-2,3-dihydro-1H-isoindol-5-yl]piperazin-1-yl}-3-oxopropoxy)ethoxy]ethyl}piperazin-1-yl)methyl]piperidin-4-yl]-2,5-difluorobenzamide;
N-[1-(5-{3-cyano-6-ethoxypyrazolo[1,5-a]pyridin-4-yl}pyridin-2-yl)-4-{[4-({1-[2-(2,6-dioxopiperidin-3-yl)-3-oxo-2,3-dihydro-1H-isoindole-5-carbonyl]piperidin-4-yl}methyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-({4-[(1-{2-[2-(2,6-dioxopiperidin-3-yl)-1-oxo-2,3-dihydro-1H-isoindol-4-yl]acetyl}piperidin-4-yl)methyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[(1-{2-[2-(2,6-dioxopiperidin-3-yl)-1-oxo-2,3-dihydro-1H-isoindol-5-yl]acetyl}piperidin-4-yl)methyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-({4-[(1-{2-[2-(2,6-dioxopiperidin-3-yl)-3-oxo-2,3-dihydro-1H-isoindol-5-yl]acetyl}piperidin-4-yl)methyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-[(4-{[1-(2-{[2-(2,6-dioxopiperidin-3-yl)-1-oxo-2,3-dihydro-1H-isoindol-5-yl]oxy}acetyl)piperidin-4-yl]methyl}piperazin-1-yl)methyl]piperidin-4-yl]-2,5-difluorobenzamide;
N-[1-(5-{3-cyano-6-ethoxypyrazolo[1,5-a]pyridin-4-yl}pyridin-2-yl)-4-{[4-(3-{4-[2-(2,6-dioxopiperidin-3-yl)-3-oxo-2,3-dihydro-1H-isoindol-5-yl]piperazin-1-yl}-3-oxopropyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(3-{4-[2-(2,6-dioxopiperidin-3-yl)-3-oxo-2,3-dihydro-1H-isoindol-5-yl]piperidin-1-yl}-3-oxopropyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[4-(3-{4-[2-(2,6-dioxopiperidin-3-yl)-1,3-dioxo-2,3-dihydro-1H-isoindol-5-yl]piperidin-1-yl}-3-oxopropyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-({4-[3-(4-{[2-(2,6-dioxopiperidin-3-yl)-1-oxo-2,3-dihydro-1H-isoindol-5-yl]oxy}piperidin-1-yl)propanoyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-{[4-(3-{3-[2-(2,6-dioxopiperidin-3-yl)-1-oxo-2,3-dihydro-1H-isoindol-5-yl]azetidin-1-yl}propanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-({4-[3-(3-{[2-(2,6-dioxopiperidin-3-yl)-1-oxo-2,3-dihydro-1H-isoindol-5-yl]oxy}azetidin-1-yl)propanoyl]piperazin-1-yl}methyl)piperidin-4-yl]-2,5-difluorobenzamide;
N-[1-(5-{3-cyano-6-ethoxypyrazolo[1,5-a]pyridin-4-yl}pyridin-2-yl)-4-{[9-(2-{4-[2-(2,6-dioxopiperidin-3-yl)-3-oxo-2,3-dihydro-1H-isoindol-5-yl]piperidin-1-yl}-2-oxoethyl)-3-azaspiro[5.5]undecan-3-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[9-(2-{4-[2-(2,6-dioxopiperidin-3-yl)-1-oxo-2,3-dihydro-1H-isoindol-5-yl]piperidin-1-yl}-2-oxoethyl)-3-azaspiro[5.5]undecan-3-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[9-(2-{4-[2-(2,6-dioxopiperidin-3-yl)-3-oxo-2,3-dihydro-1H-isoindol-5-yl]piperazin-1-yl}-2-oxoethyl)-3-azaspiro[5.5]undecan-3-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-{[9-(2-{4-[2-(2,6-dioxopiperidin-3-yl)-1-oxo-2,3-dihydro-1H-isoindol-5-yl]piperazin-1-yl}-2-oxoethyl)-3-azaspiro[5.5]undecan-3-yl]methyl}piperidin-4-yl]-2,5-difluorobenzamide;
4-[6-(4-{[4-(7-{4-[2-(2,6-dioxopiperidin-3-yl)-1-oxo-2,3-dihydro-1H-isoindol-5-yl]piperazin-1-yl}heptanoyl)piperazin-1-yl]methyl}piperidin-1-yl)pyridin-3-yl]-6-ethoxypyrazolo[1,5-a]pyridine-3-carbonitrile;
4-[6-(4-{[4-(3-{4-[2-(2,6-dioxopiperidin-3-yl)-3-oxo-2,3-dihydro-1H-isoindol-5-yl]piperidin-1-yl}propanoyl)piperazin-1-yl]methyl}piperidin-1-yl)pyridin-3-yl]-6-ethoxypyrazolo[1,5-a]pyridine-3-carbonitrile;
N-[1-(5-{3-cyano-6-ethoxypyrazolo[1,5-a]pyridin-4-yl}pyridin-2-yl)-4-[(methylamino)methyl]piperidin-4-yl]-2,5-difluorobenzamide;
4-[6-(4-{[4-(7-{4-[2-(2,6-dioxopiperidin-3-yl)-1-oxo-2,3-dihydro-1H-isoindol-5-yl]piperazin-1-yl}heptanoyl)piperazin-1-yl]methyl}-4-methylpiperidin-1-yl)pyridin-3-yl]-6-ethoxypyrazolo[1,5-a]pyridine-3-carbonitrile;
4-[6-(4-{[4-(3-{4-[2-(2,6-dioxopiperidin-3-yl)-1-oxo-2,3-dihydro-1H-isoindol-5-yl]piperidin-1-yl}propanoyl)piperazin-1-yl]methyl}-4-methylpiperidin-1-yl)pyridin-3-yl]-6-ethoxypyrazolo[1,5-a]pyridine-3-carbonitrile;
4-[6-(4-{[4-(7-{4-[2-(2,6-dioxopiperidin-3-yl)-1-oxo-2,3-dihydro-1H-isoindol-5-yl]piperazin-1-yl}heptanoyl)piperazin-1-yl]methyl}-4-ethylpiperidin-1-yl)pyridin-3-yl]-6-ethoxypyrazolo[1,5-a]pyridine-3-carbonitrile;
4-[6-(4-{[4-(3-{4-[2-(2,6-dioxopiperidin-3-yl)-1-oxo-2,3-dihydro-1H-isoindol-5-yl]piperidin-1-yl}propanoyl)piperazin-1-yl]methyl}-4-ethylpiperidin-1-yl)pyridin-3-yl]-6-ethoxypyrazolo[1,5-a]pyridine-3-carbonitrile;
4-[6-(4-{[4-(3-{4-[2-(2,6-dioxopiperidin-3-yl)-3-oxo-2,3-dihydro-1H-isoindol-5-yl]piperidin-1-yl}propanoyl)piperazin-1-yl]methyl}-4-ethylpiperidin-1-yl)pyridin-3-yl]-6-ethoxypyrazolo[1,5-a]pyridine-3-carbonitrile;
4-[6-(4-{[4-(2-{[2-(2,6-dioxopiperidin-3-yl)-1-oxo-2,3-dihydro-1H-isoindol-5-yl]oxy}acetyl)piperazin-1-yl]methyl}-4-ethylpiperidin-1-yl)pyridin-3-yl]-6-ethoxypyrazolo[1,5-a]pyridine-3-carbonitrile;
N-(1-(5-(3-cyano-6-ethoxypyrazolo[1,5-a]pyridin-4-yl)pyridin-2-yl)-4-(9-(2-(2,6-dioxopiperidin-3-yl)-1-oxoisoindolin-5-yl)-3,9-diazaspiro[5.5]undecane-3-carbonyl)piperidin-4-yl)-2,5-difluorobenzamide;
N-[1-(5-{3-cyano-6-ethoxypyrazolo[1,5-a]pyridin-4-yl}pyridin-2-yl)-4-{[4-(7-{4-[2-(1-methyl-2,6-dioxopiperidin-3-yl)-1-oxo-2,3-dihydro-1H-isoindol-5-yl]piperazin-1-yl}heptanoyl)piperazin-1-yl]methyl}piperidin-4-yl]-2,5-difluorobenzamide;
N-[1-(5-{3-cyano-6-ethoxypyrazolo[1,5-a]pyridin-4-yl}pyridin-2-yl)-4-[(5-{4-[2-(2,6-dioxopiperidin-3-yl)-1-oxo-2,3-dihydro-1H-isoindol-5-yl]piperazin-1-yl}-N-methylpentanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(7-{4-[2-(2,6-dioxopiperidin-3-yl)-1-oxo-2,3-dihydro-1H-isoindol-5-yl]piperazin-1-yl}-N-methylheptanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(3-{4-[2-(2,6-dioxopiperidin-3-yl)-1-oxo-2,3-dihydro-1H-isoindol-5-yl]piperidin-1-yl}-N-methylpropanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(5-{4-[2-(2,6-dioxopiperidin-3-yl)-1-oxo-2,3-dihydro-1H-isoindol-5-yl]piperidin-1-yl}-N-methylpentanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(7-{4-[2-(2,6-dioxopiperidin-3-yl)-1-oxo-2,3-dihydro-1H-isoindol-5-yl]piperidin-1-yl}-N-methylheptanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(3-{4-[2-(2,6-dioxopiperidin-3-yl)-3-oxo-2,3-dihydro-1H-isoindol-5-yl]piperazin-1-yl}-N-methylpropanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(5-{4-[2-(2,6-dioxopiperidin-3-yl)-3-oxo-2,3-dihydro-1H-isoindol-5-yl]piperazin-1-yl}-N-methylpentanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(7-{4-[2-(2,6-dioxopiperidin-3-yl)-3-oxo-2,3-dihydro-1H-isoindol-5-yl]piperazin-1-yl}-N-methylheptanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(3-{4-[2-(2,6-dioxopiperidin-3-yl)-3-oxo-2,3-dihydro-1H-isoindol-5-yl]piperidin-1-yl}-N-methylpropanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(5-{4-[2-(2,6-dioxopiperidin-3-yl)-3-oxo-2,3-dihydro-1H-isoindol-5-yl]piperidin-1-yl}-N-methylpentanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(7-{4-[2-(2,6-dioxopiperidin-3-yl)-3-oxo-2,3-dihydro-1H-isoindol-5-yl]piperidin-1-yl}-N-methylheptanamido)methyl]piperidin-4-yl]-2,5-difluorobenzamide;
4-[6-(4-{[4-(3-{4-[2-(2,6-dioxopiperidin-3-yl)-3-oxo-2,3-dihydro-1H-isoindol-5-yl]piperidin-1-yl}propanoyl)piperazin-1-yl]methyl}-4-methylpiperidin-1-yl)pyridin-3-yl]-6-ethoxypyrazolo[1,5-a]pyridine-3-carbonitrile;
1-(5-{3-cyano-6-ethoxypyrazolo[1,5-a]pyridin-4-yl}pyridin-2-yl)-4-(2,5-difluorobenzamido)-N-(6-{[4-(2,4-dioxo-1,3-diazinan-1-yl)phenyl]amino}hexyl)piperidine-4-carboxamide;
1-(5-{3-cyano-6-ethoxypyrazolo[1,5-a]pyridin-4-yl}pyridin-2-yl)-4-(2,5-difluorobenzamido)-N-(6-{[3-(2,4-dioxo-1,3-diazinan-1-yl)-1-methyl-1H-indazol-7-yl]amino}hexyl)piperidine-4-carboxamide;
1-(5-{3-cyano-6-ethoxypyrazolo[1,5-a]pyridin-4-yl}pyridin-2-yl)-4-(2,5-difluorobenzamido)-N-(10-{[3-(2,4-dioxo-1,3-diazinan-1-yl)-1-methyl-1H-indazol-7-yl]amino}decyl)piperidine-4-carboxamide;
1-(5-{3-cyano-6-ethoxypyrazolo[1,5-a]pyridin-4-yl}pyridin-2-yl)-4-(2,5-difluorobenzamido)-N-(6-{[3-(2,4-dioxo-1,3-diazinan-1-yl)-1-methyl-1H-indazol-6-yl]amino}hexyl)piperidine-4-carboxamide;
1-(5-{3-cyano-6-ethoxypyrazolo[1,5-a]pyridin-4-yl}pyridin-2-yl)-4-(2,5-difluorobenzamido)-N-(8-{[3-(2,4-dioxo-1,3-diazinan-1-yl)-1-methyl-1H-indazol-6-yl]amino}octyl)piperidine-4-carboxamide;
1-(5-{3-cyano-6-ethoxypyrazolo[1,5-a]pyridin-4-yl}pyridin-2-yl)-4-(2,5-difluorobenzamido)-N-(10-{[3-(2,4-dioxo-1,3-diazinan-1-yl)-1-methyl-1H-indazol-6-yl]amino}decyl)piperidine-4-carboxamide;
1-(5-{3-cyano-6-ethoxypyrazolo[1,5-a]pyridin-4-yl}pyridin-2-yl)-4-(2,5-difluorobenzamido)-N-(4-{4-[2-(2,6-dioxopiperidin-3-yl)-1-oxo-2,3-dihydro-1H-isoindol-5-yl]piperazin-1-yl}butyl)piperidine-4-carboxamide;
1-(5-{3-cyano-6-ethoxypyrazolo[1,5-a]pyridin-4-yl}pyridin-2-yl)-4-(2,5-difluorobenzamido)-N-(6-{4-[2-(2,6-dioxopiperidin-3-yl)-1-oxo-2,3-dihydro-1H-isoindol-5-yl]piperazin-1-yl}hexyl)piperidine-4-carboxamide;
1-(5-{3-cyano-6-ethoxypyrazolo[1,5-a]pyridin-4-yl}pyridin-2-yl)-4-(2,5-difluorobenzamido)-N-(2-{4-[2-(2,6-dioxopiperidin-3-yl)-1-oxo-2,3-dihydro-1H-isoindol-5-yl]piperidin-1-yl}ethyl)piperidine-4-carboxamide;
1-(5-{3-cyano-6-ethoxypyrazolo[1,5-a]pyridin-4-yl}pyridin-2-yl)-4-(2,5-difluorobenzamido)-N-(6-{4-[2-(2,6-dioxopiperidin-3-yl)-1-oxo-2,3-dihydro-1H-isoindol-5-yl]piperidin-1-yl}hexyl)piperidine-4-carboxamide;
1-(5-{3-cyano-6-ethoxypyrazolo[1,5-a]pyridin-4-yl}pyridin-2-yl)-4-(2,5-difluorobenzamido)-N-(2-{4-[2-(2,6-dioxopiperidin-3-yl)-3-oxo-2,3-dihydro-1H-isoindol-5-yl]piperazin-1-yl}ethyl)piperidine-4-carboxamide;
1-(5-{3-cyano-6-ethoxypyrazolo[1,5-a]pyridin-4-yl}pyridin-2-yl)-4-(2,5-difluorobenzamido)-N-(4-{4-[2-(2,6-dioxopiperidin-3-yl)-3-oxo-2,3-dihydro-1H-isoindol-5-yl]piperazin-1-yl}butyl)piperidine-4-carboxamide;
1-(5-{3-cyano-6-ethoxypyrazolo[1,5-a]pyridin-4-yl}pyridin-2-yl)-4-(2,5-difluorobenzamido)-N-(6-{4-[2-(2,6-dioxopiperidin-3-yl)-3-oxo-2,3-dihydro-1H-isoindol-5-yl]piperazin-1-yl}hexyl)piperidine-4-carboxamide;
1-(5-{3-cyano-6-ethoxypyrazolo[1,5-a]pyridin-4-yl}pyridin-2-yl)-4-(2,5-difluorobenzamido)-N-(2-{4-[2-(2,6-dioxopiperidin-3-yl)-3-oxo-2,3-dihydro-1H-isoindol-5-yl]piperidin-1-yl}ethyl)piperidine-4-carboxamide;
1-(5-{3-cyano-6-ethoxypyrazolo[1,5-a]pyridin-4-yl}pyridin-2-yl)-4-(2,5-difluorobenzamido)-N-(4-{4-[2-(2,6-dioxopiperidin-3-yl)-3-oxo-2,3-dihydro-1H-isoindol-5-yl]piperidin-1-yl}butyl)piperidine-4-carboxamide;
1-(5-{3-cyano-6-ethoxypyrazolo[1,5-a]pyridin-4-yl}pyridin-2-yl)-4-(2,5-difluorobenzamido)-N-(6-{4-[2-(2,6-dioxopiperidin-3-yl)-3-oxo-2,3-dihydro-1H-isoindol-5-yl]piperidin-1-yl}hexyl)piperidine-4-carboxamide;
N-[1-(5-{3-cyano-6-ethoxypyrazolo[1,5-a]pyridin-4-yl}pyridin-2-yl)-4-[(5-{4-[2-(2,6-dioxopiperidin-3-yl)-1-oxo-2,3-dihydro-1H-isoindol-5-yl]piperazin-1-yl}-N-(propan-2-yl)pentanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(7-{4-[2-(2,6-dioxopiperidin-3-yl)-1-oxo-2,3-dihydro-1H-isoindol-5-yl]piperazin-1-yl}-N-(propan-2-yl)heptanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(7-{4-[2-(2,6-dioxopiperidin-3-yl)-1-oxo-2,3-dihydro-1H-isoindol-5-yl]piperidin-1-yl}-N-(propan-2-yl)heptanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(3-{4-[2-(2,6-dioxopiperidin-3-yl)-3-oxo-2,3-dihydro-1H-isoindol-5-yl]piperazin-1-yl}-N-(propan-2-yl)propanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(5-{4-[2-(2,6-dioxopiperidin-3-yl)-3-oxo-2,3-dihydro-1H-isoindol-5-yl]piperazin-1-yl}-N-(propan-2-yl)pentanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(7-{4-[2-(2,6-dioxopiperidin-3-yl)-3-oxo-2,3-dihydro-1H-isoindol-5-yl]piperazin-1-yl}-N-(propan-2-yl)heptanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[(5-{4-[2-(2,6-dioxopiperidin-3-yl)-3-oxo-2,3-dihydro-1H-isoindol-5-yl]piperidin-1-yl}-N-(propan-2-yl)pentanamido)methyl]piperidin-4-yl]-2,5-difluorobenzamide;
N-[1-(5-{3-cyano-6-ethoxypyrazolo[1,5-a]pyridin-4-yl}pyridin-2-yl)-4-[9-(2-{4-[2-(2,6-dioxopiperidin-3-yl)-3-oxo-2,3-dihydro-1H-isoindol-5-yl]piperidin-1-yl}-2-oxoethyl)-3-azaspiro[5.5]undecane-3-carbonyl]piperidin-4-yl]-2,5-difluorobenzamide;
N-[1-(5-{3-cyano-6-ethoxypyrazolo[1,5-a]pyridin-4-yl}pyridin-2-yl)-4-[9-(2-{4-[2-(2,6-dioxopiperidin-3-yl)-1-oxo-2,3-dihydro-1H-isoindol-5-yl]piperidin-1-yl}-2-oxoethyl)-3-azaspiro[5.5]undecane-3-carbonyl]piperidin-4-yl]-2,5-difluorobenzamide;
N-[1-(5-{3-cyano-6-ethoxypyrazolo[1,5-a]pyridin-4-yl}pyridin-2-yl)-4-[9-(2-{4-[2-(2,6-dioxopiperidin-3-yl)-3-oxo-2,3-dihydro-1H-isoindol-5-yl]piperazin-1-yl}-2-oxoethyl)-3-azaspiro[5.5]undecane-3-carbonyl]piperidin-4-yl]-2,5-difluorobenzamide;
N-[1-(5-{3-cyano-6-ethoxypyrazolo[1,5-a]pyridin-4-yl}pyridin-2-yl)-4-[9-(2-{4-[2-(2,6-dioxopiperidin-3-yl)-1-oxo-2,3-dihydro-1H-isoindol-5-yl]piperazin-1-yl}-2-oxoethyl)-3-azaspiro[5.5]undecane-3-carbonyl]piperidin-4-yl]-2,5-difluorobenzamide;
(S)—N-(1-(5-(3-cyano-6-ethoxypyrazolo[1,5-a]pyridin-4-yl)pyridin-2-yl)-4-((4-(7-(4-(2-(2,6-dioxopiperidin-3-yl)-3-oxoisoindolin-5-yl)piperazin-1-yl)heptanoyl)piperazin-1-yl)methyl)piperidin-4-yl)-2,5-difluorobenzamide;
(R)—N-(1-(5-(3-cyano-6-ethoxypyrazolo[1,5-a]pyridin-4-yl)pyridin-2-yl)-4-((4-(7-(4-(2-(2,6-dioxopiperidin-3-yl)-3-oxoisoindolin-5-yl)piperazin-1-yl)heptanoyl)piperazin-1-yl)methyl)piperidin-4-yl)-2,5-difluorobenzamide; or
N-[1-(5-{3-cyano-6-ethoxypyrazolo[1,5-a]pyridin-4-yl}pyridin-2-yl)-4-[(7-{4-[2-(2,6-dioxopiperidin-3-yl)-3-oxo-2,3-dihydro-1H-isoindol-5-yl]piperidin-1-yl}-N-(propan-2-yl)heptanamido)methyl]piperidin-4-yl]-2,5-difluorobenzamide.

14. A compound of Formula II: 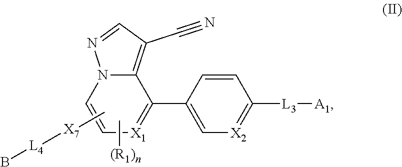
and pharmaceutically acceptable salts, solvates, isomers, enantiomers, and tautomers thereof,
wherein
R₁ is —OCH₂CH₃, —OCH₃, —OCF₃, —OCH₂CH₂OH, —OCH₂CH₂F, 1-methylpyrazol-4-yl, or halogen;
A₁ is independently aryl, or heteroaryl, wherein the aryl or heteroaryl is optionally substituted with one or more R₂; wherein R₂ is independently halogen, OH, C₁-C₃ alkyl, or C₁-C₃ alkoxy;
B is a moiety that is capable of binding a E3 ligase;
L₄ is independently —(CH₂)ₚ-(4- to 12-heterocycloalkyl)-Y₁—C(O)—Y₂—, —(CH₂)ₚ-(4- to 12-heterocycloalkyl)-C(O)—Y₁-Y₂—, —(CH₂)ₚ-(4- to 12-heterocycloalkyl)-Y₁-Y₂—,
wherein
Y₁ is independently —C₁₋₈ alkylenyl, 4- to 12-membered heterocycloalkyl, or —C₃-C₁₀ cycloalkyl;
Y₂ is independently
—C₁-C₃ alkyl-,
—(CH₂)ₘ—O—,
—O—(CH₂)ₘ-(4- to 6-membered heterocycloalkyl),
—(CH₂)ₘ-(4- to 6-membered heterocycloalkyl)-,
—(CH₂)ₘ-(4- to 6-membered heterocycloalkyl)-C(O)—, or
—(CH₂)ₘ-(4- to 6-membered heterocycloalkyl)-O—,
L₃ is a bond, —C₁₋₈ alkylenyl, 4- to 8-membered heterocycloalkyl, —C₃-C₁₀ cycloalkyl, or -(4- to 8-membered heterocycloalkyl)-(CH₂)ₘ—;
X₁ is CH or N;
X₂ is CH or N;
X₇ is O, S, NH, or a bond;
m is independently at each occurrence an integer from 0 to 16;
n is an integer from 0 to 2; and
p is an integer from 0 to 3.

15. The compound of claim 14, wherein the compound is selected from:
6-{[1-(3-{4-[2-(2,6-dioxopiperidin-3-yl)-1-oxo-2,3-dihydro-1H-isoindol-5-yl]piperazin-1-yl}propanoyl)piperidin-4-yl]methoxy}-4-{6-[(1R,5S)-6-[(6-methoxypyridin-3-yl)methyl]-3,6-diazabicyclo[3.1.1]heptan-3-yl]pyridin-3-yl}pyrazolo[1,5-a]pyridine-3-carbonitrile;
6-{[1-(7-{4-[2-(2,6-dioxopiperidin-3-yl)-1-oxo-2,3-dihydro-1H-isoindol-5-yl]piperazin-1-yl}heptanoyl)piperidin-4-yl]methoxy}-4-{6-[(1R,5S)-6-[(6-methoxypyridin-3-yl)methyl]-3,6-diazabicyclo[3.1.1]heptan-3-yl]pyridin-3-yl}pyrazolo[1,5-a]pyridine-3-carbonitrile;
6-{[1-(3-{4-[2-(2,6-dioxopiperidin-3-yl)-1-oxo-2,3-dihydro-1H-isoindol-5-yl]piperidin-1-yl}propanoyl)piperidin-4-yl]methoxy}-4-{6-[(1R,5S)-6-[(6-methoxypyridin-3-yl)methyl]-3,6-diazabicyclo[3.1.1]heptan-3-yl]pyridin-3-yl}pyrazolo[1,5-a]pyridine-3-carbonitrile;
6-{[1-(5-{4-[2-(2,6-dioxopiperidin-3-yl)-1-oxo-2,3-dihydro-1H-isoindol-5-yl]piperidin-1-yl}pentanoyl)piperidin-4-yl]methoxy}-4-{6-[(1R,5S)-6-[(6-methoxypyridin-3-yl)methyl]-3,6-diazabicyclo[3.1.1]heptan-3-yl]pyridin-3-yl}pyrazolo[1,5-a]pyridine-3-carbonitrile;
6-{[1-(3-{4-[2-(2,6-dioxopiperidin-3-yl)-3-oxo-2,3-dihydro-1H-isoindol-5-yl]piperazin-1-yl}propanoyl)piperidin-4-yl]methoxy}-4-{6-[(1R,5S)-6-[(6-methoxypyridin-3-yl)methyl]-3,6-diazabicyclo[3.1.1]heptan-3-yl]pyridin-3-yl}pyrazolo[1,5-a]pyridine-3-carbonitrile;
6-{[1-(7-{4-[2-(2,6-dioxopiperidin-3-yl)-3-oxo-2,3-dihydro-1H-isoindol-5-yl]piperazin-1-yl}heptanoyl)piperidin-4-yl]methoxy}-4-{6-[(1R,5S)-6-[(6-methoxypyridin-3-yl)methyl]-3,6-diazabicyclo[3.1.1]heptan-3-yl]pyridin-3-yl}pyrazolo[1,5-a]pyridine-3-carbonitrile;
6-{[1-(3-{4-[2-(2,6-dioxopiperidin-3-yl)-3-oxo-2,3-dihydro-1H-isoindol-5-yl]piperidin-1-yl}propanoyl)piperidin-4-yl]methoxy}-4-{6-[(1R,5S)-6-[(6-methoxypyridin-3-yl)methyl]-3,6-diazabicyclo[3.1.1]heptan-3-yl]pyridin-3-yl}pyrazolo[1,5-a]pyridine-3-carbonitrile; or
6-{[1-(7-{4-[2-(2,6-dioxopiperidin-3-yl)-3-oxo-2,3-dihydro-1H-isoindol-5-yl]piperidin-1-yl}heptanoyl)piperidin-4-yl]methoxy}-4-{6-[(1R,5S)-6-[(6-methoxypyridin-3-yl)methyl]-3,6-diazabicyclo[3.1.1]heptan-3-yl]pyridin-3-yl}pyrazolo[1,5-a]pyridine-3-carbonitrile.

16. A pharmaceutical composition comprising a compound of claim 1 or a pharmaceutically acceptable salt thereof, and a pharmaceutically acceptable excipient.

17. A method of treating a RET mediated disorder comprising administering an effective amount of a compound of claim 1 or a pharmaceutically acceptable salt thereof, optionally in a pharmaceutical composition, to a patient in need thereof.

18. A compound of claim 1 or a pharmaceutically acceptable salt thereof, optionally in a pharmaceutical composition, for use in the treatment of a RET mediated disorder.

19. Use of a compound of claim 1 or a pharmaceutically acceptable salt thereof, optionally in a pharmaceutical composition, in the treatment of a RET mediated disorder.

20. Use of a compound of claim 1 or a pharmaceutically acceptable salt thereof, optionally in a pharmaceutical composition, in the manufacture of a medicament for the treatment of a RET mediated disorder.
